(19) United States
(12) Patent Application Publication
(10) Pub. No.: US 2003/0040615 A1
(43) Pub. Date: Feb. 27, 2003

(54) Polynucleotides encoding or regulating electron transfer molecules

(76) Inventors: Jeffrey J. Seilhamer, Los Altos Hill, CA (US); Angelo M. Delegeane, Milpitas, CA (US); Susan G. Stuart, Montara, CA (US); Laura L. Stuve, Los Gato, CA (US); Sara J. Mullahy, Mountain View, CA (US); Rebecca E. Naughton, Chester Springs, PA (US)

Correspondence Address: INCYTE GENOMICS, INC., 3160 PORTER DRIVE, PALO ALTO, CA, 94304, US

(21) Appl. No.: 09/535,459

(22) Filed: Mar. 24, 2000

Related U.S. Application Data
(63) Continuation-in-part of application No. 09/008,119, filed on Jan. 16, 1998, now abandoned.
Application No. 09/008,119 is a continuation of application No. 08/100,523, filed on Aug. 3, 1993, now abandoned.
Application No. 08/100,523 is a continuation-in-part of application No. 08/282,991, filed on Jul. 28, 1994, now abandoned.
Application No. 08/282,991 is a continuation-in-part of application No. 08/196,364, filed on Feb. 14, 1994, now abandoned.
Application No. 08/196,364 is a continuation-in-part of application No. 08/100,523, filed on Aug. 3, 1993, now abandoned.
Application No. 08/100,523 is a continuation-in-part of application No. 07/977,780, filed on Nov. 19, 1992.
Application No. 07/977,780 is a continuation-in-part of application No. 07/916,491, filed on Jul. 17, 1992, now abandoned.

(51) Int. Cl. C07H 21/02; C12Q 1/68; C12N 15/00

(52) U.S. Cl. 536/23.5; 435/6; 435/69.1; 435/91.2; 435/252.3; 435/320.1; 536/23.1

(57) ABSTRACT

The present invention provides purified polynucleotides, complements and fragments thereof, identified as encoding or regulating electron transfer molecules. The invention also provides for the use of these polynucleotides in methods to produce polypeptides and in the diagnosis, study, prevention, and treatment of disorders.

This application is a continuation-in-part of the following applications: U.S. application Ser. No. 09/008,119 filed Jan. 16, 1998 which is a continuation-in-part of U.S. application Ser. No. 08/100,523, abandoned, filed Aug. 3, 1993 which is a continuation-in-part of U.S. application Ser. No. 07/977,780 filed Nov. 19, 1992 which is a continuation-in-part of U.S. application Ser. No. 07/916,491, abandoned, filed Jul. 17, 1992; U.S. application Ser. No. 08/438,571 filed May 10, 1995 which is a continuation-in-part of U.S. application Ser. No. 08/282,991, abandoned, filed Jul. 28, 1994 which is a continuation-in-part of U.S. application Ser. No. 08/196,364, abandoned, filed Feb. 14, 1994 which is a continuation-in-part of U.S. application Ser. No. 08/100,523, abandoned, filed Aug. 3, 1993; U.S. application Ser. No. 08/504,732 filed Jul. 20, 1995 which is a continuation-in-part of U.S. application Ser. No. 08/179,873, abandoned, filed Jan. 11, 1994; U.S. application Ser. No. 08/296,757 filed Aug. 26, 1994 which is a continuation-in-part of U.S. application Ser. No. 08/237,491, abandoned, filed Apr. 28, 1994 which is a continuation-in-part of U.S. application Ser. No. 08/197,801, abandoned, filed Feb. 17, 1994 which is a continuation-in-part of U.S. application Ser. No. 08/137,951, abandoned, filed Oct. 14, 1993; U.S. application Ser. No. 08/435,761 filed May 5, 1995 which is a continuation-in-part of U.S. application Ser. No. 08/221,531, abandoned, filed Feb. 2, 1994; U.S. application Ser. No. 08/502, 242 filed Jul. 13, 1995 which is a continuation-in-part of U.S. application Ser. No. 08/413,151, abandoned, filed Mar. 29, 1995 which is a continuation-in-part of U.S. application Ser. No. 08/194,317, abandoned, filed Feb. 4, 1994; U.S. application Ser. No. 08/487,829 filed Jun. 7, 1995 which is a continuation-in-part of U.S. application Ser. No. 08/222,611, abandoned, filed Mar. 31, 1994; U.S. application Ser. No. 08/499,410 filed Jul. 7, 1995 which is a continuation-in-part of U.S. application Ser. No. 08/216,595, abandoned, filed Mar. 22, 1994; U.S. application Ser. No. 08/521,383 filed Aug. 16, 1995 which is a continuation-in-part of U.S. application Ser. No. 08/271,217, abandoned, filed Jun. 27, 1994; U.S. application Ser. No. 08/413,150 filed Mar. 29, 1995 which is a continuation-in-part of U.S. application Ser. No. 08/274,621, abandoned, filed Jul. 8, 1994; U.S. application Ser. No. 08/412,033 filed Mar. 28, 1995 which is a continuation-in-part of U.S. application Ser. No. 08/270,638, abandoned, filed Jul. 1, 1994; U.S. application Ser. No. 08/404,891 filed Mar. 15, 1995 which is a continuation-in-part of U.S. application Ser. No. 08/276,164, abandoned, filed Jul. 15, 1994; U.S. application Ser. No. 08/413,793 filed Mar. 30, 1995 which is a continuation-in-part of U.S. application Ser. No. 08/293,347, abandoned, filed Aug. 19, 1994; U.S. application Ser. No. 08/446,910 filed May 22, 1995 which is a continuation-in-part of U.S. application Ser. No. 08/421,124, abandoned, filed Apr. 12, 1995 which is a continuation-in-part of U.S. application Ser. No. 08/303,241, abandoned, filed Sep. 7, 1994; U.S. application Ser. No. 08/489,200 filed Jun. 9, 1995 which is a continuation-in-part of U.S. application Ser. No. 08/323,523, abandoned, filed Oct. 14, 1994; U.S. application Ser. No. 08/440,743 filed May 12, 1995 which is a continuation-in-part of U.S. application Ser. No. 08/320,011, abandoned, filed Oct. 5, 1994; U.S. application Ser. No. 08/334,881 filed Nov. 4, 1994; U.S. application Ser. No. 08/408,872 filed Mar. 21, 1995 which is a continuation-in-part of U.S. application Ser. No. 08/369,881, abandoned, filed Jan. 5, 1995; U.S. application Ser. No. 08/373,361 filed Jan. 17, 1995; U.S. application Ser. No. 08/972,819 filed Nov. 18, 1997 which is a continuation of U.S. application Ser. No. 08/393,220, abandoned, filed Feb. 23, 1995; U.S. application Ser. No. 08/494,619 filed Jun. 23, 1995; U.S. application Ser. No. 08/964,263 filed Nov. 4, 1997 which is a continuation of U.S. application Ser. No. 08/385,268, abandoned, filed Feb. 7, 1995; U.S. application Ser. No. 08/964,265 filed Nov. 4, 1997 which is a continuation of U.S. application Ser. No. 08/392,180 filed Feb. 22, 1995; U.S. application Ser. No. 08/972,899 filed Nov. 18, 1997 which is a continuation of U.S. application Ser. No. 08/395,244, abandoned, filed Feb. 27, 1995; U.S. application Ser. No. 08/451,242 filed May 25, 1995; U.S. application Ser. No. 08/963,650 filed Nov. 3, 1997 which is a continuation of U.S. application Ser. No. 08/392,715, abandoned, filed Feb. 23, 1995; U.S. application Ser. No. 08/416,401 filed Mar. 31, 1995; U.S. application Ser. No. 08/497,967 filed Jul. 3, 1995 which is a continuation-in-part of U.S. application Ser. No. 08/406,219, abandoned, filed Mar. 16, 1995; U.S. application Ser. No. 08/429,361 filed Apr. 26, 1995; U.S. application Ser. No. 08/440,817 filed May 15, 1995; U.S. application Ser. No. 08/668,236 filed Jun. 14, 1996 which claimed the benefit of U.S. provisional application 60/000,275 filed Jun. 16, 1995; U.S. application Ser. No. 08/672,741 filed Jun. 28, 1996 which claimed the benefit of U.S. provisional application 60/000,744 filed Jun. 30, 1995; U.S. application Ser. No. 08/688,870 filed Aug. 31, 1996 which claimed the benefit of U.S. provisional application 60/001,754 filed Aug. 1, 1995; U.S. application Ser. No. 08/725,587 filed Oct. 3, 1996 which claimed the benefit of U.S. provisional application 60/004,697 filed Oct. 3, 1995; U.S. application Ser. No. 08/725,863 filed Oct. 4, 1996 which claimed the benefit of U.S. provisional application 60/005,175 filed Oct. 2, 1995; U.S. application Ser. No. 08/724,751 filed Oct. 2, 1996 which claimed the benefit of U.S. provisional application 60/004,810 filed Oct. 2, 1995; U.S. application Ser. No. 08/992,868 filed Dec. 11, 1997 which claimed the benefit of U.S. provisional application 60/032,838 filed Dec. 13, 1996 and is a continuation-in-part of U.S. application Ser. No. 08/734,050 filed Oct. 18, 1996 which claimed the benefit of U.S. provisional application 60/006,111 filed Oct. 24, 1995; U.S. application Ser. No. 08/723,972 filed Sep. 27, 1996 which claimed the benefit of U.S. provisional application 60/004,490 filed Sep. 29, 1995; U.S. application Ser. No. 08/725,029 filed Oct. 2, 1996 which claimed the benefit of U.S. provisional application 60/004,672 filed Oct. 2, 1995; U.S. application Ser. No. 08/722,922 filed Sep. 27, 1996 which claimed the benefit of U.S. provisional application 60/005,526 filed Sep. 29, 1995; U.S. application Ser. No. 08/725,693 filed Sep. 30, 1996 which claimed the benefit of U.S. provisional application 60/004,809 filed Oct. 2, 1995; U.S. application Ser. No. 08/731,034 filed Oct. 2, 1996 which claimed the benefit of U.S. provisional application 60/004,674 filed Oct. 2, 1995; U.S. application Ser. No. 08/706,766 filed Sep. 27, 1996 which claimed the benefit of U.S. provisional application 60/004,676 filed Oct. 2, 1995; U.S. application Ser. No. 08/727,870 filed Oct. 9, 1996 which claimed the benefit of U.S. provisional application 60/005,023 filed Oct. 10, 1995; U.S. application Ser. No. 08/726,759 filed Oct. 9, 1996 which claimed the benefit of U.S. provisional application 60/005,197 filed Oct. 10, 1995; U.S. application Ser. No. 08/727,737 filed Oct. 8, 1996 which claimed the benefit of U.S. provisional application 60/005,008 filed Oct. 10, 1995; U.S. application Ser. No. 08/740,370 filed Oct. 25, 1996 which claimed the benefit of U.S. provisional application 60/007,127 filed Oct. 26, 1995; U.S. application Ser. No. 08/748,106 filed Nov. 8, 1996 which claimed the benefit of U.S. provisional application 60/006,430 filed Nov. 10, 1995; U.S. application Ser. No. 08/730,774 filed Oct. 16, 1996 which claimed the benefit of U.S. provisional application 60/008,181 filed Oct. 30, 1995; U.S. application Ser. No. 08/749,515 filed Nov. 15, 1996 which claimed the benefit of U.S. provisional application 60/006,810 filed Nov. 15, 1995; U.S. application Ser. No. 08/755,337 filed Nov. 22, 1996 which claimed the benefit of U.S. provisional application 60/007,441 filed Nov. 22, 1995; U.S. application Ser. No. 08/755,524 filed Nov. 22, 1996 which claimed the benefit of U.S. provisional application 60/007,495 filed Nov. 22, 1995; U.S. application Ser. No. 08/727,898 filed Oct. 9, 1996 which claimed the benefit of U.S. provisional application 60/005,018 filed Oct. 10, 1995; U.S. application Ser. No. 08/736,641 filed Oct. 24, 1996 which claimed the benefit of U.S. provisional application 60/007,135 filed Oct. 31, 1995; U.S. application Ser. No. 08/706,765 filed Sep. 27, 1996 which claimed the benefit of U.S. provisional application 60/004,808 filed Sep. 29, 1995; U.S. application Ser. No. 08/720,914 filed Oct. 3, 1996 which claimed the benefit of U.S. provisional application 60/004,703 filed Oct. 3, 1995; U.S. application Ser. No. 08/725,872 filed Oct. 4, 1996 which claimed the benefit of U.S. provisional application 60/004,952 filed Oct. 4, 1995; U.S. application Ser. No. 08/725,106 filed Oct. 2, 1996 which claimed the benefit of U.S. provisional application 60/004,673 filed Oct. 2, 1995; U.S. application Ser. No. 08/725,595 filed Oct. 3, 1996 which claimed the benefit of U.S. provisional application 60/004,807 filed Oct. 3, 1995; U.S. application Ser. No. 08/992,332 filed Dec. 16, 1997 which claimed the benefit of U.S. provisional application 60/033,364 filed Dec. 17, 1996 and is a continuation-in-part of U.S. application Ser. No. 08/728,362 filed Oct. 9, 1996 which claimed the benefit of U.S. provisional application 60/005,019 filed Oct. 10, 1995; U.S. application Ser. No. 08/724,932 filed Oct. 2, 1996 which claimed the benefit of U.S. provisional application 60/004,669 filed Oct. 2, 1995; U.S. application Ser. No. 08/727,699 filed Oct. 3, 1996 which claimed the benefit of U.S. provisional application 60/004,704 filed Oct. 3, 1995; U.S. application Ser. No. 08/725,796 filed Oct. 3, 1996 which claimed the benefit of U.S. provisional application 60/004,696 filed Oct. 3, 1995; U.S. application Ser. No. 08/724,942 filed Oct. 2, 1996 which claimed the benefit of U.S. provisional application 60/004,675 filed Oct. 2, 1995; U.S. application Ser. No. 08/733,246 filed Oct. 2, 1996 which claimed the benefit of U.S. provisional application 60/004,677 filed Oct. 2, 1995; U.S. application Ser. No. 08/729,357 filed Oct. 16, 1996 which claimed the benefit of U.S. provisional application 60/006,031 filed Oct. 24, 1995; U.S. application Ser. No. 08/725,445 filed Oct. 4, 1996 which claimed the benefit of U.S. provisional application 60/004,955 filed Oct. 4, 1995; U.S. application Ser. No. 08/734,338 filed Oct. 21, 1996 which claimed the benefit of U.S. provisional application 60/007,078 filed Oct. 25, 1995; U.S. application Ser. No. 08/811,145 filed Oct. 24, 1996 which claimed the benefit of U.S. provisional application 60/005,855 filed Oct. 25, 1995; U.S. application Ser. No. 08/733,047 filed Oct. 15, 1996 which claimed the benefit of U.S. provisional application 60/008,004 filed Oct. 27, 1995; U.S. application Ser. No. 08/742,285 filed Oct. 24, 1996 which claimed the benefit of U.S. provisional application 60/006,105 filed Oct. 25, 1995; U.S. application Ser. No. 08/733,814 filed Oct. 18, 1996 which claimed the benefit of U.S. provisional application 60/005,864 filed Oct. 26, 1995; U.S. application Ser. No. 08/738,431 filed Oct. 24, 1996 which claimed the benefit of U.S. provisional application 60/005,854 filed Oct. 25, 1995; U.S. application Ser. No. 08/734,337 filed Oct. 21, 1996 which claimed the benefit of U.S. provisional application 60/009,862 filed Oct. 27, 1995; U.S. application Ser. No. 08/738,936 filed Oct. 24, 1996 which claimed the benefit of U.S. provisional application 60/007,199 filed Oct. 26, 1995; U.S. application Ser. No. 08/770,368 filed Nov. 27, 1996 which claimed the benefit of U.S. provisional applications 60/007,579 filed Nov. 29, 1995 and 60/008,542 filed Dec. 13, 1995; U.S. application Ser. No. 08/757,204 filed Nov. 27, 1996 which claimed the benefit of U.S. provisional applications 60/007,720 filed Nov. 30, 1995 and 60/008,793 filed Dec. 18, 1995; U.S. application Ser. No. 08/758,685 filed Nov. 27, 1996 which claimed the benefit of U.S. provisional application 60/007,611 filed Nov. 28, 1995; U.S. application Ser. No. 08/763,920 filed Dec. 11, 1996 which claimed the benefit of U.S. provisional application 60/008,794 filed Dec. 14, 1995; U.S. application Ser. No. 08/766,412 filed Dec. 12, 1996 which claimed the benefit of U.S. provisional application 60/008,605 filed Dec. 14, 1995; U.S. application Ser. No. 08/772,783 filed Dec. 23, 1996 which claimed the benefit of U.S. provisional application 60/009,447 filed Dec. 29, 1995; U.S. application Ser. No. 08/770,384 filed Dec. 12, 1996 which claimed the benefit of U.S. provisional applications 60/009,486 filed Dec. 15, 1995 and 60/009,132 filed Dec. 22, 1995; U.S. application Ser. No. 08/782,035 filed Dec. 20, 1996 which claimed the benefit of U.S. provisional application 60/011,774 filed Dec. 20, 1995; U.S. application Ser. No. 08/801,504 filed Dec. 19, 1996 which claimed the benefit of U.S. provisional application 60/009,710 filed Dec. 19, 1995; U.S. application Ser. No. 08/779,004 filed Jan. 3, 1997 which claimed the benefit of U.S. provisional application 60/009,671 filed Jan. 5, 1996; U.S. application Ser. No. 08/779,625 filed Jan. 17, 1997 which claimed the benefit of U.S. provisional application 60/010,158 filed Jan. 18, 1996; U.S. application Ser. No. 08/791,173 filed Jan. 30, 1997 which claimed the benefit of U.S. provisional application 60/011,001 filed Jan. 30, 1996; U.S. application Ser. No. 08/791,028 filed Jan. 29, 1997 which claimed the benefit of U.S. provisional application 60/010,777 filed Jan. 29, 1996; U.S. application Ser. No. 08/951,195 filed Oct. 1, 1997 which claimed the benefit of U.S. provisional application 60/027,319 filed Oct. 1, 1996 and is a continuation-in-part of U.S. application Ser. No. 08/792,739 filed on Jan. 24, 1997 which claimed the benefit of 60/011,138 filed Jan. 26, 1996; U.S. application Ser. No. 08/790,685 filed Jan. 29, 1997 which claimed the benefit of U.S. provisional application 60/011,132 filed Jan. 29, 1996; U.S. application Ser. No. 08/792,586 filed Jan. 30, 1997 which claimed the benefit of U.S. provisional applications 60/010,832 filed Jan. 30, 1996 and 60/010,812 filed Jan. 30, 1996; U.S. application Ser. No. 08/986,693 filed Dec. 5, 1997 which claimed the benefit of U.S. provisional application 60/032,151 filed Dec. 6, 1996 and is a continuation-in-part of U.S. applications Ser. No. 08/804,438, abandoned, filed on Feb. 21, 1997 which claimed the benefit of U.S. provisional application 60/012,049 filed Feb. 22, 1996 and Ser. No. 08/807,126 filed Jan. 31, 1997 which claimed the benefit of U.S. provisional application 60/011,010 filed Jan. 31, 1996; U.S. application Ser. No. 08/971,401 filed Nov. 14, 1997 which claimed the benefit of U.S. provisional application 60/131,419 filed Nov. 21, 1996 and is a continuation-in-part of Ser. No. 08/831,313 filed Feb. 21, 1997 which claimed the benefit of U.S. provisional application 60/012,084 filed Feb. 22, 1996; U.S. application Ser. No. 08/808,048 filed Feb. 27, 1997 which claimed the benefit of U.S. provisional application 60/012,357 filed Feb. 27, 1996; U.S. application Ser. No. 08/803,091 filed Feb. 20, 1997 which claimed the benefit of U.S. provisional application 60/012,213 filed Feb. 23, 1996; U.S. application Ser. No. 08/807,190 filed Feb. 27, 1997 which claimed the benefit of U.S. provisional application 60/012,367 filed Feb. 27, 1996; U.S. application Ser. No. 08/962,919 filed Oct. 23, 1997 which claimed the benefit of U.S. provisional applications 60/029,306 filed Oct. 25, 1996 and 60/036,403 filed Jan. 21, 1997 and is a continuation-in-part of U.S. application Ser. No. 08/806,593 filed Feb. 26, 1997 which claimed the benefit of 60/012,243 filed Feb. 26, 1996; U.S. application Ser. No. 08/808,443 filed Feb. 26, 1997 which claimed the benefit of U.S. provisional application 60/012,233 filed Feb. 26, 1996; U.S. application Ser. No. 08/806,796 filed Feb. 26, 1997 which claimed the benefit of U.S. provisional application 60/012,701 filed Feb. 26, 1996; U.S. application Ser. No. 08/941,869 filed Sep. 30, 1997 which claimed the benefit of U.S. provisional application 60/027,839 filed Oct. 1, 1996 and is a continuation-in-art of Ser. No. 08/999,941 filed Feb. 27, 1997 which claimed the benefit of 60/012,689 filed Feb. 27, 1996; U.S. application Ser. No. 08/808,904 filed Feb. 28, 1997 which claimed the benefit of U.S. provisional applications 60/012,698 filed Feb. 29, 1996, 60/013,365 filed Mar. 13, 1996 and 60/027,122 filed Sep. 27, 1996; U.S. application Ser. No. 08/810,326 filed Feb. 28, 1997 which claimed the benefit of U.S. provisional applications 60/0012,699 filed Feb. 29, 1996 and 60/015,173 filed Apr. 10, 1996; U.S. application Ser. No. 08/810,636 filed Feb. 28, 1997 which claimed the benefit of U.S. provisional application 60/012,706 filed Feb. 29, 1996; U.S. application Ser. No. 08/810,325 filed Feb. 28, 1997 which claimed the benefit of U.S. provisional applications 60/012,458 filed Feb. 28, 1996 and 60/013,001 filed Mar. 7, 1996; U.S. application Ser. No. 08/978,620 filed Nov. 25, 1997 which claimed the benefit of U.S. provisional application 60/032,151 filed Dec. 6, 1996 and is a continuation-in-part of U.S. application Ser. No. 08/812,505 filed Mar. 7, 1997 which claimed the benefit of U.S. provisional 60/013,529 filed Mar. 8, 1996; U.S. application Ser. No. 08/813,561 filed Mar. 7, 1997 which claimed the benefit of U.S. provisional application 60/013,062 filed Mar. 8, 1996; U.S. application Ser. No. 08/823,271 filed Mar. 21, 1997 which claimed the benefit of U.S. provisional application 60/015,513 filed Mar. 22, 1996; U.S. application Ser. No. 08/818,589 filed Mar. 18, 1997 which claimed the benefit of U.S. provisional application 60/015,377 filed Mar. 19, 1996; U.S. application Ser. No. 08/824,861 filed Mar. 26, 1997 which claimed the benefit of U.S. provisional application 60/013,543 filed Mar. 26, 1996; U.S. application Ser. No. 08/822,576 filed Mar. 18, 1997 which claimed the benefit of U.S. provisional application 60/018,106 filed Mar. 19, 1996; U.S. application Ser. No. 08/819,178 filed Mar. 17, 1997 which claimed the benefit of U.S. provisional application 60/013,991 filed Mar. 18, 1996; U.S. application Ser. No. 08/822,285 filed Mar. 20, 1997 which claimed the benefit of U.S. provisional application 60/013,696 filed Mar. 20, 1996; U.S. application Ser. No. 08/824,056 filed Mar. 21, 1997 which claimed the benefit of U.S. provisional application 60/013,786 filed Mar. 21, 1996; U.S. application Ser. No. 08/822,575 filed Mar. 19, 1997 which claimed the benefit of U.S. provisional application 60/013,744 filed Mar. 20, 1996; U.S. application Ser. No. 08/823,505 filed Mar. 25, 1997 which claimed the benefit of U.S. provisional application 60/014,174 filed Mar. 26, 1996; U.S. application Ser. No. 08/824,029 filed Mar. 25, 1997 which claimed the benefit of U.S. provisional application 60/014,010 filed Mar. 25, 1996; U.S. application Ser. No. 08/826,428 filed Mar. 25, 1997 which claimed the benefit of U.S. provisional application 60/014,305 filed Mar. 25, 1996; U.S. application Ser. No. 08/834,645 filed Apr. 10, 1997 which claimed the benefit of U.S. provisional application 60/017,170 filed Apr. 10, 1996; U.S. application Ser. No. 08/838,243 filed Apr. 16, 1997 which claimed the benefit of U.S. provisional application 60/015,724 filed Apr. 17, 1996; U.S. application Ser. No. 08/826,847 filed Apr. 10, 1997 which claimed the benefit of U.S. provisional application 60/015,533 filed Apr. 10, 1996; U.S. application Ser. No. 08/951,197 filed Oct. 1, 1997 which claimed the benefit of U.S. provisional application 60/027,249 filed Oct. 1, 1996 and is a continuation-in-part of U.S. application Ser. No. 08/826,438 filed Mar. 20, 1997 which claimed the benefit of U.S. provisional applications 60/016,145 filed Apr. 18, 1996 and 60/013,696 filed Mar. 20, 1996; U.S. application Ser. No. 08/835,705 filed Apr. 10, 1997 which claimed the benefit of U.S. provisional application 60/015,156 filed Apr. 10, 1996 and is a continuation-in-part of U.S. application Ser. No. 08/729,356, abandoned, filed Oct. 16, 1996 which claimed the benefit of U.S. provisional application 60/006,030 filed Oct. 24, 1995; U.S. application Ser. No. 08/837,998 filed Apr. 15, 1997 which claimed the benefit of U.S. provisional application 60/015,332 filed Apr. 15, 1996; U.S. application Ser. No. 08/858,195 filed Apr. 16, 1997 which claimed the benefit of U.S. provisional application 60/015,802 filed Apr. 17, 1996; U.S. application Ser. No. 08/844,690 filed Apr. 17, 1997 which claimed the benefit of U.S. provisional application 60/014,639 filed Apr. 17, 1996; U.S. application Ser. No. 08/845,717 filed Apr. 25, 1997 which claimed the benefit of U.S. provisional application 60/016,441 filed Apr. 25, 1996; U.S. application Ser. No. 08/839,389 filed Apr. 11, 1997 which claimed the benefit of U.S. provisional application 60/015,312 filed Apr. 12, 1996; U.S. application Ser. No. 08/951,198 filed Oct. 1, 1997 which claimed the benefit of U.S. provisional application 60/027,249 filed Oct. 1, 1996 and is a continuation-in-part of U.S. application Ser. No. 08/839,968, abandoned, filed Apr. 24, 1997 which claimed the benefit of U.S. provisional application 60/016,150 filed Apr. 24, 1996; U.S. application Ser. No. 08/843,950 filed Apr. 17, 1997 which claimed the benefit of U.S. provisional application 60/015,716 filed Apr. 17, 1996; U.S. application Ser. No. 08/827,938 filed Apr. 24, 1997 which claimed the benefit of U.S. provisional application 60/021,004 filed Apr. 25, 1996; U.S. application Ser. No. 08/845,255 filed Apr. 22, 1997 which claimed the benefit of U.S. provisional application 60/017,172 filed Apr. 22, 1996; U.S. application Ser. No. 08/842,978 filed Apr. 17, 1997 which claimed the benefit of U.S. provisional application 60/015,623 filed Apr. 19, 1996; U.S. application Ser. No. 08/845,751 filed Apr. 25, 1997 which claimed the benefit of U.S. provisional application 60/016,323 filed Apr. 26, 1996; U.S. application Ser. No. 08/845,723 filed Apr. 25, 1997 which claimed the benefit of U.S. provisional application 60/016,151 filed Apr. 25, 1996; U.S. application Ser. No. 08/846,104 filed Apr. 25, 1997 which claimed the benefit of U.S. provisional application 60/016,838 filed Apr. 26, 1996; U.S. application Ser. No. 08/839,045 filed Apr. 22, 1997 which claimed the benefit of U.S. provisional application 60/015,904 filed Apr. 22, 1996; U.S. application Ser. No. 08/878,503 filed Jun. 18, 1997 which claimed the benefit of U.S. provisional application 60/020,017 filed Jun. 20, 1996; U.S. application 09/107,910 filed Jun. 30, 1998 which is a continuation-in-part of U.S. application Ser. No. 08/845,721 filed Apr. 25, 1997 which claimed the benefit of U.S. provisional application 60/016,392 filed Apr. 26, 1996; U.S. application Ser. No. 08/847,659 filed Apr. 29, 1997 which claimed the benefit of U.S. provisional application 60/016,472 filed Apr. 29, 1996; U.S. application Ser. No. 08/863,878 filed May 27, 1997 which claimed the benefit of U.S. provisional application 60/018,682 filed May 29, 1996; U.S. application Ser. No. 08/865,594 filed May 20, 1997 which claimed the benefit of U.S. provisional applications 60/018,630 filed May 21, 1996 and 60/032,151 filed on Dec. 6, 1996; U.S. application Ser. No. 08/857,212 filed May 15, 1997 which claimed the benefit of U.S. provisional applications 60/018,139 filed May 16, 1996 and 60/066,336 filed Nov. 11, 1996; U.S. application Ser. No. 08/862,967 filed May 23, 1997 which claimed the benefit of U.S. provisional application 60/018,733 filed May 28, 1996; U.S. application Ser. No. 08/999,886 filed May 13, 1997 which claimed the benefit of U.S. provisional applications 60/017,555 filed May 13, 1996 and 60/031,667 filed Nov. 11, 1996; U.S. application Ser. No. 08/865,291 filed May 29, 1997 which claimed the benefit of U.S. provisional application 60/018,681 filed May 30, 1996; U.S. application Ser. No. 08/999,885 filed May 21, 1997 which claimed the benefit of U.S. provisional applications 60/018,650 filed May 21, 1996 and 60/023,338 filed Jun. 19, 1996; U.S. application Ser. No. 08/861,266 filed May 21, 1997 which claimed the benefit of U.S. provisional application 60/018,131 filed May 22, 1996; U.S. application Ser. No. 08/903,555 filed Jul. 31, 1997 which claimed the benefit of U.S. provisional application 60/023,308 filed Jul. 31, 1996 and is a continuation-in-part of U.S. application Ser. No. 08/862,178, abandoned, filed May 22, 1997 which claimed the benefit of U.S. provisional application 60/018,217 filed May 23, 1996; U.S. application Ser. No. 08/861,813 filed May 22, 1997 which claimed the benefit of U.S. provisional application 60/018,889 filed May 23, 1996; U.S. application Ser. No. 08/853,723 filed May 9, 1997 which claimed the benefit of U.S. provisional application 60/017,556 filed May 16, 1996; U.S. application Ser. No. 08/856,624 filed May 13, 1997 which claimed the benefit of U.S. provisional applications 60/017,190 filed May 13, 1996 and 60/027,100 filed Sep. 25, 1996; U.S. application Ser. No. 08/862,968 filed May 23, 1997 which claimed the benefit of U.S. provisional application 60/019,396 filed May 24, 1996; U.S. application Ser. No. 08/858,221 filed May 20, 1997 which claimed the benefit of U.S. provisional applications 60/018,629 filed May 21, 1996 and 60/020,372 filed Jun. 25, 1996; U.S. application Ser. No. 08/856,524 filed May 14, 1997 which claimed the benefit of U.S. provisional application 60/017,766 filed May 15, 1996; U.S. application Ser. No. 08/859,945 filed May 21, 1997 which claimed the benefit of U.S. provisional application 60/018,873 filed May 22, 1996; U.S. application Ser. No. 08/853,295 filed May 9, 1997 which claimed the benefit of U.S. provisional application 60/017,601 filed May 13, 1996; U.S. application Ser. No. 08/862,966 filed May 23, 1997 which claimed the benefit of U.S. provisional application 60/018,307 filed May 24, 1996; U.S. application Ser. No. 08/866,372 filed May 28, 1997 which claimed the benefit of U.S. provisional application 60/018,881 filed May 30, 1996; U.S. application Ser. No. 08/936,937 filed Sep. 24, 1997 which claimed the benefit of U.S. provisional application 60/027,107 filed Sep. 25, 1996 and is a continuation-in-part of U.S. application Ser. No. 08/730,775, abandoned, filed Oct. 16, 1996 which claimed the benefit of U.S. provisional 60/008,131 filed Oct. 31, 1995; U.S. application Ser. No. 08/878,504 filed Jun. 18, 1997 which claimed the benefit of U.S. provisional application 60/020,310 filed Jun. 24, 1996; U.S. application Ser. No. 08/885,220 filed Jun. 26, 1997 which claimed the benefit of U.S. provisional applications 60/020,591 filed Jun. 26, 1996 and 60/029,603 filed Oct. 23, 1996; U.S. application Ser. No. 08/881,589 filed Jun. 24, 1997 which claimed the benefit of U.S. provisional application 60/021,275 filed Jun. 25, 1996; U.S. application Ser. No. 08/878,507 filed Jun. 18, 1997 which claimed the benefit of U.S. provisional application 60/016,092 filed Jun. 19, 1996; U.S. application Ser. No. 08/879,204 filed Jun. 19, 1997 which claimed the benefit of U.S. provisional application 60/020,195 filed Jun. 20, 1996 and 60/027,234 filed Sep. 27, 1996; U.S. application Ser. No. 08/878,669 filed Jun. 19, 1997 which claimed the benefit of U.S. provisional applications 60/020,184 filed Jun. 21, 1996 and 60/023,298 filed Jul. 30, 1996; U.S. application Ser. No. 08/879,863 filed Jun. 19, 1997 which claimed the benefit of U.S. provisional applications 60/020,015 filed Jun. 21, 1996, 60/026,863 filed Sep. 30, 1996 and 60/035,192 filed Dec. 20, 1996; U.S. application Ser. No. 08/884,508 filed Jun. 27, 1997 which claimed the benefit of U.S. provisional application 60/020,690 filed Jun. 27, 1996; U.S. application Ser. No. 08/999,861 filed Jun. 27, 1997 which claimed the benefit of U.S. provisional applications 60/020,972 filed Jun. 28, 1996 and 60/028,852 filed Oct. 23, 1996; U.S. application Ser. No. 08/880,314 filed Jun. 23, 1997 which claimed the benefit of U.S. provisional applications 60/020,415 filed Jun. 24, 1996 and 60/025,817 filed Sep. 5, 1996; U.S. application Ser. No. 08/901,904 filed Jul. 28, 1997 which claimed the benefit of U.S. provisional applications 60/023,288 filed Jul. 29, 1996 and 60/033,302 filed Dec. 5, 1996; U.S. application Ser. No. 08/903,473 filed Jul. 3 1997 which claimed the benefit of U.S. provisional application 60/023,339 filed Jul. 30, 1996; U.S. application Ser. No. 08/903,474 filed Jul. 30, 1997 which claimed the benefit of U.S. provisional applications 60/023,339 filed Jul. 30, 1996 and 60/025,817 filed Sep. 5, 1996; U.S. application Ser. No. 08/900,106 filed Jul. 24, 1997 which claimed the benefit of U.S. provisional application 60/022,871 filed Jul. 24, 1996; U.S. application Ser. No. 08/901,903 filed Jul. 28, 1997 which claimed the benefit of U.S. provisional application 60/022,071 filed Jul. 29, 1996; U.S. application Ser. No. 08/903,802 filed Jul. 31, 1997 which claimed the benefit of U.S. provisional application 60/023,308 filed Jul. 31, 1996; U.S. application Ser. No. 08/905,881 filed Aug. 1, 1997 which claimed the benefit of U.S. provisional application 60/025,204 filed Aug. 1, 1996; U.S. application Ser. No. 08/900,522 filed Jul. 25, 1997 which claimed the benefit of U.S. provisional application 60/022,667 filed Jul. 26, 1996; U.S. application Ser. No. 08/900,077 filed Jul. 24, 1997 which claimed the benefit of U.S. provisional application 60/022,813 filed Jul. 26, 1996; U.S. application Ser. No. 08/905,885 filed Aug. 1, 1997 which claimed the benefit of U.S. provisional application 60/026,554 filed Aug. 1, 1996; U.S. application Ser. No. 08/902,620 filed Jul. 29, 1997 which claimed the benefit of U.S. provisional application 60/024,732 filed Jul. 29, 1996; U.S. application Ser. No. 08/899,858 filed Jul. 23, 1997 which claimed the benefit of U.S. provisional application 60/023,277 filed Jul. 24, 1996; U.S. application Ser. No. 08/903,214 filed Jul. 22, 1997 which claimed the benefit of U.S. provisional application 60/023,378 filed Jul. 23, 1996; U.S. application Ser. No. 08/899,451 filed Jul. 23, 1997 which claimed the benefit of U.S. provisional application 60/022,870 filed Jul. 24, 1996; U.S. application Ser. No. 08/900,107 filed Jul. 24, 1997 which claimed the benefit of U.S. provisional application 60/023,278 filed Jul. 25, 1996; U.S. application Ser. No. 08/900,076 filed Jul. 24, 1997 which claimed the benefit of U.S. provisional application 60/023,300 filed Jul. 26, 1996; U.S. application Ser. No. 08/902,618 filed Jul. 28, 1997 which claimed the benefit of U.S. provisional application 60/025,143 filed Jul. 29, 1996; U.S. application Ser. No. 08/903,517 filed Aug. 1, 1997 which claimed the benefit of U.S. provisional application 60/024,469 filed Aug. 1, 1996; U.S. application Ser. No. 08/903,471 filed Jul. 30, 1997 which claimed the benefit of U.S. provisional application 60/025,478 filed Jul. 31, 1996; U.S. application Ser. No. 08/901,455 filed Jul. 28, 1997 which claimed the benefit of U.S. provisional application 60/023,323 filed Jul. 30, 1996; U.S. application Ser. No. 09/041,895 filed Mar. 12, 1998 which is a continuation-in-part of U.S. application Ser. No. 08/905,278 filed Aug. 1, 1997 which claimed the benefit of U.S. provisional application 60/022,912 filed Aug. 1, 1996; U.S. application Ser. No. 08/901,902 filed Jul. 24, 1997 which claimed the benefit of U.S. provisional application 60/023,379 filed Jul. 26, 1996; U.S. application Ser. No. 08/903,472 filed Jul. 30, 1997 which claimed the benefit of U.S. provisional application 60/023,388 filed Jul. 30, 1996; U.S. application Ser. No. 08/903,556 filed Jul. 31, 1997 which claimed the benefit of U.S. provisional application 60/025,217 filed Aug. 22, 1996; U.S. application Ser. No. 08/917,045 filed Aug. 22, 1997 which claimed the benefit of U.S. provisional applications 60/025,216 filed Aug. 23, 1996 and 60/028,710 filed Nov. 20, 1996; U.S. application Ser. No. 08/918,910 filed Aug. 27, 1997 which claimed the benefit of U.S. provisional application 60/024,843 filed Aug. 28, 1996; U.S. application Ser. No. 08/918,671 filed Aug. 26, 1997 which claimed the benefit of U.S. provisional application 60/026,899 filed Aug. 28, 1996; U.S. application Ser. No. 08/918,181 filed Aug. 27, 1997 which claimed the benefit of U.S. provisional application 60/023,236 filed Aug. 28, 1996; U.S. application Ser. No. 08/917,046 filed Aug. 22, 1997 which claimed the benefit of U.S. provisional application 60/024,538 filed Aug. 22, 1996; U.S. application Ser. No. 08/920,758 filed Aug. 29, 1997 which claimed the benefit of U.S. provisional application 60/025,044 filed Aug. 30, 1996; U.S. application Ser. No. 08/929,307 filed Sep. 3, 1997 which claimed the benefit of U.S. provisional applications 60/025,375 filed Sep. 3, 1996 and 60/032,577 filed Dec. 4, 1996; U.S. application Ser. No. 08/923,861 filed Sep. 3, 1997 which claimed the benefit of U.S. provisional application 60/024,690 filed Sep. 5, 1996; U.S. application Ser. No. 08/917,047 filed Aug. 22, 1997 which claimed the benefit of U.S. provisional application 60/025,203 filed Aug. 23, 1996; U.S. application Ser. No. 08/923,871 filed Sep. 3, 1997 which claimed the benefit of U.S. provisional application 60/025,349 filed Sep. 3, 1996; U.S. application Ser. No. 08/923,903 filed Sep. 4, 1997 which claimed the benefit of U.S. provisional application 60/025,467 filed Sep. 5, 1996; U.S. application Ser. No. 08/918,182 filed Aug. 27, 1997 which claimed the benefit of U.S. provisional application 60/024,832 filed Aug. 28, 1996; U.S. application Ser. No. 08/918,972 filed Aug. 27, 1997 which claimed the benefit of U.S. provisional application 60/025,133 filed Aug. 30, 1996; U.S. application Ser. No. 08/922,314 filed Aug. 29, 1997 which claimed the benefit of U.S. provisional application 60/025,085 filed Aug. 30, 1996; U.S. application Ser. No. 08/916,651 filed Aug. 22, 1997 which claimed the benefit of U.S. provisional application 60/025,787 filed Aug. 22, 1996; U.S. application Ser. No. 08/937,142 filed Sep. 23, 1997 which claimed the benefit of U.S. provisional application 60/026,598 filed Sep. 24, 1996; U.S. application Ser. No. 08/937,141 filed Sep. 24, 1997 which claimed the benefit of U.S. provisional application 60/027,778 filed Sep. 27, 1996; U.S. application Ser. No. 08/940,864 filed Sep. 29, 1997 which claimed the benefit of U.S. provisional application 60/027,236 filed Sep. 30, 1996; U.S. application Ser. No. 08/936,026 filed Sep. 23, 1997 which claimed the benefit of U.S. provisional application 60/026,708 filed Sep. 25, 1996; U.S. application Ser. No. 08/943,978 filed Oct. 3, 1997 which claimed the benefit of U.S. provisional application 60/028,732 filed Oct. 4, 1996; U.S. application Ser. No. 08/940,032 filed Sep. 29, 1997 which claimed the benefit of U.S. provisional application 60/027,192 filed Sep. 30, 1996; U.S. application Ser. No. 08/943,980 filed Oct. 3, 1997 which claimed the benefit of U.S. provisional application 60/027,838 filed Oct. 4, 1996; U.S. application Ser. No. 08/951,196 filed Oct. 1, 1997 which claimed the benefit of U.S. provisional application 60/027,319 filed Oct. 1, 1996; U.S. application Ser. No. 08/941,479 filed Sep. 30, 1997 which claimed the benefit of U.S. provisional application 60/027,839 filed Oct. 1, 1996; U.S. application Ser. No. 08/941,884 filed Sep. 30, 1997 which claimed the benefit of U.S. provisional application 60/026,759 filed Oct. 3, 1996; U.S. application Ser. No. 08/943,979 filed Oct. 4, 1997 which claimed the benefit of U.S. provisional application 60/027,782 filed Oct. 4, 1996; U.S. application Ser. No. 08/956,502 filed Oct. 22, 1997 which claimed the benefit of U.S. provisional application 60/029,083 filed Oct. 23, 1996; U.S. application Ser. No. 08/951,203 filed Oct. 22, 1997 which claimed the benefit of U.S. provisional application 60/029,803 filed Oct. 23, 1996; U.S. application Ser. No. 09/032,271 filed Feb. 27, 1998 which claimed the benefit of U.S. provisional application 60/038,585 filed Mar. 5, 1997; U.S. application Ser. No. 08/959,395 filed Oct. 28, 1997 which claimed the benefit of U.S. provisional applications 60/030,755 filed Oct. 28, 1996 and 60/033,551 filed Dec. 20, 1996; U.S. application Ser. No. 08/960,746 filed Oct. 29, 1997 which claimed the benefit of U.S. provisional application 60/030,144 filed Oct. 30, 1996 and is a continuation-in-part of U.S. applications Ser. No. 08/826,847 filed Apr. 10, 1997 which claimed the benefit of U.S. provisional application 60/015,533 filed Apr. 10, 1996 and Ser. No. 08/755,524 filed Nov. 22, 1996 which claimed the benefit of U.S. provisional application 60/007,495 filed Nov. 22, 1995; U.S. application Ser. No. 08/959,394 filed Oct. 28, 1997 which claimed the benefit of U.S. provisional application 60/029,687 filed Oct. 31, 1996; U.S. application Ser. No. 08/957,777 filed Oct. 27, 1997 which claimed the benefit of U.S. provisional application 60/029,281 filed Oct. 28, 1996; U.S. application Ser. No. 08/957,941 filed Oct. 27, 1997 which claimed the benefit of U.S. provisional application 60/029,494 filed Oct. 29, 1996; U.S. application Ser. No. 08/958,558 filed Oct. 28, 1997 which claimed the benefit of U.S. provisional application 60/029,397 filed Oct. 30, 1996; U.S. application Ser. No. 08/958,783 filed Oct. 27, 1997 which claimed the benefit of U.S. provisional application 60/029,634 filed Oct. 28, 1996; U.S. application Ser. No. 08/960,745 filed Oct. 29, 1997 which claimed the benefit of U.S. provisional application 60/029,804 filed Oct. 31, 1996; U.S. application Ser. No. 08/961,524 filed Oct. 30, 1997 which claimed the benefit of U.S. provisional application 60/029,686 filed Oct. 31, 1996; U.S. application Ser. No. 08/961,525 filed Oct. 30, 1997 which claimed the benefit of U.S. provisional application 60/029,857 filed Oct. 31, 1996; U.S. application Ser. No. 08/960,741 filed Oct. 29, 1997 which claimed the benefit of U.S. provisional application 60/030,888 filed Oct. 31, 1996; U.S. application Ser. No. 08/961,526 filed Oct. 30, 1997 which claimed the benefit of U.S. provisional application 60/030,139 filed Oct. 31, 1996; U.S. application Ser. No. 08/959,663 filed Oct. 28, 1997 which claimed the benefit of U.S. provisional application 60/030,140 filed Oct. 31, 1996; U.S. application Ser. No. 08/936,936 filed Sep. 23, 1997 which claimed the benefit of U.S. provisional applications 60/026,708 filed Sep. 25, 1996 and 60/032,169 filed Dec. 4, 1996; U.S. application Ser. No. 08/976,538 filed Nov. 21, 1997 which claimed the benefit of U.S. provisional application 60/032,525 filed Dec. 5, 1996; U.S. application Ser. No. 08/966,494 filed Nov. 7, 1997 which claimed the benefit of U.S. provisional application 60/032,020 filed Nov. 22, 1996; U.S. application Ser. No. 08/984,567 filed Dec. 3, 1997 which claimed the benefit of U.S. provisional application 60/032,611 filed Dec. 4, 1996; U.S. application Ser. No. 08/971,400 filed Nov. 14, 1997 which claimed the benefit of U.S. provisional application 60/032,170 filed Dec. 4, 1996; U.S. application Ser. No. 08/966,493 filed Nov. 4, 1997 which claimed the benefit of U.S. provisional application 60/031,866 filed Nov. 25, 1996; U.S. application Ser. No. 08/974,756 filed Nov. 19, 1997 which claimed the benefit of U.S. provisional application 60/032,612 filed Dec. 4, 1996; U.S. application Ser. No. 08/992,625 filed Dec. 16, 1997 which claimed the benefit of U.S. provisional application 60/036,187 filed Dec. 18, 1996; U.S. application Ser. No. 08/994,065 filed Dec. 18, 1997 which claimed the benefit of U.S. provisional application 60/033,363 filed Dec. 18, 1996; U.S. application Ser. No. 08/903,469 filed Jul. 30, 1997 which claimed the benefit of U.S. provisional applications 60/033,755 filed Dec. 19, 1996 and 60/023,339 filed Jul. 30, 1996; U.S. application Ser. No. 08/993,402 filed Dec. 18, 1997 which claimed the benefit of U.S. provisional application 60/033,647 filed Dec. 19, 1996; U.S. application Ser. No. 08/992,829 filed Dec. 17, 1997 which claimed the benefit of U.S. provisional application 60/033,401 filed Dec. 18, 1996; U.S. application Ser. No. 09/016,866 filed Jan. 30, 1998 which claimed the benefit of U.S. provisional application 60/035,830 filed Jan. 30, 1997; U.S. application Ser. No. 08/984,691 filed Dec. 4, 1997 which claimed the benefit of U.S. provisional application 60/033,625 filed Dec. 16, 1996; U.S. application Ser. No. 08/993,774 filed Dec. 18, 1997 which claimed the benefit of U.S. provisional application 60/034,975 filed Dec. 20, 1996; U.S. application Ser. No. 08/994,066 filed Dec. 17, 1997 which claimed the benefit of U.S. provisional application 60/034,511 filed Dec. 19, 1996; U.S. application Ser. No. 09/012,473 filed Jan. 23, 1998 which claimed the benefit of U.S. provisional application 60/037,663 filed Jan. 27, 1997; U.S. application Ser. No. 09/014,442 filed Jan. 27, 1998 which claimed the benefit of U.S. provisional application 60/037,038 filed Jan. 27, 1997; U.S. application Ser. No. 09/015,081 filed Jan. 28, 1998 which claimed the benefit of U.S. provisional application 60/036,169 filed Jan. 28, 1997; U.S. application Ser. No. 09/010,765 filed Jan. 22, 1998 which claimed the benefit of U.S. provisional application 60/036,145 filed Jan. 22, 1997; U.S. application Ser. No. 09/013,311 filed Jan. 23, 1998 which claimed the benefit of U.S. provisional application 60/036,485 filed Jan. 27, 1997; U.S. application Ser. No. 09/012,923 filed Jan. 26, 1998 which claimed the benefit of U.S. provisional application 60/035,721 filed Jan. 28, 1997; U.S. application Ser. No. 09/014,441 filed Jan. 27, 1998 which claimed the benefit of U.S. provisional application 60/034,841 filed Jan. 27, 1997; U.S. application Ser. No. 09/015,080 filed Jan. 28, 1998 which claimed the benefit of U.S. provisional application 60/037,039 filed Jan. 28, 1997; U.S. application Ser. No. 09/012,922 filed Jan. 23, 1998 which claimed the benefit of U.S. provisional application 60/036,471 filed Jan. 27, 1997; U.S. application Ser. No. 09/010,153 filed Jan. 21, 1998 which claimed the benefit of U.S. provisional application 60/035,689 filed Jan. 21, 1997; U.S. application Ser. No. 09/010,638 filed Jan. 22, 1998 which claimed the benefit of U.S. provisional application 60/036,167 filed Jan. 22, 1997; U.S. application Ser. No. 08/923,902 filed Sep. 4, 1997 which claimed the benefit of U.S. provisional applications 60/036,279 filed Jan. 22, 1997 and 60/025,467 filed Sep. 5, 1996; U.S. application Ser. No. 09/016,884 filed Jan. 30, 1998 which claimed the benefit of U.S. provisional application 60/037,043 filed Jan. 30, 1997; U.S. application Ser. No. 09/013,812 filed Jan. 26, 1998 which claimed the benefit of U.S. provisional application 60/036,570 filed Jan. 29, 1997; U.S. application Ser. No. 09/021,033 filed Feb. 10, 1998 which claimed the benefit of U.S. provisional application 60/037,697 filed Feb. 11, 1997; U.S. application Ser. No. 09/021,095 filed Feb. 9, 1998 which claimed the benefit of U.S. provisional application 60/038,210 filed Feb. 11, 1997; U.S. application Ser. No. 09/021,031 filed Feb. 10, 1998 which claimed the benefit of U.S. provisional application 60/039,325 filed Feb. 13, 1997; U.S. application Ser. No. 09/022,355 filed Feb. 11, 1998 which claimed the benefit of U.S. provisional application 60/044,847 filed Feb. 13, 1997; U.S. application Ser. No. 09/021,702 filed Feb. 10, 1998 which claimed the benefit of U.S. provisional application 60/041,220 filed Feb. 12, 1997; U.S. application Ser. No. 09/036,591 filed Mar. 6, 1998 which claimed the benefit of U.S. provisional application 60/036,549 filed Mar. 6, 1997; U.S. application Ser. No. 09/035,169 filed Mar. 2, 1998 which claimed the benefit of U.S. provisional application 60/039,415 filed Mar. 5, 1997; U.S. application Ser. No. 09/031,483 filed Feb. 26, 1998 which claimed the benefit of U.S. provisional application 60/044,653 filed Mar. 4, 1997; U.S. application Ser. No. 09/035,173 filed Mar. 4, 1998 which claimed the benefit of U.S. provisional application 60/040,431 filed Mar. 5, 1997; U.S. application Ser. No. 09/035,172 filed Mar. 4, 1998 which claimed the benefit of U.S. provisional application 60/040,431 filed Mar. 5, 1997; U.S. application Ser. No. 09/034,981 filed Mar. 4, 1998 which claimed the benefit of U.S. provisional application 60/040,957 filed Mar. 7, 1997; U.S. application Ser. No. 09/035,171 filed Mar. 3, 1998 which claimed the benefit of U.S. provisional application 60/039,416 filed Mar. 5, 1997; U.S. application Ser. No. 09/035,170 filed Mar. 3, 1998 which claimed the benefit of U.S. provisional application 60/039,051 filed Mar. 6, 1997; U.S. application Ser. No. 09/036,309 filed Mar. 5, 1998 which claimed the benefit of U.S. provisional application 60/040,164 filed Mar. 7, 1997; U.S. application Ser. No. 09/021,032 filed Feb. 10, 1998 which claimed the benefit of U.S. provisional application 60/037,058 filed Feb. 12, 1997; U.S. application Ser. No. 09/021,700 filed Feb. 10, 1998 which claimed the benefit of U.S. provisional application 60/037,137 filed Feb. 13, 1997; U.S. application Ser. No. 09/036,589 filed Mar. 6, 1998 which claimed the benefit of U.S. provisional application 60/039,128 filed Mar. 6, 1997; U.S. application Ser. No. 09/041,720 filed Mar. 12, 1998 which claimed the benefit of U.S. provisional application 60/040,197 filed Mar. 14, 1997; U.S. application Ser. No. 09/049,638 filed Mar. 27, 1998 which claimed the benefit of U.S. provisional application 60/041,971 filed Mar. 27, 1997; U.S. application Ser. No. 09/045,574 filed Mar. 20, 1998 which claimed the benefit of U.S. provisional application 60/041,275 filed Mar. 21, 1997; U.S. application Ser. No. 09/042,629 filed Mar. 16, 1998 which claimed the benefit of U.S. provisional application 60/043,613 filed Mar. 18, 1997; U.S. application Ser. No. 09/036,310 filed Mar. 5, 1998 which claimed the benefit of U.S. provisional application 60/041,249 filed Mar. 19, 1997; U.S. application Ser. No. 09/041,893 filed Mar. 12, 1998 which claimed the benefit of U.S. provisional application 60/040,449 filed Mar. 14, 1997; U.S. application Ser. No. 09/041,894 filed Mar. 12, 1998 which claimed the benefit of U.S. provisional application 60/040,199 filed Mar. 14, 1997; U.S. application Ser. No. 09/049,497 filed Mar. 27, 1998 which claimed the benefit of U.S. provisional application 60/042,121 filed Mar. 28, 1997; U.S. application Ser. No. 09/044,767 filed Mar. 19, 1998 which claimed the benefit of U.S. provisional application 60/044,848 filed Mar. 20, 1997; U.S. application Ser. No. 09/040,265 filed Mar. 17, 1998 which claimed the benefit of U.S. provisional application 60/041,229 filed Mar. 24, 1997; U.S. application Ser. No. 09/047,925 filed Mar. 25, 1998 which claimed the benefit of U.S. provisional application 60/042,356 filed Mar. 25, 1997; U.S. application Ser. No. 09/042,630 filed Mar. 16, 1998 which claimed the benefit of U.S. provisional application 60/039,335 filed Mar. 17, 1997; U.S. application Ser. No. 09/044,001 filed Mar. 18, 1998 which claimed the benefit of U.S. provisional application 601041,239 filed Mar. 21, 1997; U.S. application Ser. No. 09/049,820 filed Mar. 27, 1998 which claimed the benefit of U.S. provisional application 60/041,733 filed Mar. 27, 1997; U.S. application Ser. No. 09/050,815 filed Mar. 30, 1998 which claimed the benefit of U.S. provisional application 60/042,467 filed Mar. 31, 1997; U.S. application Ser. No. 09/050,816 filed Mar. 30, 1998 which claimed the benefit of U.S. provisional application 60/043,982 filed Mar. 31, 1997; U.S. application Ser. No. 09/052,916 filed Mar. 31, 1998 which claimed the benefit of U.S. provisional application 60/042,281 filed Mar. 31, 1997; U.S. application Ser. No. 09/057,988 filed Apr. 9, 1998 which claimed the benefit of U.S. provisional application 60/043,256 filed Apr. 16, 1997; U.S. application Ser. No. 09/065,511 filed Apr. 23, 1998 which claimed the benefit of U.S. provisional application 60/044,939 filed Apr. 25, 1997; U.S. application Ser. No. 09/065,730 filed Apr. 24, 1998 which claimed the benefit of U.S. provisional application 60/045,098 filed Apr. 29, 1997; U.S. application Ser. No. 09/040,266 filed Mar. 17, 1998 which claimed the benefit of U.S. provisional application 60/045,648 filed Apr. 18, 1997; U.S. application Ser. No. 09/070,695 filed Apr. 30, 1998 which claimed the benefit of U.S. provisional application 60/045,349 filed May 1, 1997; U.S. application Ser. No. 09/050,817 filed Mar. 30, 1998 which claimed the benefit of U.S. provisional application 60/043,792 filed Apr. 11, 1997; U.S. application Ser. No. 09/066,646 filed Apr. 24, 1998 which claimed the benefit of U.S. provisional application 60/045,097 filed Apr. 29, 1997; U.S. application Ser. No. 09/056,942 filed Apr. 8, 1998 which claimed the benefit of U.S. provisional application 60/044,082 filed Apr. 16, 1997; U.S. application Ser. No. 09/062,736 filed Apr. 17, 1998 which claimed the benefit of U.S. provisional application 60/045,650 filed Apr. 18, 1997; U.S. application Ser. No. 09/052,990 filed Mar. 31, 1998 which claimed the benefit of U.S. provisional application 60/043,558 filed Apr. 11, 1997; U.S. application Ser. No. 09/065,521 filed Apr. 23, 1998 which claimed the benefit of U.S. provisional application 60/044,808 filed Apr. 24, 1997; U.S. application Ser. No. 09/066,970 filed Apr. 23, 1998 which claimed the benefit of U.S. provisional application 60/044,798 filed Apr. 24, 1997; U.S. application Ser. No. 09/070.693 filed Apr. 29, 1998 which claimed the benefit of U.S. provisional application 60/044,029 filed May 1, 1997; U.S. application Ser. No. 09/070,616 filed Apr. 30, 1998 which claimed the benefit of U.S. provisional application 60/045,636 filed May 2, 1997; U.S. application Ser. No. 09/070,694 filed Apr. 30, 1998 which claimed the benefit of U.S. provisional application 60/044,030 filed May 2, 1997; U.S. application Ser. No. 09/061,835 filed Apr. 16, 1998 which claimed the benefit of U.S. provisional application 60/045,647 filed Apr. 18, 1997; U.S. application Ser. No. 09/079,958 filed May 15, 1998 which claimed the benefit of U.S. provisional application 60/048,728 filed Jun. 4, 1997; U.S. application Ser. No. 09/074,999 filed May 8, 1998 which claimed the benefit of U.S. provisional application 60/048,431 filed May 29, 1997; U.S. application Ser. No. 09/073,078 filed May 5, 1998 which claimed the benefit of U.S. provisional application 60/047,803 filed May 28, 1997; U.S. application Ser. No. 09/073,079 filed May 5, 1998 which claimed the benefit of U.S. provisional application 60/047,802 filed May 28, 1997; U.S. application Ser. No. 09/076,667 filed May 12, 1998 which claimed the benefit of U.S. provisional application 60/048,002 filed May 29, 1997; U.S. application Ser. No. 09/075,782 filed May 11, 1998 which claimed the benefit of U.S. provisional application 60/046,624 filed May 15, 1997; U.S. application Ser. No. 09/070,697 filed Apr. 30, 1998 which claimed the benefit of U.S. provisional application 60/046,408 filed May 14, 1997; U.S. application Ser. No. 09/075,075 filed May 8, 1998 which claimed the benefit of U.S. provisional application 60/046,594 filed May 15, 1997; U.S. application Ser. No. 09/081,109 filed May 18, 1998 which claimed the benefit of U.S. provisional application 60/048,727 filed Jun. 4, 1997; U.S. application Ser. No. 09/075,126 filed May 8, 1998 which claimed the benefit of U.S. provisional application 60/048,726 filed Jun. 2, 1997; U.S. application Ser. No. 09/079,688 filed May 15, 1998 which claimed the benefit of U.S. provisional application 60/048,328 filed Jun. 2, 1997; U.S. application Ser. No. 09/082,405 filed May 20, 1998 which claimed the benefit of U.S. provisional application 60/048,978 filed Jun. 6, 1997; U.S. application Ser. No. 09/081,518 filed May 19, 1998 which claimed the benefit of U.S. provisional application 60/052,925 filed Jun. 5, 1997; U.S. application Ser. No. 09/080,161 filed May 15, 1998 which claimed the benefit of U.S. provisional application 60/048,721 filed Jun. 2, 1997; U.S. application Ser. No. 09/079,506 filed May 14, 1998 which claimed the benefit of U.S. provisional application 60/048,729 filed Jun. 4, 1997; U.S. application Ser. No. 09/089,078 filed Jun. 2, 1998 which claimed the benefit of U.S. provisional application 60/048,722 filed Jun. 2, 1997; U.S. application Ser. No. 09/083,895 filed May 21, 1998 which claimed the benefit of U.S. provisional application 60/048,977 filed Jun. 6, 1997; U.S. application Ser. No. 09/074,978 filed May 8, 1998 which claimed the benefit of U.S. provisional application 60/046,685 filed May 16, 1997; U.S. application Ser. No. 09/090,643 filed Jun. 4, 1998 which claimed the benefit of U.S. provisional application 60/048,979 filed Jun. 6, 1997; U.S. application Ser. No. 09/107,592 filed Jun. 30, 1998 which claimed the benefit of U.S. provisional application 60/052,751 filed Jul. 1, 1997; U.S. application Ser. No. 09/103,003 filed Jun. 24, 1998 which claimed the benefit of U.S. provisional application 60/051,491 filed Jul. 1, 1997; U.S. application Ser. No. 09/107,909 filed Jun. 30, 1998 which claimed the benefit of U.S. provisional application 60/051,733 filed Jul. 3, 1997; U.S. application Ser. No. 09/100,454 filed Jun. 19, 1998 which claimed the benefit of U.S. provisional application 60/051,749 filed Jul. 3, 1997; U.S. application Ser. No. 09/103,736 filed Jun. 24, 1998 which claimed the benefit of U.S. provisional application 60/051,621 filed Jul. 2, 1997; U.S. application Ser. No. 09/105,427 filed Jun. 26, 1998 which claimed the benefit of U.S. provisional application 60/051,363 filed Jun. 30, 1997; U.S. application Ser. No. 09/100,444 filed Jun. 19, 1998 which claimed the benefit of U.S. provisional application 60/051,493 filed Jun. 30, 1997; U.S. application Ser. No. 09/105,263 filed Jun. 26, 1998 which claimed the benefit of U.S. provisional application 60/051,461 filed Jul. 1, 1997; U.S. application Ser. No. 09/103,841 filed Jun. 24, 1998 which claimed the benefit of U.S. provisional application 60/051,591 filed Jul. 2, 1997; U.S. application Ser. No. 09/103,052 filed Jun. 24, 1998 which claimed the benefit of U.S. provisional application 60/051,611 filed Jul. 2, 1997; U.S. application Ser. No. 09/102,899 filed Jun. 23, 1998 which claimed the benefit of U.S. provisional application 60/051,570 filed Jul. 2, 1997; U.S. application Ser. No. 09/107,426 filed Jun. 30, 1998 which claimed the benefit of U.S. provisional application 60/051,750 filed Jul. 3, 1997; U.S. application Ser. No. 09/107,425 filed Jun. 30, 1998 which claimed the benefit of U.S. provisional application 60/051,751 filed Jul. 2, 1997; U.S. application Ser. No. 09/103,004 filed Jun. 24, 1998 which claimed the benefit of U.S. provisional application 60/051,492 filed Jul. 1, 1997; U.S. application Ser. No. 08/751,133 filed Nov. 15,1996 which claimed the benefit of U.S. provisional application 60/006,919 filed Nov. 17,1995; U.S. application Ser. No. 08/783,425 filed Jan. 10, 1997 which claimed the benefit of U.S. provisional application 60/009,753 filed Jan. 10, 1996; U.S. application Ser. No. 08/824,416 filed Mar. 26, 1997 which claimed the benefit of U.S. provisional application 60/014,176 filed Mar. 26, 1996; U.S. application Ser. No. 08/882,419 filed Jun. 25, 1997 which claimed the benefit of U.S. provisional application 60/020,581 filed Jun. 26, 1996; U.S. application Ser. No. 08/867,019 filed Jun. 3, 1997 which claimed the benefit of U.S. provisional application 60/021,003 filed Jun. 4, 1996; U.S. application Ser. No. 08/869,540 filed Jun. 3, 1997 which claimed the benefit of U.S. provisional application 60/018,911 filed Jun. 4, 1996; U.S. application Ser. No. 08/883,626 filed Jun. 26, 1997 which claimed the benefit of U.S. provisional application 60/021,995 filed Jun. 27, 1996; U.S. application Ser. No. 08/922,315 filed Aug. 29, 1997 which claimed the benefit of U.S. provisional applications 60/024,983 filed Aug. 29, 1996 and 60/031,923 filed Nov. 25, 1996; U.S. application Ser. No. 08/979,854 filed Nov. 20, 1997 which claimed the benefit of U.S. provisional application 60/031,834 filed Nov. 25, 1996 and is a continuation-in-part of Ser. No. 08/766,606 filed Dec. 12, 1996 which claimed the benefit of U.S. provisional application 60/010,338 filed Dec. 13, 1995; U.S. application Ser. No. 08/768,900 filed Dec. 13, 1996 which claimed the benefit of U.S. provisional application 60/008,732 filed Dec. 15, 1995 and is a continuation-in-part of Ser. No. 08/748,665 filed Nov. 13, 1996, abandoned, which claimed the benefit of U.S. provisional application 60/006,539 filed Nov. 13, 1995; U.S. application Ser. No. 09/096,307 filed Jun. 11, 1998 which claimed the benefit of U.S. provisional application 60/050,965 filed Jun. 13, 1997; U.S. application Ser. No. 09/292,568 filed Apr. 15, 1999 which claimed the benefit of U.S. provisional application 60/081,849 filed Apr. 15, 1998; U.S. application Ser. No. 09/293,657 filed Apr. 16, 1999 which claimed the benefit of U.S. provisional application 60/082,035 filed Apr. 16, 1998; U.S. application Ser. No. 09/293,655 filed Apr. 16, 1999 which claimed the benefit of U.S. provisional application 60/082,030 filed Apr. 16, 1998; U.S. application Ser. No. 09/092,505 filed Jun. 5, 1998 which claimed the benefit of U.S. provisional applications 60/048,976 filed Jun. 6, 1997 and 60/069,311 filed Dec. 4, 1997; U.S. application Ser. No. 09/092,504 filed Jun. 5, 1998 which claimed the benefit of U.S. provisional applications 60/049,364 filed Jun. 6, 1997 and 60/068,644 filed Dec. 23, 1997; U.S. application Ser. No. 09/093,822 filed Jun. 8, 1998 which claimed the benefit of U.S. provisional application 60/050,070 filed Jun. 12, 1997; U.S. application Ser. No. 09/094,079 filed Jun. 9, 1998 which claimed the benefit of U.S. provisional application 60/049,975 filed Jun. 13, 1997; U.S. application Ser. No. 09/112,430 filed Jul. 9, 1998 which claimed the benefit of U.S. provisional application 60/053,085 filed Jul. 9, 1997; U.S. application Ser. No. 09/112,577 filed Jul. 9, 1998 which claimed the benefit of U.S. provisional application 60/052,076 filed Jul. 9, 1997; U.S. application Ser. No. 09/113,753 filed Jul. 10, 1998 which claimed the benefit of U.S. provisional application 60/052,200 filed Jul. 10, 1997; U.S. application Ser. No. 09/114,041 filed Jul. 10, 1998 which claimed the benefit of U.S. provisional application 60/052,257 filed Jul. 10, 1997; U.S. application Ser. No. 09/114,061 filed Jul. 10, 1998 which claimed the benefit of U.S. provisional application 60/052,994 filed Jul. 10, 1997; U.S. application Ser. No. 09/112,371 filed Jul. 9, 1998 which claimed the benefit of U.S. provisional application 60/052,066 filed Jul. 9, 1997; U.S. application Ser. No. 09/126,377 filed Jul. 30, 1998 which claimed the benefit of U.S. provisional applications 60/054,913 filed Aug. 6, 1997 and 60/061,784 filed Oct. 10, 1997; U.S. application Ser. No. 09/128,841 filed Aug. 4, 1998 which claimed the benefit of U.S. provisional applications 60/055,031 filed Aug. 8, 1997 and 60/066,010 filed Nov. 5, 1997; U.S. application Ser. No. 09/131,380 filed Aug. 7, 1998 which claimed the benefit of U.S. provisional applications 60/055,705 filed Aug. 8, 1997 and 60/074,278 filed Feb. 10, 1998; U.S. application Ser. No. 09/128,833 filed Aug. 4, 1998 which claimed the benefit of U.S. provisional application 60/055,095 filed Aug. 5, 1997; U.S. application Ser. No. 09/129,873 filed Aug. 5, 1998 which claimed the benefit of U.S. provisional applications 60/056,395 filed Aug. 6, 1997, 60/066,011 filed Nov. 5, 1997 and 60/061,782 filed Oct. 10, 1997; U.S. application Ser. No. 09/128,809 filed Aug. 4, 1998 which claimed the benefit of U.S. provisional applications 60/055,398 filed Aug. 6, 1997 and 60/077,768 filed Mar. 11, 1998; U.S. application Ser. No. 09/149,716 filed Sep. 8, 1998 which claimed the benefit of U.S. provisional applications 60/058,540 filed Sep. 9, 1997 and 60/069,690 filed Dec. 11, 1997; U.S. application Ser. No. 09/145,501 filed Sep. 1, 1998 which claimed the benefit of U.S. provisional applications 60/058,853 filed Sep. 11, 1997, 60/061,617 filed Oct. 8, 1997 and 60/069,691 filed Dec. 12, 1997; U.S. application Ser. No. 09/145,340 filed Sep. 1, 1998 which claimed the benefit of U.S. provisional application 60/058,922 filed Sep. 11, 1997; U.S. application Ser. No. 09/151,199 filed Sep. 10, 1998 which claimed the benefit of U.S. provisional applications 60/058,471 filed Sep. 9, 1997 and 60/069,449 filed Dec. 11, 1997; U.S. application Ser. No. 09/152.050 filed Sep. 9, 1998 which claimed the benefit of U.S. provisional applications 60/058,979 filed Sep. 12, 1997 and 60/064,580 filed Nov. 4, 1997; U.S. application Ser. No. 09/148,482 filed Sep. 4, 1998 which claimed the benefit of U.S. provisional applications 60/058,921 filed Sep. 12, 1997 and 60/084,132 filed May 1, 1998; U.S. application Ser. No. 09/149,798 filed Sep. 8, 1998 which claimed the benefit of U.S. provisional applications 60/058,459 filed Sep. 8, 1997 and 60/058,382 filed Sep. 8, 1997; U.S. application Ser. No. 09/148,483 filed Sep. 4, 1998 which claimed the benefit of U.S. provisional application 60/058,920 filed Sep. 11, 1997; U.S. application Ser. No. 09/167,461 filed Oct. 7, 1998 which claimed the benefit of U.S. provisional applications 60/062,949 filed Oct. 8, 1997 and 60/078,895 filed Mar. 13, 1998; U.S. application Ser. No. 09/166,560 filed Oct. 5, 1998 which claimed the benefit of U.S. provisional applications 60/061,478 filed Oct. 8, 1997 and 60/080,843 filed Apr. 6, 1998; U.S. application Ser. No. 09/169,662 filed Oct. 8, 1998 which claimed the benefit of U.S. provisional application 60/063,478 filed Oct. 10, 1997; U.S. application Ser. No. 09/166,699 filed Oct. 5, 1998 which claimed the benefit of U.S. provisional application 60/061,479 filed Oct. 8, 1997; U.S. application Ser. No. 09/187,860 filed Nov. 6, 1998 which claimed the benefit of U.S. provisional applications 60/064,907 filed Nov. 7, 1997 and 60/077,871 filed Mar. 12, 1998; U.S. application Ser. No. 09/209,059 filed Dec. 10, 1998 which claimed the benefit of U.S. provisional applications 60/069,654 filed Dec. 11, 1997; U.S. application Ser. No. 09/229,412 filed Jan. 11, 1999 which claimed the benefit of U.S. provisional applications 60/071,763 filed Jan. 16, 1998 and 60/071,729 filed Jan. 16, 1998; U.S. application Ser. No. 09/231,945 filed Jan. 12, 1999 which claimed the benefit of U.S. provisional applications 60/071,762 filed Jan. 16, 1998 and 60/071,764 filed Jan. 16, 1998; U.S. application Ser. No. 09/231,925 filed Jan. 14, 1999 which claimed the benefit of U.S. provisional applications 60/071,761 filed Jan. 16, 1998 and 60/074,279 filed Feb. 10, 1998; U.S. application Ser. No. 09/250,003 filed Feb. 10, 1999 which claimed the benefit of U.S. provisional application 60/074,364 filed Feb. 12, 1998; U.S. application Ser. No. 09/ 250,002 filed Feb. 10, 1999 which claimed the benefit of U.S. provisional application 60/074,363 filed Feb. 11, 1998; U.S. application Ser. No. 09/250,152 filed Feb. 11, 1999 which claimed the benefit of U.S. provisional applications 60/074,786 filed Feb. 12, 1998 and 60/074,592 filed Feb. 12, 1998; U.S. application Ser. No. 09/251,260 filed Feb. 12, 1999 which claimed the benefit of U.S. provisional applications 60/074,723 filed Feb. 13, 1998 and 60/074,724 filed Feb. 13, 1998; U.S. application Ser. No. 09/264,807 filed Mar. 9, 1999 which claimed the benefit of U.S. provisional application 60/077,769 filed Mar. 11, 1998; U.S. application Ser. No. 09/266,620 filed Mar. 9, 1999 which claimed the benefit of U.S. provisional applications 60/077,440 filed Mar. 10, 1998 and 60/081,067 filed Apr. 8, 1998; U.S. application Ser. No. 09/288,687 filed Apr. 6, 1999 which claimed the benefit of U.S. provisional applications 60/080,842 filed Apr. 6, 1998 and 60/080,942 filed Apr. 7, 1998; U.S. application Ser. No. 09/288,001 filed Apr. 7, 1999 which claimed the benefit of U.S. provisional application 60/081,215 filed Apr. 9, 1998; U.S. application Ser. No. 09/292,074 filed Apr. 14, 1999 which claimed the benefit of U.S. provisional application 60/084,313 filed May 5, 1998; U.S. application Ser. No. 09/294,086 filed Apr. 16, 1999 which claimed the benefit of U.S. provisional application 60/084,492 filed May 6, 1998; U.S. application Ser. No. 09/325,194 filed Jun. 3, 1999 which claimed the benefit of U.S. provisional application 60/089,037 filed Jun. 11, 1998; U.S. application Ser. No. 09/369,305 filed Aug. 5, 1999 which claimed the benefit of U.S. provisional application 60/096,070 filed Aug. 11, 1998; U.S. application Ser. No. 09/325,239 filed Jun. 3, 1999 which claimed the benefit of U.S. provisional application 60/088,762 filed Jun. 10, 1998; U.S. application Ser. No. 09/370,505 filed Aug. 9, 1999 which claimed the benefit of U.S. provisional application 60/096,463 filed Aug. 12, 1998; U.S. application Ser. No. 09/420,691 filed Oct. 15, 1999 which claimed the benefit of U.S. provisional application 60/106,063 filed Oct. 27, 1998 and is a continuation-in-part of U.S. application Ser. No. 09/114,053 filed Jul. 10,1998, abandoned, which claimed the benefit of U.S. provisional application 60/052,190 filed Jul. 10, 1997; U.S. application Ser. No. 09/390,960 filed Sep. 3, 1999 which claimed the benefit of U.S. provisional application 60/099,523 filed Sep. 8, 1998; U.S. application Ser. No. 09/393,027 filed Sep. 9, 1999 which claimed the benefit of U.S. provisional application 60/100,256 filed Sep. 11, 1998; U.S. application Ser. No. 09/391,923 filed Sep. 8, 1999 which claimed the benefit of U.S. provisional application 60/099,696 filed Sep. 9, 1998 and is a continuation-in-part of U.S. application Ser. No. 09/209,059 filed Dec. 10,1998, which claimed the benefit of U.S. provisional application 60/069,654 filed Dec. 11, 1997; U.S. application Ser. No. 09/478,821 filed Jan. 10, 2000 which claimed the benefit of U.S. provisional application 60/115,695 filed Jan. 11, 1999 and is a continuation-in-part of U.S. application Ser. No. 09/112,430 filed Jul. 9,1998 which claimed the benefit of U.S. provisional application 60/053,085 filed Jul. 9, 1997; U.S. application Ser. No. 09/482,965 filed Jan. 13, 2000 which claimed the benefit of U.S. provisional applications 60/116,183 filed Jan. 14, 1999 and 60/119,801 filed Feb. 11, 1999 and is a continuation-in-part of U.S. applications Ser. No. 09/114,061 filed Jul. 10,1998 which claimed the benefit of U.S. provisional application 60/052,994 filed Jul. 10, 1997 and Ser. No. 09/151,199 filed Sep. 10, 1998 which claimed the benefit of U.S. provisional applications 60/058,471 filed Sep. 9, 1997 and 60/069,449 filed Dec. 11, 1997; U.S. application Ser. No. 09/411,077 filed Oct. 4, 1999 which claimed the benefit of U.S. provisional applications 60/103,748 filed Oct. 9, 1998 and 60/111,900 filed Dec. 10, 1998; U.S. application Ser. No. 09/452,747 filed Dec. 1, 1999 which claimed the benefit of U.S. provisional application 60/111,910 filed Dec. 10, 1998; U.S. application Ser. No. 09/453,704 filed Dec. 13, 1999 which claimed the benefit of U.S. provisional application 60/111,751 filed Dec. 10, 1998; U.S. application Ser. No. 09/500,782 filed Feb. 8, 2000 which claimed the benefit of U.S. provisional application 60/119,705 filed Feb. 11, 1999; U.S. application Ser. No. 09/500,900 filed Feb. 9, 2000 which claimed the benefit of U.S. provisional application 60/120,015 filed Feb. 11, 1999; U.S. application Ser. No. 09/ (Attorney Docket PZ-0111 US) filed Mar. 8, 2000 which claimed the benefit of U.S. provisional applications 60/123,525 filed Mar. 8, 1999, 60/123,344 filed Mar. 8, 1999, 60/128,425 filed Apr. 8, 1999 and 60/131,614 filed Apr. 29, 1999.

This application claims the benefit of U.S. provisional applications 60/128,215 filed Apr. 7, 1999, 60/128,412 filed Apr. 8, 1999, 60/131,614 filed Apr. 29, 1999, 60/133,048 filed May 7, 1999, 60/133,875 filed May 12, 1999, 60/132,523 filed May 4, 1999, 60/132,945 filed May 6, 1999, 60/132,254 filed May 3, 1999, 60/137,398 filed Jun. 3, 1999, 60/137,499 filed Jun. 4, 1999, 60/141,230 filed Jun. 29, 1999, 60/141,231 filed Jun. 29, 1999, 60/142,895 filed Jul. 8, 1999, 60/142,699 filed Jul. 7, 1999, 60/142,605 filed Jul. 7, 1999, 60/140,135 filed Jun. 18, 1999, 60/141,584 filed Jun. 29, 1999, 60/141,232 filed Jun. 29, 1999, 60/148,204 filed Aug. 10, 1999, 60/148,003 filed Aug. 10, 1999, 60/148,463 filed Aug. 12, 1999, 60/147,475 filed Aug. 5, 1999, 60/147,465 filed Aug. 6, 1999, 60/158,260 filed Oct. 6, 1999, 60/156,963 filed Sep. 30, 1999, 60/158,258 filed Oct. 6, 1999, 60/158,347 filed Oct. 7, 1999, 60/158,346 filed Oct. 7, 1999, 60/158,345 filed Oct. 7, 1999, 60/164,461 filed Nov. 10, 1999, 60/164,538 filed Nov. 10, 1999, 60/164,443 filed Nov. 9, 1999, 60/164,845 filed Nov. 10, 1999, 60/164,378 filed Nov. 9, 1999, 60/164,539 filed Nov. 10, 1999, 60/169,400 filed Dec. 6, 1999, 60/169,402 filed Dec. 6, 1999, 60/169,536 filed Dec. 7, 1999, 60/169,403 filed Dec. 6, 1999, and 60/169,401 filed Dec. 6, 1999.

### FIELD OF THE INVENTION

The present invention relates to polynucleotides encoding or regulating electron transfer molecules. These mammalian polynucleotides may be used in the diagnosis, study, prevention and treatment of disease.

### BACKGROUND OF THE INVENTION

The mitochondrial electron transport (or respiratory) chain is a series of oxidoreductase-type enzyme complexes in the mitochondrial membrane that is responsible for the transport of electrons from NADPH through a series of redox centers within these complexes to oxygen, and the coupling of this oxidation to the synthesis of ATP (oxidative phosphorylation). ATP then provides the primary source of energy for driving a cell's many energy-requiring reactions. The key complexes in the respiratory chain are NADH:ubiquinone oxidoreductase (complex I), succinate:ubiquinone oxidoreductase (complex II), cytochrome c₁-b oxidoreductase (complex III), cytochrome c oxidase (complex IV), and ATP synthase (complex V) (Alberts. et al. (1994) Molecular Biology of the Cell, Garland Publishing, Inc., New York, N.Y., p. 677-678). All of these complexes are located on the inner matrix side of the mitochondrial membrane except complex II, which is on the cytosolic side. Complex II transports electrons generated in the citric acid cycle to the respiratory chain. The electrons generated by oxidation of succinate to fumarate in the citric acid cycle are transferred through electron carriers in complex II to membrane bound ubiquinone (coenzyme Q).

Electron transport through these complexes is mediated by a series of electron transfer proteins, most of which contain prosthetic groups such as flavins, heme, iron-sulfur clusters (FeS), or copper, bound to inner membrane proteins. Adrenodoxin, for example, is an FeS protein specific for adrenal gland tissue that forms a complex with NADPH:adrenodoxin reductase and cytochrome p450 in the conversion of cholesterol to pregnenlone (Pikuleva et at. (1999) J Biol Chem 274:2045-2052). Cytochromes contain a heme prosthetic group, a porphyrin ring containing a tightly bound iron atom. The iron atom serves as the actual electron carrier by changing from the ferric to the ferrous state when accepting an electron.

Cytochromes are classified into subgroups a, b, and c, according to distinctive absorption spectra conferred on them by differences in their interactions with the heme group. Cytochromes a, a₃, b₅₆₂, b₅₆₆, c, and c₁ are all components of the mammalian mitochondrial membrane respiratory chain involved in oxidative phosphorylation. Cyt b5 exists in both a membrane-bound form in mitochondria and endoplasmic reticulum and a soluble form in erythrocytes. The membrane-bound form has been linked with lipid and drug metabolism, and with NADPH-linked hydroxylation reactions (De Sylsvestris et al. (1995) FEBS Lett 370:69-74; Ozols (1989) Biochim et Biophys Acta 997:121-30).

Other electron transfer proteins include peroxiredoxin, a protein that serves as an antioxidant to remove cellular peroxides (Sarafian, (1999) J Neurosci Res 56:206-212); thioredoxin, an antioxidant that reduces hydrogen peroxide, scavenges free radicals, and protects cells against oxidative stress (Lee et al. (1998) J Biol Chem 273:19160-19166); and glutaredoxin, a glutathione-dependent hydrogen donor for ribonucleotide reductase and for glutathione reductase (Padilla, et al.(1995) Eur J Biochem 15:227:27-34).

### Diseases and Disorders Related to Expression of Electron Transfer Molecules

Mitochondrial dysfunction leads to impaired calcium buffering, generation of free radicals that may participate in deleterious intracellular and extracellular processes, changes in mitochondrial permeability and oxidative damage which is observed in several neurodegenerative diseases. Neurodegenerative diseases linked to mitochondrial dysfunction include some forms of Alzheimer's disease, Friedreich's ataxia, familial amyotrophic lateral sclerosis, and Huntington's disease (Beal (1998) Biochim Biophys Acta 1366:211-213). The myocardium is heavily dependent on oxidative metabolism, consequently mitochondrial dysfunction often leads to heart disease (DiMauro and Hirano (1998) Curr Opin Cardiol 13:190-197). Mitochondria are implicated in disorders of cell proliferation, since they play an important role in a cell's decision to proliferate or self-destruct through apoptosis. The oncoprotein Bc1-2, for example, promotes cell proliferation by stabilizing mitochondrial membranes so that apoptosis signals are not released (Susin (1998) Biochim Biophys Acta 1366:151-165). Restricted expression of genes encoding peroxiredoxin in brain cells may contribute to selective vulnerability of these cells to a wide variety of neuropathologic conditions (Sarafian, supra).

In view of the structure and function of electron transfer molecules and the conditions, diseases, disorders and syndromes in which they play a role, it is advantageous to provide mammalian polynucleotides, in particular human and rat polynucleotides, and methods for using these polynucleotides, and the polypeptides that they encode, in the diagnosis, study, prevention, and treatment of such disorders.

### SUMMARY OF THE INVENTION

The invention provides mammalian polynucleotides, in particular human and rat polynucleotides, as presented in the Sequence Listing. These polynucleotides comprise regulatory elements or encode polypeptides or portions thereof identified as electron transfer molecules.

The invention provides a purified mammalian polynucleotide comprising a nucleic acid sequence selected from SEQ ID NOs: 1-217 1; a fragment of at least 18 consecutive nucleotides selected from SEQ ID NOs: 1-2171; and a complement of the polynucleotide or fragment. The invention also provides a composition which may be used on a substrate. The invention further provides a probe comprising a polynucleotide selected from SEQ ID NOs: 1-2171, a fragment thereof or complement thereof.

The present invention encompasses oligonucleotides, fragments of polynucleotides, and polynucleotides complementary to the polynucleotides listed in the Sequence Listing, and DNA molecules which may be used in the diagnosis, study, prevention and treatment of disorders in which electron transfer molecules play a role. Additionally these polynucleotides are used to produce a profile of gene activity or in methods to detect normal or altered gene expression. Such methods employ the mammalian polynucleotides disclosed herein or their derivatives.

The present invention provides a method for diagnosing disorders associated with the expression and function of electron transfer molecules. Such a method comprises providing a plurality of polynucleotides selected from sequences of the Sequence Listing wherein each of the polynucleotides is identified by an abnormal level of expression in an individual at risk compared to the level of expression in a normal individual, providing a sample, hybridizing the sample with the plurality of polynucleotides, and detecting hybridization complexes wherein the formation of hybridization complexes indicates the presence of a disorder.

The present invention also encompasses the use of gene therapy methods for the introduction of polynucleotides of the present invention into subjects with disorders associated with abnormal gene expression. The coding region of the polynucleotide sequence is used to express polypeptides that are underexpressed or absent in a subject, and a fragment of the polynucleotide that may be an RNA, a DNA molecule or a ribozyme is used to prevent transcription or inhibit translation of an mRNA that is overexpressed in a subject. The present invention contemplates the use of oligonucleotides or fragments of the polynucleotide or their complements as antisense molecules to control gene expression. For example, an antisense molecule may be designed to inhibit translation of mRNA and may be used therapeutically in the treatment of disorders associated with defective or abnormal expression of a polynucleotide.

As disclosed herein, the polynucleotides of the Sequence Listing are used to detect, screen, amplify, purify, or quantify identical or related genes, DNA molecules, or RNA molecules in a sample, cell, substrate or solution, or in diagnostic assays or kits. The invention contemplates the use of such polynucleotides to identify or to construct a coding sequence for a polypeptide or a portion thereof.

The invention also provides host cells and expression vectors comprising polynucleotides of the present invention and methods for the production of polypeptides they encode. Such methods comprise culturing the host cells under conditions for the expression of the polynucleotide and recovering the polypeptide from the cell culture.

The subject invention also comprises a purified polypeptide of which at least a portion is encoded by a polynucleotide selected from the polynucleotides listed in the Sequence Listing.

The subject invention also comprises a method for making an antibody that specifically binds the polypeptide by immunizing an animal with the polypeptide or a portion thereof under conditions to elicit an immunological response, collecting serum/blood from the immunized animal, combining the serum/blood with the polypeptide under conditions to allow specific binding between the polypeptide and the antibody, recovering the bound polypeptide, and separating the polypeptide from the antibody, thereby producing antibody that specifically binds the polypeptide. The subject invention further comprises a purified antibody that specifically binds to the polypeptide of which at least a portion is encoded by a polynucleotides selected from the Sequence Listing. Antibodies produced by this method are used for diagnostic purposes to detect the polypeptide to which it specifically binds or for therapeutic purposes to neutralize the activity of a polypeptide in conditions, diseases, and disorders associated with altered or abnormal expression of the polypeptide.

The subject invention also provides methods of using a purified polynucleotide or polypeptide to identify a ligand that specifically binds the polynucleotide or polypeptide. Such a method comprises providing a library or a plurality of molecules or compounds, combining the polynucleotide or polypeptide with each of the molecules or compounds under conditions to allow binding between the polynucleotide or polypeptide and the molecule or compound, and detecting binding to a molecule or compound, thereby identifying a ligand that specifically binds the polynucleotide or polypeptide. Similar methods use the polynucleotide or polypeptide to purify a ligand, molecule or compound from a sample.

The invention further provides a method for inserting a marker gene into the genomic DNA of a mammal to disrupt the expression of the natural nucleic acid. The invention also provides a method for using a polynucleotide to produce a mammalian model system, the method comprising constructing a vector containing the nucleic acid molecule selected from SEQ ID NOs: 1-2171; transforming the vector into an embryonic stem cell; selecting a transformed embryonic stem; microinjecting the transformed embryonic stem cell into a mammalian blastocyst, thereby forming a chimeric blastocyst; transferring the chimeric blastocyst into a pseudopregnant dam, wherein the dam gives birth to a chimeric offspring containing the nucleic acid molecule in its germ line; and breeding the chimeric mammal to produce a homozygous, mammalian model system.

### DESCRIPTION OF THE SEQUENCE LISTING AND TABLES

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The Sequence Listing is a compilation of polynucleotides obtained by sequencing clone inserts (isolates) of human and rat cDNA libraries. Each sequence is identified by a sequence identification number (SEQ ID NO) and by an Incyte identification number (INCYTE ID NO). Tables 1, 2 and 3 are compilations of information about the polynucleotides presented in the Sequence Listing.

TABLE 1 presents a filing history for each of the polynucleotides from human and rat cDNA libraries that have been identified as electron transfer molecules and are found in the Sequence Listing. The first column contains SEQ ID NO; the second column, the INCYTE ID NO; the third column, CLONE number; the fourth column, TEMPLATE number; and the fifth column, LIBRARY name. The sixth column contains Incyte DOCKET NO; original SEQ ID NO; and the application FILING DATE.

TABLE 2 describes nucleotide alignment of the human and rat polynucleotides presented in the Sequence Listing to the template sequence from which it was annotated. The first column contains SEQ ID NO, the second column, CLONE number; and the third column, TEMPLATE number. The fourth and fifth columns describe the exact position of each of the human and rat polynucleotides, START and STOP nucleotides, along the length of the template sequence.

TABLE 3 presents GenBank annotations for the human and rat polynucleotides of the Sequence Listing. The first column contains SEQ ID NO; the second column, INCYTE ID NO; the third column, GENBANK ID; and the fourth column, the GENBANK ANNOTATION.

### DETAILED DESCRIPTION OF THE INVENTION

### Definitions

Since the list of technical and scientific terms cannot be all encompassing, any undefined terms shall be construed to have the same meaning as is commonly understood by one of skill in the art to which this invention belongs. Furthermore, the singular forms “a”, “an”, and “the” include plural reference unless the context clearly dictates otherwise.

An “allele” or allelic polynucleotide is an alternative form of a given gene. Alleles result from a mutation in the nucleic acid sequence and produce altered mRNAs or polypeptides whose structure or function may or may not be altered. Any given gene may have none, one, or many allelic forms. Common mutational changes that give rise to alleles are generally ascribed to deletions, insertions, or substitutions of nucleotides. Each of these types of changes may occur alone, or in combination with the others, one or more times in a given sequence.

“Biological activity” refers to those structural, regulatory, or biochemical functions of a polypeptide that depend on amino acid sequence and molecular conformation, reaction with appropriate receptor or effector molecules, and ability to function in vitro or in vivo. Many enzymes are transported or stored in a “prepro” or “pro” form and activated by cleavage of their N-terminal stabilizing and/or signal sequence after transport to their effective cellular location.

“Disorders”, as used herein, refers to conditions, diseases, disorders, and syndromes, whether genetic, infectious, or environmental, that are associated with abnormal or altered expression of electron transfer molecules These disorders include a cell proliferative disorder, such as arteriosclerosis, atherosclerosis, mixed connective tissue disease (MCTD), psoriasis, primary thrombocythemia, and cancers such as adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an immune disorder such as inflammation, actinic keratosis, acquired immunodeficiency syndrome (AIDS), Addison's disease, adult respiratory distress syndrome, allergies, anemia, asthma, autoimmune thyroiditis, bronchitis, bursitis, cholecystitis, cirrhosis, contact dermatitis, Crohn's disease, diabetes mellitus, emphysema, gout, Graves' disease, hepatitis, hypereosinophilia, irritable bowel syndrome, multiple sclerosis, myasthenia gravis, myocardial or pericardial inflammation, osteoarthritis, osteoporosis, pancreatitis, rheumatoid arthritis, scleroderma, and ulcerative colitis; and a neuronal disorder such as akathesia, Alzheimer's disease, amnesia, amyotrophic lateral sclerosis, anxiety, hereditary ataxias, cerebral palsy, dementia, dermatomyositis, dystonias, Down's syndrome, epilepsy, ischemic cerebrovascular disease, cerebelloretinal hemangioblastomatosis, Huntington's disease, bacterial and viral meningitis, multiple sclerosis, muscular dystrophy, myasthenia gravis, cerebral neoplasms, neurofibromatosis, Parkinson's disease, Pick's disease, polymyositis, retinitis pigmentosa, schizophrenia, and stroke, particularly as they afflict humans. Such disorders may be studied in rats or mice as they naturally occur or as modeled in transgenic knockout or knockin animals.

“Deletion” refers to a change in either nucleotide or amino acid sequence in which one or more nucleotides or amino acid residues, respectively, are absent.

“Derivative” refers to a polynucleotide or a polypeptide that has been subjected to a chemical modification. Illustrative of such modifications would be replacement of a hydrogen by, for example, an acetyl, acyl, alkyl, amino, formyl, or morpholino group. Derivative polynucleotides may encode polypeptides that retain the essential biological characteristics (such as catalytic and regulatory domains) of naturally occurring polypeptides.

“Fragment” refers to at least 18 consecutive nucleotides of a polynucleotide of the Sequence Listing or its complement. A “unique” fragment refers to at least 18 consecutive nucleotides of a particular polynucleotide or its complement that is specific to a polynucleotide of the Sequence Listing and that under hybridization conditions would not detect related polynucleotides in which it does not appear.

“Homology” refers to sequence similarity between a reference sequence and at least a fragment of a polynucleotide or a portion of a polypeptide.

“Hybridization complex” refers to a complex between two polynucleotides by virtue of the formation of hydrogen bonds between purines and pyrimidines.

“Immunological activity” is the capability of the natural, recombinant, or synthetic polypeptide or portion thereof to induce in an animal a specific immune response that results in the production of antibodies.

“Insertion” or “addition” is a change in a polynucleotide or polypeptide by incorporation of one or more nucleotides or residues.

“Ligand” refers to any agent, molecule, or compound that binds specifically to a polynucleotide or polypeptide. Ligands stabilize or modulate the activity of polynucleotides or polypeptides of the invention and may be composed of at least one of the following: inorganic and organic substances including nucleic acids, proteins, carbohydrates, fats, and lipids.

“Linkers” are synthesized oligonucleotides that may be inserted into or added onto a polynucleotide of interest to create restriction endonuclease sites for ease in cloning the polynucleotide into various vectors. “Polylinkers” are engineered to include multiple restriction enzyme sites. Linkers and polylinkers provide for the use of enzymes that leave 5′ and 3′ overhangs (such as BamHI, EcoRI, PstI, and the like) or that provide a blunt end (such as EcoRV, SnaBI, StuI, and the like).

“Modulates” refers to any change in activity (increased or decreased; biological, chemical, or immunological) or lifespan resulting from specific binding between a molecule and a polynucleotide or polypeptide of the invention.

“Naturally occurring” refers to a polynucleotide or polypeptide that is found in nature.

“Oligonucleotide” or “oligomer” refers to a nucleotide sequence of at least about 15 nucleotides to as many as about 60 nucleotides, preferably about 18 to 30 nucleotides, and most preferably about 20 to 25 nucleotides that are used as a “primer” or “amplimer” in the polymerase chain reaction (PCR) or as an array element.

“Polynucleotide” refers to an oligonucleotide, nucleotide sequence, nucleic acid molecule, DNA molecule, or any fragment or complement thereof. It may be DNA or RNA of genomic or synthetic origin, double-stranded or single-stranded, coding and/or noncoding, an exon or an intron of a genomic DNA molecule, or combined with carbohydrate, lipids, protein or inorganic elements or substances. “Oligonucleotide” is substantially equivalent to the terms amplimer, primer, oligomer, element, target, and probe and is preferably single stranded.

“Polypeptide” refers to a protein or any portion thereof including an oligopeptide. A portion of a polypeptide generally retains biological or immunogenic characteristics of a native protein. An “oligopeptide” is an amino acid sequence of at least about 5 residues, more preferably 10 residues and most preferably about 15 residues that are immunogenic and are used as part of a fusion protein to produce an antibody.

“Portion” refers to at least six contiguous amino acids of a polypeptide encoded by a polynucleotide of the Sequence Listing. A portion may represent an amino acid sequence that is conserved among related proteins (e.g., a catalytic domain such as a kinase domain).

“Post-translational modification” of a polypeptide may involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a polynucleotide or a fragment thereof that hybridizes to a nucleic acid molecule in a sample or on a substrate. A probe is used to detect, amplify, or quantify cDNAs, endogenous genes, or transcript mRNAs by employing conventional, molecular biology techniques. As used herein, probes are the reporter molecule of hybridization reactions including Southern, northern, in situ, dot blot, array, and like technologies used to determine whether electron transfer molecule DNA or RNA is present.

“Purified” refers to polynucleotides, polypeptides, antibodies, and the like, that are isolated from at least one other component with which they are naturally associated.

“Regulatory sequences or regions” are noncoding and generally refer to “control elements” including, but not limited to, enhancers, promoters, suppressors, introns, and 5′ and 3′ untranslated regions of a gene. These sequences interact with cellular proteins to carry out replication, transcription, and translation. They may occur as boundary sequences or as spacer regions in between genomic exons. Such sequences function at the molecular level and, along with regulatory genes, are very important in cellular and organismal development, growth, differentiation, and aging processes.

“Reporter molecules” are chemical or biochemical moieties used for labeling a polynucleotide, a polypeptide, or an antibody. They include, but are not limited to, radionuclides, enzymes, substrates, cofactors, inhibitors, fluorescent agents, chromogenic agents, chemiluminescent agents, magnetic particles, and the like. Reporter molecules specifically bind, establish the presence of, and allow quantification of a particular polynucleotide, polypeptide, or antibody.

“Sample” is used herein in its broadest sense. A sample containing polynucleotides, polypeptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA in solution or bound to a substrate; a cell; a tissue; a tissue print; a fingerprint, skin or hair; and the like.

“Single nucleotide polymorphism” (SNP) refers to a change in at least one single nucleotide in a polynucleotide that occurs as a result of a substitution, insertion or deletion. The change may be conservative (purine for purine) or non-conservative (purine to pyrimidine) and may or may not result in a change in an encoded amino acid residue. Such changes may predispose an individual to a specific disease or condition.

“Specific binding” or “specifically binding” refers to the interaction between two molecules. In the case of a polynucleotide, specific binding may involve hydrogen bonding between sense and antisense strands or between one stand and a protein which affects its replication or transcription, intercalation of a molecule or compound into the major or minor groove of the DNA molecule, or interaction with at least one molecule which functions as a transcription factor, enhancer, repressor, and the like. In the case of a polypeptide, specific binding may involve interactions with polynucleotides, as described above or with molecules or compounds such as agonists, antibodies, antagonists, and the like. Specific binding is dependent upon the presence of structural features that allow appropriate chemical or molecular interactions between molecules.

“Substitution” results from the replacement of one or more nucleotides or amino acids, respectively, by different nucleotides or amino acids. Due to the inherent degeneracy of the genetic code, other polynucleotides that encode functionally equivalent polypeptides may be used to practice the invention.

“Substrate” refers to any rigid or semi-rigid support to which polynucleotides or polypeptides are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

### The Invention

The present invention relates to human and rat polynucleotides that comprise the polynucleotides presented in the Sequence Listing and to the use of these polynucleotides in the diagnosis, study, prevention, and treatment of conditions, diseases, and disorders associated with the presence, absence, or altered expression of the polynucleotide. The polynucleotides have been annotated as electron transfer molecules by their alignment with templates in the LIFESEQ Gold database (Incyte Pharmaceuticals, Palo Alto Calif.) which, in turn, has been annotated with known genes or proteins found in the GenBank or GenPept databases. The polynucleotides claimed herein are regulatory or encoding regions of genes annotated as electron transfer molecules and are described in Tables 1, 2, and 3. The polynucleotides or fragments or complements thereof may be naturally occurring, recombinant, synthetic, or semi-synthetic and are used to identify, isolate or extend identical or related polynucleotides and to identify or purify ligands that specifically bind the polynucleotide from samples or libraries of molecules and compounds. The polynucleotides may also be used in model systems.

The polynucleotides may be altered or joined to a variety of other nucleotide sequences and vectors of interest by means of well-established recombinant DNA techniques. Mutations may be deliberately introduced using techniques such as site-directed mutagenesis, insertion of new linkers, and modification of GC content. In fact, codons may be engineered to mimic the GC content of a particular host with the effect that the rate at which the recombinant vector is expressed in that host is increased. Other reasons for substantially altering the polynucleotide without altering the encoded amino acid sequence include the production of transcripts having more desirable properties, such as a greater half-life, than transcripts produced from a naturally occurring gene.

Vectors of interest include cloning vectors, such as plasmids, cosmids, phage derivatives, phagemids, as well as sequencing, replication, and expression vectors, viruses such as adenoviruses and retroviruses, and the like. In general, such vectors contain an origin of replication functional in at least one organism, convenient restriction endonuclease restriction sites, and selectable markers appropriate for particular host cells. A recombinant vector containing a polynucleotide of the Sequence Listing may be transformed into host cells to produce the desired DNA, RNA, or polypeptide.

The present invention provides for a purified polypeptide or a portion thereof that may be used to screen a library or a plurality of molecules or compounds to identify or purify a ligand that specifically binds the polypeptide. Ligands identified or purified using the polynucleotides or polypeptides of the invention include, but are not limited to, agonists, antagonists, antibodies, carbohydrates, DNA molecules, drug compounds, fatty acids, immunoglobulins, inhibitors, lipids, mimetics, morpholinos, peptide nucleic acids (PNAs), peptides, pharmaceutical agents, proteins, RNA molecules, ribozymes, and the like.

### Derivation of Polynucleotides Encoding Electron Transfer Molecules

mRNA was used to construct cDNA libraries. The cDNA inserts from random isolates of the libraries were sequenced in part, extended, assembled and analyzed as described below. The sequences comprise polynucleotides regulating the expression of or encoding electron transfer molecules as disclosed in the Sequence Listing. These polynucleotides may contain an entire or partial open reading frame or they may encompass splice variants, SNPs, and/or 5′, internal, or 3′ regulatory regions identifiable as or associated with a particular electron transfer molecule.

### Construction of cDNA Libraries

RNA or various samples including bodily fluids, cells, cell lines, or tissues may be purchased from commercial sources such as Clontech Laboratories (Palo Alto Calif.), Stratagene (La Jolla Calif.), the International Institute for Advanced Medicine (Exton Pa.) and the like. Samples may be homogenized and lysed in guanidinium isothiocyanate, in phenol, or in a suitable mixture of denaturants such as TRIZOL reagent (Life Technologies, Gaithersburg Md.). The resulting lysates may be centrifuged over CsCl cushions or extracted with chloroform. RNA may be precipitated with sodium acetate and either isopropanol or ethanol.

Phenol extraction and precipitation of RNA may be repeated as necessary to increase RNA purity. In most cases, the RNA is treated with DNase. Poly(A+) RNA may be isolated using oligo d(T)-coupled paramagnetic particles (Promega, Madison Wis.), OLIGOTEX latex particles or an OLIGOTEX purification kit (Qiagen, Valencia Calif.), or a POLY(A) PURE mRNA purification kit (Ambion, Austin Tex.).

In some cases, companies such as Stratagene will construct custom cDNA libraries with customer provided RNA. Otherwise, cDNA may be synthesized, and cDNA libraries constructed with the UNIZAP vector system (Stratagene) or SUPERSCRIPT plasmid system (Life Technologies), using the recommended procedures of the manufacturer or similar methods known in the art (Ausubel et al. (1997) Short Protocols in Molecular Biology, John Wiley & Sons, New York N.Y., pp. 5.1-6.10; Sambrook et al. (1989) Molecular Cloning, A Laboratory Manual, Cold Spring Harbor Press, Plainview N.Y.). Reverse transcription may be initiated using oligo d(T) or random primers. After synthetic oligonucleotide linkers are ligated to double stranded cDNA, the cDNA may be digested with the appropriate restriction enzyme or enzymes. The cDNA may be size-selected (400-5000 bp) using SEPHACRYL S1000, SEPHAROSE CL2B, or SEPHAROSE CL4B column chromatography (Amersham Pharmacia Biotech, Piscataway N.J.) or preparative agarose gel electrophoresis. cDNAs may be ligated into compatible restriction enzyme sites of the polylinker of one of the following plasmids: PBLUESCRIPT plasmid (Stratagene), pSPORT1 plasmid (Life Technologies), or pINCY plasmid (Incyte Pharmaceuticals). Recombinant plasmids may be transformed into competent Escherichia coli cells including XL1-Blue, XL1-BlueMRF, or SOLR (Stratagene) or DH5α, DH10B, or ElectroMAX DH10B (Life Technologies).

### Isolation of cDNA Clones

Plasmids may be recovered from host cells by in vivo excision using the UNIZAP vector system (Stratagene) or by cell lysis. Plasmids may be purified using one of the following: the Magic or WIZARD Minipreps DNA purification system (Promega); the AGTC MINIPREP purification kit (Edge BioSystems, Gaithersburg Md.); and the REAL PREP 96 plasmid purification kit (Qiagen). Following precipitation, plasmids may be resuspended in 0.1 ml of distilled water and stored, with or without lyophilization, at 4° C.

Alternatively, plasmid DNA may be amplified from host cell lysates using direct link PCR in a high-throughput format (Rao (1994) Anal Biochem 216:1-14). Host cell lysis and thermal cycling steps may be carried out in a single reaction mixture. Samples may be processed and stored in 384-well plates, and the concentration of amplified plasmid DNA may be quantified fluorometrically using PICOGREEN dye (Molecular Probes, Eugene Oreg.) and a FLUOROSKAN II fluorescence scanner (Labsystems Oy, Helsinki Finland).

### Sequencing of the cDNA Clones and Determination of Reading Frame

Methods for DNA sequencing are well known in the art. Conventional enzymatic methods may employ DNA polymerase, Klenow fragment, SEQUENASE, or Taq DNA polymerase (Amersham Pharmacia Biotech) to extend the nucleic acid sequence from an oligonucleotide primer annealed to the DNA template of interest. Methods may use either single-stranded or double-stranded templates. Chain termination reaction products may be electrophoresed on urea-polyacrylamide gels and detected either by autoradiography or by fluorescence. Mechanized reaction preparation, sequencing, and analysis using the fluorescent detection method have permitted expansion in the number of isolates that are prepared and sequenced per day. Machines such as the ABI CATALYST 800 thermal cycler (PE Biosystems, Watertown Mass.) or the DNA ENGINE thermal cycler (MJ Research, Watertown Mass.) may be used in conjunction with the HYDRA microdispenser (Robbins Scientific, Sunnyvale Calif.) or the MICROLAB 2200 liquid transfer system (Hamilton, Reno Nev.) to prepare the cDNAs using reagents provided by Amersham Pharmacia Biotech or supplied in kits such as the ABI PRISM BIGDYE Terminator kit (PE Biosystems). Electrophoretic separation of cDNAs and detection of labeled polynucleotides may be carried out using the MEGABACE 1000 DNA sequencing system (Amersham Pharmacia Biotech) or ABI PRISM 3700, 377 or 373 sequencing systems (PE Biosystems) in conjunction with standard protocols and software known in the art.

Reading frames within the cDNA sequences may be identified using standard methods. It is well known in the art that the DNA of particular organisms (bacteria, plants, and animals) have different GC ratios and tend to display certain triplet periodicities such as a significant preference for pyrimidines in the third codon position. These tendencies have been incorporated into widely available software that may be used to examine coding potential of a given stretch of DNA. Information derived from the algorithm and analysis of start and stop codons may be used to determine proper reading frame with a high degree of certainty.

Even though the polynucleotides of the Sequence Listing have been prepared by current, state-of-the-art, automated methods, they may contain occasional sequencing errors and unidentified nucleotides that are designated by an N. The infrequent sequencing errors or Ns in the polynucleotides of the Sequence Listing do not present a problem to those skilled in the art who wish to practice the invention. Several methods employing standard recombinant techniques, described in Ausubel (supra, pp. 5.1-6.10) may be used to correct errors and complete the missing sequence information. The same cloning and PCR techniques used to obtain a full length sequence may be used to confirm a complete and accurate nucleotide sequence.

### Identification of Sequences and Templates

A clone containing a sequence that is identified by BLAST or BLAST2 (Basic Local Alignment Search Tool: Altschul et al. (1997) Nucleic Acids Res 25:3389-3402; Altschul (1993) J Mol Evol 36:290-300; and Altschul et al. (1990) J Mol Biol 215:403410) or other analyses as encoding an electron transfer molecule may be extended using PCR, subjected to shotgun sequencing, or used as a probe to screen the same library or a mixture of different cDNA libraries commercially available from Life Technologies, or other sources, to identify additional clones. In some cases, a random primed library, or even a genomic library, may be used to obtain the full length sequence. In other cases, a second library may be prepared from the original tissue sample, primed with random primers, and larger inserts identified by screening with the sequence of interest.

PCR is used to extend clones in either direction. U.S. Pat. Nos. 4,683,195 and 4,965,188 describe PCR procedures and enzymes. The oligonucleotides used in such procedures may be designed from the polynucleotides disclosed herein or from adjacent 5′ and 3′ vector sequences. PCR requires two oligomers, that are usually chemically synthesized, to amplify the sequence of interest: one with sense orientation (5′ to 3′), and one with antisense orientation (3′ to 5′). These two oligomers, nested sets of oligomers, or even a degenerate pool of oligomers may be employed under less stringent conditions for identification and/or quantitation of closely related DNA or RNA sequences.

Full length genes may be cloned utilizing restriction-site PCR, inverse PCR, capture PCR, walking PCR, or the extension method presented in U.S. Ser. No. 08/487,112, filed Jun. 7, 1995, that employs the XL-PCR kit (PE Biosystems) and allows the efficient and timely processing of multiple sequences. If a full length cDNA has not been obtained after one passage through an extension procedure, the entire procedure may be repeated using the original library, a library that was size-selected to include only larger cDNAs, or a pool of libraries. The cDNA library may have been prepared by oligo d(T) or random priming; the latter are preferred since they contain more sequences that have 5′ ends of genes. It must be noted that the larger and more complex the polypeptide, the less likely it is that the complete gene will be found in a single plasmid.

Shotgun sequencing may also be used to complete the sequence of a particular cloned insert of interest. Shotgun strategy involves randomly breaking the original insert into segments of various sizes and cloning these fragments into vectors. The fragments are sequenced and reassembled using overlapping ends until the entire sequence of the original insert is known. Shotgun sequencing methods are well known in the art and use thermostable DNA polymerases, heat-labile DNA polymerases, and primers chosen from representative regions flanking the polynucleotides of interest.

A library of plasmids containing inserts of various sizes from about 1 to about 5 kb will provide templates that may be sequenced from both ends to produce sequence that may be assembled using algorithms such as those described below. The size of the DNA insert in new clones may be estimated by agarose gel electrophoresis and the inserts may be sequenced using the techniques and equipment described above or capillary electrophoresis systems (Beckman Coulter, Fullerton Calif.).

Capillary electrophoresis was initially developed to determine the size or nucleotide sequence of PCR products. Such rapid sequencing systems employ flowable polymers for electrophoretic separation, four different fluorescent dyes (one for each nucleotide) for laser activation, and a charge-coupled camera for detection of the emitted wavelengths. Output/light intensity is converted to electrical signal using appropriate software. The entire process from loading of samples to computer analysis and electronic data display is computer controlled. Capillary electrophoresis may potentially provide greater resolution and speed than standard gel based procedures, and it is particularly well suited to sequencing small pieces of DNA that might be present in limited amounts in a particular sample and to identifying SNPs. Early in the development of this technology, Ruiz-Martinez et al. (1993; Anal Chem 65:2851-2858) reported the reproducible sequencing of up to 350 bp of M13 phage DNA in 30 min.

In many instances, the random sequencing of clones from a library will result in the identification of more than one clone containing all or part of a particular gene. If the redundant cDNAs differ in their relative size or in the region of the gene transcribed, it is possible to recombinantly synthesize or chemically combine the various overlapping cDNA fragments to create a single full length cDNA (Caruthers et al. (1980) Nucleic Acids Symp Ser (7) 215-233).

Various programs such as the ABI Assembler and AUTOASSEMBLER software (both PE Biosystems), GCG fragment assembly (Genetics Computer Group, Madison Wis.), CONSED (Gordon (1998) Genome Res 8:195-202), U.S. Pat. No. 5,953,727 and U.S. Ser. No. 09/276,534, filed Mar. 25, 1999, manage sequence assembly projects in a relational database by assembling overlapping sequence fragments into a larger sequence. Generally sequences are placed into assemblages based on relationships between the sequences such as alignment and statistical analysis. Early assembly processes were based on the Meyers Kececioglu model of fragment assembly that uses graph theory as the foundation of a very rigorous multiple sequence alignment program for assembling sequence fragments. Contaminating sequences including vector, mitochondrial, or chimeric sequences may be masked or removed to expedite the assembly of fragments into complete sequences.

### Homology Searches

The polynucleotides of the Sequence Listing may be used to query publically available databases such as GenBank to determine homology to known sequences. Illustrative of computer programs known to those of skill in the art for performing computer-assisted homology searches is BLAST or BLAST2 (Altschul (1997) supra, Altschul (1993) supra, and Altschul (1990) supra). BLAST is especially useful in determining exact matches or in identifying related sequences because of the local nature of the alignments. As described in Karlin and Altschul (1993; Proc Natl Acad Sci 90:5873-5877), the fundamental unit of BLAST algorithm output is the high scoring segment pair (HSP). An HSP consists of two sequence fragments of arbitrary, but equal lengths, whose alignment is locally maximal and for which the alignment score meets or exceeds a threshold or cutoff score set by the user. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the BLAST program output.

BLAST is used with any of the polynucleotides of the present invention to search for HSPs between a query sequence and sequences in a reference nucleotide or protein database. The sequences may be searched against prokaryotic (bacterial) or eukaryotic (animal, fungal or plant) databases and the degree of homology reported. When sequences have sufficiently long regions of agreement or sufficiently high overall agreement, the score of the new cDNA sequence is considered to be an exact match. Allelic sequences, by definition, fit this category in that they differ by about three nucleotides per 100. Exact and homologous matches between the polynucleotides and polypeptides of the present invention and the GenBank databases are shown in Table 3.

Similarly, BLAST may be used to compare the polypeptides of the invention to protein databases such as Swissprot, PFAM, BLOCKS, PRINTS, and the like to identify regions of amino acid homology or common domains. The statistical significance of any polynucleotide or polypeptide matches is evaluated, and those matches that equal or exceed the user-selected threshold of significance are reported. A variety of software and algorithms, that are well known in the art and may be used to analyze the sequences, are described in Ausubel (1997 supra, pp.7.48-7.69), and Meyers (1995; Molecular Biology and Biotechnology, Wiley VCH, New York N.Y., pp. 856-853).

To acquire annotation, the polynucleotides of the Sequence Listing were aligned with assembled templates in the LIFESEQ GOLD database (Incyte Pharmaceuticals). Those templates annotated as electron transfer molecules in the protein functional hierarchy provided the grouping and annotation of the polynucleotides claimed herein as regulatory and coding sequences of a particular electron transfer molecule.

### Use of the Polynucleotide Sequences

The polynucleotides of the invention may be used to express a polypeptide, to identify an identical, similar or related polynucleotide, to screen libraries or a plurality of molecules and compounds and to identify or to purify a ligand. The polynucleotides may also be used for genetic analysis, diagnostic and therapeutic applications for disorders associated with electron transfer molecule expression.

### Expression of the Polynucleotide Sequences

Expression of a particular cDNA may be accomplished by cloning the cDNA into an appropriate vector and transforming this vector into an appropriate host cell. The cloning vector used for the construction of the human and rat cDNA libraries may also be used for expression. Such vectors usually contain a promoter and a polylinker useful for cloning, priming, and transcription. An exemplary vector may also contain the promoter for β-galactosidase, an amino-terminal methionine and the subsequent seven amino acid residues of β-galactosidase. The vector may be transformed into an appropriate host strain of E. coli. Induction of the isolated bacterial strain with isopropylthiogalactoside (IPTG) using standard methods will produce a fusion protein that contains an N terminal methionine, the first seven residues of β-galactosidase, about 15 residues of linker, and the polypeptide encoded by the cDNA.

The cDNA may be shuttled into other vectors known to be useful for expression of protein in specific hosts. Oligonucleotides containing cloning sites and fragments of DNA sufficient to hybridize to stretches at both ends of the cDNA may be chemically synthesized by standard methods. These primers may then be used to amplify the desired fragments by PCR. The fragments may be digested with appropriate restriction enzymes under standard conditions and isolated using gel electrophoresis. Alternatively, similar fragments are produced by digestion of the cDNA with appropriate restriction enzymes and filled in with chemically synthesized oligonucleotides. Fragments of the coding sequence from more than one gene may be ligated together and expressed.

Signal sequences that dictate secretion of soluble proteins are particularly desirable as component parts of a recombinant sequence. For example, a chimeric protein may be expressed that includes one or more additional purification-facilitating domains. Such domains include, but are not limited to, metal-chelating domains that allow purification on immobilized metals, protein A domains that allow purification on immobilized immunoglobulin, and the domain utilized in the FLAGS extension/affinity purification system (Immunex, Seattle Wash.). The inclusion of a cleavable-linker sequence such as ENTEROKINASEMAX (Invitrogen, San Diego Calif.) between the polypeptide and the purification domain may also be used to recover the polypeptide.

Suitable expression hosts may include, but are not limited to, mammalian cells such as Chinese Hamster Ovary (CHO) and human 293 cells, insect cells such as Sf9 cells, yeast cells such as Saccharomyces cerevisiae, and bacteria such as E. coli. For each of these cell systems, a useful expression vector may also include an origin of replication and one or two selectable markers to allow selection in bacteria as well as in a transfected eukaryotic host. Vectors for use in eukaryotic expression hosts may require the addition of 3′ poly(A) tail if the polynucleotide lacks poly(A).

Additionally, the vector may contain promoters or enhancers that increase gene expression. Most promoters are host specific, and they include MMTV, SV40 or metallothionein promoters for CHO cells; trp, lac, tac or T7 promoters for bacterial hosts; or alpha factor, alcohol oxidase or PGH promoters for yeast. Adenoviral vectors with enhancers such as the rous sarcoma virus (RSV) enhancer or retroviral vectors with promoters such as the long terminal repeat (LTR) promoter may be used to drive protein expression in mammalian cell lines. Once homogeneous cultures of recombinant cells are obtained, large quantities of a secreted soluble polypeptide may be recovered from the conditioned medium and analyzed using chromatographic methods well known in the art. An alternative method for the production of large amounts of secreted protein involves the transformation of mammalian embryos and the recovery of the recombinant protein from milk produced by transgenic cows, goats, sheep, and the like.

### Hybridization

The polynucleotides or fragments or complements thereof of the present invention may be used in various hybridization technologies. The polynucleotides may be naturally occurring, recombinant, or chemically synthesized; based on genomic or cDNA sequences; and labeled using a variety of reporter molecules by either PCR or enzymatic techniques. Commercial kits are available for labeling and cleanup of such polynucleotides or probes. Radioactive (Amersham Pharmacia Biotech), fluorescent (Operon Technologies, Alameda Calif.), and chemiluminescent labeling (Promega, Madison Wis.), are well known in the art. Alternatively, a polynucleotide is cloned into a commercially available vector, and probes are produced by transcription. The probe is synthesized and labeled by addition of an appropriate polymerase, such as T7 or SP6 polymerase, and at least one labeled nucleotide.

A probe may be designed or derived from unique regions such as the 3′ untranslated region or from a conserved motif common to electron transfer molecules and used in protocols to identify naturally occurring molecules encoding the mammalian polypeptide, allelic variants, or related molecules. The probe may be DNA or RNA, is usually single stranded and should have at least 50% sequence identity to any of the nucleic acid sequences. The probe may comprise at least 18 contiguous nucleotides of a polynucleotide. Such a probe may be used under hybridization conditions that allow binding only to an identical sequence or under conditions that allow binding to a related sequence with at least one nucleotide substitution. Discovery of related sequences may also be accomplished using a pool of degenerate probes and appropriate hybridization conditions. Generally, a probe for use in Southern or northern hybridizations may be from about 400 to about 4000 nucleotides long. Such probes may be single-stranded or double-stranded and may have high binding specificity in solution-based or substrate-based hybridizations. A probe may also be an oligonucleotide that is used to detect a polynucleotide of the invention in a sample by PCR.

The stringency of hybridization is determined by G+C content of the probe, salt concentration, and temperature. In particular, stringency is increased by reducing the concentration of salt or raising the hybridization temperature. In solutions used for some membrane based hybridizations, addition of an organic solvent such as formamide allows the reaction to occur at a lower temperature. Hybridization may be performed with buffers, such as 5×saline sodium citrate (SSC) with 1% sodium dodecyl sulfate (SDS) at 60° C., that permits the formation of a hybridization complex between nucleic acid sequences that contain some mismatches. Subsequent washes are performed with buffers such as 0.2×SSC with 0.1% SDS at either 45° C. (medium stringency) or 65°-68° C. (high stringency). At high stringency, hybridization complexes will remain stable only where the polynucleotides are completely complementary. In some membrane-based hybridizations, preferably 35% or most preferably 50%, formamide may be added to the hybridization solution to reduce the temperature at which hybridization is performed. Background signals may be reduced by the use of detergents such as Sarkosyl or Triton X-100 (Sigma Aldrich, St. Louis Mo.) and a blocking agent such as denatured salmon sperm DNA. Selection of components and conditions for hybridization are well known to those skilled in the art and are reviewed in Ausubel (supra, pp. 6.11-6.19, 14.11-14.36, and A1-43).

Dot-blot, slot-blot, low density and high density arrays are prepared and analyzed using methods known in the art. Probes or array elements from about 18 consecutive nucleotides to about 5000 consecutive nucleotides are contemplated by the invention and used in array technologies. The preferred number of probes or array elements is at least about 40,000; a more preferred number is at least about 10,000, and a most preferred number is at least about 600 to about 800. The array may be used to monitor the expression level of large numbers of genes simultaneously and to identify genetic variants, mutations, and SNPs. Such information may be used to determine gene function; to understand the genetic basis of a disorder; to diagnose a disorder; and to develop and monitor the activities of therapeutic agents being used to control or cure a disorder. (See, e.g., U.S. Pat. No. 5,474,796; PCT application WO95/11995; PCT application WO95/35505; U.S. Pat. Nos. 5,605,662; and 5,958,342.)

### Diagnostic Uses of the Polynucleotide

The polynucleotides of the Sequence Listing or their fragments or complements may be used as probes, individually or as a plurality of compositions, in diagnostic assays for the detection of an identical or related polynucleotide. A probe for the detection of related sequence is selected from a region of the polynucleotide encoding a “conserved” domain, motif, or region of interest. For the detection of identical sequences, or where maximum specificity is desired, probes are selected from a unique region of the polynucleotide such as the 3′ untranslated region. Such probes may be pretested for their ability to identify or amplify a target sequence. Optimization of the probe in the protocol, as commonly practiced by those of skill in the art, should reduce the frequency of false positives and false negatives.

Diagnostic assays known to those of skill in the art may be used to detect disorders associated with altered levels of expression. A probe developed from a polynucleotide encoding an electron transfer molecule is labeled with a reporter molecule and added to a sample from a subject under amplifying or hybridizing conditions. After a time sufficient for binding to occur, the signal from the reporter molecule is quantitated and compared with standards derived from samples taken from normal subjects or those diagnosed with the disorder. If polynucleotide expression varies significantly from the normal standard and approximates the level of expression correlated with the presence of the disorder, the assay indicates the presence of the disorder. Such qualitative or quantitative methods for diagnosing a disorder may include Southern, northern, dot blot, or other membrane or dip-stick based technologies or multiple-sample format technologies such as PCR, ELISA-like, pin, or array-based assays.

Such assays, combining a subject sample with one or more probes, are also applicable in evaluating the efficacy of a particular therapeutic treatment regime. They may also be used in animal studies, preclinical tests, clinical trials, or monitoring the treatment of an individual subject. Standards for use in diagnostic assay are developed as follows: First, the normal standard is developed by processing a statistically significant number of normal samples and evaluating the range and variation of expression of the polynucleotide definitive for those samples. Second, samples from animals or subjects with the disorder are also evaluated to establish range and variation of expression of the polynucleotide. Third, the differences between the normal and diseased samples are compared and deviation is evaluated for statistical significance. If these standards are acceptable and reliable, then samples from subjects who are being treated with an existing therapeutic agent may be compared with the standards to generate a treatment profile. The assay is repeated to determine whether the profile progresses toward or returns to the normal standard or pattern of gene expression. Successive treatment profiles may be used to show the efficacy of treatment over a period of several days or several months.

Similar assays may be used in animal studies testing potential pharmacological agents. Successive treatment profiles may be used to show the efficacy and toxicity of the agent over a period of several days or several months or in the study of disease progression in model systems described infra.

### Transcript Imaging

Another aspect of the present invention relates to diagnostic or treatment methods based on specific imaging of the polynucleotides of the present invention. As used herein, the profile of polynucleotides that reflect gene transcription activity in a particular tissue at a particular time, is defined as a “transcript image”. Such profiles are generated by naming, matching, and counting all copies of related clones and arranging them in order of abundance. The process of producing a comparative transcript image is fully described in U.S. Pat. No. 5,840,484.

Subtractions between transcript images of different cells or tissues or of the same cells or tissues under different conditions may be used to discern differences in cellular activities. For example, a transcript image could show differences occurring between two different tissues, such as liver and brain tissues; between normal and diseased tissue, such as normal and cancerous brain tissue; or between untreated and treated tissues, such as brain tissue with astrocytoma before and after chemotherapy or radiation treatment.

Larger numbers of mRNA transcripts, as represented by their respective cDNA clones, may be compared using computer-based or “electronic subtraction” methods rather than using analogous laboratory methods, such as northern blot analysis. Electronic subtraction between any two transcript images parallels hybrid subtraction between any two cDNA libraries using techniques that are known to those of skill in the art (Meyers, supra, pp. 698-699).

The entire set or a selected subset of tissue-specific, unique, or homologous genes may be used in hybridization technologies for diagnosis of inherited or acquired conditions. Such markers have the potential to improve diagnosis (even in pre-symptomatic or prenatal conditions), identify appropriate therapeutic molecules, and assess treatment efficacy by monitoring changes in expression of polynucleotides during treatment.

### Genetic Analysis

Gene identification and mapping are important in the investigation and treatment of mammalian disorders. Cancer, arthritis, diabetes, and the like are of interest. Each of these disorders is more complex than the single gene defects of sickle cell anemia or cystic fibrosis and involve genes of predictive and therapeutic value. Mapping of these genes is a complex and reiterative process and generally proceeds from genetic linkage analysis to physical mapping. Individual genetic markers and their known locations often serve to indicate which regions of chromosomes may contain genes coexpressed in the disorders or sequences that could be used in familial investigations for genetic lesions or SNPs attributable to that disorder. Examples of genetic linkage maps are found in various scientific journals or at the Online Mendelian Inheritance in Man (OMIM) world wide web site. Similarly genes mapped to a chromosome that shows an inversion, translocation, or the like associated with a particular disorder may help elucidate the etiology of that disorder.

The polynucleotides or probes of the invention are useful in mapping naturally occurring genomic sequences. The sequence may be mapped to a particular chromosome or to a specific region of the chromosome using well known techniques. These include hybridization to chromosomal spreads (Verma et al. (1988) Human Chromosomes: A Manual of Basic Techniques, Pergamon Press, New York N.Y.), flow-sorted chromosomal preparations, or cDNA libraries made from artificial chromosome constructions such as yeast artificial chromosomes (YACs), bacterial artificial chromosomes (BACs), bacterial P1 constructions, or single chromosomes.

Hybridization of chromosomal preparations of another mammalian species, such as mouse, may reveal associated markers even if the number or arm of the corresponding human or rat chromosome is not known. These new marker sequences are mapped to human chromosomes and provide valuable information to investigators searching for disease genes using positional cloning or other gene discovery techniques. Once a disease or syndrome has been crudely correlated by genetic linkage with a particular genomic region, e.g., ataxia-telangiectasia to 11q22-23, any sequences mapping to that area may represent associated or regulatory genes for further investigation (Gatti et al. (1988) Nature 336:577-580). The polynucleotides of the invention may also be used to detect differences in chromosomal architecture due to translocation, inversion, etc., among normal, carrier, or affected individuals and are potentially useful in legal or forensic studies to determine paternity or to establish the presence, absence, or activities of an accused suspect in a particular crime.

A physical map is necessary for determining the order of marker genes in a particular chromosomal region. Physical mapping techniques are well known in the art and require the generation of overlapping sets of cloned DNA fragments, both expressed sequence tags and sequence tag sites, from a particular artificial chromosome construction, organelle, chromosome, or genome. These clones are analyzed to reconstruct and catalog their order. Once the position of a marker is determined, the DNA from that region is obtained by consulting the catalog and selecting clones from that region. The gene of interest is located through positional cloning techniques using hybridization or similar methods.

### Therapeutic Uses of the Polynucleotide

A polynucleotide may be useful in the treatment of various disorders associated with its altered expression. By introducing polynucleotides into cells, gene therapy may be used to treat disorders in which there is an absence of expression (due to a genetic mutation) or underexpression. For example, delivery of a cDNA or mRNA encoding the cystic fibrosis transmembrane regulator into the lungs of a cystic fibrosis patient may provide transient expression and relief from the symptoms of the disease (Harvey et al. (1999) J Clin Invest 104:1245-1255). Alternatively, disorders associated with an increase in expression of the polynucleotide may also be treated by the introduction of a sense or antisense molecule designed to inhibit the transcription or translation of that specific polynucleotide. Such RNA and DNA molecules and ribozymes that function to inhibit transcription or translation of a native gene are within the scope of the invention. For example, molecules that bind directly to the targeted polynucleotide, between −10 and +10 nucleotides of the coding region start site, will inhibit transcription.

Similarly, it is contemplated that ribozymes may be designed to catalyze the specific cleavage of an overexpressed mRNA. The mechanism of ribozyme activity involves sequence-specific hybridization of the ribozyme molecule to its complementary target RNA followed by an endonucleolytic cleavage. Specific ribozyme cleavage sites within any potential RNA target are initially identified by scanning the target molecule for ribozyme cleavage sites that include the following triplet nucleotides: GUA, GUU and GUC.

Sense molecules, antisense molecules and ribozymes of the invention may be prepared by any method known in the art for the synthesis of RNA molecules, including solid phase phosphoramidite chemical synthesis or recombinant in vitro transcription. Expression vectors derived from viruses (such as retroviruses, vaccinia, adenoviruses, herpes, or bovine papilloma viruses) may be used to deliver recombinant polynucleotides into the targeted cell population. Methods that are well known to those of skill in the art and described in Ausubel (supra, pp. 9.1-9.57) may be used to construct recombinant viral vectors containing a polynucleotide of the present invention. Alternately, recombinant polynucleotides, or their full length cDNAs, may be encapsulated in liposomes, frozen into ice microcrystals, precipitated on metal (gold) microparticles, and the like for delivery via electroporation, particle bombardment, microinjection, transfection, and the like into target cells. Transformation techniques will vary according to whether ex vivo or in vivo gene therapy is being attempted.

Knowledge of the correct, complete cDNA sequence will enable the use of antisense technology in the investigation of new gene functions. In this method, either oligonucleotides or cDNA fragments representing the sense or antisense molecule may be used either in vitro or in vivo to inhibit expression. After transformation with one of these molecules, a gene of interest may be effectively masked or turned off. Frequently, additional functions of that polynucleotide or the encoded polypeptide can be ascertained by observation of cellular or organismic behavior such as changes in surface antigens or changes in mobility or morphology.

### Use of the Polypeptides Encoded by the Polynucleotides

The polynucleotides of this application or their full length cDNAs may be used to produce purified polypeptides using recombinant DNA technologies described herein and taught in Ausubel (sura; pp. 16.1-16.62). One of the advantages of producing polypeptides by these procedures is the ability to obtain highly-enriched sources of the polypeptides thereby simplifying purification procedures.

The present invention also encompasses amino acid substitutions, deletions or insertions made on the basis of similarity in polarity, charge, solubility, hydrophobicity, hydrophilicity, and/or the amphipathic nature of the residues involved. Such substitutions may be conservative in nature when the substituted residue has structural or chemical properties similar to the original residue (e.g., replacement of leucine with isoleucine or valine) or they may be nonconservative when the replacement residue is radically different (e.g., a glycine replaced by a tryptophan). Computer programs included in LASERGENE software (DNASTAR, Madison Wis.), MACVECTOR software (Genetics Computer Group, Madison Wis.) and RasMol software (www.umass.edu/microbio/rasmol) may be used to help determine which and how many amino acid residues in a particular portion of the polypeptide may be substituted, inserted, or deleted without abolishing biological or immunological activity.

In addition to recombinant production, polypeptides or portions thereof may be produced using solid-phase techniques (Stewart et al. (1969) Solid-Phase Peptide Synthesis, W H Freeman, San Francisco Calif.; Merrifield (1963) J Am Chem Soc 5:2149-2154), manually, or using machines such as the ABI431A Peptide synthesizer (PE Biosystems). Polypeptides produced by any of the above methods may be used as pharmaceutical compositions to treat disorders associated with underexpression.

### Production of Antibodies

A polypeptide encoded by a polynucleotide of the invention may be used to produce specific antibodies. Antibodies may be produced using an oligopeptide or a portion of the polypeptide with inherent immunological activity. Methods for producing antibodies include: 1) injecting an animal (usually goats, rabbits, or mice) with the polypeptide, or a portion or an oligopeptide thereof, to induce an immune response; 2) engineering hybridomas to produce monoclonal antibodies; 3) inducing in vivo production in the lymphocyte population; or 4) screening libraries of recombinant immunoglobulins. Recombinant immunoglobulins may be produced as taught in U.S. Pat. No. 4,816,567.

Antibodies produced using the polypeptides of the invention are useful for the diagnosis of prepathologic disorders as well as the diagnosis of chronic or acute diseases characterized by abnormalities in the expression, amount, or distribution of the polypeptide. A variety of protocols for competitive binding or immunoradiometric assays using either polyclonal or monoclonal antibodies specific for polypeptides are well known in the art. Immunoassays typically involve the formation of complexes between a polypeptide and its specific binding molecule or compound and the measurement of complex formation. A two-site, monoclonal-based immunoassay utilizing monoclonal antibodies reactive to two noninterfering epitopes on a specific polypeptide is preferred, but a competitive binding assay may also be employed.

Immunoassay procedures may be used to quantify expression of the polypeptide in cell cultures, in subjects with a particular disorder or in model animal systems under various conditions. Increased or decreased production of polypeptides as monitored by immunoassay may contribute to knowledge of the cellular activities associated with developmental pathways, engineered conditions or diseases, or treatment efficacy. The quantity of a given polypeptide in a given tissue may be determined by performing immunoassays on freeze-thawed detergent extracts of biological samples and comparing the slope of the binding curves to binding curves generated by purified polypeptide.

The polypeptides and antibodies may be labeled for purposes of assay by joining them, either covalently or noncovalently, with a reporter molecule that provides for a detectable signal. A wide variety of labels and conjugation techniques are known and have been reported in the scientific and patent literature including, but not limited to U.S. Pat. Nos. 3,817,837; 3,850,752; 3,939,350; 3,996,345; 4,277,437; 4,275,149; and 4,366,241.

### Therapeutic Uses of the Polypeptide or an Antibody Produced against the Polypeptide

The polypeptide encoded by the polynucleotide of the invention may be used therapeutically in subjects diagnosed as lacking sufficient biologically active polypeptide. These subjects may lack a functional gene, may not be able to express the polypeptide, or may underexpress the polypeptide. In such cases, the polypeptide may be supplied in a pharmaceutical composition. Conversely, an antibody produced using the polypeptide or a portion thereof that specifically binds the polypeptide may be administered as a pharmaceutical composition to a subject in which overexpression of the polynucleotide or its encoded polypeptide are contributing to a disorder.

If the polypeptide is particularly hard to produce, stabilize or formulate as a pharmaceutical composition, rational drug design may be used to produce structural analogs with similar biological activity. In one approach, the three-dimensional structure of a polypeptide, or of a polypeptide-inhibitor complex, is determined by X-ray crystallography, by computer modeling or, most typically, by a combination of the two approaches. Both the shape and charges of the polypeptide must be ascertained to elucidate the structure and to determine active site(s) of the molecule. Less often, useful information regarding the structure of a polypeptide may be gained by modeling based on the structure of homologous proteins. In both cases, relevant structural information is used to design analogous molecules or to identify efficient inhibitors.

In another approach, ligands that are highly specific for the polypeptide may be used as a pharmacore to screen for small molecules that can be tested for drug efficacy. Drug libraries often contain small peptides or mimetics that are more active or stable than the original polypeptide and may be used in its stead.

It is also possible to use the polypeptide to isolate a target-specific antibody that can be used as a pharmacore upon which subsequent drug design can be based. By generating anti-idiotypic antibodies (anti-ids) to a functional, pharmacologically active antibody, the binding site of the anti-id is an analog of the original receptor. The anti-id can then be used to identify and isolate peptides or mimetics from libraries of chemically or biologically produced molecules. The isolated peptide can then be tested for inhibition, toxicity, pharmaceutical efficacy and, if appropriate, formulated as a pharmaceutical composition.

### Screening for Useful Compounds

Both the polynucleotides and polypeptides are particularly useful for screening libraries of molecules or test compounds for identification of ligands that bind specifically to them. For example, a polynucleotide or fragment thereof may be combined with a plurality of natural, synthetic, or inorganic molecules to screen for ligands that may function as transcription factors, enhancers, repressors, and the like that modulate gene expression. Similarly, a polypeptide or a portion thereof may be combined with a library or a plurality of molecules or compounds to screen for ligands that specifically bind to them and may function as agonists or antagonists.

### Screening and Purification Assays

The polynucleotide encoding the mammalian polypeptide may be used to screen a library or a plurality of molecules or compounds for a ligand with specific binding affinity. The ligands may be DNA molecules, RNA molecules, PNAs, peptides, proteins such as transcription factors, enhancers, repressors, and other proteins that regulate the activity, replication, transcription, or translation of the polynucleotide in the biological system. The assay involves combining the mammalian polynucleotide or a fragment thereof with the molecules or compounds under conditions that allow specific binding and detecting the bound polynucleotide to identify at least one ligand that specifically binds the polynucleotide.

In one embodiment, the polynucleotide of the invention may be incubated with a library of isolated and purified molecules or compounds and binding activity determined by methods well known in the art, e.g., a gel-retardation assay (U.S. Pat. No. 6,010,849) or a reticulocyte lysate transcriptional assay. In another embodiment, the polynucleotide may be incubated with nuclear extracts from biopsied and/or cultured cells and tissues. Specific binding between the polynucleotide and a molecule or compound in the nuclear extract is initially determined by gel shift assay and may be later confirmed by raising antibodies against that molecule or compound. When these antibodies are added into the assay, they cause a supershift in the gel-retardation assay.

In another embodiment, the polynucleotide may be used to purify a molecule or compound using affinity chromatography methods well known in the art. In one embodiment, the polynucleotide is chemically reacted with cyanogen bromide groups on a polymeric resin or gel. Then a sample is passed over and reacts with or binds to the polynucleotide. The molecule or compound which is bound to the polynucleotide may be released from the polynucleotide by increasing the salt concentration of the flow-through medium and collected.

Similarly the polypeptide or a portion thereof encoded by the polynucleotide may be used to screen libraries or a plurality of molecules or compounds for a ligand with specific binding affinity or to purify a molecule or compound from a sample. The polypeptide or portion thereof employed in such screening may be free in solution, affixed to an abiotic or biotic substrate, or located intracellularly. For example, viable or fixed prokaryotic host cells that are stably transformed with recombinant nucleic acids that have expressed and positioned a polypeptide on their cell surface can be used in screening assays. The cells are screened against libraries or a plurality of ligands and the specificity of binding or formation of complexes between the expressed polypeptide and the ligand may be measured. The ligands may be DNA, RNA, or PNA molecules, agonists, antagonists, antibodies, immunoglobulins, inhibitors, peptides, pharmaceutical agents, proteins, drugs, or any other test molecule or compound that specifically binds the polypeptide. An exemplary assay involves combining the mammalian polypeptide or a portion thereof with the molecules or compounds under conditions that allow specific binding and detecting the bound polypeptide to identify at least one ligand that specifically binds the polypeptide.

This invention also contemplates the use of competitive drug screening assays in which neutralizing antibodies capable of binding the polypeptide specifically compete with a test compound capable of binding to the polypeptide or oligopeptide or fragment thereof. One method for high throughput screening using very small assay volumes and very small amounts of test compound is described in U.S. Pat. No. 5,876,946. Molecules or compounds identified by screening may be used in a mammalian model system to evaluate their toxicity, diagnostic, or therapeutic potential.

### Labeling of Molecules for Use in Arrays or Assays

A wide variety of reporter molecules and conjugation techniques are known by those skilled in the art and may be used in various polynucleotide, polypeptide or antibody arrays or assays. Synthesis of labeled molecules may be achieved using Promega or Amersham Pharmacia Biotech kits for incorporation of a labeled nucleotide such as ³²P-dCTP, Cy3-dCTP or Cy5-dCTP or amino acid such as ³⁵S-methionine. Polynucleotides, polypeptides, or antibodies may be directly labeled with a reporter molecule by chemical conjugation to amines, thiols and other groups present in the molecules using reagents such as BIODIPY or FITC (Molecular Probes, Eugene Oreg.).

### Purification of a Ligand

The polynucleotide or a fragment thereof may be used to purify a ligand from a sample. A method for using a mammalian polynucleotide or a fragment thereof to purify a ligand would involve combining the polynucleotide or a fragment thereof with a sample under conditions to allow specific binding, recovering the bound polynucleotide, and using an appropriate agent to separate the polynucleotide from the purified ligand.

Similarly, the polypeptide or a portion thereof may be used to purify a ligand from a sample. A method for using a mammalian polypeptide or a portion thereof to purify a ligand would involve combining the polypeptide or a portion thereof with a sample under conditions to allow specific binding, recovering the bound polypeptide, and using an appropriate chaotropic agent to separate the polypeptide from the purified ligand.

### Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of underexpression or overexpression of genes of interest and for the development of methods for diagnosis and treatment of diseases. A mammal inbred to overexpress a particular gene (for example, secreted in milk) may also serve as a convenient source of the protein expressed by that gene.

### Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess potential consequences on human health following exposure to the agent.

Genetic toxicology identifies and analyzes the effect of an agent on the rate of endogenous, spontaneous, and induced genetic mutations. Genotoxic agents usually have common chemical or physical properties that facilitate interaction with nucleic acids and are most harmful when chromosomal aberrations are transmitted to progeny. Toxicological studies may identify agents that increase the frequency of structural or functional abnormalities in the tissues of the progeny if administered to either parent before conception, to the mother during pregnancy, or to the developing organism. Mice and rats are most frequently used in these tests because their short reproductive cycle allows the production of the numbers of organisms needed to satisfy statistical requirements.

Acute toxicity tests are based on a single administration of an agent to the subject to determine the symptomology or lethality of the agent. Three experiments are conducted: 1) an initial dose-range-finding experiment, 2) an experiment to narrow the range of effective doses, and 3) a final experiment for establishing the dose-response curve.

Subchronic toxicity tests are based on the repeated administration of an agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, with a duration of a year or more, are used to demonstrate either the absence of toxicity or the carcinogenic potential of an agent. When studies are conducted on rats, a minimum of three test groups plus one control group are used, and animals are examined and monitored at the outset and at intervals throughout the experiment.

### Transgenic Animal Models

Transgenic rodents that overexpress or underexpress a gene of interest may be inbred and used to model human diseases or to test therapeutic or toxic agents. (See, e.g., U.S. Pat. Nos. 5,175,383 and 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

### Embryonic Stem Cells

Embryonic (ES) stem cells isolated from rodent embryos retain the potential to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gene, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types that differentiate into, for example, neural cells, hematopoietic lineages, and cardiomyocytes.

### Knockout Analysis

In gene knockout analysis, a region of a mammalian gene is enzymatically modified to include a non-mammalian gene such as the neomycin phosphotransferase gene (neo; Capecchi (1989) Science 244:1288-1292). The modified gene is transformed into cultured ES cells and integrates into the endogenous geneome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines that lack a functional copy of the mammalian gene. In one example, the mammalian gene is a human gene.

### Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with potential pharmaceutical agents to obtain information on treatment of the analogous human condition. These methods have been used to model several human diseases.

As described herein, the uses of the polynucleotides, provided in the Sequence Listing of this application, and their encoded polypeptides are exemplary of known techniques and are not intended to reflect any limitation on their use in any technique that would be known to the person of average skill in the art. Furthermore, the polynucleotides provided in this application may be used in molecular biology techniques that have not yet been developed, provided the new techniques rely on properties of nucleotide sequences that are currently known to the person of ordinary skill in the art, e.g., the triplet genetic code, specific base pair interactions, and the like. Likewise, reference to a method may include combining more than one method for obtaining or assembling full length cDNA sequences that will be known to those skilled in the art.

It is to be understood that the invention is not to be limited only to these particular sequences, variants, formulations or methods. The terminology and definitions are not intended to be limiting since the scope of protection will ultimately depend upon the claims. In like manner, the examples below are provided for the purpose of illustrating, rather than limiting, the subject invention.

### EXAMPLES

### Isolation, Sequence Analysis and Use of Electron Transfer Molecules

I cDNA Library Construction

The BRAIFET01 cDNA library was constructed from brain tissue derived from a 23-week-old Caucasian male fetus (specimen #RB96-04-0157-014) who was stillborn. Serology was negative. Patient history included a hypoplastic heart.

The frozen tissue was homogenized and lysed in TRIZOL reagent (1 g tissue/10 ml TRIZOL; Life Technologies) using a POLYTRON homogenizer (PT-3000; Brinkmann Instruments, Westbury N.Y.). After a brief incubation on ice, chloroform was added (1:5 v/v), and the lysate was centrifuged. The chloroform layer was discarded, and the RNA precipitated with isopropanol, resuspended in DEPC-treated water, and treated with DNase for 25 min at 37 ° C. The mRNA was re-extracted twice with acid phenol-chloroform, pH 4.7, and precipitated using 0.3M sodium acetate and 2.5 volumes ethanol. The mRNA was isolated with the OLIGOTEX kit (Qiagen) and used to construct the cDNA library.

The mRNA was handled according to the recommended protocols in the SUPERSCRIPT plasmid system (Life Technologies). The cDNAs were fractionated on a SEPHAROSE CL4B column (Amersham Pharmacia Biotech), and those cDNAs exceeding 400 bp were ligated into pINCY 1 plasmid. The plasmid was subsequently transformed into DH5α competent cells (Life Technologies).

II Construction of pINCY Plasmid

The plasmid was constructed by digesting the pSPORT1 plasmid (Life Technologies) with EcoRI restriction enzyme (New England Biolabs, Beverly Mass.) and filling the overhanging ends using Klenow enzyme (New England Biolabs) and 2′-deoxynucleotide 5′-triphosphates (dNTPs). The plasmid was self-ligated and transformed into the bacterial host, E. coli strain JM109.

An intermediate plasmid produced by the bacteria (pSPORT 1-ΔRI) showed no digestion with EcoRI and was digested with Hind III (New England Biolabs) and the overhanging ends were again filled in with Klenow and dNTPs. A linker sequence was phosphorylated, ligated onto the 5′ blunt end, digested with EcoRI, and self-ligated. Following transformation into JM109 host cells, plasmids were isolated and tested for preferential digestibility with EcoRI, but not with Hind III. A single colony that met this criteria was designated pINCY 1 plasmid.

After testing the plasmid for its ability to incorporate cDNAs from a library prepared using NotI and EcoRI restriction enzymes, several clones were sequenced; and a single clone containing an insert of approximately 0.8 kb was selected from which to prepare a large quantity of the plasmid. After digestion with NotI and EcoRI, the plasmid was isolated on an agarose gel and purified using a QIAQUICK column (Qiagen) for use in library construction.

III Normalization of cDNA Libraries

For purposes of example, the normalization of a human brain library is described. About 4.9×10⁶ independent clones of the BRAINON01 plasmid library in E. coli strain DH12S competent cells (Life Technologies) were grown in liquid culture under carbenicillin (25 mg/l) and methicillin (1 mg/ml) selection following transformation by electroporation. To reduce the number of excess cDNA copies according to their abundance levels in the library, the cDNA library was then normalized in a single round according to the procedure of Soares et al. (1994, Proc Natl Acad Sci 91:9228-9232), with the following modifications. The primer to template ratio in the primer extension reaction was increased from 2:1 to 10:1. The ddNTP concentration in the reaction was reduced to 150 μM for each ddNTP to allow the generation of longer (400-1000 nt) primer extension products. The reannealing hybridization was extended from 13 to 48 hr. The single stranded DNA circles of the normalized library were purified by hydroxyapatite chromatography and converted to partially double-stranded by random priming, followed by electroporation into E. coli strain DH10B competent cells (Life Technologies).

IV Isolation and Sequencing of cDNA Clones

Plasmid DNA was released from the cells and purified using the REAL PREP 96 plasmid kit (Qiagen). This kit enabled the simultaneous purification of 96 samples in a 96-well block using multi-channel reagent dispensers. The recommended protocol was employed except for the following changes: 1) the bacteria were cultured in 1 ml of sterile Terrific Broth (Life Technologies) with carbenicillin at 25 mg/l and glycerol at 0.4%; 2) after inoculation, the cultures were incubated for 19 hr and at the end of incubation, the cells were lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4° C.

The cDNAs were prepared using a MICROLAB 2200 (Hamilton) in combination with DNA ENGINE thermal cyclers (MJ Research). The cDNAs were sequenced by the method of Sanger and Coulson (1975; J Mol Biol 94:441-448) using ABI PRISM 377 and 373 DNA sequencing systems (PE Biosystems) and standard ABI protocols and kits. The solution volumes were used at 0.25×-1.0×concentrations. Some of the sequences disclosed herein were sequenced using different solutions and dyes which, unless otherwise noted, came from Amersham Pharmacia Biotech.

V. Extension of cDNA Sequences

The polynucleotides were extended using a cDNA clone and oligonucleotide primers. One primer was synthesized to initiate 5′ extension of the known fragment, and the other, to initiate 3′ extension of the known fragment. The initial primers were designed using OLIGO 4.06 software (National Biosciences, Plymouth Minn.), or another appropriate program, to be about 22 to 30 nucleotides in length, to have a GC content of about 50% or more, and to anneal to the target sequence at temperatures of about 68° C. to about 72° C. Any stretch of nucleotides that would result in hairpin structures and primer-primer dimerizations was avoided.

Selected cDNA libraries were used as templates to extend the sequence. If more than one extension was necessary or desired, additional or nested sets of primers were designed. Preferred libraries are ones that have been size-selected to include larger cDNAs. Also, random primed libraries are preferred because they will contain more sequences with the 5′ and upstream regions of genes. A randomly primed library is particularly useful if an oligo d(T) library does not yield a full length cDNA. Genomic libraries are useful for extension 5′ of the promoter binding region in order to obtain regulatory elements.

High fidelity amplification was obtained by PCR using methods such as that taught in U.S. Pat. No. 5,932,451. PCR was performed in 96-well plates using the DNA ENGINE thermal cycler (MJ Research). The reaction mix contained DNA template, 200 nmol of each primer, reaction buffer containing Mg²⁺, (NH₄)₂SO₄, and β-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B (Incyte Pharmaceuticals): Step 1: 94° C., three min; Step 2: 94° C., 15 sec; Step 3: 60° C., one min; Step 4: 68° C., two min; Step 5: Steps 2, 3, and 4 repeated 20 times; Step 6: 68° C., five min; Step 7: storage at 4° C. In the alternative, the parameters for primer pair T7 and SK+ (Stratagene) were as follows: Step 1: 94° C., three min; Step 2: 94° C., 15 sec; Step 3: 57° C., one min; Step 4: 68° C., two min; Step 5: Steps 2, 3, and 4 repeated 20 times; Step 6: 68° C., five min; Step 7: storage at 4° C.

The concentration of DNA in each well was determined by dispensing 100 μl PICOGREEN quantitation reagent (0.25% reagent in 1×TE, v/v; Molecular Probes) and 0.5 μl of undiluted PCR product into each well of an opaque fluorimeter plate (Corning, Acton Mass.) and allowing the DNA to bind to the reagent. The plate was scanned in a Fluoroskan II (Labsystems Oy) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleotide sequences were desalted, concentrated, transferred to 384-well plates, digested with CviJI cholera virus endonuclease (Molecular Biology Research, Madison Wis.), and sonicated or sheared prior to religation into pUC18 vector (Amersham Pharmacia Biotech). For shotgun sequences, the digested nucleotide sequences were separated on low concentration (0.6 to 0.8%) agarose gels, fragments were excised, and the agar was digested with AGARACE enzyme (Promega). Extended clones were religated using T4 DNA ligase (New England Biolabs) into pUC 18 vector (Amersham Pharmacia Biotech), treated with Pfu DNA polymerase (Stratagene) to fill-in restriction site overhangs, and transfected into E. coli competent cells. Transformed cells were selected on antibiotic-containing media, and individual colonies were picked and cultured overnight at 37° C. in 384-well plates in LB/2×carbenicillin liquid media.

The cells were lysed, and DNA was amplified using primers, Taq DNA polymerase (Amersham Pharmacia Biotech) and Pfu DNA polymerase (Stratagene) with the following parameters: Step 1: 94° C., three min; Step 2: 94° C., 15 sec; Step 3: 60° C., one min; Step 4: 72° C., two min; Step 5: steps 2, 3, and 4 repeated 29 times; Step 6: 72° C., five min; Step 7: storage at 4° C. DNA was quantified using PICOGREEN quantitative reagent (Molecular Probes) as described above. Samples with low DNA recoveries were reamplified using the conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (Amersham Pharmacia Biotech) or the ABI PRISM BIGDYE terminator cycle sequencing kit (PE Biosystems).

VI Homology Searching of cDNA Clones and Their Deduced Polypeptides

The polynucleotides of the Sequence Listing or their deduced amino acid sequences were used to query databases such as GenBank, SwissProt, BLOCKS, and the like. These databases that contain previously identified and annotated sequences or domains were searched using BLAST or BLAST 2 (Altschul (1997) supra, Altschul (1993) supra, and Altschul (1990) supra) to produce alignments and to determine which sequences were exact matches or homologs. The alignments were to sequences of prokaryotic (bacterial) or eukaryotic (animal, fungal, or plant) origin. Alternatively, algorithms such as the one described in Smith and Smith (1992, Protein Engineering 5:35-51) could have been used to deal with primary sequence patterns and secondary structure gap penalties. All of the sequences disclosed in this application have lengths of at least 49 nucleotides, and no more than 12% uncalled bases (where N is recorded rather than A, C, G, or T).

As detailed in Karlin (supra), BLAST matches between a query sequence and a database sequence were evaluated statistically and only reported when they satisfied the threshold of 10⁻²⁵ for nucleotides and 10⁻¹⁴ for peptides. The expressed sequence tags (ESTs) comprising the mammalian polynucleotides regulating or encoding electron transfer molecules were searched against the GenBank databases for homology. Homology of human ESTs was evaluated by product score calculated as follows: the % nucleotide or amino acid identity [between the query and reference sequences] in BLAST is multiplied by the % maximum possible BLAST score [based on the lengths of query and reference sequences] and then divided by 100. In the alternative, the product score for human and rat sequences was calculated as follows: the BLAST score is multiplied by the % nucleotide identity and the product is divided by (5 times the length of the shorter of the two sequences), such that a 100% alignment over the length of the shorter sequence gives a product score of 100. In comparison with hybridization procedures used in the laboratory, the electronic stringency for an exact match was set at 70, and the conservative lower limit for an exact match was set at approximately 40 (with 1-2% error due to uncalled bases).

The BLAST software suite, freely available sequence comparison algorithms (NCBI, Bethesda Md.; http://www.ncbi.nlm.nih.gov/gorf/bl2.html) includes various sequence analysis programs including “blastn”, that is used to align a known nucleic acid molecules, BLAST 2 that is used for direct pairwise comparison of either nucleic or amino acid molecules. BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap x drop-off: 50; Expect: 10; Word Size: 11; and Filter: on. Identity or similarity may be measured over the entire length of a sequence or some smaller portion thereof. Brenner et al. (1998; Proc Natl Acad Sci 95:6073-6078, incorporated herein by reference) analyzed the BLAST for its ability to identify structural homologs by sequence identity and found 30% identity is a reliable threshold for sequence alignments of at least 150 residues and 40%, for alignments of at least 70 residues.

The mammalian polynucleotides of this application were compared with assembled consensus sequences or templates found in the LIFESEQ GOLD database. Component sequences from cDNA, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns”, or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another and those having local similarity of at least 82% were combined and reassembled. Bins having templates with less than 95% local identity were split. Templates were subjected to analysis by STITCHER/EXON MAPPER algorithms that analyze the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of ≦1×10⁻⁸. The templates were also subjected to frameshift FASTx against GENPEPT, and homolog match was defined as having an E-value of ≦1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. No. 08/812,290 and U.S. Ser. No. 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis Mo.; http://pfam.wustl.edu/).

Templates were further analyzed using the bioinformatics tools described in the patents incorporated by reference herein, software applications such as MACDNASIS PRO software (Hitachi Software Engineering, South San Francisco Calif.) and LASERGENE software (DNASTAR), and queried against public databases such as the GenBank rodent, mammalian, vertebrate, prokaryote, and eukaryote databases, GenPept, SwissProt, BLOCKS, PRINTS, PFAM, and Prosite to acquire annotation.

The programs described above for the assembly and analysis of templates encoding electron transfer molecules were used to align the polynucleotides of the Sequence Listing with templates in LIFESEQ GOLD database (Incyte Pharmaceuticals). The templates that were annotated as electron transfer molecules provided the grouping and annotation of polynucleotides claimed herein as regulatory and coding sequences of a particular electron transfer molecule.

VII Chromosome Mapping

Radiation hybrid and genetic mapping data available from public resources such as the Stanford Human Genome Center (SHGC), Whitehead Institute for Genome Research (WIGR), and Genethon are used to determine if any of the polynucleotides presented in the Sequence Listing have been mapped. Any of the polynucleotides of a particular electron transfer molecule that have been mapped result in the assignment of all related regulatory and coding sequences of that electron transfer molecule mapping to the same location. The genetic map locations are described as ranges, or intervals, of human chromosomes. The map position of an interval, in centiMorgans (which is roughly equivalent to 1 megabase of human DNA), is measured relative to the terminus of the chromosome's p-arm.

VIII Hybridization Technologies and Analyses

### Immobilization of Polynucleotides on a Substrate

Polynucleotides are applied to a substrate by one of the following methods. A mixture of polynucleotides is fractionated by gel electrophoresis and transferred to a nylon membrane by capillary transfer. Alternatively, the polynucleotides are individually ligated to a vector and inserted into bacterial host cells to form a library. The polynucleotides are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37° C. for 16 hr. The membrane is removed from the agar and consecutively placed colony side up in 10% SDS, denaturing solution (1.5 M NaCl, 0.5 M NaOH ), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, polynucleotides are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (Amersham Pharmacia Biotech). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning, Acton Mass.) by ultrasound in 0.1% SDS and acetone, etching in 4% hydrofluoric acid (VWR Scientific Products, West Chester Pa.), coating with 0.05% aminopropyl silane (Sigma Aldrich) in 95% ethanol, and curing in a 110° C. oven. The slides are washed extensively with distilled water between and after treatments. The nucleic acids are arrayed onto the slide and then immobilized by exposing the array to UV irradiation using a STRATALINKERUV-crosslinker(Stratagene). Arrays are then washed at room temperature in 0.2% SDS and rinsed three times in distilled water. Non-specific binding sites are blocked by incubation of arrays in 0.2% casein in PBS (Tropix, Bedford Mass.) for 30 min at 60° C.; then the arrays are washed in 0.2% SDS and rinsed in distilled water as before.

### Probe Preparation for Membrane Hybridization

Hybridization probes derived from the polynucleotides of the Sequence Listing are employed for screening cDNAs, mRNAs, or genomic DNA in membrane-based hybridizations. Probes are prepared by diluting the polynucleotides to a concentration of 40-50 ng in 45 μl TE buffer, denaturing by heating to 100° C. for five min, and briefly centrifuging. The denatured polynucleotide is then added to a REDIPRIME tube (Amersham Pharmacia Biotech), gently mixed until blue color is evenly distributed, and briefly centrifuged. Five microliters of [³²P]dCTP is added to the tube, and the contents are incubated at 37° C. for 10 min. The labeling reaction is stopped by adding 5 μl of 0.2M EDTA, and probe is purified from unincorporated nucleotides using a PROBEQUANT G-50 microcolumn (Amersham Pharmacia Biotech). The purified probe is heated to 100° C. for five min, snap cooled for two min on ice, and used in membrane-based hybridizations as described below.

### Probe Preparation for Polymer Coated Slide Hybridization

Hybridization probes derived from mRNA isolated from samples are employed for screening polynucleotides of the Sequence Listing in array-based hybridizations. Probe is prepared using the GEMbright kit (Incyte Pharmaceuticals) by diluting mRNA to a concentration of 200 ng in 9 μl TE buffer and adding 5 μl 5×buffer, 1 μl 0.1 M DTT, 3 μl Cy3 or Cy5 labeling mix, 1 μRNase inhibitor, 1 μl reverse transcriptase, and 5 μl 1×yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37° C. for two hr. The reaction mixture is then incubated for 20 min at 85° C., and probes are purified using two successive CHROMA SPIN+TE 30 columns (Clontech). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800×g, and the pellet is resuspended in 12 μl resuspension buffer, heated to 65° C. for five min, and mixed thoroughly. The probe is heated and mixed as before and then stored on ice. Probe is used in high density array-based hybridizations as described below.

### Membrane-based Hybridization

Membranes are pre-hybridized in hybridization solution containing 1% Sarkosyl and 1×high phosphate buffer (0.5 M NaCl, 0.1 M Na₂HPO₄, 5 mM EDTA, pH 7) at 55° C. for two hr. The probe, diluted in 15 ml fresh hybridization solution, is then added to the membrane. The membrane is hybridized with the probe at 55° C. for 16 hr. Following hybridization, the membrane is washed for 15 min at 25° C. in 1 mM Tris (pH 8.0), 1% Sarkosyl, and four times for 15 min each at 25° C. in 1 mM Tris (pH 8.0). To detect hybridization complexes, XOMAT-AR film (Eastman Kodak, Rochester N.Y.) is exposed to the membrane overnight at −70° C., developed, and examined visually.

### Polymer Coated Slide-based Hybridization

Probe is heated to 65° C. for five min, centrifuged five min at 9400 rpm in a 5415C microcentrifuge (Eppendorf Scientific, Westbury N.Y.), and then 18 μl is aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60° C. The arrays are washed for 10 min at 45° C. in 1×SSC, 0.1% SDS, and three times for 10 min each at 45° C. in 0.1×SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of genes in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to substantially equal numbers of probes derived from both biological samples give a distinct combined fluorescence (Shalon WO95/35505).

Hybridization complexes are detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20×microscope objective (Nikon, Melville N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Pharmaceuticals).

IX Complementary Polynucleotides

Molecules complementary to the polynucleotide, or a fragment thereof, are used to detect, decrease, or inhibit gene expression. Although use of oligonucleotides comprising from about 15 to about 30 base pairs is described, the same procedure is used with larger or smaller fragments or their derivatives (e.g., PNAs). Appropriate oligonucleotides are selected using OLIGO 4.06 software (National Biosciences). To inhibit transcription by preventing promoter binding, a complementary oligonucleotide is designed to bind to the most unique 5′ sequence, most preferably about 10 nucleotides before the initiation codon of the open reading frame. To inhibit translation, a complementary oligonucleotide is designed to prevent ribosomal binding to the mRNA encoding the mammalian polypeptide.

In addition to using antisense molecules constructed to interrupt transcription or translation, modifications of gene expression can be obtained by designing antisense molecules to genomic sequences (such as enhancers or introns) or even to trans-acting regulatory genes. Similarly, antisense inhibition can be achieved using Hogeboom base-pairing methodology, also known as “triple helix” base pairing. Antisense molecules involved in triple helix pairing compromise the ability of the double helix to open sufficiently for the binding of polymerases, transcription factors, or regulatory molecules.

Such antisense molecules are placed in expression vectors and used to transform preferred cells or tissues. This may include introduction of the expression vector into a cell line to test efficacy; into an organ, tumor, synovial cavity, or the vascular system for transient or short term therapy; or into a stem cell or other reproducing lineage for long term or stable gene therapy. Transient expression may last for a month or more with a non-replicating vector and for three months or more if appropriate elements for inducing vector replication are used in the transformation/expression system.

Stable transformation of appropriate dividing cells with a vector encoding the antisense molecule can produce a transgenic cell line, tissue, or organism (U.S. Pat. No. 4,736,866). Those cells that assimilate and replicate sufficient quantities of the vector to allow stable integration also produce enough antisense molecules to compromise or entirely eliminate activity of the polynucleotide.

X Expression of the Mammalian Polypeptide

Expression and purification of the mammalian polypeptide are achieved using bacterial or virus-based expression systems. For expression in bacteria, cDNA is subcloned into a vector containing an antibiotic resistance gene and an inducible tac hybrid promoter in conjunction with the lac operator regulatory element that directs high levels of cDNA transcription. The recombinant vector is transformed into a BL21(DE3) bacterial host. Antibiotic resistant bacteria are induced with IPTG and express the mammalian polypeptide. Expression in eukaryotic cells is achieved by infecting Spodoptera frugiperda (Sf9) insect cells with recombinant baculovirus, Autographica californica nuclear polyhedrosis virus. The baculovirus polyhedrin gene is replaced with the mammalian cDNA by homologous recombination. The insect cells are infected with the virus, and the polyhedrin promoter drives high levels of expression.

For ease of purification, the mammalian polypeptide is synthesized as a fusion protein with glutathione-S-transferase (GST; Amersham Pharmacia Biotech) or a similar alternative such as FLAG. The fusion protein is purified on immobilized glutathione under conditions that maintain protein activity and antigenicity. After purification, the GST moiety is proteolytically cleaved from the mammalian polypeptide with thrombin. A fusion protein with FLAG, an 8-amino acid peptide, is purified using commercially available monoclonal and polyclonal anti-FLAG antibodies (Eastman Kodak, Rochester N.Y.).

XI Production of Antibodies

A denatured mammalian polypeptide from a reverse phase HPLC separation is obtained in quantities up to 75 mg. This denatured polypeptide is used to immunize mice or rabbits following standard protocols. About 100 μg is used to immunize a mouse, while up to 1 mg is used to immunize a rabbit. The denatured polypeptide is radioiodinated and incubated with murine B-cell hybridomas to screen for monoclonal antibodies. About 20 mg of polypeptide is sufficient for labeling and screening several thousand clones.

In another approach, the amino acid sequence translated from a polynucleotide of the invention is analyzed using PROTEAN software (DNASTAR) to determine regions of high immunogenicity. The optimal sequences for immunization are usually at the C-terminus, the N-terminus, and those intervening, hydrophilic regions of the polypeptide that are likely to be exposed to the external environment when the polypeptide is in its natural conformation. Typically, oligopeptides about 15 residues in length are synthesized using an ABI 431 Peptide synthesizer (PE Biosystems) using Fmoc-chemistry and then coupled to keyhole limpet hemocyanin (KLH; Sigma Aldrich) by reaction with M-maleimidobenzoyl-N-hydroxysuccinimide ester. If necessary, a cysteine may be introduced at the N-terminus of the peptide to permit coupling to KLH. Rabbits are immunized with the oligopeptide-KLH complex in complete Freund's adjuvant. The resulting antisera are tested for antipeptide activity by binding the peptide to plastic, blocking with 1% BSA, reacting with rabbit antisera, washing, and reacting with radioiodinated goat anti-rabbit IgG.

Hybridomas are prepared and screened using standard techniques. Hybridomas of interest are detected by screening with radioiodinated polypeptide to identify those fusions producing a particular electron transfer molecule-specific monoclonal antibody. In a typical protocol, wells of 96 well plates (FAST, Becton-Dickinson, Palo Alto Calif.) are coated with affinity-purified, specific rabbit-anti-mouse (or suitable anti-species Ig) antibodies at 10 mg/ml. The coated wells are blocked with 1% BSA and washed and exposed to supernatants from hybridomas. After incubation, the wells are exposed to radiolabeled polypeptide at 1 mg/ml. Clones producing antibodies bind a quantity of labeled polypeptide that is detectable above background.

Such clones are expanded and subjected to 2 cycles of cloning at 1 cell/3 wells. Cloned hybridomas are injected into pristane-treated mice to produce ascites, and monoclonal antibody is purified from the ascitic fluid by affinity chromatography on protein A (Amersham Pharmacia Biotech). Monoclonal antibodies with affinities of at least 10⁸ M⁻¹, preferably 10⁹ to 10¹⁰ M⁻¹ or stronger, are made by procedures well known in the art.

XII Purification of Polypeptide Using Specific Antibodies

Naturally occurring or recombinant mammalian polypeptide is substantially purified by immunoaffinity chromatography using antibodies specific for the polypeptide. An immunoaffinity column is constructed by covalently coupling the antibody to CNBr-activated SEPHAROSE resin (Amersham Pharmacia Biotech). Media containing the polypeptide is passed over the immunoaffinity column, and the column is washed using high ionic strength buffers in the presence of detergent to allow preferential absorbance of the polypeptide. After coupling, the polypeptide is eluted from the column using a buffer of pH 2-3 or a high concentration of urea or thiocyanate ion to disrupt antibody/polypeptide binding, and the polypeptide is collected.

XIII Screening Molecules or Compounds for Specific Binding

The polynucleotide, or fragments thereof, or the polypeptide, or portions thereof, are labeled with ³²P-dCTP, Cy3-dCTP, or Cy5-dCTP (Amersham Pharmacia Biotech), or with BIODIPY or FITC (Molecular Probes), respectively. A library or a plurality of candidate molecules or compounds previously arranged on a substrate are incubated in the presence of labeled polynucleotide or polypeptide. After incubation under conditions for a polynucleotide or polypeptide, the substrate is washed. Any position on the substrate retaining label, that indicates specific binding or complex formation, identifies a ligand. Data obtained using different concentrations of the polynucleotide or polypeptide are used to calculate affinity between the labeled polynucleotide or polypeptide and the bound ligand.

XIV Two-Hybrid Screen

A yeast two-hybrid system, MATCHMAKER LexA Two-Hybrid system (Clontech Laboratories, Palo Alto Calif.), is used to screen for peptides that bind the mammalian polypeptide of the invention. A polynucleotide encoding the polypeptide is inserted into the multiple cloning site of a pLexA vector, ligated, and transformed into E. coli. cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48 [p8op-lacZ] cells using a polyethylene glycol/lithium acetate protocol. Transformed yeast cells are plated on synthetic dropout (SD) media lacking histidine (-His), tryptophan (-Trp), and uracil (-Ura), and incubated at 30° C. until the colonies have grown up and can be counted. The colonies are pooled in a minimal volume of 1×TE (pH 7.5), replated on SD/-His/-Leu/-Trp/-Ura media supplemented with 2% galactose (Gal), 1% raffinose (Raf), and 80 mg/ml 5-bromo-4-chloro-3-indolyl β-galactopyranoside (X-Gal), and subsequently examined for growth of blue colonies. Interaction between expressed polypeptide and cDNA fusion proteins activates expression of a LEU2 reporter gene in EGY48 and produces colony growth on media lacking leucine (−Leu). Interaction also activates expression of β-galactosidase from the p8op-lacZ reporter construct that produces blue color in colonies grown on X-Gal.

Positive interactions between expressed polypeptide and cDNA fusion proteins are verified by isolating individual positive colonies and growing them in SD/-Trp/-Ura liquid medium for 1 to 2 days at 30° C. A sample of the culture is plated on SD/-Trp/-Ura media and incubated at 30° C. until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gal/-Trp/-Ura, and white colonies are isolated and propagated. The pB42AD-cDNA plasmid, which contains a polynucleotide encoding a protein that physically interacts with the mammalian polypeptide, can be isolated from the yeast cells and characterized.

XV Gene Transcript Analysis

The abundance sort program of the invention described in U.S. Pat. No. 5,840,484, tabulates and sorts by frequency the mRNA transcripts corresponding to each gene identified in a database. The process for obtaining this final data set, the profile of gene activity or transcript image, is referred to as “gene transcript analysis”.

A transcript analysis summarizes the presence and abundance of exact, unique, and homologous transcripts that are tissue specific. Such a collection of transcripts can help distinguish between normal tissue and that biopsied from a subject displaying a particular disorder. Comparison of these profiles identify polynucleotides that are not active, minimally active, or overactive. The RNA or expressed polypeptide is a candidate for intervention in the disease process. Where polynucleotide expression was absent, a therapeutic supplying the missing molecule would be preferred. In cases of underexpression, the polynucleotide or its product (mRNA or polypeptide) could also be supplied exogenously. Where a polynucleotide was overexpressed, antisense or inhibiting molecules or compounds are deemed appropriate. During or following treatment, observation of desired phenotypic changes and transcript images help determine whether therapeutic intervention is restoring or has restored the normal profile.

XVI Functional Assays

The activity of an electron transfer molecule is generally determined by measuring the change in the spectral absorbance of the prosthetic group of the molecule at a wavelength characteristic of its conversion between an oxidized and a reduced state in the presence of a suitable oxidoreductase enzyme and a secondary electron donor/acceptor, such as NAD+/NADH. The reduction of cytochrome b5 in the presence of NADH and NADH:cytochrome b5 reductase is presented as an example. Cytochrome b5 and NADH:cytochrome b5 reductase are incubated together in a suitable buffer, and the reaction is initiated by addition of NADH. The reaction is carried out in a glass cuvette incubated at 30-37° C. and is monitored over a period of time in a visible spectrophotometer at 565 nm. The rate of change in absorption measured at this wavelength is due to the reduction of the heme group in cytochrome b5 and is proportional to the activity of cytochrome b5 present.

All patents and publications mentioned in the specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

TABLE 1

| SEQ ID | INCYTE ID | | | | |
|---|---|---|---|---|---|
| NO | NO | CLONE | TEMPLATE | LIBRARY | FILING HISTORY (DOCKET NO;SEQ ID NO;FILING DATE) |
| 1 | hu01031982 | 5564221 | LG:350517.1 | BRSTDIT01 | |
| 2 | hu00713471 | 2266979 | IB:076637.1 | UTRSNOT02 | PD-0288-P,575,12/18/1996\|PD-0288-US,575,12/16/1997 |
| 3 | hu01170046 | 6422174 | LG:240805.4 | BRSTUNT01 | |
| 4 | hu01180073 | 6413044 | LG:240805.4 | UTREDIT10 | |
| 5 | hu01201763 | 6180012 | LG:240805.4 | BMARUNT02 | |
| 6 | hu01322950 | 5884355 | LG:240805.4 | LIVRNON08 | |
| 7 | hu01330729 | 6325092 | LG:240805.4 | LUNGDIN02 | |
| 8 | hu00983306 | 6377218 | LG:240805.4 | FIBAUNT01 | |
| 9 | hu01239428 | 5886010 | LG:240805.4 | LIVRNON08 | |
| 10 | hu01185563 | 6421181 | LG:240805.4 | BRSTUNT01 | |
| 11 | hu01313867 | 6479549 | LG:240805.4 | PROSTMC01 | |
| 12 | hu01140406 | 5949878 | LG:240805.4 | LIVRTUN04 | |
| 13 | hu01228388 | 5656380 | LG:240805.4 | BSCNNOT03 | |
| 14 | hu00981727 | 6325013 | LG:240805.4 | LUNGDIN02 | |
| 15 | hu01186567 | 6358853 | LG:240805.4 | CONFNOT09 | |
| 16 | hu01122732 | 5844535 | LG:240805.4 | BRAENOT04 | |
| 17 | hu01012436 | 5994013 | LG:244681.1 | FTUBTUT02 | |
| 18 | hu01002677 | 6419669 | LG:037054.7 | BRSTUNT01 | |
| 19 | hu00978833 | 3549771 | LG:234480.6 | BRONDIT01 | |
| 20 | hu01284599 | 6030694 | LG:234480.6 | BRAHNON05 | |
| 21 | hu01086979 | 4519787 | LG:234480.6 | SINJNOT03 | |
| 22 | hu01032083 | 6111313 | LG:234480.6 | MCLDTXT03 | |
| 23 | hu01210547 | 4437887 | LG:234480.6 | SINTNOT22 | |
| 24 | hu01123095 | 5911025 | LG:234480.6 | BRAIFEN05 | |
| 25 | hu01329043 | 6152806 | LG:234480.6 | ENDMUNT04 | |
| 26 | hu01116805 | 6119359 | LG:234480.6 | BRAHNON05 | |
| 27 | hu01102991 | 5714759 | LG:234480.6 | MASTTXT01 | |
| 28 | hu01013649 | 6099952 | LG:234480.6 | UTRENOT09 | |
| 29 | hu01243383 | 6209977 | LG:234480.6 | ARTANOT06 | |
| 30 | hu01293306 | 3153065 | LG:234480.6 | TLYMTXT02 | |
| 31 | hu01104919 | 4437787 | LG:234480.6 | SINTNOT22 | |
| 32 | hu01329433 | 4442819 | LG:234480.6 | SINTNOT22 | |
| 33 | hu00986013 | 6378670 | LG:234480.6 | FIBAUNT01 | |
| 34 | hu01078335 | 6015470 | LG:234480.6 | FIBRUNT02 | |
| 35 | hu01034993 | 4517716 | LG:234480.6 | SINJNOT03 | |
| 36 | hu01332579 | 6383466 | LG:234480.6 | FIBRUNT02 | |
| 37 | hu01267654 | 6495789 | LG:234480.6 | COLNNOT41 | |
| 38 | hu01175359 | 5811756 | LG:234480.6 | KIDCTMT02 | |
| 39 | hu01193983 | 3901675 | LG:234480.6 | LUNGNON03 | |
| 40 | hu00755546 | 523345 | IB:779812.1 | MMLR2DT01 | PD-0044-P,2538,10/02/1995\|PD-0044-US,2538,10/02/1996 |
| 41 | hu00668597 | 2024316 | IB:816379.1 | KERANOT02 | PD-0260-P,104,10/25/1996\|PD-0116-1-US,1526,10/24/1997 |
| 42 | hu00899952 | 2663830 | IB:816379.1 | ADRENOT08 | PD-0317-P,1069,02/11/1997\|PD-0317-US,1069,02/10/1998 |
| 43 | hu00504101 | 2069960 | IB:816379.1 | ISLTNOT01 | PD-0259-P,373,10/28/1996\|PD-0259-US,373,10/28/1997 |
| 44 | hu00090502 | 2850044 | IB:816379.1 | BRSTTUT13 | PD-0336-P,806,03/18/1997 |
| 45 | hu00902140 | 2484903 | IB:816379.1 | BONRTUT01 | PD-0303-P,1777,01/27/1997 |
| 46 | hu00503171 | 1493337 | IB:816379.1 | PROSNON01 | PD-0198-P,493,07/29/1996\|PD-0198-US,5100,07/28/1997 |
| 47 | hu00046235 | 664658 | IB:816379.1 | SCORNOT01 | PD-0094-P,461,12/14/1995\|PD-0094-US,461,12/12/1996 |
| 48 | hu00007392 | 386476 | IB:816379.1 | THYMNOT02 | PD-0041-P,2075,09/26/1995\|PD-0077-P,2075,10/02/1995\|PD-0077- |
| | | | | | US,2075,10/02/1996 |
| 49 | hu00381091 | 488003 | IB:816379.1 | HNT2AGT01 | |
| 50 | hu00562460 | 2634306 | IB:816379.1 | COLNTUT15 | PD-0305-P,1665,01/22/1997\|PD-0305-US,1665,01/22/1998 |
| 51 | hu00039191 | 2318093 | IB:816379.1 | OVARNOT02 | PD-0045-1-US,2387,11/10/1997\|PD-0293-P,560,12/13/1996 |
| 52 | hu00605418 | 611497 | IB:816379.1 | COLNNOT01 | PD-0059-P,1003,11/10/1995\|PD-0059-US,1003,11/08/1996 |
| 53 | hu00725335 | 873135 | IB:816379.1 | LUNGAST01 | PD-0120-US,390,02/28/1997\|PD-0124-P,390,03/13/1996 |
| 54 | hu00651653 | 2367584 | IB:816379.1 | ADRENOT07 | PD-0301-P,1460,12/19/1996\|PD-0301-US,1460,12/17/1997 |
| 55 | hu00916613 | 57744 | IB:816379.1 | | PD-0009-US,257,07/08/1994 |
| 56 | hu00814652 | 621001 | IB:816379.1 | PGANNOT01 | PD-0062-P,1272,11/22/1995\|PD-0062-US,1272,11/22/1996 |
| 57 | hu00163808 | 1260150 | IB:816379.1 | MENITUT03 | PD-0147-P,2094,04/17/1996\|PD-0147-US,2094,04/17/1997 |
| 58 | hu00687420 | 2365110 | IB:816379.1 | ADRENOT07 | PD-0301-P,592,12/19/1996\|PD-0301-US,592,12/17/1997 |
| 59 | hu00238960 | 2364628 | IB:816379.1 | ADRENOT07 | PD-0301-P,415,12/19/1996\|PD-0301-US,415,12/17/1997 |
| 60 | hu00776351 | 2869942 | IB:816379.1 | THYRNOT10 | PD-0395-P,704,07/02/1997\|PD-0395-US,704,06/23/1998 |
| 61 | hu00883244 | 2858353 | IB:816379.1 | SININOT03 | PD-0343-P,92,03/25/1997\|PD-0343-US,92,03/25/1998 |
| 62 | hu00816546 | 3034616 | IB:816379.1 | TLYMNOT05 | PD-0364-P,1406,05/02/1997\|PD-0364-US,1406,04/30/1998 |
| 63 | hu00861826 | 621158 | IB:816379.1 | PGANNOT01 | PD-0062-P,1340,11/22/1995\|PD-0062-US,1340,11/22/1996 |
| 64 | hu00187718 | 2632061 | IB:816379.1 | COLNTUT15 | PD-0305-P,854,01/22/1997\|PD-0305-US,854,01/22/1998 |
| 65 | hu00681549 | 640040 | IB:816379.1 | BRSTNOT03 | PD-0061-US,946,11/15/1996\|PD-0065-P,946,11/28/1995 |
| 66 | hu00339586 | 2841649 | IB:816379.1 | DRGLNOT01 | PD-0337-P,1803,03/18/1997\|PD-0337-US,1803,03/05/1998 |
| 67 | hu01022298 | 6057485 | LG:171764.2 | BRAENOT04 | |
| 68 | hu01122008 | 4125481 | LG:234480.7 | BRSTTUT26 | |
| 69 | hu00228730 | 2965517 | IB:815588.1 | SCORNOT04 | PD-0360-P,968,04/24/1997\|PD-0360-US,968,04/23/1998 |
| 70 | hu01259066 | 6208681 | LG:234480.8 | ARTANOT06 | |
| 71 | hu01271939 | 6075669 | LG:234480.8 | UTREDIT09 | |
| 72 | hu01144669 | 6566164 | LG:955064.1 | MCLDTXT04 | |
| 73 | hu01312454 | 6271522 | LG:956415.1 | BRAIFEN03 | |
| 74 | hu00986823 | 5560063 | LG:214288.6 | BRSTDIT01 | |
| 75 | hu01207133 | 5561495 | LG:214288.6 | BRSTDIT01 | |
| 76 | hu01060525 | 6433941 | LG:214288.6 | LUNGNON07 | |
| 77 | hu01108730 | 6456026 | LG:214288.6 | COLNDIC01 | |
| 78 | hu01094006 | 4121704 | LG:214288.6 | BRSTTUT25 | |
| 79 | hu01055702 | 6137123 | LG:214288.6 | BMARTXT02 | |
| 80 | hu01200549 | 5656338 | LG:214288.6 | BSCNNOT03 | |
| 81 | hu01256253 | 6137155 | LG:214288.6 | BMARTXT02 | |
| 82 | hu01029382 | 5943751 | LG:214288.6 | COLADIT05 | |
| 83 | hu01009078 | 6380182 | LG:214288.6 | FIBRUNT02 | |
| 84 | hu01321595 | 6409908 | LG:214288.6 | UTREDIT10 | |
| 85 | hu01164609 | 5697069 | LG:214288.6 | BRSTTUT13 | |
| 86 | hu01253674 | 3567727 | LG:214288.6 | BRONNOT02 | |
| 87 | hu01169353 | 6507225 | LG:214288.6 | BRAHNOT02 | |
| 88 | hu01206885 | 6389422 | LG:214288.6 | MIXDUNB01 | |
| 89 | hu01348582 | 5810417 | LG:214288.6 | KIDCTMT02 | |
| 90 | hu01338934 | 6522760 | LG:214288.6 | CONFTDT02 | |
| 91 | hu01164479 | 5646107 | LG:214288.6 | BRAITUT23 | |
| 92 | hu01287961 | 6397064 | LG:214288.6 | UTRENOT10 | |
| 93 | hu01236008 | 5943406 | LG:348773.1 | COLADIT05 | |
| 94 | rat00086191 | 700294566 | ZS:210500.2 | RABFNOT02 | PZ-0013-P,872,08/08/1997\|PZ-0013-US,872,08/07/1998 |
| 95 | rat00096533 | 700309980 | ZS:210500.2 | RASDNOT02 | PZ-0028-P,1049,09/08/1997\|PZ-0027-US,2303,09/08/1998 |
| 96 | rat00191537 | 700933938 | ZS:210500.2 | RALINON03 | PZ-0075-P,1526,03/10/1998\|PZ-0075-US,2466,03/09/1999 |
| 97 | rat00075375 | 701904240 | ZS:210500.2 | RABYUNS09 | PZ-0131-P,4285,06/29/1999 |
| 98 | rat00219672 | 701231886 | ZS:210500.2 | RASJNON03 | PZ-0098-P,1420,11/12/1998 |
| 99 | rat00223885 | 700127612 | ZS:210500.2 | RABHNOT01 | PZ-0004-P,2491,07/09/1997\|PZ-0004-US,2491,07/09/1998 |
| 100 | rat00247348 | 701828746 | ZS:210500.2 | RAKITXT09 | PZ-0132-P,973,08/10/1999 |
| 101 | rat00069166 | 701339683 | ZS:210500.2 | RALITXT08 | PZ-0092-P,1343,08/13/1998 |
| 102 | rat00038669 | 701439922 | ZS:210500.2 | RALINON08 | PZ-0113-P,2219,04/07/1999 |
| 103 | rat00205020 | 700483806 | ZS:210500.2 | RALINON03 | PZ-0059-P,1650,01/16/1998\|PZ-0059-US,1650,01/12/1999 |
| 104 | rat00000073 | 700933808 | ZS:210500.2 | RALINON03 | PZ-0075-P,1477,03/10/1998\|PZ-0075-US,2417,03/09/1999 |
| 105 | rat00221180 | 700635387 | ZS:210500.2 | RATONOT01 | PZ-0036-US,145,10/09/1998 |
| 106 | rat00008019 | 701186639 | ZS:210500.2 | RACONON02 | PZ-0085-P,177,06/11/1998 |
| 107 | rat00033426 | 700067896 | ZS:210500.2 | RABTNOT01 | PZ-0001-P,1401,06/12/1997\|PZ-0001-US,1401,06/08/1998 |
| 108 | rat00131752 | 701654036 | ZS:210500.2 | RALITXT10 | PZ-0117-P,1385,05/07/1999 |
| 109 | rat00230675 | 701616311 | ZS:210500.2 | RALITXT61 | PZ-0127-P,156,07/07/1999 |
| 110 | rat00056283 | 700123547 | ZS:210500.2 | RABHNOT01 | PZ-0004-P,721,07/09/1997\|PZ-0004-US,721,07/09/1998 |
| 111 | rat00144276 | 701727660 | ZS:210500.2 | RALITXT1A | PZ-0122-P,257,06/03/1999 |
| 112 | hu01337947 | 5996159 | LG:999424.1 | FTUBTUT02 | |
| 113 | hu01310032 | 3786708 | LG:999424.1 | BRAHNOT05 | |
| 114 | hu00987948 | 4438637 | LG:999262.1 | SINTNOT22 | |
| 115 | hu01216237 | 3020423 | LG:999262.1 | PROSDIN01 | |
| 116 | hu01032720 | 4122257 | LG:999262.1 | BRSTTUT25 | |
| 117 | hu01119107 | 5479570 | LG:899715.1 | FIBPFEN06 | |
| 118 | hu01347050 | 5479760 | LG:899715.1 | FIBPFEN06 | |
| 119 | hu01343361 | 5479960 | LG:899715.1 | FIBPFEN06 | |
| 120 | hu01104646 | 5480114 | LG:899715.1 | FIBPFEN06 | |
| 121 | hu01179389 | 5473892 | LG:899715.1 | FIBPFEN06 | |
| 122 | hu00969772 | 5474141 | LG:899715.1 | FIBPFEN06 | |
| 123 | hu00994747 | 5475667 | LG:899715.1 | FIBPFEN06 | |
| 124 | hu01209376 | 5475060 | LG:899715.1 | FIBPFEN06 | |
| 125 | hu01330016 | 5481168 | LG:899715.1 | FIBPFEN06 | |
| 126 | hu01175881 | 5480121 | LG:899715.1 | FIBPFEN06 | |
| 127 | hu01264927 | 5477881 | LG:899715.1 | FIBPFEN06 | |
| 128 | hu01121470 | 5476185 | LG:899715.1 | FIBPFEN06 | |
| 129 | hu00996037 | 5474878 | LG:899715.1 | FIBPFEN06 | |
| 130 | hu01052325 | 5476523 | LG:899715.1 | FIBPFEN06 | |
| 131 | hu01137559 | 6597357 | LG:999424.4 | ARTANOT06 | |
| 132 | hu01092065 | 6597429 | LG:999424.4 | ARTANOT06 | |
| 133 | hu01323636 | 3787304 | LG:999424.4 | BRAHNOT05 | |
| 134 | hu01060728 | 6597257 | LG:999424.4 | ARTANOT06 | |
| 135 | hu01268836 | 6118845 | LG:999424.4 | BRAHNON05 | |
| 136 | hu01222473 | 6298190 | LG:230151.1 | UTREDIT07 | |
| 137 | hu01236994 | 6138222 | LG:230151.1 | BMARTXT03 | |
| 138 | hu00308470 | 1849578 | IB:234480.2 | LUNGFET03 | PD-0230-P,450,08/23/1996\|PD-0230-US,450,08/22/1997 |
| 139 | hu00267375 | 661163 | IB:234480.2 | BRAINOT03 | PD-0088-P,1425,11/29/1995\|PD-0088-US,3170,11/27/1996 |
| 140 | hu00473030 | 2101509 | IB:234480.2 | BRAITUT02 | PD-0111-1-US,5693,12/05/1997\|PD-0275-P,6153,12/06/1996 |
| 141 | hu00173475 | 2923187 | IB:234480.3 | SININOT04 | PD-0344-P,810,03/17/1997\|PD-0344-US,810,03/16/1998 |
| 142 | hu00849020 | 2052778 | IB:234480.3 | LIVRFET02 | PD-0262-P,1216,10/31/1996 |
| 143 | hu00168009 | 2347967 | IB:234480.3 | COLSUCT01 | PD-0278-P,56,12/05/1996\|PD-0278-US,56,11/21/1997 |
| 144 | hu00580866 | 892747 | IB:234480.3 | STOMTUT01 | PD-0127-P,1131,03/08/1996 |
| 145 | hu00927748 | 154668 | IB:240805.5 | THP1PLB02 | |
| 146 | hu00403005 | 2473237 | IB:240805.5 | THP1NOT03 | PD-0286-P,1246,11/25/1996\|PD-0286-US,1246,11/04/1997 |
| 147 | hu00869778 | 2173121 | IB:240805.5 | ENDCNOT03 | PD-0279-P,1788,11/22/1996\|PD-0279-US,1788,11/07/1997 |
| 148 | hu00063607 | 2048670 | IB:240805.5 | LIVRFET02 | PD-0262-P,158,10/31/1996 |
| 149 | hu00203909 | 1289023 | IB:240805.5 | BRAINOT11 | PD-0156-P,985,04/25/1996\|PD-0156-US,985,04/25/1997 |
| 150 | hu00452472 | 1997754 | IB:240805.5 | BRSTTUT03 | PD-0241-P,1221,10/01/1997\|PD-0143-1-US,5251,10/01/1997 |
| 151 | hu00831183 | 55605 | IB:240805.5 | | PD-0008-US,1564,06/27/1994 |
| 152 | hu00372084 | 2293016 | IB:240805.5 | BRAINON01 | PD-0274-P,5097,11/20/1996 |
| 153 | hu00796320 | 1888743 | IB:240805.5 | BLADTUT07 | PD-0238-P,1903,09/24/1996\|PD-0238-US,1903,09/23/1997 |
| 154 | hu00613827 | 1700085 | IB:240805.5 | BLADTUT05 | PD-0202-P,1281,07/31/1996\|PD-0221-US,874,07/31/1997 |
| 155 | hu00653184 | 2246684 | IB:240805.5 | HIPONON02 | PD-0258-P,3714,03/05/1997\|PD-0258-US,3714,02/27/1998 |
| 156 | hu00533558 | 1243521 | IB:240805.5 | LUNGNOT03 | PD-0158-P,929,04/22/1996 |
| 157 | hu00578046 | 1682120 | IB:240805.5 | PROSNOT15 | PD-0200-P,11,07/24/1996\|PD-0200-US,11,07/24/1997 |
| 158 | hu00482792 | 519530 | IB:240805.5 | MMLR2DT01 | PD-0044-P,1486,10/02/1995\|PD-0044-US,1486,10/02/1996 |
| 159 | hu00471785 | 674959 | IB:240805.5 | CRBLNOT01 | PD-0089-US,2305,11/27/1996\|PD-0093-P,609,12/18/1995 |
| 160 | hu00335877 | 2246604 | IB:240805.5 | HIPONON02 | PD-0258-P,3670,03/05/1997\|PD-0258-US,3670,02/27/1998 |
| 161 | hu00527595 | 1450183 | IB:240805.5 | PLACNOT02 | PD-0193-P,1110,06/27/1996\|PD-0167-US,2470,05/29/1997 |
| 162 | hu00875185 | 676179 | IB:240805.5 | CRBLNOT01 | PD-0093-P,1034,12/18/1995\|PD-0089-US,2730,11/27/1996 |
| 163 | hu00912110 | 1603276 | IB:240805.5 | LUNGNOT15 | PD-0208-P,2989,07/24/1996\|PD-0208-US,2989,07/23/1997 |
| 164 | hu00026601 | 735309 | IB:240805.5 | TONSNOT01 | PD-0100-P,511,01/05/1996\|PD-0100-US,511,01/03/1997 |
| 165 | hu00103248 | 1853346 | IB:240805.5 | LUNGFET03 | PD-0230-P,1981,08/23/1996\|PD-0230-US,1981,08/22/1997 |
| 166 | hu00631611 | 1728347 | IB:240805.5 | PROSNOT14 | PD-0201-P,1827,07/29/1996\|PD-0201-US,1827,07/28/1997\|PD-0210 |
| | | | | | P,1827,07/24/1996 |
| 167 | hu00401675 | 2347508 | IB:240805.5 | TESTTUT02 | PD-0285-P,1544,11/21/1996\|PD-0164-US,3026,05/15/1997 |
| 168 | hu00676022 | 770596 | IB:240805.5 | COLNCRT01 | PD-0110-US,1685,01/30/1997\|PD-0110-P,340,01/30/1996 |
| 169 | hu00950091 | 269698 | IB:240805.5 | HNT2NOT01 | PD-0038-P,1027,06/16/1995 |
| 170 | hu00780712 | 2345639 | IB:240805.5 | TESTTUT02 | PD-0164-US,2438,05/15/1997\|PD-0285-P,956,11/21/1996 |
| 171 | hu00392614 | 2313282 | IB:240805.5 | NGANNOT01 | PD-0297-P,2873,12/18/1996\|PD-0297-US,2873,12/17/1997 |
| 172 | hu00407933 | 536888 | IB:240805.5 | LNODNOT02 | PD-0060-P,131,10/30/1995 |
| 173 | hu00759320 | 579434 | IB:240805.5 | BRAVTXT05 | PR-0003-US,1863,11/13/1996\|PR-0004-P,1863,12/15/1995 |
| 174 | hu00709083 | 809810 | IB:240805.5 | LUNGNOT04 | PD-0121-P,360,02/29/1996\|PD-0121-US,360,02/28/1997 |
| 175 | hu00848224 | 2903460 | IB:240805.5 | DRGCNOT01 | PD-0337-US,6086,03/05/1998 |
| 176 | hu00703783 | 980041 | IB:240805.5 | TONGTUT01 | PD-0145-P,169,04/15/1996\|PD-0145-US,169,04/15/1997 |
| 177 | hu00805276 | 158997 | IB:240805.5 | ADENINB01 | PD-0033-1-US,290,07/13/1995 |
| 178 | hu00921782 | 3468653 | IB:240805.5 | BRAIDIT01 | PD-0384-P,972,06/06/1997\|PD-0384-US,972,06/04/1998 |
| 179 | hu00335487 | 2497291 | IB:240805.5 | ADRETUT05 | PD-0318-P,1870,02/11/1997\|PD-0318-US,1870,02/09/1998 |
| 180 | hu00549507 | 820929 | IB:240805.5 | KERANOT02 | PD-0116-1-US,713,10/24/1997\|PD-0116-P,713,02/26/1996\|PD-0116- |
| | | | | | US,713,02/26/1997 |
| 181 | hu00888223 | 767530 | IB:240805.5 | LUNGNOT04 | PD-0140-P,1354,04/10/1996\|PD-0121-US,2801,02/28/1997 |
| 182 | hu00224887 | 855022 | IB:240805.5 | NGANNOT01 | PD-0123-US,1166,02/28/1997\|PD-0125-P,1166,03/07/1996 |
| 183 | hu00935744 | 32873 | IB:240805.5 | THP1NOB01 | PD-0001-3-US,6764,02/14/1994\|PD-0001-4-US,6934,07/28/1994 |
| 184 | hu00861918 | 911244 | IB:240805.5 | STOMNOT02 | PD-0155-P,504,04/26/1996\|PD-0155-US,504,04/25/1997 |
| 185 | hu00534975 | 1913132 | IB:240805.5 | PROSTUT04 | PD-0197-US,3473,06/23/1997\|PD-0232-P,905,09/05/1996 |
| 186 | hu00031360 | 772404 | IB:240805.5 | COLNCRT01 | PD-0110-US,2014,01/30/1997\|PD-0110-P,669,01/30/1996 |
| 187 | hu00281501 | 3115741 | IB:240805.5 | LUNGTUT13 | PD-0358-P,1952,04/18/1997\|PD-0358-US,1952,04/17/1998 |
| 188 | hu00128107 | 1750586 | IB:240805.5 | STOMTUT02 | PD-0235-P,1383,08/30/1996 |
| 189 | hu00272072 | 2259695 | IB:240805.5 | OVARTUT01 | PD-0283-P,3754,11/21/1996\|PD-0112-1-US,5614,11/14/1997 |
| 190 | hu00651107 | 2913628 | IB:240805.5 | KIDNTUT15 | |
| 191 | hu00930901 | 270440 | IB:240805.5 | HNT2NOT01 | PD-0038-P,1258,06/16/1995 |
| 192 | hu00271706 | 2510064 | IB:240805.5 | CONUTUT01 | PD-0300-P,3179,12/20/1996\|PD-0300-US,3179,12/18/1997 |
| 193 | hu00008916 | 1611520 | IB:240805.5 | COLNTUT06 | PD-0206-P,3505,08/01/1996\|PD-0206-US,3505,08/01/1997 |
| 194 | hu00405925 | 30512 | IB:240805.5 | THP1NOB01 | PD-0001-3-US,5057,02/14/1994\|PD-0001-4-US,5227,07/28/1994 |
| 195 | hu00283922 | 1557831 | IB:240805.5 | BLADTUT04 | PD-0203-P,4507,08/01/1996\|PD-0203-US,4507,08/01/1997 |
| 196 | hu00394641 | 481021 | IB:240805.5 | LIVRBCT01 | PD-0079-P,188,10/04/1995\|PD-0079-US,188,10/04/1996 |
| 197 | hu00741298 | 2116071 | IB:240805.5 | BRSTTUT02 | PD-0255-P,219,10/23/1996 |
| 198 | hu00509990 | 989350 | IB:240805.5 | LVENNOT03 | PD-0134-P,1564,03/21/1996\|PD-0134-US,1564,03/21/1997 |
| 199 | hu00391975 | 1806228 | IB:240805.5 | SINTNOT13 | PD-0234-P,912,08/28/1996 |
| 200 | hu00681668 | 882564 | IB:240805.5 | THYRNOT02 | PD-0122-P,1602,02/29/1996\|PD-0122-US,1602,02/28/1997 |
| 201 | hu00157699 | 2379423 | IB:240805.5 | ISLTNOT01 | PD-0294-P,1550,12/20/1996\|PD-0259-US,4650,10/28/1997 |
| 202 | hu00240223 | 994052 | IB:240805.5 | COLNNOT11 | PD-0129-P,2380,03/19/1996\|PD-0129-US,2380,03/18/1997 |
| 203 | hu00842100 | 880888 | IB:240805.5 | THYRNOT02 | PD-0122-P,954,02/29/1996\|PD-0122-US,954,02/28/1997 |
| 204 | hu00533420 | 764170 | IB:240805.5 | LUNGNOT04 | PD-0140-P,156,04/10/1996\|PD-0121-US,1603,02/28/1997 |
| 205 | hu00211556 | 1564580 | IB:240805.5 | HEALDIT02 | PR-0007-P,317,06/26/1996\|PR-0007-US,317,06/25/1997 |
| 206 | hu00391700 | 2170220 | IB:240805.5 | ENDCNOT03 | PD-0279-P,459,11/22/1996\|PD-0279-US,459,11/07/1997 |
| 207 | hu00622361 | 1898981 | IB:240805.5 | BLADTUT06 | PD-0238-P,32,09/24/1996\|PD-0238-US,32,09/23/1997 |
| 208 | hu00629111 | 2262145 | IB:240805.5 | UTRSNOT02 | PD-0288-P,2429,12/18/1996\|PD-0288-US,2429,12/16/1997 |
| 209 | hu00757004 | 1290739 | IB:240805.5 | BRAINOT11 | PD-0156-P,1694,04/25/1996\|PD-0156-US,1694,04/25/1997 |
| 210 | hu00382550 | 692787 | IB:240805.5 | LUNGTUT02 | PD-0097-P,1386,12/20/1995\|PD-0097-US,1386,12/20/1996 |
| 211 | hu00516056 | 1650650 | IB:240805.5 | PROSTUT09 | PD-0199-P,3877,07/30/1996 |
| 212 | hu00268304 | 56716 | IB:240805.5 | | PD-0008-US,2105,06/27/1994 |
| 213 | hu00878200 | 1856963 | IB:240805.5 | PROSNOT18 | PD-0233-P,443,09/05/1996\|PD-0233-US,443,09/05/1997 |
| 214 | hu00235276 | 3028959 | IB:240805.5 | HEARFET02 | PD-0353-P,1179,04/18/1997\|PD-0353-US,1179,03/17/1998 |
| 215 | hu00652761 | 1580413 | IB:240805.5 | DUODNOT01 | PD-0207-P,711,07/29/1996\|PD-0207-US,711,07/29/1997 |
| 216 | hu00008766 | 508309 | IB:240805.5 | TMLR3DT02 | PD-0081-P,1795,10/25/1995\|PD-0081-US,1795,10/24/1996 |
| 217 | hu00076745 | 2695558 | IB:240805.5 | UTRSNOT12 | PD-0346-US,4896,03/27/1998\|PD-0346-P,618,03/27/1997 |
| 218 | hu00177708 | 1661051 | IB:240805.5 | URETTUT01 | PD-0216-P,1454,07/31/1996\|PD-0216-US,1454,07/30/1997 |
| 219 | hu00588913 | 693014 | IB:240805.5 | LUNGTUT02 | PD-0097-P,1459,12/20/1995\|PD-0097-US,1459,12/20/1996 |
| 220 | hu01070024 | 6413730 | LG:232035.2 | UTREDIT10 | |
| 221 | hu00173405 | 3927591 | IB:234480.4 | KIDNNOT19 | |
| 222 | hu00039514 | 219065 | IB:234480.4 | STOMNOT01 | PD-0022-US,2887,02/07/1995 |
| 223 | hu00026942 | 217054 | IB:234480.4 | STOMNOT01 | PD-0022-US,1753,02/07/1995 |
| 224 | hu00187054 | 215070 | IB:234480.4 | STOMNOT01 | PD-0022-US,644,02/07/1995 |
| 225 | hu00854314 | 219390 | IB:234480.4 | STOMNOT01 | PD-0022-US,3122,02/07/1995 |
| 226 | hu00885122 | 218904 | IB:234480.4 | STOMNOT01 | PD-0022-US,2751,02/07/1995 |
| 227 | hu00217359 | 77197 | IB:234480.4 | SYNORAB01 | PD-0012-US,149,08/19/1994 |
| 228 | hu00384526 | 3928041 | IB:234480.4 | KIDNNOT19 | |
| 229 | rat00071677 | 700532674 | ZS:217274.1 | RAEONOT02 | PZ-0024-US,242,09/10/1998\|PZ-0052-P,242,12/11/1997 |
| 230 | rat00105537 | 700520955 | ZS:217274.1 | RABHNOT02 | PZ-0021-US,2309,09/08/1998\|PZ-0050-P,2309,12/11/1997 |
| 231 | rat00177349 | 700785531 | ZS:217274.1 | RABSNOT02 | PZ-0057-P,2193,01/16/1998\|PZ-0057-US,2193,01/11/1999 |
| 232 | rat00068188 | 700525644 | ZS:217274.1 | RABMNOT01 | PZ-0022-US,1489,09/01/1998\|PZ-0051-P,1489,12/12/1997 |
| 233 | rat00145437 | 700524023 | ZS:217274.1 | RABMNOT01 | PZ-0022-US,974,09/01/1998\|PZ-0051-P,974,12/12/1997 |
| 234 | hu01349846 | 6112575 | LG:232035.3 | MCLDTXT03 | |
| 235 | hu01009468 | 6385123 | LG:232035.3 | FIBRUNT02 | |
| 236 | hu01160420 | 6424123 | LG:232035.3 | BRSTUNT01 | |
| 237 | hu01299909 | 6396655 | LG:232035.3 | UTRENOT10 | |
| 238 | hu01246478 | 6178515 | LG:232035.3 | BMARUNT02 | |
| 239 | hu01285278 | 4440191 | LG:232035.3 | SINTNOT22 | |
| 240 | hu01264445 | 6386840 | LG:232035.3 | FIBRUNT02 | |
| 241 | hu00198187 | 3438090 | IB:244681.1 | PENCNOT05 | PD-0389-P,1908,07/02/1997\|PD-0389-US,1908,06/24/1998 |
| 242 | hu00350676 | 2103752 | IB:401992.4 | BRAITUT02 | PD-0111-1-US,6437,12/05/1997\|PD-0275-P,6897,12/06/1996 |
| 243 | hu00837746 | 2134070 | IB:198531.2 | ENDCNOT01 | PD-0257-P,538,10/23/1996\|PD-0257-US,538,10/22/1997 |
| 244 | hu00818732 | 487437 | IB:198531.2 | HNT2AGT01 | |
| 245 | hu00171330 | 2215250 | IB:198531.2 | SINTFET03 | PD-0268-P,3146,10/31/1996\|PD-0268-US,3146,10/30/1997 |
| 246 | hu00471838 | 345407 | IB:198531.2 | THYMNOT02 | PD-0041-P,837,09/26/1995\|PD-0077-P,837,10/02/1995\|PD-0077- |
| | | | | | US,837,10/02/1996 |
| 247 | hu00496995 | 3244082 | IB:198531.2 | BRAINOT19 | PD-0370-P,944,05/29/1997\|PD-0370-US,944,05/12/1998 |
| 248 | hu00645541 | 2827143 | IB:214288.5 | TLYMNOT03 | PD-0363-P,100,05/02/1997\|PD-0363-US,100,04/30/1998 |
| 249 | hu00696057 | 2821267 | IB:214288.5 | ADRETUT06 | PD-0333-P,186,03/14/1997\|PD-0333-US,186,03/12/1998 |
| 250 | hu00792990 | 1742936 | IB:198531.3 | HIPONON01 | PD-0224-P,1823,08/28/1996\|PD-0224-US,1823,08/26/1997 |
| 251 | hu00549929 | 3335240 | IB:198531.3 | BRAIFET01 | PD-0369-P,1996,05/28/1997 |
| 252 | hu00897882 | 2747156 | IB:198531.3 | LUNGTUT11 | PD-0326-US,2414,03/04/1997\|PD-0326-P,847,03/07/1997 |
| 253 | hu00016152 | 1355979 | IB:198531.3 | LUNGNOT09 | PD-0177-P,455,05/15/1996\|PD-0177-US,455,05/14/1997 |
| 254 | hu00103257 | 3187872 | IB:198531.3 | THYMNON04 | PD-0381-P,920,06/02/1997\|PD-0381-US,920,06/02/1998 |
| 255 | hu00123714 | 1369013 | IB:198531.3 | SCORNON02 | PD-0161-P,1613,04/29/1996\|PD-0161-US,1613,04/29/1997 |
| 256 | hu00552910 | 3565618 | IB:198531.3 | BRONNOT02 | PS-0003-P,536,06/06/1997\|PS-0003-US,536,06/06/1997 |
| 257 | hu00855123 | 2953422 | IB:198531.3 | KIDNFET01 | PD-0339-P,2298,03/14/1997\|PD-0339-US,2298,03/12/1998 |
| 258 | hu00854305 | 2331423 | IB:198531.3 | COLNNOT11 | PD-0289-P,1724,12/18/1996\|PD-0289-US,1724,12/18/1997 |
| 259 | hu00397024 | 2837656 | IB:198531.3 | DRGLNOT01 | PD-0337-P,166,03/18/1997\|PD-0337-US,166,03/05/1998 |
| 260 | hu00143648 | 26403 | IB:198531.3 | SPLNFET01 | PD-0004-1-US,450,05/05/1995\|PD-0004-US,815,02/02/1994 |
| 261 | hu00705662 | 2235510 | IB:198531.3 | PANCTUT02 | PD-0191-US,2654,06/19/1997\|PD-0295-P,496,12/20/1996 |
| 262 | hu00593090 | 1213194 | IB:198531.3 | BRSTTUT01 | PD-0157-P,2341,04/26/1996\|PD-0157-US,2341,04/25/1997 |
| 263 | hu00459622 | 2716655 | IB:198531.3 | THYRNOT09 | PD-0331-P,842,02/13/1997\|PD-0331-US,842,02/10/1998 |
| 264 | hu00417548 | 1521103 | IB:198531.3 | BLADTUT04 | PD-0203-P,2555,08/01/1996\|PD-0203-US,2555,08/01/1997 |
| 265 | hu00076844 | 1558940 | IB:198531.3 | SPLNNOT04 | PD-0190-US,1794,06/19/1997\|PD-0215-P,214,07/30/1996 |
| 266 | hu00758235 | 1281986 | IB:198531.3 | COLNNOT16 | PD-0148-P,199,04/25/1996\|PD-0148-US,199,04/25/1997 |
| 267 | hu00339267 | 2936440 | IB:198531.3 | THYMFET02 | PD-0361-P,427,04/24/1997\|PD-0361-US,427,04/23/1998 |
| 268 | hu00166935 | 2360924 | IB:198531.3 | LUNGFET05 | PD-0281-P,1072,12/04/1996 |
| 269 | hu00942197 | 721037 | IB:198531.3 | SYNOOAT01 | PD-0099-US,2622,12/19/1996\|PD-0101-P,744,01/04/1996 |
| 270 | hu00525849 | 1282145 | IB:198531.3 | COLNNOT16 | PD-0148-P,334,04/25/1996\|PD-0148-US,334,04/25/1997 |
| 271 | hu00668292 | 2045951 | IB:198531.3 | THP1T7T01 | PD-0273-P,1525,10/31/1996\|PD-0273-US,698,10/28/1997 |
| 272 | hu00096927 | 1606444 | IB:198531.3 | LUNGNOT15 | PD-0208-P,2508,07/24/1996\|PD-0208-US,2508,07/23/1997 |
| 273 | hu00868174 | 2285437 | IB:198531.3 | BRAINON01 | PD-0274-P,800,11/20/1996 |
| 274 | hu00001500 | 1512742 | IB:214288.6 | PANCTUT01 | PD-0196-P,944,06/21/1996 |
| 275 | hu00598479 | 974641 | IB:214288.6 | MUSCNOT02 | PD-0131-P,1013,03/19/1996\|PD-0131-US,1013,03/18/1997 |
| 276 | hu00576699 | 1981621 | IB:214288.6 | LUNGTUT03 | PD-0248-P,1017,09/27/1996\|PD-0189-US,2315,06/19/1997 |
| 277 | hu00115451 | 547710 | IB:214288.6 | BEPINOT01 | PD-0056-P,373,10/31/1995\|PD-0056-US,373,10/16/1996 |
| 278 | hu00063441 | 3452386 | IB:214288.6 | UTRSNON03 | PD-0397-P,2648,07/02/1997\|PD-0397-US,2648,06/30/1998 |
| 279 | hu00117073 | 1541686 | IB:214288.6 | SINTTUT01 | PD-0210-US,1254,07/23/1997 |
| 280 | hu00034373 | 2508157 | IB:214288.6 | CONUTUT01 | PD-0300-P,2388,12/20/1996\|PD-0300-US,2388,12/18/1997 |
| 281 | hu00321357 | 1336918 | IB:214288.6 | COLNNOT13 | PD-0173-P,1168,05/16/1996\|PD-0173-US,1168,05/09/1997 |
| 282 | hu00606532 | 1524878 | IB:214288.6 | UCMCL5T01 | PD-0194-P,169,06/28/1996\|PD-0194-US,169,06/27/1997 |
| 283 | hu00331079 | 1003042 | IB:214288.6 | BRSTNOT03 | PD-0149-P,1064,04/12/1996\|PD-0149-US,1064,04/11/1997 |
| 284 | hu00091149 | 161598 | IB:214288.6 | ADENINB01 | PD-0033-1-US,1104,07/13/1995 |
| 285 | hu00390348 | 28378 | IB:214288.6 | SPLNFET01 | PD-0004-1-US,1181,05/05/1995 |
| 286 | hu00743094 | 2231816 | IB:214288.6 | PROSNOT16 | PD-0290-P,1497,12/19/1996\|PD-0290-US,1497,07/30/1997 |
| 287 | hu00652935 | 2638052 | IB:214288.6 | BONTNOT01 | PD-0302-P,959,01/27/1997\|PD-0302-US,959,01/23/1998 |
| 288 | hu00639806 | 2284174 | IB:214288.6 | BRAINON01 | PD-0274-P,160,11/20/1996 |
| 289 | hu00661686 | 64295 | IB:214288.6 | PLACNOB01 | PD-0011-US,1136,07/15/1994 |
| 290 | hu00698038 | 672180 | IB:214288.6 | CRBLNOT01 | PD-0089-P,1249,11/30/1995\|PD-0089-US,1249,11/27/1996 |
| 291 | hu00942589 | 1683714 | IB:214288.6 | PROSNOT15 | PD-0200-P,652,07/24/1996\|PD-0200-US,652,07/24/1997 |
| 292 | hu00689928 | 988257 | IB:214288.6 | LVENNOT03 | PD-0134-P,1374,03/21/1996\|PD-0134-US,1374,03/21/1997 |
| 293 | hu00201651 | 178582 | IB:214288.6 | PLACNOB01 | |
| 294 | hu00616103 | 957959 | IB:214288.6 | KIDNNOT05 | PD-0142-P,1538,04/10/1996\|PD-0142-US,1538,04/10/1997\|PD-0261 |
| | | | | | 1-US,3277,10/29/1997 |
| 295 | hu00300158 | 805000 | IB:214288.6 | BRAVTXT05 | PR-0005-P,1552,01/10/1996\|PR-0005-US,1552,01/10/1997 |
| 296 | hu00534530 | 2963787 | IB:214288.6 | SCORNOT04 | PD-0360-P,281,04/24/1997\|PD-0360-US,281,04/23/1998 |
| 297 | hu00924051 | 3255588 | IB:214288.6 | OVARTUN01 | PD-0374-P,1446,06/04/1997\|PD-0374-US,1446,05/18/1998 |
| 298 | hu00256307 | 268277 | IB:214288.6 | HNT2NOT01 | PD-0038-P,569,06/16/1995 |
| 299 | hu00114972 | 132527 | IB:214288.6 | BMARNOT02 | PD-0028-US,993,03/31/1995 |
| 300 | hu00330198 | 2351574 | IB:214288.6 | COLSUCT01 | PD-0278-P,889,12/05/1996\|PD-0278-US,889,11/21/1997 |
| 301 | hu00559222 | 1427071 | IB:214288.6 | SINTBST01 | PD-0209-P,1498,07/23/1996\|PD-0209-US,1498,07/22/1997 |
| 302 | hu00257435 | 1302726 | IB:214288.6 | PLACNOT02 | PD-0167-P,130,05/30/1996\|PD-0167-US,130,05/29/1997 |
| 303 | hu00641771 | 3014326 | IB:214288.6 | MUSCNOT07 | PD-0359-P,1688,04/11/1997\|PD-0359-US,1688,03/31/1998 |
| 304 | hu00157481 | 2793994 | IB:214288.6 | COLNTUT16 | PD-0322-P,2147,03/06/1997\|PD-0322-P,2147,03/06/1997\|PD-0322- |
| | | | | | US,2147,03/06/1998\|PD-0322-US,2147,03/06/1998 |
| 305 | hu00649531 | 3361302 | IB:214288.6 | PROSBPT02 | PD-0390-P,224,06/30/1997\|PD-0390-US,224,06/26/1998 |
| 306 | hu00256466 | 2228136 | IB:214288.6 | SEMVNOT01 | PD-0266-P,1344,10/28/1996\|PD-0266-US,1344,10/27/1997 |
| 307 | hu00637950 | 3439101 | IB:214288.6 | PENCNOT06 | PD-0389-P,2369,07/02/1997\|PD-0389-US,2369,06/24/1998 |
| 308 | hu00891318 | 1284989 | IB:214288.6 | COLNNOT16 | PD-0148-P,1259,04/25/1996\|PD-0148-US,1259,04/25/1997 |
| 309 | hu00267556 | 101709 | IB:214288.6 | ADRENOR01 | PD-0016-US,1581,10/14/1994 |
| 310 | hu00189229 | 2572961 | IB:214288.6 | HIPOAZT01 | PD-0324-P,1995,03/04/1997\|PD-0324-US,1995,02/26/1998 |
| 311 | hu00803857 | 1386853 | IB:214288.6 | CARGDIT02 | PR-0008-P,356,06/04/1996\|PR-0008-US,356,06/03/1997 |
| 312 | hu00428667 | 968283 | IB:214288.6 | BRSTNOT05 | PD-0133-P,3765,03/20/1996\|PD-0133-US,3765,03/20/1997 |
| 313 | hu00593649 | 2434871 | IB:214288.6 | BRAVUNT02 | PR-0012-P,1238,11/25/1996\|PR-0002-1-US,4272,11/19/1997 |
| 314 | hu00966556 | 529702 | IB:214288.6 | BRAINOT03 | PD-0058-P,156,10/25/1995\|PD-0088-US,156,11/27/1996\|PD-0092- |
| | | | | | P,156,12/13/1995 |
| 315 | hu00908022 | 691852 | IB:214288.6 | LUNGTUT02 | PD-0097-P,1114,12/20/1995\|PD-0097-US,1114,12/20/1996 |
| 316 | hu00247720 | 945009 | IB:214288.6 | RATRNOT02 | PD-0135-P,1422,03/20/1996\|PD-0135-US,1422,03/19/1997 |
| 317 | hu00093263 | 38327 | IB:214288.6 | HUVENOB01 | PD-0003-3-US,2045,08/26/1994\|PD-0003-1-US,4406,02/17/1994\|PD |
| | | | | | 0003-2-US,4766,04/28/1994 |
| 318 | hu00384521 | 2662208 | IB:214288.6 | ADRENOT08 | PD-0317-P,386,02/11/1997\|PD-0317-US,386,02/10/1998 |
| 319 | hu00322584 | 1236346 | IB:214288.6 | LUNGFET03 | PD-0146-P,1685,04/17/1996\|PD-0146-US,1685,04/16/1997 |
| 320 | hu00670453 | 1671467 | IB:214288.6 | BMARNOT03 | PD-0218-1-US,1474,03/12/1998\|PD-0218-P,1474,08/01/1996\|PD- |
| | | | | | 0218-US,1474,08/01/1997 |
| 321 | hu00056338 | 1450164 | IB:214288.6 | PLACNOT02 | PD-0193-P,1096,06/27/1996\|PD-0167-US,2456,05/29/1997 |
| 322 | hu00377154 | 1661168 | IB:214288.6 | BRSTNOT09 | PD-0219-P,1909,07/26/1996 |
| 323 | hu00289424 | 2675413 | IB:214288.6 | KIDNNOT19 | PD-0325-P,1547,03/05/1997 |
| 324 | hu00138864 | 2071881 | IB:214288.6 | ISLTNOT01 | PD-0259-P,987,10/28/1996\|PD-0259-US,987,10/28/1997 |
| 325 | hu00178785 | 41595 | IB:214288.6 | TBLYNOT01 | PD-0007-US,939,03/22/1994 |
| 326 | hu00886298 | 3567727 | IB:214288.6 | BRONNOT02 | PS-0003-P,1376,06/06/1997\|PS-0003-US,1376,06/16/1997 |
| 327 | hu00527500 | 438267 | IB:214288.6 | THYRNOT01 | PD-0049-P,1948,10/02/1995\|PD-0049-US,1948,09/30/1996 |
| 328 | hu00642045 | 1670227 | IB:214288.6 | BMARNOT03 | PD-0218-1-US,1070,03/12/1998\|PD-0218-P,1070,08/01/1996\|PD- |
| | | | | | 0218-US,1070,08/01/1997 |
| 329 | hu00093117 | 2733423 | IB:214288.6 | OVARTUT04 | P0-0313-P,1601,01/22/1997\|PD-0313-US,1601,01/22/1998 |
| 330 | hu00614740 | 237 | IB:214288.6 | U937NOT01 | PD-0001-3-US,181,02/14/1994\|PD-0001-US,181,07/17/1992\|PD- |
| | | | | | 0001-1-US,199,11/19/1992 |
| 331 | hu00265154 | 523746 | IB:214288.6 | MMLR2DT01 | PD-0044-P,2667,10/02/1995\|PD-0044-US,2667,10/02/1996 |
| 332 | hu00105683 | 1631414 | IB:214288.6 | COLNNOT19 | PD-0206-P,202,08/01/1996\|PD-0206-US,202,08/01/1997 |
| 333 | hu00117267 | 3327940 | IB:214288.6 | HEAONOT04 | PD-0387-P,2889,07/03/1997\|PD-0387-US,2889,06/30/1998 |
| 334 | hu00552139 | 436786 | IB:214288.6 | THYRNOT01 | PD-0049-P,1400,10/02/1995\|PD-0049-US,1400,09/30/1996 |
| 335 | hu00084843 | 3036523 | IB:214288.6 | PENCNOT02 | PD-0375-P,13,06/02/1997\|PD-0375-US,13,05/08/1998 |
| 336 | hu00633854 | 1724062 | IB:214288.6 | PROSNOT14 | PD-0201-P,135,07/29/1996\|PD-0201-US,135,07/28/1997\|PD-0210- |
| | | | | | P,135,07/24/1996 |
| 337 | hu00617818 | 1773452 | IB:214288.6 | MENTUNON3 | PD-0223-P,240,08/28/1996\|PD-0223-US,240,08/27/1997 |
| 338 | hu00168917 | 188 | IB:214288.6 | U937NOT01 | PD-0001-3-US,140,02/14/1994\|PD-0001-US,140,07/17/1992\|PD |
| | | | | | 0001-1-PCT,158,07/16/1993\|PD-0001-1-US,158,11/19/1992 |
| 339 | hu00897711 | 2728235 | IB:214288.6 | OVARTUT05 | PD-0313-P,3341,01/22/1997\|PD-0313-US,3341,01/22/1998 |
| 340 | hu00850688 | 2657709 | IB:214288.6 | LUNGTUT09 | PD-0310-P,2629,01/28/1997\|PD-0310-US,2629,01/28/1998 |
| 341 | hu00724100 | 66598 | IB:214288.6 | HUVESTB01 | PD-0003-2-US,6020,04/28/1994\|PD-0003-3-US,7136,08/26/1994 |
| 342 | hu00017402 | 2434736 | IB:214288.6 | BRAVUNT02 | PR-0012-P,1188,11/25/1996\|PR-0002-1-US,4222,11/19/19997 |
| 343 | hu00602053 | 2050370 | IB:214288.6 | LIVRFET02 | PD-0262-P,590,10/31/1996 |
| 344 | hu00521216 | 858026 | IB:214288.6 | NGANNOT01 | PD-0123-US,2374,02/28/1997\|PD-0125-P,2374,03/07/1996 |
| 345 | hu00403523 | 2922053 | IB:214288.6 | SININOT04 | PD-0344-P,403,03/17/1997\|PD-0344-US,403,03/16/1998 |
| 346 | hu00075141 | 1486724 | IB:214288.6 | UCMCL5T01 | PD-0184-P,1264,06/24/1996\|PD-0194-P,2587,06/28/1996\|PD-0194- |
| | | | | | US,2587,06/27/1997 |
| 347 | hu00022437 | 2319924 | IB:214288.6 | OVARNOT02 | PD-0293-P,1042,12/13/1996\|PD-0045-1-US,2869,11/10/1997 |
| 348 | hu00201683 | 2584108 | IB:214288.6 | BRAITUT22 | PD-0304-P,3129,01/28/1997\|PD-0304-US,3129,01/28/1998 |
| 349 | hu00860016 | 3318249 | IB:214288.6 | PROSBPT03 | PD-0390-P,2758,06/30/1997\|PD-0390-US,2758,06/26/1998 |
| 350 | hu00642216 | 1418795 | IB:214288.6 | KIDNNOT09 | PD-0186-P,317,06/25/1996\|PD-0186-US,317,06/24/1997 |
| 351 | hu00017647 | 1490935 | IB:214288.6 | UCMCL5T01 | PD-0184-P,1000,06/24/1996\|PD-0194-P,2324,06/28/1996\|PD-0194- |
| | | | | | US,2324,06/27/1997 |
| 352 | hu00336922 | 1972806 | IB:214288.6 | UCMCL5T01 | PD-0194-US,3439,06/27/1997\|PD-0272-P,838,10/23/1996 |
| 353 | hu00155892 | 857740 | IB:214288.6 | NGANNOT01 | PD-0123-US,2269,02/28/1997\|PD-0125-P,2269,03/07/1996 |
| 354 | hu00827284 | 777632 | IB:214288.6 | COLNNOT05 | PD-0105-P,1046,01/30/1996\|PD-0110-US,1046,01/30/1997 |
| 355 | hu00895708 | 1443789 | IB:214288.6 | THYRNOT03 | PD-0162-P,3595,05/29/1996\|PD-0162-US,3595,05/27/1997 |
| 356 | hu00219012 | 3360182 | IB:214288.6 | PROSTUT16 | PD-0391-P,3488,06/30/1997\|PD-0391-US,3488,06/19/1998 |
| 357 | hu00033057 | 1490047 | IB:214288.6 | UCMCL5T01 | PD-0194-P,2158,06/28/1996\|PD-0194-US,2158,06/27/1997\|PD-0184- |
| | | | | | P,834,06/24/1996 |
| 358 | hu00729121 | 168121 | IB:214288.6 | LIVRNOT01 | PD-0064-P,769,10/10/1995\|PD-0064-US,769,10/09/1996 |
| 359 | hu00935501 | 1753578 | IB:214288.6 | LIVRTUT01 | PD-0220-P,978,07/30/1996 |
| 360 | hu00644405 | 1705762 | IB:214288.6 | DUODNOT02 | PD-0207-P,2581,07/29/1996\|PD-0207-US,2581,07/29/1997 |
| 361 | hu00672261 | 947412 | IB:214288.6 | RATRNOT02 | PD-0135-P,1982,03/20/1996\|PD-0135-US,1982,03/19/1997 |
| 362 | hu00681415 | 2769452 | IB:214288.6 | COLANOT02 | PD-0321-P,695,02/12/1997\|PD-0321-P,695,02/12/1997\|PD-0321- |
| | | | | | US,695,02/10/1998\|PD-0321-US,695,02/10/1998 |
| 363 | hu01353645 | 59936 | IB:214288.6 | LUNGNOT01 | PD-0010-US,200,07/01/1994 |
| 364 | hu00155187 | 975960 | IB:214288.6 | MUSCNOT02 | PD-0131-P,1267,03/19/1996\|PD-0131-US,1267,03/18/1997 |
| 365 | hu00671536 | 436218 | IB:214288.6 | THYRNOT01 | PD-0049-P,1188,10/02/1995\|PD-0049-US,1188,09/30/1996 |
| 366 | hu00664723 | 2912496 | IB:214288.6 | KIDNTUT15 | |
| 367 | hu00874109 | 1833183 | IB:214288.6 | BRAINON01 | PD-0222-P,598,08/23/1996 |
| 368 | hu00515388 | 3323085 | IB:214288.6 | PTHYNOT03 | PD-0392-P,1103,07/01/1997\|PD-0392-US,1103,06/26/1998 |
| 369 | hu00081957 | 2595534 | IB:214288.6 | OVARTUT02 | PD-0312-P,733,01/21/1997\|PD-0312-US,733,01/21/1998 |
| 370 | hu00688447 | 158522 | IB:214288.6 | ADENINB01 | PD-0033-1-US,129,07/13/1995 |
| 371 | hu00120308 | 2739831 | IB:214288.6 | BRSTTUT14 | PD-0320-P,2223,02/13/1997\|PD-0320-US,2223,02/11/1998 |
| 372 | hu00700244 | 702877 | IB:214288.6 | SYNORAT03 | PD-0096-P,2526,12/15/1995\|PD-0096-US,2526,12/15/1995 |
| 373 | hu00641024 | 2779245 | IB:214288.6 | OVARTUT03 | PD-0329-P,964,03/07/1997\|PD-0329-US,964,03/05/1998 |
| 374 | hu00445320 | 3687255 | IB:214288.6 | HEAANOT01 | PD-0351-US,3136,04/23/1998 |
| 375 | hu00850230 | 1393468 | IB:214288.6 | THYRNOT03 | PD-0162-P,789,05/29/1996\|PD-0162-US,789,05/27/1997 |
| 376 | hu00381730 | 870700 | IB:214288.6 | LUNGAST01 | PD-0120-US,2959,02/28/1997\|PD-0120-P,734,02/29/1996 |
| 377 | hu00229540 | 1629121 | IB:214288.6 | COLNPOT01 | PD-0206-P,2816,08/01/1996\|PD-0206-US,2816,08/01/1997 |
| 378 | hu00439241 | 2616289 | IB:214288.6 | GBLANOT01 | PD-0338-P,1023,03/14/1997\|PD-0338-US,1023,03/12/1998 |
| 379 | hu00944105 | 1225832 | IB:214288.6 | COLNNOT01 | PD-0154-P,85,04/19/1996\|PD-0154-US,85,04/17/1997 |
| 380 | hu00107493 | 36001 | IB:214288.6 | HUVENOB01 | PD-0003-1-US,3141,02/17/1994\|PD-0003-2-US,3314,04/28/1994\|PD |
| | | | | | 0003-3-US,593,08/26/1994 |
| 381 | hu00580182 | 541261 | IB:214288.6 | | PD-0060-P,1209,10/30/1995\|PD-0131-P,1249,03/19/1996\|PD-0131- |
| | | | | | US,1249,03/18/1997 |
| 382 | hu00682152 | 986764 | IB:214288.6 | LVENNOT03 | PD-0134-P,968,03/21/1996\|PD-0134-US,968,03/21/1997 |
| 383 | hu00760642 | 3137305 | IB:214288.6 | SMCCNOT01 | PD-0377-P,1191,06/06/1997\|PD-0377-US,1191,05/20/1998 |
| 384 | hu00742742 | 1332445 | IB:214288.6 | PANCNOT07 | PD-0172-P,1235,05/23/1996\|PD-0172-US,1235,05/22/1997 |
| 385 | hu00082296 | 1233856 | IB:214288.6 | LUNGFET03 | PD-0146-P,638,04/17/1996\|PD-0146-US,638,04/16/1997 |
| 386 | hu00647803 | 2232952 | IB:214288.6 | PROSNOT16 | PD-0290-P,2027,12/19/1996\|PD-0290-US,2027,07/30/1997 |
| 387 | hu00098155 | 838885 | IB:214288.6 | PROSTUT05 | PD-0119-P,1652,02/27/1996\|PD-0119-US,1652,02/27/1997\|PD-0119- |
| | | | | | 1-US,186,09/30/1997 |
| 388 | hu00540983 | 830015 | IB:214288.6 | PROSTUT04 | PD-0117-P,2007,02/26/1996\|PD-0117-US,2007,02/26/1997\|PD-0197- |
| | | | | | US,431,06/23/1997 |
| 389 | hu00823826 | 968185 | IB:214288.6 | BRSTNOT05 | PD-0133-P,3720,03/20/1996\|PD-0133-US,3720,03/20/1997 |
| 390 | hu00316469 | 2266925 | IB:214288.6 | UTRSNOT02 | PD-0288-P,553,12/18/1996\|PD-0288-US,553,12/16/1997 |
| 391 | hu00186680 | 2232714 | IB:214288.6 | PROSNOT16 | PD-0290-P,1931,12/19/1996\|PD-0290-US,1931,07/30/1997 |
| 392 | hu00192684 | 1618829 | IB:214288.6 | BRAITUT12 | PD-0204-P,3326,07/26/1996 |
| 393 | hu00509719 | 862083 | IB:214288.6 | BRAITUT03 | PD-0126-1-US,1313,11/25/1997\|PD-0126-P,1313,03/08/1996\|PD- |
| | | | | | 0126-US,1313,03/07/1997 |
| 394 | hu00690657 | 567832 | IB:214288.6 | MMLR3DT01 | PD-0057-P,351,10/26/1995\|PD-0057-US,351,10/25/1996 |
| 395 | hu00319777 | 1737115 | IB:214288.6 | COLNNOT22 | PD-0226-P,1606,08/22/1997\|PD-0226-US,2195,08/22/1997 |
| 396 | hu00259981 | 1565719 | IB:214288.6 | HEALDIT02 | PR-0007-P,669,06/26/1996\|PR-0007-US,669,06/25/1997 |
| 397 | hu00555399 | 3319527 | IB:214288.6 | PROSBPT03 | PD-0390-P,3260,06/30/1997\|PD-0390-US,3260,06/26/1998 |
| 398 | hu00312115 | 1860508 | IB:214288.6 | PROSNOT18 | PD-0233-P,1766,09/05/1996\|PD-0233-US,1766,09/05/1997 |
| 399 | hu00663211 | 1674224 | IB:214288.6 | BLADNOT05 | PD-0203-P,816,08/01/1996\|PD-0203-US,816,08/01/1997 |
| 400 | hu00322633 | 909282 | IB:214288.6 | COLNNOT09 | PD-0129-P,751,03/19/1996\|PD-0129-US,751,03/18/1997 |
| 401 | hu00262441 | 2649949 | IB:214288.6 | THYMFET02 | PD-0361-P,2351,04/24/1997\|PD-0361-US,2351,04/23/1998 |
| 402 | hu00441056 | 2938018 | IB:214288.6 | THYMFET02 | PD-0361-P,1167,04/24/1997\|PD-0361-US,1167,04/23/1998 |
| 403 | hu00284223 | 2218628 | IB:214288.6 | LUNGNOT18 | PD-0263-P,116,10/28/1996\|PD-0263-US,116,10/28/1996 |
| 404 | hu00103469 | 2706495 | IB:214288.6 | PONSAZT01 | PD-0342-P,1054,03/24/1997\|PD-0342-US,1054,03/17/1998 |
| 405 | hu00133746 | 105473 | IB:214288.6 | BMARNOT02 | PD-0028-US,653,03/31/1995 |
| 406 | hu00224944 | 791482 | IB:214288.6 | PROSTUT03 | PD-0107-P,905,01/29/1996\|PD-0107-US,905,01/29/1997 |
| 407 | hu00785500 | 3038523 | IB:214288.6 | BRSTNOT16 | PD-0350-P,538,04/16/1997\|PD-0350-US,538,04/09/1998 |
| 408 | hu00252881 | 754354 | IB:214288.6 | BRAITUT02 | PD-0106-P,224,01/31/1996\|PD-0106-US,224,01/31/1997\|PD-0111-1- |
| | | | | | US,224,12/05/1997 |
| 409 | hu00458208 | 2588364 | IB:214288.6 | BRAITUT22 | PD-0304-P,4797,01/28/1997\|PD-0304-US,4797,01/28/1998 |
| 410 | hu00298622 | 2467836 | IB:214288.6 | THYRNOT08 | PD-0394-P,2791,07/02/1997\|PD-0394-US,2791,06/24/1998 |
| 411 | hu00484049 | 1807159 | IB:214288.6 | SINTNOT13 | PD-0234-P,1244,08/28/1996 |
| 412 | hu00911883 | 483414 | IB:214288.6 | HNT2RAT01 | PD-0075-P,409,10/03/1995\|PD-0075-US,409,10/03/1996 |
| 413 | hu00591366 | 1878392 | IB:214288.6 | LEUKNOT03 | PD-0246-P,2539,10/04/1996\|PD-0246-US,2539,10/02/1997 |
| 414 | hu00189607 | 3122394 | IB:214288.6 | LNODNOT05 | PD-0356-P,750,04/29/1997\|PD-0356-US,750,04/24/1998 |
| 415 | hu00901849 | 1632586 | IB:214288.6 | COLNNOT19 | PD-0206-P,382,08/01/1996\|PD-0206-US,382,08/01/1997 |
| 416 | hu00576852 | 1754034 | IB:214288.6 | LIVRTUT01 | PD-0220-P,1130,07/30/1996 |
| 417 | hu00262689 | 2615938 | IB:214288.6 | GBLANOT01 | PD-0338-P,873,03/14/1997\|PD-0338-US,873,03/12/1998 |
| 418 | hu00194559 | 776401 | IB:214288.6 | COLNNOT05 | PD-0105-P,690,01/30/1996\|PD-0110-US,690,01/30/1997 |
| 419 | hu00095974 | 3253386 | IB:214288.6 | OVARTUN01 | PD-0374-P,278,06/04/1997\|PD-0374-US,278,05/18/1998 |
| 420 | hu00399290 | 34572 | IB:766219.1 | THP1NOB01 | PD-0001-3-US,7660,02/14/1994\|PD-0001-4-US,7830,07/28/1994 |
| 421 | hu01329355 | 5613187 | LG:411605.7 | MONOTXN05 | |
| 422 | hu00529652 | 198957 | IB:758551.1 | KIDNNOT02 | PD-0024-US,2062,02/27/1995 |
| 423 | hu01054127 | 5613859 | LG:140649.1 | FIBRTXS07 | |
| 424 | hu00367261 | 2241085 | IB:075659.1 | PANCTUT02 | PD-0295-P,2198,12/20/1996\|PD-0191-US,4356,06/19/1997 |
| 425 | hu01216944 | 6304773 | LG:902506.4 | NERDTDN03 | |
| 426 | hu01169541 | 5949169 | LG:902506.4 | LIVRTUN04 | |
| 427 | hu00968022 | 5876719 | LG:902506.4 | BRAUNOT01 | |
| 428 | hu01042903 | 6507116 | LG:902506.4 | BRAHNOT02 | |
| 429 | hu01273836 | 6345009 | LG:902506.4 | LUNGDIS03 | |
| 430 | hu01270499 | 6345041 | LG:902506.4 | LUNGDIS03 | |
| 431 | hu01033738 | 6120407 | LG:902506.4 | BRAHNON05 | |
| 432 | hu01201032 | 6265395 | LG:902506.4 | MCLDTXN03 | |
| 433 | hu00524021 | 2781758 | IB:401992.9 | OVARTUT03 | PD-0329-P,2100,03/07/1997\|PD-0329-US,2100,03/05/1998 |
| 434 | hu00364988 | 1701981 | IB:401992.9 | BLADTUT05 | PD-0221-US,1609,07/31/1997\|PD-0202-P,2016,07/31/1996 |
| 435 | hu00932363 | 759186 | IB:401992.9 | BRAITUT02 | PD-0111-1-US,2855,12/05/1997\|PD-0111-P,673,02/22/1996\|PD- |
| | | | | | 0111-US,673,02/21/1997 |
| 436 | hu01103446 | 5657662 | LG:334807.2 | BSCNNOT03 | |
| 437 | hu01203049 | 3787443 | LG:334807.2 | BRAHNOT05 | |
| 438 | hu01068510 | 3788702 | LG:334807.2 | BRAHNOT05 | |
| 439 | hu01213943 | 4668706 | LG:334807.2 | SINTNOT24 | |
| 440 | hu01044863 | 6056729 | LG:334807.2 | BRAENOT04 | |
| 441 | hu01200968 | 3762849 | LG:334807.2 | BRAHDIT03 | |
| 442 | hu01018101 | 6558417 | LG:334807.2 | BRAFNON02 | |
| 443 | hu01329733 | 6559956 | LG:334807.2 | BRAFNON02 | |
| 444 | hu01337095 | 6210954 | LG:334807.2 | ARTANOT06 | |
| 445 | hu01282087 | 5808476 | LG:334807.2 | BRAHNOT02 | |
| 446 | hu01090767 | 6555152 | LG:334807.2 | BRAFNON02 | |
| 447 | hu00979175 | 5888375 | LG:411449.4 | LIVRNON08 | |
| 448 | hu01182918 | 5880566 | LG:411449.4 | LIVRNON08 | |
| 449 | hu01343318 | 5884946 | LG:411449.4 | LIVRNON08 | |
| 450 | hu01147865 | 5889095 | LG:411449.4 | LIVRNON08 | |
| 451 | hu01074751 | 5880061 | LG:411449.4 | LIVRNON08 | |
| 452 | hu01296205 | 5890510 | LG:411449.4 | LIVRNON08 | |
| 453 | hu00970451 | 5885109 | LG:411449.4 | LIVRNON08 | |
| 454 | hu01325064 | 6129550 | LG:334807.3 | BRAHNON05 | |
| 455 | hu01065608 | 6127983 | LG:334807.3 | BRAHNON05 | |
| 456 | hu01046405 | 6111849 | LG:411449.5 | MCLDTXT03 | |
| 457 | hu01285201 | 5695240 | LG:411449.6 | BRSTTUT13 | |
| 458 | hu01013366 | 5815925 | LG:411449.6 | PROSTUS23 | |
| 459 | hu01326433 | 6138342 | LG:411449.6 | BMARTXT03 | |
| 460 | hu01147225 | 6522432 | LG:411449.6 | CONFTDT02 | |
| 461 | hu01344254 | 6310665 | LG:411449.6 | NERDTDN03 | |
| 462 | hu01128192 | 6129270 | LG:411449.6 | BRAHNON05 | |
| 463 | hu01067191 | 6408546 | LG:411449.6 | UTREDIT10 | |
| 464 | hu01070449 | 6305061 | LG:411449.6 | NERDTDN03 | |
| 465 | hu01324078 | 6588841 | LG:411449.6 | TLYMUNT03 | |
| 466 | hu01051012 | 6552781 | LG:411449.6 | BRAFNON02 | |
| 467 | hu01324557 | 6482476 | LG:411449.6 | PROSTMC01 | |
| 468 | hu01070929 | 6343922 | LG:411449.6 | LUNGDIS03 | |
| 469 | hu00983044 | 5919979 | LG:411449.6 | BRAIFET02 | |
| 470 | hu01170686 | 3783111 | LG:411449.6 | BRAHDIT04 | |
| 471 | hu00968530 | 4518248 | LG:411449.6 | SINJNOT03 | |
| 472 | hu00010006 | 2492677 | IB:475199.2 | ADRETUT05 | PD-0318-P,253,02/11/1997\|PD-0318-US,253,02/09/1998 |
| 473 | hu00957158 | 3357454 | IB:796437.1 | PROSTUT16 | PD-0391-P,2579,06/30/1997\|PD-0391-US,2579,06/19/1998 |
| 474 | hu01264231 | 6126885 | LG:334807.5 | BRAHNON05 | |
| 475 | hu00305008 | 188581 | IB:475199.3 | CARDNOT01 | PD-0019-US,2898,01/05/1995 |
| 476 | hu00631844 | 2135063 | IB:475199.3 | ENDCNOT01 | PD-0257-P,918,10/23/1996\|PD-0257-US,918,10/22/1997 |
| 477 | hu00667379 | 1331867 | IB:475199.4 | PANCNOT07 | PD-0172-P,1082,05/23/1996\|PD-0172-US,1082,05/22/1997 |
| 478 | hu00200113 | 3960113 | IB:475199.4 | HEARFET02 | PD-0353-US,3090,03/17/1998 |
| 479 | hu00657975 | 1644841 | IB:475199.4 | HEARFET01 | PD-0212-P,743,07/26/1996 |
| 480 | hu00090130 | 2081894 | IB:475199.4 | UTRSNOT08 | PD-0287-P,908,12/04/1996\|PD-0287-US,908,11/19/1997 |
| 481 | hu00731030 | 3069069 | IB:475199.4 | UTRSNOR01 | PD-0365-P,535,04/18/1997\|PD-0365-US,535,04/16/1998 |
| 482 | hu00112740 | 4323184 | IB:475199.4 | TLYMUNT01 | PS-0009-P,341,04/16/1998\|PS-0009-US,341,04/16/1999 |
| 483 | hu00405180 | 2539031 | IB:475199.4 | BONRTUT01 | PD-0303-P,552,01/27/1997 |
| 484 | hu00801145 | 742282 | IB:475199.4 | PANCNOT04 | PD-0214-US,1511,08/01/1997\|PD-0118-P,521,02/26/1996\|PD-0118- |
| | | | | | US,521,02/26/1997 |
| 485 | hu00710542 | 2028355 | IB:475199.4 | KERANOT02 | PD-0260-P,1161,10/25/1996\|PD-0116-1-US,2583,10/24/1997 |
| 486 | hu00141175 | 159425 | IB:475199.4 | ADENINB01 | PD-0033-US,353,03/29/1995\|PD-0033-1-US,436,07/13/1995 |
| 487 | hu00414290 | 671593 | IB:475199.4 | CRBLNOT01 | PD-0089-P,1069,11/30/1995\|PD-0089-US,1069,11/27/1996 |
| 488 | hu00264315 | 2604050 | IB:475199.4 | LUNGTUT07 | PD-0310-P,253,01/28/1997\|PD-0310-US,253,01/28/1998 |
| 489 | hu00049062 | 3685138 | IB:475199.4 | HEAANOT01 | PD-0351-US,2445,04/23/1998 |
| 490 | hu00059621 | 3957184 | IB:475199.4 | HEARFET02 | PD-0353-US,2230,03/17/1998 |
| 491 | hu00166903 | 2908269 | IB:475199.4 | THYMNOT05 | PD-0393-P,1702,07/02/1997\|PD-0393-US,1702,06/24/1998 |
| 492 | hu00503917 | 942890 | IB:475199.4 | ADRENOT03 | PD-0144-P,863,04/10/1996\|PD-0144-US,863,04/10/1997 |
| 493 | hu00134926 | 1332741 | IB:475199.4 | PANCNOT07 | PD-0172-P,1307,05/23/1996\|PD-0172-US,1307,05/22/1997 |
| 494 | hu00445742 | 1316096 | IB:475199.4 | BLADTUT02 | PD-0171-P,807,05/23/1996\|PD-0171-US,807,05/22/1997 |
| 495 | hu00336087 | 289538 | IB:475199.4 | TMLR3DT01 | PD-0037-US,146,05/15/1995 |
| 496 | hu00929610 | 679064 | IB:475199.4 | UTRSNOT02 | PD-0095-P,273,12/29/1995\|PD-0095-US,273,12/23/1996 |
| 497 | hu00079146 | 2439313 | IB:475199.4 | EOSITXT01 | PR-0011-US,2352,08/29/1997\|PR-0013-P,996,11/25/1996 |
| 498 | hu00273268 | 909094 | IB:475199.4 | COLNNOT09 | PD-0129-P,675,03/19/1996\|PD-0129-US,675,03/18/1997 |
| 499 | hu00628912 | 2670325 | IB:475199.4 | ESOGTUT02 | PD-0323-P,1390,03/05/1997\|PD-0323-US,1390,03/02/1998 |
| 500 | hu00086716 | 2443220 | IB:475199.4 | EOSITXT01 | PR-0013-P,2430,11/25/1996\|PR-0011-US,3786,08/29/1997 |
| 501 | hu00614476 | 2149587 | IB:475199.4 | BRAINOT09 | PD-0163-US,2432,05/20/1997\|PD-0275-P,398,12/06/1996 |
| 502 | hu00148645 | 291706 | IB:475199.4 | TMLR3DT01 | PD-0037-US,757,05/15/1995 |
| 503 | hu00833681 | 2665513 | IB:475199.4 | ADRENOT08 | PD-0317-P,1706,02/11/1997\|PD-0317-US,1706,02/10/1998 |
| 504 | hu00532388 | 2953235 | IB:475199.4 | KIDNFET01 | PD-0339-P,2225,03/14/1997\|PD-0339-US,2225,03/12/1998 |
| 505 | hu00436439 | 890040 | IB:475199.4 | STOMTUT01 | PD-0127-P,435,03/08/1996 |
| 506 | hu00088932 | 1609748 | IB:475199.4 | COLNTUT06 | PD-0206-P,3127,08/01/1996\|PD-0206-US,3127,08/01/1997 |
| 507 | hu00149178 | 2293179 | IB:475199.4 | BRAINON01 | PD-0274-P,5202,11/20/1996 |
| 508 | hu00426594 | 2766633 | IB:475199.4 | BRSTNOT12 | PD-0320-P,1866,02/13/1997\|PD-0320-US,1866,02/11/1998 |
| 509 | hu00065947 | 2736649 | IB:475199.4 | OVARNOT09 | PD-0311-P,782,01/27/1997\|PD-0311-US,782,01/23/1998 |
| 510 | hu00915854 | 2304562 | IB:475199.4 | BRSTNOT05 | PD-0292-P,4157,12/19/1996\|PD-0292-US,4157,12/18/1997 |
| 511 | hu00944169 | 2763026 | IB:475199.4 | BRSTNOT12 | PD-0320-P,346,02/13/1997\|PD-0320-US,346,02/11/1998 |
| 512 | hu00775518 | 1377278 | IB:475199.4 | LUNGNOT10 | PD-0179-P,663,05/13/1996\|PD-0179-US,663,05/09/1997 |
| 513 | hu00369357 | 1998146 | IB:475199.4 | BRSTTUT03 | PD-0241-P,1380,10/01/1997\|PD-0143-1-US,5410,10/01/1997 |
| 514 | hu00668950 | 2790194 | IB:475199.4 | COLNTUT16 | PD-0322-P,409,03/06/1997\|PD-0322-P,409,03/06/1997\|PD-0322- |
| | | | | | US,409,03/06/1998\|PD-0322-US,409,03/06/1998 |
| 515 | hu00891829 | 2530965 | IB:475199.4 | GBLANOT02 | PD-0306-P,1105,01/27/1997\|PD-0306-US,1105,01/23/1998 |
| 516 | hu00036080 | 2664895 | IB:475199.4 | ADRENOT08 | PD-0317-P,1477,02/11/1997\|PD-0317-US,1477,02/10/1998 |
| 517 | hu00904410 | 1836569 | IB:475199.4 | BRAINON01 | PD-0222-P,1620,08/23/1996 |
| 518 | hu00505381 | 1281254 | IB:475199.4 | COLNNOT16 | PD-0148-P,222,04/25/1996\|PD-0148-US,222,04/25/1997 |
| 519 | hu00431437 | 2658750 | IB:475199.4 | LUNGTUT09 | PD-0310-P,3084,01/28/1997\|PD-0310-US,3084,/01/28/1998 |
| 520 | hu00851911 | 1468402 | IB:475199.4 | PANCTUT02 | PD-0191-P,745,06/21/1996\|PD-0191-US,745,06/19/1997 |
| 521 | hu00044828 | 94971 | IB:475199.4 | PITUNOT01 | PD-0017-US,537,10/05/1994 |
| 522 | hu00562591 | 3202791 | IB:475199.4 | PENCNOT02 | PD-0375-P,1359,06/02/1997\|PD-0375-US,1359,05/08/1998 |
| 523 | hu00682264 | 1623750 | IB:475199.4 | BRAITUT13 | PD-0205-P,1504,07/26/1996\|PD-0205-US,1504,07/24/1997 |
| 524 | hu00505828 | 2269607 | IB:475199.4 | UTRSNOT02 | PD-0288-P,1600,12/18/1996\|PD-0288-US,1600,12/16/1997 |
| 525 | hu00881192 | 555662 | IB:475199.4 | SCORNOT01 | PD-0066-P,1310,10/31/1995 |
| 526 | hu00486165 | 3273425 | IB:475199.4 | PROSBPT06 | PD-0376-P,440,06/02/1997\|PD-0376-US,440,05/15/1998 |
| 527 | hu00741963 | 488117 | IB:475199.4 | HNT2AGT01 | |
| 528 | hu00424866 | 955963 | IB:475199.4 | KIDNNOT05 | PD-0261-1-US,2496,10/29/1997\|PD-0142-P,757,04/10/1996\|PD- |
| | | | | | 0142-US,757,04/10/1997 |
| 529 | hu00575940 | 837468 | IB:475199.4 | PROSNOT07 | PD-0119-P,1178,02/27/1996\|PD-0119-US,1178,02/27/1997\|PD-0119- |
| | | | | | 1-US,5198,09/30/1997 |
| 530 | hu00952636 | 2668611 | IB:475199.4 | ESOGTUT02 | PD-0323-P,703,03/05/1997\|PD-0323-US,703,03/02/1998 |
| 531 | hu00777181 | 641056 | IB:475199.4 | BRSTNOT03 | PD-0061-US,1306,11/15/1996\|PD-0065-P,1306,11/28/1995 |
| 532 | hu00820669 | 1624341 | IB:475199.4 | BRAITUT13 | PD-0205-P,1728,07/26/1996\|PD-0205-US,1728,07/24/1997 |
| 533 | hu00950657 | 2152009 | IB:475199.4 | BRAINOT09 | PD-0275-P,1126,12/06/1996\|PD-0163-US,3160,05/20/1997 |
| 534 | hu00492926 | 672412 | IB:475199.4 | CRBLNOT01 | PD-0089-P,1324,11/30/1995\|PD-0089-US,1324,11/27/1996 |
| 535 | hu00386691 | 68947 | IB:475199.4 | HUVESTB01 | PD-0003-2-US,6899,04/28/1994\|PD-0003-3-US,8015,08/26/1994 |
| 536 | hu00691935 | 1312213 | IB:475199.4 | COLNFET02 | PD-0168-P,1432,05/21/1996\|PD-0168-US,1432,05/21/1997 |
| 537 | hu00672864 | 738387 | IB:475199.4 | TONSNOT01 | PD-0100-P,1406,01/05/1996\|PD-0100-US,1406,01/03/1997 |
| 538 | hu00824866 | 1869727 | IB:475199.4 | SKINBIT01 | PD-0253-P,1085,10/03/1996\|PD-0253-US,1085,09/30/1997 |
| 539 | hu00849554 | 2834526 | IB:475199.4 | TLYMNOT03 | PD-0363-P,2923,05/02/1997\|PD-0363-US,2923,04/30/1998 |
| 540 | hu00694880 | 1547894 | IB:475199.4 | PROSNOT06 | PD-0199-P,3994,07/30/1996 |
| 541 | hu00488548 | 3566219 | IB:475199.4 | BRONNOT02 | PS-0003-P,791,06/06/1997\|PS-0003-US,791,06/06/1997 |
| 542 | hu00861598 | 114170 | IB:475199.4 | TESTNOT01 | |
| 543 | hu00748875 | 2264814 | IB:475199.4 | UTRSNOT02 | PD-0288-P,3377,12/18/1996\|PD-0288-US,3377,12/16/1997 |
| 544 | hu00430192 | 219339 | IB:475199.4 | STOMNOT01 | PD-0022-US,3085,02/07/1995 |
| 545 | hu00001649 | 733063 | IB:475199.4 | LUNGNOT03 | PD-0102-P,1089,01/18/1996 |
| 546 | hu00182128 | 3118194 | IB:475199.4 | LUNGTUT13 | PD-0358-P,2885,04/18/1997\|PD-0358-US,2885,04/17/1998 |
| 547 | hu00550066 | 2661257 | IB:475199.4 | ADRENOT08 | PD-0317-P,24,02/11/1997\|PD-0317-US,24,02/10/1998 |
| 548 | hu00496130 | 2990381 | IB:475199.4 | KIDNFET02 | PD-0355-P,512,04/11/1997\|PD-0355-US,512,03/30/1998 |
| 549 | hu00515714 | 548160 | IB:475199.4 | BEPINOT01 | PD-0056-P,504,10/31/1995\|PD-0056-US,504,10/16/1996 |
| 550 | hu00723751 | 1370295 | IB:475199.4 | BSTMNON02 | PD-0160-1-US,260,06/30/1998\|PD-0160-P,260,04/26/1996 |
| 551 | hu00150946 | 1887457 | IB:475199.4 | BLADTUT07 | PD-0238-P,1452,09/24/1996\|PD-0238-US,1452,09/23/1997 |
| 552 | hu00111878 | 2210818 | IB:475199.4 | SINTFET03 | PD-0268-P,1511,10/31/1996\|PD-0268-US,1511,10/30/1997 |
| 553 | hu00128465 | 3176964 | IB:475199.4 | UTRSTUT04 | PD-0383-P,1304,05/16/1997\|PD-0383-US,1304,05/08/1998 |
| 554 | hu00561474 | 4209737 | IB:475199.4 | BRONDIT01 | PS-0004-US,1039,12/13/1996\|PS-0006-P,90,12/23/1997 |
| 555 | hu00279622 | 687475 | IB:475199.4 | UTRSNOT02 | PD-0095-P,2480,12/29/1995\|PD-0095-US,2480,12/23/1996 |
| 556 | hu00895600 | 3461089 | IB:475199.4 | 293TF1T01 | PD-0385-P,1453,07/01/1997\|PD-0385-US,1453,06/30/1998 |
| 557 | hu00139834 | 158748 | IB:475199.4 | ADENINB01 | PD-0033-US,184,03/29/1995\|PD-0033-1-US,203,07/13/1995 |
| 558 | hu00872570 | 583799 | IB:475199.4 | PROSNOT02 | PD-0086-P,472,10/27/1995\|PD-0086-US,472,10/21/1996 |
| 559 | hu00624854 | 2784522 | IB:475199.4 | BRSTNOT13 | PD-0335-P,915,03/21/1997\|PD-0335-US,915,03/20/1998 |
| 560 | hu00571395 | 2441763 | IB:475199.4 | EOSITXT01 | PR-0013-P,1900,11/25/1996\|PR-0011-US,3256,08/29/1997 |
| 561 | hu00872139 | 741986 | IB:475199.4 | PANCNOT04 | PD-0214-US,1472,08/01/1997\|PD-0118-P,482,02/26/1996\|PD-0118- |
| | | | | | US,482,02/26/1997 |
| 562 | hu00498235 | 3336778 | IB:475199.4 | SPLNNOT10 | PD-0378-P,2152,06/05/1997\|PD-0378-US,2152,05/19/1998 |
| 563 | hu00736942 | 2631182 | IB:475199.4 | COLNTUT15 | PD-0305-P,519,01/22/1997\|PD-0305-US,519,01/22/1998 |
| 564 | hu00437945 | 630531 | IB:475199.4 | KIDNNOT05 | PD-0063-P,1037,11/22/1995\|PD-0261-1-US,1037,10/29/1997 |
| 565 | hu00677247 | 2371227 | IB:475199.4 | ADRENOT07 | PD-0301-P,2795,12/19/1996\|PD-0301-US,2795,12/17/1997 |
| 566 | hu00287089 | 1001180 | IB:475199.4 | BRSTNOT03 | PD-0149-P,458,04/12/1996\|PD-0149-US,458,04/11/1997 |
| 567 | hu00893466 | 270390 | IB:475199.4 | HNT2NOT01 | PD-0038-P,1247,06/16/1995 |
| 568 | hu00539398 | 46859 | IB:475199.4 | CORNNOT01 | PD-0006-1-US,624,06/07/1995\|PD-0006-US,877,03/31/1994 |
| 569 | hu00856774 | 630234 | IB:475199.4 | KIDNNOT05 | PD-0063-P,928,11/22/1995\|PD-0261-1-US,928,10/29/1997 |
| 570 | hu00692996 | 1991586 | IB:475199.4 | CORPNOT02 | PD-0185-US,4814,06/26/1997\|PD-0256-P,994,10/23/1996 |
| 571 | hu00966768 | 3925490 | IB:475199.4 | KIDNNOT19 | |
| 572 | hu00030404 | 3401566 | IB:475199.4 | ESOGNOT03 | PD-0386-P,92,07/01/1997\|PD-0386-US,92,06/24/1998 |
| 573 | hu00332510 | 3338452 | IB:475199.4 | SPLNNOT10 | PD-0378-P,2551,06/05/1997\|PD-0378-US,2551,05/19/1998 |
| 574 | hu00650103 | 1921936 | IB:475199.4 | BRSTTUT01 | PD-0240-P,2513,09/30/1996\|PD-0240-US,2513,09/29/1997 |
| 575 | hu00003951 | 2406413 | IB:475199.4 | BSTMNON02 | PD-0160-1-US,2648,06/30/1998 |
| 576 | hu00560152 | 3271032 | IB:475199.4 | BRAINOT20 | PD-0370-P,3973,05/29/1997\|PD-0370-US,3973,05/12/1998 |
| 577 | hu00825211 | 579510 | IB:475199.4 | BRAVTXT05 | PR-0003-US,1890,11/13/1996\|PR-0004-P,1890,12/15/1995 |
| 578 | hu00470440 | 1999081 | IB:475199.4 | BRSTTUT03 | PD-0241-P,1765,10/01/1997\|PD-0143-1-US,5795,10/01/1997 |
| 579 | hu00174710 | 2118363 | IB:475199.4 | BRSTTUT02 | PD-0255-P,1034,10/23/1996 |
| 580 | hu00049450 | 2650642 | IB:475199.4 | LUNGTUT13 | PD-0358-P,3765,04/18/1997\|PD-0358-US,3765,04/17/1998 |
| 581 | hu00516337 | 2766825 | IB:475199.4 | BRSTNOT12 | PD-0320-P,1954,02/13/1997\|PD-0320-US,1954,02/11/1998 |
| 582 | hu00779021 | 94970 | IB:475199.4 | PITUNOT01 | PD-0017-US,536,10/05/1994 |
| 583 | hu00870760 | 3173908 | IB:475199.4 | UTRSTUT04 | PD-0383-P,395,05/16/1997\|PD-0383-US,395,05/08/1998 |
| 584 | hu00507709 | 837155 | IB:475199.4 | PROSNOT07 | PD-0119-P,1089,02/27/1996\|PD-0119-US,1089,02/27/1997\|PD-0119 |
| | | | | | 1-US,5109,09/30/1997 |
| 585 | hu00826066 | 547537 | IB:475199.4 | BEPINOT01 | PD-0056-P,309,10/31/1995\|PD-0056-US,309,10/16/1996 |
| 586 | hu00526933 | 672497 | IB:475199.4 | CRBLNOT01 | PD-0089-P,1348,11/30/1995\|PD-0089-US,1348,11/27/1996 |
| 587 | hu00634709 | 2720987 | IB:475199.4 | LUNGTUT10 | PD-0327-P,579,03/05/1997\|PD-0327-US,579,03/03/1998 |
| 588 | hu00279274 | 1003869 | IB:475199.4 | BRSTNOT03 | PD-0149-P,1385,04/12/1996\|PD-0149-US,1385,04/11/1997 |
| 589 | hu00046789 | 1259346 | IB:475199.4 | MENITUT03 | PD-0147-P,1751,04/17/1996\|PD-0147-US,1751,04/17/1997 |
| 590 | hu00686725 | 2474822 | IB:475199.4 | SMCANOT01 | PD-0298-P,113,01/30/1998\|PD-0298-US,113,01/30/1998 |
| 591 | hu01211796 | 6120495 | LG:334807.7 | BRAHNON05 | |
| 592 | hu01277569 | 6030522 | LG:334807.7 | BRAHNON05 | |
| 593 | hu01227913 | 6127887 | LG:334807.7 | BRAHNON05 | |
| 594 | hu00019002 | 550609 | IB:230151.1 | BEPINOT01 | PD-0056-P,1262,10/31/1995\|PD-0056-US,1262,10/16/1996 |
| 595 | hu00074988 | 2834338 | IB:230151.1 | TLYMNOT03 | PD-0363-P,2867,05/02/1997\|PD-0363-US,2867,04/30/1998 |
| 596 | hu00682203 | 1209024 | IB:230151.1 | BRSTNOT02 | PD-0157-P,214,04/26/1996\|PD-0157-US,214,04/25/1997 |
| 597 | hu00405958 | 2160735 | IB:230151.1 | ENDCNOT02 | PD-0257-P,3672,10/23/1996\|PD-0257-US,3672,10/22/1997 |
| 598 | hu00902417 | 1006278 | IB:230151.1 | HEALDIT02 | PR-0006-P,950,03/26/1996\|PR-0006-US,950,03/26/1997 |
| 599 | hu00882139 | 2588088 | IB:230151.1 | BRAITUT22 | PD-0304-P,4695,01/28/1997\|PD-0304-US,4695,01/28/1998 |
| 600 | hu00444939 | 2160580 | IB:230151.1 | ENDCNOT02 | PD-0257-P,3592,10/23/1996\|PD-0257-US,3592,10/22/1997 |
| 601 | hu00996720 | 6523403 | LG:399851.3 | CONFTDT02 | |
| 602 | hu00979613 | 5953606 | LG:993791.1 | SKINTDT01 | |
| 603 | hu01217988 | 6372764 | LG:993791.1 | FIBAUNT02 | |
| 604 | hu01131267 | 6373435 | LG:993791.1 | FIBAUNT02 | |
| 605 | hu01057108 | 6387476 | LG:993791.1 | FIBRUNT02 | |
| 606 | hu01127156 | 6410750 | LG:993791.1 | UTREDIT10 | |
| 607 | hu01315257 | 6372052 | LG:993791.1 | FIBAUNT02 | |
| 608 | hu01127950 | 6305576 | LG:993791.1 | NERDTDN03 | |
| 609 | hu01277533 | 6122907 | LG:993791.1 | BRAHNON05 | |
| 610 | hu01313696 | 6422660 | LG:993791.1 | BRSTUNT01 | |
| 611 | hu01262379 | 6454929 | LG:993791.1 | COLNDIC01 | |
| 612 | hu01184935 | 6383359 | LG:993791.1 | FIBRUNT02 | |
| 613 | hu01309738 | 6422668 | LG:993791.1 | BRSTUNT01 | |
| 614 | hu01216150 | 6118137 | LG:993791.1 | BRAHNON05 | |
| 615 | hu01330055 | 6126413 | LG:993791.1 | BRAHNON05 | |
| 616 | hu01007235 | 6425148 | LG:993791.1 | BRSTUNT01 | |
| 617 | hu00627285 | 1857027 | IB:232035.1 | PROSNOT18 | PD-0233-P,473,09/05/1996\|PD-0233-US,473,09/05/1997 |
| 618 | rat00198924 | 700368142 | ZS:221520.3 | RAHYNOT01 | PZ-0033-P,374,10/08/1997\|PZ-0033-US,374,10/05/1998 |
| 619 | rat00147149 | 700494046 | ZS:221520.3 | RABCNOT01 | PZ-0042-P,1807,11/05/1997\|PZ-0012-US,3608,08/04/1998 |
| 620 | rat00122728 | 700369876 | ZS:221520.3 | RAHYNOT01 | PZ-0033-P,1222,10/08/1997\|PZ-0033-US,1222,10/05/1998 |
| 621 | rat00102041 | 700255036 | ZS:221520.3 | RABMNOT02 | PZ-0022-US,4154,09/01/1998\|PZ-0030-P,665,10/08/1997 |
| 622 | rat00244239 | 700494879 | ZS:221520.3 | RABCNOT01 | PZ-0042-P,2222,11/05/1997\|PZ-0012-US,4023,08/04/1998 |
| 623 | hu00860841 | 467072 | IB:232035.2 | LATRNOT01 | PD-0053-P,2338,10/10/1995\|PD-0053-US,2338,10/09/1996 |
| 624 | hu00329532 | 464308 | IB:232035.2 | LATRNOT01 | PD-0053-P,1524,10/10/1995\|PD-0053-US,1524,10/09/1996 |
| 625 | hu00268088 | 2798569 | IB:232035.3 | NPOLNOT01 | PD-0340-P,1582,03/28/1997\|PD-0340-US,1582,03/27/1998 |
| 626 | hu00754801 | 2230026 | IB:232035.3 | PROSNOT16 | PD-0290-P,639,12/19/1996\|PD-0290-US,639,07/30/1997 |
| 627 | rat00227438 | 700536442 | ZS:221520.5 | RAEONOT02 | PZ-0024-US,1652,09/10/1998\|PZ-0052-P,1652,12/11/1997 |
| 628 | rat00148580 | 701608605 | ZS:221520.5 | RALITXT09 | PZ-0116-P,1049,04/29/1999 |
| 629 | rat00148626 | 700815950 | ZS:221520.5 | RABFNOT03 | PZ-0013-US,1517,08/07/1998\|PZ-0063-P,424,02/10/1998 |
| 630 | rat00068841 | 700918115 | ZS:221520.5 | RAXSNOT01 | PZ-0100-P,1079,11/12/1998 |
| 631 | rat00126954 | 701257014 | ZS:221520.5 | RALITXT03 | PZ-0096-P,531,10/09/1998 |
| 632 | rat00170875 | 700461506 | ZS:221520.5 | RATHNOC02 | PZ-0049-P,75,11/07/1997\|PZ-0049-US,75,11/06/1998 |
| 633 | rat00059770 | 700023402 | ZS:221520.5 | RANPNOT01 | PZ-0008-P,1143,07/10/1997\|PZ-0008-US,1143,07/10/1998\|PZ-0009- |
| | | | | | P,1143,07/09/1997 |
| 634 | rat00159473 | 701346456 | ZS:221520.5 | RALITXT11 | PZ-0094-P,1790,09/11/1998 |
| 635 | rat00061995 | 700123372 | ZS:221520.5 | RABHNOT01 | PZ-0004-P,663,07/09/1997\|PZ-0004-US,663,07/09/1998 |
| 636 | rat00239188 | 700774145 | ZS:221520.5 | RABONOT01 | PZ-0077-P,1748,04/07/1998\|PZ-0076-US,4052,04/06/1999 |
| 637 | rat00226688 | 701608169 | ZS:221520.5 | RALITXT09 | PZ-0116-P,927,04/29/1999 |
| 638 | rat00079387 | 700322834 | ZS:221520.5 | RAHINOT01 | PZ-0025-US,3006,09/09/1998\|PZ-0025-P,393,09/12/1997 |
| 639 | rat00069857 | 701195154 | ZS:221520.5 | RALINOT02 | PZ-0026-US,1964,09/04/1998\|PZ-0082-P,634,05/01/1998 |
| 640 | rat00201032 | 701509245 | ZS:221520.5 | RALITXT02 | PZ-0112-P,1245,03/08/1999 |
| 641 | rat00100124 | 700067453 | ZS:221520.5 | RABTNOT01 | PZ-0001-P,1239,06/12/1997\|PZ-0001-US,1239,06/08/1998 |
| 642 | rat00000065 | 700478133 | ZS:221520.5 | RAFBNOT01 | PZ-0064-P,17,02/12/1998\|PZ-0064-US,17,02/10/1999 |
| 643 | rat00025121 | 700372405 | ZS:221520.5 | RARANOT01 | PZ-0037-US,1759,10/06/1998\|PZ-0037-P,570,10/09/1997 |
| 644 | rat00111342 | 700175226 | ZS:221520.5 | RASINOT01 | PZ-0005-P,118,07/09/1997\|PZ-0005-US,118,07/09/1998 |
| 645 | rat00012470 | 700060734 | ZS:221520.5 | RAKINOT02 | PZ-0002-US,1530,06/09/1998\|PZ-0002-P,788,06/13/1997 |
| 646 | rat00243312 | 700419934 | ZS:221520.5 | RAHANOT01 | PZ-0040-US,404,10/08/1998\|PZ-0041-P,404,10/09/1997 |
| 647 | rat00153024 | 701444306 | ZS:221520.5 | RALITXT41 | PZ-0123-P,1376,06/04/1999 |
| 648 | rat00093202 | 701720942 | ZS:221520.5 | RALIUNT15 | PZ-0129-P,71,06/18/1999 |
| 649 | rat00072036 | 700420845 | ZS:221520.5 | RAHANOT01 | PZ-0040-US,700,10/08/1998\|PZ-0041-P,700,10/09/1997 |
| 650 | rat00122347 | 700365506 | ZS:221520.5 | RAMGNOT01 | PZ-0046-P,269,11/04/1997\|PZ-0046-US,269,11/03/1998 |
| 651 | rat00128414 | 701474948 | ZS:221520.5 | RALITXT41 | PZ-0123-P,1005,06/04/1999 |
| 652 | rat00054660 | 700323207 | ZS:221520.5 | RAHINOT01 | PZ-0025-US,3133,09/09/1998\|PZ-0025-P,520,09/12/1997 |
| 653 | rat00160662 | 700067452 | ZS:221520.5 | RABTNOT01 | PZ-0001-P,1238,06/12/1997\|PZ-0001-US,1238,06/08/1998 |
| 654 | rat00243365 | 700376957 | ZS:221520.5 | RALANOT01 | PZ-0040-US,1820,10/08/1998\|PZ-0040-P,889,10/09/1997 |
| 655 | hu00195965 | 1307933 | IB:232035.4 | COLNFET02 | PD-0168-P,150,05/21/1996\|PD-0168-US,150,05/21/1997 |
| 656 | hu00492988 | 3974283 | IB:232035.4 | ADRETUT06 | PD-0333-US,3410,03/12/1998 |
| 657 | hu00410537 | 3422227 | IB:232035.4 | UCMCNOT04 | PD-0396-P,1291,07/03/1997\|PD-0396-US,1291,06/30/1998 |
| 658 | hu00133553 | 721822 | IB:232035.4 | SYNOOAT01 | PD-0099-US,2823,12/19/1996\|PD-0101-PD-0101-P,945,01/04/1996 |
| 659 | hu00341716 | 1839034 | IB:232035.4 | EOSITXT01 | PR-0011-P,736,08/29/1996\|PR-0011-US,736,08/29/1997 |
| 660 | hu00319153 | 3210659 | IB:232035.4 | BLADNOT08 | PD-0367-P,628,05/29/1997\|PD-0367-US,628,05/08/1998 |
| 661 | hu00890410 | 2226674 | IB:232035.4 | SEMVNOT01 | PD-0266-P,933,10/28/1996\|PD-0266-US,933,10/27/1997 |
| 662 | hu00761631 | 1432972 | IB:232035.4 | BEPINON01 | PD-0181-P,1302,05/30/1996\|PD-0181-US,1302,05/28/1997 |
| 663 | hu00424618 | 2243725 | IB:232035.4 | PANCTUT02 | PD-0295-P,3053,12/20/1996\|PD-0191-US,5211,06/19/1997 |
| 664 | hu00461663 | 2546759 | IB:232035.4 | UTRSNOT11 | PD-0316-P,3395,01/29/1997\|PD-0316-US,3395,01/26/1998 |
| 665 | hu00471664 | 259745 | IB:232035.4 | HNT2RAT01 | PD-0039-P,982,06/30/1995 |
| 666 | hu00175737 | 41970 | IB:232035.4 | TBLYNOT01 | PD-0007-US,1202,03/22/1994 |
| 667 | hu00080697 | 1592324 | IB:232035.4 | CARGNOT01 | PR-0010-P,570,06/27/1996\|PR-0010-US,570,06/26/1997 |
| 668 | hu00684950 | 3898065 | IB:232035.4 | TLYMNOT05 | PD-0364-US,3326,04/30/1998 |
| 669 | hu00910769 | 2735908 | IB:232035.4 | OVARNOT09 | PD-0311-P,478,01/27/1997\|PD-0311-US,478,01/23/1998 |
| 670 | hu00672773 | 2435175 | IB:232035.4 | BRAVUNT02 | PR-0012-P,1357,11/25/1996\|PR-0002-1-US,4391,11/19/1997 |
| 671 | hu00774949 | 2770076 | IB:232035.4 | COLANOT02 | PD-0321-P,923,02/12/1997\|PD-0321-P,923,02/12/1997\|PD-0321- |
| | | | | | US,923,02/10/1998\|PD-0321-US,923,02/10/1998 |
| 672 | hu00959976 | 1268496 | IB:232035.4 | BRAINOT09 | PD-0163-P,1025,05/21/1996\|PD-0163-US,1025,05/20/1997 |
| 673 | hu00124133 | 523940 | IB:232035.4 | MMLR2DT01 | PD-0044-P,2736,10/02/1995\|PD-0044-US,2736,10/02/1996 |
| 674 | hu00717302 | 2365873 | IB:232035.4 | ADRENOT07 | PD-0301-P,879,12/19/1996\|PD-0301-US,879,12/17/1997 |
| 675 | hu00552353 | 888182 | IB:232035.4 | PANCNOT05 | PD-0130-P,1053,03/26/1996 |
| 676 | hu00544172 | 2471553 | IB:232035.4 | THP1NOT03 | PD-0286-P,737,11/25/1996\|PD-0286-US,737,11/04/1997 |
| 677 | hu00943161 | 2360352 | IB:232035.4 | LUNGEET05 | PD-0281-P,843,12/04/1996 |
| 678 | hu00662183 | 2804594 | IB:232035.4 | BLADTUT08 | PD-0319-P,42,02/13/1997\|PD-0319-US,42,02/10/1998 |
| 679 | hu00590931 | 2684315 | IB:232035.4 | LUNGNOT23 | PD-0388-P,153,07/03/1997\|PD-0388-US,153,06/19/1998 |
| 680 | hu00235972 | 3181620 | IB:232035.4 | TLYJNOT01 | PD-0382-P,992,06/06/1997\|PD-0382-US,992,05/21/1998 |
| 681 | hu00867135 | 3566603 | IB:232035.4 | BRONNOT02 | PS-0003-P,941,06/06/1997\|PS-0003-US,941,06/06/1997 |
| 682 | hu00758833 | 3130394 | IB:232035.4 | LUNGTUT12 | PD-0358-P,1833,04/18/1997\|PD-0358-US,1833,04/17/1998 |
| 683 | hu00069795 | 735448 | IB:232035.4 | TONSNOT01 | PD-0100-P,557,01/05/1996\|PD-0100-US,557,01/03/1997 |
| 684 | hu00088668 | 2149570 | IB:232035.4 | BRAINOT09 | PD-0163-US,2428,05/20/1997\|PD-0275-P,394,12/06/1996 |
| 685 | hu00378757 | 1257772 | IB:232035.4 | MENITUT03 | PD-0147-P,1047,04/17/1996\|PD-0147-US,1047,04/17/1997 |
| 686 | hu00271021 | 2728309 | IB:232035.4 | OVARTUT05 | PD-0313-P,3373,01/22/1997\|PD-0313-US,3373,01/22/1998 |
| 687 | hu00238010 | 1468878 | IB:232035.4 | PANCTUT02 | PD-0191-P,882,06/21/1996\|PD-0191-US,882,06/19/1997 |
| 688 | hu00241262 | 4349373 | IB:232035.4 | TLYMTXT01 | PS-0008-P,1233,04/16/1998\|PS-0008-US,1233,04/16/1999 |
| 689 | hu00000477 | 2095174 | IB:232035.4 | BRAITUT02 | PD-0111-1-US,3450,12/05/1997\|PD-0275-P,3910,12/06/1996 |
| 690 | hu00432335 | 2588957 | IB:232035.4 | LUNGNOT22 | PD-0328-P,271,03/06/1997\|PD-0328-US,271,03/03/1998 |
| 691 | hu00015563 | 1991653 | IB:232035.4 | CORPNOT02 | PD-0256-P,1003,10/23/1996\|PD-0185-US,4823,06/26/1997 |
| 692 | hu00646150 | 2257385 | IB:232035.4 | OVARTUT01 | PD-0283-P,2827,11/21/1996\|PD-0112-1-US,4687,11/14/1997 |
| 693 | hu00062618 | 424822 | IB:232035.4 | BLADNOT01 | PD-0048-P,287,09/29/1995 |
| 694 | hu00091760 | 2764187 | IB:232035.4 | BRSTNOT12 | PD-0320-P,846,02/13/1997\|PD-0320-US,846,02/11/1998 |
| 695 | hu00466331 | 440571 | IB:232035.4 | THYRNOT01 | PD-0049-P,2748,10/02/1995\|PD-0049-US,2748,09/30/1996 |
| 696 | hu00624371 | 1451469 | IB:232035.4 | PENITUT01 | PD-0192-P,391,06/27/1996 |
| 697 | hu00613446 | 828745 | IB:232035.4 | PROSTUT04 | PD-0117-P,1654,02/26/1996\|PD-0117-US,1654,02/26/1997\|PD-0197- |
| | | | | | US,78,06/23/1997 |
| 698 | hu00107379 | 1982096 | IB:232035.4 | LUNGTUT03 | PD-0248-P,1129,09/27/1996\|PD-0189-US,2427,06/19/1997 |
| 699 | hu00736344 | 2986448 | IB:232035.4 | CARGDIT01 | PD-0349-P,647,03/31/1997\|PD-0349-US,647,03/31/1998 |
| 700 | hu00049527 | 3319382 | IB:232035.4 | PROSBPT03 | PD-0390-P,3211,06/30/1997\|PD-0390-US,3211,06/26/1998 |
| 701 | hu00123894 | 2681484 | IB:232035.4 | SINIUCT01 | PD-0330-P,862,02/12/1997\|PD-0330-US,862,02/10/1998 |
| 702 | hu00362585 | 2279834 | IB:232035.4 | PROSNON01 | PD-0198-US,4203,07/28/1997\|PD-0284-P,4203,12/05/1996 |
| 703 | hu00772940 | 3201139 | IB:232035.4 | PENCNOT02 | PD-0375-P,750,06/02/1997\|PD-0375-US,750,05/08/1998 |
| 704 | hu00567651 | 2696415 | IB:232035.4 | UTRSNOT12 | PD-0346-US,5220,03/27/1998\|PD-0346-P,938,03/27/1997 |
| 705 | hu00884551 | 79005 | IB:232035.4 | SYNORAB01 | PD-0012-US,1269,08/19/1994 |
| 706 | hu00217968 | 1307950 | IB:232035.4 | COLNFET02 | PD-0168-P,156,05/21/1996\|PD-0168-US,156,05/21/1997 |
| 707 | hu00653686 | 2322036 | IB:232035.4 | OVARNOT02 | PD-0293-P,1648,12/13/1996\|PD-0045-1-US,3475,11/10/1997 |
| 708 | hu00666277 | 2777457 | IB:232035.4 | OVARTUT03 | PD-0329-P,138,03/07/1997\|PD-0329-US,138,03/05/1998 |
| 709 | hu00682873 | 1801673 | IB:232035.4 | COLNNOT27 | PD-0277-US,1163,09/23/1997\|PD-0277-P,360,12/04/1996 |
| 710 | hu00901002 | 2232170 | IB:232035.4 | PROSNOT16 | PD-0290-P,1672,12/19/1996\|PD-0290-US,1672,07/30/1997 |
| 711 | hu00712113 | 1311164 | IB:232035.4 | COLNFET02 | PD-0168-P,1123,05/21/1996\|PD-0168-US,1123,05/21/1997 |
| 712 | hu00090046 | 4346781 | IB:232035.4 | TLYMTXT01 | PS-0008-P,445,04/16/1998\|PS-0008-US,445,04/16/1999 |
| 713 | hu00016543 | 1271781 | IB:232035.4 | TESTTUT02 | PD-0164-P,212,05/16/1996\|PD-0164-US,212,05/15/1997 |
| 714 | hu00102935 | 1451452 | IB:232035.4 | PENITUT01 | PD-0192-P,383,06/27/1996 |
| 715 | hu00170391 | 2503478 | IB:232035.4 | CONUTUT01 | PD-0300-P,563,12/20/1996\|PD-0300-US,563,12/18/1997 |
| 716 | hu00291433 | 2494513 | IB:232035.4 | ADRETUT05 | PD-0318-P,899,02/11/1997\|PD-0318-US,899,02/09/1998 |
| 717 | hu00433914 | 3325740 | IB:232035.4 | PTHYNOT03 | PD-0392-P,2338,07/01/1997\|PD-0392-US,2338,06/26/1998 |
| 718 | hu00379104 | 1505327 | IB:232035.4 | BRAITUT07 | PD-0183-P,1181,06/24/1996\|PD-0183-US,1181,06/18/1997 |
| 719 | hu00622960 | 1613557 | IB:232035.4 | COLNTUT06 | PD-0206-P,3884,08/01/1996\|PD-0206-US,3884,08/01/1997 |
| 720 | hu00316357 | 1254974 | IB:232035.4 | LUNGFET03 | PD-0146-P,4078,04/17/1996\|PD-0146-US,4078,04/16/1997 |
| 721 | hu00352638 | 2452005 | IB:232035.4 | ENDANOT01 | PD-0299-P,876,12/16/1996\|PD-0299-US,876,12/04/1997 |
| 722 | hu00542377 | 2612202 | IB:232035.4 | BRSTTUT13 | PD-0336-P,2,03/18/1997 |
| 723 | hu00484526 | 35154 | IB:232035.4 | HUVENOB01 | PD-0003-3-US,118,08/26/1994\|PD-0003-1-US,2702,02/17/1994\|PD- |
| | | | | | 0003-2-US,2839,04/28/1994 |
| 724 | hu00962138 | 3960285 | IB:232035.4 | HEARFET02 | PD-0353-US,3149,03/17/1998 |
| 725 | hu00961725 | 985780 | IB:232035.4 | LVENNOT03 | PD-0134-P,603,03/21/1996\|PD-0134-US,603,03/21/1997 |
| 726 | hu00545448 | 1839341 | IB:232035.4 | EOSITXT01 | PR-0011-P,839,08/29/1996\|PR-0011-US,839,08/29/1997 |
| 727 | hu00646249 | 1982581 | IB:232035.4 | LUNGTUT03 | PD-0248-P,1213,09/27/1996\|PD-0189-US,2511,06/19/1997 |
| 728 | hu00337964 | 1931150 | IB:232035.4 | COLNTUT03 | PD-0242-P,1308,09/25/1996\|PD-0174-US,2978,05/13/1997 |
| 729 | hu00854449 | 2452908 | IB:232035.4 | ENDANOT01 | PD-0299-P,1108,12/16/1996\|PD-0299-US,1108,12/04/1997 |
| 730 | hu01258892 | 6523007 | LG:197797.4 | CONFTDT02 | |
| 731 | hu01068980 | 6523607 | LG:197797.4 | CONFTDT02 | |
| 732 | rat00013687 | 700434903 | ZS:217422.1 | RAHLNOT02 | PZ-0047-P,64,11/06/1997\|PZ-0047-US,64,11/03/1998 |
| 733 | rat00197623 | 700934290 | ZS:217422.1 | RALINON03 | PZ-0075-P,1682,03/10/1998\|PZ-0075-US,2622,03/09/1999 |
| 734 | rat00079184 | 700418829 | ZS:217422.1 | RAHANOT01 | PZ-0040-US,62,10/08/1998\|PZ-0041-P,62,10/09/1997 |
| 735 | rat00097770 | 700249262 | ZS:217422.1 | RABHNOT02 | PZ-0021-US,3527,09/08/1998\|PZ-0021-P,951,09/09/1997 |
| 736 | rat00109965 | 700418821 | ZS:217422.1 | RAHANOT01 | PZ-0040-US,60,10/08/1998\|PZ-0041-P,60,10/09/1997 |
| 737 | rat00104653 | 700821102 | ZS:217422.1 | RAESNOT02 | PZ-0032-US,2024,10/07/1998\|PZ-0074-P,787,03/13/1998 |
| 738 | rat00142167 | 701580728 | ZS:217422.1 | RALIUNT03 | PZ-0121-P,406,05/03/1999 |
| 739 | rat00259341 | 700435587 | ZS:217422.1 | RAHLNOT02 | PZ-0047-P,303,11/06/1997\|PZ-0047-US,303,11/03/1998 |
| 740 | hu00601827 | 3328503 | IB:411605.4 | HEAONOT04 | PD-0387-P,3096,07/03/1997\|PD-0387-US,3096,06/30/1998 |
| 741 | hu00024989 | 40700 | IB:411605.6 | TBLYNOT01 | PD-0007-US,396,03/22/1994 |
| 742 | hu00999345 | 5678683 | LG:197797.8 | BRAENOT02 | |
| 743 | hu01011601 | 6432644 | LG:197797.8 | LUNGNON07 | |
| 744 | hu01143411 | 6010062 | LG:197797.8 | FIBRUNT02 | |
| 745 | hu01122354 | 6556049 | LG:197797.8 | BRAFNON02 | |
| 746 | hu01215505 | 6107822 | LG:197797.8 | MCLDTXT03 | |
| 747 | hu01004563 | 6078778 | LG:197797.8 | BMARUNT02 | |
| 748 | hu01075341 | 6381042 | LG:197797.8 | FIBRUNT02 | |
| 749 | hu01271901 | 6612661 | LG:197797.8 | ARTANOT07 | |
| 750 | hu01189121 | 6544335 | LG:197797.8 | LNODNON02 | |
| 751 | hu01263266 | 6429658 | LG:197797.8 | LUNGNON07 | |
| 752 | hu01258550 | 5877503 | LG:197797.8 | BRAUNOT01 | |
| 753 | hu01091807 | 6603752 | LG:197797.8 | UTREDIT07 | |
| 754 | hu01325277 | 6564202 | LG:197797.8 | MCLDTXT04 | |
| 755 | hu01072756 | 6514218 | LG:197797.8 | THYMDIT01 | |
| 756 | hu01305668 | 4519243 | LG:197797.8 | SINJNOT03 | |
| 757 | hu01034966 | 4519149 | LG:197797.8 | SINJNOT03 | |
| 758 | hu01268635 | 6402167 | LG:197797.8 | UTRENOT10 | |
| 759 | hu00967441 | 3162188 | LG:197797.8 | TLYMTXT03 | |
| 760 | hu01185674 | 6544193 | LG:197797.8 | LNODNON02 | |
| 761 | hu01089257 | 5564022 | LG:197797.8 | BRSTDIT01 | |
| 762 | hu01070889 | 3762964 | LG:197797.8 | BRAHDIT03 | |
| 763 | hu00396958 | 85665 | IB:411605.7 | LIVRNOT01 | PD-0014-US,461,09/07/1994 |
| 764 | hu00230202 | 78988 | IB:411605.7 | SYNORAB01 | PD-0012-US,1255,08/19/1994 |
| 765 | hu00300233 | 72104 | IB:411605.7 | | PD-0001-4-US,59,07/28/1994 |
| 766 | hu00433221 | 69718 | IB:411605.7 | HUVESTB01 | PD-0003-2-US,7143,04/28/1994\|PD-0003-3-US,8259,08/26/1994 |
| 767 | hu00150706 | 649490 | IB:411605.7 | CARCTXT02 | PR-0001-P,1115,11/17/1995\|PR-0001-US,1115,11/15/1996 |
| 768 | hu00270623 | 26609 | IB:411605.7 | SPLNFET01 | PD-0004-US,1002,02/02/1994\|PD-0004-US,1002,02/02/1994 |
| 769 | hu00119128 | 41719 | IB:411605.7 | TBLYNOT01 | PD-0007-US,1016,03/22/1994 |
| 770 | hu00608710 | 77013 | IB:411605.7 | SYNORAB01 | PD-0012-US,10,08/19/1994 |
| 771 | hu00804308 | 1862465 | IB:411605.7 | PROSNOT19 | |
| 772 | hu00284711 | 3179407 | IB:411605.7 | TLYJNOT01 | PD-0382-P,388,06/06/1997\|PD-0382-US,388,05/21/1998 |
| 773 | hu00490282 | 68007 | IB:411605.7 | HUVESTB01 | PD-0003-2-US,6562,04/28/1994\|PD-0003-3-US,7678,08/26/1994 |
| 774 | hu00139714 | 3079952 | IB:411605.7 | BRAIUNT01 | PD-0348-P,612,03/31/1997\|PD-0348-US,612,03/30/1998 |
| 775 | hu00231892 | 892 | IB:411605.7 | U937NOT01 | PD-0001-3-US,723,02/14/1994\|PD-0001-US,723,07/17/1992\|PD- |
| | | | | | 0001-1-PCT,814,07/16/1993\|PD-0001-1-US,814,11/19/1992 |
| 776 | hu00357416 | 581245 | IB:411605.7 | BRAVTXT05 | PR-0003-US,2393,11/13/1996\|PR-0004-P,2393,12/15/1995 |
| 777 | hu00515780 | 1416 | IB:411605.7 | U937NOT01 | PD-0001-1-PCT,1297,07/16/1993\|PD-0001-1- |
| | | | | | US,1297,11/19/1992\|PD-0001-3-US,1297,02/14/1994 |
| 778 | hu00620289 | 536352 | IB:411605.7 | ADRENOT03 | PD-0144-US,1490,04/10/1997\|PD-0055-P,255,10/24/1995\|PD-0055- |
| | | | | | US,255,10/16/1996 |
| 779 | hu00634536 | 4213912 | IB:411605.7 | BRONDIT01 | PS-0006-P,1967,12/23/1997\|PS-0004-US,2916,12/13/1996 |
| 780 | hu00295381 | 52704 | IB:411605.7 | | PD-0008-US,224,06/27/1994 |
| 781 | hu00689090 | 2914553 | IB:411605.7 | THYMFET03 | PD-0345-P,232,03/21/1997\|PD-0345-US,232,03/18/1998 |
| 782 | hu00624894 | 578971 | IB:411605.7 | BRAVTXT05 | PR-0003-US,1734,11/13/1996\|PR-0004-P,1734,12/15/1995 |
| 783 | rat00180808 | 700768273 | ZS:220379.1 | RAHYNOT02 | PZ-0078-P,1373,04/06/1998\|PZ-0033-US,3037,10/05/1998 |
| 784 | rat00029077 | 701242681 | ZS:220379.1 | RALINOH02 | PZ-0091-P,980,10/27/1998 |
| 785 | rat00238831 | 701246365 | ZS:220379.1 | RALINOH02 | PZ-0091-P,2355,10/27/1998 |
| 786 | rat00157148 | 701188745 | ZS:220379.1 | RACONON02 | PZ-0085-P,1468,06/11/1998 |
| 787 | rat00228376 | 700496220 | ZS:220379.1 | RAHINOT01 | PZ-0025-US,342,09/09/1998\|PZ-0044-P,342,11/04/1997 |
| 788 | rat00112998 | 700370466 | ZS:220379.1 | RAHYNOT01 | PZ-0033-P,1505,10/08/1997\|PZ-0033-US,1505,10/05/1998 |
| 789 | rat00067297 | 700315154 | ZS:220379.1 | RAESNOT01 | PZ-0032-P,722,10/08/1997\|PZ-0032-US,722,10/07/1998 |
| 790 | rat00099177 | 700497170 | ZS:220379.1 | RAHINOT01 | PZ-0025-US,829,09/09/1998\|PZ-0044-P,829,11/04/1997 |
| 791 | rat00188749 | 700770692 | ZS:220379.1 | RABONOT01 | PZ-0076-US,2604,04/06/1999\|PZ-0077-P,300,04/07/1998 |
| 792 | rat00149897 | 700290724 | ZS:220379.1 | RAEPNOT01 | PZ-0015-P,706,08/05/1997\|PZ-0015-US,706,08/04/1998 |
| 793 | rat00102720 | 700367624 | ZS:220379.1 | RAHYNOT01 | PZ-0033-P,126,10/08/1997\|PZ-0033-US,126,10/05/1998 |
| 794 | rat00119114 | 700066526 | ZS:220379.1 | RABTNOT01 | PZ-0001-P,909,06/12/1997\|PZ-0001-US,909,06/08/1998 |
| 795 | rat00214272 | 700061414 | ZS:220379.1 | RAKINOT02 | PZ-0002-P,1061,06/13/1997\|PZ-0002-US,1803,06/09/1998 |
| 796 | rat00166231 | 701482761 | ZS:220379.1 | RALITXT42 | PZ-0109-P,1018,02/11/1999 |
| 797 | rat00071074 | 700541247 | ZS:220379.1 | RACONOT01 | PZ-0043-P,1407,11/04/1997\|PZ-0014-US,2845,08/05/1998 |
| 798 | rat00240806 | 701193468 | ZS:220379.1 | RALINOT02 | PZ-0026-US,1543,09/04/1998\|PZ-0082-P,213,05/01/1998 |
| 799 | rat00017707 | 700606709 | ZS:220379.1 | RALINOH01 | PZ-0053-P,110,12/12/1997\|PZ-0053-US,110,12/11/1998 |
| 800 | rat00103279 | 700420587 | ZS:220379.1 | RAHANOT01 | PZ-0040-US,620,10/08/1998\|PZ-0041-P,620,10/09/1997 |
| 801 | rat00179213 | 700593713 | ZS:220379.1 | RATRNOT04 | PZ-0062-P,1327,01/16/1998 |
| 802 | rat00155382 | 700928945 | ZS:220379.1 | RACONON04 | PZ-0085-P,3819,06/11/1998 |
| 803 | rat00241944 | 701189016 | ZS:220379.1 | RACONON02 | PZ-0085-P,1629,06/11/1998 |
| 804 | hu00788751 | 1986692 | IB:902506.4 | LUNGAST01 | PD-0120-US,4303,02/28/1997\|PD-0247-P,957,09/27/1996 |
| 805 | hu00263898 | 2270579 | IB:902506.4 | PROSNON01 | PD-0198-US,118,07/28/1997\|PD-0284-P,118,12/05/1996 |
| 806 | hu00679088 | 3274021 | IB:902506.4 | PROSBPT06 | PD-0376-P,639,06/02/1997\|PD-0376-US,639,05/15/1998 |
| 807 | hu00411401 | 1347425 | IB:902506.4 | PROSNOT11 | PD-0175-P,1177,05/24/1996\|PD-0175-US,1177,05/23/1997 |
| 808 | hu00849869 | 864155 | IB:902506.4 | BRAITUT03 | PD-0126-1-US,2148,11/25/1997\|PD-0126-P,2148,03/08/1996\|PD- |
| | | | | | 0126-US,2148,03/07/1997 |
| 809 | hu00733110 | 1423768 | IB:902506.4 | CARGDIT02 | PR-0008-P,1063,06/04/1996\|PR-0008-US,1063,06/03/1997 |
| 810 | hu00500094 | 2355551 | IB:902506.4 | LUNGNOT20 | PD-0282-P,630,12/04/1997\|PD-0282-US,630,11/14/1997 |
| 811 | hu00630613 | 3325114 | IB:902506.4 | PTHYNOT03 | PD-0392-P,2054,07/01/1997\|PD-0392-US,2054,06/26/1998 |
| 812 | hu00483211 | 1296794 | IB:902506.4 | PGANNOT03 | PD-0165-P,1588,05/28/1996\|PD-0165-US,1588,05/23/1997 |
| 813 | hu00726024 | 1340754 | IB:902506.4 | COLNTUT03 | PD-0174-US,1448,05/13/1997\|PD-0174-P,737,05/13/1996 |
| 814 | hu00804640 | 2695839 | IB:902506.4 | UTRSNOT12 | PD-0346-US,5009,03/27/1998\|PD-0346-P,731,03/27/1997 |
| 815 | hu00434840 | 1746423 | IB:902506.4 | STOMTUT02 | PD-0235-P,229,08/30/1996 |
| 816 | hu00307931 | 1502674 | IB:902506.4 | BRAITUT07 | PD-0183-P,112,06/24/1996\|PD-0183-US,112,06/18/1997 |
| 817 | hu00118249 | 2899137 | IB:902506.4 | DRGCNOT01 | PD-0337-US,4433,03/05/1998 |
| 818 | hu00359170 | 1294153 | IB:902506.4 | PGANNOT03 | PD-0165-P,756,05/28/1996\|PD-0165-US,756,05/23/1997 |
| 819 | hu00834424 | 3256826 | IB:902506.4 | OVARTUN01 | PD-0374-P,2112,06/04/1997\|PD-0374-US,2112,05/18/1998 |
| 820 | hu00491309 | 3244929 | IB:902506.4 | BRAINOT19 | PD-0370-P,1299,05/29/1997\|PD-0370-US,1299,05/12/1998 |
| 821 | hu00641394 | 1739432 | IB:902506.4 | HIPONON01 | PD-0224-P,69,08/28/1996\|PD-0224-US,69,08/26/1997 |
| 822 | hu00529683 | 3116132 | IB:902506.4 | LUNGTUT13 | PD-0358-P,2112,04/18/1997\|PD-0358-US,2112,04/17/1998 |
| 823 | hu00237499 | 1386061 | IB:902506.4 | CARGDIT02 | PR-0008-P,131,06/04/1996\|PR-0008-US,131,06/03/1997 |
| 824 | hu00697147 | 3337508 | IB:902506.4 | SPLNNOT10 | PD-0378-P,2315,06/05/1997\|PD-0378-US,2315,05/19/1998 |
| 825 | hu00197335 | 1905370 | IB:334807.1 | OVARNOT07 | PD-0249-P,2976,10/01/1996 |
| 826 | hu00731987 | 2050014 | IB:411449.5 | LIVRFET02 | PD-0262-P,514,10/31/1996 |
| 827 | hu00795629 | 1972386 | IB:411449.5 | UCMCL5T01 | PD-0194-US,3338,06/27/1997\|PD-0272-P,737,10/23/1996 |
| 828 | hu00885639 | 463172 | IB:411449.5 | LATRNOT01 | PD-0053-P,2675,10/10/1995\|PD-0053-US,2675,10/09/1996 |
| 829 | hu00728978 | 1490003 | IB:411449.5 | UCMCL5T01 | PD-0194-P,2147,06/28/1996\|PD-0194-US,2147,06/27/1997\|PD-0184- |
| | | | | | P,823,06/24/1996 |
| 830 | hu00531230 | 239023 | IB:411449.5 | HIPONOT01 | PD-0026-US,6,02/23/1995 |
| 831 | hu00502701 | 2096680 | IB:411449.5 | BRAITUT02 | PD-0111-1-US,3959,12/05/1997\|PD-0275-P,4419,12/06/1996 |
| 832 | hu00125531 | 2910217 | IB:411449.5 | KIDNTUT15 | |
| 833 | hu00161830 | 1698871 | IB:411449.5 | BLADTUT05 | PD-0221-US,466,07/31/1997\|PD-0202-P,873,07/31/1996 |
| 834 | hu00113816 | 2472307 | IB:411449.5 | THP1NOT03 | PD-0286-P,971,11/25/1996\|PD-0286-US,971,11/04/1997 |
| 835 | hu00763211 | 1689376 | IB:411449.5 | PROSTUT10 | PD-0200-P,2956,07/24/1996\|PD-0200-US,2956,07/24/1997 |
| 836 | hu00623901 | 995171 | IB:411449.5 | KIDNTUT01 | PD-0137-P,37,03/25/1996\|PD-0137-US,37,03/25/1997 |
| 837 | hu00447421 | 3323088 | IB:411449.5 | PTHYNOT03 | PD-0392-P,1105,07/01/1997\|PD-0392-US,1105,06/26/1998 |
| 838 | hu00550627 | 2758260 | IB:411449.5 | THP1AZS08 | PD-0362-P,4482,05/01/1997\|PD-0362-US,4482,04/29/1998 |
| 839 | hu00740186 | 3448616 | IB:411449.5 | UTRSNON03 | PD-0397-P,510,07/02/1997\|PD-0397-US,510,06/30/1998 |
| 840 | hu00440838 | 3292975 | IB:411449.5 | BONRFET01 | PD-0368-P,1483,05/28/1997\|PD-0368-US,1483,05/05/1998 |
| 841 | hu00223687 | 763788 | IB:411449.5 | LUNGNOT04 | PD-0121-US,1454,02/28/1997\|PD-0140-P,7,04/10/1996 |
| 842 | hu00872394 | 2476089 | IB:411449.5 | SMCANOT01 | PD-0298-P,452,01/30/1998\|PD-0298-US,452,01/30/1998 |
| 843 | hu00834378 | 3451002 | IB:411449.5 | UTRSNON03 | PD-0397-P,1853,07/02/1997\|PD-0397-US,1853,06/30/1998 |
| 844 | hu00554569 | 1625423 | IB:411449.5 | COLNPOT01 | PD-0206-P,2062,08/01/1996\|PD-0206-US,2062,08/01/1997 |
| 845 | hu00743996 | 2682429 | IB:411449.5 | SINIUCT01 | PD-0330-P,1155,02/12/1997\|PD-0330-US,1155,02/10/1998 |
| 846 | hu00919779 | 491356 | IB:411449.5 | HNT2AGT01 | |
| 847 | hu00107621 | 1811236 | IB:411449.5 | PROSTUT12 | PD-0233-P,4420,09/05/1996\|PD-0314-US,982,09/04/1997 |
| 848 | hu00321704 | 2598057 | IB:411449.5 | OVARTUT02 | PD-0312-P,1654,01/21/1997\|PD-0312-US,1654,01/21/1998 |
| 849 | hu00104670 | 1984010 | IB:411449.5 | LUNGAST01 | PD-0120-US,3384,02/28/1997\|PD-0247-P,38,09/27/1996 |
| 850 | hu00102690 | 2830524 | IB:411449.5 | TLYMNOT03 | PD-0363-P,1463,05/02/1997\|PD-0363-US,1463,04/30/1998 |
| 851 | hu00527304 | 8248 | IB:411449.5 | | |
| 852 | hu00017059 | 321613 | IB:411449.5 | EOSIHET02 | PD-0067-P,3891,09/29/1995 |
| 853 | hu00850406 | 677686 | IB:411449.5 | CRBLNOT01 | PD-0093-P,1614,12/18/1995\|PD-0089-US,3310,11/27/1996 |
| 854 | hu00793203 | 2584176 | IB:411449.5 | BRAITUT22 | PD-0304-P,3154,01/28/1997\|PD-0304-US,3154,01/28/1998 |
| 855 | hu00732715 | 638241 | IB:411449.5 | BRSTNOT03 | PD-0061-US,286,11/15/1996\|PD-0065-P,286,11/28/1995 |
| 856 | hu00242629 | 257820 | IB:411449.5 | HNT2RAT01 | PD-0039-P,415,06/30/1995 |
| 857 | hu00591902 | 506236 | IB:411449.5 | TMLR3DT02 | PD-0081-P,1131,10/25/1995\|PD-0081-US,1131,10/24/1996 |
| 858 | hu00611478 | 2645110 | IB:411449.5 | OVARTUT03 | PD-0329-P,2367,03/07/1997\|PD-0329-US,2367,03/05/1998 |
| 859 | hu00514953 | 1957671 | IB:411449.5 | CONNNOT01 | PD-0244-P,1161,10/04/1996\|PD-0244-US,1161,10/03/1997 |
| 860 | hu00847134 | 2682468 | IB:411449.5 | SINIUCT01 | PD-0330-P,1176,02/12/1997\|PD-0330-US,1176,02/10/1998 |
| 861 | hu00527429 | 1302 | IB:411449.5 | U937NOT01 | PD-0001-1-PCT,1194,07/16/1993\|PD-0001-1- |
| | | | | | US,1194,11/19/1992\|PD-0001-3-US,1194,02/14/1994 |
| 862 | hu00778152 | 3361311 | IB:411449.5 | PROSBPT02 | PD-0390-P,227,06/30/1997\|PD-0390-US,227,06/26/1998 |
| 863 | hu00483538 | 1355750 | IB:411449.5 | LUNGNOT09 | PD-0177-P,400,05/15/1996\|PD-0177-US,400,05/14/1997 |
| 864 | hu00169762 | 2173973 | IB:411449.5 | ENDCNOT03 | PD-0279-P,2047,11/22/1996\|PD-0279-US,2047,11/07/1997 |
| 865 | hu00627494 | 57539 | IB:411449.5 | | PD-0009-US,174,07/08/1994 |
| 866 | hu00683672 | 2613128 | IB:411449.5 | BRSTTUT14 | PD-0320-P,4282,02/13/1997\|PD-0320-US,4282,02/11/1998 |
| 867 | hu00619358 | 2782774 | IB:411449.5 | BRSTNOT13 | PD-0335-P,216,03/21/1997\|PD-0335-US,216,03/20/1998 |
| 868 | hu00334846 | 1703167 | IB:334807.6 | DUODNOT02 | PD-0207-P,1739,07/29/1996\|PD-0207-US,1739,07/29/1997 |
| 869 | hu00538435 | 806627 | IB:334807.6 | BSTMNOT01 | PD-0115-P,548,02/27/1996\|PD-0115-US,548,02/27/1997 |
| 870 | hu00045975 | 1831627 | IB:334807.6 | THP1AZT01 | PD-0236-P,683,08/30/1996\|PD-0236-US,683,08/29/1997 |
| 871 | hu00255713 | 381954 | IB:334807.6 | HYPONOB01 | PD-0071-P,446,10/03/1995 |
| 872 | hu00309830 | 3245690 | IB:334807.6 | BRAINOT19 | PD-0370-P,1641,05/29/1997\|PD-0370-US,1641,05/12/1998 |
| 873 | hu00838320 | 1593378 | IB:334807.6 | BRAINOT14 | PD-0204-P,166,07/26/1996 |
| 874 | hu00600416 | 928044 | IB:334807.6 | BRAINOT04 | PD-0132-P,1181,03/18/1996\|PD-0132-US,1181,03/17/1997 |
| 875 | hu00678814 | 1627474 | IB:334807.6 | COLNPOT01 | PD-0206-P,2480,08/01/1996\|PD-0206-US,2480,08/01/1997 |
| 876 | hu00220365 | 2750679 | IB:334807.6 | THP1AZS08 | PD-0362-P,562,05/01/1997\|PD-0362-US,562,04/29/1998 |
| 877 | hu00715266 | 691867 | IB:902506.9 | LUNGTUT02 | PD-0097-P,1119,12/20/1995\|PD-0097-US,1119,12/20/1996 |
| 878 | hu00061515 | 688682 | IB:902506.9 | LUNGTUT02 | PD-0097-P,92,12/20/1995\|PD-0097-US,92,12/20/1996 |
| 879 | hu01256988 | 5865786 | LG:034727.1 | COLTDIT04 | |
| 880 | hu01320806 | 6179644 | LG:244682.1 | BMARUNT02 | |
| 881 | hu00225272 | 1903494 | IB:815587.1 | OVARNOT07 | PD-0249-P,2256,10/01/1996 |
| 882 | hu00492301 | 434242 | IB:399851.1 | THYRNOT01 | PD-0049-P,401,10/02/1995\|PD-0049-US,401,09/30/1996 |
| 883 | hu00147708 | 3139053 | IB:399851.1 | SMCCNOT02 | PD-0377-P,1740,06/06/1997\|PD-0377-US,1740,05/20/1998 |
| 884 | hu00720528 | 864652 | IB:399851.1 | BRAITUT03 | PD-0126-1-US,2336,11/25/1997\|PD-0126-P,2336,03/08/1996\|PD- |
| | | | | | 0126-US,2336,03/07/1997 |
| 885 | hu00662648 | 631104 | IB:399851.1 | KIDNNOT05 | PD-0063-P,1223,11/22/1995\|PD-0261-1-US,1223,10/29/1997 |
| 886 | hu00425257 | 1729479 | IB:399851.1 | BRSTTUT08 | PD-0225-P,217,08/28/1996 |
| 887 | hu01145798 | 6423435 | LG:459192.4 | BRSTUNT01 | |
| 888 | hu00990657 | 5712404 | LG:459192.4 | MASTTXT01 | |
| 889 | hu01033267 | 6100191 | LG:459192.4 | UTRENOT09 | |
| 890 | hu01064572 | 6096811 | LG:459192.4 | THP1TXT03 | |
| 891 | hu01014659 | 6411280 | LG:459192.4 | UTREDIT10 | |
| 892 | hu01343943 | 6514626 | LG:459192.4 | THYMDIT01 | |
| 893 | hu01172385 | 3787301 | LG:459192.4 | BRAHNOT05 | |
| 894 | hu01274383 | 6565167 | LG:459192.4 | MCLDTXT04 | |
| 895 | hu01126099 | 4667847 | LG:459192.4 | SINTNOT24 | |
| 896 | hu01096453 | 6411253 | LG:459192.4 | UTREDIT10 | |
| 897 | hu01280694 | 5842026 | LG:459192.4 | BRAENOT04 | |
| 898 | hu01303215 | 4518594 | LG:459192.4 | SINJNOT03 | |
| 899 | hu01082041 | 6541505 | LG:459192.4 | LNODNON02 | |
| 900 | hu00153508 | 2597277 | IB:197797.5 | OVARTUT02 | PD-0312-P,1355,01/21/1997\|PD-0312-US,1355,01/21/1998 |
| 901 | hu00928636 | 923474 | IB:197797.5 | RATRNOT02 | PD-0135-P,1053,03/20/1996\|PD-0135-US,1053,03/19/1997 |
| 902 | hu00498907 | 1475844 | IB:197797.6 | LUNGTUT03 | PD-0189-US,1163,06/19/1997\|PD-0189-P,1183,06/20/1996 |
| 903 | hu01128530 | 5618571 | LG:449795.1 | THYMNOR02 | |
| 904 | hu01111791 | 6246764 | LG:449795.1 | TESTNOT17 | |
| 905 | hu01274800 | 5700115 | LG:449795.1 | DRGCNOT01 | |
| 906 | hu01075270 | 5951238 | LG:449795.1 | LIVRTUN04 | |
| 907 | hu01040299 | 6390391 | LG:449795.1 | BONRNOT01 | |
| 908 | hu01234846 | 6390312 | LG:449795.1 | BONRNOT01 | |
| 909 | hu01296534 | 3567447 | LG:449795.1 | BRONNOT02 | |
| 910 | hu01176871 | 6247056 | LG:449795.1 | TESTNOT17 | |
| 911 | hu00970376 | 5615260 | LG:449795.1 | THYMNOR02 | |
| 912 | hu00188328 | 1786291 | IB:197797.8 | BRAINOT10 | PD-0239-P,777,09/27/1996\|PD-0239-US,777,09/24/1997 |
| 913 | hu00512707 | 2779480 | IB:197797.8 | OVARTUT03 | PD-0329-P,1086,03/07/1997\|PD-0329-US,1086,03/05/1998 |
| 914 | hu00458446 | 1315024 | IB:197797.8 | BLADTUT02 | PD-0171-P,620,05/23/1996\|PD-0171-US,620,05/22/1997 |
| 915 | hu00310776 | 1786745 | IB:197797.8 | BRAINOT10 | PD-0239-P,956,09/27/1996\|PD-0239-US,956,09/24/1997 |
| 916 | hu00878881 | 1258759 | IB:197797.8 | MENITUT03 | PD-0147-P,1482,04/17/1996\|PD-0147-US,1482,04/17/1997 |
| 917 | hu00695058 | 1547727 | IB:197797.8 | PROSTUT04 | PD-0197-P,1890,06/21/1996\|PD-0197-US,3425,06/23/1997 |
| 918 | hu00809565 | 2100659 | IB:197797.8 | BRAITUT02 | PD-0111-1-US,5409,12/05/1997\|PD-0275-P,5869,12/06/1996 |
| 919 | hu00422460 | 2839374 | IB:197797.8 | DRGLNOT01 | PD-0337-P,853,03/18/1997\|PD-0337-US,853,03/05/1998 |
| 920 | hu00236474 | 1634458 | IB:197797.8 | COLNNOT19 | PD-0206-P,698,08/01/1996\|PD-0206-US,698,08/01/1997 |
| 921 | hu00504324 | 2702257 | IB:197797.8 | OVARTUT10 | PD-0341-P,3530,03/20/1997\|PD-0341-US,3530,03/19/1998 |
| 922 | hu00665846 | 2356088 | IB:230901.1 | LUNGNOT20 | PD-0282-P,787,12/04/1997\|PD-0282-US,787,11/14/1997 |
| 923 | hu00369297 | 978769 | IB:230901.1 | BRSTNOT02 | PD-0157-P,1805,04/26/1996\|PD-0157-US,1805,04/25/1997 |
| 924 | hu00729723 | 2654856 | IB:230901.1 | THYMNOT04 | PD-0315-P,4208,01/30/1997\|PD-0315-US,4208,01/30/1998 |
| 925 | hu00228581 | 3181917 | IB:230901.1 | TLYJNOT01 | PD-0382-P,1089,06/06/1997\|PD-0382-US,1089,05/21/1998 |
| 926 | hu00747970 | 2016903 | IB:230901.1 | THP1NOT01 | PD-0273-US,1443,10/28/1997\|PD-0273-P,147,10/31/1996 |
| 927 | rat00217853 | 701230984 | ZS:212821.1 | RASJNON03 | PZ-0098-P,950,11/12/1998 |
| 928 | rat00105569 | 700627723 | ZS:212821.1 | RAFBNOT01 | PZ-0064-P,1020,02/12/1998\|PZ-0064-US,1020,02/10/1999 |
| 929 | rat00066626 | 700911955 | ZS:212821.1 | RAXSNOT02 | PZ-0100-P,2668,11/12/1998 |
| 930 | rat00209690 | 701280056 | ZS:212821.1 | RABXNOT02 | PZ-0087-P,313,08/11/1998 |
| 931 | rat00193805 | 700769886 | ZS:212821.1 | RAHYNOT02 | PZ-0078-P,2042,04/06/1998\|PZ-0033-US,3706,10/05/1998 |
| 932 | rat00080783 | 700647424 | ZS:212821.1 | RAPINOT02 | PZ-0065-P,40,02/11/1998\|PZ-0065-US,40,02/10/1999 |
| 933 | rat00177525 | 701396532 | ZS:212821.1 | RALITXT14 | PZ-0094-P,2968,09/11/1998 |
| 934 | rat00068166 | 700490877 | ZS:212821.1 | RABCNOT01 | PZ-0012-US,2025,08/04/1998\|PZ-0042-P,224,11/05/1997 |
| 935 | rat00129844 | 700374582 | ZS:212821.1 | RALANOT01 | PZ-0040-US,1135,10/08/1998\|PZ-0040-P,204,10/09/1997 |
| 936 | rat00159187 | 700373236 | ZS:212821.1 | RARANOT01 | PZ-0037-US,2040,10/06/1998\|PZ-0037-P,851,10/09/1997 |
| 937 | rat00126977 | 700546735 | ZS:212821.1 | RASPNOT01 | PZ-0055-P,1423,12/12/1997\|PZ-0003-US,3602,05/21/1998 |
| 938 | rat00085597 | 701194114 | ZS:212821.1 | RALINOT02 | PZ-0026-US,1697,09/04/1998\|PZ-0082-P,367,05/01/1998 |
| 939 | rat00002212 | 701644894 | ZS:212821.1 | RALITXT40 | PZ-0120-P,434,05/06/1999 |
| 940 | rat00240035 | 700126003 | ZS:212821.1 | RABHNOT01 | PZ-0004-P,1788,07/09/1997\|PZ-0004-US,1788,07/09/1998 |
| 941 | rat00004910 | 700642857 | ZS:212821.1 | RABUNOT01 | PZ-0058-P,1247,01/16/1998\|PZ-0057-US,3605,01/11/1999 |
| 942 | rat00150261 | 701512158 | ZS:212821.1 | RALITXT02 | PZ-0112-P,1886,03/08/1999 |
| 943 | rat00106163 | 700479909 | ZS:212821.1 | RALINON03 | PZ-0059-P,89,01/16/1998\|PZ-0059-US,89,01/12/1999 |
| 944 | rat00035083 | 700059536 | ZS:212821.1 | RAKINOT02 | PZ-0002-US,1020,06/09/1998\|PZ-0002-P,278,06/13/1997 |
| 945 | rat00203069 | 700629237 | ZS:212821.1 | RAFBNOT01 | PZ-0064-P,1498,02/12/1998\|PZ-0064-US,1498,02/10/1999 |
| 946 | rat00028253 | 701304081 | ZS:212821.1 | RASGNON05 | PZ-0105-P,652,12/10/1998 |
| 947 | rat00078430 | 701651203 | ZS:212821.1 | RALITXT10 | PZ-0117-P,566,05/07/1999 |
| 948 | rat00056155 | 700505437 | ZS:212821.1 | RALINON07 | PZ-0060-P,2054,01/16/1998\|PZ-0059-US,4252,01/12/1999 |
| 949 | rat00104549 | 701484590 | ZS:212821.1 | RALITXT50 | PZ-0109-P,1369,02/11/1999 |
| 950 | rat00108680 | 701026436 | ZS:212821.1 | RAFANOT02 | PZ-0101-P,966,12/10/1998 |
| 951 | rat00127454 | 701467315 | ZS:212821.1 | RALITXT32 | PZ-0108-P,693,02/11/1999 |
| 952 | rat00081843 | 701484801 | ZS:212821.1 | RALITXT50 | PZ-0109-P,1412,02/11/1999 |
| 953 | rat00137590 | 700911350 | ZS:212821.1 | RAXSNOT02 | PZ-0100-P,2438,11/12/1998 |
| 954 | rat00190565 | 700461547 | ZS:212821.1 | RATHNOC02 | PZ-0049-P,92,11/07/1997\|PZ-0049-US,92,11/09/1998 |
| 955 | rat00013868 | 700493031 | ZS:212821.1 | RABCNOT01 | PZ-0042-P,1273,11/05/1997\|PZ-0012-US,3074,08/04/1998 |
| 956 | rat00162269 | 701490669 | ZS:212821.1 | RALITXT54 | PZ-0135-P,520,08/05/1999 |
| 957 | rat00088471 | 700514441 | ZS:212821.1 | RASNNOT01 | PZ-0054-P,1590,12/11/1997\|PZ-0054-US,1590,12/10/1998 |
| 958 | rat00093566 | 701025551 | ZS:212821.1 | RAFANOT02 | PZ-0101-P,892,12/10/1998 |
| 959 | rat00042779 | 700396312 | ZS:212821.1 | RAHTNOT01 | PZ-0037-US,50,10/06/1998\|PZ-0048-P,50,11/05/1997 |
| 960 | rat00017885 | 701485302 | ZS:212821.1 | RALITXT50 | P7-0109-P,1514,02/11/1999 |
| 961 | rat00171598 | 701913515 | ZS:212821.1 | RABYUNS03 | PZ-0125-P,853,06/29/1999 |
| 962 | rat00189715 | 701021709 | ZS:212821.1 | RAFANOT02 | PZ-0101-P,87,12/10/1998 |
| 963 | rat00053789 | 701186432 | ZS:212821.1 | RACONON02 | PZ-0085-P,67,06/11/1998 |
| 964 | rat00175856 | 700310390 | ZS:212821.1 | RAUCNOT01 | PZ-0029-P,230,09/11/1997\|PZ-0029-US,230,09/04/1998 |
| 965 | rat00164967 | 700591163 | ZS:212821.1 | RATRNOT04 | PZ-0062-P,559,01/16/1998 |
| 966 | rat00078693 | 701290475 | ZS:212821.1 | RABXNOT04 | PZ-0090-P,317,08/12/1998 |
| 967 | rat00066877 | 700639812 | ZS:212821.1 | RATONOT01 | PZ-0036-US,1299,10/09/1998 |
| 968 | rat00096796 | 700052543 | ZS:212821.1 | RABTNOT01 | PZ-0001-P,8,06/12/1997\|PZ-0001-US,8,06/08/1998 |
| 969 | rat00161402 | 701022008 | ZS:212821.1 | RAFANOT02 | PZ-0101-P,181,12/10/1998 |
| 970 | rat00161602 | 701468265 | ZS:212821.1 | RALITXT32 | PZ-0108-P,887,02/11/1999 |
| 971 | rat00010529 | 700769478 | ZS:212821.1 | RAHYNOT02 | PZ-0078-P,1872,04/06/1998\|PZ-0033-US,3536,10/05/1998 |
| 972 | rat00020781 | 701464269 | ZS:212821.1 | RALITXT32 | PZ-0108-P,110,02/11/1999 |
| 973 | rat00100296 | 701827434 | ZS:212821.1 | RAKITXT09 | PZ-0132-P,592,08/10/1999 |
| 974 | rat00185004 | 701718036 | ZS:212821.1 | RAKITXT09 | PZ-0132-P,1479,08/10/1999 |
| 975 | rat00024363 | 700435067 | ZS:212821.1 | RAHLNOT02 | PZ-0047-P,124,11/06/1997\|PZ-0047-US,124,11/03/1998 |
| 976 | rat00211395 | 700415992 | ZS:212821.1 | RAHTNOT01 | PZ-0037-US,384,10/06/1998\|PZ-0048-P,384,11/05/1997 |
| 977 | rat00061195 | 700311142 | ZS:212821.1 | RAUCNOT01 | PZ-0029-P,520,09/11/1997\|PZ-0029-US,520,09/04/1998 |
| 978 | rat00003893 | 700812651 | ZS:212821.1 | RAPINOT03 | PZ-0073-P,1226,03/11/1998\|PZ-0073-US,1226,03/09/1999 |
| 979 | rat00184074 | 701021635 | ZS:212821.1 | RAFANOT02 | PZ-0101-P,67,12/10/1998 |
| 980 | rat00028608 | 700519425 | ZS:212821.1 | RABHNOT02 | PZ-0021-US,1575,09/08/1998\|PZ-0050-P,1575,12/11/1997 |
| 981 | rat00050865 | 701489338 | ZS:212821.1 | RALITXT54 | PZ-0135-P,248,08/05/1999 |
| 982 | rat00142197 | 700275567 | ZS:212821.1 | RASINOT02 | PZ-0038-P,498,10/08/1997\|PZ-0038-US,498,10/05/1998 |
| 983 | hu00969530 | 4344032 | LG:401992.11 | SINDNOT01 | |
| 984 | rat00178844 | 700370485 | ZS:271430.1 | RAHYNOT01 | PZ-0033-P,1516,10/08/1997\|PZ-0033-US,1516,10/05/1998 |
| 985 | hu00523841 | 2907890 | IB:293052.1 | THYMNOT05 | PD-0393-P,1543,07/02/1997\|PD-0393-US,1543,06/24/1998 |
| 986 | hu00759605 | 2781237 | IB:293052.1 | OVARTUT03 | PD-0329-P,1864,03/07/1997\|PD-0329-US,1864,03/05/1998 |
| 987 | rat00161197 | 700933680 | ZS:223059.1 | RALINON03 | PZ-0075-P,1418,03/10/1998\|PZ-0075-US,2358,03/09/1999 |
| 988 | rat00214410 | 701714807 | ZS:223059.1 | RABYUNN02 | PZ-0130-P,1205,06/29/1999 |
| 989 | rat00016800 | 700543268 | ZS:223059.1 | RASPNOT01 | PZ-0003-US,2502,05/21/1998\|PZ-0055-P,323,12/12/1997 |
| 990 | rat00113622 | 700629946 | ZS:223059.1 | RATHNOT02 | PZ-0049-US,1640,11/06/1998\|PZ-0072-P,99,03/12/1998 |
| 991 | rat00090205 | 700543958 | ZS:223059.1 | RASPNOT01 | PZ-0003-US,2702,05/21/1998\|PZ-0055-P,523,12/12/1997 |
| 992 | rat00165130 | 700809939 | ZS:223059.1 | RAPINOT03 | PZ-0073-P,395,03/11/1998\|PZ-0073-US,395,03/09/1999 |
| 993 | rat00019997 | 700275945 | ZS:223059.1 | RASINOT02 | PZ-0038-P,657,10/08/1997\|PZ-0038-US,657,10/05/1998 |
| 994 | rat00171632 | 701260994 | ZS:223059.1 | RALINOH03 | PZ-0091-P,3061,10/27/1998 |
| 995 | rat00188498 | 701263538 | ZS:223059.1 | RABXNOT05 | PZ-0090-P,2606,08/12/1998 |
| 996 | rat00044347 | 700499733 | ZS:223059.1 | RAHINOT01 | PZ-0025-US,2145,09/09/1998\|PZ-0044-P,2145,11/04/1997 |
| 997 | rat00012121 | 700363490 | ZS:223059.1 | RALMNON04 | PZ-0017-US,1150,08/05/1998\|PZ-0035-P,1150,10/10/1997 |
| 998 | rat00249136 | 700820713 | ZS:216246.1 | RAESNOT02 | PZ-0032-US,1876,10/07/1998\|PZ-0074-P,639,03/13/1998 |
| 999 | rat00192355 | 700530107 | ZS:216246.1 | RALMNOT01 | PZ-0045-P,1366,11/05/1997\|PZ-0017-US,5163,08/05/1998 |
| 1000 | rat00227393 | 700819863 | ZS:216246.1 | RAESNOT02 | PZ-0032-US,1575,10/07/1998\|PZ-0074-P,338,03/13/1998 |
| 1001 | rat00057555 | 700814470 | ZS:216246.1 | RABFNOT03 | PZ-0063-P,116,02/10/1998\|PZ-0013-US,1209,08/07/1998 |
| 1002 | rat00085573 | 701286468 | ZS:216246.1 | RABXNOT03 | PZ-0087-P,3309,08/11/1998 |
| 1003 | rat00194473 | 700189527 | ZS:216246.1 | RATENOT01 | PZ-0020-P,844,08/06/1997\|PZ-0020-US,844,07/31/1998 |
| 1004 | rat00193088 | 701286761 | ZS:216246.1 | RABXNOT03 | PZ-0087-P,3453,08/11/1998 |
| 1005 | rat00029775 | 700602464 | ZS:216246.1 | RASGNOT02 | PZ-0019-US,1205,08/04/1998\|PZ-0071-P,425,03/11/1998 |
| 1006 | rat00177882 | 700271991 | ZS:216246.1 | RASJNOT01 | PZ-0039-P,358,10/08/1997\|PZ-0039-US,358,10/05/1998 |
| 1007 | rat00219996 | 700856846 | ZS:216246.1 | RAXSNOT02 | PZ-0100-P,1980,11/12/1998 |
| 1008 | rat00233601 | 701910492 | ZS:216246.1 | RABYUNN02 | PZ-0130-P,678,06/29/1999 |
| 1009 | rat00023406 | 700591303 | ZS:216246.1 | RATRNOT04 | PZ-0062-P,572,01/16/1998 |
| 1010 | rat00111931 | 701295547 | ZS:216246.1 | RABXNOT05 | PZ-0090-P,2902,08/12/1998 |
| 1011 | rat00107350 | 700251664 | ZS:216246.1 | RABMNOT01 | PZ-0022-US,2440,09/01/1998\|PZ-0022-P,516,09/11/1997 |
| 1012 | rat00142146 | 700061690 | ZS:216246.1 | RAKINOT02 | PZ-0002-P,1185,06/13/1997\|PZ-0002-US,1927,06/09/1998 |
| 1013 | rat00211383 | 700522555 | ZS:216246.1 | RABMNOT01 | PZ-0022-US,388,09/01/1998\|PZ-0051-P,388,12/12/1997 |
| 1014 | rat00044435 | 700312605 | ZS:216246.1 | RAUCNOT01 | PZ-0029-P,1074,09/11/1997\|PZ-0029-US,1074,09/04/1998 |
| 1015 | rat00021863 | 700486975 | ZS:216246.1 | RATENOT01 | PZ-0020-US,2640,07/31/1998\|PZ-0056-P,863,12/11/1997 |
| 1016 | rat00230438 | 701280837 | ZS:216246.1 | RABXNOT02 | PZ-0087-P,685,08/11/1998 |
| 1017 | rat00184305 | 701263345 | ZS:216246.1 | RABXNOT04 | PZ-0090-P,15,08/12/1998 |
| 1018 | rat00160545 | 700130847 | ZS:216246.1 | RABHNOT01 | PZ-0004-P,3855,07/09/1997\|PZ-0004-US,3855,07/09/1998 |
| 1019 | rat00225625 | 701294142 | ZS:216246.1 | RABXNOT04 | PZ-0090-P,2080,08/12/1998 |
| 1020 | rat00052754 | 700127864 | ZS:216246.1 | RABHNOT01 | PZ-0004-P,2606,07/09/1997\|PZ-0004-US,2606,07/09/1998 |
| 1021 | rat00125754 | 700419566 | ZS:216246.1 | RAHANOT01 | PZ-0040-US,287,10/08/1998\|PZ-0041-P,287,10/09/1997 |
| 1022 | rat00216555 | 700298707 | ZS:216246.1 | RAEONOT02 | PZ-0024-P,131,09/09/1997\|PZ-0024-US,2045,09/10/1998 |
| 1023 | rat00249461 | 700521683 | ZS:216246.1 | RABMNOT01 | PZ-0022-US,65,09/01/1998\|PZ-0051-P,65,12/12/1997 |
| 1024 | rat00085625 | 700489028 | ZS:216246.1 | RATENOT01 | PZ-0056-P,1684,12/11/1997\|PZ-0020-US,3461,07/31/1998 |
| 1025 | rat00138547 | 700821256 | ZS:216246.1 | RAESNOT02 | PZ-0032-US,2078,10/07/1998\|PZ-0074-P,841,03/13/1998 |
| 1026 | rat00166582 | 700297908 | ZS:216246.1 | RAEGNOT01 | PZ-0023-P,811,09/11/1997\|PZ-0023-US,811,09/01/1998 |
| 1027 | rat00245747 | 700129307 | ZS:216246.1 | RABHNOT01 | PZ-0004-P,3206,07/09/1997\|PZ-0004-US,3206,07/09/1998 |
| 1028 | rat00177618 | 700417150 | ZS:216246.1 | RAHTNOT01 | PZ-0037-US,790,10/06/1998\|PZ-0048-P,790,11/05/1997 |
| 1029 | rat00249944 | 700486715 | ZS:216246.1 | RATENOT01 | PZ-0020-US,2506,07/31/1998\|PZ-0056-P,729,12/11/1997 |
| 1030 | rat00000920 | 700268744 | ZS:216246.1 | RAADNOT03 | PZ-0011-US,1461,07/30/1998\|PZ-0031-P,268,10/10/1997 |
| 1031 | rat00121523 | 700174616 | ZS:216246.1 | RATENOT01 | PZ-0020-P,62,08/06/1997\|PZ-0020-US,62,07/31/1998 |
| 1032 | rat00181521 | 700489346 | ZS:216246.1 | RATENOT01 | PZ-0056-P,1793,12/11/1997\|PZ-0020-US,3570,07/31/1998 |
| 1033 | rat00214904 | 701270561 | ZS:216246.1 | RABPFET01 | PZ-0093-P,2550,09/08/1998 |
| 1034 | rat00052481 | 701300219 | ZS:216246.1 | RABXNOT05 | PZ-0090-P,5180,08/12/1998 |
| 1035 | rat00134751 | 700522758 | ZS:216246.1 | RABMNOT01 | PZ-0022-US,476,09/01/1998\|PZ-0051-P,476,12/12/1997 |
| 1036 | rat00022566 | 700068332 | ZS:216246.1 | RABTNOT01 | PZ-0001-P,1566,06/12/1997\|PZ-0001-US,1566,06/08/1998 |
| 1037 | rat00040368 | 700525582 | ZS:216246.1 | RABMNOT01 | PZ-0022-US,1463,09/01/1998\|PZ-0051-P,1463,12/12-1997 |
| 1038 | rat00008580 | 700300221 | ZS:216246.1 | RAEONOT02 | PZ-0024-US,2673,09/10/1998\|PZ-0024-P,759,09/09/1997 |
| 1039 | rat00059250 | 700533847 | ZS:216246.1 | RAEONOT02 | PZ-0024-US,682,09/10/1998\|PZ-0052-P,682,12/11/1997 |
| 1040 | rat00191560 | 701273613 | ZS:216246.1 | RABXNOT01 | PZ-0106-P,1111,01/11/1999 |
| 1041 | rat00017815 | 701255660 | ZS:216246.1 | RALITXT03 | PZ-0096-P,115,10/09/1998 |
| 1042 | hu00042860 | 2747183 | IB:034727.1 | LUNGTUT11 | PD-0326-US,2425,03/04/1997\|P0-0326-P,858,03/07/1997 |
| 1043 | hu00298010 | 996393 | IB:034727.1 | KIDNTUT01 | PD-0137-P,740,03/25/1996\|PD-0137-US,740,03/25/1997 |
| 1044 | hu00933420 | 1447832 | IB:034727.1 | PLACNOT02 | PD-0167-US,2113,05/29/1997\|PD-0193-P,753,06/27/1996 |
| 1045 | hu00667284 | 286060 | IB:034727.1 | EOSIHET02 | PD-0036-US,332,04/26/1995 |
| 1046 | hu00372039 | 3980387 | IB:244682.1 | LUNGTUT08 | PD-0326-US,4258,03/04/1997 |
| 1047 | rat00124233 | 700250549 | ZS:290707.1 | RABHNOT02 | PZ-0021-P,1700,09/09/1997\|PZ-0021-US,4276,09/08/1998 |
| 1048 | rat00239898 | 700123628 | ZS:290707.1 | RABHNOT01 | PZ-0004-P,757,07/09/1997\|PZ-0004-US,757,07/09/1998 |
| 1049 | hu00551266 | 2938247 | IB:334198.1 | THYMFET02 | PD-0361-P,1263,04/24/1997\|PD-0361-US,1263,04/23/1998 |
| 1050 | hu00666846 | 22377 | IB:334198.2 | ADENINB01 | PD-0005-US,1175,02/04/1994\|PD-0033-US,1705,03/29/1995\|PD- |
| | | | | | 0033-1-US,2095,07/13/1995 |
| 1051 | hu00528877 | 1925075 | IB:334198.2 | BRSTNOT02 | PD-0240-P,195,09/30/1996\|PD-0240-US,195,09/29/1997 |
| 1052 | hu00815978 | 1364475 | IB:334198.2 | SCORNON02 | PD-0161-P,25,04/29/1996\|PD-0161-US,25,04/29/1997 |
| 1053 | hu00638589 | 3438723 | IB:334198.2 | PENCNOT06 | PD-0389-P,2229,07/02/1997\|PD-0389-US,2229,06/24/1998 |
| 1054 | hu00031025 | 2303842 | IB:334198.2 | BRSTNOT05 | PD-0292-P,3843,12/19/1996\|PD-0292-US,3843,12/18/1997 |
| 1055 | hu00499778 | 691479 | IB:334198.2 | LUNGTUT02 | PD-0097-P,998,12/20/1995\|PD-0097-US,998,12/20/1996 |
| 1056 | hu00678989 | 2097617 | IB:334198.2 | BRAITUT02 | PD-0111-1-US,4284,12/05/1997\|PD-0275-P,4744,12/06/1996 |
| 1057 | hu00016195 | 680352 | IB:334198.2 | UTRSNOT02 | PD-0095-P,703,12/29/1995\|PD-0095-US,703,12/23/1996 |
| 1058 | hu00442156 | 996586 | IB:334198.2 | KIDNTUT01 | PD-0137-P,832,03/25/1996\|PD-0137-US,832,03/25/1997 |
| 1059 | hu00952341 | 1476048 | IB:334198.2 | LUNGTUT03 | PD-0189-US,1210,06/19/1997\|PD-0189-P,1230,06/20/1996 |
| 1060 | hu00905337 | 2153735 | IB:334198.2 | BRAINOT09 | PD-0275-P,1772,12/06/1996\|PD-0163-US,3806,05/20/1997 |
| 1061 | hu00839062 | 2397557 | IB:334198.2 | THP1AZT01 | PD-0296-P,1081,12/17/1996\|PD-0236-US,1978,08/29/1997 |
| 1062 | hu00804097 | 2171748 | IB:334198.2 | ENDCNOT03 | PD-0279-P,1165,11/22/1996\|PD-0279-US,1165,11/07/1997 |
| 1063 | hu00867242 | 3929904 | IB:334198.2 | KIDNNOT19 | |
| 1064 | hu00346764 | 2734931 | IB:334198.2 | OVARNOT09 | PD-0311-P,107,01/27/1997\|PD-0311-US,107,01/23/1998 |
| 1065 | hu00733855 | 2582411 | IB:334198.2 | KIDNTUT13 | PD-0325-P,4047,03/05/1997 |
| 1066 | hu00505777 | 3089081 | IB:334198.2 | HEAONOT03 | PD-0352-P,1715,04/18/1997\|PD-0352-US,1715,04/24/1998 |
| 1067 | hu00439947 | 663716 | IB:334198.2 | SCORNOT01 | PD-0094-P,153,12/14/1995\|PD-0094-US,153,12/12/1996 |
| 1068 | hu00898254 | 2278413 | IB:334198.2 | PROSNON01 | PD-0198-US,3516,07/28/1997\|PD-0284-P,3516,12/05/1996 |
| 1069 | hu00043131 | 24810 | IB:334198.2 | ADENINB01 | PD-0033-US,2391,03/29/1995\|PD-0005-US,2693,02/04/1994\|PD- |
| | | | | | 0033-1-US,2770,07/13/1995 |
| 1070 | hu00141506 | 2591180 | IB:334198.2 | LUNGNOT22 | PD-0328-P,1022,03/06/1997\|PD-0328-US,1022,03/03/1998 |
| 1071 | hu00561303 | 4907489 | IB:334198.2 | TLYMNOT08 | PS-0007-P,1321,04/15/1999\|PS-0007-US,1321,04/15/1999 |
| 1072 | hu00046521 | 259698 | IB:334198.2 | HNT2RAT01 | PD-0039-P,964,06/30/1995 |
| 1073 | hu00159103 | 21859 | IB:334198.2 | ADENINB01 | PD-0033-US,1553,03/29/1995\|PD-0033-1-US,1945,07/13/1995\|PD- |
| | | | | | 0005-US,859,02/04/1994 |
| 1074 | hu00313147 | 2618859 | IB:334198.2 | GBLANOT01 | PD-0338-P,2065,03/14/1997\|PD-0338-US,2065,03/12/1998 |
| 1075 | hu00901261 | 1450971 | IB:334198.2 | PENITUT01 | PD-0192-P,236,06/27/1996 |
| 1076 | hu00627230 | 3079454 | IB:334198.2 | BRAIUNT01 | PD-0348-P,422,03/31/1997\|PD-0348-US,422,03/30/1998 |
| 1077 | hu00203835 | 1493650 | IB:334198.2 | PROSNON01 | PD-0198-US,5231,07/28/1997\|PD-0198-P,624,07/29/1996 |
| 1078 | hu00386214 | 2370759 | IB:334198.2 | ADRENOT07 | PD-0301-P,2613,12/19/1996\|PD-0301-US,2613,12/17/1997 |
| 1079 | hu00923443 | 2290353 | IB:334198.2 | BRAINON01 | PD-0274-P,3555,11/20/1996 |
| 1080 | hu00726614 | 2275525 | IB:334198.2 | PROSNON01 | PD-0198-US,2265,07/28/1997\|PD-0284-P,2265,12/05/1996 |
| 1081 | hu00324675 | 2287631 | IB:334198.2 | BRAINON01 | PD-0274-P,2022,11/20/1996 |
| 1082 | hu00616149 | 2986658 | IB:334198.2 | CARGDIT01 | PD-0349-P,710,03/31/1997\|PD-0349-US,710,03/31/1998 |
| 1083 | hu00823419 | 2452912 | IB:334198.2 | ENDANOT01 | PD-0299-P,1111,12/16/1996\|PD-0299-US,1111,12/04/1997 |
| 1084 | hu00367308 | 2509602 | IB:334198.2 | CONUTUT01 | PD-0300-P,2985,12/20/1996\|PD-0300-US,2985,12/18/1997 |
| 1085 | hu00554095 | 3467240 | IB:334198.2 | BRAIDIT01 | PD-0384-P,442,06/06/1997\|PD-0384-US,442,06/04/1998 |
| 1086 | hu00647155 | 3079537 | IB:334198.2 | BRAIUNT01 | PD-0348-P,453,03/31/1997\|PD-0348-US,453,03/30/1998 |
| 1087 | hu00761366 | 1271327 | IB:334198.2 | TESTTUT02 | PD-0164-P,87,05/16/1996\|PD-0164-US,87,05/15/1997 |
| 1088 | hu00783265 | 452363 | IB:334198.2 | TLYMNOT02 | PD-0073-P,1768,10/02/1995 |
| 1089 | hu00540697 | 3029510 | IB:334198.2 | HEARFET02 | PD-0353-P,1392,04/18/1997\|PD-0353-US,1392,03/17/1998 |
| 1090 | hu00881555 | 2838736 | IB:334198.2 | DRGLNOT01 | PD-0337-P,585,03/18/1997\|PD-0337-US,585,03/05/1998 |
| 1091 | hu00457495 | 2503774 | IB:334198.2 | CONUTUT01 | PD-0300-P,687,12/20/1996\|PD-0300-US,687,12/18/1997 |
| 1092 | hu00364798 | 683266 | IB:334198.2 | UTRSNOT02 | PD-0095-P,1448,12/29/1995\|PD-0095-US,1448,12/23/1996 |
| 1093 | hu00110621 | 679322 | IB:334198.2 | UTRSNOT02 | PD-0095-P,341,12/29/1995\|PD-0095-US,341,12/23/1996 |
| 1094 | hu00277043 | 2941393 | IB:334198.2 | HEAONOT03 | PD-0352-P,1913,04/18/1997\|PD-0352-US,1913,04/24/1998 |
| 1095 | hu00201587 | 1517789 | IB:334198.2 | PANCTUT01 | PD-0196-P,1634,06/21/1996 |
| 1096 | hu00320966 | 217 | IB:334198.2 | U937NOT01 | PD-0001-3-US,165,02/14/1994\|PD-0001-US,165,07/17/1992\|PD- |
| | | | | | 0001-1-US,183,11/19/1992 |
| 1097 | hu00693044 | 2697521 | IB:334198.2 | UTRSNOT12 | PD-0346-P,1411,03/27/1997\|PD-0346-US,5696,03/27/1998 |
| 1098 | hu00634454 | 3279421 | IB:334198.2 | STOMFET02 | PD-0379-P,969,06/02/1997\|PD-0379-US,969,05/15/1998 |
| 1099 | hu00711495 | 54555 | IB:334198.2 | | PD-0008-US,1131,06/27/1994 |
| 1100 | hu00610579 | 2742812 | IB:334198.2 | BRSTTUT14 | PD-0320-P,3396,02/13/1997\|PD-0320-US,3396,02/11/1998 |
| 1101 | hu00340966 | 2583990 | IB:334198.2 | BRAITUT22 | PD-0304-P,3075,01/28/1997\|PD-0304-US,3075,01/28/1998 |
| 1102 | hu00447662 | 2349158 | IB:334198.2 | COLSUCT01 | PD-0278-P,320,12/05/1996\|PD-0278-US,320,11/21/1997 |
| 1103 | hu00267528 | 3567447 | IB:334198.2 | BRONNOT02 | PS-0003-P,1248,06/06/1997\|PS-0003-US,1248,06/06/1997 |
| 1104 | hu00570983 | 2654142 | IB:334198.2 | THYMNOT04 | PD-0315-P,3877,01/30/1997\|PD-0315-US,3877,01/30/1998 |
| 1105 | hu00589236 | 266368 | IB:334198.2 | HNT2NOT01 | PD-0038-P,114,06/16/1995 |
| 1106 | hu00494959 | 654402 | IB:334198.2 | EOSINOT03 | PR-0002-1-US,1583,11/19/1997\|PR-0002-P,1583,12/13/1995\|PR- |
| | | | | | 0002-US,1583,12/12/1996 |
| 1107 | hu00854000 | 21552 | IB:334198.2 | ADENINB01 | PD-0033-US,1477,03/29/1995\|PD-0033-1-US,1869,07/13/1995\|PD- |
| | | | | | 0005-US,699,02/04/1994 |
| 1108 | hu00155044 | 956044 | IB:334198.2 | KIDNNOT05 | PD-0261-1-US,2524,10/29/1997\|PD-0142-P,785,04/10/1996\|PD- |
| | | | | | 0142-US,785,04/10/1997 |
| 1109 | hu00622162 | 598239 | IB:334198.2 | BRSTNOT02 | PD-0061-P,281,11/15/1995\|PD-0061-US,281,11/15/1996 |
| 1110 | hu00304814 | 2809547 | IB:334198.2 | TLYMNOT04 | PD-0363-P,6237,05/02/1997\|PD-0363-US,6237,04/30/1998 |
| 1111 | hu00008564 | 1475663 | IB:334198.2 | LUNGTUT03 | PD-0189-US,1110,06/19/1997\|PD-0189-P,1130,06/20/1996 |
| 1112 | hu00191328 | 2445935 | IB:334198.2 | THP1NOT03 | PD-0286-P,2356,11/25/1996\|PD-0286-US,2356,11/04/1997 |
| 1113 | hu00652363 | 1513731 | IB:334198.2 | PANCTUT01 | PD-0196-P,666,06/21/1996 |
| 1114 | hu00760337 | 2586284 | IB:334198.2 | BRAITUT22 | PD-0304-P,3982,01/28/1997\|PD-0304-US,3982,01/28/1998 |
| 1115 | hu00482708 | 2699720 | IB:334198.2 | OVARTUT10 | PD-0341-P,2608,03/20/1997\|PD-0341-US,2608,03/19/1998 |
| 1116 | hu00342677 | 479348 | IB:334198.2 | MMLR2DT01 | PD-0044-P,1277,10/02/1995\|PD-0044-US,1277,10/02/1996 |
| 1117 | hu00495256 | 2752584 | IB:334198.2 | THP1AZS08 | PD-0362-P,1596,05/01/1997\|PD-0362-US,1596,04/29/1998 |
| 1118 | hu00264785 | 1850751 | IB:334198.2 | LUNGFET03 | PD-0230-P,928,08/23/1996\|PD-0230-US,928,08/22/1997 |
| 1119 | hu00334698 | 2133163 | IB:334198.2 | ENDCNOT01 | PD-0257-P,154,10/23/1996\|PD-0257-US,154,10/22/1997 |
| 1120 | hu00939481 | 2154178 | IB:334198.2 | BRAINOT09 | PD-0275-P,1927,12/06/1996\|PD-0163-US,3961,05/20/1997 |
| 1121 | rat00219199 | 700179763 | ZS:206489.1 | RALUNOT01 | PZ-0006-P,676,07/10/1997\|PZ-0006-US,676,07/10/1998 |
| 1122 | rat00177802 | 700776044 | ZS:206489.2 | RAPINOT02 | PZ-0065-P,213,02/11/1998\|PZ-0065-US,213,02/10/1999 |
| 1123 | rat00179794 | 700860742 | ZS:206489.2 | RABGNOT02 | PZ-0076-P,1473,04/06/1998\|PZ-0076-US,1473,04/06/1999 |
| 1124 | rat00199742 | 700862233 | ZS:206489.2 | RABGNOT02 | PZ-0076-P,2100,04/06/1998\|PZ-0076-US,2100,04/06/1999 |
| 1125 | hu00370477 | 1700421 | IB:235691.7 | BLADTUT05 | PD-0221-US,1011,07/31/1997\|PD-0202-P,1418,07/31/1996 |
| 1126 | hu00201755 | 2914593 | IB:235691.7 | THYMFET03 | PD-0345-P,252,03/21/1997\|PD-0345-US,252,03/18/1998 |
| 1127 | hu00352946 | 1878873 | IB:235691.7 | LEUKNOT03 | PD-0246-P,2728,10/04/1996\|PD-0246-US,2728,10/02/1997 |
| 1128 | hu00422951 | 1391107 | IB:235691.7 | EOSINOT01 | PR-0009-P,1019,06/04/1996\|PR-0009-US,1019,06/03/1997 |
| 1129 | hu00509064 | 2964175 | IB:235691.7 | SCORNOT04 | PD-0360-P,451,04/24/1997\|PD-0360-US,451,04/23/1998 |
| 1130 | hu01263994 | 6313477 | LG:350527.5 | NERDTDN03 | |
| 1131 | hu01115208 | 6613886 | LG:411605.10 | ARTANOT07 | |
| 1132 | hu01072755 | 6270862 | LG:411605.11 | BRAIFEN03 | |
| 1133 | hu01005858 | 5814585 | LG:411605.11 | PROSTUS23 | |
| 1134 | hu01065170 | 6513219 | LG:411605.11 | THYMDIT01 | |
| 1135 | hu01198782 | 6421324 | LG:411605.11 | BRSTUNT01 | |
| 1136 | hu01148976 | 5864057 | LG:411605.11 | COLTDIT04 | |
| 1137 | hu01163198 | 6401763 | LG:411605.11 | UTRENOT10 | |
| 1138 | hu01281639 | 6378821 | LG:411605.11 | FIBAUNT01 | |
| 1139 | hu01123628 | 6496712 | LG:411605.11 | COLNNOT41 | |
| 1140 | hu01154039 | 6419875 | LG:411605.11 | BRSTUNT01 | |
| 1141 | hu01206351 | 5822327 | LG:411605.11 | PROSTUS23 | |
| 1142 | hu01014732 | 5813672 | LG:411605.11 | PROSTUS23 | |
| 1143 | hu00986147 | 6408660 | LG:411605.12 | UTREDIT10 | |
| 1144 | hu01215972 | 4518370 | LG:411605.12 | SINJNOT03 | |
| 1145 | hu01051075 | 6517553 | LG:411605.12 | BRAFTDT02 | |
| 1146 | hu01218852 | 6136188 | LG:411605.12 | BMARTXT02 | |
| 1147 | hu01158132 | 6410975 | LG:411605.12 | UTREDIT10 | |
| 1148 | hu01011622 | 5823548 | LG:411605.12 | PROSTUS23 | |
| 1149 | hu01339166 | 6369723 | LG:411605.12 | ENDINOT01 | |
| 1150 | hu01034696 | 6014804 | LG:411605.12 | FIBRUNT02 | |
| 1151 | hu01159033 | 6398907 | LG:411605.12 | UTRENOT10 | |
| 1152 | hu00994745 | 6568514 | LG:411605.12 | MCLDTXN05 | |
| 1153 | hu01054653 | 5819602 | LG:411605.12 | PROSTUS23 | |
| 1154 | hu01307184 | 6399030 | LG:411605.12 | UTRENOT10 | |
| 1155 | hu01195431 | 6591075 | LG:411605.12 | TLYMUNT03 | |
| 1156 | hu01302613 | 6257326 | LG:411605.12 | BMARTXT06 | |
| 1157 | hu01191060 | 5698802 | LG:411605.12 | BRSTTUT13 | |
| 1158 | rat00177217 | 701229180 | ZS:221302.1 | RASJNON03 | PZ-0098-P,39,11/12/1998 |
| 1159 | rat00049893 | 700934730 | ZS:221302.1 | RALINON03 | PZ-0075-P,1877,03/10/1998\|PZ-0075-US,2817,03/09/1999 |
| 1160 | rat00187352 | 700948690 | ZS:221302.1 | RASPNON02 | PZ-0099-P,1022,11/10/1998 |
| 1161 | rat00114238 | 700295204 | ZS:221302.1 | RABFNOT02 | PZ-0013-P,1046,08/08/1997\|PZ-0013-US,1046,08/07/1998 |
| 1162 | rat00177786 | 701277731 | ZS:221302.1 | RABFNON02 | PZ-0097-P,1618,11/12/1998 |
| 1163 | rat00136648 | 701334863 | ZS:221302.1 | RALINON08 | PZ-0034-US,2345,10/08/1998 |
| 1164 | rat00046766 | 701274943 | ZS:221302.1 | RABFNON02 | PZ-0097-P,593,11/12/1998 |
| 1165 | rat00200496 | 701275514 | ZS:221302.1 | RABFNON02 | PZ-0097-P,806,11/12/1998 |
| 1166 | rat00100831 | 700127275 | ZS:221302.1 | RABHNOT01 | PZ-0004-P,2355,07/09/1997\|PZ-0004-US,2355,07/09/1998 |
| 1167 | rat00132474 | 701333270 | ZS:221302.1 | RALINON08 | PZ-0034-US,1716,10/08/1998 |
| 1168 | rat00144368 | 701944168 | ZS:211420.2 | RALITXT1K | PZ-0134-P,814,08/12/1999 |
| 1169 | rat00168417 | 701466250 | ZS:211420.2 | RALITXT32 | PZ-0108-P,480,02/11/1999 |
| 1170 | rat00076851 | 701944268 | ZS:211420.2 | RALITXT1K | PZ-0134-P,833,08/12/1999 |
| 1171 | rat00216982 | 701258551 | ZS:211420.2 | RALITXT03 | PZ-0096-P,883,10/09/1998 |
| 1172 | rat00248339 | 701616185 | ZS:211420.2 | RALITXT61 | PZ-0127-P,123,07/07/1999 |
| 1173 | rat00213314 | 700141178 | ZS:211420.2 | RALINOT01 | PZ-0010-P,970,07/10/1997\|PZ-0010-US,970,07/10/1998 |
| 1174 | rat00105136 | 701734388 | ZS:211420.2 | RALITXT1C | PZ-0126-P,351,07/08/1999 |
| 1175 | rat00130615 | 701725735 | ZS:211420.2 | RALITXT1A | PZ-0122-P,9,06/03/1999 |
| 1176 | rat00251888 | 701339880 | ZS:211420.2 | RALITXT08 | PZ-0092-P,1425,08/13/1998 |
| 1177 | rat00221660 | 701475064 | ZS:211420.2 | RALITXT41 | PZ-0123-P,1034,06/04/1999 |
| 1178 | rat00042254 | 700303823 | ZS:211420.2 | RALINOT01 | PZ-0026-P,555,09/12/1997\|PZ-0026-US,555,09/04/1998 |
| 1179 | rat00010468 | 700524067 | ZS:211420.2 | RABMNOT01 | PZ-0022-US,984,09/01/1998\|PZ-0051-P,984,12/12/1997 |
| 1180 | rat00120763 | 701880885 | ZS:211420.2 | RALITXS02 | PZ-0133-P,1322,08/10/1999 |
| 1181 | rat00028407 | 701396970 | ZS:211420.2 | RALITXT14 | PZ-0094-P,3057,09/11/1998 |
| 1182 | rat00107373 | 700132732 | ZS:211420.2 | RALMNOT01 | PZ-0017-US,1998,08/05/1998\|PZ-0017-P,384,08/06/1997 |
| 1183 | rat00121848 | 700140788 | ZS:211420.2 | RALINOT01 | PZ-0010-P,885,07/10/1997\|PZ-0010-US,885,07/10/1998 |
| 1184 | hu00475533 | 2234427 | IB:012835.1 | PANCTUT02 | PD-0295-P,201,12/20/1996\|PD-0191-US,2359,06/19/1997 |
| 1185 | hu00764500 | 2242421 | IB:012835.1 | PANCTUT02 | PD-0295-P,2612,12/20/1996\|PD-0191-US,4770,06/19/1997 |
| 1186 | hu00589051 | 2703885 | IB:405464.1 | OVARTUT10 | PD-0341-P,4112,03/20/1997\|PD-0341-US,4112,03/19/1998 |
| 1187 | hu00518649 | 3328680 | IB:405464.1 | HEAONOT04 | PD-0387-P,3175,07/03/1997\|PD-0387-US,3175,06/30/1998 |
| 1188 | hu00267484 | 389504 | IB:017960.1 | THYMNOT02 | PD-0041-P,3211,09/26/1995\|PD-0077-P,3211,10/02/1995\|PD-0077- |
| | | | | | US,3211,10/02/1996 |
| 1189 | hu00467745 | 343775 | IB:017960.1 | THYMNOT02 | PD-0041-P,253,09/26/1995\|PD-0077-P,253,10/02/1995\|PD-0077- |
| | | | | | US,253,10/02/1996 |
| 1190 | hu00866865 | 2749717 | IB:197797.10 | LUNGTUT11 | PD-0326-P,1699,03/07/1997\|PD-0326-US,3266,03/04/1997 |
| 1191 | hu00679638 | 2795118 | IB:197797.10 | NPOLNOT01 | PD-0340-P,330,03/28/1997\|PD-0340-US,330,03/27/1998 |
| 1192 | hu00000052 | 2544029 | IB:197797.10 | UTRSNOT11 | PD-0316-P,2400,01/29/1997\|PD-0316-US,2400,01/26/1998 |
| 1193 | hu00679918 | 1488916 | IB:197797.10 | UCMCL5T01 | PD-0194-P,1896,06/28/1996\|PD-0194-US,1896,06/27/1997\|PD-0184 |
| | | | | | P,567,06/24/1996 |
| 1194 | hu00221048 | 2482284 | IB:197797.10 | SMCANOT01 | PD-0298-P,2062,01/30/1998\|PD-0298-US,2062,01/30/1998 |
| 1195 | hu00333450 | 1591308 | IB:197797.10 | CARGNOT01 | PR-0010-P,294,06/27/1996\|PR-0010-US,294,06/26/1997 |
| 1196 | hu00236251 | 2748126 | IB:197797.10 | LUNGTUT11 | PD-0326-P,1175,03/07/1997\|PD-0326-US,2742,03/04/1997 |
| 1197 | hu00013962 | 2477774 | IB:197797.10 | SMCANOT01 | PD-0298-P,845,01/30/1998\|PD-0298-US,845,01/30/1998 |
| 1198 | hu00077601 | 2542849 | IB:197797.10 | UTRSNOT11 | PD-0316-P,2002,01/29/1997\|PD-0316-US,2002,01/26/1998 |
| 1199 | hu00342680 | 3973829 | IB:197797.10 | ADRETUT06 | PD-0333-US,3227,03/12/1998 |
| 1200 | hu00475701 | 1699889 | IB:197797.10 | BLADTUT05 | PD-0202-P,1208,07/31/1996\|PD-0221-US,801,07/31/1997 |
| 1201 | hu00295127 | 1475835 | IB:197797.10 | LUNGTUT03 | PD-0189-US,1161,06/19/1997\|PD-0189-P,1181,06/20/1996 |
| 1202 | hu00005869 | 4346925 | IB:197797.10 | TLYMTXT01 | PS-0008-P,487,04/16/1998\|PS-0008-US,487,04/16/1999 |
| 1203 | hu00918443 | 854372 | IB:197797.10 | NGANNOT01 | PD-0123-US,954,02/28/1997\|PD-0125-P,954,03/07/1996 |
| 1204 | hu00750908 | 1489193 | IB:197797.10 | UCMCL5T01 | PD-0194-P,1955,06/28/1996\|PD-0194-US,1955,06/27/1997\|PD-0184 |
| | | | | | P,627,06/24/1996 |
| 1205 | hu00735159 | 813961 | IB:197797.10 | OVARTUT01 | PD-0112-1-US,119,11/14/1997\|PD-0112-P,119,02/22/1996\|PD-0112- |
| | | | | | US,119,02/21/1997 |
| 1206 | hu00349275 | 676879 | IB:197797.10 | CRBLNOT01 | PD-0093-P,1318,12/18/1995\|PD-0089-US,3014,11/27/1996 |
| 1207 | hu00757851 | 2883157 | IB:197797.10 | UTRSTUT05 | PD-0346-US,2373,03/27/1998\|PD-0346-P,4421,03/27/1997 |
| 1208 | hu00645964 | 1930333 | IB:197797.10 | COLNTUT03 | PD-0242-P,1059,09/25/1996\|PD-0174-US,2729,05/13/1997 |
| 1209 | hu00599372 | 2465467 | IB:197797.10 | THYRNOT08 | PD-0394-P,2053,07/02/1997\|PD-0394-US,2053,06/24/1998 |
| 1210 | hu00466677 | 3979618 | IB:197797.10 | LUNGTUT08 | PD-0326-US,4026,03/04/1997 |
| 1211 | hu00871867 | 1548750 | IB:197797.10 | PROSNOT06 | PD-0199-P,4543,07/30/1996 |
| 1212 | hu00669884 | 2482006 | IB:197797.10 | SMCANOT01 | PD-0298-P,1980,01/30/1998\|PD-0298-US,1980,01/30/1998 |
| 1213 | hu00032005 | 3972034 | IB:197797.10 | ADRETUT06 | PD-0333-US,2450,03/12/1998 |
| 1214 | hu00406042 | 1302173 | IB:197797.10 | PLACNOT02 | PD-0167-P,1202,05/30/1996\|PD-0167-US,1202,05/29/1997 |
| 1215 | hu00023490 | 1754053 | IB:197797.10 | LIVRTUT01 | PD-0220-P,1136,07/30/1996 |
| 1216 | hu00537201 | 1805622 | IB:197797.10 | SINTNOT13 | PD-0234-P,713,08/28/1996 |
| 1217 | hu00413338 | 355076 | IB:197797.10 | RATRNOT01 | PD-0053-P,5220,10/10/1995\|PD-0053-US,5220,10/09/1996 |
| 1218 | hu00086921 | 1445360 | IB:197797.10 | PLACNOT02 | PD-0193-P,109,06/27/1996\|PD-0167-US,1469,05/29/1997 |
| 1219 | hu00822446 | 1915970 | IB:197797.10 | PROSTUT04 | PD-0232-P,1990,09/05/1996\|PD-0197-US,4558,06/23/1997 |
| 1220 | hu00369125 | 491397 | IB:197797.10 | HNT2AGT01 | |
| 1221 | hu00149385 | 1405153 | IB:197797.10 | LATRTUT02 | PD-0187-P,1137,06/25/1996\|PD-0176-US,3031,05/20/1997 |
| 1222 | hu00088909 | 1423720 | IB:197797.10 | CARGDIT02 | PR-0008-P,1050,06/04/1996\|PR-0008-US,1050,06/03/1997 |
| 1223 | hu00289537 | 2875708 | IB:197797.10 | THYRNOT10 | PD-0395-P,3012,07/02/1997\|PD-0395-US,3012,06/23/1998 |
| 1224 | hu00465993 | 550170 | IB:197797.10 | BEPINOT01 | PD-0056-P,1116,10/31/1995\|PD-0056-US,1116,10/16/1996 |
| 1225 | hu00709688 | 2278238 | IB:197797.10 | PROSNON01 | PD-0198-US,3436,07/28/1997\|PD-0284-P,3436,12/05/1996 |
| 1226 | hu00785849 | 1332454 | IB:197797.10 | PANCNOT07 | PD-0172-P,1238,05/23/1996\|PD-0172-US,1238,05/22/1997 |
| 1227 | hu00071401 | 155152 | IB:197797.10 | THP1PLB02 | |
| 1228 | hu00133016 | 1332142 | IB:197797.10 | PANCNOT07 | PD-0172-P,1140,05/23/1996\|PD-0172-US,1140,05/22/1997 |
| 1229 | hu00712341 | 2683259 | IB:197797.10 | SINIUCT01 | PD-0330-P,1434,02/12/1997\|PD-0330-US,1434,02/10/1998 |
| 1230 | hu00651621 | 2827925 | IB:197797.10 | TLYMNOT03 | PD-0363-P,428,05/02/1997\|PD-0363-US,428,04/30/1998 |
| 1231 | hu00801723 | 2862812 | IB:197797.10 | SININOT03 | PD-0343-P,1492,03/25/1997\|PD-0343-US,1492,03/25/1998 |
| 1232 | hu00325174 | 2122664 | IB:197797.10 | BRSTNOT07 | PD-0166-US,828,05/13/1997\|PD-0276-P,828,11/21/1996 |
| 1233 | hu00123496 | 2554840 | IB:197797.10 | THYMNOT03 | PD-0315-P,1004,01/30/1997\|PD-0315-US,1004,01/30/1998 |
| 1234 | hu00644372 | 2630572 | IB:197797.10 | COLNTUT15 | PD-0305-P,282,01/22/1997\|PD-0305-US,282,01/22/1998 |
| 1235 | hu00452495 | 465062 | IB:197797.10 | LATRNOT01 | PD-0053-P,1717,10/10/1995\|PD-0053-US,1717,10/09/1996 |
| 1236 | hu00667723 | 2908988 | IB:197797.10 | THYMNOT05 | PD-0393-P,2027,07/02/1997\|PD-0393-US,2027,06/24/1998 |
| 1237 | hu00774680 | 1870177 | IB:197797.10 | SKINBIT01 | PD-0253-P,1236,10/03/1996\|PD-0253-US,1236,09/30/1997 |
| 1238 | hu00908188 | 3340773 | IB:197797.10 | SPLNNOT10 | PD-0378-P,3069,06/05/1997\|PD-0378-US,3069,05/19/1998 |
| 1239 | hu00896372 | 938004 | IB:197797.10 | CERVNOT01 | PD-0128-P,2451,03/22/1996 |
| 1240 | hu00095768 | 2848865 | IB:197797.10 | BRSTTUT13 | PD-0336-P,362,03/18/1997 |
| 1241 | hu00083110 | 2439949 | IB:197797.10 | EOSITXT01 | PR-0013-P,1235,11/25/1996\|PR-0011-US,2591,08/29/1997 |
| 1242 | hu00241234 | 1414913 | IB:197797.10 | BRAINOT12 | PD-0159-P,922,06/20/1996\|PD-0159-US,922,06/18/1997 |
| 1243 | hu00305063 | 2720175 | IB:197797.10 | LUNGTUT10 | PD-0327-P,247,03/05/1997\|PD-0327-US,247,03/03/1998 |
| 1244 | hu00377000 | 2685714 | IB:197797.10 | LUNGNOT23 | PD-0388-P,609,07/03/1997\|PD-0388-US,609,06/19/1998 |
| 1245 | hu00282403 | 2371533 | IB:197797.10 | ADRENOT07 | PD-0301-P,2911,12/19/1996\|PD-0301-US,2911,12/17/1997 |
| 1246 | hu00003638 | 3088650 | IB:197797.10 | HEAONOT03 | PD-0352-P,1556,04/18/1997\|PD-0352-US,1556,04/24/1998 |
| 1247 | hu00524118 | 2152591 | IB:197797.10 | BRAINOT09 | PD-0275-P,1359,12/06/1996\|PD-0163-US,3393,05/20/1997 |
| 1248 | hu00307641 | 780556 | IB:197797.10 | MYOMNOT01 | PD-0113-P,437,02/27/1996\|PD-0113-US,437,02/27/1997 |
| 1249 | hu00040648 | 3947580 | IB:197797.10 | DRGCNOT01 | PD-0337-US,6903,03/05/1998 |
| 1250 | hu00116735 | 3981972 | IB:197797.10 | LUNGTUT08 | PD-0326-US,4719,03/04/1997 |
| 1251 | hu00344058 | 2659340 | IB:197797.10 | LUNGTUT09 | PD-0310-P,3309,01/28/1997\|PD-0310-US,3309,01/28/1998 |
| 1252 | hu00060966 | 937943 | IB:197797.10 | CERVNOT01 | PD-0128-P,2427,03/22/1996 |
| 1253 | hu00458246 | 1320953 | IB:197797.10 | BLADNOT04 | PD-0202-US,878,07/31/1997\|PD-0170-P,882,05/29/1996 |
| 1254 | hu00298623 | 3972523 | IB:197797.10 | ADRETUT06 | PD-0333-US,2675,03/12/1998 |
| 1255 | hu00052869 | 1871348 | IB:197797.10 | SKINBIT01 | PD-0253-P,1636,10/03/1996\|PD-0253-US,1636,09/30/1997 |
| 1256 | hu00530798 | 2660893 | IB:197797.10 | LUNGTUT09 | PD-0310-P,3914,01/28/1997\|PD-0310-US,3914,01/28/1998 |
| 1257 | hu00961646 | 2665670 | IB:197797.10 | ADRENOT08 | PD-0317-P,1773,02/11/1997\|PD-0317-US,1773,02/10/1998 |
| 1258 | hu00948713 | 3180353 | IB:197797.10 | TLYJNOT01 | PD-0382-P,642,06/06/1997\|PD-0382-US,642,05/21/1998 |
| 1259 | hu00547548 | 1484296 | IB:197797.10 | CORPNOT02 | PD-0185-P,2878,06/26/1996\|PD-0185-US,2878,06/26/1997 |
| 1260 | rat00151369 | 701607583 | ZS:222773.1 | RALITXT09 | PZ-0116-P,773,04/29/1999 |
| 1261 | rat00127186 | 700372543 | ZS:222773.1 | RARANOT01 | PZ-0037-US,1804,10/06/1998\|PZ-0037-P,615,10/09/1997 |
| 1262 | rat00069932 | 700781722 | ZS:222773.1 | RABSNOT02 | PZ-0057-P,547,01/16/1998\|PZ-0057-US,547,01/11/1999 |
| 1263 | rat00192972 | 701649506 | ZS:222773.1 | RALITXT10 | PZ-0117-P,57,05/07/1999 |
| 1264 | rat00035697 | 700768474 | ZS:222773.1 | RAHYNOT02 | PZ-0078-P,1452,04/06/1998\|PZ-0033-US,3116,10/05/1998 |
| 1265 | rat00026987 | 700366914 | ZS:222773.1 | RAMGNOT01 | PZ-0046-P,622,11/04/1997\|PZ-0046-US,622,11/03/1998 |
| 1266 | rat00163788 | 700274636 | ZS:222773.1 | RASINOT02 | PZ-0038-P,145,10/08/1997\|PZ-0038-US,145,10/05/1998 |
| 1267 | rat00168756 | 700328048 | ZS:222773.1 | RASNNOT01 | PZ-0095-P,1168,09/09/1998\|PZ-0054-US,3550,12/10/1998 |
| 1268 | rat00000372 | 700485657 | ZS:222773.1 | RATENOT01 | PZ-0020-US,2023,07/31/1998\|PZ-0056-P,246,12/11/1997 |
| 1269 | rat00202332 | 700635667 | ZS:222773.1 | RATONOT01 | PZ-0036-US,240,10/09/1998 |
| 1270 | rat00110821 | 701232896 | ZS:222773.1 | RASJNON03 | PZ-0098-P,2108,11/12/1998 |
| 1271 | rat00010175 | 701580093 | ZS:222773.1 | RALIUNT03 | PZ-0121-P,245,05/03/1999 |
| 1272 | rat00190601 | 700519840 | ZS:222773.1 | RABHNOT02 | PZ-0021-US,1786,09/08/1998\|PZ-0050-P,1786,12/11/1997 |
| 1273 | rat00085202 | 700627492 | ZS:222773.1 | RAFBNOT01 | PZ-0064-P,951,02/12/1998\|PZ-0064-US,951,02/10/1999 |
| 1274 | rat00165071 | 700530153 | ZS:222773.1 | RALMNOT01 | PZ-0045-P,1389,11/05/1997\|PZ-0017-US,5186,08/05/1998 |
| 1275 | rat00035607 | 701419250 | ZS:222773.1 | RASLNOT01 | PZ-0107-P,648,01/14/1999 |
| 1276 | rat00162742 | 700636665 | ZS:222773.1 | RATONOT01 | PZ-0036-US,553,10/09/1998 |
| 1277 | rat00080178 | 701343275 | ZS:222773.1 | RALITXT11 | PZ-0094-P,888,09/11/1998 |
| 1278 | rat00206754 | 701319684 | ZS:222773.1 | RALITXT05 | PZ-0092-P,437,08/13/1998 |
| 1279 | rat00244717 | 700766587 | ZS:222773.1 | RAHYNOT02 | PZ-0033-US,2350,10/05/1998\|PZ-0078-P,686,04/06/1998 |
| 1280 | rat00078375 | 701392692 | ZS:222773.1 | RALITXT14 | PZ-0094-P,2297,09/11/1998 |
| 1281 | rat00218796 | 700785820 | ZS:222773.1 | RABSNOT02 | PZ-0057-P,2321,01/16/1998\|PZ-0057-US,2321,01/11/1999 |
| 1282 | rat00112251 | 700914592 | ZS:222773.1 | RAXSNOT02 | PZ-0100-P,3656,11/12/1998 |
| 1283 | rat00204007 | 700625377 | ZS:222773.1 | RAFBNOT01 | PZ-0064-P,303,02/12/1998\|PZ-0064-US,303,02/10/1999 |
| 1284 | rat00171140 | 700785521 | ZS:222773.1 | RABSNOT02 | PZ-0057-P,2188,01/16/1998\|PZ-0057-US,2188,01/11/1999 |
| 1285 | rat00131225 | 700306832 | ZS:222773.1 | RASDNOT02 | PZ-0027-US,1332,09/08/1998\|PZ-0028-P,78,09/08/1997 |
| 1286 | rat00125440 | 700626941 | ZS:222773.1 | RAFBNOT01 | PZ-0064-P,762,02/12/1998\|PZ-0064-US,762,02/10/1999 |
| 1287 | rat00170710 | 700276565 | ZS:222773.1 | RASINOT02 | PZ-0038-P,855,10/08/1997\|PZ-0038-US,855,10/05/1998 |
| 1288 | rat00125900 | 700314659 | ZS:222773.1 | RAESNOT01 | PZ-0032-P,529,10/08/1997\|PZ-0032-US,529,10/07/1998 |
| 1289 | rat00207810 | 700420110 | ZS:222773.1 | RAHANOT01 | PZ-0040-US,462,10/08/1998\|PZ-0041-P,462,10/09/1997 |
| 1290 | rat00114355 | 700246776 | ZS:222773.1 | RABCNOT01 | PZ-0012-US,1145,08/04/1998 |
| 1291 | rat00252245 | 700288432 | ZS:222773.1 | RASDNOT01 | PZ-0027-P,931,09/08/1997\|PZ-0027-US,931,09/08/1999 |
| 1292 | rat00059151 | 701519417 | ZS:222773.1 | RALITXT62 | PZ-0136-P,402,08/06/1999 |
| 1293 | rat00095233 | 700629369 | ZS:222773.1 | RAFBNOT01 | PZ-0064-P,1537,02/12/1998\|PZ-0064-US,1537,02/10/1999 |
| 1294 | rat00024880 | 701025922 | ZS:222773.1 | RAFANOT02 | PZ-0101-P,924,12/10/1998 |
| 1295 | rat00036707 | 700631958 | ZS:222773.1 | RATHNOT02 | PZ-0049-US,2279,11/06/1998\|PZ-0072-P,738,03/12/1998 |
| 1296 | rat00227417 | 700636674 | ZS:222773.1 | RATONOT01 | PZ-0036-US,558,10/09/1998 |
| 1297 | rat00150890 | 700914122 | ZS:222773.1 | RAXSNOT02 | PZ-0100-P,3482,11/12/1998 |
| 1298 | rat00252960 | 701024824 | ZS:222773.1 | RAFANOT02 | PZ-0101-P,839,12/10/1998 |
| 1299 | rat00038841 | 700910639 | ZS:222773.1 | RAXSNOT02 | PZ-0100-P,2194,11/12/1998 |
| 1300 | hu00538491 | 2328033 | IB:124382.6 | COLNNOT11 | PD-0289-P,536,12/18/1996\|PD-0289-US,536,12/18/1997 |
| 1301 | hu00384197 | 579187 | IB:815661.1 | BRAVTXT05 | PR-0003-US,1793,11/13/1996\|PR-0004-P,1793,12/15/1995 |
| 1302 | rat00200270 | 700534404 | ZS:205952.1 | RAEONOT02 | PZ-0024-US,908,09/10/1998\|PZ-0052-P,908,12/11/1997 |
| 1303 | rat00094481 | 701344942 | ZS:205952.3 | RALITXT11 | PZ-0094-P,1394,09/11/1998 |
| 1304 | rat00210583 | 700525076 | ZS:205952.3 | RABMNOT01 | PZ-0022-US,1281,09/01/1998\|PZ-0051-P,1281,12/12/1997 |
| 1305 | rat00181406 | 701292649 | ZS:205952.3 | RABXNOT04 | PZ-0090-P,1274,08/12/1998 |
| 1306 | rat00028541 | 701654201 | ZS:205952.3 | RALITXT10 | PZ-0117-P,1421,05/07/1999 |
| 1307 | rat00216462 | 700289551 | ZS:205952.3 | RAEPNOT01 | PZ-0015-P,230,08/05/1997\|PZ-0015-US,230,08/04/1998 |
| 1308 | rat00133499 | 700415338 | ZS:205952.3 | RAHTNOT01 | PZ-0037-US,163,10/06/1998\|PZ-0048-P,163,11/05/1997 |
| 1309 | rat00254102 | 700538084 | ZS:205952.3 | RACONOT01 | PZ-0014-US,1757,08/05/1998\|PZ-0043-P,319,11/04/1997 |
| 1310 | rat00205869 | 700485031 | ZS:205952.3 | RALINON03 | PZ-0059-P,2164,01/16/1998\|PZ-0059-US,2164,01/12/1999 |
| 1311 | rat00151088 | 701650221 | ZS:205952.3 | RALITXT10 | PZ-0117-P,272,05/07/1999 |
| 1312 | rat00170519 | 701655217 | ZS:205952.3 | RALITXT10 | PZ-0117-P,1667,05/07/1999 |
| 1313 | rat00083093 | 700305954 | ZS:205952.3 | RALINOT01 | PZ-0026-P,1134,09/12/1997\|PZ-0026-US,1134,09/04/1998 |
| 1314 | rat00246380 | 701468078 | ZS:205952.3 | RALITXT32 | PZ-0108-P,852,02/11/1999 |
| 1315 | hu01278189 | 6027904 | LG:401992.6 | TESTNOT11 | |
| 1316 | hu01055764 | 6593314 | LG:401992.6 | UTREDIT14 | |
| 1317 | hu01211996 | 6307844 | LG:401992.6 | NERDTDN03 | |
| 1318 | hu01043275 | 6593514 | LG:401992.6 | UTREDIT14 | |
| 1319 | hu01015901 | 5303918 | LG:401992.6 | BRSTDIT01 | |
| 1320 | hu01344599 | 5885852 | LG:401992.7 | LIVRNON08 | |
| 1321 | hu00996714 | 6523764 | LG:401992.7 | CONFTDT02 | |
| 1322 | hu01111011 | 5993818 | LG:401992.7 | FTUBTUT02 | |
| 1323 | hu01349589 | 5882855 | LG:401992.7 | LIVRNON08 | |
| 1324 | hu01033003 | 6114074 | LG:401992.7 | SINITMT04 | |
| 1325 | hu00998594 | 3567250 | LG:401992.7 | BRONNOT02 | |
| 1326 | hu01103912 | 6114081 | LG:401992.7 | SINITMT04 | |
| 1327 | hu01261723 | 5943729 | LG:401992.7 | COLADIT05 | |
| 1328 | hu01263146 | 4211225 | LG:401992.7 | BRONDIT01 | |
| 1329 | hu01278213 | 5885814 | LG:401992.7 | LIVRNON08 | |
| 1330 | hu01152645 | 6605876 | LG:401992.7 | UTREDIT07 | |
| 1331 | hu01156704 | 5697593 | LG:401992.7 | BRSTTUT13 | |
| 1332 | hu01245940 | 4209022 | LG:401992.7 | BRONNOT02 | |
| 1333 | hu01082536 | 6364143 | LG:401992.7 | ARTANOT07 | |
| 1334 | hu01204479 | 3780874 | LG:401992.7 | BRAHDIT04 | |
| 1335 | hu01266736 | 5886691 | LG:401992.7 | LIVRNON08 | |
| 1336 | hu01165873 | 6512389 | LG:401992.7 | THYMDIT01 | |
| 1337 | hu01028859 | 4516729 | LG:401992.7 | SINJNOT03 | |
| 1338 | hu01168945 | 6522528 | LG:401992.7 | CONFTDT02 | |
| 1339 | hu01159774 | 6299317 | LG:401992.7 | UTREDIT07 | |
| 1340 | hu00979550 | 6354938 | LG:401992.7 | LUNGDIS03 | |
| 1341 | hu01130335 | 6411611 | LG:401992.7 | UTREDIT10 | |
| 1342 | hu01302830 | 6271241 | LG:401992.7 | BRAIFEN03 | |
| 1343 | hu01103129 | 4438756 | LG:401992.7 | SINTNOT22 | |
| 1344 | hu01019114 | 4438771 | LG:401992.7 | SINTNOT22 | |
| 1345 | hu01311940 | 4443584 | LG:401992.7 | SINDNOT01 | |
| 1346 | hu01009470 | 6098582 | LG:401992.7 | UTRENOT09 | |
| 1347 | hu01171822 | 6361222 | LG:401992.7 | CONFNOT09 | |
| 1348 | hu01285132 | 5883291 | LG:401992.7 | LIVRNON08 | |
| 1349 | hu01215652 | 6366607 | LG:401992.7 | ARTANOT07 | |
| 1350 | hu01293414 | 5954210 | LG:401992.7 | SKINTDT01 | |
| 1351 | hu01120975 | 4515822 | LG:401992.7 | SINJNOT03 | |
| 1352 | rat00036395 | 700488315 | ZS:222830.3 | RATENOT01 | PZ-0056-P,1431,12/11/1997\|PZ-0020-US,3208,07/31/1998 |
| 1353 | rat00028422 | 701728973 | ZS:222830.3 | RALITXT1A | PZ-0122-P,491,06/03/1999 |
| 1354 | rat00118389 | 700367922 | ZS:222830.3 | RAHYNOT01 | PZ-0033-P,265,10/08/1997\|PZ-0033-US,265,10/05/1998 |
| 1355 | rat00216886 | 700486083 | ZS:222830.3 | RATENOT01 | PZ-0020-US,2205,07/31/1998\|PZ-0056-P,428,12/11/1997 |
| 1356 | rat00099149 | 701484604 | ZS:222830.3 | RALITXT50 | PZ-0109-P,1372,02/11/1999 |
| 1357 | hu01012121 | 6480126 | LG:270051.8 | PROSTMC01 | |
| 1358 | hu01242117 | 6373901 | LG:270051.8 | FIBAUNT02 | |
| 1359 | hu01183411 | 5890253 | LG:270051.8 | LIVRNON08 | |
| 1360 | hu01031536 | 6546449 | LG:270051.8 | LNODNON02 | |
| 1361 | hu01213381 | 6424874 | LG:270051.8 | BRSTUNT01 | |
| 1362 | hu01149122 | 6176089 | LG:270051.8 | BMARUNT02 | |
| 1363 | hu01124822 | 5885588 | LG:270051.8 | LIVRNON08 | |
| 1364 | hu01266024 | 5884115 | LG:270051.8 | LIVRNON08 | |
| 1365 | hu01124184 | 6431732 | LG:270051.8 | LUNGNON07 | |
| 1366 | hu01086601 | 6319383 | LG:270051.8 | LUNGDIN02 | |
| 1367 | hu01070384 | 6006445 | LG:270051.8 | FIBRUNT02 | |
| 1368 | hu01073382 | 5714842 | LG:270051.8 | MASTTXT01 | |
| 1369 | hu01031739 | 6153505 | LG:270051.8 | ENDMUNT04 | |
| 1370 | hu01115358 | 6245935 | LG:270051.8 | TESTNOT17 | |
| 1371 | hu01153717 | 6306763 | LG:270051.8 | NERDTDN03 | |
| 1372 | hu01025981 | 5945146 | LG:270051.8 | COLADIT05 | |
| 1373 | hu01303793 | 5884982 | LG:270051.8 | LIVRNON08 | |
| 1374 | hu01227177 | 6322160 | LG:270051.8 | LUNGDIN02 | |
| 1375 | hu01239409 | 3154287 | LG:270051.8 | TLYMTXT02 | |
| 1376 | hu01178354 | 6375238 | LG:270051.8 | FIBAUNT02 | |
| 1377 | hu01269164 | 6246035 | LG:270051.8 | TESTNOT17 | |
| 1378 | hu01348841 | 6537280 | LG:270051.8 | OVARDIN02 | |
| 1379 | hu00531731 | 1319812 | IB:803140.1 | BLADNOT04 | PD-0202-US,565,07/31/1997\|PD-0170-P,570,05/29/1996 |
| 1380 | hu00485095 | 1389252 | IB:803140.1 | EOSINOT01 | PR-0009-P,214,06/04/1996\|PR-0009-US,214,06/03/1997 |
| 1381 | hu00443053 | 886511 | IB:464689.10 | PANCNOT05 | PD-0130-P,716,03/26/1996 |
| 1382 | hu00688152 | 3275752 | IB:464689.10 | PROSBPT06 | PD-0376-P,1041,06/02/1997\|PD-0376-US,1041,05/15/1998 |
| 1383 | hu00592769 | 767583 | IB:464689.10 | LUNGNOT04 | PD-0140-P,1377,04/10/1996\|PD-0121-US,2824,02/28/1997 |
| 1384 | hu00498963 | 2822820 | IB:464689.10 | ADRETUT06 | PD-0333-P,875,03/14/1997\|PD-0333-US,875,03/12/1998 |
| 1385 | hu00213468 | 2231189 | IB:464689.10 | PROSNOT16 | PD-0290-P,1186,12/19/1996\|PD-0290-US,1186,07/30/1997 |
| 1386 | hu00686498 | 2625106 | IB:464689.10 | PROSTUT12 | PD-0314-P,203,01/22/1997\|PD-0314-US,2152,09/04/1997 |
| 1387 | hu00154170 | 2823682 | IB:464689.10 | ADRETUT06 | PD-0333-P,1294,03/14/1997\|PD-0333-US,1294,03/12/1998 |
| 1388 | hu00747944 | 500 | IB:233861.8 | U937NOT01 | PD-0001-3-US,384,02/14/1994\|PD-0001-US,384,07/17/1992\|PD- |
| | | | | | 0001-1-PCT,442,07/16/1993\|PD-0001-1-US,442,11/19/1992 |
| 1389 | hu00685584 | 682041 | IB:233861.8 | UTRSNOT02 | PD-0095-P,3168,12/29/1995\|PD-0095-US,3168,12/23/1996 |
| 1390 | hu00547949 | 101255 | IB:233861.8 | ADRENOT01 | PD-0016-US,1342,10/14/1994 |
| 1391 | hu00030222 | 219448 | IB:233861.8 | STOMNOT01 | PD-0022-US,3159,02/07/1995 |
| 1392 | hu00720500 | 303290 | IB:233861.8 | TESTNOT04 | PD-0052-P,535,10/10/1995\|PD-0052-US,535,10/09/1996 |
| 1393 | hu00204075 | 207382 | IB:233861.8 | SPLNNOT02 | PD-0020-US,942,01/17/1995 |
| 1394 | hu00035425 | 580332 | IB:233861.8 | BRAVTXT05 | PR-0003-US,2129,11/13/1996\|PR-0004-P,2129,12/15/1995 |
| 1395 | hu00650413 | 23424 | IB:233861.8 | ADENINB01 | PD-0005-US,1903,02/04/1994 |
| 1396 | hu00315083 | 2636808 | IB:233861.8 | BONTNOT01 | PD-0302-P,642,01/27/1997\|PD-0302-US,642,01/23/1998 |
| 1397 | hu00221835 | 895100 | IB:233861.8 | BRSTNOT05 | PD-0133-P,767,03/20/1996\|PD-0133-US,767,03/20/1997 |
| 1398 | hu00497011 | 988478 | IB:233861.8 | LVENNOT03 | PD-0134-P,106,03/21/1996\|PD-0134-US,106,03/21/1997 |
| 1399 | hu00750217 | 927899 | IB:233861.8 | BRAINOT04 | PD-0132-P,1130,03/18/1996\|PD-0132-US,1130,03/17/1997 |
| 1400 | hu00397213 | 215284 | IB:233861.8 | STOMNOT01 | PD-0022-US,785,02/07/1995 |
| 1401 | hu00256912 | 21437 | IB:233861.8 | ADENINB01 | PD-0005-US,609,02/04/1994 |
| 1402 | hu00181475 | 24011 | IB:233861.8 | ADENINB01 | PD-0005-US,2283,02/04/1994 |
| 1403 | hu00449580 | 21857 | IB:233861.8 | ADENINB01 | PD-0005-US,858,02/04/1994 |
| 1404 | hu00716908 | 21630 | IB:233861.8 | ADENINB01 | PD-0005-US,739,02/04/1994 |
| 1405 | hu00773584 | 1818131 | IB:233861.8 | PROSNOT20 | PD-0233-P,2982,09/05/1996\|PD-0233-US,2982,09/05/1997 |
| 1406 | hu00040407 | 2663627 | IB:771292.1 | ADRENOT08 | PD-0317-P,1004,02/11/1997\|PD-0317-US,1004,02/10/1998 |
| 1407 | hu01076192 | 5920138 | LG:464689.12 | BRAIFET02 | |
| 1408 | hu00923221 | 1753287 | IB:795701.1 | LIVRTUT01 | PD-0220-P,881,07/30/1996 |
| 1409 | hu00007264 | 1812515 | IB:795701.1 | PROSTUT12 | PD-0314-US,1463,09/04/1997\|PD-0233-P,4901,09/05/1996 |
| 1410 | hu00864391 | 1710659 | IB:795701.1 | PROSNOT16 | PD-0199-P,1043,07/30/1996\|PD-0290-US,3413,07/30/1997 |
| 1411 | hu00384233 | 176197 | IB:795701.1 | TLYMNOT01 | |
| 1412 | hu00553586 | 1694075 | IB:795701.1 | COLNNOT23 | PD-0206-P,1186,08/01/1996\|PD-0206-US,1186,08/01/1997 |
| 1413 | hu00385647 | 1753396 | IB:795701.1 | LIVRTUT01 | PD-0220-P,926,07/30/1996 |
| 1414 | hu00466019 | 1753376 | IB:795701.1 | LIVRTUT01 | PD-0220-P,916,07/30/1996 |
| 1415 | hu00051632 | 2635071 | IB:401992.6 | BONTNOT01 | PD-0302-P,87,01/27/1997\|PD-0302-US,87,01/23/1998 |
| 1416 | hu00950968 | 86657 | IB:401992.8 | LIVRNOT01 | PD-0014-US,1016,09/07/1994 |
| 1417 | hu00155335 | 139171 | IB:401992.8 | LIVRNOT01 | PD-0064-P,244,10/10/1995\|PD-0064-US,244,10/09/1996 |
| 1418 | hu01034310 | 5878526 | LG:475317.2 | BRAUNOT01 | |
| 1419 | hu01304800 | 6600271 | LG:475317.2 | ARTANOT06 | |
| 1420 | hu01260071 | 5561109 | LG:475317.2 | BRSTDIT01 | |
| 1421 | hu01108602 | 5703148 | LG:475317.2 | DRGCNOT01 | |
| 1422 | hu01085123 | 4443746 | LG:475317.2 | SINDNOT01 | |
| 1423 | hu01224452 | 6428594 | LG:475317.2 | LUNGNON07 | |
| 1424 | hu01116462 | 4343561 | LG:475317.2 | SINTNOT22 | |
| 1425 | hu01332724 | 3021152 | LG:475317.2 | PROSDIN01 | |
| 1426 | hu01205581 | 6213466 | LG:475317.2 | ARTANOT06 | |
| 1427 | hu01182443 | 6376812 | LG:475317.2 | FIBAUNT01 | |
| 1428 | hu01144509 | 5700295 | LG:475317.2 | DRGCNOT01 | |
| 1429 | hu01205385 | 6364679 | LG:475317.2 | ARTANOT07 | |
| 1430 | hu01320884 | 6489032 | LG:475317.2 | MIXDUNB01 | |
| 1431 | hu01052792 | 6378287 | LG:475317.2 | FIBAUNT01 | |
| 1432 | hu01312667 | 6349775 | LG:475317.2 | LUNGDIS03 | |
| 1433 | hu01132488 | 6379980 | LG:475317.2 | FIBRUNT02 | |
| 1434 | hu00972009 | 4516928 | LG:475317.2 | SINJNOT03 | |
| 1435 | hu01094916 | 6412344 | LG:475317.2 | UTREDIT10 | |
| 1436 | hu01158348 | 4518475 | LG:475317.2 | SINJNOT03 | |
| 1437 | hu01085909 | 5704048 | LG:475317.2 | DRGCNOT01 | |
| 1438 | hu00987153 | 6363156 | LG:475317.2 | CONFNOT09 | |
| 1439 | hu00973753 | 6521229 | LG:475317.2 | CONFTDT02 | |
| 1440 | hu01328985 | 6455607 | LG:475317.2 | COLNDIC01 | |
| 1441 | hu01025091 | 6598793 | LG:475317.2 | ARTANOT06 | |
| 1442 | hu01042508 | 6208789 | LG:475317.2 | ARTANOT06 | |
| 1443 | hu01050366 | 6498438 | LG:475317.2 | COLNNOT41 | |
| 1444 | hu01316712 | 3899830 | LG:475317.2 | LUNGNON03 | |
| 1445 | hu01105484 | 4441007 | LG:475317.2 | SINTNOT22 | |
| 1446 | hu01197221 | 4668358 | LG:475317.2 | SINTNOT24 | |
| 1447 | hu01014269 | 6208589 | LG:475317.2 | ARTANOT06 | |
| 1448 | hu01119624 | 3899623 | LG:475317.2 | LUNGNON03 | |
| 1449 | hu01166370 | 4440405 | LG:475317.2 | SINTNOT22 | |
| 1450 | hu01290607 | 6491681 | LG:475317.2 | MIXDUNB01 | |
| 1451 | hu01235356 | 4443438 | LG:475317.2 | SINDNOT01 | |
| 1452 | hu01213739 | 6208777 | LG:475317.2 | ARTANOT06 | |
| 1453 | hu01012967 | 6513632 | LG:475317.2 | THYMDIT01 | |
| 1454 | hu01347308 | 6431741 | LG:475317.2 | LUNGNON07 | |
| 1455 | hu01242764 | 4438923 | LG:475317.2 | SINTNOT22 | |
| 1456 | hu01141983 | 3023596 | LG:475317.2 | PROSDIN01 | |
| 1457 | hu00981027 | 6513532 | LG:475317.2 | THYMDIT01 | |
| 1458 | hu01225836 | 6023712 | LG:475317.2 | TESTNOT11 | |
| 1459 | hu01021995 | 3022286 | LG:475317.2 | PROSDIN01 | |
| 1460 | hu01072945 | 6598639 | LG:475317.2 | ARTANOT06 | |
| 1461 | hu01335646 | 5692805 | LG:475317.2 | BRAUNOT02 | |
| 1462 | hu01252649 | 5702665 | LG:475317.2 | DRGCNOT01 | |
| 1463 | hu00971282 | 5562537 | LG:475317.2 | BRSTDIT01 | |
| 1464 | hu01294428 | 6169636 | LG:475317.2 | UTRSTDT01 | |
| 1465 | hu00991381 | 6376628 | LG:475317.2 | FIBAUNT01 | |
| 1466 | hu01304779 | 6023337 | LG:475317.2 | TESTNOT11 | |
| 1467 | hu01179067 | 6384872 | LG:475317.2 | FIBRUNT02 | |
| 1468 | hu00992273 | 6363655 | LG:475317.2 | CONFNOT09 | |
| 1469 | hu01094568 | 6010162 | LG:475317.2 | FIBRUNT02 | |
| 1470 | hu01286548 | 4517845 | LG:475317.2 | SINJNOT03 | |
| 1471 | hu01189762 | 3019790 | LG:475317.2 | PROSDIN01 | |
| 1472 | hu00378272 | 1455594 | IB:798928.1 | COLNFET02 | PD-0168-US,1623,05/21/1997\|PD-0195-P,72,06/19/1996 |
| 1473 | hu00834376 | 1433063 | IB:798928.1 | BEPINON01 | PD-0181-P,1341.05/30/1996\|PD-0181-US,1341,05/28/1997 |
| 1474 | hu01095213 | 6422373 | LG:464689.5 | BRSTUNT01 | |
| 1475 | hu01109550 | 6421660 | LG:464689.5 | BRSTUNT01 | |
| 1476 | hu00979473 | 6298033 | LG:464689.5 | UTREDIT07 | |
| 1477 | hu00988392 | 4121984 | LG:464689.5 | BRSTTUT25 | |
| 1478 | hu01006476 | 6134672 | LG:464689.5 | BMARTXT02 | |
| 1479 | hu01224429 | 6421768 | LG:464689.5 | BRSTUNT01 | |
| 1480 | hu01222954 | 6100330 | LG:464689.5 | UTRENOT09 | |
| 1481 | hu01199977 | 6559430 | LG:464689.5 | BRAFNON02 | |
| 1482 | hu01064917 | 5873539 | LG:464689.5 | COLTDIT04 | |
| 1483 | hu01253388 | 6053960 | LG:464689.5 | BRAENOT04 | |
| 1484 | hu01212406 | 6426224 | LG:464689.5 | LUNGNON07 | |
| 1485 | hu01298669 | 6310337 | LG:464689.8 | NERDTDN03 | |
| 1486 | hu00276005 | 715790 | IB:401992.10 | PROSTUT01 | PD-0138-P,636,03/25/1996\|PD-0138-US,636,03/25/1997\|PD-0139- |
| | | | | | P,636,04/10/1996 |
| 1487 | hu00469919 | 2782796 | IB:401992.10 | BRSTNOT13 | PD-0335-P,221.03/21/1997\|PD-0335-US,221,03/20/1998 |
| 1488 | hu00546016 | 2822929 | IB:401992.10 | ADRETUT06 | PD-0333-P,926,03/14/1997\|PD-0333-US,926,03/12/1998 |
| 1489 | hu00372163 | 2096038 | IB:401992.10 | BRAITUT02 | PD-0111-1-US,3758,12/05/1997\|PD-0275-P,4218,12/06/1996 |
| 1490 | hu00643640 | 2219006 | IB:401992.10 | LUNGNOT18 | PD-0263-P,229,10/28/1996\|PD-0263-US,229,10/28/1996 |
| 1491 | hu00648878 | 89474 | IB:401992.10 | LIVRNOT01 | PD-0014-US,2734,09/07/1994 |
| 1492 | hu00229760 | 714309 | IB:401992.10 | PROSTUT01 | PD-0138-P,179,03/25/1996\|PD-0138-US,179,03/25/1997\|PD-0139- |
| | | | | | P,179,04/10/1996 |
| 1493 | hu00285912 | 715581 | IB:401992.10 | PROSTUT01 | PD-0138-P,570,03/25/1996\|PD-0138-US,570,03/25/1997\|PD-0139- |
| | | | | | P,570,04/10/1996 |
| 1494 | hu00140054 | 2085627 | IB:401992.10 | PANCNOT04 | PD-0291-P,188,12/17/1996 |
| 1495 | hu00048012 | 88118 | IB:401992.10 | LIVRNOT01 | PD-0014-US,1935,09/07/1994 |
| 1496 | hu00954177 | 2815429 | IB:401992.10 | OVARNOT10 | PD-0341-P,2121,03/20/1997\|PD-0341-US,2121,03/19/1998 |
| 1497 | hu00062127 | 760074 | IB:401992.10 | BRAITUT02 | PD-0111-1-US,3177,12/05/1997\|PD-0111-P,995,02/22/1996\|PD- |
| | | | | | 0111-US,995,02/21/1997 |
| 1498 | hu00046072 | 2025847 | IB:475317.3 | KERANOT02 | PD-0116-1-US,1953,10/24/1997\|PD-0260-P,531,10/25/1996 |
| 1499 | hu00205473 | 3343173 | IB:475317.3 | SPLNNOT09 | PD-0378-P,846,06/05/1997\|PD-0378-US,846,05/19/1998 |
| 1500 | hu00569891 | 274309 | IB:475317.3 | PANCDIT03 | PD-0054-P,230,10/10/1995\|PD-0054-US,230,10/08/1996 |
| 1501 | hu00779638 | 2601745 | IB:475317.3 | UTRSNOT10 | PD-0316-P,1295,01/29/1997\|PD-0316-US,1295,01/26/1998 |
| 1502 | hu00006783 | 2892508 | IB:475317.3 | LUNGFET04 | PD-0357-P,1767,04/16/1997\|PD-0357-US,1767,04/08/1998 |
| 1503 | hu00393703 | 1362055 | IB:475317.3 | LUNGNOT12 | PD-0188-P,721,06/19/1997\|PD-0188-US,721,06/18/1997 |
| 1504 | hu00302668 | 2460609 | IB:475317.3 | THYRNOT08 | PD-0394-P,521,07/02/1997\|PD-0394-US,521,06/24/1998 |
| 1505 | hu00560726 | 2715415 | IB:475317.3 | THYRNOT09 | PD-0331-P,361,02/13/1997\|PD-0331-US,361,02/10/1998 |
| 1506 | hu00012332 | 3364921 | IB:475317.3 | PROSBPT02 | PD-0390-P,1478,06/30/1997\|PD-0390-US,1478,06/26/1998 |
| 1507 | hu00732727 | 606092 | IB:475317.3 | BRSTTUT01 | PD-0061-P,2796,11/15/1995\|PD-0061-US,2796,11/15/1996 |
| 1508 | hu00336872 | 274308 | IB:475317.3 | PANCDIT03 | PD-0054-P,229,10/10/1995\|PD-0054-US,229,10/08/1996 |
| 1509 | hu00851294 | 3358609 | IB:475317.3 | PROSTUT16 | PD-0391-P,2949,06/30/1997\|PD-0391-US,2949,06/19/1998 |
| 1510 | hu00049358 | 2640032 | IB:475317.3 | LUNGTUT08 | PD-0326-P,108,03/07/1997\|PD-0326-US,108,03/04/1997 |
| 1511 | hu00489775 | 2731063 | IB:475317.5 | OVARTUT04 | PD-0313-P,605,01/22/1997\|PD-0313-US,605,01/22/1998 |
| 1512 | hu00041104 | 2184244 | IB:475317.5 | SININOT01 | PD-0267-P,1635,10/31/1996\|PD-0267-US,1635,10/29/1997 |
| 1513 | hu00141131 | 3204456 | IB:475317.5 | PENCNOT03 | PD-0375-P,2021,06/02/1997\|PD-0375-US,2021,05/08/1998 |
| 1514 | hu00260911 | 849715 | IB:475317.5 | NGANNOT01 | PD-0123-US,2904,02/28/1997\|PD-0123-P,381,02/28/1996 |
| 1515 | hu00518080 | 2637127 | IB:475317.5 | BONTNOT01 | PD-0302-P,735,01/27/1997\|PD-0302-US,735,01/23/1998 |
| 1516 | hu00465900 | 2531872 | IB:475317.5 | GBLANOT02 | PD-0306-P,1427,01/27/1997\|PD-0306-US,1427,01/23/1998 |
| 1517 | hu00381435 | 2466966 | IB:475317.5 | THYRNOT08 | PD-0394-P,2541,07/02/1997\|PD-0394-US,2541,06/24/1998 |
| 1518 | hu00268512 | 855636 | IB:475317.5 | NGANNOT01 | PD-0123-US,1421,02/28/1997\|PD-0125-P,1421,03/07/1996 |
| 1519 | hu00639007 | 3200905 | IB:475317.5 | PENCNOT02 | PD-0375-P,693,06/02/1997\|PD-0375-US,693,05/08/1998 |
| 1520 | hu00285128 | 3045769 | IB:475317.5 | HEAANOT01 | PD-0351-P,1313,04/25/1998\|PD-0351-US,1313,04/23/1998 |
| 1521 | hu00485343 | 205471 | IB:475317.5 | MPHGNOT02 | PD-0040-P,1994,08/01/1995 |
| 1522 | hu00933750 | 2603222 | IB:475317.5 | UTRSNOT10 | PD-0316-P,1767,01/29/1997\|PD-0316-US,1767,01/26/1998 |
| 1523 | hu00915057 | 3216147 | IB:475317.5 | TESTNOT07 | PD-0380-P,783,06/04/1997\|PD-0380-US,783,05/14/1998 |
| 1524 | hu00383850 | 3345777 | IB:475317.5 | SPLNNOT09 | PD-0378-P,1767,06/05/1997\|PD-0378-US,1767,05/19/1998 |
| 1525 | hu00634951 | 2296070 | IB:475317.5 | BRSTNOT05 | PD-0292-P,693,12/19/1996\|PD-0292-US,693,12/18/1997 |
| 1526 | hu00098416 | 2210959 | IB:475317.5 | SINTFET03 | PD-0268-P,1568,10/31/1996\|PD-0268-US,1568,10/30/1997 |
| 1527 | hu00458218 | 1721127 | IB:475317.5 | BLADNOT06 | PD-0221-US,2850,07/31/1997\|PD-0221-P,872,08/22/1996 |
| 1528 | hu00199732 | 546344 | IB:475317.5 | OVARNOT02 | PD-0045-1-US,1827,11/10/1997\|PD-0045-P,1827,10/24/1995\|PD- |
| | | | | | 0045-US,1827,10/18/1996 |
| 1529 | hu00583276 | 3685024 | IB:475317.5 | HEAANOT01 | PD-0351-US,2401,04/23/1998 |
| 1530 | hu00637386 | 3044734 | IB:475317.5 | HEAANOT01 | PD-0351-P,943,04/25/1998\|PD-0351-US,943,04/23/1998 |
| 1531 | hu00398585 | 1259249 | IB:475317.5 | MENITUT03 | PD-0147-P,1699,04/17/1996\|PD-0147-US,1699,04/17/1997 |
| 1532 | hu00688557 | 2772012 | IB:475317.5 | COLANOT02 | PD-0321-P,1627,02/12/1997\|PD-0321-P,1627,02/12/1997\|PD-0321- |
| | | | | | US,1627,02/10/1998\|PD-0321-US,1627,02/10/1998 |
| 1533 | hu00131101 | 359806 | IB:475317.5 | SYNORAB01 | PD-0069-P,1190,10/04/1995\|PD-0069-US,1190,10/04/1996 |
| 1534 | hu00010027 | 850468 | IB:475317.5 | NGANNOT01 | PD-0123-US,3132,02/28/1997\|PD-0123-P,609,02/28/1996 |
| 1535 | hu00500537 | 1814906 | IB:475317.5 | PROSNOT20 | PD-0233-P,2179,09/05/1996\|PD-0233-US,2179,09/05/1997 |
| 1536 | hu00007175 | 3949616 | IB:475317.5 | DRGCNOT01 | PD-0337-US,7592,03/05/1998 |
| 1537 | hu00452405 | 2274011 | IB:475317.5 | PROSNON01 | PD-0198-US,1629,07/28/1997\|PD-0284-P,1629,12/05/1996 |
| 1538 | hu00122835 | 350870 | IB:475317.5 | LVENNOT01 | PD-0053-P,3988,10/10/1995\|PD-0053-US,3988,10/09/1996 |
| 1539 | hu00143166 | 1863706 | IB:475317.5 | PROSNOT19 | |
| 1540 | hu00891231 | 3950806 | IB:475317.5 | DRGCNOT01 | PD-0337-US,8010,03/05/1998 |
| 1541 | hu00742056 | 1511496 | IB:475317.5 | LUNGNOT14 | PD-0208-P,1279,07/24/1996\|PD-0208-US,1279,07/23/1997 |
| 1542 | hu00930375 | 3871292 | IB:475317.5 | BMARNOT03 | PD-0218-1-US,2525,03/12/1998 |
| 1543 | hu00955063 | 2350519 | IB:475317.5 | COLSUCT01 | PD-0278-P,658,12/05/1996\|PD-0278-US,658,11/21/1997 |
| 1544 | hu00547562 | 1500749 | IB:475317.5 | SINTBST01 | PD-0209-P,906,07/23/1996\|PD-0209-US,906,07/22/1997 |
| 1545 | hu00541586 | 1430632 | IB:475317.5 | SINTBST01 | PD-0209-P,2447,07/23/1996\|PD-0209-US,2447,07/22/1997 |
| 1546 | hu00225299 | 2532363 | IB:475317.5 | GBLANOT02 | PD-0306-P,1593,01/27/1997\|PD-0306-US,1593,01/23/1998 |
| 1547 | hu00870269 | 629781 | IB:475317.5 | KIDNNOT05 | PD-0063-P,747,11/22/1995\|PD-0261-1-US,747,10/29/1997 |
| 1548 | hu00295892 | 1927213 | IB:475317.5 | BRSTNOT02 | PD-0240-P,753,09/30/1996\|PD-0240-US,753,09/29/1997 |
| 1549 | hu00619659 | 1494133 | IB:475317.5 | PROSNON01 | PD-0198-US,5405,07/28/1997\|PD-0198-P,798,07/29/1996 |
| 1550 | hu00264334 | 2664524 | IB:475317.5 | ADRENOT08 | PD-0317-P,1327,02/11/1997\|PD-0317-US,1327,02/10/1998 |
| 1551 | hu00612715 | 2635976 | IB:475317.5 | BONTNOT01 | PD-0302-P,384,01/27/1997\|PD-0302-US,384,01/23/1998 |
| 1552 | hu00653360 | 825794 | IB:475317.5 | PROSNOT06 | PD-0117-P,724,02/26/1996\|PD-0117-US,724,02/26/1997 |
| 1553 | hu00275424 | 2708985 | IB:475317.5 | PONSAZT01 | PD-0342-P,2159,03/24/1997\|PD-0342-US,2159,03/17/1998 |
| 1554 | hu00643409 | 2538760 | IB:475317.5 | BONRTUT01 | PD-0303-P,450,01/27/1997 |
| 1555 | hu00594620 | 1491886 | IB:475317.5 | PROSNON01 | PD-0198-P,2094,07/29/1996\|PD-0198-US,6701,07/28/1997 |
| 1556 | hu00493542 | 575237 | IB:475317.5 | BRAVUNT01 | PR-0003-US,750,11/13/1996\|PR-0004-P,750,12/15/1995 |
| 1557 | hu00529435 | 1909488 | IB:475317.5 | CONNTUT01 | PD-0252-P,681,10/01/1996\|PD-0254-P,681,10/04/1996 |
| 1558 | hu00359417 | 1470258 | IB:475317.5 | PANCTUT02 | PD-0191-P,1191,06/21/1996\|PD-0191-US,1191,06/19/1997 |
| 1559 | hu00547491 | 980214 | IB:475317.5 | TONGTUT01 | PD-0145-P,220,04/15/1996\|PD-0145-US,220,04/15/1997 |
| 1560 | hu00659429 | 783555 | IB:475317.5 | MYOMNOT01 | PD-0113-P,1163,02/27/1996\|PD-0113-US,1163,02/27/1997 |
| 1561 | hu00891881 | 2846664 | IB:475317.5 | DRGLNOT01 | PD-0337-P,3933,03/18/1997\|PD-0337-US,3933,03/05/1998 |
| 1562 | hu00358686 | 2587840 | IB:475317.5 | BRAITUT22 | PD-0304-P,4590,01/28/1997\|PD-0304-US,4590,01/28/1998 |
| 1563 | hu00568948 | 1417356 | IB:475317.5 | BRAINOT12 | PD-0159-P,2021,06/20/1996\|PD-0159-US,2021,06/18/1997 |
| 1564 | hu00241110 | 944899 | IB:475317.5 | RATRNOT02 | PD-0135-P,1397,03/20/1996\|PD-0135-US,1397,03/19/1997 |
| 1565 | hu00143107 | 588444 | IB:475317.5 | UTRSNOT01 | PD-0084-P,587,10/26/1995\|PD-0084-US,587,10/18/1996 |
| 1566 | hu00162134 | 3043960 | IB:475317.5 | HEAANOT01 | PD-0351-P,684,04/25/1998\|PD-0351-US,684,04/23/1998 |
| 1567 | hu00473230 | 3093611 | IB:475317.5 | BRSTNOT19 | PD-0350-P,3917,04/16/1997\|PD-0350-US,3917,04/09/1998 |
| 1568 | hu00521605 | 1259091 | IB:475317.5 | MENITUT03 | PD-0147-P,1626,04/17/1996\|PD-0147-US,1626,04/17/1997 |
| 1569 | hu00249510 | 2847230 | IB:475317.5 | DRGLNOT01 | PD-0337-P,4154,03/18/1997\|PD-0337-US,4154,03/05/1998 |
| 1570 | hu00371740 | 2540514 | IB:475317.5 | BONRTUT01 | PD-0303-P,1029,01/27/1997 |
| 1571 | hu00343962 | 3441655 | IB:475317.5 | PENCNOT06 | PD-0389-P,3323,07/02/1997\|PD-0389-US,3323,06/24/1998 |
| 1572 | hu00663094 | 2968152 | IB:475317.5 | HEAONOT02 | PD-0387-P,91,07/03/1997\|PD-0387-US,91,06/30/1998 |
| 1573 | hu00945281 | 3398972 | IB:475317.5 | UTRSNOT16 | PD-0398-P,1093,07/01/1997\|PD-0398-US,1093,06/24/1998 |
| 1574 | hu00938315 | 1307709 | IB:475317.5 | COLNFET02 | PD-0168-P,105,05/21/1996\|PD-0168-US,105,05/21/1997 |
| 1575 | hu00790652 | 699870 | IB:475317.5 | SYNORAT03 | PD-0096-P,1664,12/15/1995\|PD-0096-US,1664,12/15/1995 |
| 1576 | hu00619751 | 426967 | IB:475317.5 | BLADNOT01 | PD-0048-P,988,09/29/1995 |
| 1577 | hu00925364 | 2685492 | IB:475317.5 | LUNGNOT23 | PD-0388-P,542,07/03/1997\|PD-0388-US,542,06/19/1998 |
| 1578 | hu00852465 | 3085872 | IB:475317.5 | HEAONOT03 | PD-0352-P,535,04/18/1997\|PD-0352-US,535,04/24/1998 |
| 1579 | hu00179681 | 1391873 | IB:475317.5 | THYRNOT03 | PD-0162-P,37,05/29/1996\|PD-0162-US,37,05/27/1997 |
| 1580 | hu00492188 | 2050275 | IB:475317.5 | LIVRFET02 | PD-0262-P,569,10/31/1996 |
| 1581 | hu00097395 | 1599482 | IB:475317.5 | BLADNOT03 | |
| 1582 | hu00295593 | 851914 | IB:475317.5 | NGANNOT01 | PD-0123-US,4,02/28/1997\|PD-0125-P,4,03/07/1996 |
| 1583 | hu00834968 | 2899326 | IB:475317.5 | DRGCNOT01 | PD-0337-US,4517,03/05/1998 |
| 1584 | hu00527111 | 1720956 | IB:475317.5 | BLADNOT06 | PD-0221-US,2801,07/31/1997\|PD-0221-P,823,08/22/1996 |
| 1585 | hu00047205 | 1251167 | IB:475317.5 | LUNGFET03 | PD-0146-P,2493,04/17/1996\|PD-0146-US,2493,04/16/1997 |
| 1586 | hu00223860 | 2968428 | IB:475317.5 | HEAONOT02 | PD-0387-P,202,07/03/1997\|PD-0387-US,202,06/30/1998 |
| 1587 | hu00571413 | 2800404 | IB:475317.5 | PENCNOT01 | PD-0332-P,246,03/06/1997\|PD-0332-US,246,03/06/1998 |
| 1588 | hu00951133 | 1724869 | IB:475317.5 | PROSNOT14 | PD-0201-P,452,07/29/1996\|PD-0201-US,452,07/28/1997\|PD-0210- |
| | | | | | P,452,07/24/1996 |
| 1589 | hu00047364 | 2838913 | IB:475317.5 | DRGLNOT01 | PD-0337-P,654,03/18/1997\|PD-0337-US,654,03/05/1998 |
| 1590 | hu00363337 | 829618 | IB:475317.5 | PROSTUT04 | PD-0117-P,1906,02/26/1996\|PD-0117-US,1906,02/26/1997\|PD-0197 |
| | | | | | US,330,06/23/1997 |
| 1591 | hu00235367 | 2219374 | IB:475317.5 | LUNGNOT18 | PD-0263-P,372,10/28/1996\|PD-0263-US,372,10/28/1996 |
| 1592 | hu00489105 | 2843830 | IB:475317.5 | DRGLNOT01 | PD-0337-P,2717,03/18/1997\|PD-0337-US,2717,03/05/1998 |
| 1593 | hu00424737 | 630799 | IB:475317.5 | KIDNNOT05 | PD-0063-P,1123,11/22/1995\|PD-0261-1-US,1123,10/29/1997 |
| 1594 | hu00738027 | 880514 | IB:475317.5 | THYRNOT02 | PD-0122-P,815,02/29/1996\|PD-0122-US,815,02/28/1997 |
| 1595 | hu00765072 | 1581022 | IB:475317.5 | DUODNOT01 | PD-0207-P,895,07/29/1996\|PD-0207-US,895,07/29/1997 |
| 1596 | hu00745318 | 3094485 | IB:475317.5 | BRSTNOT19 | PD-0350-P,4257,04/16/1997\|PD-0350-US,4257,04/09/1998 |
| 1597 | hu00526466 | 2749513 | IB:475317.5 | LUNGTUT11 | PD-0326-P,1635,03/07/1997\|PD-0326-US,3202,03/04/1997 |
| 1598 | hu00783137 | 3441811 | IB:475317.5 | PENCNOT06 | PD-0389-P,3367,07/02/1997\|PD-0389-US,3367,06/24/1998 |
| 1599 | hu00860493 | 1303826 | IB:475317.5 | PLACNOT02 | PD-0167-P,597,05/30/1996\|PD-0167-US,597,05/29/1997 |
| 1600 | hu00449185 | 1343609 | IB:475317.5 | PROSNOT11 | PD-0175-P,1642,05/24/1996\|PD-0175-US,1642,05/23/1997 |
| 1601 | hu00879900 | 2782204 | IB:475317.5 | BRSTNOT13 | PD-0335-P,1,03/21/1997\|PD-0335-US,1,03/20/1998 |
| 1602 | hu00674677 | 782307 | IB:475317.5 | MYOMNOT01 | PD-0113-P,897,02/27/1996\|PD-0113-US,897,02/27/1997 |
| 1603 | hu00668674 | 2294574 | IB:475317.5 | BRSTNOT05 | PD-0292-P,91,12/19/1996\|PD-0292-US,91,12/18/1997 |
| 1604 | hu00745879 | 3439540 | IB:475317.5 | PENCNOT06 | PD-0389-P,2526,07/02/1997\|PD-0389-US,2526,06/24/1998 |
| 1605 | hu00030141 | 1991854 | IB:475317.5 | CORPNOT02 | PD-0256-P,1088,10/23/1996\|PD-0185-US,4908,06/26/1997 |
| 1606 | hu00487889 | 2683006 | IB:475317.5 | SINIUCT01 | PD-0330-P,1339,02/12/1997\|PD-0330-US,1339,02/10/1998 |
| 1607 | hu00707180 | 2747201 | IB:475317.5 | LUNGTUT11 | PD-0326-US,2428,03/04/1997\|PD-0326-P,861,03/07/1997 |
| 1608 | hu00630168 | 2405011 | IB:475317.5 | BSTMNON02 | PD-0160-1-US,2283,06/30/1998 |
| 1609 | hu00081637 | 1500748 | IB:475317.5 | SINTBST01 | PD-0209-P,905,07/23/1996\|PD-0209-US,905,07/22/1997 |
| 1610 | hu00178019 | 81829 | IB:475317.5 | SYNORAB01 | PD-0012-US,2963,08/19/1994 |
| 1611 | hu00157607 | 2059058 | IB:475317.5 | OVARNOT03 | PD-0108-1-US,2054,10/01/1997\|PD-0249-P,293,10/01/1996 |
| 1612 | hu00654282 | 3365287 | IB:475317.5 | PROSBPT02 | PD-0390-P,1622,06/30/1997\|PD-0390-US,1622,06/26/1998 |
| 1613 | hu00087850 | 2841918 | IB:475317.5 | DRGLNOT01 | PD-0337-P,1934,03/18/1997\|PD-0337-US,1934,03/05/1998 |
| 1614 | hu00194690 | 2269574 | IB:475317.5 | UTRSNOT02 | PD-0288-P,1590,12/18/1996\|PD-0288-US,1590,12/16/1997 |
| 1615 | hu00841079 | 1442809 | IB:475317.5 | THYRNOT03 | PD-0162-P,3220,05/29/1996\|PD-0162-US,3220,05/27/1997 |
| 1616 | hu00237395 | 2303642 | IB:475317.5 | BRSTNOT05 | PD-0292-P,3766,12/19/1996\|PD-0292-US,3766,12/18/1997 |
| 1617 | hu00582519 | 1704346 | IB:475317.5 | DUODNOT02 | PD-0207-P,2131,07/29/1996\|PD-0207-US,2131,07/29/1997 |
| 1618 | hu00878143 | 700454 | IB:475317.5 | SYNORAT03 | PD-0096-P,1846,12/15/1995\|PD-0096-US,1846,12/15/1995 |
| 1619 | hu00857732 | 1986414 | IB:475317.5 | LUNGAST01 | PD-0120-US,4218,02/28/1997\|PD-0247-P,872,09/27/1996 |
| 1620 | hu00295854 | 2614735 | IB:475317.5 | GBLANOT01 | PD-0338-P,348,03/14/1997\|PD-0338-US,348,03/12/1998 |
| 1621 | hu00486285 | 589828 | IB:475317.5 | UTRSNOT01 | PD-0084-P,1030,10/26/1995\|PD-0084-US,1030,10/18/1996 |
| 1622 | hu00547289 | 2899169 | IB:475317.5 | DRGCNOT01 | PD-0337-US,4447,03/05/1998 |
| 1623 | hu00557569 | 850494 | IB:475317.5 | NGANNOT01 | PD-0123-US,3140,02/28/1997\|PD-0123-P,617,02/28/1996 |
| 1624 | hu00033303 | 2683822 | IB:475317.5 | LUNGNOT23 | PD-0388-P,11,07/03/1997\|PD-0388-US,11,06/19/1998 |
| 1625 | hu00131317 | 2887324 | IB:475317.5 | SINJNOT02 | PD-0343-US,3196,03/25/1998 |
| 1626 | hu00133091 | 1282535 | IB:475317.5 | COLNNOT16 | PD-0148-P,455,04/25/1996\|PD-0148-US,455,04/25/1997 |
| 1627 | hu00285103 | 1360106 | IB:475317.5 | LUNGNOT12 | P0-0188-P,201,06/19/1997\|PD-0188-US,201,06/18/1997 |
| 1628 | hu00262270 | 2785553 | IB:475317.5 | BRSTNOT13 | PD-0335-P,1325,03/21/1997\|PD-0335-US,1325,03/20/1998 |
| 1629 | hu00390009 | 1842667 | IB:475317.5 | COLNNOT07 | PD-0243-P,661,09/25/1996 |
| 1630 | hu00463062 | 435086 | IB:475317.5 | THYRNOT01 | PD-0049-P,724,10/02/1995\|PD-0049-US,724,09/30/1996 |
| 1631 | hu00065049 | 1331643 | IB:475317.5 | PANCNOT07 | PD-0172-P,1031,05/23/1996\|PD-0172-US,1031,05/22/1997 |
| 1632 | hu00657523 | 2587870 | IB:475317.5 | BRAITUT22 | PD-0304-P,4604,01/28/1997\|PD-0304-US,4604,01/28/1998 |
| 1633 | hu00641665 | 3043852 | IB:475317.5 | HEAANOT01 | PD-0351-P,645,04/25/1998\|PD-0351-US,645,04/23/1998 |
| 1634 | hu00685460 | 1928212 | IB:475317.5 | BRSTNOT02 | PD-0240-P,1100,09/30/1996\|PD-0240-US,1100,09/29/1997 |
| 1635 | hu00701858 | 2538317 | IB:475317.5 | BONRTUT01 | PD-0303-P,281,01/27/1997 |
| 1636 | hu00492296 | 1492720 | IB:475317.5 | PROSNON01 | PD-0198-P,190,07/29/1996\|PD-0198-US,4797,07/28/1997 |
| 1637 | hu00914947 | 1361335 | IB:475317.5 | LUNGNOT12 | PD-0188-P,531,06/19/1997\|PD-0188-US,531,06/18/1997 |
| 1638 | hu00580985 | 2547050 | IB:475317.5 | UTRSNOT11 | PD-0316-P,3488,01/29/1997\|PD-0316-US,3488,01/29/1998 |
| 1639 | hu00869328 | 3043641 | IB:475317.5 | HEAANOT01 | PD-0351-P,563,04/25/1998\|PD-0351-US,563,04/23/1998 |
| 1640 | hu00306113 | 1259955 | IB:475317.5 | MENITUT03 | PD-0147-P,2013,04/17/1996\|PD-0147-US,2013,04/17/1997 |
| 1641 | hu00041492 | 946266 | IB:475317.5 | RATRNOT02 | PD-0135-P,1748,03/20/1996\|PD-0135-US,1748,03/19/1997 |
| 1642 | hu00906055 | 3012448 | IB:475317.5 | MUSCNOT07 | PD-0359-P,1065,04/11/1997\|PD-0359-US,1065,03/31/1998 |
| 1643 | hu00031696 | 420025 | IB:475317.5 | BRSTNOT01 | PD-0051-P,3000,10/02/1995 |
| 1644 | hu00801890 | 982741 | IB:475317.5 | TONGTUT01 | PD-0145-P,890,04/15/1996\|PD-0145-US,890,04/15/1997 |
| 1645 | hu00171728 | 2509023 | IB:475317.5 | CONUTUT01 | PD-0300-P,2749,12/20/1996\|PD-0300-US,2749,12/18/1997 |
| 1646 | hu00166931 | 2670392 | IB:475317.5 | ESOGTUT02 | PD-0323-P,1420,03/05/1997\|PD-0323-US,1420,03/02/1998 |
| 1647 | hu00745263 | 2735656 | IB:475317.5 | OVARNOT09 | PD-0311-P,373,01/27/1997\|PD-0311-US,373,01/23/1998 |
| 1648 | hu00551367 | 2885490 | IB:475317.5 | SINJNOT02 | PD-0343-US,2528,03/25/1998 |
| 1649 | hu00867070 | 1303890 | IB:475317.5 | PLACNOT02 | PD-0167-P,652,05/30/1996\|PD-0167-US,652,05/29/1997 |
| 1650 | hu00682084 | 872731 | IB:475317.5 | LUNGAST01 | PD-0120-US,253,02/28/1997\|PD-0124-P,253,03/13/1996 |
| 1651 | hu00363449 | 696718 | IB:475317.5 | SYNORAT03 | PD-0096-P,682,12/15/1995\|PD-0096-US,682,12/15/1995 |
| 1652 | hu00964039 | 3688517 | IB:475317.5 | HEAANOT01 | PD-0351-US,3569,04/23/1998 |
| 1653 | rat00069019 | 701093781 | ZS:214234.3 | RALUNOT02 | PZ-0103-P,935,12/10/1998 |
| 1654 | rat00253574 | 701490564 | ZS:214234.3 | RALITXT54 | PZ-0135-P,495,08/05/1999 |
| 1655 | rat00054846 | 701599183 | ZS:214234.3 | RALITXT13 | PZ-0118-P,156,05/12/1999 |
| 1656 | rat00007795 | 701616219 | ZS:214234.3 | RALITXT61 | PZ-0127-P,131,07/07/1999 |
| 1657 | rat00176617 | 700180910 | ZS:214234.3 | RAANOT01 | PZ-0011-P,4,08/06/1997\|PZ-0011-US,4,07/30/1998 |
| 1658 | rat00114994 | 701230439 | ZS:214234.3 | RASJNON03 | PZ-0098-P,662,11/12/1998 |
| 1659 | rat00209478 | 701565987 | ZS:214234.3 | RALITXT77 | PZ-0128-P,520,07/07/1999 |
| 1660 | rat00139486 | 701469043 | ZS:214234.3 | RALITXT32 | PZ-0108-P,1031,02/11/1999 |
| 1661 | rat00078966 | 700813572 | ZS:214234.3 | RAPINOT03 | PZ-0073-P,1525,03/11/1998\|PZ-0073-US,1525,03/09/1999 |
| 1662 | rat00120521 | 701624629 | ZS:214234.3 | RALITXT83 | PZ-0133-P,3150,08/10/1999 |
| 1663 | rat00213144 | 701320686 | ZS:214234.3 | RALITXT05 | PZ-0092-P,681,08/13/1998 |
| 1664 | rat00152143 | 701506292 | ZS:214234.3 | RALITXT01 | PZ-0112-P,540,03/08/1999 |
| 1665 | rat00203903 | 700361285 | ZS:214234.3 | RALMNON04 | PZ-0017-US,53,08/05/1998\|PZ-0035-P,53,10/10/1997 |
| 1666 | rat00025460 | 700182881 | ZS:214234.3 | RAADNOT01 | PZ-0011-P,736,08/06/1997\|PZ-0011-US,736,07/30/1998 |
| 1667 | rat00082034 | 700629418 | ZS:214234.3 | RAFBNOT01 | PZ-0064-P,1554,02/12/1998\|PZ-0064-US,1554,02/10/1999 |
| 1668 | rat00052515 | 701468416 | ZS:214234.3 | RALITXT32 | PZ-0108-P,908,02/11/1999 |
| 1669 | rat00117711 | 701513663 | ZS:214234.3 | RALITXT02 | PZ-0112-P,2195,03/08/1999 |
| 1670 | rat00248310 | 701605944 | ZS:214234.3 | RALITXT09 | PZ-0116-P,339,04/29/1999 |
| 1671 | rat00123355 | 701491741 | ZS:214234.3 | RALITXT54 | PZ-0135-P,746,08/05/1999 |
| 1672 | rat00005860 | 700539949 | ZS:214234.3 | RACONOT01 | PZ-0014-US,2364,08/05/1998\|PZ-0043-P,926,11/04/1997 |
| 1673 | rat00217740 | 701230808 | ZS:214234.3 | RASJNON03 | PZ-0098-P,860,11/12/1998 |
| 1674 | rat00021189 | 700272961 | ZS:214234.3 | RASJNOT01 | PZ-0039-P,782,10/08/1997\|PZ-0039-US,782,10/05/1998 |
| 1675 | rat00111777 | 701300213 | ZS:214234.3 | RABXNOT05 | PZ-0090-P,5178,08/12/1998 |
| 1676 | rat00157434 | 700628557 | ZS:214234.3 | RAFBNOT01 | PZ-0064-P,1275,02/12/1998\|PZ-0064-US,1275,02/10/1999 |
| 1677 | rat00154482 | 701318893 | ZS:214234.3 | RALITXT05 | PZ-0092-P,192,08/13/1998 |
| 1678 | rat00138760 | 700136650 | ZS:214234.3 | RALMNOT01 | PZ-0017-P,2027,08/06/1997\|PZ-0017-US,3641,08/05/1998 |
| 1679 | rat00188046 | 701605366 | ZS:214234.3 | RALITXT09 | PZ-0116-P,205,04/29/1999 |
| 1680 | rat00255844 | 701344948 | ZS:214234.3 | RALITXT11 | PZ-0094-P,1397,09/11/1998 |
| 1681 | rat00236299 | 701617705 | ZS:214234.3 | RALITXT61 | PZ-0127-P,417,07/07/1999 |
| 1682 | rat00143999 | 700303285 | ZS:214234.3 | RALINOT01 | PZ-0026-P,406,09/12/1997\|PZ-0026-US,406,09/04/1998 |
| 1683 | rat00238796 | 701445318 | ZS:214234.3 | RALITXT50 | PZ-0109-P,1983,02/11/1999 |
| 1684 | rat00040500 | 701514224 | ZS:214234.3 | RALITXT02 | PZ-0112-P,2321,03/08/1999 |
| 1685 | rat00001036 | 700148731 | ZS:214234.3 | RANPNOT01 | PZ-0008-P,1094,07/10/1997\|PZ-0008-US,1094,07/10/1998\|PZ-0009- |
| | | | | | P,1094,07/09/1997 |
| 1686 | rat00227113 | 701465349 | ZS:214234.3 | RALITXT32 | PZ-0108-P,311,02/11/1999 |
| 1687 | rat00157121 | 701517634 | ZS:214234.3 | RALITXT62 | PZ-0136-P,99,08/06/1999 |
| 1688 | rat00200366 | 701425745 | ZS:214234.3 | RALINOT02 | PZ-0114-P,7,04/08/1999 |
| 1689 | rat00236142 | 700504333 | ZS:214234.3 | RALINON07 | PZ-0060-P,1557,01/16/1998\|PZ-0059-US,3755,01/12/1999 |
| 1690 | rat00183042 | 701897857 | ZS:214234.3 | RABYUNS09 | PZ-0131-P,1039,06/29/1999 |
| 1691 | rat00016722 | 701469758 | ZS:214234.3 | RALITXT32 | PZ-0108-P,1187,02/11/1999 |
| 1692 | rat00061390 | 701441537 | ZS:214234.3 | RALINON08 | PZ-0113-P,2948,04/07/1999 |
| 1693 | rat00193403 | 700628292 | ZS:214234.3 | RAFBNOT01 | PZ-0064-P,1189,02/12/1998\|PZ-0064-US,1189,02/10/1999 |
| 1694 | rat00109704 | 701582451 | ZS:214234.3 | RALIUNT03 | PZ-0121-P,875,05/03/1999 |
| 1695 | rat00176071 | 701193139 | ZS:214234.3 | RALINOT02 | PZ-0082-P,129,05/01/1998\|PZ-0026-US,1459,09/04/1998 |
| 1696 | rat00141862 | 701417436 | ZS:214234.3 | RASLNOT01 | PZ-0107-P,45,01/14/1999 |
| 1697 | rat00155543 | 701193977 | ZS:214234.3 | RALINOT02 | PZ-0026-US,1663,09/04/1998\|PZ-0082-P,333,05/01/1998 |
| 1698 | rat00036447 | 700609902 | ZS:214234.3 | RALINOH01 | PZ-0053-P,832,12/12/1997\|PZ-0053-US,832,12/11/1998 |
| 1699 | rat00248561 | 700361875 | ZS:214234.3 | RALMNON04 | PZ-0017-US,269,08/05/1998\|PZ-0035-P,269,10/10/1997 |
| 1700 | rat00095634 | 701246479 | ZS:214234.3 | RALINOH02 | PZ-0091-P,2402,10/27/1998 |
| 1701 | rat00233793 | 701709889 | ZS:214234.3 | RALITXT92 | PZ-0136-P,1175,08/06/1999 |
| 1702 | rat00251696 | 700503636 | ZS:214234.3 | RALINON07 | PZ-0060-P,1267,01/16/1998\|PZ-0059-US,3465,01/12/1999 |
| 1703 | rat00098747 | 701467565 | ZS:214234.3 | RALITXT32 | PZ-0108-P,753,02/11/1999 |
| 1704 | rat00174779 | 700913464 | ZS:214234.3 | RAXSNOT02 | PZ-0100-P,3244,11/12/1998 |
| 1705 | rat00032013 | 701511270 | ZS:214234.3 | RALITXT02 | PZ-0112-P,1699,03/08/1999 |
| 1706 | rat00130024 | 701510462 | ZS:214234.3 | RALITXT02 | PZ-0112-P,1544,03/08/1999 |
| 1707 | rat00199788 | 701485744 | ZS:214234.3 | RALITXT50 | PZ-0109-P,1614,02/11/1999 |
| 1708 | rat00040710 | 701510883 | ZS:214234.3 | RALITXT02 | PZ-0112-P,1626,03/08/1999 |
| 1709 | rat00237200 | 700629420 | ZS:214234.3 | RAFBNOT01 | PZ-0064-P,1555,02/12/1998\|PZ-0064-US,1555,02/10/1999 |
| 1710 | rat00102606 | 701474055 | ZS:214234.3 | RALITXT41 | PZ-0123-P,798,06/04/1999 |
| 1711 | rat00116043 | 701465268 | ZS:214234.3 | RALITXT32 | PZ-0108-P,293,02/11/1999 |
| 1712 | rat00193013 | 700297261 | ZS:214234.3 | RAEGNOT01 | PZ-0023-P,632,09/11/1997\|PZ-0023-US,632,09/01/1998 |
| 1713 | rat00193411 | 700624973 | ZS:214234.3 | RAFBNOT01 | PZ-0064-P,169,02/12/1998\|PZ-0064-US,169,02/10/1999 |
| 1714 | rat00058115 | 701466020 | ZS:214234.3 | RALITXT32 | PZ-0108-P,418,02/11/1999 |
| 1715 | rat00158342 | 700931834 | ZS:214234.3 | RALINON03 | PZ-0075-US,1525,03/09/1999\|PZ-0075-P,585,03/10/1998 |
| 1716 | rat00186206 | 701466523 | ZS:214234.3 | RALITXT32 | PZ-0108-P,526,02/11/1999 |
| 1717 | rat00062852 | 701470845 | ZS:214234.3 | RALITXT41 | PZ-0123-P,85,06/04/1999 |
| 1718 | rat00175712 | 701490988 | ZS:214234.3 | RALITXT54 | PZ-0135-P,587,08/05/1999 |
| 1719 | rat00005882 | 700769675 | ZS:214234.3 | RAHYNOT02 | PZ-0078-P,1942,04/06/1998\|PZ-0033-US,3606,10/05/1998 |
| 1720 | rat00223962 | 701435210 | ZS:214234.3 | RALITXT06 | PZ-0115-P,1304,04/08/1999 |
| 1721 | rat00224772 | 701487591 | ZS:214234.3 | RALITXT50 | PZ-0109-P,1889,02/11/1999 |
| 1722 | rat00051487 | 700821620 | ZS:214234.3 | RAESNOT02 | PZ-0032-US,2179,10/07/1998\|PZ-0074-P,942,03/13/1998 |
| 1723 | rat00154780 | 701427613 | ZS:214234.3 | RALINOT02 | PZ-0114-P,373,04/08/1999 |
| 1724 | rat00154826 | 701560653 | ZS:214234.3 | RALITXT76 | PZ-0127-P,1522,07/07/1999 |
| 1725 | rat00059481 | 701512645 | ZS:214234.3 | RALITXT02 | PZ-0112-P,1986,03/08/1999 |
| 1726 | rat00223849 | 700769805 | ZS:214234.3 | RAHYNOT02 | PZ-0078-P,2006,04/06/1998\|PZ-0033-US,3670,10/05/1998 |
| 1727 | rat00157296 | 701514225 | ZS:214234.3 | RALITXT02 | PZ-0112-P,2322,03/08/1999 |
| 1728 | rat00057549 | 700290655 | ZS:214234.3 | RAEPNOT01 | PZ-0015-P,676,08/05/1997\|PZ-0015-US,676,08/04/1998 |
| 1729 | rat00167098 | 700628888 | ZS:214234.3 | RAFBNOT01 | PZ-0064-P,1387,02/12/1998\|PZ-0064-US,1387,02/10/1999 |
| 1730 | rat00147858 | 701615842 | ZS:214234.3 | RALITXT61 | PZ-0127-P,31,07/07/1999 |
| 1731 | rat00063988 | 700821482 | ZS:214234.3 | RAESNOT02 | PZ-0032-US,2140,10/07/1998\|PZ-0074-P,903,03/13/1998 |
| 1732 | rat00000043 | 700143947 | ZS:214234.3 | RAPRNOT01 | PZ-0009-US,392,07/09/1998 |
| 1733 | rat00130034 | 701492871 | ZS:214234.3 | RALITXT54 | PZ-0135-P,977,08/05/1999 |
| 1734 | rat00123222 | 701466168 | ZS:214234.3 | RALITXT32 | PZ-0108-P,459,02/11/1999 |
| 1735 | rat00002308 | 701243343 | ZS:214234.3 | RALINOH02 | PZ-0091-P,1200,10/27/1998 |
| 1736 | rat00205115 | 701491093 | ZS:214234.3 | RALITXT54 | PZ-0135-P,604,08/05/1999 |
| 1737 | rat00130832 | 701560741 | ZS:214234.3 | RALITXT76 | PZ-0127-P,1539,07/07/1999 |
| 1738 | rat00191402 | 700640940 | ZS:214234.3 | RABUNOT01 | PZ-0057-US,2695,01/11/1999\|PZ-0058-P,337,01/16/1998 |
| 1739 | rat00044081 | 701649230 | ZS:214234.3 | RALITXT40 | PZ-0120-P,1474,05/06/1999 |
| 1740 | rat00065250 | 701940939 | ZS:214234.3 | RALITXT1K | PZ-0134-P,173,08/12/1999 |
| 1741 | rat00016704 | 701393081 | ZS:214234.3 | RALITXT14 | PZ-0094-P,2356,09/11/1998 |
| 1742 | rat00100918 | 701397339 | ZS:214234.3 | RALITXT14 | PZ-0094-P,3119,09/11/1998 |
| 1743 | rat00205552 | 700502073 | ZS:214234.3 | RALINON07 | PZ-0059-US,2778,01/12/1999\|PZ-0060-P,580,01/16/1998 |
| 1744 | rat00108751 | 700934938 | ZS:214234.3 | RALINON03 | PZ-0075-P,1974,03/10/1998\|PZ-0075-US,2914,03/09/1999 |
| 1745 | rat00250825 | 701714541 | ZS:214234.3 | RABYUNN01 | PZ-0124-P,1224,06/29/1999 |
| 1746 | rat00124790 | 701343424 | ZS:214234.3 | RALITXT11 | PZ-0094-P,934,09/11/1998 |
| 1747 | rat00140918 | 700229933 | ZS:214234.3 | RACONOT01 | PZ-0014-P,913,08/08/1997\|PZ-0014-US,913,08/05/1998 |
| 1748 | rat00049451 | 701093696 | ZS:214234.3 | RALUNOT02 | PZ-0103-P,903,12/10/1998 |
| 1749 | rat00139362 | 701610104 | ZS:214234.3 | RALITXT09 | PZ-0116-P,1486,04/29/1999 |
| 1750 | rat00172338 | 700288991 | ZS:214234.3 | RASDNOT01 | PZ-0027-P,1162,09/08/1997\|PZ-0027-US,1162,09/08/1998 |
| 1751 | rat00207294 | 700136250 | ZS:214234.3 | RALMNOT01 | PZ-0017-P,1868,08/06/1997\|PZ-0017-US,3482,08/05/1998 |
| 1752 | hu00859025 | 1371843 | IB:464689.8 | BSTMNON02 | PD-0160-1-US,814,06/30/1998\|PD-0160-P,814,04/26/1996 |
| 1753 | hu00151711 | 2688148 | IB:464689.9 | LUNGNOT23 | PD-0388-P,1522,07/03/1997\|PD-0388-US,1522,06/19/1998 |
| 1754 | hu00268925 | 2875106 | IB:464689.9 | THYRNOT10 | PD-0395-P,2766,07/02/1997\|PD-0395-US,2766,06/23/1998 |
| 1755 | hu00873888 | 587415 | IB:464689.9 | UTRSNOT01 | PD-0084-P,277,10/26/1995\|PD-0084-US,277,10/18/1996 |
| 1756 | rat00196343 | 700190695 | ZS:215574.1 | RATENOT01 | PZ-0020-P,1436,08/06/1997\|PZ-0020-US,1436,07/31/1998 |
| 1757 | rat00162259 | 701283151 | ZS:215574.2 | RABXNOT02 | PZ-0087-P,1737,08/11/1998 |
| 1758 | rat00149803 | 700228775 | ZS:215574.2 | RACONOT01 | PZ-0014-P,405,08/08/1997\|PZ-0014-US,405,08/05/1998 |
| 1759 | rat00047971 | 700417976 | ZS:215574.2 | RAHTNOT01 | PZ-0037-US,1072,10/06/1998\|PZ-0048-P,1072,11/05/1997 |
| 1760 | rat00057844 | 700179881 | ZS:215574.2 | RALUNOT01 | PZ-0006-P,720,07/10/1997\|PZ-0006-US,720,07/10/1998 |
| 1761 | rat00096841 | 701023034 | ZS:215574.2 | RAFANOT02 | PZ-0101-P,432,12/10/1998 |
| 1762 | rat00058693 | 700524975 | ZS:215574.2 | RABMNOT01 | PZ-0022-US,1257,09/01/1998\|PZ-0051-P,1257,12/12/1997 |
| 1763 | rat00161304 | 700268791 | ZS:215574.2 | RAADNOT03 | PZ-0011-US,1485,07/30/1998\|PZ-0031-P,292,10/10/1997 |
| 1764 | rat00188358 | 700066924 | ZS:215574.2 | RABTNOT01 | PZ-0001-P,1054,06/12/1997\|PZ-0001-US,1054,06/08/1998 |
| 1765 | rat00204174 | 700294785 | ZS:215574.2 | RABFNOT02 | PZ-0013-P,927,08/08/1997\|PZ-0013-US,927,08/07/1998 |
| 1766 | rat00235404 | 700478479 | ZS:215574.2 | RATHNOT02 | PZ-0049-US,1616,11/06/1998\|PZ-0072-P,75,03/12/1998 |
| 1767 | rat00144852 | 700059848 | ZS:215574.2 | RAKINOT02 | PZ-0002-US,1171,06/09/1998\|PZ-0002-P,429,06/13/1997 |
| 1768 | rat00062953 | 700627741 | ZS:215574.3 | RAFBNOT01 | PZ-0064-P,1026,02/12/1998\|PZ-0064-US,1026,02/10/1999 |
| 1769 | rat00192832 | 700627895 | ZS:215574.3 | RAFBNOT01 | PZ-0064-P,1068,02/12/1998\|PZ-0064-US,1068,02/10/1999 |
| 1770 | rat00039681 | 701433720 | ZS:215574.3 | RALITXT06 | PZ-0115-P,1020,04/08/1999 |
| 1771 | rat00115488 | 700498943 | ZS:215574.3 | RAHINOT01 | PZ-0025-US,1756,09/09/1998\|PZ-0044-P,1756,11/04/1997 |
| 1772 | rat00193491 | 700280214 | ZS:215574.3 | RAHYNOT01 | PZ-0033-P,9,10/08/1997\|PZ-0033-US,9,10/05/1998 |
| 1773 | rat00167807 | 700501772 | ZS:215574.3 | RALINON07 | PZ-0059-US,2636,01/12/1999\|PZ-0060-P,438,01/16/1998 |
| 1774 | rat00086790 | 700177732 | ZS:215574.3 | RASINOT01 | PZ-0005-P,1156,07/09/1997\|PZ-0005-US,1156,07/09/1998 |
| 1775 | rat00199525 | 701881119 | ZS:215574.3 | RALITXS02 | PZ-0133-P,1421,08/10/1999 |
| 1776 | rat00257665 | 700533367 | ZS:215574.3 | RAEONOT02 | PZ-0024-US,491,09/10/1998\|PZ-0052-P,491,12/11/1997 |
| 1777 | rat00158777 | 701294328 | ZS:215574.3 | RABXNOT04 | PZ-0090-P,2166,08/12/1998 |
| 1778 | rat00102523 | 700364549 | ZS:215574.3 | RAMGNOT01 | PZ-0046-P,7,11/04/1997\|PZ-0046-US,7,11/03/1998 |
| 1779 | rat00202063 | 700307292 | ZS:215574.3 | RASDNOT02 | PZ-0027-US,1497,09/08/1998\|PZ-0028-P,243,09/08/1997 |
| 1780 | rat00067043 | 701475321 | ZS:215574.3 | RALITXT41 | PZ-0123-P,1091,06/04/1999 |
| 1781 | rat00252273 | 701944916 | ZS:215574.3 | RALITXT1K | PZ-0134-P,951,08/12/1999 |
| 1782 | rat00060259 | 701285043 | ZS:215574.3 | RABXNOT03 | PZ-0087-P,2643,08/11/1998 |
| 1783 | rat00027860 | 701434268 | ZS:215574.3 | RALITXT06 | PZ-0115-P,1155,04/08/1999 |
| 1784 | rat00055734 | 700628168 | ZS:215574.3 | RAFBNOT01 | PZ-0064-P,1151,02/12/1998\|PZ-0064-US,1151,02/10/1999 |
| 1785 | rat00258360 | 701192218 | ZS:215574.3 | RACONON04 | PZ-0085-P,3542,06/11/1998 |
| 1786 | rat00166056 | 700534239 | ZS:215574.3 | RAEONOT02 | PZ-0024-US,836,09/10/1998\|PZ-0052-P,836,12/11/1997 |
| 1787 | rat00152547 | 700326287 | ZS:215574.3 | RASNNOT01 | PZ-0054-US,2766,12/10/1998\|PZ-0095-P,384,09/09/1998 |
| 1788 | hu01041273 | 4448044 | LG:233861.1 | SINDNOT01 | |
| 1789 | hu01084118 | 6538054 | LG:233861.5 | OVARDIN02 | |
| 1790 | hu01216492 | 5660005 | LG:233861.5 | BSCNNOT03 | |
| 1791 | hu01229687 | 6510029 | LG:233861.7 | BRAHNOT02 | |
| 1792 | hu01067795 | 6480568 | LG:233861.7 | PROSTMC01 | |
| 1793 | hu00967356 | 5812622 | LG:233861.7 | PROSTUS23 | |
| 1794 | hu00989750 | 6354669 | LG:233861.7 | LUNGDIS03 | |
| 1795 | hu01310232 | 4443286 | LG:233861.7 | SINDNOT01 | |
| 1796 | hu01041413 | 6060942 | LG:233861.7 | BRAENOT04 | |
| 1797 | hu01280112 | 6093338 | LG:233861.7 | THP1TXT03 | |
| 1798 | hu01312017 | 5943867 | LG:233861.7 | COLADIT05 | |
| 1799 | hu01303116 | 6312930 | LG:233861.7 | NERDTDN03 | |
| 1800 | hu01214233 | 6127343 | LG:233861.7 | BRAHNON05 | |
| 1801 | hu01217015 | 6131166 | LG:233861.7 | BMARTXT02 | |
| 1802 | hu01049111 | 6059741 | LG:233861.7 | BRAENOT04 | |
| 1803 | hu01107530 | 6012595 | LG:233861.7 | FIBRUNT02 | |
| 1804 | hu01136440 | 5946232 | LG:233861.7 | LIVRTUN04 | |
| 1805 | hu01220068 | 6350367 | LG:233861.7 | LUNGDIS03 | |
| 1806 | hu01141615 | 6013556 | LG:233861.7 | FIBRUNT02 | |
| 1807 | hu01062073 | 6383511 | LG:233861.7 | FIBRUNT02 | |
| 1808 | hu01100792 | 6121245 | LG:233861.7 | BRAHNON05 | |
| 1809 | hu01001783 | 6566762 | LG:233861.7 | MCLDTXN05 | |
| 1810 | hu01238043 | 6350903 | LG:233861.7 | LUNGDIS03 | |
| 1811 | hu01090714 | 6192825 | LG:233861.7 | MUSCDIN06 | |
| 1812 | hu01304754 | 6383743 | LG:233861.7 | FIBRUNT02 | |
| 1813 | hu01214188 | 5952122 | LG:233861.7 | LIVRTUN04 | |
| 1814 | hu01085182 | 5992338 | LG:233861.7 | FTUBTUT02 | |
| 1815 | hu01104677 | 5876091 | LG:233861.7 | BRAUNOT01 | |
| 1816 | hu01065329 | 5887762 | LG:233861.7 | LIVRNON08 | |
| 1817 | hu01315259 | 6264693 | LG:233861.7 | MCLDTXN03 | |
| 1818 | hu01184635 | 1894667 | LG:233861.7 | THP1TXT04 | |
| 1819 | hu01261756 | 6298742 | LG:233861.7 | UTREDIT07 | |
| 1820 | hu01318645 | 5887801 | LG:233861.7 | LIVRNON08 | |
| 1821 | hu01284328 | 6370049 | LG:233861.7 | ENDINOT01 | |
| 1822 | hu01175852 | 6498016 | LG:233861.7 | COLNNOT41 | |
| 1823 | hu01265078 | 6213424 | LG:233861.7 | ARTANOT06 | |
| 1824 | hu01189523 | 5844137 | LG:233861.7 | BRAENOT04 | |
| 1825 | hu01291278 | 6424826 | LG:233861.7 | BRSTUNT01 | |
| 1826 | hu01250699 | 6058776 | LG:233861.7 | BRAENOT04 | |
| 1827 | hu01281848 | 5839201 | LG:233861.7 | MCLDTXT02 | |
| 1828 | hu00971781 | 6510129 | LG:233861.7 | BRAHNOT02 | |
| 1829 | hu00271788 | 3105267 | IB:794649.2 | COLAUCT01 | PD-0371-P,1639,05/15/1997\|PD-0371-US,1639,05/11/1998 |
| 1830 | rat00189898 | 700062910 | ZS:219574.1 | RAKINOT02 | PZ-0002-P,1709,06/13/1997\|PZ-0002-US,2451,06/09/1998 |
| 1831 | rat00167530 | 700067568 | ZS:219574.1 | RABTNOT01 | PZ-0001-P,1287,06/12/1997\|PZ-0001-US,1287,06/08/1998 |
| 1832 | rat00018842 | 700308813 | ZS:219574.1 | RASDNOT02 | PZ-0027-US,1947,09/08/1998\|PZ-0028-P,693,09/08/1997 |
| 1833 | rat00199561 | 700180966 | ZS:219574.1 | RAADNOT01 | PZ-0011-P,28,08/06/1997\|PZ-0011-US,28,07/30/1998 |
| 1834 | rat00164141 | 700510020 | ZS:219574.2 | RASLTXT01 | PZ-0061-P,1456,01/16/1998\|PZ-0061-US,3398,01/14/1999 |
| 1835 | rat00012080 | 701335324 | ZS:219574.2 | RALINON08 | PZ-0034-US,2532,10/08/1998 |
| 1836 | rat00061336 | 700918201 | ZS:219574.2 | RAXSNOT01 | PZ-0100-P,1114,11/12/1998 |
| 1837 | rat00171128 | 701286045 | ZS:219574.2 | RABXNOT03 | PZ-0087-P,3113,08/11/1998 |
| 1838 | rat00104497 | 700290714 | ZS:208685.1 | RAEPNOT01 | PZ-0015-P,701,08/05/1997\|PZ-0015-US,701,08/04/1998 |
| 1839 | rat00241074 | 700190967 | ZS:208685.1 | RATENOT01 | PZ-0020-P,1584,08/06/1997\|PZ-0020-US,1584,07/31/1998 |
| 1840 | rat00052948 | 700302994 | ZS:208685.1 | RALINOT01 | PZ-0026-P,324,09/12/1997\|PZ-0026-US,324,09/04/1998 |
| 1841 | hu00408896 | 2010193 | IB:413486.3 | TESTNOT03 | PD-0270-P,996,10/31/1996\|PD-0270-US,996,10/29/1997 |
| 1842 | hu00686297 | 2823003 | IB:413486.3 | ADRETUT06 | PD-0333-P,972,03/14/1997\|PD-0333-US,972,03/12/1998 |
| 1843 | hu00888266 | 59307 | IB:413486.3 | | PD-0009-US,943,07/08/1994 |
| 1844 | hu00612156 | 2820251 | IB:413486.3 | BRSTNOT14 | PD-0335-P,3895,03/21/1997\|PD-0335-US,3895,03/20/1998 |
| 1845 | hu00404328 | 1240147 | IB:413486.3 | LUNGTUT02 | PD-0158-P,1778,04/22/1996 |
| 1846 | hu00052455 | 82699 | IB:413486.3 | HUVESTB01 | PD-0003-3-US,6156,08/26/1994 |
| 1847 | hu00124465 | 2825725 | IB:413486.3 | ADRETUT06 | PD-0333-P,2268,03/14/1997\|PD-0333-US,2268,03/12/1998 |
| 1848 | hu00494520 | 442 | IB:413486.3 | U937NOT01 | PD-0001-3-US,327,02/14/1994\|PD-0001-US,327,07/17/1992\|PD- |
| | | | | | 0001-1-PCT,385,07/16/1993\|PD-0001-1-US,385,11/19/1992 |
| 1849 | hu00808027 | 725067 | IB:413486.3 | SYNOOAT01 | PD-0099-P,830,12/19/1995\|PD-0099-US,830,12/19/1996 |
| 1850 | hu00025766 | 82700 | IB:413486.3 | HUVESTB01 | PD-0003-3-US,6157,08/26/1994 |
| 1851 | hu00453158 | 364676 | IB:413486.3 | PROSNOT01 | PD-0046-P,1620,09/29/1995\|PD-0046-US,1620,09/27/1996 |
| 1852 | hu00316197 | 1686 | IB:413486.3 | U937NOT01 | PD-0001-1-PCT,1532,07/16/1993\|PD-0001-1- |
| | | | | | US,1532,11/19/1992\|PD-0001-3-US,1532,02/14/1994 |
| 1853 | hu00087490 | 78129 | IB:413486.3 | SYNORAB01 | PD-0012-US,757,08/19/1994 |
| 1854 | hu00475372 | 2971626 | IB:413486.3 | HEAONOT02 | PD-0387-P,1464,07/03/1997\|PD-0387-US,1464,06/30/1998 |
| 1855 | hu00616168 | 189009 | IB:413486.3 | CARDNOT01 | PD-0019-US,3142,01/05/1995 |
| 1856 | hu00080866 | 149464 | IB:413486.3 | FIBRNGT02 | PD-0018-US,1655,11/04/1994 |
| 1857 | hu00751458 | 568750 | IB:413486.3 | MMLR3DT01 | PD-0057-P,592,10/26/1995\|PD-0057-US,592,10/25/1996 |
| 1858 | hu00960726 | 1103 | IB:413486.3 | U937NOT01 | PD-0001-3-US,861,02/14/1994\|PD-0001-US,861,07/17/1992\|PD- |
| | | | | | 0001-1-PCT,1013,07/16/1993\|PD-0001-1-US,1013,11/19/1992\|PD- |
| | | | | | 0001-3-US,1013,02/14/1994 |
| 1859 | hu00610689 | 993 | IB:413486.3 | U937NOT01 | PD-0001-1-PCT,908,07/16/1993\|PD-0001-1-US,908,11/19/1992 |
| 1860 | hu00557338 | 36161 | IB:413486.3 | HUVENOB01 | PD-0003-1-US,3227,02/17/1994\|PD-0003-2-US,3408,04/28/1994\|PD |
| | | | | | 0003-3-US,687,08/26/1994 |
| 1861 | hu00357907 | 42068 | IB:413486.3 | TBLYNOT01 | PD-0007-US,1266,03/22/1994 |
| 1862 | hu00694950 | 46272 | IB:413486.3 | CORNNOT01 | PD-0006-US,615,03/31/1994 |
| 1863 | hu00535094 | 1471176 | IB:274440.1 | LUNGTUT03 | PD-0189-US,1296,06/19/1997\|PD-0189-P,1316,06/20/1996 |
| 1864 | hu00343557 | 911228 | IB:023525.10 | STOMNOT02 | PD-0155-P,499,04/26/1996\|PD-0155-US,499,04/25/1997 |
| 1865 | hu00367821 | 168804 | IB:023525.10 | LIVRNOT01 | PD-0064-P,925,10/10/1995\|PD-0064-US,925,10/09/1996 |
| 1866 | hu00475174 | 900839 | IB:023525.10 | BRSTTUT03 | PD-0143-1-US,1030,10/01/1997\|PD-0143-US,1030,03/20/1997\|PD- |
| | | | | | 0133-P,5240,03/20/1996 |
| 1867 | hu00165856 | 3897459 | IB:023525.10 | TLYMNOT05 | PD-0364-US,3132,04/30/1998 |
| 1868 | hu00177432 | 1840483 | IB:023525.10 | EOSITXT01 | PR-0011-P,1246,08/29/1996\|PR-0011-US,1246,08/29/1997 |
| 1869 | hu00309327 | 899181 | IB:023525.10 | BRSTTUT03 | PD-0143-1-US,361,10/01/1997\|PD-0143-US,361,03/20/1997\|PD- |
| | | | | | 0133-P,4571,03/20/1996 |
| 1870 | rat00077183 | 701024520 | ZS:221237.4 | RAFANOT02 | PZ-0101-P,828,12/10/1998 |
| 1871 | rat00174770 | 700420532 | ZS:221237.4 | RAHANOT01 | PZ-0040-US,585,10/08/1998\|PZ-0041-P,585,10/09/1997 |
| 1872 | rat00181648 | 700930974 | ZS:221237.4 | RALINON03 | PZ-0075-US,1146,03/09/1999\|PZ-0075-P,206,03/10/1998 |
| 1873 | rat00231043 | 700607265 | ZS:221237.4 | RALINOH01 | PZ-0053-P,243,12/12/1997\|PZ-0053-US,243,12/11/1998 |
| 1874 | rat00241986 | 701246834 | ZS:221237.4 | RALINOH02 | PZ-0091-P,2558,10/27/1998 |
| 1875 | rat00204820 | 700609603 | ZS:221237.4 | RALINOH01 | PZ-0053-P,738,12/12/1997\|PZ-0053-US,738,12/11/1998 |
| 1876 | rat00211971 | 701396879 | ZS:221237.4 | RALITXT14 | PZ-0094-P,3042,09/11/1998 |
| 1877 | rat00156450 | 701480622 | ZS:221237.4 | RALITXT42 | PZ-0109-P,592,02/11/1999 |
| 1878 | rat00058253 | 701343245 | ZS:221237.4 | RALITXT11 | PZ-0094-P,878,09/11/1998 |
| 1879 | hu01333685 | 6554941 | LG:023525.11 | BRAFNON02 | |
| 1880 | hu01102315 | 6559814 | LG:023525.11 | BRAFNON02 | |
| 1881 | rat00212947 | 700483216 | ZS:220065.1 | RALINON03 | PZ-0059-P,1418,01/16/1998\|PZ-0059-US,1418,01/12/1999 |
| 1882 | rat00209119 | 700483123 | ZS:220065.1 | RALINON03 | PZ-0059-P,1358,01/16/1998\|PZ-0059-US,1358,01/12/1999 |
| 1883 | rat00093295 | 700780819 | ZS:220065.2 | RABSNOT02 | PZ-0057-P,130,01/16/1998\|PZ-0057-US,130,01/11/1999 |
| 1884 | rat00083975 | 701485977 | ZS:220065.2 | RALITXT50 | PZ-0109-P,1659,02/11/1999 |
| 1885 | rat00139739 | 701318240 | ZS:220065.2 | RALITXT06 | PZ-0094-P,593,09/11/1998 |
| 1886 | rat00194974 | 701258865 | ZS:220065.2 | RALITXT03 | PZ-0096-P,977,10/09/1998 |
| 1887 | rat00002544 | 700314533 | ZS:220065.2 | RAESNOT01 | PZ-0032-P,479,10/08/1997\|PZ-0032-US,479,10/07/1998 |
| 1888 | rat00016900 | 700435941 | ZS:220065.2 | RAHLNOT02 | PZ-0047-P,430,11/06/1997\|PZ-0047-US,430,11/03/1998 |
| 1889 | rat00204309 | 700313454 | ZS:220065.2 | RAESNOT01 | PZ-0032-P,107,10/08/1997\|PZ-0032-US,107,10/07/1998 |
| 1890 | rat00181530 | 700938719 | ZS:220065.2 | RALINON03 | PZ-0075-P,3673,03/10/1998\|PZ-0075-US,4613,03/09/1999 |
| 1891 | rat00161335 | 700420843 | ZS:220065.2 | RAHANOT01 | PZ-0040-US,699,10/08/1998\|PZ-0041-P,699,10/09/1997 |
| 1892 | rat00190334 | 701275345 | ZS:220065.2 | RABFNON02 | PZ-0097-P,733,11/12/1998 |
| 1893 | rat00010973 | 700778749 | ZS:220065.2 | RAPINOT02 | PZ-0065-P,1085,02/11/1998\|PZ-0065-US,1085,02/10/1999 |
| 1894 | rat00254820 | 701394518 | ZS:220065.2 | RALITXT14 | PZ-0094-P,2653,09/11/1998 |
| 1895 | rat00013368 | 701879481 | ZS:220065.2 | RALITXS02 | PZ-0133-P,720,08/10/1999 |
| 1896 | rat00061429 | 700939406 | ZS:220065.2 | RALINON07 | PZ-0075-US,246,03/09/1999\|PZ-0079-P,246,04/08/1998 |
| 1897 | rat00240367 | 701909492 | ZS:220065.2 | RABYUNN02 | PZ-0130-P,238,06/29/1999 |
| 1898 | rat00006187 | 700780985 | ZS:220065.2 | RABSNOT02 | PZ-0057-P,211,01/16/1998\|PZ-0057-US,211,01/11/1999 |
| 1899 | rat00099014 | 701318033 | ZS:220065.2 | RALITXT06 | PZ-0094-P,524,09/11/1998 |
| 1900 | rat00219642 | 700525373 | ZS:220065.2 | RABMNOT01 | PZ-0022-US,1386,09/01/1998\|PZ-0051-P,1386,12/12/1997 |
| 1901 | hu01127888 | 5920416 | LG:023525.7 | BRAIFET02 | |
| 1902 | hu01039949 | 5885823 | LG:023525.7 | LIVRNON08 | |
| 1903 | hu01217791 | 5881319 | LG:023525.7 | LIVRNON08 | |
| 1904 | hu01202771 | 6270301 | LG:023525.7 | BRAIFEN03 | |
| 1905 | hu01303061 | 6271586 | LG:023525.7 | BRAIFEN03 | |
| 1906 | hu00983650 | 6552641 | LG:023525.7 | BRAFNON02 | |
| 1907 | hu01326455 | 5891134 | LG:023525.7 | LIVRNON08 | |
| 1908 | hu01279665 | 6116862 | LG:023525.8 | SINITMT04 | |
| 1909 | hu00989424 | 6424889 | LG:023525.8 | BRSTUNT01 | |
| 1910 | hu00992872 | 5616328 | LG:023525.8 | THYMNOR02 | |
| 1911 | hu01163899 | 5990859 | LG:023525.8 | FTUBTUT02 | |
| 1912 | hu01187866 | 6598719 | LG:023525.8 | ARTANOT06 | |
| 1913 | hu01309899 | 6379254 | LG:023525.8 | FIBAUNT01 | |
| 1914 | hu01100346 | 3760758 | LG:023525.8 | BRAHDIT03 | |
| 1915 | hu01229210 | 5839547 | LG:023525.8 | FIBRUNT02 | |
| 1916 | hu01333503 | 4515874 | LG:023525.8 | SINJNOT03 | |
| 1917 | hu01143532 | 5476167 | LG:023525.8 | FIBPFEN06 | |
| 1918 | hu01090190 | 6424790 | LG:023525.8 | BRSTUNT01 | |
| 1919 | hu01142734 | 6370928 | LG:023525.8 | ENDIUNT01 | |
| 1920 | hu01341626 | 6178092 | LG:023525.8 | BMARUNT02 | |
| 1921 | hu01047660 | 5475523 | LG:023525.8 | FIBPFEN06 | |
| 1922 | hu01040569 | 6496850 | LG:023525.8 | COLNNOT41 | |
| 1923 | hu01118167 | 5894616 | LG:023525.8 | BRAYDIN03 | |
| 1924 | hu01341962 | 5474001 | LG:023525.8 | FIBPFEN06 | |
| 1925 | hu01076606 | 6065772 | LG:023525.8 | BRAENOT02 | |
| 1926 | hu01246553 | 6152091 | LG:023525.8 | ENDMUNT04 | |
| 1927 | hu01308294 | 6125646 | LG:023525.8 | BRAHNON05 | |
| 1928 | hu01253673 | 6264310 | LG:023525.8 | MCLDTXN03 | |
| 1929 | hu01018949 | 6538510 | LG:023525.8 | OVARDIN02 | |
| 1930 | hu01295952 | 6262111 | LG:023525.8 | MCLDTXN03 | |
| 1931 | hu01186387 | 6190138 | LG:023525.8 | MUSCDIN06 | |
| 1932 | hu01292741 | 5941339 | LG:236453.4 | COLADIT05 | |
| 1933 | hu01133657 | 3903719 | LG:236453.4 | LUNGNON03 | |
| 1934 | hu00995052 | 6026458 | LG:236453.9 | TESTNOT11 | |
| 1935 | hu01265696 | 6113765 | LG:037054.5 | SINITMT04 | |
| 1936 | hu00990768 | 6262544 | LG:037054.5 | MCLDTXN03 | |
| 1937 | hu01317320 | 6425437 | LG:037054.5 | BRSTUNT01 | |
| 1938 | hu01242531 | 6211146 | LG:037054.5 | ARTANOT06 | |
| 1939 | hu01037206 | 6114029 | LG:037054.5 | SINITMT04 | |
| 1940 | hu01331119 | 6553732 | LG:037054.5 | BRAFNON02 | |
| 1941 | hu01052058 | 6323421 | LG:037054.5 | LUNGDIN02 | |
| 1942 | hu01113646 | 6296822 | LG:037054.5 | BRARTUT04 | |
| 1943 | hu01271048 | 6606417 | LG:037054.5 | UTREDIT07 | |
| 1944 | hu01204349 | 6496467 | LG:037054.5 | COLNNOT41 | |
| 1945 | hu01252447 | 6518170 | LG:037054.5 | BRAFTDT02 | |
| 1946 | hu01145870 | 6353795 | LG:037054.5 | LUNGDIS03 | |
| 1947 | hu01048355 | 6604422 | LG:037054.5 | UTREDIT07 | |
| 1948 | hu01314199 | 6386936 | LG:037054.5 | FIBRUNT02 | |
| 1949 | hu01329725 | 6130913 | LG:037054.5 | MCLDTXN03 | |
| 1950 | hu01317971 | 4519218 | LG:037054.5 | SINJNOT03 | |
| 1951 | hu01254829 | 6435737 | LG:037054.5 | LUNGNON07 | |
| 1952 | hu01188249 | 5475918 | LG:037054.5 | FIBPFEN06 | |
| 1953 | hu01258593 | 6542807 | LG:037054.5 | LNODNON02 | |
| 1954 | hu01208697 | 6317860 | LG:037054.5 | LUNGDIN02 | |
| 1955 | hu00979522 | 3023731 | LG:037054.5 | PROSDIN01 | |
| 1956 | hu01322638 | 6484989 | LG:037054.9 | MIXDUNB01 | |
| 1957 | hu00579220 | 1467992 | IB:815336.1 | PANCTUT02 | PD-0191-P,617,06/21/1996\|PD-0191-US,617,06/19/1997 |
| 1958 | hu00278027 | 908408 | IB:023525.6 | COLNNOT09 | PD-0129-P,398,03/19/1996\|PD-0129-US,398,03/18/1997 |
| 1959 | hu00150005 | 487818 | IB:023525.8 | HNT2AGT01 | |
| 1960 | hu00435663 | 490900 | IB:023525.8 | HNT2AGT01 | |
| 1961 | rat00104762 | 700273719 | ZS:275389.1 | RASJNOT01 | PZ-0039-P,1058,10/08/1997\|PZ-0039-US,1058,10/05/1998 |
| 1962 | hu01179863 | 3444223 | LG:413486.1 | BRONDIT01 | |
| 1963 | hu01016211 | 5989011 | LG:413486.1 | MCLDTXT02 | |
| 1964 | hu01003305 | 5872059 | LG:413486.1 | COLTDIT04 | |
| 1965 | hu00977589 | 6095094 | LG:413486.1 | THP1TXT03 | |
| 1966 | hu01130225 | 6093023 | LG:413486.1 | THP1TXT03 | |
| 1967 | hu01205304 | 6075596 | LG:413486.1 | UTREDIT09 | |
| 1968 | hu01017422 | 6419675 | LG:413486.1 | BRSTUNT01 | |
| 1969 | hu01200377 | 6209547 | LG:413486.1 | ARTANOT06 | |
| 1970 | hu01067225 | 6405076 | LG:413486.1 | UTREDIT10 | |
| 1971 | hu01084631 | 6078425 | LG:413486.1 | UTREDIT09 | |
| 1972 | hu01047466 | 5981860 | LG:413486.1 | MCLDTXT02 | |
| 1973 | hu01343872 | 5983358 | LG:413486.1 | MCLDTXT02 | |
| 1974 | hu01077823 | 5979930 | LG:413486.1 | MCLDTXT02 | |
| 1975 | hu01326827 | 6261877 | LG:413486.1 | BRSTUNT01 | |
| 1976 | hu01348805 | 6375405 | LG:413486.1 | FIBAUNT01 | |
| 1977 | hu01090839 | 5981662 | LG:413486.1 | MCLDTXT02 | |
| 1978 | hu01090596 | 6564362 | LG:413486.1 | MCLDTXT04 | |
| 1979 | hu01325858 | 6422216 | LG:413486.1 | BRSTUNT01 | |
| 1980 | hu01279463 | 3902250 | LG:413486.1 | LUNGNON03 | |
| 1981 | hu00974569 | 5953976 | LG:413486.1 | SKINTDT01 | |
| 1982 | hu01092102 | 6133582 | LG:413486.1 | BMARTXT02 | |
| 1983 | hu01335302 | 3166285 | LG:413486.1 | TLYMTXT04 | |
| 1984 | hu01172132 | 6154521 | LG:413486.1 | ENDMUNT04 | |
| 1985 | hu01027317 | 5564143 | LG:413486.1 | BRSTDIT01 | |
| 1986 | hu01312473 | 6131318 | LG:413486.1 | BMARTXT02 | |
| 1987 | hu01253290 | 6114440 | LG:413486.1 | SINITMT04 | |
| 1988 | hu01003675 | 6564795 | LG:413486.1 | MCLDTXT04 | |
| 1989 | hu01124392 | 6131579 | LG:413486.1 | BMARTXT02 | |
| 1990 | hu01311863 | 6299814 | LG:413486.1 | UTREDIT07 | |
| 1991 | hu01192335 | 6151928 | LG:413486.1 | ENDMUNT04 | |
| 1992 | hu01327723 | 6388403 | LG:413486.1 | COLNDIC01 | |
| 1993 | hu01287332 | 6150440 | LG:413486.1 | ENDMUNT04 | |
| 1994 | hu01045376 | 3165833 | LG:413486.1 | TLYMTXT04 | |
| 1995 | hu01017559 | 6396535 | LG:413486.1 | UTRENOT10 | |
| 1996 | hu01055819 | 6361347 | LG:413486.1 | CONFNOT09 | |
| 1997 | hu01070968 | 5984828 | LG:413486.1 | MCLDTXT02 | |
| 1998 | hu01326993 | 5987020 | LG:413486.1 | MCLDTXT02 | |
| 1999 | hu01196696 | 6408470 | LG:413486.1 | UTREDIT10 | |
| 2000 | hu01168994 | 6270190 | LG:413486.1 | BRAIFEN03 | |
| 2001 | hu01071100 | 5983366 | LG:413486.1 | MCLDTXT02 | |
| 2002 | hu01071102 | 6422308 | LG:413486.1 | BRSTUNT01 | |
| 2003 | hu01014015 | 5908769 | LG:413486.1 | BRAIFEN05 | |
| 2004 | hu01141219 | 5658363 | LG:413486.1 | BSCNNOT03 | |
| 2005 | hu01317018 | 6454559 | LG:413486.1 | COLNDIC01 | |
| 2006 | hu01117390 | 5475468 | LG:413486.1 | FIBPFEN06 | |
| 2007 | hu01062077 | 6420636 | LG:413486.1 | BRSTUNT01 | |
| 2008 | hu01216176 | 6538777 | LG:413486.1 | OVARDIN02 | |
| 2009 | hu01155094 | 3444239 | LG:413486.1 | BRONDIT01 | |
| 2010 | hu01022992 | 1896359 | LG:413486.1 | THP1TXT04 | |
| 2011 | hu01208276 | 6112991 | LG:413486.1 | SINITMT04 | |
| 2012 | hu01150884 | 5986966 | LG:413486.1 | MCLDTXT02 | |
| 2013 | hu01282617 | 5809528 | LG:413486.1 | KIDCTMT02 | |
| 2014 | hu01331676 | 5987770 | LG:413486.1 | MCLDTXT02 | |
| 2015 | hu01148923 | 5988150 | LG:413486.1 | MCLDTXT02 | |
| 2016 | hu01179335 | 6561673 | LG:413486.1 | MCLDTXT04 | |
| 2017 | hu01132985 | 6425449 | LG:413486.1 | BRSTUNT01 | |
| 2018 | hu01199336 | 6422516 | LG:413486.1 | BRSTUNT01 | |
| 2019 | hu01185693 | 4516438 | LG:413486.1 | SINJNOT03 | |
| 2020 | hu00969471 | 6514835 | LG:413486.1 | THYMDIT01 | |
| 2021 | hu00975597 | 1895668 | LG:413486.1 | THP1TXT04 | |
| 2022 | hu01114017 | 6170203 | LG:413486.1 | UTRSTDT01 | |
| 2023 | hu01013290 | 6402652 | LG:413486.1 | UTRENOT10 | |
| 2024 | hu01224206 | 6095029 | LG:413486.1 | THP1TXT03 | |
| 2025 | hu01211615 | 5879126 | LG:413486.1 | BRAUNOT01 | |
| 2026 | hu01141146 | 4123126 | LG:413486.1 | BRSTTUT25 | |
| 2027 | hu01284800 | 5563507 | LG:413486.1 | BRSTDIT01 | |
| 2028 | hu01207517 | 6123211 | LG:413486.1 | BRAHNON05 | |
| 2029 | hu01247630 | 6371816 | LG:413486.1 | FIBAUNT02 | |
| 2030 | hu01060945 | 3158120 | LG:413486.1 | TLYMTXT03 | |
| 2031 | hu01332655 | 6402620 | LG:413486.1 | UTRENOT10 | |
| 2032 | hu01015795 | 5954076 | LG:413486.1 | SKINTDT01 | |
| 2033 | hu01309880 | 5870789 | LG:413486.1 | COLTDIT04 | |
| 2034 | hu01346890 | 6419582 | LG:413486.1 | BRSTUNT01 | |
| 2035 | hu01182595 | 6497557 | LG:413486.1 | COLNNOT41 | |
| 2036 | rat00225961 | 700269380 | ZS:202666.1 | RAADNOT03 | PZ-0011-US,1736,07/30/1998\|PZ-0031-P,543,10/10/1997 |
| 2037 | rat00027224 | 700061367 | ZS:202666.2 | RAKINOT02 | PZ-0002-P,1040,06/13/1997\|PZ-0002-US,1782,06/09/1998 |
| 2038 | rat00216641 | 700484428 | ZS:202666.2 | RALINON03 | PZ-0059-P,1878,01/16/1998\|PZ-0059-US,1878,01/12/1999 |
| 2039 | hu01057340 | 6512973 | LG:952911.1 | THYMDIT01 | |
| 2040 | hu00914886 | 30396 | IB:236453.10 | THP1NOB01 | PD-0001-3-US,4967,02/14/1994\|PD-0001-4-US,5137,07/28/1994 |
| 2041 | hu00582409 | 2113 | IB:236453.10 | U937NOT01 | PD-0001-1-PCT,1920,07/16/1993\|PD-0001-1- |
| | | | | | US,1920,11/19/1992\|PD-0001-3-US,1920,02/14/1994 |
| 2042 | hu00193205 | 2741933 | IB:236453.10 | BRSTTUT14 | PD-0320-P,3051,02/13/1997\|PD-0320-US,3051,02/11/1998 |
| 2043 | hu00691687 | 100690 | IB:236453.10 | ADRENOT01 | PD-0016-US,1018,10/14/1994 |
| 2044 | hu00601763 | 2304342 | IB:236453.10 | BRSTNOT05 | PD-0292-P,4070,12/19/1996\|PD-0292-US,4070,12/18/1997 |
| 2045 | hu00459499 | 1807710 | IB:236453.6 | SINTNOT13 | PD-0234-P,1414,08/28/1996 |
| 2046 | hu00772054 | 1295954 | IB:236453.6 | PGANNOT03 | PD-0165-P,1325,05/28/1996\|PD-0165-US,1325,05/23/1997 |
| 2047 | hu00095625 | 52759 | IB:236453.8 | | PD-0008-US,259,06/27/1994 |
| 2048 | hu00128036 | 2797366 | IB:236453.8 | NPOLNOT01 | PD-0340-P,1113,03/28/1997\|PD-0340-US,1113,03/27/1998 |
| 2049 | hu00870833 | 2906493 | IB:236453.8 | THYMNOT05 | PD-0393-P,984,07/02/1997\|PD-0393-US,984,06/24/1998 |
| 2050 | hu00708205 | 2729355 | IB:236453.9 | OVARTUT05 | PD-0313-P,3804,01/22/1997\|PD-0313-US,3804,01/22/1998 |
| 2051 | rat00154045 | 700910295 | ZS:259301.1 | RAXSNOT02 | PZ-0100-P,2066,11/12/1998 |
| 2052 | hu00875504 | 782418 | IB:037054.5 | MYOMNOT01 | PD-0113-P,920,02/27/1996\|PD-0113-US,920,02/27/1997 |
| 2053 | hu00061898 | 1430906 | IB:037054.5 | SINTBST01 | PD-0209-P,2519,07/23/1996\|PD-0209-US,2519,07/22/1997 |
| 2054 | hu00853194 | 2455564 | IB:037054.6 | ENDANOT01 | PD-0299-P,1815,12/16/1996\|PD-0299-US,1815,12/04/1997 |
| 2055 | hu00890790 | 3086914 | IB:037054.6 | HEAONOT03 | PD-0352-P,915,04/18/1997\|PD-0352-US,915,04/24/1998 |
| 2056 | hu00760437 | 1634012 | IB:037054.7 | COLNNOT19 | PD-0206-P,635,08/01/1996\|PD-0206-US,635,08/01/1997 |
| 2057 | hu00890998 | 1216570 | IB:037054.7 | BRSTTUT01 | PD-0157-P,3433,04/26/1996\|PD-0157-US,3433,04/25/1997 |
| 2058 | hu00152759 | 2841268 | IB:037054.7 | DRGLNOT01 | PD-0337-P,1648,03/18/1997\|PD-0337-US,1648,03/05/1998 |
| 2059 | hu00748297 | 830485 | IB:037054.8 | PROSTUT04 | PD-0117-P,2114,02/26/1996\|PD-0117-US,2114,02/26/1997\|PD-0197 |
| | | | | | US,538,06/23/1997 |
| 2060 | hu00958835 | 233757 | IB:037054.8 | SINTNOT02 | PD-0023-US,524,02/22/1995 |
| 2061 | hu00797565 | 57367 | IB:037054.8 | | PD-0009-US,131,07/08/1994 |
| 2062 | hu00023242 | 2194388 | IB:037054.8 | THYRTUT03 | PD-0271-P,1679,10/31/1996 |
| 2063 | hu00219001 | 766272 | IB:037054.8 | LUNGNOT04 | PD-0121-US,2271,02/28/1997\|PD-0140-P,824,04/10/1996 |
| 2064 | hu00270032 | 750798 | IB:037054.8 | BRAITUT01 | PD-0104-P,2049,01/30/1996 |
| 2065 | hu00119012 | 935410 | IB:037054.8 | CERVNOT01 | PD-0128-P,1739,03/22/1996 |
| 2066 | hu00160405 | 825399 | IB:037054.8 | PROSNOT06 | PD-0117-P,611,02/26/1996\|PD-0117-US,611,02/26/1997 |
| 2067 | hu00190208 | 601853 | IB:037054.8 | BRSTNOT02 | PD-0061-P,1466,11/15/1995\|PD-0061-US,1466,11/15/1996 |
| 2068 | hu00626332 | 2522237 | IB:037054.8 | BRAITUT21 | PD-0304-P,1507,01/28/1997\|PD-0304-US,1507,01/28/1998 |
| 2069 | hu00100687 | 1333153 | IB:037054.8 | PANCNOT07 | PD-0172-P,1431,05/23/1996\|PD-0172-US,1431,05/22/1997 |
| 2070 | hu00320665 | 2085612 | IB:037054.8 | PANCNOT04 | PD-0291-P,185,12/17/1996 |
| 2071 | hu00239361 | 935740 | IB:037054.8 | CERVNOT01 | PD-0128-P,1820,03/22/1996 |
| 2072 | hu00920049 | 523577 | IB:037054.8 | MMLR2DT01 | PD-0044-P,2621,10/02/1995\|PD-0044-US,2621,10/02/1996 |
| 2073 | hu00524608 | 866666 | IB:037054.8 | BRAITUT03 | PD-0126-1-US,3167,11/25/1997\|PD-0126-P,3167,03/08/1996\|PD- |
| | | | | | 0126-US,3167,03/07/1997 |
| 2074 | hu00364660 | 2951387 | IB:037054.8 | KIDNFET01 | PD-0339-P,1486,03/14/1997\|PD-0339-US,1486,03/12/1998 |
| 2075 | hu00845262 | 1430663 | IB:037054.8 | SINTBST01 | PD-0209-P,2459,07/23/1996\|PD-0209-US,2459,07/22/1997 |
| 2076 | hu00633709 | 1361476 | IB:037054.8 | LUNGNOT12 | PD-0188-P,571,06/19/1997\|PD-0188-US,571,06/18/1997 |
| 2077 | hu00495146 | 1560126 | IB:037054.8 | SPLNNOT04 | PD-0190-US,2155,06/19/1997\|PD-0215-P,575,07/30/1996 |
| 2078 | hu00913556 | 744459 | IB:037054.8 | BRAITUT01 | PD-0104-P,246,01/30/1996 |
| 2079 | hu00428818 | 1797747 | IB:037054.8 | PROSTUT05 | PD-0119-1-US,3954,09/30/1997 |
| 2080 | hu00565575 | 2183401 | IB:037054.8 | SININOT01 | PD-0267-P,1318,10/31/1996\|PD-0267-US,1318,10/29/1997 |
| 2081 | hu00827367 | 603570 | IB:037054.8 | BRSTTUT01 | PD-0061-P,1987,11/15/1995\|PD-0061-US,1987,11/15/1996 |
| 2082 | hu00786562 | 942875 | IB:037054.8 | ADRENOT03 | PD-0144-P,857,04/10/1996\|PD-0144-US,857,04/10/1997 |
| 2083 | hu00228878 | 1457010 | IB:037054.8 | COLNFET02 | PD-0168-US,2087,05/21/1997\|PD-0195-P,536,06/19/1996 |
| 2084 | hu00356687 | 2212106 | IB:037054.8 | SINTFET03 | PD-0268-P,2013,10/31/1996\|PD-0268-US,2013,10/30/1997 |
| 2085 | hu00878932 | 2451987 | IB:037054.8 | ENDANOT01 | PD-0299-P,872,12/16/1996\|PD-0299-US,872,12/04/1997 |
| 2086 | hu00501343 | 1419958 | IB:037054.8 | KIDNNOT09 | PD-0186-P,704,06/25/1996\|PD-0186-US,704,06/24/1997 |
| 2087 | hu00531225 | 1903227 | IB:037054.8 | OVARNOT07 | PD-0249-P,2163,10/01/1996 |
| 2088 | hu00100738 | 35143 | IB:037054.8 | HUVENOB01 | PD-0003-3-US,107,08/26/1994\|PD-0003-1-US,2691,02/17/1994\|PD- |
| | | | | | 0003-2-US,2828,04/28/1994 |
| 2089 | hu00114571 | 1647305 | IB:037054.8 | PROSTUT09 | PD-0199-P,2713,07/30/1996 |
| 2090 | hu00161624 | 2057559 | IB:037054.8 | BEPINOT01 | PD-0182-P,1446,09/25/1996 |
| 2091 | hu00161626 | 2079008 | IB:037054.8 | ISLTNOT01 | PD-0259-P,3003,10/28/1996\|PD-0259-US,3003,10/28/1997 |
| 2092 | hu00534136 | 2174488 | IB:037054.8 | ENDCNOT03 | PD-0279-P,2213,11/22/1996\|PD-0279-US,2213,11/07/1997 |
| 2093 | hu00107681 | 1806270 | IB:037054.8 | SINTNOT13 | PD-0234-P,926,08/28/1996 |
| 2094 | hu00257711 | 2472046 | IB:037054.8 | THP1NOT03 | PD-0286-P,882,11/25/1996\|PD-0286-US,882,11/04/1997 |
| 2095 | hu00237192 | 1493214 | IB:037054.8 | PROSNON01 | PD-0198-P,425,07/29/1996\|PD-0198-US,5032,07/28/1997 |
| 2096 | hu00770272 | 853974 | IB:037054.8 | NGANNOT01 | PD-0123-US,787,02/28/1997\|PD-0125-P,787,03/07/1996 |
| 2097 | hu00409950 | 900309 | IB:037054.8 | BRSTTUT03 | PD-0133-P,5078,03/20/1996\|PD-0143-1-US,868,10/01/1997\|PD- |
| | | | | | 0143-US,868,03/20/1997 |
| 2098 | hu00467172 | 3868358 | IB:037054.8 | BMARNOT03 | PD-0218-1-US,1748,03/12/1998 |
| 2099 | hu00609932 | 2914677 | IB:037054.8 | THYMFET03 | PD-0345-P,295,03/21/1997\|PD-0345-US,295,03/18/1998 |
| 2100 | hu00318990 | 2322455 | IB:037054.8 | OVARNOT02 | PD-0293-P,1764,12/13/1996\|PD-0045-1-US,3591,11/10/1997 |
| 2101 | hu00156990 | 966061 | IB:037054.8 | BRSTNOT05 | PD-0133-P,2807,03/20/1996\|PD-0133-US,2807,03/20/1997 |
| 2102 | hu00791682 | 1664515 | IB:037054.8 | BRSTNOT09 | PD-0219-P,1237,07/26/1996 |
| 2103 | hu00383668 | 825238 | IB:037054.8 | PROSNOT06 | PD-0117-P,571,02/26/1996\|PD-0117-US,571,02/26/1997 |
| 2104 | hu00956473 | 1556945 | IB:037054.8 | BLADTUT04 | PD-0203-P,4234,08/01/1996\|PD-0203-US,4234,08/01/1997 |
| 2105 | hu00858068 | 2602985 | IB:037054.8 | UTRSNOT10 | PD-0316-P,1679,01/29/1997\|PD-0316-US,1679,01/26/1998 |
| 2106 | hu00774953 | 1680495 | IB:037054.8 | STOMFET01 | PD-0217-P,1125,07/30/1996 |
| 2107 | hu00759728 | 1794004 | IB:037054.8 | PROSTUT05 | PD-0119-1-US,2498,09/30/1997 |
| 2108 | hu00889195 | 83448 | IB:037054.8 | HUVESTB01 | PD-0003-3-US,6538,08/26/1994 |
| 2109 | hu00665977 | 1267536 | IB:037054.8 | BRAINOT09 | PD-0163-P,651,05/21/1996\|PD-0163-US,651,05/20/1997 |
| 2110 | hu00520221 | 2106813 | IB:037054.8 | BRAITUT03 | PD-0126-1-US,4555,11/25/1997\|PD-0275-P,8040,12/06/1996 |
| 2111 | hu00731210 | 1510165 | IB:037054.8 | LUNGNOT14 | PD-0208-P,828,07/24/1996\|PD-0208-US,828,07/23/1997 |
| 2112 | hu00005443 | 1856492 | IB:037054.8 | PROSNOT18 | PD-0233-P,245,09/05/1996\|PD-0233-US,245,09/05/1997 |
| 2113 | hu00641224 | 2266659 | IB:037054.8 | UTRSNOT02 | PD-0288-P,464,12/18/1996\|PD-0288-US,464,12/16/1997 |
| 2114 | hu00117253 | 1558625 | IB:037054.8 | SPLNNOT04 | PD-0215-P,115,07/30/1996\|PD-0190-US,1695,06/19/1997 |
| 2115 | hu00920305 | 233733 | IB:037054.8 | SINTNOT02 | PD-0023-US,518,02/22/1995 |
| 2116 | hu00363233 | 1551996 | IB:037054.8 | PROSNOT06 | PD-0199-P,5710,07/30/1996 |
| 2117 | hu00222753 | 1986820 | IB:037054.8 | LUNGAST01 | PD-0120-US,4332,02/28/1997\|PD-0247-P,986,09/27/1996 |
| 2118 | hu00394401 | 877233 | IB:037054.8 | LUNGAST01 | PD-0120-US,1823,02/28/1997\|PD-0124-P,1823,03/13/1996 |
| 2119 | hu00282154 | 2256047 | IB:037054.8 | OVARTUT01 | PD-0283-P,2267,11/21/1996\|PD-0112-1-US,4127,11/14/1997 |
| 2120 | hu00183253 | 1911310 | IB:037054.8 | CONNTUT01 | PD-0252-P,1327,10/01/1996\|PD-0254-P,1327,10/04/1996 |
| 2121 | hu00278213 | 678398 | IB:037054.8 | CRBLNOT01 | PD-0093-P,1900,12/18/1995\|PD-0089-US,3596,11/27/1996 |
| 2122 | hu00091924 | 1804361 | IB:037054.8 | SINTNOT13 | PD-0234-P,316,08/28/1996 |
| 2123 | hu00474271 | 935092 | IB:037054.8 | CERVNOT01 | PD-0128-P,1668,03/22/1996 |
| 2124 | hu00260575 | 1905732 | IB:037054.8 | OVARNOT07 | PD-0249-P,3110,10/01/1996 |
| 2125 | hu00260692 | 1614400 | IB:037054.8 | BRAITUT12 | PD-0204-P,3628,07/26/1996 |
| 2126 | hu00882413 | 35182 | IB:037054.8 | HUVENOB01 | PD-0003-3-US,135,08/26/1994\|PD-0003-1-US,2719,02/17/1994\|PD- |
| | | | | | 0003-2-US,2856,04/28/1994 |
| 2127 | hu00891127 | 3326065 | IB:037054.8 | HEAONOT04 | PD-0387-P,2164,07/03/1997\|PD-0387-US,2164,06/30/1998 |
| 2128 | hu00198146 | 582185 | IB:037054.8 | PROSNOT02 | PD-0086-P,121,10/27/1995\|PD-0086-US,121,10/21/1996 |
| 2129 | hu00791651 | 991896 | IB:037054.8 | COLNNOT11 | PD-0129-P,1641,03/19/1996\|PD-0129-US,1641,03/18/1997 |
| 2130 | hu00924863 | 1212159 | IB:037054.8 | BRSTTUT01 | PD-0157-P,1998,04/26/1996\|PD-0157-US,1998,04/25/1997 |
| 2131 | hu00491649 | 555259 | IB:037054.8 | SCORNOT01 | PD-0066-P,1199,10/31/1995 |
| 2132 | hu00739378 | 798805 | IB:037054.8 | BRAVTXT04 | PR-0005-P,22,01/10/1996\|PR-0005-US,22,01/10/1997 |
| 2133 | hu00478991 | 2626808 | IB:037054.8 | PROSTUT12 | PD-0314-US,2838,09/04/1997\|PD-0314-P,889,01/22/1997 |
| 2134 | hu00820521 | 2758919 | IB:037054.8 | THP1AZS08 | PD-0362-P,4800,05/01/1997\|PD-0362-US,4800,04/29/1998 |
| 2135 | hu00004088 | 1618796 | IB:037054.8 | BRAITUT12 | PD-0204-P,3315,07/26/1996 |
| 2136 | hu00019055 | 1640142 | IB:037054.8 | UTRSNOT06 | PD-0211-P,3626,07/25/1996\|PD-0211-US,3626,07/24/1997 |
| 2137 | hu00138630 | 3228228 | IB:037054.8 | COTRNOT01 | PD-0372-P,440,05/14/1997\|PD-0372-US,440,04/30/1998 |
| 2138 | hu00411573 | 685491 | IB:037054.8 | UTRSNOT02 | PD-0095-P,2151,12/29/1995\|PD-0095-US,2151,12/23/1996 |
| 2139 | hu00543181 | 1443405 | IB:037054.8 | THYRNOT03 | PD-0162-P,3455,05/29/1996\|PD-0162-US,3455,05/27/1997 |
| 2140 | hu00407552 | 1305226 | IB:037054.8 | PLACNOT02 | PD-0167-P,673,05/30/1996\|PD-0167-US,673,05/29/1997 |
| 2141 | hu00446342 | 751876 | IB:037054.8 | BRAITUT01 | PD-0104-P,2397,01/30/1996 |
| 2142 | hu00374090 | 3289296 | IB:037054.8 | BONRFET01 | PD-0368-P,374,05/28/1997\|PD-0368-US,374,05/05/1998 |
| 2143 | hu00065058 | 1555366 | IB:037054.8 | BLADTUT04 | PD-0203-P,3759,08/01/1996\|PD-0203-US,3759,08/01/1997 |
| 2144 | hu00484406 | 2324241 | IB:037054.8 | OVARNOT02 | PD-0293-P,2301,12/13/1996\|PD-0045-1-US,4128,11/10/1997 |
| 2145 | hu00346157 | 3201317 | IB:037054.8 | PENCNOT02 | PD-0375-P,813,06/02/1997\|PD-0375-US,813,05/08/1998 |
| 2146 | hu00428607 | 1379439 | IB:037054.8 | LUNGNOT10 | PD-0179-P,1315,05/13/1996\|PD-0179-US,1315,05/09/1997 |
| 2147 | hu00495415 | 2461925 | IB:037054.8 | THYRNOT08 | PD-0394-P,950,07/02/1997\|PD-0394-US,950,06/24/1998 |
| 2148 | hu00722593 | 902077 | IB:037054.8 | BRSTTUT03 | PD-0143-1-US,1590,10/01/1997\|PD-0143-US,1590,03/20/1997\|PD- |
| | | | | | 0133-P,5800,03/20/1996 |
| 2149 | hu00155683 | 1482170 | IB:037054.8 | CORPNOT02 | PD-0185-P,2094,06/26/1996\|PD-0185-US,2094,06/26/1997 |
| 2150 | hu00839215 | 586905 | IB:037054.8 | UTRSNOT01 | PD-0084-P,114,10/26/1995\|PD-0084-US,114,10/18/1996 |
| 2151 | hu00868081 | 2445442 | IB:037054.8 | THP1NOT03 | PD-0286-P,2185,11/25/1996\|PD-0286-US,2185,11/04/1997 |
| 2152 | hu01322291 | 5850349 | LG:236453.11 | BRAENOT04 | |
| 2153 | hu01053686 | 6520711 | LG:236453.11 | CONFTDT02 | |
| 2154 | hu00984752 | 5742854 | LG:236453.12 | LUNGNON03 | |
| 2155 | hu01121158 | 6589010 | LG:236453.12 | TLYMUNT03 | |
| 2156 | hu01069140 | 5679919 | LG:236453.12 | BRAENOT02 | |
| 2157 | hu01289279 | 1892562 | LG:236453.12 | THP1TXT04 | |
| 2158 | hu01220863 | 5985587 | LG:236453.12 | MCLDTXT02 | |
| 2159 | hu01044339 | 3780406 | LG:236453.12 | BRAHDIT04 | |
| 2160 | hu01286152 | 4205646 | LG:236453.12 | BRONNOT02 | |
| 2161 | hu01336363 | 6284143 | LG:236453.12 | SKINDIA01 | |
| 2162 | hu01118609 | 6259745 | LG:236453.12 | BMARTXT06 | |
| 2163 | hu00992223 | 6361023 | LG:236453.12 | CONFNOT09 | |
| 2164 | hu01142739 | 6517006 | LG:236453.12 | THYMDIT01 | |
| 2165 | hu01345188 | 6522491 | LG:236453.12 | CONFTDT02 | |
| 2166 | hu01199245 | 6496384 | LG:236453.12 | COLNNOT41 | |
| 2167 | hu01205260 | 5873053 | LG:236453.12 | COLTDIT04 | |
| 2168 | hu01232431 | 6360111 | LG:236453.12 | CONFNOT09 | |
| 2169 | hu01136250 | 5657335 | LG:236453.12 | BSCNNOT03 | |
| 2170 | hu01156768 | 6395718 | LG:236453.12 | UTRENOT10 | |
| 2171 | hu01020636 | 6260645 | LG:236453.12 | BMARTXT06 | |

TABLE 2

| SEQ ID NO | CLONE | TEMPLATE | START | STOP |
|---|---|---|---|---|
| 1 | 5564221 | LG:2020790.1 | 1 | 247 |
| 2 | 2266979 | IB:187861.1 | 1 | 241 |
| 3 | 6422174 | LG:2023386.5 | 1 | 507 |
| 4 | 6413044 | LG:2023386.5 | 1 | 134 |
| 5 | 6180012 | LG:2023386.5 | 1 | 279 |
| 6 | 5884355 | LG:2023386.5 | 1 | 210 |
| 7 | 6325092 | LG:2023386.5 | 1 | 260 |
| 8 | 6377218 | LG:2023386.5 | 2 | 117 |
| 9 | 5886010 | LG:2023386.5 | 1 | 254 |
| 10 | 6421181 | LG:2023386.5 | 12 | 181 |
| 11 | 6479549 | LG:2023386.5 | 1 | 540 |
| 12 | 5949878 | LG:2023386.5 | 1 | 337 |
| 13 | 5656380 | LG:2023386.5 | 1 | 267 |
| 14 | 6325013 | LG:2023386.5 | 4 | 60 |
| 15 | 6358853 | LG:2023386.5 | 21 | 131 |
| 16 | 5844535 | LG:2023386.5 | 1 | 259 |
| 17 | 5994013 | LG:1909275.1 | 1 | 315 |
| 18 | 6419669 | LG:1981103.3 | 1 | 545 |
| 19 | 3549771 | LG:1907955.5 | 1 | 247 |
| 20 | 6030694 | LG:1907955.5 | 1 | 239 |
| 21 | 4519787 | LG:1907955.5 | 1 | 239 |
| 22 | 6111313 | LG:1907955.5 | 2 | 306 |
| 23 | 4437887 | LG:1907955.5 | 2 | 251 |
| 24 | 5911025 | LG:1907955.5 | 2 | 292 |
| 25 | 6152806 | LG:1907955.5 | 1 | 288 |
| 26 | 6119359 | LG:1907955.5 | 1 | 457 |
| 27 | 5714759 | LG:1907955.5 | 1 | 289 |
| 28 | 6099952 | LG:1907955.5 | 5 | 270 |
| 29 | 6209977 | LG:1907955.5 | 4 | 156 |
| 30 | 3153065 | LG:1907955.5 | 2 | 168 |
| 31 | 4437787 | LG:1907955.5 | 3 | 259 |
| 32 | 4442819 | LG:1907955.5 | 1 | 285 |
| 33 | 6378670 | LG:1907955.5 | 1 | 307 |
| 34 | 6015470 | LG:1907955.5 | 1 | 266 |
| 35 | 4517716 | LG:1907955.5 | 6 | 279 |
| 36 | 6383466 | LG:1907955.5 | 1 | 291 |
| 37 | 6495789 | LG:1907955.5 | 1 | 333 |
| 38 | 5811756 | LG:1907955.5 | 1 | 317 |
| 39 | 3901675 | LG:1907955.5 | 1 | 277 |
| 40 | 523345 | IB:2056060.1 | 1 | 244 |
| 41 | 2024316 | IB:2097880.1 | 1 | 283 |
| 42 | 2663830 | IB:2097880.1 | 1 | 122 |
| 43 | 2069960 | IB:2097880.1 | 1 | 129 |
| 44 | 2850044 | IB:2097880.1 | 1 | 91 |
| 45 | 2484903 | IB:2097880.1 | 1 | 84 |
| 46 | 1493337 | IB:2097880.1 | 1 | 225 |
| 47 | 664658 | IB:2097880.1 | 1 | 106 |
| 48 | 386476 | IB:2097880.1 | 1 | 274 |
| 49 | 488003 | IB:2097880.1 | 1 | 252 |
| 50 | 2634306 | IB:2097880.1 | 1 | 251 |
| 51 | 2318093 | IB:2097880.1 | 1 | 261 |
| 52 | 611497 | IB:2097880.1 | 1 | 85 |
| 53 | 873135 | IB:2097880.1 | 1 | 73 |
| 54 | 2367584 | IB:2097880.1 | 1 | 229 |
| 55 | 57744 | IB:2097880.1 | 1 | 191 |
| 56 | 621001 | IB:2097880.1 | 1 | 145 |
| 57 | 1260150 | IB:2097880.1 | 1 | 245 |
| 58 | 2365110 | IB:2097880.1 | 1 | 227 |
| 59 | 2364628 | IB:2097880.1 | 1 | 231 |
| 60 | 2869942 | IB:2097880.1 | 1 | 254 |
| 61 | 2858353 | IB:2097880.1 | 1 | 267 |
| 62 | 3034616 | IB:2097880.1 | 1 | 209 |
| 63 | 621158 | IB:2097880.1 | 1 | 145 |
| 64 | 2632061 | IB:2097880.1 | 1 | 75 |
| 65 | 640040 | IB:2097880.1 | 1 | 266 |
| 66 | 2841649 | IB:2097880.1 | 1 | 232 |
| 67 | 6057485 | LG:1695209.1 | 1 | 232 |
| 68 | 4125481 | LG:1907955.3 | 1 | 271 |
| 69 | 2965517 | IB:2095468.1 | 1 | 272 |
| 70 | 6208681 | LG:1907955.4 | 1 | 264 |
| 71 | 6075669 | LG:1907955.4 | 1 | 308 |
| 72 | 6566164 | LG:1944384.1 | 1 | 475 |
| 73 | 6271522 | LG:1946261.1 | 1 | 449 |
| 74 | 5560063 | LG:2022353.5 | 1 | 261 |
| 75 | 5561495 | LG:2022353.5 | 1 | 248 |
| 76 | 6433941 | LG:2022353.5 | 1 | 452 |
| 77 | 6456026 | LG:2022353.5 | 3 | 575 |
| 78 | 4121704 | LG:2022353.5 | 1 | 269 |
| 79 | 6137123 | LG:2022353.5 | 1 | 292 |
| 80 | 5656338 | LG:2022353.5 | 1 | 279 |
| 81 | 6137155 | LG:2022353.5 | 1 | 296 |
| 82 | 5943751 | LG:2022353.5 | 1 | 288 |
| 83 | 6380182 | LG:2022353.5 | 10 | 173 |
| 84 | 6409908 | LG:2022353.5 | 1 | 64 |
| 85 | 5697069 | LG:2022353.5 | 1 | 232 |
| 86 | 3567727 | LG:2022353.5 | 1 | 167 |
| 87 | 6507225 | LG:2022353.5 | 1 | 437 |
| 88 | 6389422 | LG:2022353.5 | 1 | 302 |
| 89 | 5810417 | LG:2022353.5 | 1 | 279 |
| 90 | 6522760 | LG:2022353.5 | 1 | 529 |
| 91 | 5646107 | LG:2022353.5 | 1 | 181 |
| 92 | 6397064 | LG:2022353.5 | 1 | 180 |
| 93 | 5943406 | LG:1922100.1 | 1 | 144 |
| 94 | 700294566 | ZS:169425.2 | 1 | 280 |
| 95 | 700309980 | ZS:169425.2 | 1 | 264 |
| 96 | 700933938 | ZS:169425.2 | 1 | 261 |
| 97 | 701904240 | ZS:169425.2 | 1 | 174 |
| 98 | 701231886 | ZS:169425.2 | 1 | 226 |
| 99 | 700127612 | ZS:169425.2 | 1 | 260 |
| 100 | 701828746 | ZS:169425.2 | 1 | 281 |
| 101 | 701339683 | ZS:169425.2 | 1 | 245 |
| 103 | 700483806 | ZS:169425.2 | 3 | 279 |
| 104 | 700933808 | ZS:169425.2 | 1 | 247 |
| 105 | 700635387 | ZS:169425.2 | 1 | 248 |
| 106 | 701186639 | ZS:169425.2 | 1 | 254 |
| 107 | 700067896 | ZS:169425.2 | 1 | 201 |
| 108 | 701654036 | ZS:169425.2 | 2 | 321 |
| 109 | 701616311 | ZS:169425.2 | 7 | 292 |
| 110 | 700123547 | ZS:169425.2 | 5 | 232 |
| 111 | 701727660 | ZS:169425.2 | 4 | 270 |
| 112 | 5996159 | LG:2029581.1 | 2 | 135 |
| 113 | 3786708 | LG:2029581.1 | 1 | 188 |
| 114 | 4438637 | LG:2029040.1 | 1 | 186 |
| 115 | 3020423 | LG:2029040.1 | 1 | 272 |
| 116 | 4122257 | LG:2029040.1 | 1 | 89 |
| 117 | 5479570 | LG:1912304.1 | 1 | 256 |
| 118 | 5479760 | LG:1912304.1 | 1 | 138 |
| 119 | 5479960 | LG:1912304.1 | 1 | 173 |
| 120 | 5480114 | LG:1912304.1 | 1 | 218 |
| 121 | 5473892 | LG:1912304.1 | 1 | 192 |
| 122 | 5474141 | LG:1912304.1 | 1 | 226 |
| 123 | 5475667 | LG:1912304.1 | 1 | 206 |
| 124 | 5475060 | LG:1912304.1 | 1 | 269 |
| 125 | 5481168 | LG:1912304.1 | 1 | 177 |
| 126 | 5480121 | LG:1912304.1 | 1 | 283 |
| 127 | 5477881 | LG:1912304.1 | 1 | 212 |
| 128 | 5476185 | LG:1912304.1 | 1 | 256 |
| 129 | 5474878 | LG:1912304.1 | 1 | 258 |
| 130 | 5476523 | LG:1912304.1 | 1 | 209 |
| 131 | 6597357 | LG:2029581.4 | 1 | 362 |
| 132 | 6597429 | LG:2029581.4 | 1 | 355 |
| 133 | 3787304 | LG:2029581.4 | 58 | 251 |
| 134 | 6597257 | LG:2029581.4 | 1 | 223 |
| 135 | 6118845 | LG:2029581.4 | 1 | 402 |
| 136 | 6298190 | LG:1916065.1 | 7 | 265 |
| 137 | 6138222 | LG:1916065.1 | 2 | 295 |
| 138 | 1849578 | IB:1907955.2 | 1 | 83 |
| 139 | 661163 | IB:1907955.2 | 1 | 262 |
| 140 | 2101509 | IB:1907955.2 | 1 | 165 |
| 141 | 2923187 | IB:1907955.3 | 1 | 275 |
| 142 | 2052778 | IB:1907955.3 | 1 | 274 |
| 143 | 2347967 | IB:1907955.3 | 1 | 83 |
| 144 | 892747 | IB:1907955.3 | 1 | 209 |
| 145 | 154668 | IB:2096997.5 | 5 | 277 |
| 146 | 2473237 | IB:2096997.5 | 1 | 226 |
| 147 | 2173121 | IB:2096997.5 | 1 | 227 |
| 148 | 2048670 | IB:2096997.5 | 1 | 261 |
| 149 | 1289023 | IB:2096997.5 | 1 | 235 |
| 150 | 1997754 | IB:2096997.5 | 1 | 229 |
| 151 | 55605 | IB:2096997.5 | 1 | 191 |
| 152 | 2293016 | IB:2096997.5 | 13 | 229 |
| 153 | 1888743 | IB:2096997.5 | 1 | 255 |
| 154 | 1700085 | IB:2096997.5 | 1 | 198 |
| 155 | 2246684 | IB:2096997.5 | 1 | 254 |
| 156 | 1243521 | IB:2096997.5 | 1 | 231 |
| 157 | 1682120 | IB:2096997.5 | 1 | 196 |
| 158 | 519530 | IB:2096997.5 | 1 | 244 |
| 159 | 674959 | IB:2096997.5 | 1 | 258 |
| 160 | 2246604 | IB:2096997.5 | 1 | 265 |
| 161 | 1450183 | IB:2096997.5 | 1 | 231 |
| 162 | 676179 | IB:2096997.5 | 1 | 248 |
| 163 | 1603276 | IB:2096997.5 | 1 | 268 |
| 164 | 735309 | IB:2096997.5 | 1 | 237 |
| 165 | 1853346 | IB:2096997.5 | 1 | 265 |
| 166 | 1728347 | IB:2096997.5 | 1 | 180 |
| 167 | 2347508 | IB:2096997.5 | 1 | 242 |
| 168 | 770596 | IB:2096997.5 | 1 | 274 |
| 169 | 269698 | IB:2096997.5 | 2 | 357 |
| 170 | 2345639 | IB:2096997.5 | 1 | 103 |
| 171 | 2313282 | IB:2096997.5 | 1 | 211 |
| 172 | 536888 | IB:2096997.5 | 1 | 230 |
| 173 | 579434 | IB:2096997.5 | 1 | 266 |
| 174 | 809810 | IB:2096997.5 | 1 | 280 |
| 175 | 2903460 | IB:2096997.5 | 1 | 130 |
| 176 | 980041 | IB:2096997.5 | 1 | 307 |
| 177 | 158997 | IB:2096997.5 | 6 | 395 |
| 178 | 3468653 | IB:2096997.5 | 3 | 261 |
| 179 | 2497291 | IB:2096997.5 | 1 | 232 |
| 180 | 820929 | IB:2096997.5 | 1 | 263 |
| 181 | 767530 | IB:2096997.5 | 1 | 204 |
| 182 | 855022 | IB:2096997.5 | 13 | 239 |
| 183 | 32873 | IB:2096997.5 | 1 | 324 |
| 184 | 911244 | IB:2096997.5 | 1 | 103 |
| 185 | 1913132 | IB:2096997.5 | 1 | 104 |
| 186 | 772404 | IB:2096997.5 | 1 | 172 |
| 187 | 3115741 | IB:2096997.5 | 1 | 238 |
| 188 | 1750586 | IB:2096997.5 | 1 | 219 |
| 189 | 2259695 | IB:2096997.5 | 1 | 132 |
| 190 | 2913628 | IB:2096997.5 | 1 | 146 |
| 191 | 270440 | IB:2096997.5 | 1 | 185 |
| 192 | 2510064 | IB:2096997.5 | 1 | 288 |
| 193 | 1611520 | IB:2096997.5 | 1 | 129 |
| 194 | 30512 | IB:2096997.5 | 1 | 208 |
| 195 | 1557831 | IB:2096997.5 | 1 | 182 |
| 196 | 481021 | IB:2096997.5 | 2 | 166 |
| 197 | 2116071 | IB:2096997.5 | 1 | 263 |
| 198 | 989350 | IB:2096997.5 | 1 | 274 |
| 199 | 1806228 | IB:2096997.5 | 1 | 280 |
| 200 | 882564 | IB:2096997.5 | 1 | 216 |
| 201 | 2379423 | IB:2096997.5 | 1 | 229 |
| 202 | 994052 | IB:2096997.5 | 1 | 219 |
| 203 | 880888 | IB:2096997.5 | 1 | 244 |
| 204 | 764170 | IB:2096997.5 | 1 | 294 |
| 205 | 1564580 | IB:2096997.5 | 1 | 206 |
| 206 | 2170220 | IB:2096997.5 | 1 | 236 |
| 207 | 1898981 | IB:2096997.5 | 1 | 254 |
| 208 | 2262145 | IB:2096997.5 | 1 | 217 |
| 209 | 1290739 | IB:2096997.5 | 1 | 135 |
| 210 | 692787 | IB:2096997.5 | 1 | 140 |
| 211 | 1650650 | IB:2096997.5 | 1 | 190 |
| 212 | 56716 | IB:2096997.5 | 24 | 245 |
| 213 | 1856963 | IB:2096997.5 | 1 | 254 |
| 214 | 3028959 | IB:2096997.5 | 1 | 329 |
| 215 | 1580413 | IB:2096997.5 | 1 | 206 |
| 216 | 508309 | IB:2096997.5 | 1 | 204 |
| 217 | 2695558 | IB:2096997.5 | 1 | 284 |
| 218 | 1661051 | IB:2096997.5 | 1 | 232 |
| 219 | 693014 | IB:2096997.5 | 1 | 283 |
| 220 | 6413730 | LG:1899061.4 | 1 | 246 |
| 221 | 3927591 | IB:1907955.4 | 1 | 190 |
| 222 | 219065 | IB:1907955.4 | 1 | 204 |
| 223 | 217054 | IB:1907955.4 | 1 | 188 |
| 224 | 215070 | IB:1907955.4 | 1 | 243 |
| 225 | 219390 | IB:1907955.4 | 1 | 224 |
| 226 | 218904 | IB:1907955.4 | 1 | 231 |
| 227 | 77197 | IB:1907955.4 | 1 | 204 |
| 228 | 3928041 | IB:1907955.4 | 4 | 143 |
| 229 | 700532674 | ZS:162045.1 | 5 | 271 |
| 230 | 700520955 | ZS:162045.1 | 1 | 263 |
| 231 | 700785531 | ZS:162045.1 | 3 | 245 |
| 232 | 700525644 | ZS:162045.1 | 12 | 257 |
| 233 | 700524023 | ZS:162045.1 | 1 | 230 |
| 234 | 6112575 | LG:1899061.5 | 1 | 263 |
| 235 | 6385123 | LG:1899061.5 | 1 | 272 |
| 236 | 6424123 | LG:1899061.5 | 1 | 234 |
| 237 | 6396655 | LG:1899061.5 | 1 | 250 |
| 238 | 6178515 | LG:1899061.5 | 1 | 282 |
| 239 | 4440191 | LG:1899061.5 | 3 | 289 |
| 240 | 6386840 | LG:1899061.5 | 1 | 276 |
| 241 | 3438090 | IB:2097630.1 | 1 | 236 |
| 242 | 2103752 | IB:2025662.4 | 1 | 247 |
| 243 | 2134070 | IB:2098512.2 | 1 | 280 |
| 244 | 487437 | IB:2098512.2 | 1 | 239 |
| 245 | 2215250 | IB:2098512.2 | 1 | 187 |
| 246 | 345407 | IB:2098512.2 | 1 | 197 |
| 247 | 3244082 | IB:2098512.2 | 1 | 223 |
| 248 | 2827143 | IB:2095471.5 | 1 | 245 |
| 249 | 2821267 | IB:2095471.5 | 1 | 223 |
| 250 | 1742936 | IB:2098512.3 | 1 | 315 |
| 251 | 3335240 | IB:2098512.3 | 1 | 185 |
| 252 | 2747156 | IB:2098512.3 | 1 | 226 |
| 253 | 1355979 | IB:2098512.3 | 1 | 250 |
| 254 | 3187872 | IB:2098512.3 | 1 | 334 |
| 255 | 1369013 | IB:2098512.3 | 1 | 278 |
| 256 | 3565618 | IB:2098512.3 | 1 | 185 |
| 257 | 2953422 | IB:2098512.3 | 1 | 281 |
| 258 | 2331423 | IB:2098512.3 | 1 | 106 |
| 259 | 2837656 | IB:2098512.3 | 1 | 278 |
| 260 | 26403 | IB:2098512.3 | 1 | 222 |
| 261 | 2235510 | IB:2098512.3 | 1 | 262 |
| 262 | 1213194 | IB:2098512.3 | 1 | 210 |
| 263 | 2716655 | IB:2098512.3 | 1 | 257 |
| 264 | 1521103 | IB:2098512.3 | 1 | 209 |
| 265 | 1558940 | IB:2098512.3 | 1 | 218 |
| 266 | 1281986 | IB:2098512.3 | 1 | 245 |
| 267 | 2936440 | IB:2098512.3 | 1 | 249 |
| 268 | 2360924 | IB:2098512.3 | 1 | 223 |
| 269 | 721037 | IB:2098512.3 | 1 | 236 |
| 270 | 1282145 | IB:2098512.3 | 1 | 250 |
| 271 | 2045951 | IB:2098512.3 | 1 | 111 |
| 272 | 1606444 | IB:2098512.3 | 1 | 217 |
| 273 | 2285437 | IB:2098512.3 | 1 | 238 |
| 274 | 1512742 | IB:2095471.6 | 1 | 254 |
| 275 | 974641 | IB:2095471.6 | 1 | 189 |
| 276 | 1981621 | IB:2095471.6 | 1 | 268 |
| 277 | 547710 | IB:2095471.6 | 1 | 235 |
| 278 | 3452386 | IB:2095471.6 | 1 | 224 |
| 279 | 1541686 | IB:2095471.6 | 1 | 196 |
| 280 | 2508157 | IB:2095471.6 | 1 | 234 |
| 281 | 1336918 | IB:2095471.6 | 1 | 212 |
| 282 | 1524878 | IB:2095471.6 | 1 | 186 |
| 283 | 1003042 | IB:2095471.6 | 1 | 249 |
| 284 | 161598 | IB:2095471.6 | 1 | 388 |
| 285 | 28378 | IB:2095471.6 | 1 | 230 |
| 286 | 2231816 | IB:2095471.6 | 1 | 246 |
| 287 | 2638052 | IB:2095471.6 | 1 | 266 |
| 288 | 2284174 | IB:2095471.6 | 1 | 241 |
| 289 | 64295 | IB:2095471.6 | 1 | 135 |
| 290 | 672180 | IB:2095471.6 | 1 | 227 |
| 291 | 1683714 | IB:2095471.6 | 1 | 199 |
| 292 | 988257 | IB:2095471.6 | 1 | 296 |
| 293 | 178582 | IB:2095471.6 | 1 | 204 |
| 294 | 957959 | IB:2095471.6 | 1 | 104 |
| 295 | 805000 | IB:2095471.6 | 1 | 237 |
| 296 | 2963787 | IB:2095471.6 | 1 | 295 |
| 297 | 3255588 | IB:2095471.6 | 1 | 250 |
| 298 | 268277 | IB:2095471.6 | 1 | 407 |
| 299 | 132527 | IB:2095471.6 | 1 | 226 |
| 300 | 2351574 | IB:2095471.6 | 1 | 195 |
| 301 | 1427071 | IB:2095471.6 | 1 | 240 |
| 302 | 1302726 | IB:2095471.6 | 1 | 227 |
| 303 | 3014326 | IB:2095471.6 | 1 | 288 |
| 304 | 2793994 | IB:2095471.6 | 1 | 288 |
| 305 | 3361302 | IB:2095471.6 | 1 | 91 |
| 306 | 2228136 | IB:2095471.6 | 55 | 230 |
| 307 | 3439101 | IB:2095471.6 | 1 | 248 |
| 308 | 1284989 | IB:2095471.6 | 1 | 273 |
| 309 | 101709 | IB:2095471.6 | 1 | 172 |
| 310 | 2572961 | IB:2095471.6 | 1 | 252 |
| 311 | 1386853 | IB:2095471.6 | 1 | 268 |
| 312 | 968283 | IB:2095471.6 | 1 | 272 |
| 313 | 2434871 | IB:2095471.6 | 1 | 181 |
| 314 | 529702 | IB:2095471.6 | 1 | 247 |
| 315 | 691852 | IB:2095471.6 | 1 | 273 |
| 316 | 945009 | IB:2095471.6 | 1 | 243 |
| 317 | 38327 | IB:2095471.6 | 1 | 219 |
| 318 | 2662208 | IB:2095471.6 | 1 | 75 |
| 319 | 1236346 | IB:2095471.6 | 1 | 242 |
| 320 | 1671467 | IB:2095471.6 | 1 | 242 |
| 321 | 1450164 | IB:2095471.6 | 1 | 240 |
| 322 | 1661168 | IB:2095471.6 | 1 | 203 |
| 323 | 2675413 | IB:2095471.6 | 1 | 256 |
| 324 | 2071881 | IB:2095471.6 | 1 | 247 |
| 325 | 41595 | IB:2095471.6 | 1 | 289 |
| 326 | 3567727 | IB:2095471.6 | 1 | 167 |
| 327 | 438267 | IB:2095471.6 | 1 | 226 |
| 328 | 1670227 | IB:2095471.6 | 1 | 107 |
| 329 | 2733423 | IB:2095471.6 | 1 | 244 |
| 330 | 237 | IB:2095471.6 | 1 | 318 |
| 331 | 523746 | IB:2095471.6 | 1 | 189 |
| 332 | 1631414 | IB:2095471.6 | 1 | 206 |
| 333 | 3327940 | IB:2095471.6 | 1 | 263 |
| 334 | 436786 | IB:2095471.6 | 1 | 230 |
| 335 | 3036523 | IB:2095471.6 | 1 | 284 |
| 336 | 1724062 | IB:2095471.6 | 1 | 201 |
| 337 | 1773452 | IB:2095471.6 | 1 | 268 |
| 338 | 188 | IB:2095471.6 | 1 | 289 |
| 339 | 2728235 | IB:2095471.6 | 1 | 241 |
| 340 | 2657709 | IB:2095471.6 | 1 | 231 |
| 341 | 66598 | IB:2095471.6 | 1 | 161 |
| 342 | 2434736 | IB:2095471.6 | 1 | 189 |
| 343 | 2050370 | IB:2095471.6 | 1 | 259 |
| 344 | 858026 | IB:2095471.6 | 1 | 264 |
| 345 | 2922053 | IB:2095471.6 | 1 | 276 |
| 346 | 1486724 | IB:2095471.6 | 1 | 169 |
| 347 | 2319924 | IB:2095471.6 | 1 | 242 |
| 348 | 2584108 | IB:2095471.6 | 1 | 282 |
| 349 | 3318249 | IB:2095471.6 | 7 | 267 |
| 350 | 1418795 | IB:2095471.6 | 1 | 226 |
| 351 | 1490935 | IB:2095471.6 | 1 | 196 |
| 352 | 1972806 | IB:2095471.6 | 1 | 274 |
| 353 | 857740 | IB:2095471.6 | 1 | 211 |
| 354 | 777632 | IB:2095471.6 | 1 | 242 |
| 355 | 1443789 | IB:2095471.6 | 1 | 262 |
| 356 | 3360182 | IB:2095471.6 | 2 | 259 |
| 357 | 1490047 | IB:2095471.6 | 1 | 292 |
| 358 | 168121 | IB:2095471.6 | 1 | 340 |
| 359 | 1753578 | IB:2095471.6 | 1 | 187 |
| 360 | 1705762 | IB:2095471.6 | 1 | 243 |
| 361 | 947412 | IB:2095471.6 | 1 | 251 |
| 362 | 2769452 | IB:2095471.6 | 1 | 240 |
| 363 | 59936 | IB:2095471.6 | 1 | 218 |
| 364 | 975960 | IB:2095471.6 | 1 | 117 |
| 365 | 436218 | IB:2095471.6 | 1 | 200 |
| 366 | 2912496 | IB:2095471.6 | 1 | 277 |
| 367 | 1833183 | IB:2095471.6 | 1 | 232 |
| 368 | 3323085 | IB:2095471.6 | 1 | 255 |
| 369 | 2595534 | IB:2095471.6 | 1 | 253 |
| 370 | 158522 | IB:2095471.6 | 15 | 403 |
| 371 | 2739831 | IB:2095471.6 | 1 | 251 |
| 372 | 702877 | IB:2095471.6 | 1 | 173 |
| 373 | 2779245 | IB:2095471.6 | 1 | 259 |
| 374 | 3687255 | IB:2095471.6 | 2 | 275 |
| 375 | 1393468 | IB:2095471.6 | 1 | 257 |
| 376 | 870700 | IB:2095471.6 | 1 | 142 |
| 377 | 1629121 | IB:2095471.6 | 1 | 187 |
| 378 | 2616289 | IB:2095471.6 | 1 | 240 |
| 379 | 1225832 | IB:2095471.6 | 1 | 241 |
| 380 | 36001 | IB:2095471.6 | 1 | 121 |
| 381 | 541261 | IB:2095471.6 | 1 | 176 |
| 382 | 986764 | IB:2095471.6 | 1 | 161 |
| 383 | 3137305 | IB:2095471.6 | 1 | 277 |
| 384 | 1332445 | IB:2095471.6 | 1 | 257 |
| 385 | 1233856 | IB:2095471.6 | 1 | 146 |
| 386 | 2232952 | IB:2095471.6 | 1 | 168 |
| 387 | 838885 | IB:2095471.6 | 1 | 73 |
| 388 | 830015 | IB:2095471.6 | 1 | 279 |
| 389 | 968185 | IB:2095471.6 | 1 | 230 |
| 390 | 2266925 | IB:2095471.6 | 1 | 251 |
| 391 | 2232714 | IB:2095471.6 | 1 | 241 |
| 392 | 1618829 | IB:2095471.6 | 3 | 194 |
| 393 | 862083 | IB:2095471.6 | 1 | 255 |
| 394 | 567832 | IB:2095471.6 | 5 | 255 |
| 395 | 1737115 | IB:2095471.6 | 1 | 226 |
| 396 | 1565719 | IB:2095471.6 | 1 | 199 |
| 397 | 3319527 | IB:2095471.6 | 2 | 273 |
| 398 | 1860508 | IB:2095471.6 | 1 | 253 |
| 399 | 1674224 | IB:2095471.6 | 1 | 207 |
| 400 | 909282 | IB:2095471.6 | 1 | 298 |
| 401 | 2649949 | IB:2095471.6 | 1 | 264 |
| 402 | 2938018 | IB:2095471.6 | 1 | 265 |
| 403 | 2218628 | IB:2095471.6 | 1 | 261 |
| 404 | 2706495 | IB:2095471.6 | 1 | 293 |
| 405 | 105473 | IB:2095471.6 | 1 | 249 |
| 406 | 791482 | IB:2095471.6 | 1 | 219 |
| 407 | 3038523 | IB:2095471.6 | 1 | 262 |
| 408 | 754354 | IB:2095471.6 | 1 | 234 |
| 409 | 2588364 | IB:2095471.6 | 1 | 266 |
| 410 | 2467836 | IB:2095471.6 | 1 | 229 |
| 411 | 1807159 | IB:2095471.6 | 1 | 275 |
| 412 | 483414 | IB:2095471.6 | 1 | 224 |
| 413 | 1878392 | IB:2095471.6 | 1 | 270 |
| 414 | 3122394 | IB:2095471.6 | 1 | 284 |
| 415 | 1632586 | IB:2095471.6 | 1 | 203 |
| 416 | 1754034 | IB:2095471.6 | 1 | 255 |
| 417 | 2615938 | IB:2095471.6 | 1 | 240 |
| 418 | 776401 | IB:2095471.6 | 1 | 237 |
| 419 | 3253386 | IB:2095471.6 | 1 | 215 |
| 420 | 34572 | IB:2041967.1 | 178 | 299 |
| 421 | 5613187 | LG:2022251.2 | 1 | 202 |
| 422 | 198957 | IB:2017588.1 | 1 | 182 |
| 423 | 5613859 | LG:1915067.1 | 1 | 168 |
| 424 | 2241085 | IB:186634.1 | 1 | 239 |
| 425 | 6304773 | LG:2019095.3 | 1 | 482 |
| 426 | 5949169 | LG:2019095.3 | 1 | 131 |
| 427 | 5876719 | LG:2019095.3 | 1 | 297 |
| 428 | 6507116 | LG:2019095.3 | 1 | 164 |
| 429 | 6345009 | LG:2019095.3 | 1 | 296 |
| 430 | 6345041 | LG:2019095.3 | 3 | 294 |
| 431 | 6120407 | LG:2019095.3 | 1 | 554 |
| 432 | 6265395 | LG:2019095.3 | 1 | 520 |
| 433 | 2781758 | IB:2025662.9 | 1 | 243 |
| 434 | 1701981 | IB:2025662.9 | 1 | 194 |
| 435 | 759186 | IB:2025662.9 | 1 | 315 |
| 436 | 5657662 | LG:2002932.6 | 1 | 230 |
| 437 | 3787443 | LG:2002932.6 | 1 | 287 |
| 438 | 3788702 | LG:2002932.6 | 1 | 291 |
| 439 | 4668706 | LG:2002932.6 | 1 | 284 |
| 440 | 6056729 | LG:2002932.6 | 1 | 533 |
| 441 | 3762849 | LG:2002932.6 | 1 | 199 |
| 442 | 6558417 | LG:2002932.6 | 1 | 103 |
| 443 | 6559956 | LG:2002932.6 | 1 | 562 |
| 444 | 6210954 | LG:2002932.6 | 1 | 302 |
| 445 | 5808476 | LG:2002932.6 | 1 | 70 |
| 446 | 6555152 | LG:2002932.6 | 1 | 432 |
| 447 | 5888375 | LG:1994534.2 | 1 | 243 |
| 448 | 5880566 | LG:1994534.2 | 2 | 257 |
| 449 | 5884946 | LG:1994534.2 | 1 | 280 |
| 450 | 5889095 | LG:1994534.2 | 1 | 234 |
| 451 | 5880061 | LG:1994534.2 | 1 | 239 |
| 452 | 5890510 | LG:1994534.2 | 1 | 251 |
| 453 | 5885109 | LG:1994534.2 | 1 | 187 |
| 454 | 6129550 | LG:2002932.1 | 1 | 533 |
| 455 | 6127983 | LG:2002932.1 | 1 | 476 |
| 456 | 6111849 | LG:1994534.3 | 1 | 271 |
| 457 | 5695240 | LG:1994534.5 | 1 | 118 |
| 458 | 5815925 | LG:1994534.5 | 1 | 260 |
| 459 | 6138342 | LG:1994534.5 | 1 | 280 |
| 460 | 6522432 | LG:1994534.5 | 1 | 491 |
| 461 | 6310665 | LG:1994534.5 | 1 | 532 |
| 462 | 6129270 | LG:1994534.5 | 1 | 394 |
| 463 | 6408546 | LG:1994534.5 | 1 | 593 |
| 464 | 6305061 | LG:1994534.5 | 1 | 534 |
| 465 | 6588841 | LG:1994534.5 | 16 | 155 |
| 466 | 6552781 | LG:1994534.5 | 1 | 550 |
| 467 | 6482476 | LG:1994534.5 | 1 | 600 |
| 468 | 6343922 | LG:1994534.5 | 1 | 176 |
| 469 | 5919979 | LG:1994534.5 | 1 | 292 |
| 470 | 3783111 | LG:1994534.5 | 1 | 306 |
| 471 | 4518248 | LG:1994534.5 | 1 | 243 |
| 472 | 2492677 | IB:2097928.2 | 1 | 144 |
| 473 | 3357454 | IB:2073270.1 | 1 | 279 |
| 474 | 6126885 | LG:2002932.3 | 1 | 535 |
| 475 | 188581 | IB:2097928.3 | 1 | 142 |
| 476 | 2135063 | IB:2097928.3 | 1 | 277 |
| 477 | 1331867 | IB:2097928.4 | 1 | 251 |
| 478 | 3960113 | IB:2097928.4 | 1 | 270 |
| 479 | 1644841 | IB:2097928.4 | 1 | 208 |
| 480 | 2081894 | IB:2097928.4 | 1 | 255 |
| 481 | 3069069 | IB:2097928.4 | 1 | 295 |
| 482 | 4323184 | IB:2097928.4 | 1 | 258 |
| 483 | 2539031 | IB:2097928.4 | 1 | 228 |
| 484 | 742282 | IB:2097928.4 | 1 | 213 |
| 485 | 2028355 | IB:2097928.4 | 1 | 76 |
| 486 | 159425 | IB:2097928.4 | 1 | 203 |
| 487 | 671593 | IB:2097928.4 | 1 | 259 |
| 488 | 2604050 | IB:2097928.4 | 1 | 147 |
| 489 | 3685138 | IB:2097928.4 | 1 | 190 |
| 490 | 3957184 | IB:2097928.4 | 1 | 278 |
| 491 | 2908269 | IB:2097928.4 | 1 | 296 |
| 492 | 942890 | IB:2097928.4 | 1 | 289 |
| 493 | 1332741 | IB:2097928.4 | 1 | 248 |
| 494 | 1316096 | IB:2097928.4 | 1 | 227 |
| 495 | 289538 | IB:2097928.4 | 1 | 187 |
| 496 | 679064 | IB:2097928.4 | 1 | 245 |
| 497 | 2439313 | IB:2097928.4 | 1 | 260 |
| 498 | 909094 | IB:2097928.4 | 1 | 300 |
| 499 | 2670325 | IB:2097928.4 | 1 | 247 |
| 500 | 2443220 | IB:2097928.4 | 1 | 123 |
| 501 | 2149587 | IB:2097928.4 | 1 | 267 |
| 502 | 291706 | IB:2097928.4 | 1 | 80 |
| 503 | 2665513 | IB:2097928.4 | 1 | 238 |
| 504 | 2953235 | IB:2097928.4 | 1 | 283 |
| 505 | 890040 | IB:2097928.4 | 1 | 198 |
| 506 | 1609748 | IB:2097928.4 | 1 | 169 |
| 507 | 2293179 | IB:2097928.4 | 1 | 233 |
| 508 | 2766633 | IB:2097928.4 | 1 | 245 |
| 509 | 2736649 | IB:2097928.4 | 1 | 270 |
| 510 | 2304562 | IB:2097928.4 | 1 | 104 |
| 511 | 2763026 | IB:2097928.4 | 1 | 253 |
| 512 | 1377278 | IB:2097928.4 | 1 | 239 |
| 513 | 1998146 | IB:2097928.4 | 1 | 249 |
| 514 | 2790194 | IB:2097928.4 | 1 | 302 |
| 515 | 2530965 | IB:2097928.4 | 1 | 234 |
| 516 | 2664895 | IB:2097928.4 | 1 | 245 |
| 517 | 1836569 | IB:2097928.4 | 37 | 103 |
| 518 | 1281254 | IB:2097928.4 | 1 | 228 |
| 519 | 2658750 | IB:2097928.4 | 1 | 229 |
| 520 | 1468402 | IB:2097928.4 | 1 | 185 |
| 521 | 94971 | IB:2097928.4 | 39 | 226 |
| 522 | 3202791 | IB:2097928.4 | 1 | 269 |
| 523 | 1623750 | IB:2097928.4 | 1 | 170 |
| 524 | 2269607 | IB:2097928.4 | 1 | 249 |
| 525 | 555662 | IB:2097928.4 | 2 | 70 |
| 526 | 3273425 | IB:2097928.4 | 1 | 237 |
| 527 | 488117 | IB:2097928.4 | 9 | 239 |
| 528 | 955963 | IB:2097928.4 | 1 | 236 |
| 529 | 837468 | IB:2097928.4 | 1 | 257 |
| 530 | 2668611 | IB:2097928.4 | 1 | 243 |
| 531 | 641056 | IB:2097928.4 | 2 | 263 |
| 532 | 1624341 | IB:2097928.4 | 1 | 205 |
| 533 | 2152009 | IB:2097928.4 | 1 | 97 |
| 534 | 672412 | IB:2097928.4 | 2 | 231 |
| 535 | 68947 | IB:2097928.4 | 1 | 204 |
| 536 | 1312213 | IB:2097928.4 | 1 | 76 |
| 537 | 738387 | IB:2097928.4 | 1 | 227 |
| 538 | 1869727 | IB:2097928.4 | 1 | 277 |
| 539 | 2834526 | IB:2097928.4 | 1 | 271 |
| 540 | 1547894 | IB:2097928.4 | 1 | 87 |
| 541 | 3566219 | IB:2097928.4 | 1 | 302 |
| 542 | 114170 | IB:2097928.4 | 1 | 208 |
| 543 | 2264814 | IB:2097928.4 | 1 | 272 |
| 544 | 219339 | IB:2097928.4 | 1 | 67 |
| 545 | 733063 | IB:2097928.4 | 1 | 274 |
| 546 | 3118194 | IB:2097928.4 | 1 | 278 |
| 547 | 2661257 | IB:2097928.4 | 1 | 278 |
| 548 | 2990381 | IB:2097928.4 | 2 | 260 |
| 549 | 548160 | IB:2097928.4 | 1 | 239 |
| 550 | 1370295 | IB:2097928.4 | 1 | 223 |
| 551 | 1887457 | IB:2097928.4 | 1 | 259 |
| 552 | 2210818 | IB:2097928.4 | 1 | 261 |
| 553 | 3176964 | IB:2097928.4 | 1 | 175 |
| 554 | 4209737 | IB:2097928.4 | 1 | 265 |
| 555 | 687475 | IB:2097928.4 | 1 | 178 |
| 556 | 3461089 | IB:2097928.4 | 1 | 234 |
| 557 | 158748 | IB:2097928.4 | 1 | 190 |
| 558 | 583799 | IB:2097928.4 | 1 | 76 |
| 559 | 2784522 | IB:2097928.4 | 1 | 250 |
| 560 | 2441763 | IB:2097928.4 | 1 | 217 |
| 561 | 741986 | IB:2097928.4 | 1 | 263 |
| 562 | 3336778 | IB:2097928.4 | 1 | 243 |
| 563 | 2631182 | IB:2097928.4 | 1 | 242 |
| 564 | 630531 | IB:2097928.4 | 1 | 272 |
| 565 | 2371227 | IB:2097928.4 | 1 | 236 |
| 566 | 1001180 | IB:2097928.4 | 1 | 254 |
| 567 | 270390 | IB:2097928.4 | 1 | 346 |
| 568 | 46859 | IB:2097928.4 | 1 | 258 |
| 569 | 630234 | IB:2097928.4 | 1 | 224 |
| 570 | 1991586 | IB:2097928.4 | 1 | 225 |
| 571 | 3925490 | IB:2097928.4 | 1 | 177 |
| 572 | 3401566 | IB:2097928.4 | 1 | 245 |
| 573 | 3338452 | IB:2097928.4 | 1 | 260 |
| 574 | 1921936 | IB:2097928.4 | 1 | 261 |
| 575 | 2406413 | IB:2097928.4 | 1 | 230 |
| 576 | 3271032 | IB:2097928.4 | 1 | 245 |
| 577 | 579510 | IB:2097928.4 | 1 | 255 |
| 578 | 1999081 | IB:2097928.4 | 1 | 114 |
| 579 | 2118363 | IB:2097928.4 | 1 | 173 |
| 580 | 2650642 | IB:2097928.4 | 1 | 230 |
| 581 | 2766825 | IB:2097928.4 | 1 | 231 |
| 582 | 94970 | IB:2097928.4 | 1 | 229 |
| 583 | 3173908 | IB:2097928.4 | 2 | 175 |
| 584 | 837155 | IB:2097928.4 | 1 | 255 |
| 585 | 547537 | IB:2097928.4 | 1 | 251 |
| 586 | 672497 | IB:2097928.4 | 1 | 247 |
| 587 | 2720987 | IB:2097928.4 | 1 | 234 |
| 588 | 1003869 | IB:2097928.4 | 1 | 239 |
| 589 | 1259346 | IB:2097928.4 | 1 | 253 |
| 590 | 2474822 | IB:2097928.4 | 1 | 238 |
| 591 | 6120495 | LG:2002932.5 | 1 | 576 |
| 592 | 6030522 | LG:2002932.5 | 1 | 257 |
| 593 | 6127887 | LG:2002932.5 | 1 | 583 |
| 594 | 550609 | IB:1916065.1 | 1 | 271 |
| 595 | 2834338 | IB:1916065.1 | 1 | 269 |
| 596 | 1209024 | IB:1916065.1 | 1 | 221 |
| 597 | 2160735 | IB:1916065.1 | 1 | 268 |
| 598 | 1006278 | IB:1916065.1 | 1 | 254 |
| 599 | 2588088 | IB:1916065.1 | 1 | 244 |
| 600 | 2160580 | IB:1916065.1 | 1 | 253 |
| 601 | 6523403 | LG:2028962.1 | 1 | 604 |
| 602 | 5953606 | LG:2016091.1 | 1 | 237 |
| 603 | 6372764 | LG:2016091.1 | 1 | 267 |
| 604 | 6373435 | LG:2016091.1 | 1 | 108 |
| 605 | 6387476 | LG:2016091.1 | 1 | 248 |
| 606 | 6410750 | LG:2016091.1 | 1 | 279 |
| 607 | 6372052 | LG:2016091.1 | 1 | 278 |
| 608 | 6305576 | LG:2016091.1 | 14 | 550 |
| 609 | 6122907 | LG:2016091.1 | 1 | 550 |
| 610 | 6422660 | LG:2016091.1 | 1 | 518 |
| 611 | 6454929 | LG:2016091.1 | 1 | 563 |
| 612 | 6383359 | LG:2016091.1 | 1 | 271 |
| 613 | 6422668 | LG:2016091.1 | 1 | 535 |
| 614 | 6118137 | LG:2016091.1 | 1 | 433 |
| 615 | 6126413 | LG:2016091.1 | 6 | 348 |
| 616 | 6425148 | LG:2016091.1 | 1 | 462 |
| 617 | 1857027 | IB:1899061.1 | 1 | 237 |
| 618 | 700368142 | ZS:157650.3 | 1 | 270 |
| 619 | 700494046 | ZS:157650.3 | 3 | 94 |
| 620 | 700369876 | ZS:157650.3 | 14 | 183 |
| 621 | 700255036 | ZS:157650.3 | 7 | 136 |
| 622 | 700494879 | ZS:157650.3 | 3 | 277 |
| 623 | 467072 | IB:1899061.2 | 1 | 221 |
| 624 | 464308 | IB:1899061.2 | 1 | 199 |
| 625 | 2798569 | IB:1899061.3 | 1 | 95 |
| 626 | 2230026 | IB:1899061.3 | 1 | 218 |
| 627 | 700536442 | ZS:157650.5 | 1 | 284 |
| 628 | 701608605 | ZS:157650.5 | 1 | 178 |
| 629 | 700815950 | ZS:157650.5 | 1 | 227 |
| 630 | 700918115 | ZS:157650.5 | 1 | 262 |
| 631 | 701257014 | ZS:157650.5 | 9 | 216 |
| 632 | 700461506 | ZS:157650.5 | 1 | 180 |
| 634 | 701346456 | ZS:157650.5 | 1 | 181 |
| 635 | 700123372 | ZS:157650.5 | 1 | 241 |
| 636 | 700774145 | ZS:157650.5 | 42 | 235 |
| 637 | 701608169 | ZS:157650.5 | 16 | 253 |
| 638 | 700322834 | ZS:157650.5 | 1 | 235 |
| 639 | 701195154 | ZS:157650.5 | 1 | 226 |
| 640 | 701509245 | ZS:157650.5 | 1 | 246 |
| 641 | 700067453 | ZS:157650.5 | 2 | 277 |
| 642 | 700478133 | ZS:157650.5 | 1 | 282 |
| 643 | 700372405 | ZS:157650.5 | 1 | 161 |
| 644 | 700175226 | ZS:157650.5 | 1 | 244 |
| 645 | 700060734 | ZS:157650.5 | 1 | 264 |
| 646 | 700419934 | ZS:157650.5 | 5 | 284 |
| 647 | 701444306 | ZS:157650.5 | 1 | 206 |
| 648 | 701720942 | ZS:157650.5 | 1 | 285 |
| 649 | 700420845 | ZS:157650.5 | 3 | 217 |
| 650 | 700365506 | ZS:157650.5 | 1 | 266 |
| 651 | 701474948 | ZS:157650.5 | 3 | 277 |
| 652 | 700323207 | ZS:157650.5 | 1 | 226 |
| 653 | 700067452 | ZS:157650.5 | 2 | 178 |
| 654 | 700376957 | ZS:157650.5 | 1 | 100 |
| 655 | 1307933 | IB:1899061.4 | 1 | 194 |
| 656 | 3974283 | IB:1899061.4 | 3 | 268 |
| 657 | 3422227 | IB:1899061.4 | 1 | 237 |
| 658 | 721822 | IB:1899061.4 | 1 | 233 |
| 659 | 1839034 | IB:1899061.4 | 1 | 283 |
| 660 | 3210659 | IB:1899061.4 | 1 | 309 |
| 661 | 2226674 | IB:1899061.4 | 1 | 226 |
| 662 | 1432972 | IB:1899061.4 | 1 | 219 |
| 663 | 2243725 | IB:1899061.4 | 1 | 190 |
| 664 | 2546759 | IB:1899061.4 | 1 | 235 |
| 665 | 259745 | IB:1899061.4 | 1 | 357 |
| 666 | 41970 | IB:1899061.4 | 1 | 260 |
| 667 | 1592324 | IB:1899061.4 | 1 | 192 |
| 668 | 3898065 | IB:1899061.4 | 1 | 233 |
| 669 | 2735908 | IB:1899061.4 | 1 | 246 |
| 670 | 2435175 | IB:1899061.4 | 1 | 241 |
| 671 | 2770076 | IB:1899061.4 | 1 | 252 |
| 672 | 1268496 | IB:1899061.4 | 1 | 237 |
| 673 | 523940 | IB:1899061.4 | 1 | 227 |
| 674 | 2365873 | IB:1899061.4 | 1 | 234 |
| 675 | 888182 | IB:1899061.4 | 1 | 243 |
| 676 | 2471553 | IB:1899061.4 | 1 | 230 |
| 677 | 2360352 | IB:1899061.4 | 173 | 270 |
| 678 | 2804594 | IB:1899061.4 | 1 | 294 |
| 679 | 2684315 | IB:1899061.4 | 1 | 237 |
| 680 | 3181620 | IB:1899061.4 | 1 | 305 |
| 681 | 3566603 | IB:1899061.4 | 2 | 228 |
| 682 | 3130394 | IB:1899061.4 | 1 | 271 |
| 683 | 735448 | IB:1899061.4 | 1 | 208 |
| 684 | 2149570 | IB:1899061.4 | 1 | 135 |
| 685 | 1257772 | IB:1899061.4 | 1 | 225 |
| 686 | 2728309 | IB:1899061.4 | 1 | 252 |
| 687 | 1468878 | IB:1899061.4 | 1 | 194 |
| 688 | 4349373 | IB:1899061.4 | 1 | 269 |
| 689 | 2095174 | IB:1899061.4 | 1 | 268 |
| 690 | 2588957 | IB:1899061.4 | 1 | 200 |
| 691 | 1991653 | IB:1899061.4 | 1 | 270 |
| 692 | 2257385 | IB:1899061.4 | 1 | 71 |
| 693 | 424822 | IB:1899061.4 | 1 | 222 |
| 694 | 2764187 | IB:1899061.4 | 1 | 234 |
| 695 | 440571 | IB:1899061.4 | 1 | 213 |
| 696 | 1451469 | IB:1899061.4 | 1 | 246 |
| 697 | 828745 | IB:1899061.4 | 1 | 284 |
| 698 | 1982096 | IB:1899061.4 | 1 | 221 |
| 699 | 2986448 | IB:1899061.4 | 1 | 281 |
| 700 | 3319382 | IB:1899061.4 | 2 | 261 |
| 701 | 2681484 | IB:1899061.4 | 5 | 249 |
| 702 | 2279834 | IB:1899061.4 | 1 | 217 |
| 703 | 3201139 | IB:1899061.4 | 1 | 271 |
| 704 | 2696415 | IB:1899061.4 | 1 | 188 |
| 705 | 79005 | IB:1899061.4 | 1 | 253 |
| 706 | 1307950 | IB:1899061.4 | 1 | 200 |
| 707 | 2322036 | IB:1899061.4 | 1 | 261 |
| 708 | 2777457 | IB:1899061.4 | 1 | 252 |
| 709 | 1801673 | IB:1899061.4 | 1 | 276 |
| 710 | 2232170 | IB:1899061.4 | 1 | 228 |
| 711 | 1311164 | IB:1899061.4 | 1 | 232 |
| 712 | 4346781 | IB:1899061.4 | 1 | 250 |
| 713 | 1271781 | IB:1899061.4 | 1 | 225 |
| 714 | 1451452 | IB:1899061.4 | 1 | 253 |
| 715 | 2503478 | IB:1899061.4 | 1 | 236 |
| 716 | 2494513 | IB:1899061.4 | 1 | 329 |
| 717 | 3325740 | IB:1899061.4 | 1 | 276 |
| 718 | 1505327 | IB:1899061.4 | 1 | 222 |
| 719 | 1613557 | IB:1899061.4 | 1 | 198 |
| 720 | 1254974 | IB:1899061.4 | 1 | 260 |
| 721 | 2452005 | IB:1899061.4 | 1 | 181 |
| 722 | 2612202 | IB:1899061.4 | 1 | 190 |
| 723 | 35154 | IB:1899061.4 | 1 | 224 |
| 724 | 3960285 | IB:1899061.4 | 1 | 261 |
| 725 | 985780 | IB:1899061.4 | 1 | 258 |
| 726 | 1839341 | IB:1899061.4 | 1 | 272 |
| 727 | 1982581 | IB:1899061.4 | 1 | 139 |
| 728 | 1931150 | IB:1899061.4 | 1 | 268 |
| 729 | 2452908 | IB:1899061.4 | 1 | 234 |
| 730 | 6523007 | LG:2021143.3 | 1 | 540 |
| 731 | 6523607 | LG:2021143.3 | 1 | 562 |
| 732 | 700434903 | ZS:162199.1 | 3 | 150 |
| 733 | 700934290 | ZS:162199.1 | 1 | 116 |
| 734 | 700418829 | ZS:162199.1 | 1 | 109 |
| 735 | 700249262 | ZS:162199.1 | 9 | 298 |
| 736 | 700418821 | ZS:162199.1 | 1 | 110 |
| 737 | 700821102 | ZS:162199.1 | 1 | 92 |
| 738 | 701580728 | ZS:162199.1 | 3 | 217 |
| 739 | 700435587 | ZS:162199.1 | 4 | 205 |
| 740 | 3328503 | IB:2095693.4 | 1 | 255 |
| 741 | 40700 | IB:2095693.6 | 1 | 162 |
| 742 | 5678683 | LG:2021143.7 | 1 | 242 |
| 743 | 6432644 | LG:2021143.7 | 1 | 430 |
| 744 | 6010062 | LG:2021143.7 | 1 | 179 |
| 745 | 6556049 | LG:2021143.7 | 1 | 501 |
| 746 | 6107822 | LG:2021143.7 | 2 | 269 |
| 747 | 6078778 | LG:2021143.7 | 1 | 291 |
| 748 | 6381042 | LG:2021143.7 | 1 | 230 |
| 749 | 6612661 | LG:2021143.7 | 1 | 526 |
| 750 | 6544335 | LG:2021143.7 | 1 | 566 |
| 751 | 6429658 | LG:2021143.7 | 1 | 437 |
| 752 | 5877503 | LG:2021143.7 | 1 | 287 |
| 753 | 6603752 | LG:2021143.7 | 1 | 543 |
| 754 | 6564202 | LG:2021143.7 | 1 | 416 |
| 755 | 6514218 | LG:2021143.7 | 1 | 496 |
| 756 | 4519243 | LG:2021143.7 | 1 | 240 |
| 757 | 4519149 | LG:2021143.7 | 1 | 278 |
| 758 | 6402167 | LG:2021143.7 | 1 | 279 |
| 759 | 3162188 | LG:2021143.7 | 2 | 253 |
| 760 | 6544193 | LG:2021143.7 | 1 | 465 |
| 761 | 5564022 | LG:2021143.7 | 1 | 249 |
| 762 | 3762964 | LG:2021143.7 | 4 | 168 |
| 763 | 85665 | IB:2095693.7 | 5 | 180 |
| 764 | 78988 | IB:2095693.7 | 1 | 212 |
| 765 | 72104 | IB:2095693.7 | 1 | 178 |
| 766 | 69718 | IB:2095693.7 | 3 | 178 |
| 767 | 649490 | IB:2095693.7 | 1 | 251 |
| 768 | 26609 | IB:2095693.7 | 1 | 229 |
| 769 | 41719 | IB:2095693.7 | 3 | 214 |
| 770 | 77013 | IB:2095693.7 | 1 | 218 |
| 771 | 1862465 | IB:2095693.7 | 1 | 114 |
| 772 | 3179407 | IB:2095693.7 | 1 | 326 |
| 773 | 68007 | IB:2095693.7 | 1 | 210 |
| 774 | 3079952 | IB:2095693.7 | 3 | 305 |
| 775 | 892 | IB:2095693.7 | 3 | 410 |
| 776 | 581245 | IB:2095693.7 | 1 | 281 |
| 777 | 1416 | IB:2095693.7 | 1 | 399 |
| 778 | 536352 | IB:2095693.7 | 1 | 119 |
| 779 | 4213912 | IB:2095693.7 | 1 | 213 |
| 781 | 2914553 | IB:2095693.7 | 8 | 183 |
| 782 | 578971 | IB:2095693.7 | 1 | 233 |
| 783 | 700768273 | ZS:158823.1 | 1 | 279 |
| 784 | 701242681 | ZS:158823.1 | 1 | 130 |
| 785 | 701246365 | ZS:158823.1 | 1 | 260 |
| 786 | 701188745 | ZS:158823.1 | 1 | 153 |
| 787 | 700496220 | ZS:158823.1 | 1 | 279 |
| 788 | 700370466 | ZS:158823.1 | 3 | 236 |
| 789 | 700315154 | ZS:158823.1 | 1 | 159 |
| 790 | 700497170 | ZS:158823.1 | 2 | 91 |
| 791 | 700770692 | ZS:158823.1 | 2 | 98 |
| 792 | 700290724 | ZS:158823.1 | 6 | 286 |
| 793 | 700367624 | ZS:158823.1 | 1 | 80 |
| 794 | 700066526 | ZS:158823.1 | 1 | 249 |
| 795 | 700061414 | ZS:158823.1 | 1 | 68 |
| 796 | 701482761 | ZS:158823.1 | 1 | 143 |
| 797 | 700541247 | ZS:158823.1 | 1 | 203 |
| 798 | 701193468 | ZS:158823.1 | 1 | 268 |
| 799 | 700606709 | ZS:158823.1 | 2 | 140 |
| 800 | 700420587 | ZS:158823.1 | 1 | 128 |
| 801 | 700593713 | ZS:158823.1 | 1 | 209 |
| 802 | 700928945 | ZS:158823.1 | 1 | 74 |
| 803 | 701189016 | ZS:158823.1 | 1 | 217 |
| 804 | 1986692 | IB:2093542.4 | 1 | 212 |
| 805 | 2270579 | IB:2093542.4 | 1 | 266 |
| 806 | 3274021 | IB:2093542.4 | 1 | 267 |
| 807 | 1347425 | IB:2093542.4 | 1 | 231 |
| 808 | 864155 | IB:2093542.4 | 1 | 251 |
| 809 | 1423768 | IB:2093542.4 | 1 | 221 |
| 810 | 2355551 | IB:2093542.4 | 1 | 238 |
| 811 | 3325114 | IB:2093542.4 | 1 | 287 |
| 812 | 1296794 | IB:2093542.4 | 1 | 246 |
| 813 | 1340754 | IB:2093542.4 | 1 | 265 |
| 814 | 2695839 | IB:2093542.4 | 1 | 295 |
| 815 | 1746423 | IB:2093542.4 | 1 | 250 |
| 816 | 1502674 | IB:2093542.4 | 1 | 283 |
| 817 | 2899137 | IB:2093542.4 | 1 | 289 |
| 818 | 1294153 | IB:2093542.4 | 1 | 254 |
| 819 | 3256826 | IB:2093542.4 | 1 | 248 |
| 820 | 3244929 | IB:2093542.4 | 1 | 256 |
| 821 | 1739432 | IB:2093542.4 | 1 | 181 |
| 822 | 3116132 | IB:2093542.4 | 1 | 293 |
| 823 | 1386061 | IB:2093542.4 | 1 | 164 |
| 824 | 3337508 | IB:2093542.4 | 1 | 260 |
| 825 | 1905370 | IB:2022179.1 | 1 | 228 |
| 826 | 2050014 | IB:2104284.5 | 1 | 273 |
| 827 | 1972386 | IB:2104284.5 | 1 | 267 |
| 828 | 463172 | IB:2104284.5 | 1 | 227 |
| 829 | 1490003 | IB:2104284.5 | 1 | 266 |
| 830 | 239023 | IB:2104284.5 | 1 | 214 |
| 831 | 2096680 | IB:2104284.5 | 1 | 242 |
| 832 | 2910217 | IB:2104284.5 | 1 | 266 |
| 833 | 1698871 | IB:2104284.5 | 1 | 234 |
| 834 | 2472307 | IB:2104284.5 | 1 | 239 |
| 835 | 1689376 | IB:2104284.5 | 1 | 203 |
| 836 | 995171 | IB:2104284.5 | 1 | 229 |
| 837 | 3323088 | IB:2104284.5 | 1 | 254 |
| 838 | 2758260 | IB:2104284.5 | 1 | 227 |
| 839 | 3448616 | IB:2104284.5 | 1 | 250 |
| 840 | 3292975 | IB:2104284.5 | 1 | 247 |
| 841 | 763788 | IB:2104284.5 | 1 | 70 |
| 842 | 2476089 | IB:2104284.5 | 1 | 234 |
| 843 | 3451002 | IB:2104284.5 | 1 | 125 |
| 844 | 1625423 | IB:2104284.5 | 1 | 203 |
| 845 | 2682429 | IB:2104284.5 | 2 | 269 |
| 846 | 491356 | IB:2104284.5 | 1 | 260 |
| 847 | 1811236 | IB:2104284.5 | 1 | 251 |
| 848 | 2598057 | IB:2104284.5 | 1 | 130 |
| 849 | 1984010 | IB:2104284.5 | 1 | 256 |
| 850 | 2830524 | IB:2104284.5 | 1 | 266 |
| 851 | 8248 | IB:2104284.5 | 4 | 290 |
| 852 | 321613 | IB:2104284.5 | 1 | 255 |
| 853 | 677686 | IB:2104284.5 | 1 | 245 |
| 854 | 2584176 | IB:2104284.5 | 1 | 277 |
| 855 | 638241 | IB:2104284.5 | 1 | 251 |
| 856 | 257820 | IB:2104284.5 | 1 | 193 |
| 857 | 506236 | IB:2104284.5 | 1 | 203 |
| 858 | 2645110 | IB:2104284.5 | 1 | 251 |
| 859 | 1957671 | IB:2104284.5 | 1 | 262 |
| 860 | 2682468 | IB:2104284.5 | 1 | 211 |
| 861 | 1302 | IB:2104284.5 | 1 | 303 |
| 862 | 3361311 | IB:2104284.5 | 2 | 248 |
| 863 | 1355750 | IB:2104284.5 | 1 | 265 |
| 864 | 2173973 | IB:2104284.5 | 1 | 238 |
| 865 | 57539 | IB:2104284.5 | 1 | 188 |
| 866 | 2613128 | IB:2104284.5 | 1 | 233 |
| 867 | 2782774 | IB:2104284.5 | 1 | 257 |
| 868 | 1703167 | IB:2022179.6 | 1 | 245 |
| 869 | 806627 | IB:2022179.6 | 1 | 210 |
| 870 | 1831627 | IB:2022179.6 | 1 | 262 |
| 871 | 381954 | IB:2022179.6 | 5 | 200 |
| 872 | 3245690 | IB:2022179.6 | 1 | 251 |
| 873 | 1593378 | IB:2022179.6 | 1 | 237 |
| 874 | 928044 | IB:2022179.6 | 1 | 172 |
| 875 | 1627474 | IB:2022179.6 | 1 | 212 |
| 876 | 2750679 | IB:2022179.6 | 1 | 278 |
| 877 | 691867 | IB:2093542.9 | 1 | 255 |
| 878 | 688682 | IB:2093542.9 | 1 | 243 |
| 879 | 5865786 | LG:2000458.1 | 1 | 267 |
| 880 | 6179644 | LG:1924906.1 | 1 | 316 |
| 881 | 1903494 | IB:2095467.1 | 1 | 237 |
| 882 | 434242 | IB:2101151.1 | 1 | 225 |
| 883 | 3139053 | IB:2101151.1 | 1 | 282 |
| 884 | 864652 | IB:2101151.1 | 10 | 189 |
| 885 | 631104 | IB:2101151.1 | 1 | 248 |
| 886 | 1729479 | IB:2101151.1 | 1 | 228 |
| 887 | 6423435 | LG:1997596.3 | 1 | 476 |
| 888 | 5712404 | LG:1997596.3 | 1 | 278 |
| 889 | 6100191 | LG:1997596.3 | 1 | 279 |
| 890 | 6096811 | LG:1997596.3 | 4 | 324 |
| 891 | 6411280 | LG:1997596.3 | 2 | 161 |
| 892 | 6514626 | LG:1997596.3 | 1 | 322 |
| 893 | 3787301 | LG:1997596.3 | 1 | 70 |
| 894 | 6565167 | LG:1997596.3 | 1 | 490 |
| 895 | 4667847 | LG:1997596.3 | 1 | 106 |
| 896 | 6411253 | LG:1997596.3 | 1 | 288 |
| 897 | 5842026 | LG:1997596.3 | 1 | 282 |
| 898 | 4518594 | LG:1997596.3 | 1 | 251 |
| 899 | 6541505 | LG:1997596.3 | 1 | 559 |
| 900 | 2597277 | IB:2022623.5 | 1 | 246 |
| 901 | 923474 | IB:2022623.5 | 1 | 273 |
| 902 | 1475844 | IB:2022623.6 | 1 | 182 |
| 903 | 5618571 | LG:2001822.2 | 1 | 303 |
| 904 | 6246764 | LG:2001822.2 | 1 | 579 |
| 905 | 5700115 | LG:2001822.2 | 3 | 260 |
| 906 | 5951238 | LG:2001822.2 | 3 | 333 |
| 907 | 6390391 | LG:2001822.2 | 2 | 249 |
| 908 | 6390312 | LG:2001822.2 | 2 | 239 |
| 909 | 3567447 | LG:2001822.2 | 1 | 155 |
| 910 | 6247056 | LG:2001822.2 | 1 | 603 |
| 911 | 5615260 | LG:2001822.2 | 7 | 276 |
| 912 | 1786291 | IB:2022623.8 | 1 | 263 |
| 913 | 2779480 | IB:2022623.8 | 1 | 209 |
| 914 | 1315024 | IB:2022623.8 | 1 | 245 |
| 915 | 1786745 | IB:2022623.8 | 1 | 226 |
| 916 | 1258759 | IB:2022623.8 | 1 | 208 |
| 917 | 1547727 | IB:2022623.8 | 1 | 187 |
| 918 | 2100659 | IB:2022623.8 | 1 | 230 |
| 919 | 2839374 | IB:2022623.8 | 1 | 273 |
| 920 | 1634458 | IB:2022623.8 | 1 | 211 |
| 921 | 2702257 | IB:2022623.8 | 1 | 276 |
| 922 | 2356088 | IB:1081640.1 | 1 | 105 |
| 923 | 978769 | IB:1081640.1 | 1 | 168 |
| 924 | 2654856 | IB:1081640.1 | 1 | 247 |
| 925 | 3181917 | IB:1081640.1 | 1 | 307 |
| 926 | 2016903 | IB:1081640.1 | 1 | 262 |
| 927 | 701230984 | ZS:166646.1 | 1 | 245 |
| 928 | 700627723 | ZS:166646.1 | 1 | 250 |
| 929 | 700911955 | ZS:166646.1 | 1 | 262 |
| 930 | 701280056 | ZS:166646.1 | 1 | 267 |
| 931 | 700769886 | ZS:166646.1 | 1 | 259 |
| 932 | 700647424 | ZS:166646.1 | 2 | 176 |
| 933 | 701396532 | ZS:166646.1 | 1 | 102 |
| 934 | 700490877 | ZS:166646.1 | 1 | 272 |
| 935 | 700374582 | ZS:166646.1 | 1 | 269 |
| 936 | 700373236 | ZS:166646.1 | 3 | 145 |
| 937 | 700546735 | ZS:166646.1 | 1 | 281 |
| 938 | 701194114 | ZS:166646.1 | 1 | 237 |
| 939 | 701644894 | ZS:166646.1 | 1 | 319 |
| 940 | 700126003 | ZS:166646.1 | 3 | 90 |
| 941 | 700642857 | ZS:166646.1 | 2 | 205 |
| 942 | 701512158 | ZS:166646.1 | 3 | 268 |
| 943 | 700479909 | ZS:166646.1 | 1 | 265 |
| 944 | 700059536 | ZS:166646.1 | 1 | 296 |
| 945 | 700629237 | ZS:166646.1 | 1 | 265 |
| 946 | 701304081 | ZS:166646.1 | 1 | 210 |
| 947 | 701651203 | ZS:166646.1 | 1 | 196 |
| 948 | 700505437 | ZS:166646.1 | 1 | 268 |
| 949 | 701484590 | ZS:166646.1 | 1 | 276 |
| 950 | 701026436 | ZS:166646.1 | 1 | 240 |
| 951 | 701467315 | ZS:166646.1 | 1 | 286 |
| 952 | 701484801 | ZS:166646.1 | 1 | 232 |
| 953 | 700911350 | ZS:166646.1 | 1 | 258 |
| 954 | 700461547 | ZS:166646.1 | 1 | 202 |
| 955 | 700493031 | ZS:166646.1 | 1 | 284 |
| 956 | 701490669 | ZS:166646.1 | 1 | 271 |
| 957 | 700514441 | ZS:166646.1 | 2 | 301 |
| 958 | 701025551 | ZS:166646.1 | 1 | 274 |
| 959 | 700396312 | ZS:166646.1 | 3 | 71 |
| 960 | 701485302 | ZS:166646.1 | 3 | 294 |
| 961 | 701913515 | ZS:166646.1 | 1 | 228 |
| 962 | 701021709 | ZS:166646.1 | 1 | 153 |
| 963 | 701186432 | ZS:166646.1 | 1 | 195 |
| 964 | 700310390 | ZS:166646.1 | 1 | 301 |
| 965 | 700591163 | ZS:166646.1 | 1 | 231 |
| 966 | 701290475 | ZS:166646.1 | 1 | 133 |
| 967 | 700639812 | ZS:166646.1 | 1 | 194 |
| 969 | 701022008 | ZS:166646.1 | 1 | 181 |
| 970 | 701468265 | ZS:166646.1 | 1 | 248 |
| 971 | 700769478 | ZS:166646.1 | 1 | 260 |
| 972 | 701464269 | ZS:166646.1 | 1 | 230 |
| 973 | 701827434 | ZS:166646.1 | 7 | 293 |
| 974 | 701718036 | ZS:166646.1 | 9 | 295 |
| 975 | 700435067 | ZS:166646.1 | 3 | 327 |
| 976 | 700415992 | ZS:166646.1 | 1 | 250 |
| 977 | 700311142 | ZS:166646.1 | 1 | 284 |
| 978 | 700812651 | ZS:166646.1 | 1 | 273 |
| 979 | 701021635 | ZS:166646.1 | 1 | 251 |
| 980 | 700519425 | ZS:166646.1 | 1 | 184 |
| 981 | 701489338 | ZS:166646.1 | 1 | 278 |
| 982 | 700275567 | ZS:166646.1 | 1 | 299 |
| 983 | 4344032 | LG:2005311.5 | 2 | 114 |
| 984 | 700370485 | ZS:060925.1 | 1 | 220 |
| 985 | 2907890 | IB:1896558.1 | 1 | 291 |
| 986 | 2781237 | IB:1896558.1 | 1 | 224 |
| 987 | 700933680 | ZS:156270.1 | 1 | 201 |
| 988 | 701714807 | ZS:156270.1 | 1 | 78 |
| 989 | 700543268 | ZS:156270.1 | 1 | 135 |
| 990 | 700629946 | ZS:156270.1 | 2 | 80 |
| 991 | 700543958 | ZS:156270.1 | 1 | 248 |
| 992 | 700809939 | ZS:156270.1 | 1 | 215 |
| 993 | 700275945 | ZS:156270.1 | 1 | 74 |
| 994 | 701260994 | ZS:156270.1 | 1 | 201 |
| 995 | 701263538 | ZS:156270.1 | 1 | 258 |
| 996 | 700499733 | ZS:156270.1 | 1 | 196 |
| 997 | 700363490 | ZS:156270.1 | 2 | 166 |
| 998 | 700820713 | ZS:163272.1 | 1 | 265 |
| 999 | 700530107 | ZS:163272.1 | 2 | 268 |
| 1000 | 700819863 | ZS:163272.1 | 1 | 217 |
| 1001 | 700814470 | ZS:163272.1 | 1 | 243 |
| 1002 | 701286468 | ZS:163272.1 | 1 | 265 |
| 1003 | 700189527 | ZS:163272.1 | 1 | 303 |
| 1004 | 701286761 | ZS:163272.1 | 1 | 238 |
| 1005 | 700602464 | ZS:163272.1 | 1 | 109 |
| 1006 | 700271991 | ZS:163272.1 | 1 | 83 |
| 1007 | 700856846 | ZS:163272.1 | 1 | 268 |
| 1008 | 701910492 | ZS:163272.1 | 3 | 285 |
| 1009 | 700591303 | ZS:163272.1 | 1 | 267 |
| 1010 | 701295547 | ZS:163272.1 | 1 | 189 |
| 1011 | 700251664 | ZS:163272.1 | 1 | 219 |
| 1012 | 700061690 | ZS:163272.1 | 1 | 297 |
| 1013 | 700522555 | ZS:163272.1 | 1 | 277 |
| 1014 | 700312605 | ZS:163272.1 | 1 | 273 |
| 1015 | 700486975 | ZS:163272.1 | 1 | 88 |
| 1016 | 701280837 | ZS:163272.1 | 1 | 249 |
| 1017 | 701263345 | ZS:163272.1 | 1 | 272 |
| 1018 | 700130847 | ZS:163272.1 | 1 | 198 |
| 1019 | 701294142 | ZS:163272.1 | 1 | 248 |
| 1020 | 700127864 | ZS:163272.1 | 1 | 262 |
| 1021 | 700419566 | ZS:163272.1 | 1 | 275 |
| 1022 | 700298707 | ZS:163272.1 | 1 | 266 |
| 1023 | 700521683 | ZS:163272.1 | 1 | 283 |
| 1024 | 700489028 | ZS:163272.1 | 1 | 277 |
| 1025 | 700821256 | ZS:163272.1 | 1 | 230 |
| 1026 | 700297908 | ZS:163272.1 | 1 | 297 |
| 1027 | 700129307 | ZS:163272.1 | 2 | 249 |
| 1028 | 700417150 | ZS:163272.1 | 1 | 272 |
| 1029 | 700486715 | ZS:163272.1 | 2 | 245 |
| 1030 | 700268744 | ZS:163272.1 | 1 | 284 |
| 1031 | 700174616 | ZS:163272.1 | 1 | 250 |
| 1032 | 700489346 | ZS:163272.1 | 1 | 280 |
| 1033 | 701270561 | ZS:163272.1 | 1 | 257 |
| 1034 | 701300219 | ZS:163272.1 | 1 | 195 |
| 1035 | 700522758 | ZS:163272.1 | 1 | 147 |
| 1036 | 700068332 | ZS:163272.1 | 1 | 174 |
| 1037 | 700525582 | ZS:163272.1 | 1 | 177 |
| 1038 | 700300221 | ZS:163272.1 | 1 | 287 |
| 1039 | 700533847 | ZS:163272.1 | 1 | 286 |
| 1040 | 701273613 | ZS:163272.1 | 1 | 228 |
| 1041 | 701255660 | ZS:163272.1 | 1 | 256 |
| 1042 | 2747183 | IB:2033867.1 | 1 | 130 |
| 1043 | 996393 | IB:2033867.1 | 1 | 60 |
| 1044 | 1447832 | IB:2033867.1 | 1 | 57 |
| 1045 | 286060 | IB:2033867.1 | 24 | 94 |
| 1046 | 3980387 | IB:2106419.1 | 1 | 206 |
| 1047 | 700250549 | ZS:019755.1 | 1 | 282 |
| 1048 | 700123628 | ZS:019755.1 | 1 | 233 |
| 1049 | 2938247 | IB:2024261.1 | 20 | 262 |
| 1050 | 22377 | IB:2024261.2 | 1 | 268 |
| 1051 | 1925075 | IB:2024261.2 | 1 | 277 |
| 1052 | 1364475 | IB:2024261.2 | 1 | 203 |
| 1053 | 3438723 | IB:2024261.2 | 1 | 248 |
| 1054 | 2303842 | IB:2024261.2 | 1 | 255 |
| 1055 | 691479 | IB:2024261.2 | 1 | 179 |
| 1056 | 2097617 | IB:2024261.2 | 1 | 171 |
| 1057 | 680352 | IB:2024261.2 | 1 | 220 |
| 1058 | 996586 | IB:2024261.2 | 1 | 245 |
| 1059 | 1476048 | IB:2024261.2 | 1 | 203 |
| 1060 | 2153735 | IB:2024261.2 | 1 | 265 |
| 1061 | 2397557 | IB:2024261.2 | 1 | 228 |
| 1062 | 2171748 | IB:2024261.2 | 1 | 202 |
| 1063 | 3929904 | IB:2024261.2 | 1 | 92 |
| 1064 | 2734931 | IB:2024261.2 | 1 | 211 |
| 1065 | 2582411 | IB:2024261.2 | 1 | 306 |
| 1066 | 3089081 | IB:2024261.2 | 1 | 274 |
| 1067 | 663716 | IB:2024261.2 | 1 | 245 |
| 1068 | 2278413 | IB:2024261.2 | 1 | 276 |
| 1069 | 24810 | IB:2024261.2 | 1 | 303 |
| 1070 | 2591180 | IB:2024261.2 | 1 | 250 |
| 1071 | 4907489 | IB:2024261.2 | 1 | 77 |
| 1072 | 259698 | IB:2024261.2 | 1 | 364 |
| 1073 | 21859 | IB:2024261.2 | 1 | 256 |
| 1074 | 2618859 | IB:2024261.2 | 1 | 257 |
| 1075 | 1450971 | IB:2024261.2 | 1 | 111 |
| 1076 | 3079454 | IB:2024261.2 | 1 | 307 |
| 1077 | 1493650 | IB:2024261.2 | 1 | 207 |
| 1078 | 2370759 | IB:2024261.2 | 1 | 237 |
| 1079 | 2290353 | IB:2024261.2 | 1 | 249 |
| 1080 | 2275525 | IB:2024261.2 | 1 | 262 |
| 1081 | 2287631 | IB:2024261.2 | 1 | 239 |
| 1082 | 2986658 | IB:2024261.2 | 2 | 233 |
| 1083 | 2452912 | IB:2024261.2 | 1 | 238 |
| 1084 | 2509602 | IB:2024261.2 | 1 | 149 |
| 1085 | 3467240 | IB:2024261.2 | 2 | 242 |
| 1086 | 3079537 | IB:2024261.2 | 1 | 148 |
| 1087 | 1271327 | IB:2024261.2 | 1 | 168 |
| 1088 | 452363 | IB:2024261.2 | 1 | 229 |
| 1089 | 3029510 | IB:2024261.2 | 1 | 267 |
| 1090 | 2838736 | IB:2024261.2 | 1 | 246 |
| 1091 | 2503774 | IB:2024261.2 | 1 | 158 |
| 1092 | 683266 | IB:2024261.2 | 1 | 147 |
| 1093 | 679322 | IB:2024261.2 | 1 | 119 |
| 1094 | 2941393 | IB:2024261.2 | 1 | 139 |
| 1095 | 1517789 | IB:2024261.2 | 1 | 202 |
| 1096 | 217 | IB:2024261.2 | 8 | 277 |
| 1097 | 2697521 | IB:2024261.2 | 1 | 265 |
| 1098 | 3279421 | IB:2024261.2 | 1 | 236 |
| 1099 | 54555 | IB:2024261.2 | 1 | 164 |
| 1100 | 2742812 | IB:2024261.2 | 1 | 196 |
| 1101 | 2583990 | IB:2024261.2 | 1 | 260 |
| 1102 | 2349158 | IB:2024261.2 | 1 | 193 |
| 1103 | 3567447 | IB:2024261.2 | 1 | 155 |
| 1104 | 2654142 | IB:2024261.2 | 3 | 291 |
| 1105 | 266368 | IB:2024261.2 | 4 | 356 |
| 1106 | 654402 | IB:2024261.2 | 1 | 285 |
| 1107 | 21552 | IB:2024261.2 | 1 | 253 |
| 1108 | 956044 | IB:2024261.2 | 1 | 238 |
| 1109 | 598239 | IB:2024261.2 | 1 | 238 |
| 1110 | 2809547 | IB:2024261.2 | 1 | 252 |
| 1111 | 1475663 | IB:2024261.2 | 1 | 191 |
| 1112 | 2445935 | IB:2024261.2 | 1 | 226 |
| 1113 | 1513731 | IB:2024261.2 | 1 | 196 |
| 1114 | 2586284 | IB:2024261.2 | 1 | 219 |
| 1115 | 2699720 | IB:2024261.2 | 1 | 264 |
| 1116 | 479348 | IB:2024261.2 | 1 | 286 |
| 1117 | 2752584 | IB:2024261.2 | 1 | 261 |
| 1118 | 1850751 | IB:2024261.2 | 1 | 135 |
| 1119 | 2133163 | IB:2024261.2 | 1 | 244 |
| 1120 | 2154178 | IB:2024261.2 | 1 | 170 |
| 1121 | 700179763 | ZS:173316.1 | 1 | 245 |
| 1122 | 700776044 | ZS:173316.2 | 1 | 257 |
| 1123 | 700860742 | ZS:173316.2 | 1 | 259 |
| 1124 | 700862233 | ZS:173316.2 | 4 | 238 |
| 1125 | 1700421 | IB:2094484.7 | 1 | 189 |
| 1126 | 2914593 | IB:2094484.7 | 1 | 198 |
| 1127 | 1878873 | IB:2094484.7 | 1 | 260 |
| 1128 | 1391107 | IB:2094484.7 | 1 | 241 |
| 1129 | 2964175 | IB:2094484.7 | 1 | 285 |
| 1130 | 6313477 | LG:1996166.4 | 2 | 386 |
| 1131 | 6613886 | LG:2022251.5 | 1 | 197 |
| 1132 | 6270862 | LG:2022251.6 | 1 | 606 |
| 1133 | 5814585 | LG:2022251.6 | 1 | 232 |
| 1134 | 6513219 | LG:2022251.6 | 1 | 456 |
| 1135 | 6421324 | LG:2022251.6 | 1 | 393 |
| 1136 | 5864057 | LG:2022251.6 | 5 | 281 |
| 1137 | 6401763 | LG:2022251.6 | 1 | 246 |
| 1138 | 6378821 | LG:2022251.6 | 3 | 275 |
| 1139 | 6496712 | LG:2022251.6 | 1 | 568 |
| 1140 | 6419875 | LG:2022251.6 | 1 | 193 |
| 1141 | 5822327 | LG:2022251.6 | 1 | 278 |
| 1142 | 5813672 | LG:2022251.6 | 1 | 313 |
| 1143 | 6408660 | LG:2022251.7 | 1 | 231 |
| 1144 | 4518370 | LG:2022251.7 | 3 | 241 |
| 1145 | 6517553 | LG:2022251.7 | 1 | 479 |
| 1146 | 6136188 | LG:2022251.7 | 1 | 276 |
| 1147 | 6410975 | LG:2022251.7 | 1 | 308 |
| 1148 | 5823548 | LG:2022251.7 | 17 | 212 |
| 1149 | 6369723 | LG:2022251.7 | 1 | 569 |
| 1150 | 6014804 | LG:2022251.7 | 1 | 186 |
| 1151 | 6398907 | LG:2022251.7 | 3 | 240 |
| 1152 | 6568514 | LG:2022251.7 | 1 | 455 |
| 1153 | 5819602 | LG:2022251.7 | 107 | 324 |
| 1154 | 6399030 | LG:2022251.7 | 1 | 289 |
| 1155 | 6591075 | LG:2022251.7 | 1 | 350 |
| 1156 | 6257326 | LG:2022251.7 | 3 | 270 |
| 1157 | 5698802 | LG:2022251.7 | 1 | 235 |
| 1158 | 701229180 | ZS:158013.1 | 1 | 240 |
| 1159 | 700934730 | ZS:158013.1 | 1 | 76 |
| 1160 | 700948690 | ZS:158013.1 | 1 | 281 |
| 1161 | 700295204 | ZS:158013.1 | 4 | 214 |
| 1162 | 701277731 | ZS:158013.1 | 1 | 233 |
| 1163 | 701334863 | ZS:158013.1 | 1 | 213 |
| 1164 | 701274943 | ZS:158013.1 | 1 | 148 |
| 1165 | 701275514 | ZS:158013.1 | 1 | 154 |
| 1166 | 700127275 | ZS:158013.1 | 1 | 93 |
| 1167 | 701333270 | ZS:158013.1 | 1 | 244 |
| 1168 | 701944168 | ZS:168075.2 | 1 | 210 |
| 1169 | 701466250 | ZS:168075.2 | 2 | 273 |
| 1170 | 701944268 | ZS:168075.2 | 1 | 286 |
| 1171 | 701258551 | ZS:168075.2 | 1 | 253 |
| 1172 | 701616185 | ZS:168075.2 | 1 | 333 |
| 1173 | 700141178 | ZS:168075.2 | 1 | 240 |
| 1174 | 701734388 | ZS:168075.2 | 1 | 304 |
| 1175 | 701725735 | ZS:168075.2 | 1 | 319 |
| 1176 | 701339880 | ZS:168075.2 | 1 | 81 |
| 1177 | 701475064 | ZS:168075.2 | 1 | 266 |
| 1178 | 700303823 | ZS:168075.2 | 2 | 187 |
| 1179 | 700524067 | ZS:168075.2 | 1 | 256 |
| 1180 | 701880885 | ZS:168075.2 | 1 | 283 |
| 1181 | 701396970 | ZS:168075.2 | 1 | 188 |
| 1182 | 700132732 | ZS:168075.2 | 1 | 258 |
| 1183 | 700140788 | ZS:168075.2 | 1 | 236 |
| 1184 | 2234427 | IB:1448568.1 | 1 | 263 |
| 1185 | 2242421 | IB:1448568.1 | 1 | 244 |
| 1186 | 2703885 | IB:1537014.1 | 1 | 174 |
| 1187 | 3328680 | IB:1537014.1 | 1 | 271 |
| 1188 | 389504 | IB:1086288.1 | 1 | 221 |
| 1189 | 343775 | IB:1086288.1 | 1 | 94 |
| 1190 | 2749717 | IB:2022623.10 | 1 | 239 |
| 1191 | 2795118 | IB:2022623.10 | 1 | 313 |
| 1192 | 2544029 | IB:2022623.10 | 1 | 251 |
| 1193 | 1488916 | IB:2022623.10 | 1 | 211 |
| 1194 | 2482284 | IB:2022623.10 | 1 | 222 |
| 1195 | 1591308 | IB:2022623.10 | 1 | 212 |
| 1196 | 2748126 | IB:2022623.10 | 1 | 237 |
| 1197 | 2477774 | IB:2022623.10 | 1 | 241 |
| 1198 | 2542849 | IB:2022623.10 | 1 | 221 |
| 1199 | 3973829 | IB:2022623.10 | 1 | 246 |
| 1200 | 1699889 | IB:2022623.10 | 1 | 235 |
| 1201 | 1475835 | IB:2022623.10 | 1 | 187 |
| 1202 | 4346925 | IB:2022623.10 | 1 | 251 |
| 1203 | 854372 | IB:2022623.10 | 1 | 251 |
| 1204 | 1489193 | IB:2022623.10 | 1 | 208 |
| 1205 | 813961 | IB:2022623.10 | 1 | 67 |
| 1206 | 676879 | IB:2022623.10 | 1 | 282 |
| 1207 | 2883157 | IB:2022623.10 | 1 | 248 |
| 1208 | 1930333 | IB:2022623.10 | 1 | 265 |
| 1209 | 2465467 | IB:2022623.10 | 1 | 233 |
| 1210 | 3979618 | IB:2022623.10 | 1 | 297 |
| 1211 | 1548750 | IB:2022623.10 | 1 | 177 |
| 1212 | 2482006 | IB:2022623.10 | 1 | 235 |
| 1213 | 3972034 | IB:2022623.10 | 1 | 279 |
| 1214 | 1302173 | IB:2022623.10 | 1 | 293 |
| 1215 | 1754053 | IB:2022623.10 | 1 | 246 |
| 1216 | 1805622 | IB:2022623.10 | 1 | 252 |
| 1217 | 355076 | IB:2022623.10 | 1 | 222 |
| 1218 | 1445360 | IB:2022623.10 | 1 | 240 |
| 1219 | 1915970 | IB:2022623.10 | 1 | 279 |
| 1220 | 491397 | IB:2022623.10 | 1 | 237 |
| 1221 | 1405153 | IB:2022623.10 | 1 | 264 |
| 1222 | 1423720 | IB:2022623.10 | 1 | 219 |
| 1223 | 2875708 | IB:2022623.10 | 1 | 283 |
| 1224 | 550170 | IB:2022623.10 | 1 | 249 |
| 1225 | 2278238 | IB:2022623.10 | 1 | 255 |
| 1226 | 1332454 | IB:2022623.10 | 1 | 235 |
| 1227 | 155152 | IB:2022623.10 | 1 | 193 |
| 1228 | 1332142 | IB:2022623.10 | 1 | 99 |
| 1229 | 2683259 | IB:2022623.10 | 1 | 310 |
| 1230 | 2827925 | IB:2022623.10 | 1 | 258 |
| 1231 | 2862812 | IB:2022623.10 | 1 | 276 |
| 1232 | 2122664 | IB:2022623.10 | 1 | 268 |
| 1233 | 2554840 | IB:2022623.10 | 1 | 245 |
| 1234 | 2630572 | IB:2022623.10 | 1 | 254 |
| 1235 | 465062 | IB:2022623.10 | 1 | 221 |
| 1236 | 2908988 | IB:2022623.10 | 1 | 231 |
| 1237 | 1870177 | IB:2022623.10 | 1 | 227 |
| 1238 | 3340773 | IB:2022623.10 | 1 | 241 |
| 1239 | 938004 | IB:2022623.10 | 1 | 262 |
| 1240 | 2848865 | IB:2022623.10 | 1 | 268 |
| 1241 | 2439949 | IB:2022623.10 | 1 | 103 |
| 1242 | 1414913 | IB:2022623.10 | 1 | 135 |
| 1243 | 2720175 | IB:2022623.10 | 1 | 93 |
| 1244 | 2685714 | IB:2022623.10 | 1 | 254 |
| 1245 | 2371533 | IB:2022623.10 | 1 | 217 |
| 1246 | 3088650 | IB:2022623.10 | 1 | 253 |
| 1247 | 2152591 | IB:2022623.10 | 1 | 226 |
| 1248 | 780556 | IB:2022623.10 | 1 | 162 |
| 1249 | 3947580 | IB:2022623.10 | 1 | 280 |
| 1250 | 3981972 | IB:2022623.10 | 1 | 294 |
| 1251 | 2659340 | IB:2022623.10 | 1 | 235 |
| 1252 | 937943 | IB:2022623.10 | 1 | 304 |
| 1253 | 1320953 | IB:2022623.10 | 1 | 240 |
| 1254 | 3972523 | IB:2022623.10 | 1 | 278 |
| 1255 | 1871348 | IB:2022623.10 | 1 | 125 |
| 1256 | 2660893 | IB:2022623.10 | 1 | 229 |
| 1257 | 2665670 | IB:2022623.10 | 1 | 247 |
| 1258 | 3180353 | IB:2022623.10 | 1 | 296 |
| 1259 | 1484296 | IB:2022623.10 | 1 | 262 |
| 1260 | 701607583 | ZS:156579.1 | 1 | 303 |
| 1261 | 700372543 | ZS:156579.1 | 1 | 268 |
| 1262 | 700781722 | ZS:156579.1 | 1 | 266 |
| 1263 | 701649506 | ZS:156579.1 | 1 | 261 |
| 1264 | 700768474 | ZS:156579.1 | 1 | 251 |
| 1265 | 700366914 | ZS:156579.1 | 1 | 263 |
| 1266 | 700274636 | ZS:156579.1 | 1 | 199 |
| 1267 | 700328048 | ZS:156579.1 | 1 | 296 |
| 1268 | 700485657 | ZS:156579.1 | 1 | 279 |
| 1269 | 700635667 | ZS:156579.1 | 1 | 254 |
| 1270 | 701232896 | ZS:156579.1 | 1 | 143 |
| 1271 | 701580093 | ZS:156579.1 | 1 | 350 |
| 1272 | 700519840 | ZS:156579.1 | 1 | 274 |
| 1273 | 700627492 | ZS:156579.1 | 1 | 249 |
| 1274 | 700530153 | ZS:156579.1 | 1 | 280 |
| 1275 | 701419250 | ZS:156579.1 | 1 | 221 |
| 1276 | 700636665 | ZS:156579.1 | 1 | 244 |
| 1277 | 701343275 | ZS:156579.1 | 1 | 251 |
| 1278 | 701319684 | ZS:156579.1 | 1 | 138 |
| 1279 | 700766587 | ZS:156579.1 | 1 | 260 |
| 1280 | 701392692 | ZS:156579.1 | 1 | 246 |
| 1281 | 700785820 | ZS:156579.1 | 1 | 259 |
| 1282 | 700914592 | ZS:156579.1 | 1 | 237 |
| 1283 | 700625377 | ZS:156579.1 | 1 | 246 |
| 1284 | 700785521 | ZS:156579.1 | 1 | 244 |
| 1285 | 700306832 | ZS:156579.1 | 1 | 258 |
| 1286 | 700626941 | ZS:156579.1 | 1 | 78 |
| 1287 | 700276565 | ZS:156579.1 | 1 | 201 |
| 1288 | 700314659 | ZS:156579.1 | 1 | 283 |
| 1289 | 700420110 | ZS:156579.1 | 1 | 157 |
| 1290 | 700246776 | ZS:156579.1 | 1 | 161 |
| 1291 | 700288432 | ZS:156579.1 | 1 | 251 |
| 1292 | 701519417 | ZS:156579.1 | 1 | 284 |
| 1293 | 700629369 | ZS:156579.1 | 1 | 260 |
| 1294 | 701025922 | ZS:156579.1 | 1 | 262 |
| 1295 | 700631958 | ZS:156579.1 | 1 | 268 |
| 1296 | 700636674 | ZS:156579.1 | 1 | 247 |
| 1297 | 700914122 | ZS:156579.1 | 1 | 277 |
| 1298 | 701024824 | ZS:156579.1 | 1 | 158 |
| 1299 | 700910639 | ZS:156579.1 | 1 | 243 |
| 1300 | 2328033 | IB:2094098.6 | 1 | 255 |
| 1301 | 579187 | IB:2095659.1 | 1 | 268 |
| 1302 | 700534404 | ZS:173892.1 | 1 | 287 |
| 1303 | 701344942 | ZS:173892.3 | 1 | 282 |
| 1304 | 700525076 | ZS:173892.3 | 4 | 104 |
| 1305 | 701292649 | ZS:173892.3 | 1 | 176 |
| 1306 | 701654201 | ZS:173892.3 | 3 | 237 |
| 1307 | 700289551 | ZS:173892.3 | 1 | 313 |
| 1308 | 700415338 | ZS:173892.3 | 1 | 114 |
| 1309 | 700538084 | ZS:173892.3 | 1 | 265 |
| 1310 | 700485031 | ZS:173892.3 | 1 | 225 |
| 1311 | 701650221 | ZS:173892.3 | 2 | 294 |
| 1312 | 701655217 | ZS:173892.3 | 2 | 120 |
| 1313 | 700305954 | ZS:173892.3 | 3 | 91 |
| 1314 | 701468078 | ZS:173892.3 | 5 | 222 |
| 1315 | 6027904 | LG:2005311.6 | 1 | 288 |
| 1316 | 6593314 | LG:2005311.6 | 1 | 499 |
| 1317 | 6307844 | LG:2005311.6 | 1 | 233 |
| 1318 | 6593514 | LG:2005311.6 | 5 | 547 |
| 1319 | 5303918 | LG:2005311.6 | 1 | 272 |
| 1320 | 5885852 | LG:2005311.7 | 3 | 248 |
| 1321 | 6523764 | LG:2005311.7 | 1 | 235 |
| 1322 | 5993818 | LG:2005311.7 | 1 | 297 |
| 1323 | 5882855 | LG:2005311.7 | 3 | 240 |
| 1324 | 6114074 | LG:2005311.7 | 1 | 267 |
| 1325 | 3567250 | LG:2005311.7 | 1 | 107 |
| 1326 | 6114081 | LG:2005311.7 | 1 | 267 |
| 1327 | 5943729 | LG:2005311.7 | 1 | 268 |
| 1328 | 4211225 | LG:2005311.7 | 1 | 286 |
| 1329 | 5885814 | LG:2005311.7 | 3 | 255 |
| 1330 | 6605876 | LG:2005311.7 | 1 | 475 |
| 1331 | 5697593 | LG:2005311.7 | 2 | 61 |
| 1332 | 4209022 | LG:2005311.7 | 1 | 280 |
| 1333 | 6364143 | LG:2005311.7 | 3 | 90 |
| 1334 | 3780874 | LG:2005311.7 | 3 | 284 |
| 1335 | 5886691 | LG:2005311.7 | 1 | 256 |
| 1336 | 6512389 | LG:2005311.7 | 1 | 554 |
| 1337 | 4516729 | LG:2005311.7 | 1 | 251 |
| 1338 | 6522528 | LG:2005311.7 | 1 | 535 |
| 1339 | 6299317 | LG:2005311.7 | 1 | 269 |
| 1340 | 6354938 | LG:2005311.7 | 1 | 271 |
| 1341 | 6411611 | LG:2005311.7 | 2 | 511 |
| 1342 | 6271241 | LG:2005311.7 | 1 | 549 |
| 1343 | 4438756 | LG:2005311.7 | 1 | 278 |
| 1344 | 4438771 | LG:2005311.7 | 2 | 270 |
| 1345 | 4443584 | LG:2005311.7 | 1 | 136 |
| 1346 | 6098582 | LG:2005311.7 | 11 | 278 |
| 1347 | 6361222 | LG:2005311.7 | 1 | 466 |
| 1348 | 5883291 | LG:2005311.7 | 1 | 307 |
| 1349 | 6366607 | LG:2005311.7 | 5 | 259 |
| 1350 | 5954210 | LG:2005311.7 | 6 | 276 |
| 1351 | 4515822 | LG:2005311.7 | 1 | 256 |
| 1352 | 700488315 | ZS:156325.3 | 1 | 288 |
| 1353 | 701728973 | ZS:156325.3 | 1 | 280 |
| 1354 | 700367922 | ZS:156325.3 | 1 | 136 |
| 1355 | 700486083 | ZS:156325.3 | 3 | 200 |
| 1356 | 701484604 | ZS:156325.3 | 1 | 284 |
| 1357 | 6480126 | LG:2018794.6 | 1 | 593 |
| 1358 | 6373901 | LG:2018794.6 | 1 | 291 |
| 1359 | 5890253 | LG:2018794.6 | 1 | 258 |
| 1360 | 6546449 | LG:2018794.6 | 1 | 161 |
| 1361 | 6424874 | LG:2018794.6 | 1 | 555 |
| 1362 | 6176089 | LG:2018794.6 | 1 | 264 |
| 1363 | 5885588 | LG:2018794.6 | 1 | 256 |
| 1364 | 5884115 | LG:2018794.6 | 1 | 250 |
| 1365 | 6431732 | LG:2018794.6 | 1 | 71 |
| 1366 | 6319383 | LG:2018794.6 | 1 | 273 |
| 1367 | 6006445 | LG:2018794.6 | 1 | 281 |
| 1368 | 5714842 | LG:2018794.6 | 1 | 266 |
| 1369 | 6153505 | LG:2018794.6 | 162 | 245 |
| 1370 | 6245935 | LG:2018794.6 | 1 | 521 |
| 1371 | 6306763 | LG:2018794.6 | 1 | 259 |
| 1372 | 5945146 | LG:2018794.6 | 1 | 282 |
| 1373 | 5884982 | LG:2018794.6 | 1 | 233 |
| 1374 | 6322160 | LG:2018794.6 | 1 | 128 |
| 1375 | 3154287 | LG:2018794.6 | 3 | 230 |
| 1376 | 6375238 | LG:2018794.6 | 1 | 263 |
| 1377 | 6246035 | LG:2018794.6 | 1 | 539 |
| 1378 | 6537280 | LG:2018794.6 | 1 | 320 |
| 1379 | 1319812 | IB:2080205.1 | 1 | 224 |
| 1380 | 1389252 | IB:2080205.1 | 1 | 222 |
| 1381 | 886511 | IB:2094678.10 | 1 | 142 |
| 1382 | 3275752 | IB:2094678.10 | 1 | 251 |
| 1383 | 767583 | IB:2094678.10 | 1 | 245 |
| 1384 | 2822820 | IB:2094678.10 | 1 | 309 |
| 1385 | 2231189 | IB:2094678.10 | 1 | 237 |
| 1386 | 2625106 | IB:2094678.10 | 1 | 231 |
| 1387 | 2823682 | IB:2094678.10 | 1 | 278 |
| 1388 | 500 | IB:2022421.8 | 1 | 363 |
| 1389 | 682041 | IB:2022421.8 | 1 | 230 |
| 1390 | 101255 | IB:2022421.8 | 11 | 127 |
| 1391 | 219448 | IB:2022421.8 | 1 | 194 |
| 1392 | 303290 | IB:2022421.8 | 6 | 232 |
| 1393 | 207382 | IB:2022421.8 | 1 | 210 |
| 1394 | 580332 | IB:2022421.8 | 1 | 228 |
| 1395 | 23424 | IB:2022421.8 | 1 | 271 |
| 1396 | 2636808 | IB:2022421.8 | 1 | 250 |
| 1397 | 895100 | IB:2022421.8 | 1 | 258 |
| 1398 | 988478 | IB:2022421.8 | 1 | 59 |
| 1399 | 927899 | IB:2022421.8 | 1 | 119 |
| 1400 | 215284 | IB:2022421.8 | 1 | 330 |
| 1401 | 21437 | IB:2022421.8 | 3 | 315 |
| 1402 | 24011 | IB:2022421.8 | 3 | 278 |
| 1403 | 21857 | IB:2022421.8 | 1 | 257 |
| 1404 | 21630 | IB:2022421.8 | 1 | 302 |
| 1405 | 1818131 | IB:2022421.8 | 1 | 145 |
| 1406 | 2663627 | IB:2047198.1 | 1 | 238 |
| 1407 | 5920138 | LG:2021282.7 | 1 | 308 |
| 1408 | 1753287 | IB:2072495.1 | 1 | 217 |
| 1409 | 1812515 | IB:2072495.1 | 1 | 239 |
| 1410 | 1710659 | IB:2072495.1 | 1 | 114 |
| 1411 | 176197 | IB:2072495.1 | 1 | 376 |
| 1412 | 1694075 | IB:2072495.1 | 1 | 212 |
| 1413 | 1753396 | IB:2072495.1 | 1 | 191 |
| 1414 | 1753376 | IB:2072495.1 | 1 | 237 |
| 1415 | 2635071 | IB:2025662.6 | 1 | 251 |
| 1416 | 86657 | IB:2025662.8 | 1 | 201 |
| 1417 | 139171 | IB:2025662.8 | 5 | 59 |
| 1418 | 5878526 | LG:1925055.3 | 1 | 277 |
| 1419 | 6600271 | LG:1925055.3 | 1 | 524 |
| 1420 | 5561109 | LG:1925055.3 | 1 | 213 |
| 1421 | 5703148 | LG:1925055.3 | 2 | 282 |
| 1422 | 4443746 | LG:1925055.3 | 2 | 185 |
| 1423 | 6428594 | LG:1925055.3 | 1 | 214 |
| 1424 | 4343561 | LG:1925055.3 | 1 | 285 |
| 1425 | 3021152 | LG:1925055.3 | 1 | 280 |
| 1426 | 6213466 | LG:1925055.3 | 3 | 265 |
| 1427 | 6376812 | LG:1925055.3 | 3 | 276 |
| 1428 | 5700295 | LG:1925055.3 | 1 | 274 |
| 1429 | 6364679 | LG:1925055.3 | 1 | 288 |
| 1430 | 6489032 | LG:1925055.3 | 1 | 488 |
| 1431 | 6378287 | LG:1925055.3 | 2 | 275 |
| 1432 | 6349775 | LG:1925055.3 | 1 | 323 |
| 1433 | 6379980 | LG:1925055.3 | 4 | 285 |
| 1434 | 4516928 | LG:1925055.3 | 1 | 213 |
| 1435 | 6412344 | LG:1925055.3 | 1 | 533 |
| 1436 | 4518475 | LG:1925055.3 | 1 | 127 |
| 1437 | 5704048 | LG:1925055.3 | 1 | 262 |
| 1438 | 6363156 | LG:1925055.3 | 1 | 572 |
| 1439 | 6521229 | LG:1925055.3 | 1 | 109 |
| 1440 | 6455607 | LG:1925055.3 | 1 | 505 |
| 1441 | 6598793 | LG:1925055.3 | 1 | 490 |
| 1442 | 6208789 | LG:1925055.3 | 1 | 303 |
| 1443 | 6498438 | LG:1925055.3 | 1 | 531 |
| 1444 | 3899830 | LG:1925055.3 | 1 | 292 |
| 1445 | 4441007 | LG:1925055.3 | 1 | 245 |
| 1446 | 4668358 | LG:1925055.3 | 1 | 179 |
| 1447 | 6208589 | LG:1925055.3 | 4 | 84 |
| 1448 | 3899623 | LG:1925055.3 | 1 | 263 |
| 1449 | 4440405 | LG:1925055.3 | 1 | 229 |
| 1450 | 6491681 | LG:1925055.3 | 2 | 244 |
| 1451 | 4443438 | LG:1925055.3 | 1 | 267 |
| 1452 | 6208777 | LG:1925055.3 | 1 | 279 |
| 1453 | 6513632 | LG:1925055.3 | 1 | 491 |
| 1454 | 6431741 | LG:1925055.3 | 1 | 389 |
| 1455 | 4438923 | LG:1925055.3 | 1 | 273 |
| 1456 | 3023596 | LG:1925055.3 | 1 | 289 |
| 1457 | 6513532 | LG:1925055.3 | 1 | 421 |
| 1458 | 6023712 | LG:1925055.3 | 1 | 284 |
| 1459 | 3022286 | LG:1925055.3 | 1 | 283 |
| 1460 | 6598639 | LG:1925055.3 | 1 | 535 |
| 1461 | 5692805 | LG:1925055.3 | 1 | 215 |
| 1462 | 5702665 | LG:1925055.3 | 1 | 257 |
| 1463 | 5562537 | LG:1925055.3 | 1 | 259 |
| 1464 | 6169636 | LG:1925055.3 | 1 | 148 |
| 1465 | 6376628 | LG:1925055.3 | 4 | 269 |
| 1466 | 6023337 | LG:1925055.3 | 1 | 289 |
| 1467 | 6384872 | LG:1925055.3 | 1 | 269 |
| 1468 | 6363655 | LG:1925055.3 | 1 | 520 |
| 1469 | 6010162 | LG:1925055.3 | 1 | 116 |
| 1470 | 4517845 | LG:1925055.3 | 1 | 249 |
| 1471 | 3019790 | LG:1925055.3 | 1 | 270 |
| 1472 | 1455594 | IB:2075852.1 | 1 | 243 |
| 1473 | 1433063 | IB:2075852.1 | 1 | 237 |
| 1474 | 6422373 | LG:2021282.8 | 1 | 351 |
| 1475 | 6421660 | LG:2021282.8 | 1 | 364 |
| 1476 | 6298033 | LG:2021282.8 | 3 | 249 |
| 1477 | 4121984 | LG:2021282.8 | 1 | 280 |
| 1478 | 6134672 | LG:2021282.8 | 2 | 294 |
| 1479 | 6421768 | LG:2021282.8 | 1 | 391 |
| 1480 | 6100330 | LG:2021282.8 | 1 | 283 |
| 1481 | 6559430 | LG:2021282.8 | 1 | 554 |
| 1482 | 5873539 | LG:2021282.8 | 1 | 293 |
| 1483 | 6053960 | LG:2021282.8 | 1 | 551 |
| 1484 | 6426224 | LG:2021282.8 | 1 | 407 |
| 1485 | 6310337 | LG:2021282.3 | 1 | 568 |
| 1486 | 715790 | IB:2025662.10 | 3 | 270 |
| 1487 | 2782796 | IB:2025662.10 | 1 | 84 |
| 1488 | 2822929 | IB:2025662.10 | 1 | 269 |
| 1489 | 2096038 | IB:2025662.10 | 1 | 216 |
| 1490 | 2219006 | IB:2025662.10 | 1 | 85 |
| 1491 | 89474 | IB:2025662.10 | 1 | 181 |
| 1492 | 714309 | IB:2025662.10 | 10 | 291 |
| 1493 | 715581 | IB:2025662.10 | 1 | 60 |
| 1494 | 2085627 | IB:2025662.10 | 1 | 270 |
| 1495 | 88118 | IB:2025662.10 | 1 | 232 |
| 1496 | 2815429 | IB:2025662.10 | 1 | 328 |
| 1497 | 760074 | IB:2025662.10 | 3 | 274 |
| 1498 | 2025847 | IB:1925055.3 | 1 | 239 |
| 1499 | 3343173 | IB:1925055.3 | 2 | 256 |
| 1500 | 274309 | IB:1925055.3 | 8 | 340 |
| 1501 | 2601745 | IB:1925055.3 | 1 | 202 |
| 1502 | 2892508 | IB:1925055.3 | 1 | 266 |
| 1503 | 1362055 | IB:1925055.3 | 1 | 255 |
| 1504 | 2460609 | IB:1925055.3 | 1 | 236 |
| 1505 | 2715415 | IB:1925055.3 | 1 | 245 |
| 1506 | 3364921 | IB:1925055.3 | 1 | 209 |
| 1507 | 606092 | IB:1925055.3 | 1 | 260 |
| 1508 | 274308 | IB:1925055.3 | 4 | 355 |
| 1509 | 3358609 | IB:1925055.3 | 1 | 275 |
| 1510 | 2640032 | IB:1925055.3 | 1 | 236 |
| 1511 | 2731063 | IB:1925055.5 | 1 | 259 |
| 1512 | 2184244 | IB:1925055.5 | 1 | 273 |
| 1513 | 3204456 | IB:1925055.5 | 1 | 294 |
| 1514 | 849715 | IB:1925055.5 | 1 | 234 |
| 1515 | 2637127 | IB:1925055.5 | 1 | 263 |
| 1516 | 2531872 | IB:1925055.5 | 1 | 256 |
| 1517 | 2466966 | IB:1925055.5 | 1 | 233 |
| 1518 | 855636 | IB:1925055.5 | 1 | 240 |
| 1519 | 3200905 | IB:1925055.5 | 1 | 283 |
| 1520 | 3045769 | IB:1925055.5 | 1 | 148 |
| 1521 | 205471 | IB:1925055.5 | 10 | 210 |
| 1522 | 2603222 | IB:1925055.5 | 1 | 276 |
| 1523 | 3216147 | IB:1925055.5 | 1 | 272 |
| 1524 | 3345777 | IB:1925055.5 | 1 | 253 |
| 1525 | 2296070 | IB:1925055.5 | 1 | 255 |
| 1526 | 2210959 | IB:1925055.5 | 1 | 262 |
| 1527 | 1721127 | IB:1925055.5 | 1 | 197 |
| 1528 | 546344 | IB:1925055.5 | 1 | 219 |
| 1529 | 3685024 | IB:1925055.5 | 1 | 316 |
| 1530 | 3044734 | IB:1925055.5 | 1 | 288 |
| 1531 | 1259249 | IB:1925055.5 | 1 | 242 |
| 1532 | 2772012 | IB:1925055.5 | 1 | 248 |
| 1533 | 359806 | IB:1925055.5 | 1 | 212 |
| 1534 | 850468 | IB:1925055.5 | 1 | 189 |
| 1535 | 1814906 | IB:1925055.5 | 1 | 250 |
| 1536 | 3949616 | IB:1925055.5 | 1 | 148 |
| 1537 | 2274011 | IB:1925055.5 | 1 | 275 |
| 1538 | 350870 | IB:1925055.5 | 7 | 234 |
| 1539 | 1863706 | IB:1925055.5 | 1 | 155 |
| 1540 | 3950806 | IB:1925055.5 | 1 | 179 |
| 1541 | 1511496 | IB:1925055.5 | 1 | 197 |
| 1542 | 3871292 | IB:1925055.5 | 1 | 270 |
| 1543 | 2350519 | IB:1925055.5 | 1 | 200 |
| 1544 | 1500749 | IB:1925055.5 | 1 | 183 |
| 1545 | 1430632 | IB:1925055.5 | 1 | 223 |
| 1546 | 2532363 | IB:1925055.5 | 1 | 243 |
| 1547 | 629781 | IB:1925055.5 | 1 | 257 |
| 1548 | 1927213 | IB:1925055.5 | 1 | 204 |
| 1549 | 1494133 | IB:1925055.5 | 1 | 211 |
| 1550 | 2664524 | IB:1925055.5 | 1 | 234 |
| 1551 | 2635976 | IB:1925055.5 | 1 | 254 |
| 1552 | 825794 | IB:1925055.5 | 1 | 84 |
| 1553 | 2708985 | IB:1925055.5 | 1 | 297 |
| 1554 | 2538760 | IB:1925055.5 | 1 | 215 |
| 1555 | 1491886 | IB:1925055.5 | 1 | 235 |
| 1556 | 575237 | IB:1925055.5 | 1 | 253 |
| 1557 | 1909488 | IB:1925055.5 | 1 | 247 |
| 1558 | 1470258 | IB:1925055.5 | 1 | 181 |
| 1559 | 980214 | IB:1925055.5 | 1 | 331 |
| 1560 | 783555 | IB:1925055.5 | 1 | 284 |
| 1561 | 2846664 | IB:1925055.5 | 1 | 285 |
| 1562 | 2587840 | IB:1925055.5 | 1 | 248 |
| 1563 | 1417356 | IB:1925055.5 | 1 | 224 |
| 1564 | 944899 | IB:1925055.5 | 1 | 298 |
| 1565 | 588444 | IB:1925055.5 | 1 | 221 |
| 1566 | 3043960 | IB:1925055.5 | 1 | 301 |
| 1567 | 3093611 | IB:1925055.5 | 1 | 290 |
| 1568 | 1259091 | IB:1925055.5 | 1 | 224 |
| 1569 | 2847230 | IB:1925055.5 | 1 | 271 |
| 1570 | 2540514 | IB:1925055.5 | 1 | 253 |
| 1571 | 3441655 | IB:1925055.5 | 1 | 219 |
| 1572 | 2968152 | IB:1925055.5 | 1 | 316 |
| 1573 | 3398972 | IB:1925055.5 | 1 | 257 |
| 1574 | 1307709 | IB:1925055.5 | 1 | 225 |
| 1575 | 699870 | IB:1925055.5 | 1 | 225 |
| 1576 | 426967 | IB:1925055.5 | 1 | 232 |
| 1577 | 2685492 | IB:1925055.5 | 1 | 266 |
| 1578 | 3085872 | IB:1925055.5 | 2 | 95 |
| 1579 | 1391873 | IB:1925055.5 | 1 | 252 |
| 1580 | 2050275 | IB:1925055.5 | 1 | 268 |
| 1581 | 1599482 | IB:1925055.5 | 1 | 195 |
| 1582 | 851914 | IB:1925055.5 | 1 | 199 |
| 1583 | 2899326 | IB:1925055.5 | 1 | 309 |
| 1584 | 1720956 | IB:1925055.5 | 1 | 212 |
| 1585 | 1251167 | IB:1925055.5 | 1 | 250 |
| 1586 | 2968428 | IB:1925055.5 | 1 | 280 |
| 1587 | 2800404 | IB:1925055.5 | 1 | 97 |
| 1588 | 1724869 | IB:1925055.5 | 1 | 229 |
| 1589 | 2838913 | IB:1925055.5 | 1 | 261 |
| 1590 | 829618 | IB:1925055.5 | 1 | 201 |
| 1591 | 2219374 | IB:1925055.5 | 1 | 240 |
| 1592 | 2843830 | IB:1925055.5 | 1 | 265 |
| 1593 | 630799 | IB:1925055.5 | 1 | 242 |
| 1594 | 880514 | IB:1925055.5 | 1 | 251 |
| 1595 | 1581022 | IB:1925055.5 | 1 | 177 |
| 1596 | 3094485 | IB:1925055.5 | 1 | 267 |
| 1597 | 2749513 | IB:1925055.5 | 1 | 233 |
| 1598 | 3441811 | IB:1925055.5 | 1 | 228 |
| 1599 | 1303826 | IB:1925055.5 | 1 | 249 |
| 1600 | 1343609 | IB:1925055.5 | 1 | 238 |
| 1601 | 2782204 | IB:1925055.5 | 1 | 247 |
| 1602 | 782307 | IB:1925055.5 | 1 | 251 |
| 1603 | 2294574 | IB:1925055.5 | 1 | 264 |
| 1604 | 3439540 | IB:1925055.5 | 2 | 238 |
| 1605 | 1991854 | IB:1925055.5 | 1 | 212 |
| 1606 | 2683006 | IB:1925055.5 | 2 | 292 |
| 1607 | 2747201 | IB:1925055.5 | 1 | 148 |
| 1608 | 2405011 | IB:1925055.5 | 1 | 215 |
| 1609 | 1500748 | IB:1925055.5 | 1 | 198 |
| 1610 | 81829 | IB:1925055.5 | 1 | 183 |
| 1611 | 2059058 | IB:1925055.5 | 1 | 263 |
| 1612 | 3365287 | IB:1925055.5 | 1 | 151 |
| 1613 | 2841918 | IB:1925055.5 | 1 | 287 |
| 1614 | 2269574 | IB:1925055.5 | 1 | 255 |
| 1615 | 1442809 | IB:1925055.5 | 1 | 99 |
| 1616 | 2303642 | IB:1925055.5 | 1 | 249 |
| 1617 | 1704346 | IB:1925055.5 | 1 | 232 |
| 1618 | 700454 | IB:1925055.5 | 1 | 276 |
| 1619 | 1986414 | IB:1925055.5 | 1 | 289 |
| 1620 | 2614735 | IB:1925055.5 | 1 | 252 |
| 1621 | 589828 | IB:1925055.5 | 1 | 267 |
| 1622 | 2899169 | IB:1925055.5 | 1 | 287 |
| 1623 | 850494 | IB:1925055.5 | 1 | 257 |
| 1624 | 2683822 | IB:1925055.5 | 1 | 247 |
| 1625 | 2887324 | IB:1925055.5 | 1 | 125 |
| 1626 | 1282535 | IB:1925055.5 | 1 | 147 |
| 1627 | 1360106 | IB:1925055.5 | 1 | 231 |
| 1628 | 2785553 | IB:1925055.5 | 1 | 265 |
| 1629 | 1842667 | IB:1925055.5 | 1 | 291 |
| 1630 | 435086 | IB:1925055.5 | 1 | 222 |
| 1631 | 1331643 | IB:1925055.5 | 1 | 147 |
| 1632 | 2587870 | IB:1925055.5 | 1 | 214 |
| 1633 | 3043852 | IB:1925055.5 | 1 | 287 |
| 1634 | 1928212 | IB:1925055.5 | 1 | 291 |
| 1635 | 2538317 | IB:1925055.5 | 1 | 199 |
| 1636 | 1492720 | IB:1925055.5 | 1 | 212 |
| 1637 | 1361335 | IB:1925055.5 | 1 | 259 |
| 1638 | 2547050 | IB:1925055.5 | 1 | 254 |
| 1639 | 3043641 | IB:1925055.5 | 1 | 263 |
| 1640 | 1259955 | IB:1925055.5 | 1 | 125 |
| 1641 | 946266 | IB:1925055.5 | 3 | 193 |
| 1642 | 3012448 | IB:1925055.5 | 1 | 292 |
| 1643 | 420025 | IB:1925055.5 | 1 | 204 |
| 1644 | 982741 | IB:1925055.5 | 1 | 272 |
| 1645 | 2509023 | IB:1925055.5 | 1 | 254 |
| 1646 | 2670392 | IB:1925055.5 | 1 | 238 |
| 1647 | 2735656 | IB:1925055.5 | 1 | 96 |
| 1648 | 2885490 | IB:1925055.5 | 1 | 264 |
| 1649 | 1303890 | IB:1925055.5 | 1 | 231 |
| 1650 | 872731 | IB:1925055.5 | 1 | 201 |
| 1651 | 696718 | IB:1925055.5 | 1 | 232 |
| 1652 | 3688517 | IB:1925055.5 | 1 | 288 |
| 1653 | 701093781 | ZS:165234.3 | 1 | 256 |
| 1654 | 701490564 | ZS:165234.3 | 1 | 224 |
| 1655 | 701599183 | ZS:165234.3 | 1 | 260 |
| 1656 | 701616219 | ZS:165234.3 | 1 | 274 |
| 1657 | 700180910 | ZS:165234.3 | 1 | 237 |
| 1658 | 701230439 | ZS:165234.3 | 1 | 221 |
| 1659 | 701565987 | ZS:165234.3 | 1 | 298 |
| 1660 | 701469043 | ZS:165234.3 | 1 | 226 |
| 1661 | 700813572 | ZS:165234.3 | 2 | 202 |
| 1662 | 701624629 | ZS:165234.3 | 3 | 285 |
| 1663 | 701320686 | ZS:165234.3 | 43 | 175 |
| 1664 | 701506292 | ZS:165234.3 | 15 | 274 |
| 1665 | 700361285 | ZS:165234.3 | 1 | 269 |
| 1666 | 700182881 | ZS:165234.3 | 1 | 238 |
| 1667 | 700629418 | ZS:165234.3 | 1 | 262 |
| 1668 | 701468416 | ZS:165234.3 | 1 | 308 |
| 1669 | 701513663 | ZS:165234.3 | 3 | 200 |
| 1670 | 701605944 | ZS:165234.3 | 11 | 288 |
| 1671 | 701491741 | ZS:165234.3 | 1 | 212 |
| 1672 | 700539949 | ZS:165234.3 | 1 | 119 |
| 1673 | 701230808 | ZS:165234.3 | 1 | 254 |
| 1674 | 700272961 | ZS:165234.3 | 1 | 285 |
| 1675 | 701300213 | ZS:165234.3 | 6 | 199 |
| 1676 | 700628557 | ZS:165234.3 | 1 | 206 |
| 1677 | 701318893 | ZS:165234.3 | 1 | 252 |
| 1678 | 700136650 | ZS:165234.3 | 1 | 261 |
| 1679 | 701605366 | ZS:165234.3 | 1 | 273 |
| 1680 | 701344948 | ZS:165234.3 | 5 | 177 |
| 1681 | 701617705 | ZS:165234.3 | 1 | 276 |
| 1682 | 700303285 | ZS:165234.3 | 1 | 276 |
| 1683 | 701445318 | ZS:165234.3 | 1 | 223 |
| 1684 | 701514224 | ZS:165234.3 | 1 | 267 |
| 1685 | 700148731 | ZS:165234.3 | 1 | 181 |
| 1686 | 701465349 | ZS:165234.3 | 1 | 199 |
| 1687 | 701517634 | ZS:165234.3 | 1 | 217 |
| 1689 | 700504333 | ZS:165234.3 | 1 | 184 |
| 1690 | 701897857 | ZS:165234.3 | 1 | 272 |
| 1691 | 701469758 | ZS:165234.3 | 1 | 259 |
| 1693 | 700628292 | ZS:165234.3 | 1 | 237 |
| 1694 | 701582451 | ZS:165234.3 | 1 | 166 |
| 1695 | 701193139 | ZS:165234.3 | 1 | 231 |
| 1696 | 701417436 | ZS:165234.3 | 1 | 245 |
| 1697 | 701193977 | ZS:165234.3 | 1 | 248 |
| 1698 | 700609902 | ZS:165234.3 | 4 | 179 |
| 1699 | 700361875 | ZS:165234.3 | 1 | 280 |
| 1700 | 701246479 | ZS:165234.3 | 1 | 201 |
| 1701 | 701709889 | ZS:165234.3 | 1 | 269 |
| 1702 | 700503636 | ZS:165234.3 | 1 | 279 |
| 1703 | 701467565 | ZS:165234.3 | 1 | 266 |
| 1704 | 700913464 | ZS:165234.3 | 1 | 264 |
| 1705 | 701511270 | ZS:165234.3 | 1 | 263 |
| 1706 | 701510462 | ZS:165234.3 | 4 | 222 |
| 1707 | 701485744 | ZS:165234.3 | 1 | 277 |
| 1708 | 701510883 | ZS:165234.3 | 1 | 274 |
| 1709 | 700629420 | ZS:165234.3 | 1 | 263 |
| 1710 | 701474055 | ZS:165234.3 | 1 | 263 |
| 1711 | 701465268 | ZS:165234.3 | 30 | 224 |
| 1712 | 700297261 | ZS:165234.3 | 1 | 203 |
| 1713 | 700624973 | ZS:165234.3 | 1 | 269 |
| 1714 | 701466020 | ZS:165234.3 | 1 | 266 |
| 1715 | 700931834 | ZS:165234.3 | 1 | 235 |
| 1716 | 701466523 | ZS:165234.3 | 1 | 286 |
| 1717 | 701470845 | ZS:165234.3 | 2 | 166 |
| 1718 | 701490988 | ZS:165234.3 | 1 | 211 |
| 1719 | 700769675 | ZS:165234.3 | 1 | 262 |
| 1721 | 701487591 | ZS:165234.3 | 1 | 254 |
| 1722 | 700821620 | ZS:165234.3 | 1 | 234 |
| 1724 | 701560653 | ZS:165234.3 | 1 | 278 |
| 1725 | 701512645 | ZS:165234.3 | 1 | 280 |
| 1726 | 700769805 | ZS:165234.3 | 1 | 278 |
| 1727 | 701514225 | ZS:165234.3 | 3 | 274 |
| 1728 | 700290655 | ZS:165234.3 | 6 | 260 |
| 1729 | 700628888 | ZS:165234.3 | 1 | 259 |
| 1730 | 701615842 | ZS:165234.3 | 1 | 350 |
| 1731 | 700821482 | ZS:165234.3 | 1 | 239 |
| 1732 | 700143947 | ZS:165234.3 | 1 | 236 |
| 1733 | 701492871 | ZS:165234.3 | 1 | 204 |
| 1734 | 701466168 | ZS:165234.3 | 1 | 218 |
| 1735 | 701243343 | ZS:165234.3 | 1 | 160 |
| 1736 | 701491093 | ZS:165234.3 | 1 | 271 |
| 1737 | 701560741 | ZS:165234.3 | 1 | 294 |
| 1738 | 700640940 | ZS:165234.3 | 1 | 291 |
| 1739 | 701649230 | ZS:165234.3 | 4 | 323 |
| 1740 | 701940939 | ZS:165234.3 | 1 | 144 |
| 1741 | 701393081 | ZS:165234.3 | 1 | 247 |
| 1742 | 701397339 | ZS:165234.3 | 1 | 220 |
| 1743 | 700502073 | ZS:165234.3 | 1 | 202 |
| 1744 | 700934938 | ZS:165234.3 | 1 | 239 |
| 1745 | 701714541 | ZS:165234.3 | 1 | 293 |
| 1746 | 701343424 | ZS:165234.3 | 1 | 241 |
| 1747 | 700229933 | ZS:165234.3 | 1 | 287 |
| 1748 | 701093696 | ZS:165234.3 | 1 | 251 |
| 1749 | 701610104 | ZS:165234.3 | 1 | 333 |
| 1750 | 700288991 | ZS:165234.3 | 1 | 286 |
| 1751 | 700136250 | ZS:165234.3 | 1 | 254 |
| 1752 | 1371843 | IB:2094678.8 | 1 | 237 |
| 1753 | 2688148 | IB:2094678.9 | 1 | 254 |
| 1754 | 2875106 | IB:2094678.9 | 1 | 278 |
| 1755 | 587415 | IB:2094678.9 | 7 | 100 |
| 1756 | 700190695 | ZS:164307.1 | 1 | 277 |
| 1757 | 701283151 | ZS:164307.2 | 1 | 146 |
| 1758 | 700228775 | ZS:164307.2 | 1 | 121 |
| 1759 | 700417976 | ZS:164307.2 | 1 | 229 |
| 1760 | 700179881 | ZS:164307.2 | 1 | 193 |
| 1761 | 701023034 | ZS:164307.2 | 1 | 215 |
| 1762 | 700524975 | ZS:164307.2 | 1 | 277 |
| 1763 | 700268791 | ZS:164307.2 | 1 | 253 |
| 1764 | 700066924 | ZS:164307.2 | 1 | 207 |
| 1765 | 700294785 | ZS:164307.2 | 1 | 223 |
| 1766 | 700478479 | ZS:164307.2 | 1 | 229 |
| 1767 | 700059848 | ZS:164307.2 | 1 | 194 |
| 1768 | 700627741 | ZS:164307.3 | 1 | 232 |
| 1769 | 700627895 | ZS:164307.3 | 1 | 231 |
| 1771 | 700498943 | ZS:164307.3 | 6 | 94 |
| 1772 | 700280214 | ZS:164307.3 | 1 | 84 |
| 1773 | 700501772 | ZS:164307.3 | 1 | 271 |
| 1774 | 700177732 | ZS:164307.3 | 1 | 195 |
| 1775 | 701881119 | ZS:164307.3 | 1 | 293 |
| 1776 | 700533367 | ZS:164307.3 | 1 | 147 |
| 1777 | 701294328 | ZS:164307.3 | 1 | 246 |
| 1778 | 700364549 | ZS:164307.3 | 1 | 96 |
| 1779 | 700307292 | ZS:164307.3 | 1 | 280 |
| 1780 | 701475321 | ZS:164307.3 | 1 | 207 |
| 1781 | 701944916 | ZS:164307.3 | 1 | 240 |
| 1782 | 701285043 | ZS:164307.3 | 1 | 245 |
| 1784 | 700628168 | ZS:164307.3 | 1 | 219 |
| 1785 | 701192218 | ZS:164307.3 | 1 | 117 |
| 1786 | 700534239 | ZS:164307.3 | 1 | 277 |
| 1787 | 700326287 | ZS:164307.3 | 3 | 256 |
| 1788 | 4448044 | LG:2022180.2 | 1 | 274 |
| 1789 | 6538054 | LG:2022180.6 | 1 | 563 |
| 1790 | 5660005 | LG:2022180.6 | 1 | 262 |
| 1791 | 6510029 | LG:2022180.7 | 1 | 572 |
| 1792 | 6480568 | LG:2022180.7 | 1 | 508 |
| 1793 | 5812622 | LG:2022180.7 | 1 | 292 |
| 1794 | 6354669 | LG:2022180.7 | 1 | 192 |
| 1795 | 4443286 | LG:2022180.7 | 1 | 248 |
| 1796 | 6060942 | LG:2022180.7 | 1 | 297 |
| 1797 | 6093338 | LG:2022180.7 | 1 | 284 |
| 1798 | 5943867 | LG:2022180.7 | 1 | 277 |
| 1799 | 6312930 | LG:2022180.7 | 1 | 374 |
| 1800 | 6127343 | LG:2022180.7 | 1 | 407 |
| 1801 | 6131166 | LG:2022180.7 | 1 | 322 |
| 1802 | 6059741 | LG:2022180.7 | 1 | 94 |
| 1803 | 6012595 | LG:2022180.7 | 1 | 281 |
| 1804 | 5946232 | LG:2022180.7 | 1 | 285 |
| 1805 | 6350367 | LG:2022180.7 | 1 | 297 |
| 1806 | 6013556 | LG:2022180.7 | 1 | 268 |
| 1807 | 6383511 | LG:2022180.7 | 1 | 156 |
| 1808 | 6121245 | LG:2022180.7 | 1 | 499 |
| 1809 | 6566762 | LG:2022180.7 | 1 | 534 |
| 1810 | 6350903 | LG:2022180.7 | 1 | 300 |
| 1811 | 6192825 | LG:2022180.7 | 1 | 312 |
| 1812 | 6383743 | LG:2022180.7 | 1 | 88 |
| 1813 | 5952122 | LG:2022180.7 | 1 | 333 |
| 1814 | 5992338 | LG:2022180.7 | 1 | 300 |
| 1815 | 5876091 | LG:2022180.7 | 1 | 282 |
| 1816 | 5887762 | LG:2022180.7 | 1 | 265 |
| 1817 | 6264693 | LG:2022180.7 | 1 | 572 |
| 1818 | 1894667 | LG:2022180.7 | 1 | 248 |
| 1819 | 6298742 | LG:2022180.7 | 8 | 274 |
| 1820 | 5887801 | LG:2022180.7 | 1 | 273 |
| 1821 | 6370049 | LG:2022180.7 | 1 | 436 |
| 1822 | 6498016 | LG:2022180.7 | 1 | 335 |
| 1823 | 6213424 | LG:2022180.7 | 1 | 292 |
| 1824 | 5844137 | LG:2022180.7 | 1 | 254 |
| 1825 | 6424826 | LG:2022180.7 | 1 | 476 |
| 1826 | 6058776 | LG:2022180.7 | 3 | 377 |
| 1827 | 5839201 | LG:2022180.7 | 1 | 95 |
| 1828 | 6510129 | LG:2022180.7 | 1 | 507 |
| 1829 | 3105267 | IB:2071401.2 | 1 | 294 |
| 1830 | 700062910 | ZS:159749.1 | 2 | 303 |
| 1831 | 700067568 | ZS:159749.1 | 1 | 268 |
| 1832 | 700308813 | ZS:159749.1 | 1 | 100 |
| 1833 | 700180966 | ZS:159749.1 | 1 | 264 |
| 1834 | 700510020 | ZS:159749.2 | 1 | 245 |
| 1835 | 701335324 | ZS:159749.2 | 1 | 204 |
| 1836 | 700918201 | ZS:159749.2 | 1 | 88 |
| 1837 | 701286045 | ZS:159749.2 | 1 | 254 |
| 1838 | 700290714 | ZS:170990.1 | 1 | 290 |
| 1839 | 700190967 | ZS:170990.1 | 1 | 150 |
| 1840 | 700302994 | ZS:170990.1 | 1 | 289 |
| 1841 | 2010193 | IB:2097646.3 | 1 | 80 |
| 1842 | 2823003 | IB:2097646.3 | 1 | 244 |
| 1843 | 59307 | IB:2097646.3 | 1 | 144 |
| 1844 | 2820251 | IB:2097646.3 | 2 | 231 |
| 1845 | 1240147 | IB:2097646.3 | 1 | 200 |
| 1846 | 82699 | IB:2097646.3 | 1 | 145 |
| 1847 | 2825725 | IB:2097646.3 | 3 | 167 |
| 1848 | 442 | IB:2097646.3 | 13 | 497 |
| 1849 | 725067 | IB:2097646.3 | 1 | 106 |
| 1850 | 82700 | IB:2097646.3 | 1 | 188 |
| 1851 | 364676 | IB:2097646.3 | 11 | 383 |
| 1852 | 1686 | IB:2097646.3 | 1 | 264 |
| 1853 | 78129 | IB:2097646.3 | 1 | 276 |
| 1854 | 2971626 | IB:2097646.3 | 1 | 55 |
| 1855 | 189009 | IB:2097646.3 | 1 | 241 |
| 1856 | 149464 | IB:2097646.3 | 1 | 211 |
| 1857 | 568750 | IB:2097646.3 | 3 | 268 |
| 1858 | 1103 | IB:2097646.3 | 1 | 471 |
| 1859 | 993 | IB:2097646.3 | 1 | 329 |
| 1860 | 36161 | IB:2097646.3 | 1 | 197 |
| 1861 | 42068 | IB:2097646.3 | 1 | 226 |
| 1862 | 46272 | IB:2097646.3 | 1 | 223 |
| 1863 | 1471176 | IB:652960.1 | 1 | 223 |
| 1864 | 911228 | IB:2094701.10 | 1 | 258 |
| 1865 | 168804 | IB:2094701.10 | 11 | 305 |
| 1866 | 900839 | IB:2094701.10 | 3 | 125 |
| 1867 | 3897459 | IB:2094701.10 | 1 | 256 |
| 1868 | 1840483 | IB:2094701.10 | 1 | 253 |
| 1869 | 899181 | IB:2094701.10 | 1 | 246 |
| 1870 | 701024520 | ZS:157944.4 | 1 | 275 |
| 1871 | 700420532 | ZS:157944.4 | 1 | 180 |
| 1872 | 700930974 | ZS:157944.4 | 3 | 252 |
| 1873 | 700607265 | ZS:157944.4 | 1 | 282 |
| 1874 | 701246834 | ZS:157944.4 | 1 | 245 |
| 1875 | 700609603 | ZS:157944.4 | 1 | 289 |
| 1876 | 701396879 | ZS:157944.4 | 1 | 182 |
| 1877 | 701480622 | ZS:157944.4 | 2 | 262 |
| 1878 | 701343245 | ZS:157944.4 | 1 | 234 |
| 1879 | 6554941 | LG:2020640.7 | 1 | 601 |
| 1880 | 6559814 | LG:2020640.7 | 1 | 559 |
| 1881 | 700483216 | ZS:159079.1 | 1 | 255 |
| 1882 | 700483123 | ZS:159079.1 | 1 | 269 |
| 1883 | 700780819 | ZS:159079.2 | 1 | 228 |
| 1884 | 701485977 | ZS:159079.2 | 1 | 262 |
| 1885 | 701318240 | ZS:159079.2 | 1 | 192 |
| 1886 | 701258865 | ZS:159079.2 | 1 | 241 |
| 1887 | 700314533 | ZS:159079.2 | 1 | 209 |
| 1888 | 700435941 | ZS:159079.2 | 2 | 161 |
| 1889 | 700313454 | ZS:159079.2 | 1 | 253 |
| 1890 | 700938719 | ZS:159079.2 | 1 | 112 |
| 1891 | 700420843 | ZS:159079.2 | 1 | 216 |
| 1892 | 701275345 | ZS:159079.2 | 1 | 268 |
| 1893 | 700778749 | ZS:159079.2 | 1 | 92 |
| 1894 | 701394518 | ZS:159079.2 | 1 | 246 |
| 1895 | 701879481 | ZS:159079.2 | 1 | 212 |
| 1896 | 700939406 | ZS:159079.2 | 1 | 263 |
| 1897 | 701909492 | ZS:159079.2 | 1 | 269 |
| 1898 | 700780985 | ZS:159079.2 | 1 | 251 |
| 1899 | 701318033 | ZS:159079.2 | 1 | 185 |
| 1900 | 700525373 | ZS:159079.2 | 1 | 105 |
| 1901 | 5920416 | LG:2020640.6 | 1 | 286 |
| 1902 | 5885823 | LG:2020640.6 | 1 | 252 |
| 1903 | 5881319 | LG:2020640.6 | 1 | 159 |
| 1904 | 6270301 | LG:2020640.6 | 1 | 91 |
| 1905 | 6271586 | LG:2020640.6 | 12 | 493 |
| 1906 | 6552641 | LG:2020640.6 | 1 | 478 |
| 1907 | 5891134 | LG:2020640.6 | 1 | 268 |
| 1908 | 6116862 | LG:2020640.9 | 1 | 284 |
| 1909 | 6424889 | LG:2020640.9 | 1 | 578 |
| 1910 | 5616328 | LG:2020640.9 | 2 | 280 |
| 1911 | 5990859 | LG:2020640.9 | 1 | 305 |
| 1912 | 6598719 | LG:2020640.9 | 1 | 536 |
| 1913 | 6379254 | LG:2020640.9 | 1 | 278 |
| 1914 | 3760758 | LG:2020640.9 | 1 | 306 |
| 1915 | 5839547 | LG:2020640.9 | 2 | 217 |
| 1916 | 4515874 | LG:2020640.9 | 1 | 203 |
| 1917 | 5476167 | LG:2020640.9 | 1 | 246 |
| 1918 | 6424790 | LG:2020640.9 | 1 | 482 |
| 1919 | 6370928 | LG:2020640.9 | 1 | 221 |
| 1920 | 6178092 | LG:2020640.9 | 1 | 281 |
| 1921 | 5475523 | LG:2020640.9 | 1 | 254 |
| 1922 | 6496850 | LG:2020640.9 | 1 | 575 |
| 1923 | 5894616 | LG:2020640.9 | 1 | 250 |
| 1924 | 5474001 | LG:2020640.9 | 1 | 255 |
| 1925 | 6065772 | LG:2020640.9 | 1 | 283 |
| 1926 | 6152091 | LG:2020640.9 | 1 | 273 |
| 1927 | 6125646 | LG:2020640.9 | 1 | 517 |
| 1928 | 6264310 | LG:2020640.9 | 1 | 562 |
| 1929 | 6538510 | LG:2020640.9 | 1 | 569 |
| 1930 | 6262111 | LG:2020640.9 | 1 | 546 |
| 1931 | 6190138 | LG:2020640.9 | 1 | 290 |
| 1932 | 5941339 | LG:1900605.9 | 1 | 269 |
| 1933 | 3903719 | LG:1900605.9 | 1 | 285 |
| 1934 | 6026458 | LG:1900605.6 | 1 | 302 |
| 1935 | 6113765 | LG:1981103.6 | 1 | 292 |
| 1936 | 6262544 | LG:1981103.6 | 1 | 461 |
| 1937 | 6425437 | LG:1981103.6 | 2 | 534 |
| 1938 | 6211146 | LG:1981103.6 | 1 | 223 |
| 1939 | 6114029 | LG:1981103.6 | 1 | 274 |
| 1940 | 6553732 | LG:1981103.6 | 1 | 417 |
| 1941 | 6323421 | LG:1981103.6 | 1 | 287 |
| 1942 | 6296822 | LG:1981103.6 | 1 | 335 |
| 1943 | 6606417 | LG:1981103.6 | 1 | 111 |
| 1944 | 6496467 | LG:1981103.6 | 1 | 511 |
| 1945 | 6518170 | LG:1981103.6 | 3 | 65 |
| 1946 | 6353795 | LG:1981103.6 | 1 | 179 |
| 1947 | 6604422 | LG:1981103.6 | 1 | 523 |
| 1948 | 6386936 | LG:1981103.6 | 1 | 297 |
| 1949 | 6130913 | LG:1981103.6 | 3 | 262 |
| 1950 | 4519218 | LG:1981103.6 | 1 | 247 |
| 1951 | 6435737 | LG:1981103.6 | 1 | 325 |
| 1952 | 5475918 | LG:1981103.6 | 1 | 253 |
| 1953 | 6542807 | LG:1981103.6 | 1 | 532 |
| 1954 | 6317860 | LG:1981103.6 | 1 | 302 |
| 1955 | 3023731 | LG:1981103.6 | 1 | 277 |
| 1956 | 6484989 | LG:1981103.5 | 1 | 457 |
| 1957 | 1467992 | IB:2094696.1 | 1 | 206 |
| 1958 | 908408 | IB:2094701.6 | 1 | 66 |
| 1959 | 487818 | IB:2094701.8 | 1 | 249 |
| 1960 | 490900 | IB:2094701.8 | 1 | 268 |
| 1961 | 700273719 | ZS:053563.1 | 1 | 254 |
| 1962 | 3444223 | LG:2024986.3 | 1 | 141 |
| 1963 | 5989011 | LG:2024986.3 | 1 | 292 |
| 1964 | 5872059 | LG:2024986.3 | 1 | 296 |
| 1965 | 6095094 | LG:2024986.3 | 1 | 318 |
| 1966 | 6093023 | LG:2024986.3 | 1 | 298 |
| 1967 | 6075596 | LG:2024986.3 | 1 | 306 |
| 1968 | 6419675 | LG:2024986.3 | 1 | 510 |
| 1969 | 6209547 | LG:2024986.3 | 1 | 294 |
| 1970 | 6405076 | LG:2024986.3 | 3 | 205 |
| 1971 | 6078425 | LG:2024986.3 | 3 | 282 |
| 1972 | 5981860 | LG:2024986.3 | 1 | 278 |
| 1973 | 5983358 | LG:2024986.3 | 1 | 281 |
| 1974 | 5979930 | LG:2024986.3 | 1 | 272 |
| 1975 | 6261877 | LG:2024986.3 | 1 | 282 |
| 1976 | 6375405 | LG:2024986.3 | 2 | 273 |
| 1977 | 5981662 | LG:2024986.3 | 3 | 272 |
| 1978 | 6564362 | LG:2024986.3 | 1 | 532 |
| 1979 | 6422216 | LG:2024986.3 | 1 | 497 |
| 1980 | 3902250 | LG:2024986.3 | 1 | 274 |
| 1981 | 5953976 | LG:2024986.3 | 1 | 322 |
| 1982 | 6133582 | LG:2024986.3 | 9 | 281 |
| 1983 | 3166285 | LG:2024986.3 | 3 | 244 |
| 1984 | 6154521 | LG:2024986.3 | 7 | 326 |
| 1985 | 5564143 | LG:2024986.3 | 1 | 261 |
| 1986 | 6131318 | LG:2024986.3 | 6 | 223 |
| 1987 | 6114440 | LG:2024986.3 | 2 | 267 |
| 1988 | 6564795 | LG:2024986.3 | 1 | 615 |
| 1989 | 6131579 | LG:2024986.3 | 1 | 296 |
| 1990 | 6299814 | LG:2024986.3 | 2 | 287 |
| 1991 | 6151928 | LG:2024986.3 | 3 | 245 |
| 1992 | 6388403 | LG:2024986.3 | 1 | 327 |
| 1993 | 6150440 | LG:2024986.3 | 3 | 275 |
| 1994 | 3165833 | LG:2024986.3 | 3 | 269 |
| 1995 | 6396535 | LG:2024986.3 | 1 | 285 |
| 1996 | 6361347 | LG:2024986.3 | 1 | 305 |
| 1997 | 5984828 | LG:2024986.3 | 1 | 284 |
| 1998 | 5987020 | LG:2024986.3 | 1 | 282 |
| 1999 | 6408470 | LG:2024986.3 | 9 | 545 |
| 2000 | 6270190 | LG:2024986.3 | 1 | 522 |
| 2001 | 5983366 | LG:2024986.3 | 1 | 293 |
| 2002 | 6422308 | LG:2024986.3 | 1 | 509 |
| 2003 | 5908769 | LG:2024986.3 | 2 | 295 |
| 2004 | 5658363 | LG:2024986.3 | 1 | 263 |
| 2005 | 6454559 | LG:2024986.3 | 1 | 596 |
| 2006 | 5475468 | LG:2024986.3 | 1 | 276 |
| 2007 | 6420636 | LG:2024986.3 | 1 | 493 |
| 2008 | 6538777 | LG:2024986.3 | 1 | 147 |
| 2009 | 3444239 | LG:2024986.3 | 1 | 263 |
| 2010 | 1896359 | LG:2024986.3 | 1 | 246 |
| 2011 | 6112991 | LG:2024986.3 | 2 | 273 |
| 2012 | 5986966 | LG:2024986.3 | 1 | 271 |
| 2013 | 5809528 | LG:2024986.3 | 1 | 286 |
| 2014 | 5987770 | LG:2024986.3 | 1 | 282 |
| 2015 | 5988150 | LG:2024986.3 | 1 | 269 |
| 2016 | 6561673 | LG:2024986.3 | 1 | 570 |
| 2017 | 6425449 | LG:2024986.3 | 1 | 139 |
| 2018 | 6422516 | LG:2024986.3 | 1 | 79 |
| 2019 | 4516438 | LG:2024986.3 | 1 | 259 |
| 2020 | 6514835 | LG:2024986.3 | 1 | 580 |
| 2021 | 1895668 | LG:2024986.3 | 1 | 250 |
| 2022 | 6170203 | LG:2024986.3 | 1 | 286 |
| 2023 | 6402652 | LG:2024986.3 | 1 | 288 |
| 2024 | 6095029 | LG:2024986.3 | 1 | 331 |
| 2025 | 5879126 | LG:2024986.3 | 2 | 264 |
| 2026 | 4123126 | LG:2024986.3 | 2 | 90 |
| 2027 | 5563507 | LG:2024986.3 | 1 | 246 |
| 2028 | 6123211 | LG:2024986.3 | 1 | 571 |
| 2029 | 6371816 | LG:2024986.3 | 1 | 172 |
| 2030 | 3158120 | LG:2024986.3 | 2 | 158 |
| 2031 | 6402620 | LG:2024986.3 | 1 | 269 |
| 2032 | 5954076 | LG:2024986.3 | 1 | 266 |
| 2033 | 5870789 | LG:2024986.3 | 1 | 281 |
| 2034 | 6419582 | LG:2024986.3 | 1 | 517 |
| 2035 | 6497557 | LG:2024986.3 | 1 | 184 |
| 2036 | 700269380 | ZS:177283.1 | 1 | 309 |
| 2037 | 700061367 | ZS:177283.2 | 3 | 95 |
| 2038 | 700484428 | ZS:177283.2 | 1 | 255 |
| 2039 | 6512973 | LG:1941236.1 | 1 | 548 |
| 2040 | 30396 | IB:1900605.10 | 2 | 226 |
| 2041 | 2113 | IB:1900605.10 | 1 | 443 |
| 2042 | 2741933 | IB:1900605.10 | 1 | 237 |
| 2043 | 100690 | IB:1900605.10 | 1 | 227 |
| 2044 | 2304342 | IB:1900605.10 | 1 | 270 |
| 2045 | 1807710 | IB:1900605.6 | 1 | 202 |
| 2046 | 1295954 | IB:1900605.6 | 1 | 239 |
| 2047 | 52759 | IB:1900605.8 | 1 | 234 |
| 2048 | 2797366 | IB:1900605.8 | 3 | 245 |
| 2049 | 2906493 | IB:1900605.8 | 1 | 277 |
| 2050 | 2729355 | IB:1900605.9 | 3 | 255 |
| 2051 | 700910295 | ZS:087251.1 | 1 | 71 |
| 2052 | 782418 | IB:2104891.5 | 2 | 277 |
| 2053 | 1430906 | IB:2104891.5 | 1 | 243 |
| 2054 | 2455564 | IB:2104891.6 | 1 | 245 |
| 2055 | 3086914 | IB:2104891.6 | 1 | 223 |
| 2056 | 1634012 | IB:2104891.7 | 1 | 215 |
| 2057 | 1216570 | IB:2104891.7 | 1 | 319 |
| 2058 | 2841268 | IB:2104891.7 | 1 | 174 |
| 2059 | 830485 | IB:2104891.8 | 1 | 249 |
| 2060 | 233757 | IB:2104891.8 | 109 | 147 |
| 2061 | 57367 | IB:2104891.8 | 1 | 210 |
| 2062 | 2194388 | IB:2104891.8 | 1 | 240 |
| 2063 | 766272 | IB:2104891.8 | 1 | 246 |
| 2064 | 750798 | IB:2104891.8 | 1 | 215 |
| 2065 | 935410 | IB:2104891.8 | 1 | 287 |
| 2066 | 825399 | IB:2104891.8 | 1 | 249 |
| 2067 | 601853 | IB:2104891.8 | 1 | 213 |
| 2068 | 2522237 | IB:2104891.8 | 1 | 254 |
| 2069 | 1333153 | IB:2104891.8 | 1 | 234 |
| 2070 | 2085612 | IB:2104891.8 | 1 | 249 |
| 2071 | 935740 | IB:2104891.8 | 1 | 278 |
| 2072 | 523577 | IB:2104891.8 | 1 | 221 |
| 2073 | 866666 | IB:2104891.8 | 23 | 75 |
| 2074 | 2951387 | IB:2104891.8 | 1 | 264 |
| 2075 | 1430663 | IB:2104891.8 | 1 | 247 |
| 2076 | 1361476 | IB:2104891.8 | 1 | 251 |
| 2077 | 1560126 | IB:2104891.8 | 1 | 218 |
| 2078 | 744459 | IB:2104891.8 | 1 | 258 |
| 2079 | 1797747 | IB:2104891.8 | 1 | 233 |
| 2080 | 2183401 | IB:2104891.8 | 1 | 271 |
| 2081 | 603570 | IB:2104891.8 | 1 | 254 |
| 2082 | 942875 | IB:2104891.8 | 1 | 231 |
| 2083 | 1457010 | IB:2104891.8 | 1 | 252 |
| 2084 | 2212106 | IB:2104891.8 | 1 | 254 |
| 2085 | 2451987 | IB:2104891.8 | 1 | 91 |
| 2086 | 1419958 | IB:2104891.8 | 23 | 237 |
| 2087 | 1903227 | IB:2104891.8 | 1 | 273 |
| 2088 | 35143 | IB:2104891.8 | 1 | 165 |
| 2089 | 1647305 | IB:2104891.8 | 1 | 197 |
| 2090 | 2057559 | IB:2104891.8 | 1 | 270 |
| 2091 | 2079008 | IB:2104891.8 | 1 | 228 |
| 2092 | 2174488 | IB:2104891.8 | 1 | 165 |
| 2093 | 1806270 | IB:2104891.8 | 1 | 279 |
| 2094 | 2472046 | IB:2104891.8 | 1 | 229 |
| 2095 | 1493214 | IB:2104891.8 | 1 | 223 |
| 2096 | 853974 | IB:2104891.8 | 1 | 238 |
| 2097 | 900309 | IB:2104891.8 | 1 | 284 |
| 2098 | 3868358 | IB:2104891.8 | 1 | 184 |
| 2099 | 2914677 | IB:2104891.8 | 1 | 280 |
| 2100 | 2322455 | IB:2104891.8 | 1 | 263 |
| 2101 | 966061 | IB:2104891.8 | 1 | 262 |
| 2102 | 1664515 | IB:2104891.8 | 1 | 240 |
| 2103 | 825238 | IB:2104891.8 | 1 | 232 |
| 2104 | 1556945 | IB:2104891.8 | 1 | 206 |
| 2105 | 2602985 | IB:2104891.8 | 1 | 283 |
| 2106 | 1680495 | IB:2104891.8 | 1 | 230 |
| 2107 | 1794004 | IB:2104891.8 | 1 | 276 |
| 2108 | 83448 | IB:2104891.8 | 1 | 151 |
| 2109 | 1267536 | IB:2104891.8 | 1 | 141 |
| 2110 | 2106813 | IB:2104891.8 | 1 | 225 |
| 2111 | 1510165 | IB:2104891.8 | 1 | 212 |
| 2112 | 1856492 | IB:2104891.8 | 1 | 275 |
| 2113 | 2266659 | IB:2104891.8 | 1 | 259 |
| 2114 | 1558625 | IB:2104891.8 | 1 | 210 |
| 2115 | 233733 | IB:2104891.8 | 209 | 249 |
| 2116 | 1551996 | IB:2104891.8 | 1 | 199 |
| 2117 | 1986820 | IB:2104891.8 | 1 | 259 |
| 2118 | 877233 | IB:2104891.8 | 1 | 323 |
| 2119 | 2256047 | IB:2104891.8 | 1 | 250 |
| 2120 | 1911310 | IB:2104891.8 | 1 | 245 |
| 2121 | 678398 | IB:2104891.8 | 3 | 300 |
| 2122 | 1804361 | IB:2104891.8 | 1 | 264 |
| 2123 | 935092 | IB:2104891.8 | 1 | 286 |
| 2124 | 1905732 | IB:2104891.8 | 1 | 265 |
| 2125 | 1614400 | IB:2104891.8 | 1 | 174 |
| 2126 | 35182 | IB:2104891.8 | 1 | 232 |
| 2127 | 3326065 | IB:2104891.8 | 1 | 231 |
| 2128 | 582185 | IB:2104891.8 | 1 | 264 |
| 2129 | 991896 | IB:2104891.8 | 1 | 270 |
| 2130 | 1212159 | IB:2104891.8 | 1 | 226 |
| 2131 | 555259 | IB:2104891.8 | 1 | 274 |
| 2132 | 798805 | IB:2104891.8 | 1 | 276 |
| 2133 | 2626808 | IB:2104891.8 | 1 | 244 |
| 2134 | 2758919 | IB:2104891.8 | 1 | 135 |
| 2135 | 1618796 | IB:2104891.8 | 1 | 208 |
| 2136 | 1640142 | IB:2104891.8 | 1 | 206 |
| 2137 | 3228228 | IB:2104891.8 | 1 | 168 |
| 2138 | 685491 | IB:2104891.8 | 1 | 189 |
| 2139 | 1443405 | IB:2104891.8 | 1 | 259 |
| 2140 | 1305226 | IB:2104891.8 | 1 | 202 |
| 2141 | 751876 | IB:2104891.8 | 1 | 228 |
| 2142 | 3289296 | IB:2104891.8 | 1 | 99 |
| 2143 | 1555366 | IB:2104891.8 | 1 | 215 |
| 2144 | 2324241 | IB:2104891.8 | 1 | 247 |
| 2145 | 3201317 | IB:2104891.8 | 1 | 95 |
| 2146 | 1379439 | IB:2104891.8 | 1 | 191 |
| 2147 | 2461925 | IB:2104891.8 | 1 | 226 |
| 2148 | 902077 | IB:2104891.8 | 1 | 264 |
| 2149 | 1482170 | IB:2104891.8 | 1 | 266 |
| 2150 | 586905 | IB:2104891.8 | 1 | 305 |
| 2151 | 2445442 | IB:2104891.8 | 1 | 232 |
| 2152 | 5850349 | LG:1900605.10 | 1 | 138 |
| 2153 | 6520711 | LG:1900605.10 | 1 | 466 |
| 2154 | 5742854 | LG:1900605.11 | 1 | 294 |
| 2155 | 6589010 | LG:1900605.11 | 1 | 447 |
| 2156 | 5679919 | LG:1900605.11 | 1 | 107 |
| 2157 | 1892562 | LG:1900605.11 | 1 | 188 |
| 2158 | 5985587 | LG:1900605.11 | 1 | 264 |
| 2159 | 3780406 | LG:1900605.11 | 2 | 316 |
| 2160 | 4205646 | LG:1900605.11 | 1 | 250 |
| 2161 | 6284143 | LG:1900605.11 | 1 | 247 |
| 2162 | 6259745 | LG:1900605.11 | 1 | 274 |
| 2163 | 6361023 | LG:1900605.11 | 1 | 522 |
| 2164 | 6517006 | LG:1900605.11 | 1 | 66 |
| 2165 | 6522491 | LG:1900605.11 | 1 | 257 |
| 2166 | 6496384 | LG:1900605.11 | 1 | 558 |
| 2167 | 5873053 | LG:1900605.11 | 1 | 274 |
| 2168 | 6360111 | LG:1900605.11 | 1 | 473 |
| 2169 | 5657335 | LG:1900605.11 | 1 | 244 |
| 2170 | 6395718 | LG:1900605.11 | 2 | 180 |
| 2171 | 6260645 | LG:1900605.11 | 1 | 177 |

TABLE 3

| SEQ ID NO | INCYTE ID N | GENBANK I | GENBANK ANNOTATION |
|---|---|---|---|
| 1 | hu01031982 | g4753757 | dJ798A17.1 (Flavin-containing Monooxygenase 1 (FMO-1) |
| 2 | hu00713471 | g5295993 | Human SDHD gene for small subunit of cytochrome b of succinate dehydrogenase, complete cds. |
| 3 | hu01170046 | g2588778 | Human mRNA for cytochrome b large subunit of complex II, complete cds. |
| 4 | hu01180073 | g2588778 | Human mRNA for cytochrome b large subunit of complex II, complete cds. |
| 5 | hu01201763 | g2588778 | Human mRNA for cytochrome b large subunit of complex II, complete cds. |
| 6 | hu01322950 | g2588778 | Human mRNA for cytochrome b large subunit of complex II, complete cds. |
| 7 | hu01330729 | g2588778 | Human mRNA for cytochrome b large subunit of complex II, complete cds. |
| 8 | hu00983306 | g2588778 | Human mRNA for cytochrome b large subunit of complex II, complete cds. |
| 9 | hu01239428 | g2588778 | Human mRNA for cytochrome b large subunit of complex II, complete cds. |
| 10 | hu01185563 | g2588778 | Human mRNA for cytochrome b large subunit of complex II, complete cds. |
| 11 | hu01313867 | g2588778 | Human mRNA for cytochrome b large subunit of complex II, complete cds. |
| 12 | hu01140406 | g2588778 | Human mRNA for cytochrome b large subunit of complex II, complete cds. |
| 13 | hu01228388 | g2588778 | Human mRNA for cytochrome b large subunit of complex II, complete cds. |
| 14 | hu00981727 | g2588778 | Human mRNA for cytochrome b large subunit of complex II, complete cds. |
| 15 | hu01186567 | g2588778 | Human mRNA for cytochrome b large subunit of complex II, complete cds. |
| 16 | hu01122732 | g2588778 | Human mRNA for cytochrome b large subunit of complex II, complete cds. |
| 17 | hu01012436 | g181260 | Human somatic cytochrome c (HC5) processed pseudogene, complete cds. |
| 18 | hu01002677 | g4200327 | thioredoxin |
| 19 | hu00978833 | g531404 | Human mRNA for glutaredoxin. |
| 20 | hu01284599 | g531404 | Human mRNA for glutaredoxin. |
| 21 | hu01086979 | g531404 | Human mRNA for glutaredoxin. |
| 22 | hu01032083 | g531404 | Human mRNA for glutaredoxin. |
| 23 | hu01210547 | g531404 | Human mRNA for glutaredoxin. |
| 24 | hu01123095 | g531404 | Human mRNA for glutaredoxin. |
| 25 | hu01329043 | g531404 | Human mRNA for glutaredoxin. |
| 26 | hu01116805 | g531404 | Human mRNA for glutaredoxin. |
| 27 | hu01102991 | g531404 | Human mRNA for glutaredoxin. |
| 28 | hu01013649 | g531404 | Human mRNA for glutaredoxin. |
| 29 | hu01243383 | g531404 | Human mRNA for glutaredoxin. |
| 30 | hu01293306 | g531404 | Human mRNA for glutaredoxin. |
| 31 | hu01104919 | g531404 | Human mRNA for glutaredoxin. |
| 32 | hu01329433 | g531404 | Human mRNA for glutaredoxin. |
| 33 | hu00986013 | g531404 | Human mRNA for glutaredoxin. |
| 34 | hu01078335 | g531404 | Human mRNA for glutaredoxin. |
| 35 | hu01034993 | g531404 | Human mRNA for glutaredoxin. |
| 36 | hu01332579 | g531404 | Human mRNA for glutaredoxin. |
| 37 | hu01267654 | g531404 | Human mRNA for glutaredoxin. |
| 38 | hu01175359 | g531404 | Human mRNA for glutaredoxin. |
| 39 | hu01193983 | g531404 | Human mRNA for glutaredoxin. |
| 40 | hu00755546 | g3877611 | similar to cytochrome B561 |
| 41 | hu00668597 | g2662290 | Human mRNA for cytochrome b5, partial cds. |
| 42 | hu00899952 | g2662290 | Human mRNA for cytochrome b5, partial cds. |
| 43 | hu00504101 | g2662290 | Human mRNA for cytochrome b5, partial cds. |
| 44 | hu00090502 | g2662290 | Human mRNA for cytochrome b5, partial cds. |
| 45 | hu00902140 | g2662290 | Human mRNA for cytochrome b5, partial cds. |
| 46 | hu00503171 | g2662290 | Human mRNA for cytochrome b5, partial cds. |
| 47 | hu00046235 | g2662290 | Human mRNA for cytochrome b5, partial cds. |
| 48 | hu00007392 | g2662290 | Human mRNA for cytochrome b5, partial cds. |
| 49 | hu00381091 | g2662290 | Human mRNA for cytochrome b5, partial cds. |
| 50 | hu00562460 | g2662290 | Human mRNA for cytochrome b5, partial cds. |
| 51 | hu00039191 | g2662290 | Human mRNA for cytochrome b5, partial cds. |
| 52 | hu00605418 | g2662290 | Human mRNA for cytochrome b5, partial cds. |
| 53 | hu00725335 | g2662290 | Human mRNA for cytochrome b5, partial cds. |
| 54 | hu00651653 | g2662290 | Human mRNA for cytochrome b5, partial cds. |
| 55 | hu00916613 | g2662290 | Human mRNA for cytochrome b5, partial cds. |
| 56 | hu00814652 | g2662290 | Human mRNA for cytochrome b5, partial cds. |
| 57 | hu00163808 | g2662290 | Human mRNA for cytochrome b5, partial cds. |
| 58 | hu00687420 | g2662290 | Human mRNA for cytochrome b5, partial cds. |
| 59 | hu00238960 | g2662290 | Human mRNA for cytochrome b5, partial cds. |
| 60 | hu00776351 | g2662290 | Human mRNA for cytochrome b5, partial cds. |
| 61 | hu00883244 | g2662290 | Human mRNA for cytochrome b5, partial cds. |
| 62 | hu00816546 | g2662290 | Human mRNA for cytochrome b5, partial cds. |
| 63 | hu00861826 | g2662290 | Human mRNA for cytochrome b5, partial cds. |
| 64 | hu00187718 | g2662290 | Human mRNA for cytochrome b5, partial cds. |
| 65 | hu00681549 | g2662290 | Human mRNA for cytochrome b5, partial cds. |
| 66 | hu00339586 | g2662290 | Human mRNA for cytochrome b5, partial cds. |
| 67 | hu01022298 | g3881842 | Similarity to E. coli 2-oxoglutarate dehydrogenase (SW:ODO1_ECOLI); cDNA EST |
| 68 | hu01122008 | g531404 | Human mRNA for glutaredoxin. |
| 69 | hu00228730 | g2351036 | Human mRNA for cytochrome b small subunit of complex II, complete cds. |
| 70 | hu01259066 | g3603309 | Human glutaredoxin mRNA, complete cds. |
| 71 | hu01271939 | g3603309 | Human glutaredoxin mRNA, complete cds. |
| 72 | hu01144669 | g2961254 | thioredoxin homolog |
| 73 | hu01312454 | g2114207 | glutaredoxin |
| 74 | hu00986823 | g5295994 | small subunit of cytochrome b of succinate dehydrogenase |
| 75 | hu01207133 | g5295994 | small subunit of cytochrome b of succinate dehydrogenase |
| 76 | hu01060525 | g5295994 | small subunit of cytochrome b of succinate dehydrogenase |
| 77 | hu01108730 | g5295994 | small subunit of cytochrome b of succinate dehydrogenase |
| 78 | hu01094006 | g5295994 | small subunit of cytochrome b of succinate dehydrogenase |
| 79 | hu01055702 | g5295994 | small subunit of cytochrome b of succinate dehydrogenase |
| 80 | hu01200549 | g5295994 | small subunit of cytochrome b of succinate dehydrogenase |
| 81 | hu01256253 | g5295994 | small subunit of cytochrome b of succinate dehydrogenase |
| 82 | hu01029382 | g5295994 | small subunit of cytochrome b of succinate dehydrogenase |
| 83 | hu01009078 | g5295994 | small subunit of cytochrome b of succinate dehydrogenase |
| 84 | hu01321595 | g5295994 | small subunit of cytochrome b of succinate dehydrogenase |
| 85 | hu01164609 | g5295994 | small subunit of cytochrome b of succinate dehydrogenase |
| 86 | hu01253674 | g5295994 | small subunit of cytochrome b of succinate dehydrogenase |
| 87 | hu01169353 | g5295994 | small subunit of cytochrome b of succinate dehydrogenase |
| 88 | hu01206885 | g5295994 | small subunit of cytochrome b of succinate dehydrogenase |
| 89 | hu01348582 | g5295994 | small subunit of cytochrome b of succinate dehydrogenase |
| 90 | hu01338934 | g5295994 | small subunit of cytochrome b of succinate dehydrogenase |
| 91 | hu01164479 | g5295994 | small subunit of cytochrome b of succinate dehydrogenase |
| 92 | hu01287961 | g5295994 | small subunit of cytochrome b of succinate dehydrogenase |
| 93 | hu01236008 | g1911548 | cytochrome c-like polypeptide [human, lung adenocarcinoma A549, Peptide, 190 aa] |
| 94 | rat00086191 | g2911066 | adrenodoxin-like protein |
| 95 | rat00096533 | g2911066 | adrenodoxin-like protein |
| 96 | rat00191537 | g2911066 | adrenodoxin-like protein |
| 97 | rat00075375 | g2911066 | adrenodoxin-like protein |
| 98 | rat00219672 | g2911066 | adrenodoxin-like protein |
| 99 | rat00223885 | g2911066 | adrenodoxin-like protein |
| 100 | rat00247348 | g2911066 | adrenodoxin-like protein |
| 101 | rat00069166 | g2911066 | adrenodoxin-like protein |
| 102 | rat00038669 | g2911066 | adrenodoxin-like protein |
| 103 | rat00205020 | g2911066 | adrenodoxin-like protein |
| 104 | rat00000073 | g2911066 | adrenodoxin-like protein |
| 105 | rat00221180 | g2911066 | adrenodoxin-like protein |
| 106 | rat00008019 | g2911066 | adrenodoxin-like protein |
| 107 | rat00033426 | g2911066 | adrenodoxin-like protein |
| 108 | rat00131752 | g2911066 | adrenodoxin-like protein |
| 109 | rat00230675 | g2911066 | adrenodoxin-like protein |
| 110 | rat00056283 | g2911066 | adrenodoxin-like protein |
| 111 | rat00144276 | g2911066 | adrenodoxin-like protein |
| 112 | hu01337947 | g1911548 | cytochrome c-like polypeptide [human, lung adenocarcinoma A549, Peptide, 190 aa] |
| 113 | hu01310032 | g1911548 | cytochrome c-like polypeptide [human, lung adenocarcinoma A549, Peptide, 190 aa] |
| 114 | hu00987948 | g1911547 | cytochrome c-like polypeptide [Human, lung adenocarcinoma A549, mRNA, 1041 nt]. |
| 115 | hu01216237 | g1911547 | cytochrome c-like polypeptide [Human, lung adenocarcinoma A549, mRNA, 1041 nt]. |
| 116 | hu01032720 | g1911547 | cytochrome c-like polypeptide [Human, lung adenocarcinoma A549, mRNA, 1041 nt]. |
| 117 | hu01119107 | g435963 | thioredoxin-like protein |
| 118 | hu01347050 | g435963 | thioredoxin-like protein |
| 119 | hu01343361 | g435963 | thioredoxin-like protein |
| 120 | hu01104646 | g435963 | thioredoxin-like protein |
| 121 | hu01179389 | g435963 | thioredoxin-like protein |
| 122 | hu00969772 | g435963 | thioredoxin-like protein |
| 123 | hu00994747 | g435963 | thioredoxin-like protein |
| 124 | hu01209376 | g435963 | thioredoxin-like protein |
| 125 | hu01330016 | g435963 | thioredoxin-like protein |
| 126 | hu01175881 | g435963 | thioredoxin-like protein |
| 127 | hu01264927 | g435963 | thioredoxin-like protein |
| 128 | hu01121470 | g435963 | thioredoxin-like protein |
| 129 | hu00996037 | g435963 | thioredoxin-like protein |
| 130 | hu01052325 | g435963 | thioredoxin-like protein |
| 131 | hu01137559 | g1911547 | cytochrome c-like polypeptide [Human, lung adenocarcinoma A549, mRNA, 1041 nt]. |
| 132 | hu01092065 | g1911547 | cytochrome c-like polypeptide [Human, lung adenocarcinoma A549, mRNA, 1041 nt]. |
| 133 | hu01323636 | g1911547 | cytochrome c-like polypeptide [Human, lung adenocarcinoma A549, mRNA, 1041 nt]. |
| 134 | hu01060728 | g1911547 | cytochrome c-like polypeptide [Human, lung adenocarcinoma A549, mRNA, 1041 nt]. |
| 135 | hu01268836 | g1911547 | cytochrome c-like polypeptide [Human, lung adenocarcinoma A549, mRNA, 1041 nt]. |
| 136 | hu01222473 | g3881842 | Similarity to E. coli 2-oxoglutarate dehydrogenase (SW:ODO1_ECOLI); cDNA EST |
| 137 | hu01236994 | g3881842 | Similarity to E. coli 2-oxoglutarate dehydrogenase (SW:ODO1_ECOLI); cDNA EST |
| 138 | hu00308470 | g531404 | Human mRNA for glutaredoxin. |
| 139 | hu00267375 | g531404 | Human mRNA for glutaredoxin. |
| 140 | hu00473030 | g531404 | Human mRNA for glutaredoxin. |
| 141 | hu00173475 | g3603309 | Human glutaredoxin mRNA, complete cds. |
| 142 | hu00849020 | g3603309 | Human glutaredoxin mRNA, complete cds. |
| 143 | hu00168009 | g3603309 | Human glutaredoxin mRNA, complete cds. |
| 144 | hu00580866 | g3603309 | Human glutaredoxin mRNA, complete cds. |
| 145 | hu00927748 | g2588778 | Human mRNA for cytochrome b large subunit of complex II, complete cds. |
| 146 | hu00403005 | g2588778 | Human mRNA for cytochrome b large subunit of complex II, complete cds. |
| 147 | hu00869778 | g2588778 | Human mRNA for cytochrome b large subunit of complex II, complete cds. |
| 148 | hu00063607 | g2588778 | Human mRNA for cytochrome b large subunit of complex II, complete cds. |
| 149 | hu00203909 | g2588778 | Human mRNA for cytochrome b large subunit of complex II, complete cds. |
| 150 | hu00452472 | g2588778 | Human mRNA for cytochrome b large subunit of complex II, complete cds. |
| 151 | hu00831183 | g2588778 | Human mRNA for cytochrome b large subunit of complex II, complete cds. |
| 152 | hu00372084 | g2588778 | Human mRNA for cytochrome b large subunit of complex II, complete cds. |
| 153 | hu00796320 | g2588778 | Human mRNA for cytochrome b large subunit of complex II, complete cds. |
| 154 | hu00613827 | g2588778 | Human mRNA for cytochrome b large subunit of complex II, complete cds. |
| 155 | hu00653184 | g2588778 | Human mRNA for cytochrome b large subunit of complex II, complete cds. |
| 156 | hu00533558 | g2588778 | Human mRNA for cytochrome b large subunit of complex II, complete cds. |
| 157 | hu00578046 | g2588778 | Human mRNA for cytochrome b large subunit of complex II, complete cds. |
| 158 | hu00482792 | g2588778 | Human mRNA for cytochrome b large subunit of complex II, complete cds. |
| 159 | hu00471785 | g2588778 | Human mRNA for cytochrome b large subunit of complex II, complete cds. |
| 160 | hu00335877 | g2588778 | Human mRNA for cytochrome b large subunit of complex II, complete cds. |
| 161 | hu00527595 | g2588778 | Human mRNA for cytochrome b large subunit of complex II, complete cds. |
| 162 | hu00875185 | g2588778 | Human mRNA for cytochrome b large subunit of complex II, complete cds. |
| 163 | hu00912110 | g2588778 | Human mRNA for cytochrome b large subunit of complex II, complete cds. |
| 164 | hu00026601 | g2588778 | Human mRNA for cytochrome b large subunit of complex II, complete cds. |
| 165 | hu00103248 | g2588778 | Human mRNA for cytochrome b large subunit of complex II, complete cds. |
| 166 | hu00631611 | g2588778 | Human mRNA for cytochrome b large subunit of complex II, complete cds. |
| 167 | hu00401675 | g2588778 | Human mRNA for cytochrome b large subunit of complex II, complete cds. |
| 168 | hu00676022 | g2588778 | Human mRNA for cytochrome b large subunit of complex II, complete cds. |
| 169 | hu00950091 | g2588778 | Human mRNA for cytochrome b large subunit of complex II, complete cds. |
| 170 | hu00780712 | g2588778 | Human mRNA for cytochrome b large subunit of complex II, complete cds. |
| 171 | hu00392614 | g2588778 | Human mRNA for cytochrome b large subunit of complex II, complete cds. |
| 172 | hu00407933 | g2588778 | Human mRNA for cytochrome b large subunit of complex II, complete cds. |
| 173 | hu00759320 | g2588778 | Human mRNA for cytochrome b large subunit of complex II, complete cds. |
| 174 | hu00709083 | g2588778 | Human mRNA for cytochrome b large subunit of complex II, complete cds. |
| 175 | hu00848224 | g2588778 | Human mRNA for cytochrome b large subunit of complex II, complete cds. |
| 176 | hu00703783 | g2588778 | Human mRNA for cytochrome b large subunit of complex II, complete cds. |
| 177 | hu00805276 | g2588778 | Human mRNA for cytochrome b large subunit of complex II, complete cds. |
| 178 | hu00921782 | g2588778 | Human mRNA for cytochrome b large subunit of complex II, complete cds. |
| 179 | hu00335487 | g2588778 | Human mRNA for cytochrome b large subunit of complex II, complete cds. |
| 180 | hu00549507 | g2588778 | Human mRNA for cytochrome b large subunit of complex II, complete cds. |
| 181 | hu00888223 | g2588778 | Human mRNA for cytochrome b large subunit of complex II, complete cds. |
| 182 | hu00224887 | g2588778 | Human mRNA for cytochrome b large subunit of complex II, complete cds. |
| 183 | hu00935744 | g2588778 | Human mRNA for cytochrome b large subunit of complex II, complete cds. |
| 184 | hu00861918 | g2588778 | Human mRNA for cytochrome b large subunit of complex II, complete cds. |
| 185 | hu00534975 | g2588778 | Human mRNA for cytochrome b large subunit of complex II, complete cds. |
| 186 | hu00031360 | g2588778 | Human mRNA for cytochrome b large subunit of complex II, complete cds. |
| 187 | hu00281501 | g2588778 | Human mRNA for cytochrome b large subunit of complex II, complete cds. |
| 188 | hu00128107 | g2588778 | Human mRNA for cytochrome b large subunit of complex II, complete cds. |
| 189 | hu00272072 | g2588778 | Human mRNA for cytochrome b large subunit of complex II, complete cds. |
| 190 | hu00651107 | g2588778 | Human mRNA for cytochrome b large subunit of complex II, complete cds. |
| 191 | hu00930901 | g2588778 | Human mRNA for cytochrome b large subunit of complex II, complete cds. |
| 192 | hu00271706 | g2588778 | Human mRNA for cytochrome b large subunit of complex II, complete cds. |
| 193 | hu00008916 | g2588778 | Human mRNA for cytochrome b large subunit of complex II, complete cds. |
| 194 | hu00405925 | g2588778 | Human mRNA for cytochrome b large subunit of complex II, complete cds. |
| 195 | hu00283922 | g2588778 | Human mRNA for cytochrome b large subunit of complex II, complete cds. |
| 196 | hu00394641 | g2588778 | Human mRNA for cytochrome b large subunit of complex II, complete cds. |
| 197 | hu00741298 | g2588778 | Human mRNA for cytochrome b large subunit of complex II, complete cds. |
| 198 | hu00509990 | g2588778 | Human mRNA for cytochrome b large subunit of complex II, complete cds. |
| 199 | hu00391975 | g2588778 | Human mRNA for cytochrome b large subunit of complex II, complete cds. |
| 200 | hu00681668 | g2588778 | Human mRNA for cytochrome b large subunit of complex II, complete cds. |
| 201 | hu00157699 | g2588778 | Human mRNA for cytochrome b large subunit of complex II, complete cds. |
| 202 | hu00240223 | g2588778 | Human mRNA for cytochrome b large subunit of complex II, complete cds. |
| 203 | hu00842100 | g2588778 | Human mRNA for cytochrome b large subunit of complex II, complete cds. |
| 204 | hu00533420 | g2588778 | Human mRNA for cytochrome b large subunit of complex II, complete cds. |
| 205 | hu00211556 | g2588778 | Human mRNA for cytochrome b large subunit of complex II, complete cds. |
| 206 | hu00391700 | g2588778 | Human mRNA for cytochrome b large subunit of complex II, complete cds. |
| 207 | hu00622361 | g2588778 | Human mRNA for cytochrome b large subunit of complex II, complete cds. |
| 208 | hu00629111 | g2588778 | Human mRNA for cytochrome b large subunit of complex II, complete cds. |
| 209 | hu00757004 | g2588778 | Human mRNA for cytochrome b large subunit of complex II, complete cds. |
| 210 | hu00382550 | g2588778 | Human mRNA for cytochrome b large subunit of complex II, complete cds. |
| 211 | hu00516056 | g2588778 | Human mRNA for cytochrome b large subunit of complex II, complete cds. |
| 212 | hu00268304 | g2588778 | Human mRNA for cytochrome b large subunit of complex II, complete cds. |
| 213 | hu00878200 | g2588778 | Human mRNA for cytochrome b large subunit of complex II, complete cds. |
| 214 | hu00235276 | g2588778 | Human mRNA for cytochrome b large subunit of complex II, complete cds. |
| 215 | hu00652761 | g2588778 | Human mRNA for cytochrome b large subunit of complex II, complete cds. |
| 216 | hu00008766 | g2588778 | Human mRNA for cytochrome b large subunit of complex II, complete cds. |
| 217 | hu00076745 | g2588778 | Human mRNA for cytochrome b large subunit of complex II, complete cds. |
| 218 | hu00177708 | g2588778 | Human mRNA for cytochrome b large subunit of complex II, complete cds. |
| 219 | hu00588913 | g2588778 | Human mRNA for cytochrome b large subunit of complex II, complete cds. |
| 220 | hu01070024 | g3646127 | Human mRNA for putative thioredoxin-like protein. |
| 221 | hu00173405 | g531404 | Human mRNA for glutaredoxin. |
| 222 | hu00039514 | g531404 | Human mRNA for glutaredoxin. |
| 223 | hu00026942 | g531404 | Human mRNA for glutaredoxin. |
| 224 | hu00187054 | g531404 | Human mRNA for glutaredoxin. |
| 225 | hu00854314 | g531404 | Human mRNA for glutaredoxin. |
| 226 | hu00885122 | g531404 | Human mRNA for glutaredoxin. |
| 227 | hu00217359 | g531404 | Human mRNA for glutaredoxin. |
| 228 | hu00384526 | g531404 | Human mRNA for glutaredoxin. |
| 229 | rat00071677 | g2443331 | Nfrl |
| 230 | rat00105537 | g2443331 | Nfrl |
| 231 | rat00177349 | g2443331 | Nfrl |
| 232 | rat00068188 | g2443331 | Nfrl |
| 233 | rat00145437 | g2443331 | Nfrl |
| 234 | hu01349846 | g3646127 | Human mRNA for putative thioredoxin-like protein. |
| 235 | hu01009468 | g3646127 | Human mRNA for putative thioredoxin-like protein. |
| 236 | hu01160420 | g3646127 | Human mRNA for putative thioredoxin-like protein. |
| 237 | hu01299909 | g3646127 | Human mRNA for putative thioredoxin-like protein. |
| 238 | hu01246478 | g3646127 | Human mRNA for putative thioredoxin-like protein. |
| 239 | hu01285278 | g3646127 | Human mRNA for putative thioredoxin-like protein. |
| 240 | hu01264445 | g3646127 | Human mRNA for putative thioredoxin-like protein. |
| 241 | hu00198187 | g181260 | Human somatic cytochrome c (HC5) processed pseudogene, complete cds. |
| 242 | hu00350676 | g181228 | Human cytochrome b5 mRNA, 3′ end. |
| 243 | hu00837746 | g2702453 | contains similarity to thioredoxin domains |
| 244 | hu00818732 | g2702453 | contains similarity to thioredoxin domains |
| 245 | hu00171330 | g2702453 | contains similarity to thioredoxin domains |
| 246 | hu00471838 | g2702453 | contains similarity to thioredoxin domains |
| 247 | hu00496995 | g2702453 | contains similarity to thioredoxin domains |
| 248 | hu00645541 | g5295993 | Human SDHD gene for small subunit of cytochrome b of succinate dehydrogenase, complete cds. |
| 249 | hu00696057 | g5295993 | Human SDHD gene for small subunit of cytochrome b of succinate dehydrogenase, complete cds. |
| 250 | hu00792990 | g2702453 | contains similarity to thioredoxin domains |
| 251 | hu00549929 | g2702453 | contains similarity to thioredoxin domains |
| 252 | hu00897882 | g2702453 | contains similarity to thioredoxin domains |
| 253 | hu00016152 | g2702453 | contains similarity to thioredoxin domains |
| 254 | hu00103257 | g2702453 | contains similarity to thioredoxin domains |
| 255 | hu00123714 | g2702453 | contains similarity to thioredoxin domains |
| 256 | hu00552910 | g2702453 | contains similarity to thioredoxin domains |
| 257 | hu00855123 | g2702453 | contains similarity to thioredoxin domains |
| 258 | hu00854305 | g2702453 | contains similarity to thioredoxin domains |
| 259 | hu00397024 | g2702453 | contains similarity to thioredoxin domains |
| 260 | hu00143648 | g2702453 | contains similarity to thioredoxin domains |
| 261 | hu00705662 | g2702453 | contains similarity to thioredoxin domains |
| 262 | hu00593090 | g2702453 | contains similarity to thioredoxin domains |
| 263 | hu00459622 | g2702453 | contains similarity to thioredoxin domains |
| 264 | hu00417548 | g2702453 | contains similarity to thioredoxin domains |
| 265 | hu00076844 | g2702453 | contains similarity to thioredoxin domains |
| 266 | hu00758235 | g2702453 | contains similarity to thioredoxin domains |
| 267 | hu00339267 | g2702453 | contains similarity to thioredoxin domains |
| 268 | hu00166935 | g2702453 | contains similarity to thioredoxin domains |
| 269 | hu00942197 | g2702453 | contains similarity to thioredoxin domains |
| 270 | hu00525849 | g2702453 | contains similarity to thioredoxin domains |
| 271 | hu00668292 | g2702453 | contains similarity to thioredoxin domains |
| 272 | hu00096927 | g2702453 | contains similarity to thioredoxin domains |
| 273 | hu00868174 | g2702453 | contains similarity to thioredoxin domains |
| 274 | hu00001500 | g2351036 | Human mRNA for cytochrome b small subunit of complex II, complete cds. |
| 275 | hu00598479 | g2351036 | Human mRNA for cytochrome b small subunit of complex II, complete cds. |
| 276 | hu00576699 | g2351036 | Human mRNA for cytochrome b small subunit of complex II, complete cds. |
| 277 | hu00115451 | g2351036 | Human mRNA for cytochrome b small subunit of complex II, complete cds. |
| 278 | hu00063441 | g2351036 | Human mRNA for cytochrome b small subunit of complex II, complete cds. |
| 279 | hu00117073 | g2351036 | Human mRNA for cytochrome b small subunit of complex II, complete cds. |
| 280 | hu00034373 | g2351036 | Human mRNA for cytochrome b small subunit of complex II, complete cds. |
| 281 | hu00321357 | g2351036 | Human mRNA for cytochrome b small subunit of complex II, complete cds. |
| 282 | hu00606532 | g2351036 | Human mRNA for cytochrome b small subunit of complex II, complete cds. |
| 283 | hu00331079 | g2351036 | Human mRNA for cytochrome b small subunit of complex II, complete cds. |
| 284 | hu00091149 | g2351036 | Human mRNA for cytochrome b small subunit of complex II, complete cds. |
| 285 | hu00390348 | g2351036 | Human mRNA for cytochrome b small subunit of complex II, complete cds. |
| 286 | hu00743094 | g2351036 | Human mRNA for cytochrome b small subunit of complex II, complete cds. |
| 287 | hu00652935 | g2351036 | Human mRNA for cytochrome b small subunit of complex II, complete cds. |
| 288 | hu00639806 | g2351036 | Human mRNA for cytochrome b small subunit of complex II, complete cds. |
| 289 | hu00661686 | g2351036 | Human mRNA for cytochrome b small subunit of complex II, complete cds. |
| 290 | hu00698038 | g2351036 | Human mRNA for cytochrome b small subunit of complex II, complete cds. |
| 291 | hu00942589 | g2351036 | Human mRNA for cytochrome b small subunit of complex II, complete cds. |
| 292 | hu00689928 | g2351036 | Human mRNA for cytochrome b small subunit of complex II, complete cds. |
| 293 | hu00201651 | g2351036 | Human mRNA for cytochrome b small subunit of complex II, complete cds. |
| 294 | hu00616103 | g2351036 | Human mRNA for cytochrome b small subunit of complex II, complete cds. |
| 295 | hu00300158 | g2351036 | Human mRNA for cytochrome b small subunit of complex II, complete cds. |
| 296 | hu00534530 | g2351036 | Human mRNA for cytochrome b small subunit of complex II, complete cds. |
| 297 | hu00924051 | g2351036 | Human mRNA for cytochrome b small subunit of complex II, complete cds. |
| 298 | hu00256307 | g2351036 | Human mRNA for cytochrome b small subunit of complex II, complete cds. |
| 299 | hu00114972 | g2351036 | Human mRNA for cytochrome b small subunit of complex II, complete cds. |
| 300 | hu00330198 | g2351036 | Human mRNA for cytochrome b small subunit of complex II, complete cds. |
| 301 | hu00559222 | g2351036 | Human mRNA for cytochrome b small subunit of complex II, complete cds. |
| 302 | hu00257435 | g2351036 | Human mRNA for cytochrome b small subunit of complex II, complete cds. |
| 303 | hu00641771 | g2351036 | Human mRNA for cytochrome b small subunit of complex II, complete cds. |
| 304 | hu00157481 | g2351036 | Human mRNA for cytochrome b small subunit of complex II, complete cds. |
| 305 | hu00649531 | g2351036 | Human mRNA for cytochrome b small subunit of complex II, complete cds. |
| 306 | hu00256466 | g2351036 | Human mRNA for cytochrome b small subunit of complex II, complete cds. |
| 307 | hu00637950 | g2351036 | Human mRNA for cytochrome b small subunit of complex II, complete cds. |
| 308 | hu00891318 | g2351036 | Human mRNA for cytochrome b small subunit of complex II, complete cds. |
| 309 | hu00267556 | g2351036 | Human mRNA for cytochrome b small subunit of complex II, complete cds. |
| 310 | hu00189229 | g2351036 | Human mRNA for cytochrome b small subunit of complex II, complete cds. |
| 311 | hu00803857 | g2351036 | Human mRNA for cytochrome b small subunit of complex II, complete cds. |
| 312 | hu00428667 | g2351036 | Human mRNA for cytochrome b small subunit of complex II, complete cds. |
| 313 | hu00593649 | g2351036 | Human mRNA for cytochrome b small subunit of complex II, complete cds. |
| 314 | hu00966556 | g2351036 | Human mRNA for cytochrome b small subunit of complex II, complete cds. |
| 315 | hu00908022 | g2351036 | Human mRNA for cytochrome b small subunit of complex II, complete cds. |
| 316 | hu00247720 | g2351036 | Human mRNA for cytochrome b small subunit of complex II, complete cds. |
| 317 | hu00093263 | g2351036 | Human mRNA for cytochrome b small subunit of complex II, complete cds. |
| 318 | hu00384521 | g2351036 | Human mRNA for cytochrome b small subunit of complex II, complete cds. |
| 319 | hu00322584 | g2351036 | Human mRNA for cytochrome b small subunit of complex II, complete cds. |
| 320 | hu00670453 | g2351036 | Human mRNA for cytochrome b small subunit of complex II, complete cds. |
| 321 | hu00056338 | g2351036 | Human mRNA for cytochrome b small subunit of complex II, complete cds. |
| 322 | hu00377154 | g2351036 | Human mRNA for cytochrome b small subunit of complex II, complete cds. |
| 323 | hu00289424 | g2351036 | Human mRNA for cytochrome b small subunit of complex II, complete cds. |
| 324 | hu00138864 | g2351036 | Human mRNA for cytochrome b small subunit of complex II, complete cds. |
| 325 | hu00178785 | g2351036 | Human mRNA for cytochrome b small subunit of complex II, complete cds. |
| 326 | hu00886298 | g2351036 | Human mRNA for cytochrome b small subunit of complex II, complete cds. |
| 327 | hu00527500 | g2351036 | Human mRNA for cytochrome b small subunit of complex II, complete cds. |
| 328 | hu00642045 | g2351036 | Human mRNA for cytochrome b small subunit of complex II, complete cds. |
| 329 | hu00093117 | g2351036 | Human mRNA for cytochrome b small subunit of complex II, complete cds. |
| 330 | hu00614740 | g2351036 | Human mRNA for cytochrome b small subunit of complex II, complete cds. |
| 331 | hu00265154 | g2351036 | Human mRNA for cytochrome b small subunit of complex II, complete cds. |
| 332 | hu00105683 | g2351036 | Human mRNA for cytochrome b small subunit of complex II, complete cds. |
| 333 | hu00117267 | g2351036 | Human mRNA for cytochrome b small subunit of complex II, complete cds. |
| 334 | hu00552139 | g2351036 | Human mRNA for cytochrome b small subunit of complex II, complete cds. |
| 335 | hu00084843 | g2351036 | Human mRNA for cytochrome b small subunit of complex II, complete cds. |
| 336 | hu00633854 | g2351036 | Human mRNA for cytochrome b small subunit of complex II, complete cds. |
| 337 | hu00617818 | g2351036 | Human mRNA for cytochrome b small subunit of complex II, complete cds. |
| 338 | hu00168917 | g2351036 | Human mRNA for cytochrome b small subunit of complex II, complete cds. |
| 339 | hu00897711 | g2351036 | Human mRNA for cytochrome b small subunit of complex II, complete cds. |
| 340 | hu00850688 | g2351036 | Human mRNA for cytochrome b small subunit of complex II, complete cds. |
| 341 | hu00724100 | g2351036 | Human mRNA for cytochrome b small subunit of complex II, complete cds. |
| 342 | hu00017402 | g2351036 | Human mRNA for cytochrome b small subunit of complex II, complete cds. |
| 343 | hu00602053 | g2351036 | Human mRNA for cytochrome b small subunit of complex II, complete cds. |
| 344 | hu00521216 | g2351036 | Human mRNA for cytochrome b small subunit of complex II, complete cds. |
| 345 | hu00403523 | g2351036 | Human mRNA for cytochrome b small subunit of complex II, complete cds. |
| 346 | hu00075141 | g2351036 | Human mRNA for cytochrome b small subunit of complex II, complete cds. |
| 347 | hu00022437 | g2351036 | Human mRNA for cytochrome b small subunit of complex II, complete cds. |
| 348 | hu00201683 | g2351036 | Human mRNA for cytochrome b small subunit of complex II, complete cds. |
| 349 | hu00860016 | g2351036 | Human mRNA for cytochrome b small subunit of complex II, complete cds. |
| 350 | hu00642216 | g2351036 | Human mRNA for cytochrome b small subunit of complex II, complete cds. |
| 351 | hu00017647 | g2351036 | Human mRNA for cytochrome b small subunit of complex II, complete cds. |
| 352 | hu00336922 | g2351036 | Human mRNA for cytochrome b small subunit of complex II, complete cds. |
| 353 | hu00155892 | g2351036 | Human mRNA for cytochrome b small subunit of complex II, complete cds. |
| 354 | hu00827284 | g2351036 | Human mRNA for cytochrome b small subunit of complex II, complete cds. |
| 355 | hu00895708 | g2351036 | Human mRNA for cytochrome b small subunit of complex II, complete cds. |
| 356 | hu00219012 | g2351036 | Human mRNA for cytochrome b small subunit of complex II, complete cds. |
| 357 | hu00033057 | g2351036 | Human mRNA for cytochrome b small subunit of complex II, complete cds. |
| 358 | hu00729121 | g2351036 | Human mRNA for cytochrome b small subunit of complex II, complete cds. |
| 359 | hu00935501 | g2351036 | Human mRNA for cytochrome b small subunit of complex II, complete cds. |
| 360 | hu00644405 | g2351036 | Human mRNA for cytochrome b small subunit of complex II, complete cds. |
| 361 | hu00672261 | g2351036 | Human mRNA for cytochrome b small subunit of complex II, complete cds. |
| 362 | hu00681415 | g2351036 | Human mRNA for cytochrome b small subunit of complex II, complete cds. |
| 363 | hu01353645 | g2351036 | Human mRNA for cytochrome b small subunit of complex II, complete cds. |
| 364 | hu00155187 | g2351036 | Human mRNA for cytochrome b small subunit of complex II, complete cds. |
| 365 | hu00671536 | g2351036 | Human mRNA for cytochrome b small subunit of complex II, complete cds. |
| 366 | hu00664723 | g2351036 | Human mRNA for cytochrome b small subunit of complex II, complete cds. |
| 367 | hu00874109 | g2351036 | Human mRNA for cytochrome b small subunit of complex II, complete cds. |
| 368 | hu00515388 | g2351036 | Human mRNA for cytochrome b small subunit of complex II, complete cds. |
| 369 | hu00081957 | g2351036 | Human mRNA for cytochrome b small subunit of complex II, complete cds. |
| 370 | hu00688447 | g2351036 | Human mRNA for cytochrome b small subunit of complex II, complete cds. |
| 371 | hu00120308 | g2351036 | Human mRNA for cytochrome b small subunit of complex II, complete cds. |
| 372 | hu00700244 | g2351036 | Human mRNA for cytochrome b small subunit of complex II, complete cds. |
| 373 | hu00641024 | g2351036 | Human mRNA for cytochrome b small subunit of complex II, complete cds. |
| 374 | hu00445320 | g2351036 | Human mRNA for cytochrome b small subunit of complex II, complete cds. |
| 375 | hu00850230 | g2351036 | Human mRNA for cytochrome b small subunit of complex II, complete cds. |
| 376 | hu00381730 | g2351036 | Human mRNA for cytochrome b small subunit of complex II, complete cds. |
| 377 | hu00229540 | g2351036 | Human mRNA for cytochrome b small subunit of complex II, complete cds. |
| 378 | hu00439241 | g2351036 | Human mRNA for cytochrome b small subunit of complex II, complete cds. |
| 379 | hu00944105 | g2351036 | Human mRNA for cytochrome b small subunit of complex II, complete cds. |
| 380 | hu00107493 | g2351036 | Human mRNA for cytochrome b small subunit of complex II, complete cds. |
| 381 | hu00580182 | g2351036 | Human mRNA for cytochrome b small subunit of complex II, complete cds. |
| 382 | hu00682152 | g2351036 | Human mRNA for cytochrome b small subunit of complex II, complete cds. |
| 383 | hu00760642 | g2351036 | Human mRNA for cytochrome b small subunit of complex II, complete cds. |
| 384 | hu00742742 | g2351036 | Human mRNA for cytochrome b small subunit of complex II, complete cds. |
| 385 | hu00082296 | g2351036 | Human mRNA for cytochrome b small subunit of complex II, complete cds. |
| 386 | hu00647803 | g2351036 | Human mRNA for cytochrome b small subunit of complex II, complete cds. |
| 387 | hu00098155 | g2351036 | Human mRNA for cytochrome b small subunit of complex II, complete cds. |
| 388 | hu00540983 | g2351036 | Human mRNA for cytochrome b small subunit of complex II, complete cds. |
| 389 | hu00823826 | g2351036 | Human mRNA for cytochrome b small subunit of complex II, complete cds. |
| 390 | hu00316469 | g2351036 | Human mRNA for cytochrome b small subunit of complex II, complete cds. |
| 391 | hu00186680 | g2351036 | Human mRNA for cytochrome b small subunit of complex II, complete cds. |
| 392 | hu00192684 | g2351036 | Human mRNA for cytochrome b small subunit of complex II, complete cds. |
| 393 | hu00509719 | g2351036 | Human mRNA for cytochrome b small subunit of complex II, complete cds. |
| 394 | hu00690657 | g2351036 | Human mRNA for cytochrome b small subunit of complex II, complete cds. |
| 395 | hu00319777 | g2351036 | Human mRNA for cytochrome b small subunit of complex II, complete cds. |
| 396 | hu00259981 | g2351036 | Human mRNA for cytochrome b small subunit of complex II, complete cds. |
| 397 | hu00555399 | g2351036 | Human mRNA for cytochrome b small subunit of complex II, complete cds. |
| 398 | hu00312115 | g2351036 | Human mRNA for cytochrome b small subunit of complex II, complete cds. |
| 399 | hu00663211 | g2351036 | Human mRNA for cytochrome b small subunit of complex II, complete cds. |
| 400 | hu00322633 | g2351036 | Human mRNA for cytochrome b small subunit of complex II, complete cds. |
| 401 | hu00262441 | g2351036 | Human mRNA for cytochrome b small subunit of complex II, complete cds. |
| 402 | hu00441056 | g2351036 | Human mRNA for cytochrome b small subunit of complex II, complete cds. |
| 403 | hu00284223 | g2351036 | Human mRNA for cytochrome b small subunit of complex II, complete cds. |
| 404 | hu00103469 | g2351036 | Human mRNA for cytochrome b small subunit of complex II, complete cds. |
| 405 | hu00133746 | g2351036 | Human mRNA for cytochrome b small subunit of complex II, complete cds. |
| 406 | hu00224944 | g2351036 | Human mRNA for cytochrome b small subunit of complex II, complete cds. |
| 407 | hu00785500 | g2351036 | Human mRNA for cytochrome b small subunit of complex II, complete cds. |
| 408 | hu00252881 | g2351036 | Human mRNA for cytochrome b small subunit of complex II, complete cds. |
| 409 | hu00458208 | g2351036 | Human mRNA for cytochrome b small subunit of complex II, complete cds. |
| 410 | hu00298622 | g2351036 | Human mRNA for cytochrome b small subunit of complex II, complete cds. |
| 411 | hu00484049 | g2351036 | Human mRNA for cytochrome b small subunit of complex II, complete cds. |
| 412 | hu00911883 | g2351036 | Human mRNA for cytochrome b small subunit of complex II, complete cds. |
| 413 | hu00591366 | g2351036 | Human mRNA for cytochrome b small subunit of complex II, complete cds. |
| 414 | hu00189607 | g2351036 | Human mRNA for cytochrome b small subunit of complex II, complete cds. |
| 415 | hu00901849 | g2351036 | Human mRNA for cytochrome b small subunit of complex II, complete cds. |
| 416 | hu00576852 | g2351036 | Human mRNA for cytochrome b small subunit of complex II, complete cds. |
| 417 | hu00262689 | g2351036 | Human mRNA for cytochrome b small subunit of complex II, complete cds. |
| 418 | hu00194559 | g2351036 | Human mRNA for cytochrome b small subunit of complex II, complete cds. |
| 419 | hu00095974 | g2351036 | Human mRNA for cytochrome b small subunit of complex II, complete cds. |
| 420 | hu00399290 | g1911547 | cytochrome c-like polypeptide [Human, lung adenocarcinoma A549, mRNA, 1041 nt]. |
| 421 | hu01329355 | g453964 | ATL-derived factor/thioredoxin |
| 422 | hu00529652 | g3881842 | Similarity to E. coli 2-oxoglutarate dehydrogenase (SW:ODO1_ECOLI); cDNA EST |
| 423 | hu01054127 | g181232 | Human p22-phox (CYBA) gene, exon 2. |
| 424 | hu00367261 | g1667354 | electron-transfer flavoprotein alpha-subunit |
| 425 | hu01216944 | g2911066 | adrenodoxin-like protein |
| 426 | hu01169541 | g2911066 | adrenodoxin-like protein |
| 427 | hu00968022 | g2911066 | adrenodoxin-like protein |
| 428 | hu01042903 | g2911066 | adrenodoxin-like protein |
| 429 | hu01273836 | g2911066 | adrenodoxin-like protein |
| 430 | hu01270499 | g2911066 | adrenodoxin-like protein |
| 431 | hu01033738 | g2911066 | adrenodoxin-like protein |
| 432 | hu01201032 | g2911066 | adrenodoxin-like protein |
| 433 | hu00524021 | g703080 | Human cytochrome b5 (CYB5) gene, exon 5. |
| 434 | hu00364988 | g703080 | Human cytochrome b5 (CYB5) gene, exon 5. |
| 435 | hu00932363 | g703080 | Human cytochrome b5 (CYB5) gene, exon 5. |
| 436 | hu01103446 | g2443331 | Nfrl |
| 437 | hu01203049 | g2443331 | Nfrl |
| 438 | hu01068510 | g2443331 | Nfrl |
| 439 | hu01213943 | g2443331 | Nfrl |
| 440 | hu01044863 | g2443331 | Nfrl |
| 441 | hu01200968 | g2443331 | Nfrl |
| 442 | hu01018101 | g2443331 | Nfrl |
| 443 | hu01329733 | g2443331 | Nfrl |
| 444 | hu01337095 | g2443331 | Nfrl |
| 445 | hu01282087 | g2443331 | Nfrl |
| 446 | hu01090767 | g2443331 | Nfrl |
| 447 | hu00979175 | g5295993 | Human SDHD gene for small subunit of cytochrome b of succinate dehydrogenase, complete cds. |
| 448 | hu01182918 | g5295993 | Human SDHD gene for small subunit of cytochrome b of succinate dehydrogenase, complete cds. |
| 449 | hu01343318 | g5295993 | Human SDHD gene for small subunit of cytochrome b of succinate dehydrogenase, complete cds. |
| 450 | hu01147865 | g5295993 | Human SDHD gene for small subunit of cytochrome b of succinate dehydrogenase, complete cds. |
| 451 | hu01074751 | g5295993 | Human SDHD gene for small subunit of cytochrome b of succinate dehydrogenase, complete cds. |
| 452 | hu01296205 | g5295993 | Human SDHD gene for small subunit of cytochrome b of succinate dehydrogenase, complete cds. |
| 453 | hu00970451 | g5295993 | Human SDHD gene for small subunit of cytochrome b of succinate dehydrogenase, complete cds. |
| 454 | hu01325064 | g2443331 | Nfrl |
| 455 | hu01065608 | g2443331 | Nfrl |
| 456 | hu01046405 | g5295993 | Human SDHD gene for small subunit of cytochrome b of succinate dehydrogenase, complete cds. |
| 457 | hu01285201 | g5295993 | Human SDHD gene for small subunit of cytochrome b of succinate dehydrogenase, complete cds. |
| 458 | hu01013366 | g5295993 | Human SDHD gene for small subunit of cytochrome b of succinate dehydrogenase, complete cds. |
| 459 | hu01326433 | g5295993 | Human SDHD gene for small subunit of cytochrome b of succinate dehydrogenase, complete cds. |
| 460 | hu01147225 | g5295993 | Human SDHD gene for small subunit of cytochrome b of succinate dehydrogenase, complete cds. |
| 461 | hu01344254 | g5295993 | Human SDHD gene for small subunit of cytochrome b of succinate dehydrogenase, complete cds. |
| 462 | hu01128192 | g5295993 | Human SDHD gene for small subunit of cytochrome b of succinate dehydrogenase, complete cds. |
| 463 | hu01067191 | g5295993 | Human SDHD gene for small subunit of cytochrome b of succinate dehydrogenase, complete cds. |
| 464 | hu01070449 | g5295993 | Human SDHD gene for small subunit of cytochrome b of succinate dehydrogenase, complete cds. |
| 465 | hu01324078 | g5295993 | Human SDHD gene for small subunit of cytochrome b of succinate dehydrogenase, complete cds. |
| 466 | hu01051012 | g5295993 | Human SDHD gene for small subunit of cytochrome b of succinate dehydrogenase, complete cds. |
| 467 | hu01324557 | g5295993 | Human SDHD gene for small subunit of cytochrome b of succinate dehydrogenase, complete cds. |
| 468 | hu01070929 | g5295993 | Human SDHD gene for small subunit of cytochrome b of succinate dehydrogenase, complete cds. |
| 469 | hu00983044 | g5295993 | Human SDHD gene for small subunit of cytochrome b of succinate dehydrogenase, complete cds. |
| 470 | hu01170686 | g5295993 | Human SDHD gene for small subunit of cytochrome b of succinate dehydrogenase, complete cds. |
| 471 | hu00968530 | g5295993 | Human SDHD gene for small subunit of cytochrome b of succinate dehydrogenase, complete cds. |
| 472 | hu00010006 | g2662290 | Human mRNA for cytochrome b5, partial cds. |
| 473 | hu00957158 | g790540 | cytochrome B561 |
| 474 | hu01264231 | g2443331 | Nfrl |
| 475 | hu00305008 | g2662290 | Human mRNA for cytochrome b5, partial cds. |
| 476 | hu00631844 | g2662290 | Human mRNA for cytochrome b5, partial cds. |
| 477 | hu00667379 | g2662290 | Human mRNA for cytochrome b5, partial cds. |
| 478 | hu00200113 | g2662290 | Human mRNA for cytochrome b5, partial cds. |
| 479 | hu00657975 | g2662290 | Human mRNA for cytochrome b5, partial cds. |
| 480 | hu00090130 | g2662290 | Human mRNA for cytochrome b5, partial cds. |
| 481 | hu00731030 | g2662290 | Human mRNA for cytochrome b5, partial cds. |
| 482 | hu00112740 | g2662290 | Human mRNA for cytochrome b5, partial cds. |
| 483 | hu00405180 | g2662290 | Human mRNA for cytochrome b5, partial cds. |
| 484 | hu00801145 | g2662290 | Human mRNA for cytochrome b5, partial cds. |
| 485 | hu00710542 | g2662290 | Human mRNA for cytochrome b5, partial cds. |
| 486 | hu00141175 | g2662290 | Human mRNA for cytochrome b5, partial cds. |
| 487 | hu00414290 | g2662290 | Human mRNA for cytochrome b5, partial cds. |
| 488 | hu00264315 | g2662290 | Human mRNA for cytochrome b5, partial cds. |
| 489 | hu00049062 | g2662290 | Human mRNA for cytochrome b5, partial cds. |
| 490 | hu00059621 | g2662290 | Human mRNA for cytochrome b5, partial cds. |
| 491 | hu00166903 | g2662290 | Human mRNA for cytochrome b5, partial cds. |
| 492 | hu00503917 | g2662290 | Human mRNA for cytochrome b5, partial cds. |
| 493 | hu00134926 | g2662290 | Human mRNA for cytochrome b5, partial cds. |
| 494 | hu00445742 | g2662290 | Human mRNA for cytochrome b5, partial cds. |
| 495 | hu00336087 | g2662290 | Human mRNA for cytochrome b5, partial cds. |
| 496 | hu00929610 | g2662290 | Human mRNA for cytochrome b5, partial cds. |
| 497 | hu00079146 | g2662290 | Human mRNA for cytochrome b5, partial cds. |
| 498 | hu00273268 | g2662290 | Human mRNA for cytochrome b5, partial cds. |
| 499 | hu00628912 | g2662290 | Human mRNA for cytochrome b5, partial cds. |
| 500 | hu00086716 | g2662290 | Human mRNA for cytochrome b5, partial cds. |
| 501 | hu00614476 | g2662290 | Human mRNA for cytochrome b5, partial cds. |
| 502 | hu00148645 | g2662290 | Human mRNA for cytochrome b5, partial cds. |
| 503 | hu00833681 | g2662290 | Human mRNA for cytochrome b5, partial cds. |
| 504 | hu00532388 | g2662290 | Human mRNA for cytochrome b5, partial cds. |
| 505 | hu00436439 | g2662290 | Human mRNA for cytochrome b5, partial cds. |
| 506 | hu00088932 | g2662290 | Human mRNA for cytochrome b5, partial cds. |
| 507 | hu00149178 | g2662290 | Human mRNA for cytochrome b5, partial cds. |
| 508 | hu00426594 | g2662290 | Human mRNA for cytochrome b5, partial cds. |
| 509 | hu00065947 | g2662290 | Human mRNA for cytochrome b5, partial cds. |
| 510 | hu00915854 | g2662290 | Human mRNA for cytochrome b5, partial cds. |
| 511 | hu00944169 | g2662290 | Human mRNA for cytochrome b5, partial cds. |
| 512 | hu00775518 | g2662290 | Human mRNA for cytochrome b5, partial cds. |
| 513 | hu00369357 | g2662290 | Human mRNA for cytochrome b5, partial cds. |
| 514 | hu00668950 | g2662290 | Human mRNA for cytochrome b5, partial cds. |
| 515 | hu00891829 | g2662290 | Human mRNA for cytochrome b5, partial cds. |
| 516 | hu00036080 | g2662290 | Human mRNA for cytochrome b5, partial cds. |
| 517 | hu00904410 | g2662290 | Human mRNA for cytochrome b5, partial cds. |
| 518 | hu00505381 | g2662290 | Human mRNA for cytochrome b5, partial cds. |
| 519 | hu00431437 | g2662290 | Human mRNA for cytochrome b5, partial cds. |
| 520 | hu00851911 | g2662290 | Human mRNA for cytochrome b5, partial cds. |
| 521 | hu00044828 | g2662290 | Human mRNA for cytochrome b5, partial cds. |
| 522 | hu00562591 | g2662290 | Human mRNA for cytochrome b5, partial cds. |
| 523 | hu00682264 | g2662290 | Human mRNA for cytochrome b5, partial cds. |
| 524 | hu00505828 | g2662290 | Human mRNA for cytochrome b5, partial cds. |
| 525 | hu00881192 | g2662290 | Human mRNA for cytochrome b5, partial cds. |
| 526 | hu00486165 | g2662290 | Human mRNA for cytochrome b5, partial cds. |
| 527 | hu00741963 | g2662290 | Human mRNA for cytochrome b5, partial cds. |
| 528 | hu00424866 | g2662290 | Human mRNA for cytochrome b5, partial cds. |
| 529 | hu00575940 | g2662290 | Human mRNA for cytochrome b5, partial cds. |
| 530 | hu00952636 | g2662290 | Human mRNA for cytochrome b5, partial cds. |
| 531 | hu00777181 | g2662290 | Human mRNA for cytochrome b5, partial cds. |
| 532 | hu00820669 | g2662290 | Human mRNA for cytochrome b5, partial cds. |
| 533 | hu00950657 | g2662290 | Human mRNA for cytochrome b5, partial cds. |
| 534 | hu00492926 | g2662290 | Human mRNA for cytochrome b5, partial cds. |
| 535 | hu00386691 | g2662290 | Human mRNA for cytochrome b5, partial cds. |
| 536 | hu00691935 | g2662290 | Human mRNA for cytochrome b5, partial cds. |
| 537 | hu00672864 | g2662290 | Human mRNA for cytochrome b5, partial cds. |
| 538 | hu00824866 | g2662290 | Human mRNA for cytochrome b5, partial cds. |
| 539 | hu00849554 | g2662290 | Human mRNA for cytochrome b5, partial cds. |
| 540 | hu00694880 | g2662290 | Human mRNA for cytochrome b5, partial cds. |
| 541 | hu00488548 | g2662290 | Human mRNA for cytochrome b5, partial cds. |
| 542 | hu00861598 | g2662290 | Human mRNA for cytochrome b5, partial cds. |
| 543 | hu00748875 | g2662290 | Human mRNA for cytochrome b5, partial cds. |
| 544 | hu00430192 | g2662290 | Human mRNA for cytochrome b5, partial cds. |
| 545 | hu00001649 | g2662290 | Human mRNA for cytochrome b5, partial cds. |
| 546 | hu00182128 | g2662290 | Human mRNA for cytochrome b5, partial cds. |
| 547 | hu00550066 | g2662290 | Human mRNA for cytochrome b5, partial cds. |
| 548 | hu00496130 | g2662290 | Human mRNA for cytochrome b5, partial cds. |
| 549 | hu00515714 | g2662290 | Human mRNA for cytochrome b5, partial cds. |
| 550 | hu00723751 | g2662290 | Human mRNA for cytochrome b5, partial cds. |
| 551 | hu00150946 | g2662290 | Human mRNA for cytochrome b5, partial cds. |
| 552 | hu00111878 | g2662290 | Human mRNA for cytochrome b5, partial cds. |
| 553 | hu00128465 | g2662290 | Human mRNA for cytochrome b5, partial cds. |
| 554 | hu00561474 | g2662290 | Human mRNA for cytochrome b5, partial cds. |
| 555 | hu00279622 | g2662290 | Human mRNA for cytochrome b5, partial cds. |
| 556 | hu00895600 | g2662290 | Human mRNA for cytochrome b5, partial cds. |
| 557 | hu00139834 | g2662290 | Human mRNA for cytochrome b5, partial cds. |
| 558 | hu00872570 | g2662290 | Human mRNA for cytochrome b5, partial cds. |
| 559 | hu00624854 | g2662290 | Human mRNA for cytochrome b5, partial cds. |
| 560 | hu00571395 | g2662290 | Human mRNA for cytochrome b5, partial cds. |
| 561 | hu00872139 | g2662290 | Human mRNA for cytochrome b5, partial cds. |
| 562 | hu00498235 | g2662290 | Human mRNA for cytochrome b5, partial cds. |
| 563 | hu00736942 | g2662290 | Human mRNA for cytochrome b5, partial cds. |
| 564 | hu00437945 | g2662290 | Human mRNA for cytochrome b5, partial cds. |
| 565 | hu00677247 | g2662290 | Human mRNA for cytochrome b5, partial cds. |
| 566 | hu00287089 | g2662290 | Human mRNA for cytochrome b5, partial cds. |
| 567 | hu00893466 | g2662290 | Human mRNA for cytochrome b5, partial cds. |
| 568 | hu00539398 | g2662290 | Human mRNA for cytochrome b5, partial cds. |
| 569 | hu00856774 | g2662290 | Human mRNA for cytochrome b5, partial cds. |
| 570 | hu00692996 | g2662290 | Human mRNA for cytochrome b5, partial cds. |
| 571 | hu00966768 | g2662290 | Human mRNA for cytochrome b5, partial cds. |
| 572 | hu00030404 | g2662290 | Human mRNA for cytochrome b5, partial cds. |
| 573 | hu00332510 | g2662290 | Human mRNA for cytochrome b5, partial cds. |
| 574 | hu00650103 | g2662290 | Human mRNA for cytochrome b5, partial cds. |
| 575 | hu00003951 | g2662290 | Human mRNA for cytochrome b5, partial cds. |
| 576 | hu00560152 | g2662290 | Human mRNA for cytochrome b5, partial cds. |
| 577 | hu00825211 | g2662290 | Human mRNA for cytochrome b5, partial cds. |
| 578 | hu00470440 | g2662290 | Human mRNA for cytochrome b5, partial cds. |
| 579 | hu00174710 | g2662290 | Human mRNA for cytochrome b5, partial cds. |
| 580 | hu00049450 | g2662290 | Human mRNA for cytochrome b5, partial cds. |
| 581 | hu00516337 | g2662290 | Human mRNA for cytochrome b5, partial cds. |
| 582 | hu00779021 | g2662290 | Human mRNA for cytochrome b5, partial cds. |
| 583 | hu00870760 | g2662290 | Human mRNA for cytochrome b5, partial cds. |
| 584 | hu00507709 | g2662290 | Human mRNA for cytochrome b5, partial cds. |
| 585 | hu00826066 | g2662290 | Human mRNA for cytochrome b5, partial cds. |
| 586 | hu00526933 | g2662290 | Human mRNA for cytochrome b5, partial cds. |
| 587 | hu00634709 | g2662290 | Human mRNA for cytochrome b5, partial cds. |
| 588 | hu00279274 | g2662290 | Human mRNA for cytochrome b5, partial cds. |
| 589 | hu00046789 | g2662290 | Human mRNA for cytochrome b5, partial cds. |
| 590 | hu00686725 | g2662290 | Human mRNA for cytochrome b5, partial cds. |
| 591 | hu01211796 | g2443331 | Nfrl |
| 592 | hu01277569 | g2443331 | Nfrl |
| 593 | hu01227913 | g2443331 | Nfrl |
| 594 | hu00019002 | g3881842 | Similarity to E. coli 2-oxoglutarate dehydrogenase (SW:ODO1_ECOLI); cDNA EST |
| 595 | hu00074988 | g3881842 | Similarity to E. coli 2-oxoglutarate dehydrogenase (SW:ODO1_ECOLI); cDNA EST |
| 596 | hu00682203 | g3881842 | Similarity to E. coli 2-oxoglutarate dehydrogenase (SW:ODO1_ECOLI); cDNA EST |
| 597 | hu00405958 | g3881842 | Similarity to E. coli 2-oxoglutarate dehydrogenase (SW:ODO1_ECOLI); cDNA EST |
| 598 | hu00902417 | g3881842 | Similarity to E. coli 2-oxoglutarate dehydrogenase (SW:ODO1_ECOLI); cDNA EST |
| 599 | hu00882139 | g3881842 | Similarity to E. coli 2-oxoglutarate dehydrogenase (SW:ODO1_ECOLI); cDNA EST |
| 600 | hu00444939 | g3881842 | Similarity to E. coli 2-oxoglutarate dehydrogenase (SW:ODO1_ECOLI); cDNA EST |
| 601 | hu00996720 | g4375935 | dJ257I9.1 (similar to Cytochrome B) |
| 602 | hu00979613 | g4928472 | type 2 peroxiredoxin |
| 603 | hu01217988 | g4928472 | type 2 peroxiredoxin |
| 604 | hu01131267 | g4928472 | type 2 peroxiredoxin |
| 605 | hu01057108 | g4928472 | type 2 peroxiredoxin |
| 606 | hu01127156 | g4928472 | type 2 peroxiredoxin |
| 607 | hu01315257 | g4928472 | type 2 peroxiredoxin |
| 608 | hu01127950 | g4928472 | type 2 peroxiredoxin |
| 609 | hu01277533 | g4928472 | type 2 peroxiredoxin |
| 610 | hu01313696 | g4928472 | type 2 peroxiredoxin |
| 611 | hu01262379 | g4928472 | type 2 peroxiredoxin |
| 612 | hu01184935 | g4928472 | type 2 peroxiredoxin |
| 613 | hu01309738 | g4928472 | type 2 peroxiredoxin |
| 614 | hu01216150 | g4928472 | type 2 peroxiredoxin |
| 615 | hu01330055 | g4928472 | type 2 peroxiredoxin |
| 616 | hu01007235 | g4928472 | type 2 peroxiredoxin |
| 617 | hu00627285 | g3646127 | Human mRNA for putative thioredoxin-like protein. |
| 618 | rat00198924 | g2970690 | Mouse thioredoxin-related protein mRNA, complete cds. |
| 619 | rat00147149 | g2970690 | Mouse thioredoxin-related protein mRNA, complete cds. |
| 620 | rat00122728 | g2970690 | Mouse thioredoxin-related protein mRNA, complete cds. |
| 621 | rat00102041 | g2970690 | Mouse thioredoxin-related protein mRNA, complete cds. |
| 622 | rat00244239 | g2970690 | Mouse thioredoxin-related protein mRNA, complete cds. |
| 623 | hu00860841 | g3646127 | Human mRNA for putative thioredoxin-like protein. |
| 624 | hu00329532 | g3646127 | Human mRNA for putative thioredoxin-like protein. |
| 625 | hu00268088 | g3646127 | Human mRNA for putative thioredoxin-like protein. |
| 626 | hu00754801 | g3646127 | Human mRNA for putative thioredoxin-like protein. |
| 627 | rat00227438 | g2970690 | Mouse thioredoxin-related protein mRNA, complete cds. |
| 628 | rat00148580 | g2970690 | Mouse thioredoxin-related protein mRNA, complete cds. |
| 629 | rat00148626 | g2970690 | Mouse thioredoxin-related protein mRNA, complete cds. |
| 630 | rat00068841 | g2970690 | Mouse thioredoxin-related protein mRNA, complete cds. |
| 631 | rat00126954 | g2970690 | Mouse thioredoxin-related protein mRNA, complete cds. |
| 632 | rat00170875 | g2970690 | Mouse thioredoxin-related protein mRNA, complete cds. |
| 633 | rat00059770 | g2970690 | Mouse thioredoxin-related protein mRNA, complete cds. |
| 634 | rat00159473 | g2970690 | Mouse thioredoxin-related protein mRNA, complete cds. |
| 635 | rat00061995 | g2970690 | Mouse thioredoxin-related protein mRNA, complete cds. |
| 636 | rat00239188 | g2970690 | Mouse thioredoxin-related protein mRNA, complete cds. |
| 637 | rat00226688 | g2970690 | Mouse thioredoxin-related protein mRNA, complete cds. |
| 638 | rat00079387 | g2970690 | Mouse thioredoxin-related protein mRNA, complete cds. |
| 639 | rat00069857 | g2970690 | Mouse thioredoxin-related protein mRNA, complete cds. |
| 640 | rat00201032 | g2970690 | Mouse thioredoxin-related protein mRNA, complete cds. |
| 641 | rat00100124 | g2970690 | Mouse thioredoxin-related protein mRNA, complete cds. |
| 642 | rat00000065 | g2970690 | Mouse thioredoxin-related protein mRNA, complete cds. |
| 643 | rat00025121 | g2970690 | Mouse thioredoxin-related protein mRNA, complete cds. |
| 644 | rat00111342 | g2970690 | Mouse thioredoxin-related protein mRNA, complete cds. |
| 645 | rat00012470 | g2970690 | Mouse thioredoxin-related protein mRNA, complete cds. |
| 646 | rat00243312 | g2970690 | Mouse thioredoxin-related protein mRNA, complete cds. |
| 647 | rat00153024 | g2970690 | Mouse thioredoxin-related protein mRNA, complete cds. |
| 648 | rat00093202 | g2970690 | Mouse thioredoxin-related protein mRNA, complete cds. |
| 649 | rat00072036 | g2970690 | Mouse thioredoxin-related protein mRNA, complete cds. |
| 650 | rat00122347 | g2970690 | Mouse thioredoxin-related protein mRNA, complete cds. |
| 651 | rat00128414 | g2970690 | Mouse thioredoxin-related protein mRNA, complete cds. |
| 652 | rat00054660 | g2970690 | Mouse thioredoxin-related protein mRNA, complete cds. |
| 653 | rat00160662 | g2970690 | Mouse thioredoxin-related protein mRNA, complete cds. |
| 654 | rat00243365 | g2970690 | Mouse thioredoxin-related protein mRNA, complete cds. |
| 655 | hu00195965 | g3646127 | Human mRNA for putative thioredoxin-like protein. |
| 656 | hu00492988 | g3646127 | Human mRNA for putative thioredoxin-like protein. |
| 657 | hu00410537 | g3646127 | Human mRNA for putative thioredoxin-like protein. |
| 658 | hu00133553 | g3646127 | Human mRNA for putative thioredoxin-like protein. |
| 659 | hu00341716 | g3646127 | Human mRNA for putative thioredoxin-like protein. |
| 660 | hu00319153 | g3646127 | Human mRNA for putative thioredoxin-like protein. |
| 661 | hu00890410 | g3646127 | Human mRNA for putative thioredoxin-like protein. |
| 662 | hu00761631 | g3646127 | Human mRNA for putative thioredoxin-like protein. |
| 663 | hu00424618 | g3646127 | Human mRNA for putative thioredoxin-like protein. |
| 664 | hu00461663 | g3646127 | Human mRNA for putative thioredoxin-like protein. |
| 665 | hu00471664 | g3646127 | Human mRNA for putative thioredoxin-like protein. |
| 666 | hu00175737 | g3646127 | Human mRNA for putative thioredoxin-like protein. |
| 667 | hu00080697 | g3646127 | Human mRNA for putative thioredoxin-like protein. |
| 668 | hu00684950 | g3646127 | Human mRNA for putative thioredoxin-like protein. |
| 669 | hu00910769 | g3646127 | Human mRNA for putative thioredoxin-like protein. |
| 670 | hu00672773 | g3646127 | Human mRNA for putative thioredoxin-like protein. |
| 671 | hu00774949 | g3646127 | Human mRNA for putative thioredoxin-like protein. |
| 672 | hu00959976 | g3646127 | Human mRNA for putative thioredoxin-like protein. |
| 673 | hu00124133 | g3646127 | Human mRNA for putative thioredoxin-like protein. |
| 674 | hu00717302 | g3646127 | Human mRNA for putative thioredoxin-like protein. |
| 675 | hu00552353 | g3646127 | Human mRNA for putative thioredoxin-like protein. |
| 676 | hu00544172 | g3646127 | Human mRNA for putative thioredoxin-like protein. |
| 677 | hu00943161 | g3646127 | Human mRNA for putative thioredoxin-like protein. |
| 678 | hu00662183 | g3646127 | Human mRNA for putative thioredoxin-like protein. |
| 679 | hu00590931 | g3646127 | Human mRNA for putative thioredoxin-like protein. |
| 680 | hu00235972 | g3646127 | Human mRNA for putative thioredoxin-like protein. |
| 681 | hu00867135 | g3646127 | Human mRNA for putative thioredoxin-like protein. |
| 682 | hu00758833 | g3646127 | Human mRNA for putative thioredoxin-like protein. |
| 683 | hu00069795 | g3646127 | Human mRNA for putative thioredoxin-like protein. |
| 684 | hu00088668 | g3646127 | Human mRNA for putative thioredoxin-like protein. |
| 685 | hu00378757 | g3646127 | Human mRNA for putative thioredoxin-like protein. |
| 686 | hu00271021 | g3646127 | Human mRNA for putative thioredoxin-like protein. |
| 687 | hu00238010 | g3646127 | Human mRNA for putative thioredoxin-like protein. |
| 688 | hu00241262 | g3646127 | Human mRNA for putative thioredoxin-like protein. |
| 689 | hu00000477 | g3646127 | Human mRNA for putative thioredoxin-like protein. |
| 690 | hu00432335 | g3646127 | Human mRNA for putative thioredoxin-like protein. |
| 691 | hu00015563 | g3646127 | Human mRNA for putative thioredoxin-like protein. |
| 692 | hu00646150 | g3646127 | Human mRNA for putative thioredoxin-like protein. |
| 693 | hu00062618 | g3646127 | Human mRNA for putative thioredoxin-like protein. |
| 694 | hu00091760 | g3646127 | Human mRNA for putative thioredoxin-like protein. |
| 695 | hu00466331 | g3646127 | Human mRNA for putative thioredoxin-like protein. |
| 696 | hu00624371 | g3646127 | Human mRNA for putative thioredoxin-like protein. |
| 697 | hu00613446 | g3646127 | Human mRNA for putative thioredoxin-like protein. |
| 698 | hu00107379 | g3646127 | Human mRNA for putative thioredoxin-like protein. |
| 699 | hu00736344 | g3646127 | Human mRNA for putative thioredoxin-like protein. |
| 700 | hu00049527 | g3646127 | Human mRNA for putative thioredoxin-like protein. |
| 701 | hu00123894 | g3646127 | Human mRNA for putative thioredoxin-like protein. |
| 702 | hu00362585 | g3646127 | Human mRNA for putative thioredoxin-like protein. |
| 703 | hu00772940 | g3646127 | Human mRNA for putative thioredoxin-like protein. |
| 704 | hu00567651 | g3646127 | Human mRNA for putative thioredoxin-like protein. |
| 705 | hu00884551 | g3646127 | Human mRNA for putative thioredoxin-like protein. |
| 706 | hu00217968 | g3646127 | Human mRNA for putative thioredoxin-like protein. |
| 707 | hu00653686 | g3646127 | Human mRNA for putative thioredoxin-like protein. |
| 708 | hu00666277 | g3646127 | Human mRNA for putative thioredoxin-like protein. |
| 709 | hu00682873 | g3646127 | Human mRNA for putative thioredoxin-like protein. |
| 710 | hu00901002 | g3646127 | Human mRNA for putative thioredoxin-like protein. |
| 711 | hu00712113 | g3646127 | Human mRNA for putative thioredoxin-like protein. |
| 712 | hu00090046 | g3646127 | Human mRNA for putative thioredoxin-like protein. |
| 713 | hu00016543 | g3646127 | Human mRNA for putative thioredoxin-like protein. |
| 714 | hu00102935 | g3646127 | Human mRNA for putative thioredoxin-like protein. |
| 715 | hu00170391 | g3646127 | Human mRNA for putative thioredoxin-like protein. |
| 716 | hu00291433 | g3646127 | Human mRNA for putative thioredoxin-like protein. |
| 717 | hu00433914 | g3646127 | Human mRNA for putative thioredoxin-like protein. |
| 718 | hu00379104 | g3646127 | Human mRNA for putative thioredoxin-like protein. |
| 719 | hu00622960 | g3646127 | Human mRNA for putative thioredoxin-like protein. |
| 720 | hu00316357 | g3646127 | Human mRNA for putative thioredoxin-like protein. |
| 721 | hu00352638 | g3646127 | Human mRNA for putative thioredoxin-like protein. |
| 722 | hu00542377 | g3646127 | Human mRNA for putative thioredoxin-like protein. |
| 723 | hu00484526 | g3646127 | Human mRNA for putative thioredoxin-like protein. |
| 724 | hu00962138 | g3646127 | Human mRNA for putative thioredoxin-like protein. |
| 725 | hu00961725 | g3646127 | Human mRNA for putative thioredoxin-like protein. |
| 726 | hu00545448 | g3646127 | Human mRNA for putative thioredoxin-like protein. |
| 727 | hu00646249 | g3646127 | Human mRNA for putative thioredoxin-like protein. |
| 728 | hu00337964 | g3646127 | Human mRNA for putative thioredoxin-like protein. |
| 729 | hu00854449 | g3646127 | Human mRNA for putative thioredoxin-like protein. |
| 730 | hu01258892 | g2897941 | Human thioredoxin-like protein mRNA, complete cds. |
| 731 | hu01068980 | g2897941 | Human thioredoxin-like protein mRNA, complete cds. |
| 732 | rat00013687 | g203718 | Rat (Sprague-Dawley) cytochrome c pseudogene, clone Ch4A-RC9. |
| 733 | rat00197623 | g203718 | Rat (Sprague-Dawley) cytochrome c pseudogene, clone Ch4A-RC9. |
| 734 | rat00079184 | g203718 | Rat (Sprague-Dawley) cytochrome c pseudogene, clone Ch4A-RC9. |
| 735 | rat00097770 | g203718 | Rat (Sprague-Dawley) cytochrome c pseudogene, clone Ch4A-RC9. |
| 736 | rat00109965 | g203718 | Rat (Sprague-Dawley) cytochrome c pseudogene, clone Ch4A-RC9. |
| 737 | rat00104653 | g203718 | Rat (Sprague-Dawley) cytochrome c pseudogene, clone Ch4A-RC9. |
| 738 | rat00142167 | g203718 | Rat (Sprague-Dawley) cytochrome c pseudogene, clone Ch4A-RC9. |
| 739 | rat00259341 | g203718 | Rat (Sprague-Dawley) cytochrome c pseudogene, clone Ch4A-RC9. |
| 740 | hu00601827 | g453963 | Human mRNA for ATL-derived factor/thiredoxin. |
| 741 | hu00024989 | g453963 | Human mRNA for ATL-derived factor/thiredoxin. |
| 742 | hu00999345 | g2897941 | Human thioredoxin-like protein mRNA, complete cds. |
| 743 | hu01011601 | g2897941 | Human thioredoxin-like protein mRNA, complete cds. |
| 744 | hu01143411 | g2897941 | Human thioredoxin-like protein mRNA, complete cds. |
| 745 | hu01122354 | g2897941 | Human thioredoxin-like protein mRNA, complete cds. |
| 746 | hu01215505 | g2897941 | Human thioredoxin-like protein mRNA, complete cds. |
| 747 | hu01004563 | g2897941 | Human thioredoxin-like protein mRNA, complete cds. |
| 748 | hu01075341 | g2897941 | Human thioredoxin-like protein mRNA, complete cds. |
| 749 | hu01271901 | g2897941 | Human thioredoxin-like protein mRNA, complete cds. |
| 750 | hu01189121 | g2897941 | Human thioredoxin-like protein mRNA, complete cds. |
| 751 | hu01263266 | g2897941 | Human thioredoxin-like protein mRNA, complete cds. |
| 752 | hu01258550 | g2897941 | Human thioredoxin-like protein mRNA, complete cds. |
| 753 | hu01091807 | g2897941 | Human thioredoxin-like protein mRNA, complete cds. |
| 754 | hu01325277 | g2897941 | Human thioredoxin-like protein mRNA, complete cds. |
| 755 | hu01072756 | g2897941 | Human thioredoxin-like protein mRNA, complete cds. |
| 756 | hu01305668 | g2897941 | Human thioredoxin-like protein mRNA, complete cds. |
| 757 | hu01034966 | g2897941 | Human thioredoxin-like protein mRNA, complete cds. |
| 758 | hu01268635 | g2897941 | Human thioredoxin-like protein mRNA, complete cds. |
| 759 | hu00967441 | g2897941 | Human thioredoxin-like protein mRNA, complete cds. |
| 760 | hu01185674 | g2897941 | Human thioredoxin-like protein mRNA, complete cds. |
| 761 | hu01089257 | g2897941 | Human thioredoxin-like protein mRNA, complete cds. |
| 762 | hu01070889 | g2897941 | Human thioredoxin-like protein mRNA, complete cds. |
| 763 | hu00396958 | g453963 | Human mRNA for ATL-derived factor/thiredoxin. |
| 764 | hu00230202 | g453963 | Human mRNA for ATL-derived factor/thiredoxin. |
| 765 | hu00300233 | g453963 | Human mRNA for ATL-derived factor/thiredoxin. |
| 766 | hu00433221 | g453963 | Human mRNA for ATL-derived factor/thiredoxin. |
| 767 | hu00150706 | g453963 | Human mRNA for ATL-derived factor/thiredoxin. |
| 768 | hu00270623 | g453963 | Human mRNA for ATL-derived factor/thiredoxin. |
| 769 | hu00119128 | g453963 | Human mRNA for ATL-derived factor/thiredoxin. |
| 770 | hu00608710 | g453963 | Human mRNA for ATL-derived factor/thiredoxin. |
| 771 | hu00804308 | g453963 | Human mRNA for ATL-derived factor/thiredoxin. |
| 772 | hu00284711 | g453963 | Human mRNA for ATL-derived factor/thiredoxin. |
| 773 | hu00490282 | g453963 | Human mRNA for ATL-derived factor/thiredoxin. |
| 774 | hu00139714 | g453963 | Human mRNA for ATL-derived factor/thiredoxin. |
| 775 | hu00231892 | g453963 | Human mRNA for ATL-derived factor/thiredoxin. |
| 776 | hu00357416 | g453963 | Human mRNA for ATL-derived factor/thiredoxin. |
| 777 | hu00515780 | g453963 | Human mRNA for ATL-derived factor/thiredoxin. |
| 778 | hu00620289 | g453963 | Human mRNA for ATL-derived factor/thiredoxin. |
| 779 | hu00634536 | g453963 | Human mRNA for ATL-derived factor/thiredoxin. |
| 780 | hu00295381 | g453963 | Human mRNA for ATL-derived factor/thiredoxin. |
| 781 | hu00689090 | g453963 | Human mRNA for ATL-derived factor/thiredoxin. |
| 782 | hu00624894 | g453963 | Human mRNA for ATL-derived factor/thiredoxin. |
| 783 | rat00180808 | g3688520 | Rat mRNA for thiol-specific antioxidant protein (1-Cys peroxiredoxin). |
| 784 | rat00029077 | g3688520 | Rat mRNA for thiol-specific antioxidant protein (1-Cys peroxiredoxin). |
| 785 | rat00238831 | g3688520 | Rat mRNA for thiol-specific antioxidant protein (1-Cys peroxiredoxin). |
| 786 | rat00157148 | g3688520 | Rat mRNA for thiol-specific antioxidant protein (1-Cys peroxiredoxin). |
| 787 | rat00228376 | g3688520 | Rat mRNA for thiol-specific antioxidant protein (1-Cys peroxiredoxin). |
| 788 | rat00112998 | g3688520 | Rat mRNA for thiol-specific antioxidant protein (1-Cys peroxiredoxin). |
| 789 | rat00067297 | g3688520 | Rat mRNA for thiol-specific antioxidant protein (1-Cys peroxiredoxin). |
| 790 | rat00099177 | g3688520 | Rat mRNA for thiol-specific antioxidant protein (1-Cys peroxiredoxin). |
| 791 | rat00188749 | g3688520 | Rat mRNA for thiol-specific antioxidant protein (1-Cys peroxiredoxin). |
| 792 | rat00149897 | g3688520 | Rat mRNA for thiol-specific antioxidant protein (1-Cys peroxiredoxin). |
| 793 | rat00102720 | g3688520 | Rat mRNA for thiol-specific antioxidant protein (1-Cys peroxiredoxin). |
| 794 | rat00119114 | g3688520 | Rat mRNA for thiol-specific antioxidant protein (1-Cys peroxiredoxin). |
| 795 | rat00214272 | g3688520 | Rat mRNA for thiol-specific antioxidant protein (1-Cys peroxiredoxin). |
| 796 | rat00166231 | g3688520 | Rat mRNA for thiol-specific antioxidant protein (1-Cys peroxiredoxin). |
| 797 | rat00071074 | g3688520 | Rat mRNA for thiol-specific antioxidant protein (1-Cys peroxiredoxin). |
| 798 | rat00240806 | g3688520 | Rat mRNA for thiol-specific antioxidant protein (1-Cys peroxiredoxin). |
| 799 | rat00017707 | g3688520 | Rat mRNA for thiol-specific antioxidant protein (1-Cys peroxiredoxin). |
| 800 | rat00103279 | g3688520 | Rat mRNA for thiol-specific antioxidant protein (1-Cys peroxiredoxin). |
| 801 | rat00179213 | g3688520 | Rat mRNA for thiol-specific antioxidant protein (1-Cys peroxiredoxin). |
| 802 | rat00155382 | g3688520 | Rat mRNA for thiol-specific antioxidant protein (1-Cys peroxiredoxin). |
| 803 | rat00241944 | g3688520 | Rat mRNA for thiol-specific antioxidant protein (1-Cys peroxiredoxin). |
| 804 | hu00788751 | g2911066 | adrenodoxin-like protein |
| 805 | hu00263898 | g2911066 | adrenodoxin-like protein |
| 806 | hu00679088 | g2911066 | adrenodoxin-like protein |
| 807 | hu00411401 | g2911066 | adrenodoxin-like protein |
| 808 | hu00849869 | g2911066 | adrenodoxin-like protein |
| 809 | hu00733110 | g2911066 | adrenodoxin-like protein |
| 810 | hu00500094 | g2911066 | adrenodoxin-like protein |
| 811 | hu00630613 | g2911066 | adrenodoxin-like protein |
| 812 | hu00483211 | g2911066 | adrenodoxin-like protein |
| 813 | hu00726024 | g2911066 | adrenodoxin-like protein |
| 814 | hu00804640 | g2911066 | adrenodoxin-like protein |
| 815 | hu00434840 | g2911066 | adrenodoxin-like protein |
| 816 | hu00307931 | g2911066 | adrenodoxin-like protein |
| 817 | hu00118249 | g2911066 | adrenodoxin-like protein |
| 818 | hu00359170 | g2911066 | adrenodoxin-like protein |
| 819 | hu00834424 | g2911066 | adrenodoxin-like protein |
| 820 | hu00491309 | g2911066 | adrenodoxin-like protein |
| 821 | hu00641394 | g2911066 | adrenodoxin-like protein |
| 822 | hu00529683 | g2911066 | adrenodoxin-like protein |
| 823 | hu00237499 | g2911066 | adrenodoxin-like protein |
| 824 | hu00697147 | g2911066 | adrenodoxin-like protein |
| 825 | hu00197335 | g2443331 | Nfrl |
| 826 | hu00731987 | g5295993 | Human SDHD gene for small subunit of cytochrome b of succinate dehydrogenase, complete cds. |
| 827 | hu00795629 | g5295993 | Human SDHD gene for small subunit of cytochrome b of succinate dehydrogenase, complete cds. |
| 828 | hu00885639 | g5295993 | Human SDHD gene for small subunit of cytochrome b of succinate dehydrogenase, complete cds. |
| 829 | hu00728978 | g5295993 | Human SDHD gene for small subunit of cytochrome b of succinate dehydrogenase, complete cds. |
| 830 | hu00531230 | g5295993 | Human SDHD gene for small subunit of cytochrome b of succinate dehydrogenase, complete cds. |
| 831 | hu00502701 | g5295993 | Human SDHD gene for small subunit of cytochrome b of succinate dehydrogenase, complete cds. |
| 832 | hu00125531 | g5295993 | Human SDHD gene for small subunit of cytochrome b of succinate dehydrogenase, complete cds. |
| 833 | hu00161830 | g5295993 | Human SDHD gene for small subunit of cytochrome b of succinate dehydrogenase, complete cds. |
| 834 | hu00113816 | g5295993 | Human SDHD gene for small subunit of cytochrome b of succinate dehydrogenase, complete cds. |
| 835 | hu00763211 | g5295993 | Human SDHD gene for small subunit of cytochrome b of succinate dehydrogenase, complete cds. |
| 836 | hu00623901 | g5295993 | Human SDHD gene for small subunit of cytochrome b of succinate dehydrogenase, complete cds. |
| 837 | hu00447421 | g5295993 | Human SDHD gene for small subunit of cytochrome b of succinate dehydrogenase, complete cds. |
| 838 | hu00550627 | g5295993 | Human SDHD gene for small subunit of cytochrome b of succinate dehydrogenase, complete cds. |
| 839 | hu00740186 | g5295993 | Human SDHD gene for small subunit of cytochrome b of succinate dehydrogenase, complete cds. |
| 840 | hu00440838 | g5295993 | Human SDHD gene for small subunit of cytochrome b of succinate dehydrogenase, complete cds. |
| 841 | hu00223687 | g5295993 | Human SDHD gene for small subunit of cytochrome b of succinate dehydrogenase, complete cds. |
| 842 | hu00872394 | g5295993 | Human SDHD gene for small subunit of cytochrome b of succinate dehydrogenase, complete cds. |
| 843 | hu00834378 | g5295993 | Human SDHD gene for small subunit of cytochrome b of succinate dehydrogenase, complete cds. |
| 844 | hu00554569 | g5295993 | Human SDHD gene for small subunit of cytochrome b of succinate dehydrogenase, complete cds. |
| 845 | hu00743996 | g5295993 | Human SDHD gene for small subunit of cytochrome b of succinate dehydrogenase, complete cds. |
| 846 | hu00919779 | g5295993 | Human SDHD gene for small subunit of cytochrome b of succinate dehydrogenase, complete cds. |
| 847 | hu00107621 | g5295993 | Human SDHD gene for small subunit of cytochrome b of succinate dehydrogenase, complete cds. |
| 848 | hu00321704 | g5295993 | Human SDHD gene for small subunit of cytochrome b of succinate dehydrogenase, complete cds. |
| 849 | hu00104670 | g5295993 | Human SDHD gene for small subunit of cytochrome b of succinate dehydrogenase, complete cds. |
| 850 | hu00102690 | g5295993 | Human SDHD gene for small subunit of cytochrome b of succinate dehydrogenase, complete cds. |
| 851 | hu00527304 | g5295993 | Human SDHD gene for small subunit of cytochrome b of succinate dehydrogenase, complete cds. |
| 852 | hu00017059 | g5295993 | Human SDHD gene for small subunit of cytochrome b of succinate dehydrogenase, complete cds. |
| 853 | hu00850406 | g5295993 | Human SDHD gene for small subunit of cytochrome b of succinate dehydrogenase, complete cds. |
| 854 | hu00793203 | g5295993 | Human SDHD gene for small subunit of cytochrome b of succinate dehydrogenase, complete cds. |
| 855 | hu00732715 | g5295993 | Human SDHD gene for small subunit of cytochrome b of succinate dehydrogenase, complete cds. |
| 856 | hu00242629 | g5295993 | Human SDHD gene for small subunit of cytochrome b of succinate dehydrogenase, complete cds. |
| 857 | hu00591902 | g5295993 | Human SDHD gene for small subunit of cytochrome b of succinate dehydrogenase, complete cds. |
| 858 | hu00611478 | g5295993 | Human SDHD gene for small subunit of cytochrome b of succinate dehydrogenase, complete cds. |
| 859 | hu00514953 | g5295993 | Human SDHD gene for small subunit of cytochrome b of succinate dehydrogenase, complete cds. |
| 860 | hu00847134 | g5295993 | Human SDHD gene for small subunit of cytochrome b of succinate dehydrogenase, complete cds. |
| 861 | hu00527429 | g5295993 | Human SDHD gene for small subunit of cytochrome b of succinate dehydrogenase, complete cds. |
| 862 | hu00778152 | g5295993 | Human SDHD gene for small subunit of cytochrome b of succinate dehydrogenase, complete cds. |
| 863 | hu00483538 | g5295993 | Human SDHD gene for small subunit of cytochrome b of succinate dehydrogenase, complete cds. |
| 864 | hu00169762 | g5295993 | Human SDHD gene for small subunit of cytochrome b of succinate dehydrogenase, complete cds. |
| 865 | hu00627494 | g5295993 | Human SDHD gene for small subunit of cytochrome b of succinate dehydrogenase, complete cds. |
| 866 | hu00683672 | g5295993 | Human SDHD gene for small subunit of cytochrome b of succinate dehydrogenase, complete cds. |
| 867 | hu00619358 | g5295993 | Human SDHD gene for small subunit of cytochrome b of succinate dehydrogenase, complete cds. |
| 868 | hu00334846 | g2443331 | Nfrl |
| 869 | hu00538435 | g2443331 | Nfrl |
| 870 | hu00045975 | g2443331 | Nfrl |
| 871 | hu00255713 | g2443331 | Nfrl |
| 872 | hu00309830 | g2443331 | Nfrl |
| 873 | hu00838320 | g2443331 | Nfrl |
| 874 | hu00600416 | g2443331 | Nfrl |
| 875 | hu00678814 | g2443331 | Nfrl |
| 876 | hu00220365 | g2443331 | Nfrl |
| 877 | hu00715266 | g2911066 | adrenodoxin-like protein |
| 878 | hu00061515 | g2911066 | adrenodoxin-like protein |
| 879 | hu01256988 | g37118 | Human gene for thioredoxin, exons 2 and 3. |
| 880 | hu01320806 | g181261 | Human somatic cytochrome c (HC6) processed pseudogene, complete cds. |
| 881 | hu00225272 | g2351036 | Human mRNA for cytochrome b small subunit of complex II, complete cds. |
| 882 | hu00492301 | g4375935 | dJ257I9.1 (similar to Cytochrome B) |
| 883 | hu00147708 | g4375935 | dJ257I9.1 (similar to Cytochrome B) |
| 884 | hu00720528 | g4375935 | dJ257I9.1 (similar to Cytochrome B) |
| 885 | hu00662648 | g4375935 | dJ257I9.1 (similar to Cytochrome B) |
| 886 | hu00425257 | g4375935 | dJ257I9.1 (similar to Cytochrome B) |
| 887 | hu01145798 | g2662290 | Human mRNA for cytochrome b5, partial cds. |
| 888 | hu00990657 | g2662290 | Human mRNA for cytochrome b5, partial cds. |
| 889 | hu01033267 | g2662290 | Human mRNA for cytochrome b5, partial cds. |
| 890 | hu01064572 | g2662290 | Human mRNA for cytochrome b5, partial cds. |
| 891 | hu01014659 | g2662290 | Human mRNA for cytochrome b5, partial cds. |
| 892 | hu01343943 | g2662290 | Human mRNA for cytochrome b5, partial cds. |
| 893 | hu01172385 | g2662290 | Human mRNA for cytochrome b5, partial cds. |
| 894 | hu01274383 | g2662290 | Human mRNA for cytochrome b5, partial cds. |
| 895 | hu01126099 | g2662290 | Human mRNA for cytochrome b5, partial cds. |
| 896 | hu01096453 | g2662290 | Human mRNA for cytochrome b5, partial cds. |
| 897 | hu01280694 | g2662290 | Human mRNA for cytochrome b5, partial cds. |
| 898 | hu01303215 | g2662290 | Human mRNA for cytochrome b5, partial cds. |
| 899 | hu01082041 | g2662290 | Human mRNA for cytochrome b5, partial cds. |
| 900 | hu00153508 | g2967825 | Human thioredoxin homolog mRNA, complete cds. |
| 901 | hu00928636 | g2967825 | Human thioredoxin homolog mRNA, complete cds. |
| 902 | hu00498907 | g2897941 | Human thioredoxin-like protein mRNA, complete cds. |
| 903 | hu01128530 | g897580 | Human iron-regulatory protein 2 (IRP2) mRNA, partial cds. |
| 904 | hu01111791 | g897580 | Human iron-regulatory protein 2 (IRP2) mRNA, partial cds. |
| 905 | hu01274800 | g897580 | Human iron-regulatory protein 2 (IRP2) mRNA, partial cds. |
| 906 | hu01075270 | g897580 | Human iron-regulatory protein 2 (IRP2) mRNA, partial cds. |
| 907 | hu01040299 | g897580 | Human iron-regulatory protein 2 (IRP2) mRNA, partial cds. |
| 908 | hu01234846 | g897580 | Human iron-regulatory protein 2 (IRP2) mRNA, partial cds. |
| 909 | hu01296534 | g897580 | Human iron-regulatory protein 2 (IRP2) mRNA, partial cds. |
| 910 | hu01176871 | g897580 | Human iron-regulatory protein 2 (IRP2) mRNA, partial cds. |
| 911 | hu00970376 | g897580 | Human iron-regulatory protein 2 (IRP2) mRNA, partial cds. |
| 912 | hu00188328 | g2967825 | Human thioredoxin homolog mRNA, complete cds. |
| 913 | hu00512707 | g2967825 | Human thioredoxin homolog mRNA, complete cds. |
| 914 | hu00458446 | g2967825 | Human thioredoxin homolog mRNA, complete cds. |
| 915 | hu00310776 | g2967825 | Human thioredoxin homolog mRNA, complete cds. |
| 916 | hu00878881 | g2967825 | Human thioredoxin homolog mRNA, complete cds. |
| 917 | hu00695058 | g2967825 | Human thioredoxin homolog mRNA, complete cds. |
| 918 | hu00809565 | g2967825 | Human thioredoxin homolog mRNA, complete cds. |
| 919 | hu00422460 | g2967825 | Human thioredoxin homolog mRNA, complete cds. |
| 920 | hu00236474 | g2967825 | Human thioredoxin homolog mRNA, complete cds. |
| 921 | hu00504324 | g2967825 | Human thioredoxin homolog mRNA, complete cds. |
| 922 | hu00665846 | g4500377 | putative NADPH cytochrome reductase |
| 923 | hu00369297 | g4500377 | putative NADPH cytochrome reductase |
| 924 | hu00729723 | g4500377 | putative NADPH cytochrome reductase |
| 925 | hu00228581 | g4500377 | putative NADPH cytochrome reductase |
| 926 | hu00747970 | g4500377 | putative NADPH cytochrome reductase |
| 927 | rat00217853 | g786396 | cytochrome bc-1 complex core P {clone E518, estrogen induced gene} [rats, Sprague-Dawley, |
| 928 | rat00105569 | g786396 | cytochrome bc-1 complex core P {clone E518, estrogen induced gene} [rats, Sprague-Dawley, |
| 929 | rat00066626 | g786396 | cytochrome bc-1 complex core P {clone E518, estrogen induced gene} [rats, Sprague-Dawley, |
| 930 | rat00209690 | g786396 | cytochrome bc-1 complex core P {clone E518, estrogen induced gene} [rats, Sprague-Dawley, |
| 931 | rat00193805 | g786396 | cytochrome bc-1 complex core P {clone E518, estrogen induced gene} [rats, Sprague-Dawley, |
| 932 | rat00080783 | g786396 | cytochrome bc-1 complex core P {clone E518, estrogen induced gene} [rats, Sprague-Dawley, |
| 933 | rat00177525 | g786396 | cytochrome bc-1 complex core P {clone E518, estrogen induced gene} [rats, Sprague-Dawley, |
| 934 | rat00068166 | g786396 | cytochrome bc-1 complex core P {clone E518, estrogen induced gene} [rats, Sprague-Dawley, |
| 935 | rat00129844 | g786396 | cytochrome bc-1 complex core P {clone E518, estrogen induced gene} [rats, Sprague-Dawley, |
| 936 | rat00159187 | g786396 | cytochrome bc-1 complex core P {clone E518, estrogen induced gene} [rats, Sprague-Dawley, |
| 937 | rat00126977 | g786396 | cytochrome bc-1 complex core P {clone E518, estrogen induced gene} [rats, Sprague-Dawley, |
| 938 | rat00085597 | g786396 | cytochrome bc-1 complex core P {clone E518, estrogen induced gene} [rats, Sprague-Dawley, |
| 939 | rat00002212 | g786396 | cytochrome bc-1 complex core P {clone E518, estrogen induced gene} [rats, Sprague-Dawley, |
| 940 | rat00240035 | g786396 | cytochrome bc-1 complex core P {clone E518, estrogen induced gene} [rats, Sprague-Dawley, |
| 941 | rat00004910 | g786396 | cytochrome bc-1 complex core P {clone E518, estrogen induced gene} [rats, Sprague-Dawley, |
| 942 | rat00150261 | g786396 | cytochrome bc-1 complex core P {clone E518, estrogen induced gene} [rats, Sprague-Dawley, |
| 943 | rat00106163 | g786396 | cytochrome bc-1 complex core P {clone E518, estrogen induced gene} [rats, Sprague-Dawley, |
| 944 | rat00035083 | g786396 | cytochrome bc-1 complex core P {clone E518, estrogen induced gene} [rats, Sprague-Dawley, |
| 945 | rat00203069 | g786396 | cytochrome bc-1 complex core P {clone E518, estrogen induced gene} [rats, Sprague-Dawley, |
| 946 | rat00028253 | g786396 | cytochrome bc-1 complex core P {clone E518, estrogen induced gene} [rats, Sprague-Dawley, |
| 947 | rat00078430 | g786396 | cytochrome bc-1 complex core P {clone E518, estrogen induced gene} [rats, Sprague-Dawley, |
| 948 | rat00056155 | g786396 | cytochrome bc-1 complex core P {clone E518, estrogen induced gene} [rats, Sprague-Dawley, |
| 949 | rat00104549 | g786396 | cytochrome bc-1 complex core P {clone E518, estrogen induced gene} [rats, Sprague-Dawley, |
| 950 | rat00108680 | g786396 | cytochrome bc-1 complex core P {clone E518, estrogen induced gene} [rats, Sprague-Dawley, |
| 951 | rat00127454 | g786396 | cytochrome bc-1 complex core P {clone E518, estrogen induced gene} [rats, Sprague-Dawley, |
| 952 | rat00081843 | g786396 | cytochrome bc-1 complex core P {clone E518, estrogen induced gene} [rats, Sprague-Dawley, |
| 953 | rat00137590 | g786396 | cytochrome bc-1 complex core P {clone E518, estrogen induced gene} [rats, Sprague-Dawley, |
| 954 | rat00190565 | g786396 | cytochrome bc-1 complex core P {clone E518, estrogen induced gene} [rats, Sprague-Dawley, |
| 955 | rat00013868 | g786396 | cytochrome bc-1 complex core P {clone E518, estrogen induced gene} [rats, Sprague-Dawley, |
| 956 | rat00162269 | g786396 | cytochrome bc-1 complex core P {clone E518, estrogen induced gene} [rats, Sprague-Dawley, |
| 957 | rat00088471 | g786396 | cytochrome bc-1 complex core P {clone E518, estrogen induced gene} [rats, Sprague-Dawley, |
| 958 | rat00093566 | g786396 | cytochrome bc-1 complex core P {clone E518, estrogen induced gene} [rats, Sprague-Dawley, |
| 959 | rat00042779 | g786396 | cytochrome bc-1 complex core P {clone E518, estrogen induced gene} [rats, Sprague-Dawley, |
| 960 | rat00017885 | g786396 | cytochrome bc-1 complex core P {clone E518, estrogen induced gene} [rats, Sprague-Dawley, |
| 961 | rat00171598 | g786396 | cytochrome bc-1 complex core P {clone E518, estrogen induced gene} [rats, Sprague-Dawley, |
| 962 | rat00189715 | g786396 | cytochrome bc-1 complex core P {clone E518, estrogen induced gene} [rats, Sprague-Dawley, |
| 963 | rat00053789 | g786396 | cytochrome bc-1 complex core P {clone E518, estrogen induced gene} [rats, Sprague-Dawley, |
| 964 | rat00175856 | g786396 | cytochrome bc-1 complex core P {clone E518, estrogen induced gene} [rats, Sprague-Dawley, |
| 965 | rat00164967 | g786396 | cytochrome bc-1 complex core P {clone E518, estrogen induced gene} [rats, Sprague-Dawley, |
| 966 | rat00078693 | g786396 | cytochrome bc-1 complex core P {clone E518, estrogen induced gene} [rats, Sprague-Dawley, |
| 967 | rat00066877 | g786396 | cytochrome bc-1 complex core P {clone E518, estrogen induced gene} [rats, Sprague-Dawley, |
| 968 | rat00096796 | g786396 | cytochrome bc-1 complex core P {clone E518, estrogen induced gene} [rats, Sprague-Dawley, |
| 969 | rat00161402 | g786396 | cytochrome bc-1 complex core P {clone E518, estrogen induced gene} [rats, Sprague-Dawley, |
| 970 | rat00161602 | g786396 | cytochrome bc-1 complex core P {clone E518, estrogen induced gene} [rats, Sprague-Dawley, |
| 971 | rat00010529 | g786396 | cytochrome bc-1 complex core P {clone E518, estrogen induced gene} [rats, Sprague-Dawley, |
| 972 | rat00020781 | g786396 | cytochrome bc-1 complex core P {clone E518, estrogen induced gene} [rats, Sprague-Dawley, |
| 973 | rat00100296 | g786396 | cytochrome bc-1 complex core P {clone E518, estrogen induced gene} [rats, Sprague-Dawley, |
| 974 | rat00185004 | g786396 | cytochrome bc-1 complex core P {clone E518, estrogen induced gene} [rats, Sprague-Dawley, |
| 975 | rat00024363 | g786396 | cytochrome bc-1 complex core P {clone E518, estrogen induced gene} [rats, Sprague-Dawley, |
| 976 | rat00211395 | g786396 | cytochrome bc-1 complex core P {clone E518, estrogen induced gene} [rats, Sprague-Dawley, |
| 977 | rat00061195 | g786396 | cytochrome bc-1 complex core P {clone E518, estrogen induced gene} [rats, Sprague-Dawley, |
| 978 | rat00003893 | g786396 | cytochrome bc-1 complex core P {clone E518, estrogen induced gene} [rats, Sprague-Dawley, |
| 979 | rat00184074 | g786396 | cytochrome bc-1 complex core P {clone E518, estrogen induced gene} [rats, Sprague-Dawley, |
| 980 | rat00028608 | g786396 | cytochrome bc-1 complex core P {clone E518, estrogen induced gene} [rats, Sprague-Dawley, |
| 981 | rat00050865 | g786396 | cytochrome bc-1 complex core P {clone E518, estrogen induced gene} [rats, Sprague-Dawley, |
| 982 | rat00142197 | g786396 | cytochrome bc-1 complex core P {clone E518, estrogen induced gene} [rats, Sprague-Dawley, |
| 983 | hu00969530 | g181228 | Human cytochrome b5 mRNA, 3′ end. |
| 984 | rat00178844 | g50622 | Mouse cytochrome c pseudogene (MC2). |
| 985 | hu00523841 | g5114277 | iron-sulfur protein |
| 986 | hu00759605 | g5114277 | iron-sulfur protein |
| 987 | rat00161197 | g57385 | Rat mRNA for thioredoxin. |
| 988 | rat00214410 | g57385 | Rat mRNA for thioredoxin. |
| 989 | rat00016800 | g57385 | Rat mRNA for thioredoxin. |
| 990 | rat00113622 | g57385 | Rat mRNA for thioredoxin. |
| 991 | rat00090205 | g57385 | Rat mRNA for thioredoxin. |
| 992 | rat00165130 | g57385 | Rat mRNA for thioredoxin. |
| 993 | rat00019997 | g57385 | Rat mRNA for thioredoxin. |
| 994 | rat00171632 | g57385 | Rat mRNA for thioredoxin. |
| 995 | rat00188498 | g57385 | Rat mRNA for thioredoxin. |
| 996 | rat00044347 | g57385 | Rat mRNA for thioredoxin. |
| 997 | rat00012121 | g57385 | Rat mRNA for thioredoxin. |
| 998 | rat00249136 | g3646128 | thioredoxin-like protein |
| 999 | rat00192355 | g3646128 | thioredoxin-like protein |
| 1000 | rat00227393 | g3646128 | thioredoxin-like protein |
| 1001 | rat00057555 | g3646128 | thioredoxin-like protein |
| 1002 | rat00085573 | g3646128 | thioredoxin-like protein |
| 1003 | rat00194473 | g3646128 | thioredoxin-like protein |
| 1004 | rat00193088 | g3646128 | thioredoxin-like protein |
| 1005 | rat00029775 | g3646128 | thioredoxin-like protein |
| 1006 | rat00177882 | g3646128 | thioredoxin-like protein |
| 1007 | rat00219996 | g3646128 | thioredoxin-like protein |
| 1008 | rat00233601 | g3646128 | thioredoxin-like protein |
| 1009 | rat00023406 | g3646128 | thioredoxin-like protein |
| 1010 | rat00111931 | g3646128 | thioredoxin-like protein |
| 1011 | rat00107350 | g3646128 | thioredoxin-like protein |
| 1012 | rat00142146 | g3646128 | thioredoxin-like protein |
| 1013 | rat00211383 | g3646128 | thioredoxin-like protein |
| 1014 | rat00044435 | g3646128 | thioredoxin-like protein |
| 1015 | rat00021863 | g3646128 | thioredoxin-like protein |
| 1016 | rat00230438 | g3646128 | thioredoxin-like protein |
| 1017 | rat00184305 | g3646128 | thioredoxin-like protein |
| 1018 | rat00160545 | g3646128 | thioredoxin-like protein |
| 1019 | rat00225625 | g3646128 | thioredoxin-like protein |
| 1020 | rat00052754 | g3646128 | thioredoxin-like protein |
| 1021 | rat00125754 | g3646128 | thioredoxin-like protein |
| 1022 | rat00216555 | g3646128 | thioredoxin-like protein |
| 1023 | rat00249461 | g3646128 | thioredoxin-like protein |
| 1024 | rat00085625 | g3646128 | thioredoxin-like protein |
| 1025 | rat00138547 | g3646128 | thioredoxin-like protein |
| 1026 | rat00166582 | g3646128 | thioredoxin-like protein |
| 1027 | rat00245747 | g3646128 | thioredoxin-like protein |
| 1028 | rat00177618 | g3646128 | thioredoxin-like protein |
| 1029 | rat00249944 | g3646128 | thioredoxin-like protein |
| 1030 | rat00000920 | g3646128 | thioredoxin-like protein |
| 1031 | rat00121523 | g3646128 | thioredoxin-like protein |
| 1032 | rat00181521 | g3646128 | thioredoxin-like protein |
| 1033 | rat00214904 | g3646128 | thioredoxin-like protein |
| 1034 | rat00052481 | g3646128 | thioredoxin-like protein |
| 1035 | rat00134751 | g3646128 | thioredoxin-like protein |
| 1036 | rat00022566 | g3646128 | thioredoxin-like protein |
| 1037 | rat00040368 | g3646128 | thioredoxin-like protein |
| 1038 | rat00008580 | g3646128 | thioredoxin-like protein |
| 1039 | rat00059250 | g3646128 | thioredoxin-like protein |
| 1040 | rat00191560 | g3646128 | thioredoxin-like protein |
| 1041 | rat00017815 | g3646128 | thioredoxin-like protein |
| 1042 | hu00042860 | g37118 | Human gene for thioredoxin, exons 2 and 3. |
| 1043 | hu00298010 | g37118 | Human gene for thioredoxin, exons 2 and 3. |
| 1044 | hu00933420 | g37118 | Human gene for thioredoxin, exons 2 and 3. |
| 1045 | hu00667284 | g37118 | Human gene for thioredoxin, exons 2 and 3. |
| 1046 | hu00372039 | g181261 | Human somatic cytochrome c (HC6) processed pseudogene, complete cds. |
| 1047 | rat00124233 | g2443331 | Nfrl |
| 1048 | rat00239898 | g2443331 | Nfrl |
| 1049 | hu00551266 | g897580 | Human iron-regulatory protein 2 (IRP2) mRNA, partial cds. |
| 1050 | hu00666846 | g897580 | Human iron-regulatory protein 2 (IRP2) mRNA, partial cds. |
| 1051 | hu00528877 | g897580 | Human iron-regulatory protein 2 (IRP2) mRNA, partial cds. |
| 1052 | hu00815978 | g897580 | Human iron-regulatory protein 2 (IRP2) mRNA, partial cds. |
| 1053 | hu00638589 | g897580 | Human iron-regulatory protein 2 (IRP2) mRNA, partial cds. |
| 1054 | hu00031025 | g897580 | Human iron-regulatory protein 2 (IRP2) mRNA, partial cds. |
| 1055 | hu00499778 | g897580 | Human iron-regulatory protein 2 (IRP2) mRNA, partial cds. |
| 1056 | hu00678989 | g897580 | Human iron-regulatory protein 2 (IRP2) mRNA, partial cds. |
| 1057 | hu00016195 | g897580 | Human iron-regulatory protein 2 (IRP2) mRNA, partial cds. |
| 1058 | hu00442156 | g897580 | Human iron-regulatory protein 2 (IRP2) mRNA, partial cds. |
| 1059 | hu00952341 | g897580 | Human iron-regulatory protein 2 (IRP2) mRNA, partial cds. |
| 1060 | hu00905337 | g897580 | Human iron-regulatory protein 2 (IRP2) mRNA, partial cds. |
| 1061 | hu00839062 | g897580 | Human iron-regulatory protein 2 (IRP2) mRNA, partial cds. |
| 1062 | hu00804097 | g897580 | Human iron-regulatory protein 2 (IRP2) mRNA, partial cds. |
| 1063 | hu00867242 | g897580 | Human iron-regulatory protein 2 (IRP2) mRNA, partial cds. |
| 1064 | hu00346764 | g897580 | Human iron-regulatory protein 2 (IRP2) mRNA, partial cds. |
| 1065 | hu00733855 | g897580 | Human iron-regulatory protein 2 (IRP2) mRNA, partial cds. |
| 1066 | hu00505777 | g897580 | Human iron-regulatory protein 2 (IRP2) mRNA, partial cds. |
| 1067 | hu00439947 | g897580 | Human iron-regulatory protein 2 (IRP2) mRNA, partial cds. |
| 1068 | hu00898254 | g897580 | Human iron-regulatory protein 2 (IRP2) mRNA, partial cds. |
| 1069 | hu00043131 | g897580 | Human iron-regulatory protein 2 (IRP2) mRNA, partial cds. |
| 1070 | hu00141506 | g897580 | Human iron-regulatory protein 2 (IRP2) mRNA, partial cds. |
| 1071 | hu00561303 | g897580 | Human iron-regulatory protein 2 (IRP2) mRNA, partial cds. |
| 1072 | hu00046521 | g897580 | Human iron-regulatory protein 2 (IRP2) mRNA, partial cds. |
| 1073 | hu00159103 | g897580 | Human iron-regulatory protein 2 (IRP2) mRNA, partial cds. |
| 1074 | hu00313147 | g897580 | Human iron-regulatory protein 2 (IRP2) mRNA, partial cds. |
| 1075 | hu00901261 | g897580 | Human iron-regulatory protein 2 (IRP2) mRNA, partial cds. |
| 1076 | hu00627230 | g897580 | Human iron-regulatory protein 2 (IRP2) mRNA, partial cds. |
| 1077 | hu00203835 | g897580 | Human iron-regulatory protein 2 (IRP2) mRNA, partial cds. |
| 1078 | hu00386214 | g897580 | Human iron-regulatory protein 2 (IRP2) mRNA, partial cds. |
| 1079 | hu00923443 | g897580 | Human iron-regulatory protein 2 (IRP2) mRNA, partial cds. |
| 1080 | hu00726614 | g897580 | Human iron-regulatory protein 2 (IRP2) mRNA, partial cds. |
| 1081 | hu00324675 | g897580 | Human iron-regulatory protein 2 (IRP2) mRNA, partial cds. |
| 1082 | hu00616149 | g897580 | Human iron-regulatory protein 2 (IRP2) mRNA, partial cds. |
| 1083 | hu00823419 | g897580 | Human iron-regulatory protein 2 (IRP2) mRNA, partial cds. |
| 1084 | hu00367308 | g897580 | Human iron-regulatory protein 2 (IRP2) mRNA, partial cds. |
| 1085 | hu00554095 | g897580 | Human iron-regulatory protein 2 (IRP2) mRNA, partial cds. |
| 1086 | hu00647155 | g897580 | Human iron-regulatory protein 2 (IRP2) mRNA, partial cds. |
| 1087 | hu00761366 | g897580 | Human iron-regulatory protein 2 (IRP2) mRNA, partial cds. |
| 1088 | hu00783265 | g897580 | Human iron-regulatory protein 2 (IRP2) mRNA, partial cds. |
| 1089 | hu00540697 | g897580 | Human iron-regulatory protein 2 (IRP2) mRNA, partial cds. |
| 1090 | hu00881555 | g897580 | Human iron-regulatory protein 2 (IRP2) mRNA, partial cds. |
| 1091 | hu00457495 | g897580 | Human iron-regulatory protein 2 (IRP2) mRNA, partial cds. |
| 1092 | hu00364798 | g897580 | Human iron-regulatory protein 2 (IRP2) mRNA, partial cds. |
| 1093 | hu00110621 | g897580 | Human iron-regulatory protein 2 (IRP2) mRNA, partial cds. |
| 1094 | hu00277043 | g897580 | Human iron-regulatory protein 2 (IRP2) mRNA, partial cds. |
| 1095 | hu00201587 | g897580 | Human iron-regulatory protein 2 (IRP2) mRNA, partial cds. |
| 1096 | hu00320966 | g897580 | Human iron-regulatory protein 2 (IRP2) mRNA, partial cds. |
| 1097 | hu00693044 | g897580 | Human iron-regulatory protein 2 (IRP2) mRNA, partial cds. |
| 1098 | hu00634454 | g897580 | Human iron-regulatory protein 2 (IRP2) mRNA, partial cds. |
| 1099 | hu00711495 | g897580 | Human iron-regulatory protein 2 (IRP2) mRNA, partial cds. |
| 1100 | hu00610579 | g897580 | Human iron-regulatory protein 2 (IRP2) mRNA, partial cds. |
| 1101 | hu00340966 | g897580 | Human iron-regulatory protein 2 (IRP2) mRNA, partial cds. |
| 1102 | hu00447662 | g897580 | Human iron-regulatory protein 2 (IRP2) mRNA, partial cds. |
| 1103 | hu00267528 | g897580 | Human iron-regulatory protein 2 (IRP2) mRNA, partial cds. |
| 1104 | hu00570983 | g897580 | Human iron-regulatory protein 2 (IRP2) mRNA, partial cds. |
| 1105 | hu00589236 | g897580 | Human iron-regulatory protein 2 (IRP2) mRNA, partial cds. |
| 1106 | hu00494959 | g897580 | Human iron-regulatory protein 2 (IRP2) mRNA, partial cds. |
| 1107 | hu00854000 | g897580 | Human iron-regulatory protein 2 (IRP2) mRNA, partial cds. |
| 1108 | hu00155044 | g897580 | Human iron-regulatory protein 2 (IRP2) mRNA, partial cds. |
| 1109 | hu00622162 | g897580 | Human iron-regulatory protein 2 (IRP2) mRNA, partial cds. |
| 1110 | hu00304814 | g897580 | Human iron-regulatory protein 2 (IRP2) mRNA, partial cds. |
| 1111 | hu00008564 | g897580 | Human iron-regulatory protein 2 (IRP2) mRNA, partial cds. |
| 1112 | hu00191328 | g897580 | Human iron-regulatory protein 2 (IRP2) mRNA, partial cds. |
| 1113 | hu00652363 | g897580 | Human iron-regulatory protein 2 (IRP2) mRNA, partial cds. |
| 1114 | hu00760337 | g897580 | Human iron-regulatory protein 2 (IRP2) mRNA, partial cds. |
| 1115 | hu00482708 | g897580 | Human iron-regulatory protein 2 (IRP2) mRNA, partial cds. |
| 1116 | hu00342677 | g897580 | Human iron-regulatory protein 2 (IRP2) mRNA, partial cds. |
| 1117 | hu00495256 | g897580 | Human iron-regulatory protein 2 (IRP2) mRNA, partial cds. |
| 1118 | hu00264785 | g897580 | Human iron-regulatory protein 2 (IRP2) mRNA, partial cds. |
| 1119 | hu00334698 | g897580 | Human iron-regulatory protein 2 (IRP2) mRNA, partial cds. |
| 1120 | hu00939481 | g897580 | Human iron-regulatory protein 2 (IRP2) mRNA, partial cds. |
| 1121 | rat00219199 | g3603233 | Mouse type II peroxiredoxin 1 (PrxII-1) gene, promoter region and exon 1. |
| 1122 | rat00177802 | g3603233 | Mouse type II peroxiredoxin 1 (PrxII-1) gene, promoter region and exon 1. |
| 1123 | rat00179794 | g3603233 | Mouse type II peroxiredoxin 1 (PrxII-1) gene, promoter region and exon 1. |
| 1124 | rat00199742 | g3603233 | Mouse type II peroxiredoxin 1 (PrxII-1) gene, promoter region and exon 1. |
| 1125 | hu00370477 | g181234 | Human p22-phox (CYBA) gene, exon 5. |
| 1126 | hu00201755 | g181234 | Human p22-phox (CYBA) gene, exon 5. |
| 1127 | hu00352946 | g181234 | Human p22-phox (CYBA) gene, exon 5. |
| 1128 | hu00422951 | g181234 | Human p22-phox (CYBA) gene, exon 5. |
| 1129 | hu00509064 | g181234 | Human p22-phox (CYBA) gene, exon 5. |
| 1130 | hu01263994 | g3171877 | dJ127D3.2 (Flavin-containing Monooxygenase family protein) |
| 1131 | hu01115208 | g453964 | ATL-derived factor/thioredoxin |
| 1132 | hu01072755 | g453963 | Human mRNA for ATL-derived factor/thiredoxin. |
| 1133 | hu01005858 | g453963 | Human mRNA for ATL-derived factor/thiredoxin. |
| 1134 | hu01065170 | g453963 | Human mRNA for ATL-derived factor/thiredoxin. |
| 1135 | hu01198782 | g453963 | Human mRNA for ATL-derived factor/thiredoxin. |
| 1136 | hu01148976 | g453963 | Human mRNA for ATL-derived factor/thiredoxin. |
| 1137 | hu01163198 | g453963 | Human mRNA for ATL-derived factor/thiredoxin. |
| 1138 | hu01281639 | g453963 | Human mRNA for ATL-derived factor/thiredoxin. |
| 1139 | hu01123628 | g453963 | Human mRNA for ATL-derived factor/thiredoxin. |
| 1140 | hu01154039 | g453963 | Human mRNA for ATL-derived factor/thiredoxin. |
| 1141 | hu01206351 | g453963 | Human mRNA for ATL-derived factor/thiredoxin. |
| 1142 | hu01014732 | g453963 | Human mRNA for ATL-derived factor/thiredoxin. |
| 1143 | hu00986147 | g453963 | Human mRNA for ATL-derived factor/thiredoxin. |
| 1144 | hu01215972 | g453963 | Human mRNA for ATL-derived factor/thiredoxin. |
| 1145 | hu01051075 | g453963 | Human mRNA for ATL-derived factor/thiredoxin. |
| 1146 | hu01218852 | g453963 | Human mRNA for ATL-derived factor/thiredoxin. |
| 1147 | hu01158132 | g453963 | Human mRNA for ATL-derived factor/thiredoxin. |
| 1148 | hu01011622 | g453963 | Human mRNA for ATL-derived factor/thiredoxin. |
| 1149 | hu01339166 | g453963 | Human mRNA for ATL-derived factor/thiredoxin. |
| 1150 | hu01034696 | g453963 | Human mRNA for ATL-derived factor/thiredoxin. |
| 1151 | hu01159033 | g453963 | Human mRNA for ATL-derived factor/thiredoxin. |
| 1152 | hu00994745 | g453963 | Human mRNA for ATL-derived factor/thiredoxin. |
| 1153 | hu01054653 | g453963 | Human mRNA for ATL-derived factor/thiredoxin. |
| 1154 | hu01307184 | g453963 | Human mRNA for ATL-derived factor/thiredoxin. |
| 1155 | hu01195431 | g453963 | Human mRNA for ATL-derived factor/thiredoxin. |
| 1156 | hu01302613 | g453963 | Human mRNA for ATL-derived factor/thiredoxin. |
| 1157 | hu01191060 | g453963 | Human mRNA for ATL-derived factor/thiredoxin. |
| 1158 | rat00177217 | g677891 | cytochrome b558 alpha-subunit |
| 1159 | rat00049893 | g677891 | cytochrome b558 alpha-subunit |
| 1160 | rat00187352 | g677891 | cytochrome b558 alpha-subunit |
| 1161 | rat00114238 | g677891 | cytochrome b558 alpha-subunit |
| 1162 | rat00177786 | g677891 | cytochrome b558 alpha-subunit |
| 1163 | rat00136648 | g677891 | cytochrome b558 alpha-subunit |
| 1164 | rat00046766 | g677891 | cytochrome b558 alpha-subunit |
| 1165 | rat00200496 | g677891 | cytochrome b558 alpha-subunit |
| 1166 | rat00100831 | g677891 | cytochrome b558 alpha-subunit |
| 1167 | rat00132474 | g677891 | cytochrome b558 alpha-subunit |
| 1168 | rat00144368 | g3881842 | Similarity to E. coli 2-oxoglutarate dehydrogenase (SW:ODO1_ECOLI); cDNA EST |
| 1169 | rat00168417 | g3881842 | Similarity to E. coli 2-oxoglutarate dehydrogenase (SW:ODO1_ECOLI); cDNA EST |
| 1170 | rat00076851 | g3881842 | Similarity to E. coli 2-oxoglutarate dehydrogenase (SW:ODO1_ECOLI); cDNA EST |
| 1171 | rat00216982 | g3881842 | Similarity to E. coli 2-oxoglutarate dehydrogenase (SW:ODO1_ECOLI); cDNA EST |
| 1172 | rat00248339 | g3881842 | Similarity to E. coli 2-oxoglutarate dehydrogenase (SW:ODO1_ECOLI); cDNA EST |
| 1173 | rat00213314 | g3881842 | Similarity to E. coli 2-oxoglutarate dehydrogenase (SW:ODO1_ECOLI); cDNA EST |
| 1174 | rat00105136 | g3881842 | Similarity to E. coli 2-oxoglutarate dehydrogenase (SW:ODO1_ECOLI); cDNA EST |
| 1175 | rat00130615 | g3881842 | Similarity to E. coli 2-oxoglutarate dehydrogenase (SW:ODO1_ECOLI); cDNA EST |
| 1176 | rat00251888 | g3881842 | Similarity to E. coli 2-oxoglutarate dehydrogenase (SW:ODO1_ECOLI); cDNA EST |
| 1177 | rat00221660 | g3881842 | Similarity to E. coli 2-oxoglutarate dehydrogenase (SW:ODO1_ECOLI); cDNA EST |
| 1178 | rat00042254 | g3881842 | Similarity to E. coli 2-oxoglutarate dehydrogenase (SW:ODO1_ECOLI); cDNA EST |
| 1179 | rat00010468 | g3881842 | Similarity to E. coli 2-oxoglutarate dehydrogenase (SW:ODO1_ECOLI); cDNA EST |
| 1180 | rat00120763 | g3881842 | Similarity to E. coli 2-oxoglutarate dehydrogenase (SW:ODO1_ECOLI); cDNA EST |
| 1181 | rat00028407 | g3881842 | Similarity to E. coli 2-oxoglutarate dehydrogenase (SW:ODO1_ECOLI); cDNA EST |
| 1182 | rat00107373 | g3881842 | Similarity to E. coli 2-oxoglutarate dehydrogenase (SW:ODO1_ECOLI); cDNA EST |
| 1183 | rat00121848 | g3881842 | Similarity to E. coli 2-oxoglutarate dehydrogenase (SW:ODO1_ECOLI); cDNA EST |
| 1184 | hu00475533 | g181266 | Human somatic cytochrome c (HS7) processed pseudogene, complete cds. |
| 1185 | hu00764500 | g181266 | Human somatic cytochrome c (HS7) processed pseudogene, complete cds. |
| 1186 | hu00589051 | g984591 | Human gene for 2-oxoglutarate dehydrogenase, exon 1 sequence. |
| 1187 | hu00518649 | g984591 | Human gene for 2-oxoglutarate dehydrogenase, exon 1 sequence. |
| 1188 | hu00267484 | g984611 | Human gene for 2-oxoglutarate dehydrogenase, exon 14, 15, 16, 17, 18 and 19 sequence. |
| 1189 | hu00467745 | g984611 | Human gene for 2-oxoglutarate dehydrogenase, exon 14, 15, 16, 17, 18 and 19 sequence. |
| 1190 | hu00866865 | g2897941 | Human thioredoxin-like protein mRNA, complete cds. |
| 1191 | hu00679638 | g2897941 | Human thioredoxin-like protein mRNA, complete cds. |
| 1192 | hu00000052 | g2897941 | Human thioredoxin-like protein mRNA, complete cds. |
| 1193 | hu00679918 | g2897941 | Human thioredoxin-like protein mRNA, complete cds. |
| 1194 | hu00221048 | g2897941 | Human thioredoxin-like protein mRNA, complete cds. |
| 1195 | hu00333450 | g2897941 | Human thioredoxin-like protein mRNA, complete cds. |
| 1196 | hu00236251 | g2897941 | Human thioredoxin-like protein mRNA, complete cds. |
| 1197 | hu00013962 | g2897941 | Human thioredoxin-like protein mRNA, complete cds. |
| 1198 | hu00077601 | g2897941 | Human thioredoxin-like protein mRNA, complete cds. |
| 1199 | hu00342680 | g2897941 | Human thioredoxin-like protein mRNA, complete cds. |
| 1200 | hu00475701 | g2897941 | Human thioredoxin-like protein mRNA, complete cds. |
| 1201 | hu00295127 | g2897941 | Human thioredoxin-like protein mRNA, complete cds. |
| 1202 | hu00005869 | g2897941 | Human thioredoxin-like protein mRNA, complete cds. |
| 1203 | hu00918443 | g2897941 | Human thioredoxin-like protein mRNA, complete cds. |
| 1204 | hu00750908 | g2897941 | Human thioredoxin-like protein mRNA, complete cds. |
| 1205 | hu00735159 | g2897941 | Human thioredoxin-like protein mRNA, complete cds. |
| 1206 | hu00349275 | g2897941 | Human thioredoxin-like protein mRNA, complete cds. |
| 1207 | hu00757851 | g2897941 | Human thioredoxin-like protein mRNA, complete cds. |
| 1208 | hu00645964 | g2897941 | Human thioredoxin-like protein mRNA, complete cds. |
| 1209 | hu00599372 | g2897941 | Human thioredoxin-like protein mRNA, complete cds. |
| 1210 | hu00466677 | g2897941 | Human thioredoxin-like protein mRNA, complete cds. |
| 1211 | hu00871867 | g2897941 | Human thioredoxin-like protein mRNA, complete cds. |
| 1212 | hu00669884 | g2897941 | Human thioredoxin-like protein mRNA, complete cds. |
| 1213 | hu00032005 | g2897941 | Human thioredoxin-like protein mRNA, complete cds. |
| 1214 | hu00406042 | g2897941 | Human thioredoxin-like protein mRNA, complete cds. |
| 1215 | hu00023490 | g2897941 | Human thioredoxin-like protein mRNA, complete cds. |
| 1216 | hu00537201 | g2897941 | Human thioredoxin-like protein mRNA, complete cds. |
| 1217 | hu00413338 | g2897941 | Human thioredoxin-like protein mRNA, complete cds. |
| 1218 | hu00086921 | g2897941 | Human thioredoxin-like protein mRNA, complete cds. |
| 1219 | hu00822446 | g2897941 | Human thioredoxin-like protein mRNA, complete cds. |
| 1220 | hu00369125 | g2897941 | Human thioredoxin-like protein mRNA, complete cds. |
| 1221 | hu00149385 | g2897941 | Human thioredoxin-like protein mRNA, complete cds. |
| 1222 | hu00088909 | g2897941 | Human thioredoxin-like protein mRNA, complete cds. |
| 1223 | hu00289537 | g2897941 | Human thioredoxin-like protein mRNA, complete cds. |
| 1224 | hu00465993 | g2897941 | Human thioredoxin-like protein mRNA, complete cds. |
| 1225 | hu00709688 | g2897941 | Human thioredoxin-like protein mRNA, complete cds. |
| 1226 | hu00785849 | g2897941 | Human thioredoxin-like protein mRNA, complete cds. |
| 1227 | hu00071401 | g2897941 | Human thioredoxin-like protein mRNA, complete cds. |
| 1228 | hu00133016 | g2897941 | Human thioredoxin-like protein mRNA, complete cds. |
| 1229 | hu00712341 | g2897941 | Human thioredoxin-like protein mRNA, complete cds. |
| 1230 | hu00651621 | g2897941 | Human thioredoxin-like protein mRNA, complete cds. |
| 1231 | hu00801723 | g2897941 | Human thioredoxin-like protein mRNA, complete cds. |
| 1232 | hu00325174 | g2897941 | Human thioredoxin-like protein mRNA, complete cds. |
| 1233 | hu00123496 | g2897941 | Human thioredoxin-like protein mRNA, complete cds. |
| 1234 | hu00644372 | g2897941 | Human thioredoxin-like protein mRNA, complete cds. |
| 1235 | hu00452495 | g2897941 | Human thioredoxin-like protein mRNA, complete cds. |
| 1236 | hu00667723 | g2897941 | Human thioredoxin-like protein mRNA, complete cds. |
| 1237 | hu00774680 | g2897941 | Human thioredoxin-like protein mRNA, complete cds. |
| 1238 | hu00908188 | g2897941 | Human thioredoxin-like protein mRNA, complete cds. |
| 1239 | hu00896372 | g2897941 | Human thioredoxin-like protein mRNA, complete cds. |
| 1240 | hu00095768 | g2897941 | Human thioredoxin-like protein mRNA, complete cds. |
| 1241 | hu00083110 | g2897941 | Human thioredoxin-like protein mRNA, complete cds. |
| 1242 | hu00241234 | g2897941 | Human thioredoxin-like protein mRNA, complete cds. |
| 1243 | hu00305063 | g2897941 | Human thioredoxin-like protein mRNA, complete cds. |
| 1244 | hu00377000 | g2897941 | Human thioredoxin-like protein mRNA, complete cds. |
| 1245 | hu00282403 | g2897941 | Human thioredoxin-like protein mRNA, complete cds. |
| 1246 | hu00003638 | g2897941 | Human thioredoxin-like protein mRNA, complete cds. |
| 1247 | hu00524118 | g2897941 | Human thioredoxin-like protein mRNA, complete cds. |
| 1248 | hu00307641 | g2897941 | Human thioredoxin-like protein mRNA, complete cds. |
| 1249 | hu00040648 | g2897941 | Human thioredoxin-like protein mRNA, complete cds. |
| 1250 | hu00116735 | g2897941 | Human thioredoxin-like protein mRNA, complete cds. |
| 1251 | hu00344058 | g2897941 | Human thioredoxin-like protein mRNA, complete cds. |
| 1252 | hu00060966 | g2897941 | Human thioredoxin-like protein mRNA, complete cds. |
| 1253 | hu00458246 | g2897941 | Human thioredoxin-like protein mRNA, complete cds. |
| 1254 | hu00298623 | g2897941 | Human thioredoxin-like protein mRNA, complete cds. |
| 1255 | hu00052869 | g2897941 | Human thioredoxin-like protein mRNA, complete cds. |
| 1256 | hu00530798 | g2897941 | Human thioredoxin-like protein mRNA, complete cds. |
| 1257 | hu00961646 | g2897941 | Human thioredoxin-like protein mRNA, complete cds. |
| 1258 | hu00948713 | g2897941 | Human thioredoxin-like protein mRNA, complete cds. |
| 1259 | hu00547548 | g2897941 | Human thioredoxin-like protein mRNA, complete cds. |
| 1260 | rat00151369 | g2351037 | cytochrome b small subunit of complex II |
| 1261 | rat00127186 | g2351037 | cytochrome b small subunit of complex II |
| 1262 | rat00069932 | g2351037 | cytochrome b small subunit of complex II |
| 1263 | rat00192972 | g2351037 | cytochrome b small subunit of complex II |
| 1264 | rat00035697 | g2351037 | cytochrome b small subunit of complex II |
| 1265 | rat00026987 | g2351037 | cytochrome b small subunit of complex II |
| 1266 | rat00163788 | g2351037 | cytochrome b small subunit of complex II |
| 1267 | rat00168756 | g2351037 | cytochrome b small subunit of complex II |
| 1268 | rat00000372 | g2351037 | cytochrome b small subunit of complex II |
| 1269 | rat00202332 | g2351037 | cytochrome b small subunit of complex II |
| 1270 | rat00110821 | g2351037 | cytochrome b small subunit of complex II |
| 1271 | rat00010175 | g2351037 | cytochrome b small subunit of complex II |
| 1272 | rat00190601 | g2351037 | cytochrome b small subunit of complex II |
| 1273 | rat00085202 | g2351037 | cytochrome b small subunit of complex II |
| 1274 | rat00165071 | g2351037 | cytochrome b small subunit of complex II |
| 1275 | rat00035607 | g2351037 | cytochrome b small subunit of complex II |
| 1276 | rat00162742 | g2351037 | cytochrome b small subunit of complex II |
| 1277 | rat00080178 | g2351037 | cytochrome b small subunit of complex II |
| 1278 | rat00206754 | g2351037 | cytochrome b small subunit of complex II |
| 1279 | rat00244717 | g2351037 | cytochrome b small subunit of complex II |
| 1280 | rat00078375 | g2351037 | cytochrome b small subunit of complex II |
| 1281 | rat00218796 | g2351037 | cytochrome b small subunit of complex II |
| 1282 | rat00112251 | g2351037 | cytochrome b small subunit of complex II |
| 1283 | rat00204007 | g2351037 | cytochrome b small subunit of complex II |
| 1284 | rat00171140 | g2351037 | cytochrome b small subunit of complex II |
| 1285 | rat00131225 | g2351037 | cytochrome b small subunit of complex II |
| 1286 | rat00125440 | g2351037 | cytochrome b small subunit of complex II |
| 1287 | rat00170710 | g2351037 | cytochrome b small subunit of complex II |
| 1288 | rat00125900 | g2351037 | cytochrome b small subunit of complex II |
| 1289 | rat00207810 | g2351037 | cytochrome b small subunit of complex II |
| 1290 | rat00114355 | g2351037 | cytochrome b small subunit of complex II |
| 1291 | rat00252245 | g2351037 | cytochrome b small subunit of complex II |
| 1292 | rat00059151 | g2351037 | cytochrome b small subunit of complex II |
| 1293 | rat00095233 | g2351037 | cytochrome b small subunit of complex II |
| 1294 | rat00024880 | g2351037 | cytochrome b small subunit of complex II |
| 1295 | rat00036707 | g2351037 | cytochrome b small subunit of complex II |
| 1296 | rat00227417 | g2351037 | cytochrome b small subunit of complex II |
| 1297 | rat00150890 | g2351037 | cytochrome b small subunit of complex II |
| 1298 | rat00252960 | g2351037 | cytochrome b small subunit of complex II |
| 1299 | rat00038841 | g2351037 | cytochrome b small subunit of complex II |
| 1300 | hu00538491 | g984599 | Human gene for 2-oxoglutarate dehydrogenase, exon 5, 6, 7 and 8 sequence. |
| 1301 | hu00384197 | g339648 | Human thioredoxin (TXN) mRNA, complete cds. |
| 1302 | rat00200270 | g220729 | Rat mRNA for cytochrome b5. |
| 1303 | rat00094481 | g220729 | Rat mRNA for cytochrome b5. |
| 1304 | rat00210583 | g220729 | Rat mRNA for cytochrome b5. |
| 1305 | rat00181406 | g220729 | Rat mRNA for cytochrome b5. |
| 1306 | rat00028541 | g220729 | Rat mRNA for cytochrome b5. |
| 1307 | rat00216462 | g220729 | Rat mRNA for cytochrome b5. |
| 1308 | rat00133499 | g220729 | Rat mRNA for cytochrome b5. |
| 1309 | rat00254102 | g220729 | Rat mRNA for cytochrome b5. |
| 1310 | rat00205869 | g220729 | Rat mRNA for cytochrome b5. |
| 1311 | rat00151088 | g220729 | Rat mRNA for cytochrome b5. |
| 1312 | rat00170519 | g220729 | Rat mRNA for cytochrome b5. |
| 1313 | rat00083093 | g220729 | Rat mRNA for cytochrome b5. |
| 1314 | rat00246380 | g220729 | Rat mRNA for cytochrome b5. |
| 1315 | hu01278189 | g181391 | Human cytochrome b5 mRNA, complete cds. |
| 1316 | hu01055764 | g181391 | Human cytochrome b5 mRNA, complete cds. |
| 1317 | hu01211996 | g181391 | Human cytochrome b5 mRNA, complete cds. |
| 1318 | hu01043275 | g181391 | Human cytochrome b5 mRNA, complete cds. |
| 1319 | hu01015901 | g181391 | Human cytochrome b5 mRNA, complete cds. |
| 1320 | hu01344599 | g703083 | cytochrome b5 |
| 1321 | hu00996714 | g703083 | cytochrome b5 |
| 1322 | hu01111011 | g703083 | cytochrome b5 |
| 1323 | hu01349589 | g703083 | cytochrome b5 |
| 1324 | hu01033003 | g703083 | cytochrome b5 |
| 1325 | hu00998594 | g703083 | cytochrome b5 |
| 1326 | hu01103912 | g703083 | cytochrome b5 |
| 1327 | hu01261723 | g703083 | cytochrome b5 |
| 1328 | hu01263146 | g703083 | cytochrome b5 |
| 1329 | hu01278213 | g703083 | cytochrome b5 |
| 1330 | hu01152645 | g703083 | cytochrome b5 |
| 1331 | hu01156704 | g703083 | cytochrome b5 |
| 1332 | hu01245940 | g703083 | cytochrome b5 |
| 1333 | hu01082536 | g703083 | cytochrome b5 |
| 1334 | hu01204479 | g703083 | cytochrome b5 |
| 1335 | hu01266736 | g703083 | cytochrome b5 |
| 1336 | hu01165873 | g703083 | cytochrome b5 |
| 1337 | hu01028859 | g703083 | cytochrome b5 |
| 1338 | hu01168945 | g703083 | cytochrome b5 |
| 1339 | hu01159774 | g703083 | cytochrome b5 |
| 1340 | hu00979550 | g703083 | cytochrome b5 |
| 1341 | hu01130335 | g703083 | cytochrome b5 |
| 1342 | hu01302830 | g703083 | cytochrome b5 |
| 1343 | hu01103129 | g703083 | cytochrome b5 |
| 1344 | hu01019114 | g703083 | cytochrome b5 |
| 1345 | hu01311940 | g703083 | cytochrome b5 |
| 1346 | hu01009470 | g703083 | cytochrome b5 |
| 1347 | hu01171822 | g703083 | cytochrome b5 |
| 1348 | hu01285132 | g703083 | cytochrome b5 |
| 1349 | hu01215652 | g703083 | cytochrome b5 |
| 1350 | hu01293414 | g703083 | cytochrome b5 |
| 1351 | hu01120975 | g703083 | cytochrome b5 |
| 1352 | rat00036395 | g1217592 | Mouse mRNA for squalene epoxidase, complete cds. |
| 1353 | rat00028422 | g1217592 | Mouse mRNA for squalene epoxidase, complete cds. |
| 1354 | rat00118389 | g1217592 | Mouse mRNA for squalene epoxidase, complete cds. |
| 1355 | rat00216886 | g1217592 | Mouse mRNA for squalene epoxidase, complete cds. |
| 1356 | rat00099149 | g1217592 | Mouse mRNA for squalene epoxidase, complete cds. |
| 1357 | hu01012121 | g1651201 | cytochrome b561 |
| 1358 | hu01242117 | g1651201 | cytochrome b561 |
| 1359 | hu01183411 | g1651201 | cytochrome b561 |
| 1360 | hu01031536 | g1651201 | cytochrome b561 |
| 1361 | hu01213381 | g1651201 | cytochrome b561 |
| 1362 | hu01149122 | g1651201 | cytochrome b561 |
| 1363 | hu01124822 | g1651201 | cytochrome b561 |
| 1364 | hu01266024 | g1651201 | cytochrome b561 |
| 1365 | hu01124184 | g1651201 | cytochrome b561 |
| 1366 | hu01086601 | g1651201 | cytochrome b561 |
| 1367 | hu01070384 | g1651201 | cytochrome b561 |
| 1368 | hu01073382 | g1651201 | cytochrome b561 |
| 1369 | hu01031739 | g1651201 | cytochrome b561 |
| 1370 | hu01115358 | g1651201 | cytochrome b561 |
| 1371 | hu01153717 | g1651201 | cytochrome b561 |
| 1372 | hu01025981 | g1651201 | cytochrome b561 |
| 1373 | hu01303793 | g1651201 | cytochrome b561 |
| 1374 | hu01227177 | g1651201 | cytochrome b561 |
| 1375 | hu01239409 | g1651201 | cytochrome b561 |
| 1376 | hu01178354 | g1651201 | cytochrome b561 |
| 1377 | hu01269164 | g1651201 | cytochrome b561 |
| 1378 | hu01348841 | g1651201 | cytochrome b561 |
| 1379 | hu00531731 | g1651201 | cytochrome b561 |
| 1380 | hu00485095 | g1651201 | cytochrome b561 |
| 1381 | hu00443053 | g476590 | Human cytochrome B561, HCYTO B561, mRNA, partial cds. |
| 1382 | hu00688152 | g476590 | Human cytochrome B561, HCYTO B561, mRNA, partial cds. |
| 1383 | hu00592769 | g476590 | Human cytochrome B561, HCYTO B561, mRNA, partial cds. |
| 1384 | hu00498963 | g476590 | Human cytochrome B561, HCYTO B561, mRNA, partial cds. |
| 1385 | hu00213468 | g476590 | Human cytochrome B561, HCYTO B561, mRNA, partial cds. |
| 1386 | hu00686498 | g476590 | Human cytochrome B561, HCYTO B561, mRNA, partial cds. |
| 1387 | hu00154170 | g476590 | Human cytochrome B561, HCYTO B561, mRNA, partial cds. |
| 1388 | hu00747944 | g30302 | Human mRNA for cytochrome c1. |
| 1389 | hu00685584 | g30302 | Human mRNA for cytochrome c1. |
| 1390 | hu00547949 | g30302 | Human mRNA for cytochrome c1. |
| 1391 | hu00030222 | g30302 | Human mRNA for cytochrome c1. |
| 1392 | hu00720500 | g30302 | Human mRNA for cytochrome c1. |
| 1393 | hu00204075 | g30302 | Human mRNA for cytochrome c1. |
| 1394 | hu00035425 | g30302 | Human mRNA for cytochrome c1. |
| 1395 | hu00650413 | g30302 | Human mRNA for cytochrome c1. |
| 1396 | hu00315083 | g30302 | Human mRNA for cytochrome c1. |
| 1397 | hu00221835 | g30302 | Human mRNA for cytochrome c1. |
| 1398 | hu00497011 | g30302 | Human mRNA for cytochrome c1. |
| 1399 | hu00750217 | g30302 | Human mRNA for cytochrome c1. |
| 1400 | hu00397213 | g30302 | Human mRNA for cytochrome c1. |
| 1401 | hu00256912 | g30302 | Human mRNA for cytochrome c1. |
| 1402 | hu00181475 | g30302 | Human mRNA for cytochrome c1. |
| 1403 | hu00449580 | g30302 | Human mRNA for cytochrome c1. |
| 1404 | hu00716908 | g30302 | Human mRNA for cytochrome c1. |
| 1405 | hu00773584 | g30302 | Human mRNA for cytochrome c1. |
| 1406 | hu00040407 | g1651199 | cytochrome b561 |
| 1407 | hu01076192 | g939707 | cytochrome b561 |
| 1408 | hu00923221 | g1651201 | cytochrome b561 |
| 1409 | hu00007264 | g1651201 | cytochrome b561 |
| 1410 | hu00864391 | g1651201 | cytochrome b561 |
| 1411 | hu00384233 | g1651201 | cytochrome b561 |
| 1412 | hu00553586 | g1651201 | cytochrome b561 |
| 1413 | hu00385647 | g1651201 | cytochrome b561 |
| 1414 | hu00466019 | g1651201 | cytochrome b561 |
| 1415 | hu00051632 | g181391 | Human cytochrome b5 mRNA, complete cds. |
| 1416 | hu00950968 | g181226 | Human cytochrome b5 mRNA, complete cds. |
| 1417 | hu00155335 | g181226 | Human cytochrome b5 mRNA, complete cds. |
| 1418 | hu01034310 | g1651199 | cytochrome b561 |
| 1419 | hu01304800 | g1651199 | cytochrome b561 |
| 1420 | hu01260071 | g1651199 | cytochrome b561 |
| 1421 | hu01108602 | g1651199 | cytochrome b561 |
| 1422 | hu01085123 | g1651199 | cytochrome b561 |
| 1423 | hu01224452 | g1651199 | cytochrome b561 |
| 1424 | hu01116462 | g1651199 | cytochrome b561 |
| 1425 | hu01332724 | g1651199 | cytochrome b561 |
| 1426 | hu01205581 | g1651199 | cytochrome b561 |
| 1427 | hu01182443 | g1651199 | cytochrome b561 |
| 1428 | hu01144509 | g1651199 | cytochrome b561 |
| 1429 | hu01205385 | g1651199 | cytochrome b561 |
| 1430 | hu01320884 | g1651199 | cytochrome b561 |
| 1431 | hu01052792 | g1651199 | cytochrome b561 |
| 1432 | hu01312667 | g1651199 | cytochrome b561 |
| 1433 | hu01132488 | g1651199 | cytochrome b561 |
| 1434 | hu00972009 | g1651199 | cytochrome b561 |
| 1435 | hu01094916 | g1651199 | cytochrome b561 |
| 1436 | hu01158348 | g1651199 | cytochrome b561 |
| 1437 | hu01085909 | g1651199 | cytochrome b561 |
| 1438 | hu00987153 | g1651199 | cytochrome b561 |
| 1439 | hu00973753 | g1651199 | cytochrome b561 |
| 1440 | hu01328985 | g1651199 | cytochrome b561 |
| 1441 | hu01025091 | g1651199 | cytochrome b561 |
| 1442 | hu01042508 | g1651199 | cytochrome b561 |
| 1443 | hu01050366 | g1651199 | cytochrome b561 |
| 1444 | hu01316712 | g1651199 | cytochrome b561 |
| 1445 | hu01105484 | g1651199 | cytochrome b561 |
| 1446 | hu01197221 | g1651199 | cytochrome b561 |
| 1447 | hu01014269 | g1651199 | cytochrome b561 |
| 1448 | hu01119624 | g1651199 | cytochrome b561 |
| 1449 | hu01166370 | g1651199 | cytochrome b561 |
| 1450 | hu01290607 | g1651199 | cytochrome b561 |
| 1451 | hu01235356 | g1651199 | cytochrome b561 |
| 1452 | hu01213739 | g1651199 | cytochrome b561 |
| 1453 | hu01012967 | g1651199 | cytochrome b561 |
| 1454 | hu01347308 | g1651199 | cytochrome b561 |
| 1455 | hu01242764 | g1651199 | cytochrome b561 |
| 1456 | hu01141983 | g1651199 | cytochrome b561 |
| 1457 | hu00981027 | g1651199 | cytochrome b561 |
| 1458 | hu01225836 | g1651199 | cytochrome b561 |
| 1459 | hu01021995 | g1651199 | cytochrome b561 |
| 1460 | hu01072945 | g1651199 | cytochrome b561 |
| 1461 | hu01335646 | g1651199 | cytochrome b561 |
| 1462 | hu01252649 | g1651199 | cytochrome b561 |
| 1463 | hu00971282 | g1651199 | cytochrome b561 |
| 1464 | hu01294428 | g1651199 | cytochrome b561 |
| 1465 | hu00991381 | g1651199 | cytochrome b561 |
| 1466 | hu01304779 | g1651199 | cytochrome b561 |
| 1467 | hu01179067 | g1651199 | cytochrome b561 |
| 1468 | hu00992273 | g1651199 | cytochrome b561 |
| 1469 | hu01094568 | g1651199 | cytochrome b561 |
| 1470 | hu01286548 | g1651199 | cytochrome b561 |
| 1471 | hu01189762 | g1651199 | cytochrome b561 |
| 1472 | hu00378272 | g1651201 | cytochrome b561 |
| 1473 | hu00834376 | g1651201 | cytochrome b561 |
| 1474 | hu01095213 | g939707 | cytochrome b561 |
| 1475 | hu01109550 | g939707 | cytochrome b561 |
| 1476 | hu00979473 | g939707 | cytochrome b561 |
| 1477 | hu00988392 | g939707 | cytochrome b561 |
| 1478 | hu01006476 | g939707 | cytochrome b561 |
| 1479 | hu01224429 | g939707 | cytochrome b561 |
| 1480 | hu01222954 | g939707 | cytochrome b561 |
| 1481 | hu01199977 | g939707 | cytochrome b561 |
| 1482 | hu01064917 | g939707 | cytochrome b561 |
| 1483 | hu01253388 | g939707 | cytochrome b561 |
| 1484 | hu01212406 | g939707 | cytochrome b561 |
| 1485 | hu01298669 | g476591 | HCYTO B561 |
| 1486 | hu00276005 | g181391 | Human cytochrome b5 mRNA, complete cds. |
| 1487 | hu00469919 | g181391 | Human cytochrome b5 mRNA, complete cds. |
| 1488 | hu00546016 | g181391 | Human cytochrome b5 mRNA, complete cds. |
| 1489 | hu00372163 | g181391 | Human cytochrome b5 mRNA, complete cds. |
| 1490 | hu00643640 | g181391 | Human cytochrome b5 mRNA, complete cds. |
| 1491 | hu00648878 | g181391 | Human cytochrome b5 mRNA, complete cds. |
| 1492 | hu00229760 | g181391 | Human cytochrome b5 mRNA, complete cds. |
| 1493 | hu00285912 | g181391 | Human cytochrome b5 mRNA, complete cds. |
| 1494 | hu00140054 | g181391 | Human cytochrome b5 mRNA, complete cds. |
| 1495 | hu00048012 | g181391 | Human cytochrome b5 mRNA, complete cds. |
| 1496 | hu00954177 | g181391 | Human cytochrome b5 mRNA, complete cds. |
| 1497 | hu00062127 | g181391 | Human cytochrome b5 mRNA, complete cds. |
| 1498 | hu00046072 | g1651201 | cytochrome b561 |
| 1499 | hu00205473 | g1651201 | cytochrome b561 |
| 1500 | hu00569891 | g1651201 | cytochrome b561 |
| 1501 | hu00779638 | g1651201 | cytochrome b561 |
| 1502 | hu00006783 | g1651201 | cytochrome b561 |
| 1503 | hu00393703 | g1651201 | cytochrome b561 |
| 1504 | hu00302668 | g1651201 | cytochrome b561 |
| 1505 | hu00560726 | g1651201 | cytochrome b561 |
| 1506 | hu00012332 | g1651201 | cytochrome b561 |
| 1507 | hu00732727 | g1651201 | cytochrome b561 |
| 1508 | hu00336872 | g1651201 | cytochrome b561 |
| 1509 | hu00851294 | g1651201 | cytochrome b561 |
| 1510 | hu00049358 | g1651201 | cytochrome b561 |
| 1511 | hu00489775 | g1651199 | cytochrome b561 |
| 1512 | hu00041104 | g1651199 | cytochrome b561 |
| 1513 | hu00141131 | g1651199 | cytochrome b561 |
| 1514 | hu00260911 | g1651199 | cytochrome b561 |
| 1515 | hu00518080 | g1651199 | cytochrome b561 |
| 1516 | hu00465900 | g1651199 | cytochrome b561 |
| 1517 | hu00381435 | g1651199 | cytochrome b561 |
| 1518 | hu00268512 | g1651199 | cytochrome b561 |
| 1519 | hu00639007 | g1651199 | cytochrome b561 |
| 1520 | hu00285128 | g1651199 | cytochrome b561 |
| 1521 | hu00485343 | g1651199 | cytochrome b561 |
| 1522 | hu00933750 | g1651199 | cytochrome b561 |
| 1523 | hu00915057 | g1651199 | cytochrome b561 |
| 1524 | hu00383850 | g1651199 | cytochrome b561 |
| 1525 | hu00634951 | g1651199 | cytochrome b561 |
| 1526 | hu00098416 | g1651199 | cytochrome b561 |
| 1527 | hu00458218 | g1651199 | cytochrome b561 |
| 1528 | hu00199732 | g1651199 | cytochrome b561 |
| 1529 | hu00583276 | g1651199 | cytochrome b561 |
| 1530 | hu00637386 | g1651199 | cytochrome b561 |
| 1531 | hu00398585 | g1651199 | cytochrome b561 |
| 1532 | hu00688557 | g1651199 | cytochrome b561 |
| 1533 | hu00131101 | g1651199 | cytochrome b561 |
| 1534 | hu00010027 | g1651199 | cytochrome b561 |
| 1535 | hu00500537 | g1651199 | cytochrome b561 |
| 1536 | hu00007175 | g1651199 | cytochrome b561 |
| 1537 | hu00452405 | g1651199 | cytochrome b561 |
| 1538 | hu00122835 | g1651199 | cytochrome b561 |
| 1539 | hu00143166 | g1651199 | cytochrome b561 |
| 1540 | hu00891231 | g1651199 | cytochrome b561 |
| 1541 | hu00742056 | g1651199 | cytochrome b561 |
| 1542 | hu00930375 | g1651199 | cytochrome b561 |
| 1543 | hu00955063 | g1651199 | cytochrome b561 |
| 1544 | hu00547562 | g1651199 | cytochrome b561 |
| 1545 | hu00541586 | g1651199 | cytochrome b561 |
| 1546 | hu00225299 | g1651199 | cytochrome b561 |
| 1547 | hu00870269 | g1651199 | cytochrome b561 |
| 1548 | hu00295892 | g1651199 | cytochrome b561 |
| 1549 | hu00619659 | g1651199 | cytochrome b561 |
| 1550 | hu00264334 | g1651199 | cytochrome b561 |
| 1551 | hu00612715 | g1651199 | cytochrome b561 |
| 1552 | hu00653360 | g1651199 | cytochrome b561 |
| 1553 | hu00275424 | g1651199 | cytochrome b561 |
| 1554 | hu00643409 | g1651199 | cytochrome b561 |
| 1555 | hu00594620 | g1651199 | cytochrome b561 |
| 1556 | hu00493542 | g1651199 | cytochrome b561 |
| 1557 | hu00529435 | g1651199 | cytochrome b561 |
| 1558 | hu00359417 | g1651199 | cytochrome b561 |
| 1559 | hu00547491 | g1651199 | cytochrome b561 |
| 1560 | hu00659429 | g1651199 | cytochrome b561 |
| 1561 | hu00891881 | g1651199 | cytochrome b561 |
| 1562 | hu00358686 | g1651199 | cytochrome b561 |
| 1563 | hu00568948 | g1651199 | cytochrome b561 |
| 1564 | hu00241110 | g1651199 | cytochrome b561 |
| 1565 | hu00143107 | g1651199 | cytochrome b561 |
| 1566 | hu00162134 | g1651199 | cytochrome b561 |
| 1567 | hu00473230 | g1651199 | cytochrome b561 |
| 1568 | hu00521605 | g1651199 | cytochrome b561 |
| 1569 | hu00249510 | g1651199 | cytochrome b561 |
| 1570 | hu00371740 | g1651199 | cytochrome b561 |
| 1571 | hu00343962 | g1651199 | cytochrome b561 |
| 1572 | hu00663094 | g1651199 | cytochrome b561 |
| 1573 | hu00945281 | g1651199 | cytochrome b561 |
| 1574 | hu00938315 | g1651199 | cytochrome b561 |
| 1575 | hu00790652 | g1651199 | cytochrome b561 |
| 1576 | hu00619751 | g1651199 | cytochrome b561 |
| 1577 | hu00925364 | g1651199 | cytochrome b561 |
| 1578 | hu00852465 | g1651199 | cytochrome b561 |
| 1579 | hu00179681 | g1651199 | cytochrome b561 |
| 1580 | hu00492188 | g1651199 | cytochrome b561 |
| 1581 | hu00097395 | g1651199 | cytochrome b561 |
| 1582 | hu00295593 | g1651199 | cytochrome b561 |
| 1583 | hu00834968 | g1651199 | cytochrome b561 |
| 1584 | hu00527111 | g1651199 | cytochrome b561 |
| 1585 | hu00047205 | g1651199 | cytochrome b561 |
| 1586 | hu00223860 | g1651199 | cytochrome b561 |
| 1587 | hu00571413 | g1651199 | cytochrome b561 |
| 1588 | hu00951133 | g1651199 | cytochrome b561 |
| 1589 | hu00047364 | g1651199 | cytochrome b561 |
| 1590 | hu00363337 | g1651199 | cytochrome b561 |
| 1591 | hu00235367 | g1651199 | cytochrome b561 |
| 1592 | hu00489105 | g1651199 | cytochrome b561 |
| 1593 | hu00424737 | g1651199 | cytochrome b561 |
| 1594 | hu00738027 | g1651199 | cytochrome b561 |
| 1595 | hu00765072 | g1651199 | cytochrome b561 |
| 1596 | hu00745318 | g1651199 | cytochrome b561 |
| 1597 | hu00526466 | g1651199 | cytochrome b561 |
| 1598 | hu00783137 | g1651199 | cytochrome b561 |
| 1599 | hu00860493 | g1651199 | cytochrome b561 |
| 1600 | hu00449185 | g1651199 | cytochrome b561 |
| 1601 | hu00879900 | g1651199 | cytochrome b561 |
| 1602 | hu00674677 | g1651199 | cytochrome b561 |
| 1603 | hu00668674 | g1651199 | cytochrome b561 |
| 1604 | hu00745879 | g1651199 | cytochrome b561 |
| 1605 | hu00030141 | g1651199 | cytochrome b561 |
| 1606 | hu00487889 | g1651199 | cytochrome b561 |
| 1607 | hu00707180 | g1651199 | cytochrome b561 |
| 1608 | hu00630168 | g1651199 | cytochrome b561 |
| 1609 | hu00081637 | g1651199 | cytochrome b561 |
| 1610 | hu00178019 | g1651199 | cytochrome b561 |
| 1611 | hu00157607 | g1651199 | cytochrome b561 |
| 1612 | hu00654282 | g1651199 | cytochrome b561 |
| 1613 | hu00087850 | g1651199 | cytochrome b561 |
| 1614 | hu00194690 | g1651199 | cytochrome b561 |
| 1615 | hu00841079 | g1651199 | cytochrome b561 |
| 1616 | hu00237395 | g1651199 | cytochrome b561 |
| 1617 | hu00582519 | g1651199 | cytochrome b561 |
| 1618 | hu00878143 | g1651199 | cytochrome b561 |
| 1619 | hu00857732 | g1651199 | cytochrome b561 |
| 1620 | hu00295854 | g1651199 | cytochrome b561 |
| 1621 | hu00486285 | g1651199 | cytochrome b561 |
| 1622 | hu00547289 | g1651199 | cytochrome b561 |
| 1623 | hu00557569 | g1651199 | cytochrome b561 |
| 1624 | hu00033303 | g1651199 | cytochrome b561 |
| 1625 | hu00131317 | g1651199 | cytochrome b561 |
| 1626 | hu00133091 | g1651199 | cytochrome b561 |
| 1627 | hu00285103 | g1651199 | cytochrome b561 |
| 1628 | hu00262270 | g1651199 | cytochrome b561 |
| 1629 | hu00390009 | g1651199 | cytochrome b561 |
| 1630 | hu00463062 | g1651199 | cytochrome b561 |
| 1631 | hu00065049 | g1651199 | cytochrome b561 |
| 1632 | hu00657523 | g1651199 | cytochrome b561 |
| 1633 | hu00641665 | g1651199 | cytochrome b561 |
| 1634 | hu00685460 | g1651199 | cytochrome b561 |
| 1635 | hu00701858 | g1651199 | cytochrome b561 |
| 1636 | hu00492296 | g1651199 | cytochrome b561 |
| 1637 | hu00914947 | g1651199 | cytochrome b561 |
| 1638 | hu00580985 | g1651199 | cytochrome b561 |
| 1639 | hu00869328 | g1651199 | cytochrome b561 |
| 1640 | hu00306113 | g1651199 | cytochrome b561 |
| 1641 | hu00041492 | g1651199 | cytochrome b561 |
| 1642 | hu00906055 | g1651199 | cytochrome b561 |
| 1643 | hu00031696 | g1651199 | cytochrome b561 |
| 1644 | hu00801890 | g1651199 | cytochrome b561 |
| 1645 | hu00171728 | g1651199 | cytochrome b561 |
| 1646 | hu00166931 | g1651199 | cytochrome b561 |
| 1647 | hu00745263 | g1651199 | cytochrome b561 |
| 1648 | hu00551367 | g1651199 | cytochrome b561 |
| 1649 | hu00867070 | g1651199 | cytochrome b561 |
| 1650 | hu00682084 | g1651199 | cytochrome b561 |
| 1651 | hu00363449 | g1651199 | cytochrome b561 |
| 1652 | hu00964039 | g1651199 | cytochrome b561 |
| 1653 | rat00069019 | g168747 | phytoene dehydrogenase |
| 1654 | rat00253574 | g168747 | phytoene dehydrogenase |
| 1655 | rat00054846 | g168747 | phytoene dehydrogenase |
| 1656 | rat00007795 | g168747 | phytoene dehydrogenase |
| 1657 | rat00176617 | g168747 | phytoene dehydrogenase |
| 1658 | rat00114994 | g168747 | phytoene dehydrogenase |
| 1659 | rat00209478 | g168747 | phytoene dehydrogenase |
| 1660 | rat00139486 | g168747 | phytoene dehydrogenase |
| 1661 | rat00078966 | g168747 | phytoene dehydrogenase |
| 1662 | rat00120521 | g168747 | phytoene dehydrogenase |
| 1663 | rat00213144 | g168747 | phytoene dehydrogenase |
| 1664 | rat00152143 | g168747 | phytoene dehydrogenase |
| 1665 | rat00203903 | g168747 | phytoene dehydrogenase |
| 1666 | rat00025460 | g168747 | phytoene dehydrogenase |
| 1667 | rat00082034 | g168747 | phytoene dehydrogenase |
| 1668 | rat00052515 | g168747 | phytoene dehydrogenase |
| 1669 | rat00117711 | g168747 | phytoene dehydrogenase |
| 1670 | rat00248310 | g168747 | phytoene dehydrogenase |
| 1671 | rat00123355 | g168747 | phytoene dehydrogenase |
| 1672 | rat00005860 | g168747 | phytoene dehydrogenase |
| 1673 | rat00217740 | g168747 | phytoene dehydrogenase |
| 1674 | rat00021189 | g168747 | phytoene dehydrogenase |
| 1675 | rat00111777 | g168747 | phytoene dehydrogenase |
| 1676 | rat00157434 | g168747 | phytoene dehydrogenase |
| 1677 | rat00154482 | g168747 | phytoene dehydrogenase |
| 1678 | rat00138760 | g168747 | phytoene dehydrogenase |
| 1679 | rat00188046 | g168747 | phytoene dehydrogenase |
| 1680 | rat00255844 | g168747 | phytoene dehydrogenase |
| 1681 | rat00236299 | g168747 | phytoene dehydrogenase |
| 1682 | rat00143999 | g168747 | phytoene dehydrogenase |
| 1683 | rat00238796 | g168747 | phytoene dehydrogenase |
| 1684 | rat00040500 | g168747 | phytoene dehydrogenase |
| 1685 | rat00001036 | g168747 | phytoene dehydrogenase |
| 1686 | rat00227113 | g168747 | phytoene dehydrogenase |
| 1687 | rat00157121 | g168747 | phytoene dehydrogenase |
| 1688 | rat00200366 | g168747 | phytoene dehydrogenase |
| 1689 | rat00236142 | g168747 | phytoene dehydrogenase |
| 1690 | rat00183042 | g168747 | phytoene dehydrogenase |
| 1691 | rat00016722 | g168747 | phytoene dehydrogenase |
| 1692 | rat00061390 | g168747 | phytoene dehydrogenase |
| 1693 | rat00193403 | g168747 | phytoene dehydrogenase |
| 1694 | rat00109704 | g168747 | phytoene dehydrogenase |
| 1695 | rat00176071 | g168747 | phytoene dehydrogenase |
| 1696 | rat00141862 | g168747 | phytoene dehydrogenase |
| 1697 | rat00155543 | g168747 | phytoene dehydrogenase |
| 1698 | rat00036447 | g168747 | phytoene dehydrogenase |
| 1699 | rat00248561 | g168747 | phytoene dehydrogenase |
| 1700 | rat00095634 | g168747 | phytoene dehydrogenase |
| 1701 | rat00233793 | g168747 | phytoene dehydrogenase |
| 1702 | rat00251696 | g168747 | phytoene dehydrogenase |
| 1703 | rat00098747 | g168747 | phytoene dehydrogenase |
| 1704 | rat00174779 | g168747 | phytoene dehydrogenase |
| 1705 | rat00032013 | g168747 | phytoene dehydrogenase |
| 1706 | rat00130024 | g168747 | phytoene dehydrogenase |
| 1707 | rat00199788 | g168747 | phytoene dehydrogenase |
| 1708 | rat00040710 | g168747 | phytoene dehydrogenase |
| 1709 | rat00237200 | g168747 | phytoene dehydrogenase |
| 1710 | rat00102606 | g168747 | phytoene dehydrogenase |
| 1711 | rat00116043 | g168747 | phytoene dehydrogenase |
| 1712 | rat00193013 | g168747 | phytoene dehydrogenase |
| 1713 | rat00193411 | g168747 | phytoene dehydrogenase |
| 1714 | rat00058115 | g168747 | phytoene dehydrogenase |
| 1715 | rat00158342 | g168747 | phytoene dehydrogenase |
| 1716 | rat00186206 | g168747 | phytoene dehydrogenase |
| 1717 | rat00062852 | g168747 | phytoene dehydrogenase |
| 1718 | rat00175712 | g168747 | phytoene dehydrogenase |
| 1719 | rat00005882 | g168747 | phytoene dehydrogenase |
| 1720 | rat00223962 | g168747 | phytoene dehydrogenase |
| 1721 | rat00224772 | g168747 | phytoene dehydrogenase |
| 1722 | rat00051487 | g168747 | phytoene dehydrogenase |
| 1723 | rat00154780 | g168747 | phytoene dehydrogenase |
| 1724 | rat00154826 | g168747 | phytoene dehydrogenase |
| 1725 | rat00059481 | g168747 | phytoene dehydrogenase |
| 1726 | rat00223849 | g168747 | phytoene dehydrogenase |
| 1727 | rat00157296 | g168747 | phytoene dehydrogenase |
| 1728 | rat00057549 | g168747 | phytoene dehydrogenase |
| 1729 | rat00167098 | g168747 | phytoene dehydrogenase |
| 1730 | rat00147858 | g168747 | phytoene dehydrogenase |
| 1731 | rat00063988 | g168747 | phytoene dehydrogenase |
| 1732 | rat00000043 | g168747 | phytoene dehydrogenase |
| 1733 | rat00130034 | g168747 | phytoene dehydrogenase |
| 1734 | rat00123222 | g168747 | phytoene dehydrogenase |
| 1735 | rat00002308 | g168747 | phytoene dehydrogenase |
| 1736 | rat00205115 | g168747 | phytoene dehydrogenase |
| 1737 | rat00130832 | g168747 | phytoene dehydrogenase |
| 1738 | rat00191402 | g168747 | phytoene dehydrogenase |
| 1739 | rat00044081 | g168747 | phytoene dehydrogenase |
| 1740 | rat00065250 | g168747 | phytoene dehydrogenase |
| 1741 | rat00016704 | g168747 | phytoene dehydrogenase |
| 1742 | rat00100918 | g168747 | phytoene dehydrogenase |
| 1743 | rat00205552 | g168747 | phytoene dehydrogenase |
| 1744 | rat00108751 | g168747 | phytoene dehydrogenase |
| 1745 | rat00250825 | g168747 | phytoene dehydrogenase |
| 1746 | rat00124790 | g168747 | phytoene dehydrogenase |
| 1747 | rat00140918 | g168747 | phytoene dehydrogenase |
| 1748 | rat00049451 | g168747 | phytoene dehydrogenase |
| 1749 | rat00139362 | g168747 | phytoene dehydrogenase |
| 1750 | rat00172338 | g168747 | phytoene dehydrogenase |
| 1751 | rat00207294 | g168747 | phytoene dehydrogenase |
| 1752 | hu00859025 | g476590 | Human cytochrome B561, HCYTO B561, mRNA, partial cds. |
| 1753 | hu00151711 | g476590 | Human cytochrome B561, HCYTO B561, mRNA, partial cds. |
| 1754 | hu00268925 | g476590 | Human cytochrome B561, HCYTO B561, mRNA, partial cds. |
| 1755 | hu00873888 | g476590 | Human cytochrome B561, HCYTO B561, mRNA, partial cds. |
| 1756 | rat00196343 | g30303 | precursor (AA −84 to 241) |
| 1757 | rat00162259 | g181240 | cytochrome c-1 |
| 1758 | rat00149803 | g181240 | cytochrome c-1 |
| 1759 | rat00047971 | g181240 | cytochrome c-1 |
| 1760 | rat00057844 | g181240 | cytochrome c-1 |
| 1761 | rat00096841 | g181240 | cytochrome c-1 |
| 1762 | rat00058693 | g181240 | cytochrome c-1 |
| 1763 | rat00161304 | g181240 | cytochrome c-1 |
| 1764 | rat00188358 | g181240 | cytochrome c-1 |
| 1765 | rat00204174 | g181240 | cytochrome c-1 |
| 1766 | rat00235404 | g181240 | cytochrome c-1 |
| 1767 | rat00144852 | g181240 | cytochrome c-1 |
| 1768 | rat00062953 | g181238 | cytochrome c1 |
| 1769 | rat00192832 | g181238 | cytochrome c1 |
| 1770 | rat00039681 | g181238 | cytochrome c1 |
| 1771 | rat00115488 | g181238 | cytochrome c1 |
| 1772 | rat00193491 | g181238 | cytochrome c1 |
| 1773 | rat00167807 | g181238 | cytochrome c1 |
| 1774 | rat00086790 | g181238 | cytochrome c1 |
| 1775 | rat00199525 | g181238 | cytochrome c1 |
| 1776 | rat00257665 | g181238 | cytochrome c1 |
| 1777 | rat00158777 | g181238 | cytochrome c1 |
| 1778 | rat00102523 | g181238 | cytochrome c1 |
| 1779 | rat00202063 | g181238 | cytochrome c1 |
| 1780 | rat00067043 | g181238 | cytochrome c1 |
| 1781 | rat00252273 | g181238 | cytochrome c1 |
| 1782 | rat00060259 | g181238 | cytochrome c1 |
| 1783 | rat00027860 | g181238 | cytochrome c1 |
| 1784 | rat00055734 | g181238 | cytochrome c1 |
| 1785 | rat00258360 | g181238 | cytochrome c1 |
| 1786 | rat00166056 | g181238 | cytochrome c1 |
| 1787 | rat00152547 | g181238 | cytochrome c1 |
| 1788 | hu01041273 | g30302 | Human mRNA for cytochrome c1. |
| 1789 | hu01084118 | g181239 | Human cytochrome c-1 gene, complete cds. |
| 1790 | hu01216492 | g181239 | Human cytochrome c-1 gene, complete cds. |
| 1791 | hu01229687 | g30302 | Human mRNA for cytochrome c1. |
| 1792 | hu01067795 | g30302 | Human mRNA for cytochrome c1. |
| 1793 | hu00967356 | g30302 | Human mRNA for cytochrome c1. |
| 1794 | hu00989750 | g30302 | Human mRNA for cytochrome c1. |
| 1795 | hu01310232 | g30302 | Human mRNA for cytochrome c1. |
| 1796 | hu01041413 | g30302 | Human mRNA for cytochrome c1. |
| 1797 | hu01280112 | g30302 | Human mRNA for cytochrome c1. |
| 1798 | hu01312017 | g30302 | Human mRNA for cytochrome c1. |
| 1799 | hu01303116 | g30302 | Human mRNA for cytochrome c1. |
| 1800 | hu01214233 | g30302 | Human mRNA for cytochrome c1. |
| 1801 | hu01217015 | g30302 | Human mRNA for cytochrome c1. |
| 1802 | hu01049111 | g30302 | Human mRNA for cytochrome c1. |
| 1803 | hu01107530 | g30302 | Human mRNA for cytochrome c1. |
| 1804 | hu01136440 | g30302 | Human mRNA for cytochrome c1. |
| 1805 | hu01220068 | g30302 | Human mRNA for cytochrome c1. |
| 1806 | hu01141615 | g30302 | Human mRNA for cytochrome c1. |
| 1807 | hu01062073 | g30302 | Human mRNA for cytochrome c1. |
| 1808 | hu01100792 | g30302 | Human mRNA for cytochrome c1. |
| 1809 | hu01001783 | g30302 | Human mRNA for cytochrome c1. |
| 1810 | hu01238043 | g30302 | Human mRNA for cytochrome c1. |
| 1811 | hu01090714 | g30302 | Human mRNA for cytochrome c1. |
| 1812 | hu01304754 | g30302 | Human mRNA for cytochrome c1. |
| 1813 | hu01214188 | g30302 | Human mRNA for cytochrome c1. |
| 1814 | hu01085182 | g30302 | Human mRNA for cytochrome c1. |
| 1815 | hu01104677 | g30302 | Human mRNA for cytochrome c1. |
| 1816 | hu01065329 | g30302 | Human mRNA for cytochrome c1. |
| 1817 | hu01315259 | g30302 | Human mRNA for cytochrome c1. |
| 1818 | hu01184635 | g30302 | Human mRNA for cytochrome c1. |
| 1819 | hu01261756 | g30302 | Human mRNA for cytochrome c1. |
| 1820 | hu01318645 | g30302 | Human mRNA for cytochrome c1. |
| 1821 | hu01284328 | g30302 | Human mRNA for cytochrome c1. |
| 1822 | hu01175852 | g30302 | Human mRNA for cytochrome c1. |
| 1823 | hu01265078 | g30302 | Human mRNA for cytochrome c1. |
| 1824 | hu01189523 | g30302 | Human mRNA for cytochrome c1. |
| 1825 | hu01291278 | g30302 | Human mRNA for cytochrome c1. |
| 1826 | hu01250699 | g30302 | Human mRNA for cytochrome c1. |
| 1827 | hu01281848 | g30302 | Human mRNA for cytochrome c1. |
| 1828 | hu00971781 | g30302 | Human mRNA for cytochrome c1. |
| 1829 | hu00271788 | g1651201 | cytochrome b561 |
| 1830 | rat00189898 | g1809118 | Rat thioredoxin mRNA, nuclear gene encoding mitochondrial protein, complete cds. |
| 1831 | rat00167530 | g1809118 | Rat thioredoxin mRNA, nuclear gene encoding mitochondrial protein, complete cds. |
| 1832 | rat00018842 | g1809118 | Rat thioredoxin mRNA, nuclear gene encoding mitochondrial protein, complete cds. |
| 1833 | rat00199561 | g1809118 | Rat thioredoxin mRNA, nuclear gene encoding mitochondrial protein, complete cds. |
| 1834 | rat00164141 | g1809118 | Rat thioredoxin mRNA, nuclear gene encoding mitochondrial protein, complete cds. |
| 1835 | rat00012080 | g1809118 | Rat thioredoxin mRNA, nuclear gene encoding mitochondrial protein, complete cds. |
| 1836 | rat00061336 | g1809118 | Rat thioredoxin mRNA, nuclear gene encoding mitochondrial protein, complete cds. |
| 1837 | rat00171128 | g1809118 | Rat thioredoxin mRNA, nuclear gene encoding mitochondrial protein, complete cds. |
| 1838 | rat00104497 | g2253160 | Rat mRNA for mitochondrial isoform of cytochrome b5. |
| 1839 | rat00241074 | g2253160 | Rat mRNA for mitochondrial isoform of cytochrome b5. |
| 1840 | rat00052948 | g2253160 | Rat mRNA for mitochondrial isoform of cytochrome b5. |
| 1841 | hu00408896 | g181241 | Human somatic cytochrome c (HCS) gene, complete cds. |
| 1842 | hu00686297 | g181241 | Human somatic cytochrome c (HCS) gene, complete cds. |
| 1843 | hu00888266 | g181241 | Human somatic cytochrome c (HCS) gene, complete cds. |
| 1844 | hu00612156 | g181241 | Human somatic cytochrome c (HCS) gene, complete cds. |
| 1845 | hu00404328 | g181241 | Human somatic cytochrome c (HCS) gene, complete cds. |
| 1846 | hu00052455 | g181241 | Human somatic cytochrome c (HCS) gene, complete cds. |
| 1847 | hu00124465 | g181241 | Human somatic cytochrome c (HCS) gene, complete cds. |
| 1848 | hu00494520 | g181241 | Human somatic cytochrome c (HCS) gene, complete cds. |
| 1849 | hu00808027 | g181241 | Human somatic cytochrome c (HCS) gene, complete cds. |
| 1850 | hu00025766 | g181241 | Human somatic cytochrome c (HCS) gene, complete cds. |
| 1851 | hu00453158 | g181241 | Human somatic cytochrome c (HCS) gene, complete cds. |
| 1852 | hu00316197 | g181241 | Human somatic cytochrome c (HCS) gene, complete cds. |
| 1853 | hu00087490 | g181241 | Human somatic cytochrome c (HCS) gene, complete cds. |
| 1854 | hu00475372 | g181241 | Human somatic cytochrome c (HCS) gene, complete cds. |
| 1855 | hu00616168 | g181241 | Human somatic cytochrome c (HCS) gene, complete cds. |
| 1856 | hu00080866 | g181241 | Human somatic cytochrome c (HCS) gene, complete cds. |
| 1857 | hu00751458 | g181241 | Human somatic cytochrome c (HCS) gene, complete cds. |
| 1858 | hu00960726 | g181241 | Human somatic cytochrome c (HCS) gene, complete cds. |
| 1859 | hu00610689 | g181241 | Human somatic cytochrome c (HCS) gene, complete cds. |
| 1860 | hu00557338 | g181241 | Human somatic cytochrome c (HCS) gene, complete cds. |
| 1861 | hu00357907 | g181241 | Human somatic cytochrome c (HCS) gene, complete cds. |
| 1862 | hu00694950 | g181241 | Human somatic cytochrome c (HCS) gene, complete cds. |
| 1863 | hu00535094 | g182250 | Human electron transfer flavoprotein alpha-subunit mRNA, complete cds. |
| 1864 | hu00343557 | g297901 | Human mRNA for electron transfer flavoprotein beta subunit. |
| 1865 | hu00367821 | g297901 | Human mRNA for electron transfer flavoprotein beta subunit. |
| 1866 | hu00475174 | g297901 | Human mRNA for electron transfer flavoprotein beta subunit. |
| 1867 | hu00165856 | g297901 | Human mRNA for electron transfer flavoprotein beta subunit. |
| 1868 | hu00177432 | g297901 | Human mRNA for electron transfer flavoprotein beta subunit. |
| 1869 | hu00309327 | g297901 | Human mRNA for electron transfer flavoprotein beta subunit. |
| 1870 | rat00077183 | g204069 | Rat electron transfer flavoprotein (ETF) alpha-subunit DNA, 3′ end. |
| 1871 | rat00174770 | g204069 | Rat electron transfer flavoprotein (ETF) alpha-subunit DNA, 3′ end. |
| 1872 | rat00181648 | g204069 | Rat electron transfer flavoprotein (ETF) alpha-subunit DNA, 3′ end. |
| 1873 | rat00231043 | g204069 | Rat electron transfer flavoprotein (ETF) alpha-subunit DNA, 3′ end. |
| 1874 | rat00241986 | g204069 | Rat electron transfer flavoprotein (ETF) alpha-subunit DNA, 3′ end. |
| 1875 | rat00204820 | g204069 | Rat electron transfer flavoprotein (ETF) alpha-subunit DNA, 3′ end. |
| 1876 | rat00211971 | g204069 | Rat electron transfer flavoprotein (ETF) alpha-subunit DNA, 3′ end. |
| 1877 | rat00156450 | g204069 | Rat electron transfer flavoprotein (ETF) alpha-subunit DNA, 3′ end. |
| 1878 | rat00058253 | g204069 | Rat electron transfer flavoprotein (ETF) alpha-subunit DNA, 3′ end. |
| 1879 | hu01333685 | g297901 | Human mRNA for electron transfer flavoprotein beta subunit. |
| 1880 | hu01102315 | g297901 | Human mRNA for electron transfer flavoprotein beta subunit. |
| 1881 | rat00212947 | g297902 | electron transfer flavoprotein beta subunit |
| 1882 | rat00209119 | g297902 | electron transfer flavoprotein beta subunit |
| 1883 | rat00093295 | g297902 | electron transfer flavoprotein beta subunit |
| 1884 | rat00083975 | g297902 | electron transfer flavoprotein beta subunit |
| 1885 | rat00139739 | g297902 | electron transfer flavoprotein beta subunit |
| 1886 | rat00194974 | g297902 | electron transfer flavoprotein beta subunit |
| 1887 | rat00002544 | g297902 | electron transfer flavoprotein beta subunit |
| 1888 | rat00016900 | g297902 | electron transfer flavoprotein beta subunit |
| 1889 | rat00204309 | g297902 | electron transfer flavoprotein beta subunit |
| 1890 | rat00181530 | g297902 | electron transfer flavoprotein beta subunit |
| 1891 | rat00161335 | g297902 | electron transfer flavoprotein beta subunit |
| 1892 | rat00190334 | g297902 | electron transfer flavoprotein beta subunit |
| 1893 | rat00010973 | g297902 | electron transfer flavoprotein beta subunit |
| 1894 | rat00254820 | g297902 | electron transfer flavoprotein beta subunit |
| 1895 | rat00013368 | g297902 | electron transfer flavoprotein beta subunit |
| 1896 | rat00061429 | g297902 | electron transfer flavoprotein beta subunit |
| 1897 | rat00240367 | g297902 | electron transfer flavoprotein beta subunit |
| 1898 | rat00006187 | g297902 | electron transfer flavoprotein beta subunit |
| 1899 | rat00099014 | g297902 | electron transfer flavoprotein beta subunit |
| 1900 | rat00219642 | g297902 | electron transfer flavoprotein beta subunit |
| 1901 | hu01127888 | g297902 | electron transfer flavoprotein beta subunit |
| 1902 | hu01039949 | g297902 | electron transfer flavoprotein beta subunit |
| 1903 | hu01217791 | g297902 | electron transfer flavoprotein beta subunit |
| 1904 | hu01202771 | g297902 | electron transfer flavoprotein beta subunit |
| 1905 | hu01303061 | g297902 | electron transfer flavoprotein beta subunit |
| 1906 | hu00983650 | g297902 | electron transfer flavoprotein beta subunit |
| 1907 | hu01326455 | g297902 | electron transfer flavoprotein beta subunit |
| 1908 | hu01279665 | g297901 | Human mRNA for electron transfer flavoprotein beta subunit. |
| 1909 | hu00989424 | g297901 | Human mRNA for electron transfer flavoprotein beta subunit. |
| 1910 | hu00992872 | g297901 | Human mRNA for electron transfer flavoprotein beta subunit. |
| 1911 | hu01163899 | g297901 | Human mRNA for electron transfer flavoprotein beta subunit. |
| 1912 | hu01187866 | g297901 | Human mRNA for electron transfer flavoprotein beta subunit. |
| 1913 | hu01309899 | g297901 | Human mRNA for electron transfer flavoprotein beta subunit. |
| 1914 | hu01100346 | g297901 | Human mRNA for electron transfer flavoprotein beta subunit. |
| 1915 | hu01229210 | g297901 | Human mRNA for electron transfer flavoprotein beta subunit. |
| 1916 | hu01333503 | g297901 | Human mRNA for electron transfer flavoprotein beta subunit. |
| 1917 | hu01143532 | g297901 | Human mRNA for electron transfer flavoprotein beta subunit. |
| 1918 | hu01090190 | g297901 | Human mRNA for electron transfer flavoprotein beta subunit. |
| 1919 | hu01142734 | g297901 | Human mRNA for electron transfer flavoprotein beta subunit. |
| 1920 | hu01341626 | g297901 | Human mRNA for electron transfer flavoprotein beta subunit. |
| 1921 | hu01047660 | g297901 | Human mRNA for electron transfer flavoprotein beta subunit. |
| 1922 | hu01040569 | g297901 | Human mRNA for electron transfer flavoprotein beta subunit. |
| 1923 | hu01118167 | g297901 | Human mRNA for electron transfer flavoprotein beta subunit. |
| 1924 | hu01341962 | g297901 | Human mRNA for electron transfer flavoprotein beta subunit. |
| 1925 | hu01076606 | g297901 | Human mRNA for electron transfer flavoprotein beta subunit. |
| 1926 | hu01246553 | g297901 | Human mRNA for electron transfer flavoprotein beta subunit. |
| 1927 | hu01308294 | g297901 | Human mRNA for electron transfer flavoprotein beta subunit. |
| 1928 | hu01253673 | g297901 | Human mRNA for electron transfer flavoprotein beta subunit. |
| 1929 | hu01018949 | g297901 | Human mRNA for electron transfer flavoprotein beta subunit. |
| 1930 | hu01295952 | g297901 | Human mRNA for electron transfer flavoprotein beta subunit. |
| 1931 | hu01186387 | g297901 | Human mRNA for electron transfer flavoprotein beta subunit. |
| 1932 | hu01292741 | g182250 | Human electron transfer flavoprotein alpha-subunit mRNA, complete cds. |
| 1933 | hu01133657 | g182250 | Human electron transfer flavoprotein alpha-subunit mRNA, complete cds. |
| 1934 | hu00995052 | g182250 | Human electron transfer flavoprotein alpha-subunit mRNA, complete cds. |
| 1935 | hu01265696 | g1809134 | Human thioredoxin mRNA, nuclear gene encoding mitochondrial protein, complete cds. |
| 1936 | hu00990768 | g1809134 | Human thioredoxin mRNA, nuclear gene encoding mitochondrial protein, complete cds. |
| 1937 | hu01317320 | g1809134 | Human thioredoxin mRNA, nuclear gene encoding mitochondrial protein, complete cds. |
| 1938 | hu01242531 | g1809134 | Human thioredoxin mRNA, nuclear gene encoding mitochondrial protein, complete cds. |
| 1939 | hu01037206 | g1809134 | Human thioredoxin mRNA, nuclear gene encoding mitochondrial protein, complete cds. |
| 1940 | hu01331119 | g1809134 | Human thioredoxin mRNA, nuclear gene encoding mitochondrial protein, complete cds. |
| 1941 | hu01052058 | g1809134 | Human thioredoxin mRNA, nuclear gene encoding mitochondrial protein, complete cds. |
| 1942 | hu01113646 | g1809134 | Human thioredoxin mRNA, nuclear gene encoding mitochondrial protein, complete cds. |
| 1943 | hu01271048 | g1809134 | Human thioredoxin mRNA, nuclear gene encoding mitochondrial protein, complete cds. |
| 1944 | hu01204349 | g1809134 | Human thioredoxin mRNA, nuclear gene encoding mitochondrial protein, complete cds. |
| 1945 | hu01252447 | g1809134 | Human thioredoxin mRNA, nuclear gene encoding mitochondrial protein, complete cds. |
| 1946 | hu01145870 | g1809134 | Human thioredoxin mRNA, nuclear gene encoding mitochondrial protein, complete cds. |
| 1947 | hu01048355 | g1809134 | Human thioredoxin mRNA, nuclear gene encoding mitochondrial protein, complete cds. |
| 1948 | hu01314199 | g1809134 | Human thioredoxin mRNA, nuclear gene encoding mitochondrial protein, complete cds. |
| 1949 | hu01329725 | g1809134 | Human thioredoxin mRNA, nuclear gene encoding mitochondrial protein, complete cds. |
| 1950 | hu01317971 | g1809134 | Human thioredoxin mRNA, nuclear gene encoding mitochondrial protein, complete cds. |
| 1951 | hu01254829 | g1809134 | Human thioredoxin mRNA, nuclear gene encoding mitochondrial protein, complete cds. |
| 1952 | hu01188249 | g1809134 | Human thioredoxin mRNA, nuclear gene encoding mitochondrial protein, complete cds. |
| 1953 | hu01258593 | g1809134 | Human thioredoxin mRNA, nuclear gene encoding mitochondrial protein, complete cds. |
| 1954 | hu01208697 | g1809134 | Human thioredoxin mRNA, nuclear gene encoding mitochondrial protein, complete cds. |
| 1955 | hu00979522 | g1809134 | Human thioredoxin mRNA, nuclear gene encoding mitochondrial protein, complete cds. |
| 1956 | hu01322638 | g1809134 | Human thioredoxin mRNA, nuclear gene encoding mitochondrial protein, complete cds. |
| 1957 | hu00579220 | g297901 | Human mRNA for electron transfer flavoprotein beta subunit. |
| 1958 | hu00278027 | g297901 | Human mRNA for electron transfer flavoprotein beta subunit. |
| 1959 | hu00150005 | g297901 | Human mRNA for electron transfer flavoprotein beta subunit. |
| 1960 | hu00435663 | g297901 | Human mRNA for electron transfer flavoprotein beta subunit. |
| 1961 | rat00104762 | g204069 | Rat electron transfer flavoprotein (ETF) alpha-subunit DNA, 3′ end. |
| 1962 | hu01179863 | g181242 | cytochrome c |
| 1963 | hu01016211 | g181242 | cytochrome c |
| 1964 | hu01003305 | g181242 | cytochrome c |
| 1965 | hu00977589 | g181242 | cytochrome c |
| 1966 | hu01130225 | g181242 | cytochrome c |
| 1967 | hu01205304 | g181242 | cytochrome c |
| 1968 | hu01017422 | g181242 | cytochrome c |
| 1969 | hu01200377 | g181242 | cytochrome c |
| 1970 | hu01067225 | g181242 | cytochrome c |
| 1971 | hu01084631 | g181242 | cytochrome c |
| 1972 | hu01047466 | g181242 | cytochrome c |
| 1973 | hu01343872 | g181242 | cytochrome c |
| 1974 | hu01077823 | g181242 | cytochrome c |
| 1975 | hu01326827 | g181242 | cytochrome c |
| 1976 | hu01348805 | g181242 | cytochrome c |
| 1977 | hu01090839 | g181242 | cytochrome c |
| 1978 | hu01090596 | g181242 | cytochrome c |
| 1979 | hu01325858 | g181242 | cytochrome c |
| 1980 | hu01279463 | g181242 | cytochrome c |
| 1981 | hu00974569 | g181242 | cytochrome c |
| 1982 | hu01092102 | g181242 | cytochrome c |
| 1983 | hu01335302 | g181242 | cytochrome c |
| 1984 | hu01172132 | g181242 | cytochrome c |
| 1985 | hu01027317 | g181242 | cytochrome c |
| 1986 | hu01312473 | g181242 | cytochrome c |
| 1987 | hu01253290 | g181242 | cytochrome c |
| 1988 | hu01003675 | g181242 | cytochrome c |
| 1989 | hu01124392 | g181242 | cytochrome c |
| 1990 | hu01311863 | g181242 | cytochrome c |
| 1991 | hu01192335 | g181242 | cytochrome c |
| 1992 | hu01327723 | g181242 | cytochrome c |
| 1993 | hu01287332 | g181242 | cytochrome c |
| 1994 | hu01045376 | g181242 | cytochrome c |
| 1995 | hu01017559 | g181242 | cytochrome c |
| 1996 | hu01055819 | g181242 | cytochrome c |
| 1997 | hu01070968 | g181242 | cytochrome c |
| 1998 | hu01326993 | g181242 | cytochrome c |
| 1999 | hu01196696 | g181242 | cytochrome c |
| 2000 | hu01168994 | g181242 | cytochrome c |
| 2001 | hu01071100 | g181242 | cytochrome c |
| 2002 | hu01071102 | g181242 | cytochrome c |
| 2003 | hu01014015 | g181242 | cytochrome c |
| 2004 | hu01141219 | g181242 | cytochrome c |
| 2005 | hu01317018 | g181242 | cytochrome c |
| 2006 | hu01117390 | g181242 | cytochrome c |
| 2007 | hu01062077 | g181242 | cytochrome c |
| 2008 | hu01216176 | g181242 | cytochrome c |
| 2009 | hu01155094 | g181242 | cytochrome c |
| 2010 | hu01022992 | g181242 | cytochrome c |
| 2011 | hu01208276 | g181242 | cytochrome c |
| 2012 | hu01150884 | g181242 | cytochrome c |
| 2013 | hu01282617 | g181242 | cytochrome c |
| 2014 | hu01331676 | g181242 | cytochrome c |
| 2015 | hu01148923 | g181242 | cytochrome c |
| 2016 | hu01179335 | g181242 | cytochrome c |
| 2017 | hu01132985 | g181242 | cytochrome c |
| 2018 | hu01199336 | g181242 | cytochrome c |
| 2019 | hu01185693 | g181242 | cytochrome c |
| 2020 | hu00969471 | g181242 | cytochrome c |
| 2021 | hu00975597 | g181242 | cytochrome c |
| 2022 | hu01114017 | g181242 | cytochrome c |
| 2023 | hu01013290 | g181242 | cytochrome c |
| 2024 | hu01224206 | g181242 | cytochrome c |
| 2025 | hu01211615 | g181242 | cytochrome c |
| 2026 | hu01141146 | g181242 | cytochrome c |
| 2027 | hu01284800 | g181242 | cytochrome c |
| 2028 | hu01207517 | g181242 | cytochrome c |
| 2029 | hu01247630 | g181242 | cytochrome c |
| 2030 | hu01060945 | g181242 | cytochrome c |
| 2031 | hu01332655 | g181242 | cytochrome c |
| 2032 | hu01015795 | g181242 | cytochrome c |
| 2033 | hu01309880 | g181242 | cytochrome c |
| 2034 | hu01346890 | g181242 | cytochrome c |
| 2035 | hu01182595 | g181242 | cytochrome c |
| 2036 | rat00225961 | g801871 | Rat mRNA for adrenodoxin, complete cds. |
| 2037 | rat00027224 | g801871 | Rat mRNA for adrenodoxin, complete cds. |
| 2038 | rat00216641 | g801871 | Rat mRNA for adrenodoxin, complete cds. |
| 2039 | hu01057340 | g341002 | Human adrenodoxin gene, exon 4. |
| 2040 | hu00914886 | g182250 | Human electron transfer flavoprotein alpha-subunit mRNA, complete cds. |
| 2041 | hu00582409 | g182250 | Human electron transfer flavoprotein alpha-subunit mRNA, complete cds. |
| 2042 | hu00193205 | g182250 | Human electron transfer flavoprotein alpha-subunit mRNA, complete cds. |
| 2043 | hu00691687 | g182250 | Human electron transfer flavoprotein alpha-subunit mRNA, complete cds. |
| 2044 | hu00601763 | g182250 | Human electron transfer flavoprotein alpha-subunit mRNA, complete cds. |
| 2045 | hu00459499 | g182250 | Human electron transfer flavoprotein alpha-subunit mRNA, complete cds. |
| 2046 | hu00772054 | g182250 | Human electron transfer flavoprotein alpha-subunit mRNA, complete cds. |
| 2047 | hu00095625 | g182250 | Human electron transfer flavoprotein alpha-subunit mRNA, complete cds. |
| 2048 | hu00128036 | g182250 | Human electron transfer flavoprotein alpha-subunit mRNA, complete cds. |
| 2049 | hu00870833 | g182250 | Human electron transfer flavoprotein alpha-subunit mRNA, complete cds. |
| 2050 | hu00708205 | g182250 | Human electron transfer flavoprotein alpha-subunit mRNA, complete cds. |
| 2051 | rat00154045 | g550511 | Rat (Sprague-Dawley) cytochrome c nuclear-encoded mitochondrial gene and flanks. |
| 2052 | hu00875504 | g1809134 | Human thioredoxin mRNA, nuclear gene encoding mitochondrial protein, complete cds. |
| 2053 | hu00061898 | g1809134 | Human thioredoxin mRNA, nuclear gene encoding mitochondrial protein, complete cds. |
| 2054 | hu00853194 | g1809134 | Human thioredoxin mRNA, nuclear gene encoding mitochondrial protein, complete cds. |
| 2055 | hu00890790 | g1809134 | Human thioredoxin mRNA, nuclear gene encoding mitochondrial protein, complete cds. |
| 2056 | hu00760437 | g1809134 | Human thioredoxin mRNA, nuclear gene encoding mitochondrial protein, complete cds. |
| 2057 | hu00890998 | g1809134 | Human thioredoxin mRNA, nuclear gene encoding mitochondrial protein, complete cds. |
| 2058 | hu00152759 | g1809134 | Human thioredoxin mRNA, nuclear gene encoding mitochondrial protein, complete cds. |
| 2059 | hu00748297 | g1809134 | Human thioredoxin mRNA, nuclear gene encoding mitochondrial protein, complete cds. |
| 2060 | hu00958835 | g1809134 | Human thioredoxin mRNA, nuclear gene encoding mitochondrial protein, complete cds. |
| 2061 | hu00797565 | g1809134 | Human thioredoxin mRNA, nuclear gene encoding mitochondrial protein, complete cds. |
| 2062 | hu00023242 | g1809134 | Human thioredoxin mRNA, nuclear gene encoding mitochondrial protein, complete cds. |
| 2063 | hu00219001 | g1809134 | Human thioredoxin mRNA, nuclear gene encoding mitochondrial protein, complete cds. |
| 2064 | hu00270032 | g1809134 | Human thioredoxin mRNA, nuclear gene encoding mitochondrial protein, complete cds. |
| 2065 | hu00119012 | g1809134 | Human thioredoxin mRNA, nuclear gene encoding mitochondrial protein, complete cds. |
| 2066 | hu00160405 | g1809134 | Human thioredoxin mRNA, nuclear gene encoding mitochondrial protein, complete cds. |
| 2067 | hu00190208 | g1809134 | Human thioredoxin mRNA, nuclear gene encoding mitochondrial protein, complete cds. |
| 2068 | hu00626332 | g1809134 | Human thioredoxin mRNA, nuclear gene encoding mitochondrial protein, complete cds. |
| 2069 | hu00100687 | g1809134 | Human thioredoxin mRNA, nuclear gene encoding mitochondrial protein, complete cds. |
| 2070 | hu00320665 | g1809134 | Human thioredoxin mRNA, nuclear gene encoding mitochondrial protein, complete cds. |
| 2071 | hu00239361 | g1809134 | Human thioredoxin mRNA, nuclear gene encoding mitochondrial protein, complete cds. |
| 2072 | hu00920049 | g1809134 | Human thioredoxin mRNA, nuclear gene encoding mitochondrial protein, complete cds. |
| 2073 | hu00524608 | g1809134 | Human thioredoxin mRNA, nuclear gene encoding mitochondrial protein, complete cds. |
| 2074 | hu00364660 | g1809134 | Human thioredoxin mRNA, nuclear gene encoding mitochondrial protein, complete cds. |
| 2075 | hu00845262 | g1809134 | Human thioredoxin mRNA, nuclear gene encoding mitochondrial protein, complete cds. |
| 2076 | hu00633709 | g1809134 | Human thioredoxin mRNA, nuclear gene encoding mitochondrial protein, complete cds. |
| 2077 | hu00495146 | g1809134 | Human thioredoxin mRNA, nuclear gene encoding mitochondrial protein, complete cds. |
| 2078 | hu00913556 | g1809134 | Human thioredoxin mRNA, nuclear gene encoding mitochondrial protein, complete cds. |
| 2079 | hu00428818 | g1809134 | Human thioredoxin mRNA, nuclear gene encoding mitochondrial protein, complete cds. |
| 2080 | hu00565575 | g1809134 | Human thioredoxin mRNA, nuclear gene encoding mitochondrial protein, complete cds. |
| 2081 | hu00827367 | g1809134 | Human thioredoxin mRNA, nuclear gene encoding mitochondrial protein, complete cds. |
| 2082 | hu00786562 | g1809134 | Human thioredoxin mRNA, nuclear gene encoding mitochondrial protein, complete cds. |
| 2083 | hu00228878 | g1809134 | Human thioredoxin mRNA, nuclear gene encoding mitochondrial protein, complete cds. |
| 2084 | hu00356687 | g1809134 | Human thioredoxin mRNA, nuclear gene encoding mitochondrial protein, complete cds. |
| 2085 | hu00878932 | g1809134 | Human thioredoxin mRNA, nuclear gene encoding mitochondrial protein, complete cds. |
| 2086 | hu00501343 | g1809134 | Human thioredoxin mRNA, nuclear gene encoding mitochondrial protein, complete cds. |
| 2087 | hu00531225 | g1809134 | Human thioredoxin mRNA, nuclear gene encoding mitochondrial protein, complete cds. |
| 2088 | hu00100738 | g1809134 | Human thioredoxin mRNA, nuclear gene encoding mitochondrial protein, complete cds. |
| 2089 | hu00114571 | g1809134 | Human thioredoxin mRNA, nuclear gene encoding mitochondrial protein, complete cds. |
| 2090 | hu00161624 | g1809134 | Human thioredoxin mRNA, nuclear gene encoding mitochondrial protein, complete cds. |
| 2091 | hu00161626 | g1809134 | Human thioredoxin mRNA, nuclear gene encoding mitochondrial protein, complete cds. |
| 2092 | hu00534136 | g1809134 | Human thioredoxin mRNA, nuclear gene encoding mitochondrial protein, complete cds. |
| 2093 | hu00107681 | g1809134 | Human thioredoxin mRNA, nuclear gene encoding mitochondrial protein, complete cds. |
| 2094 | hu00257711 | g1809134 | Human thioredoxin mRNA, nuclear gene encoding mitochondrial protein, complete cds. |
| 2095 | hu00237192 | g1809134 | Human thioredoxin mRNA, nuclear gene encoding mitochondrial protein, complete cds. |
| 2096 | hu00770272 | g1809134 | Human thioredoxin mRNA, nuclear gene encoding mitochondrial protein, complete cds. |
| 2097 | hu00409950 | g1809134 | Human thioredoxin mRNA, nuclear gene encoding mitochondrial protein, complete cds. |
| 2098 | hu00467172 | g1809134 | Human thioredoxin mRNA, nuclear gene encoding mitochondrial protein, complete cds. |
| 2099 | hu00609932 | g1809134 | Human thioredoxin mRNA, nuclear gene encoding mitochondrial protein, complete cds. |
| 2100 | hu00318990 | g1809134 | Human thioredoxin mRNA, nuclear gene encoding mitochondrial protein, complete cds. |
| 2101 | hu00156990 | g1809134 | Human thioredoxin mRNA, nuclear gene encoding mitochondrial protein, complete cds. |
| 2102 | hu00791682 | g1809134 | Human thioredoxin mRNA, nuclear gene encoding mitochondrial protein, complete cds. |
| 2103 | hu00383668 | g1809134 | Human thioredoxin mRNA, nuclear gene encoding mitochondrial protein, complete cds. |
| 2104 | hu00956473 | g1809134 | Human thioredoxin mRNA, nuclear gene encoding mitochondrial protein, complete cds. |
| 2105 | hu00858068 | g1809134 | Human thioredoxin mRNA, nuclear gene encoding mitochondrial protein, complete cds. |
| 2106 | hu00774953 | g1809134 | Human thioredoxin mRNA, nuclear gene encoding mitochondrial protein, complete cds. |
| 2107 | hu00759728 | g1809134 | Human thioredoxin mRNA, nuclear gene encoding mitochondrial protein, complete cds. |
| 2108 | hu00889195 | g1809134 | Human thioredoxin mRNA, nuclear gene encoding mitochondrial protein, complete cds. |
| 2109 | hu00665977 | g1809134 | Human thioredoxin mRNA, nuclear gene encoding mitochondrial protein, complete cds. |
| 2110 | hu00520221 | g1809134 | Human thioredoxin mRNA, nuclear gene encoding mitochondrial protein, complete cds. |
| 2111 | hu00731210 | g1809134 | Human thioredoxin mRNA, nuclear gene encoding mitochondrial protein, complete cds. |
| 2112 | hu00005443 | g1809134 | Human thioredoxin mRNA, nuclear gene encoding mitochondrial protein, complete cds. |
| 2113 | hu00641224 | g1809134 | Human thioredoxin mRNA, nuclear gene encoding mitochondrial protein, complete cds. |
| 2114 | hu00117253 | g1809134 | Human thioredoxin mRNA, nuclear gene encoding mitochondrial protein, complete cds. |
| 2115 | hu00920305 | g1809134 | Human thioredoxin mRNA, nuclear gene encoding mitochondrial protein, complete cds. |
| 2116 | hu00363233 | g1809134 | Human thioredoxin mRNA, nuclear gene encoding mitochondrial protein, complete cds. |
| 2117 | hu00222753 | g1809134 | Human thioredoxin mRNA, nuclear gene encoding mitochondrial protein, complete cds. |
| 2118 | hu00394401 | g1809134 | Human thioredoxin mRNA, nuclear gene encoding mitochondrial protein, complete cds. |
| 2119 | hu00282154 | g1809134 | Human thioredoxin mRNA, nuclear gene encoding mitochondrial protein, complete cds. |
| 2120 | hu00183253 | g1809134 | Human thioredoxin mRNA, nuclear gene encoding mitochondrial protein, complete cds. |
| 2121 | hu00278213 | g1809134 | Human thioredoxin mRNA, nuclear gene encoding mitochondrial protein, complete cds. |
| 2122 | hu00091924 | g1809134 | Human thioredoxin mRNA, nuclear gene encoding mitochondrial protein, complete cds. |
| 2123 | hu00474271 | g1809134 | Human thioredoxin mRNA, nuclear gene encoding mitochondrial protein, complete cds. |
| 2124 | hu00260575 | g1809134 | Human thioredoxin mRNA, nuclear gene encoding mitochondrial protein, complete cds. |
| 2125 | hu00260692 | g1809134 | Human thioredoxin mRNA, nuclear gene encoding mitochondrial protein, complete cds. |
| 2126 | hu00882413 | g1809134 | Human thioredoxin mRNA, nuclear gene encoding mitochondrial protein, complete cds. |
| 2127 | hu00891127 | g1809134 | Human thioredoxin mRNA, nuclear gene encoding mitochondrial protein, complete cds. |
| 2128 | hu00198146 | g1809134 | Human thioredoxin mRNA, nuclear gene encoding mitochondrial protein, complete cds. |
| 2129 | hu00791651 | g1809134 | Human thioredoxin mRNA, nuclear gene encoding mitochondrial protein, complete cds. |
| 2130 | hu00924863 | g1809134 | Human thioredoxin mRNA, nuclear gene encoding mitochondrial protein, complete cds. |
| 2131 | hu00491649 | g1809134 | Human thioredoxin mRNA, nuclear gene encoding mitochondrial protein, complete cds. |
| 2132 | hu00739378 | g1809134 | Human thioredoxin mRNA, nuclear gene encoding mitochondrial protein, complete cds. |
| 2133 | hu00478991 | g1809134 | Human thioredoxin mRNA, nuclear gene encoding mitochondrial protein, complete cds. |
| 2134 | hu00820521 | g1809134 | Human thioredoxin mRNA, nuclear gene encoding mitochondrial protein, complete cds. |
| 2135 | hu00004088 | g1809134 | Human thioredoxin mRNA, nuclear gene encoding mitochondrial protein, complete cds. |
| 2136 | hu00019055 | g1809134 | Human thioredoxin mRNA, nuclear gene encoding mitochondrial protein, complete cds. |
| 2137 | hu00138630 | g1809134 | Human thioredoxin mRNA, nuclear gene encoding mitochondrial protein, complete cds. |
| 2138 | hu00411573 | g1809134 | Human thioredoxin mRNA, nuclear gene encoding mitochondrial protein, complete cds. |
| 2139 | hu00543181 | g1809134 | Human thioredoxin mRNA, nuclear gene encoding mitochondrial protein, complete cds. |
| 2140 | hu00407552 | g1809134 | Human thioredoxin mRNA, nuclear gene encoding mitochondrial protein, complete cds. |
| 2141 | hu00446342 | g1809134 | Human thioredoxin mRNA, nuclear gene encoding mitochondrial protein, complete cds. |
| 2142 | hu00374090 | g1809134 | Human thioredoxin mRNA, nuclear gene encoding mitochondrial protein, complete cds. |
| 2143 | hu00065058 | g1809134 | Human thioredoxin mRNA, nuclear gene encoding mitochondrial protein, complete cds. |
| 2144 | hu00484406 | g1809134 | Human thioredoxin mRNA, nuclear gene encoding mitochondrial protein, complete cds. |
| 2145 | hu00346157 | g1809134 | Human thioredoxin mRNA, nuclear gene encoding mitochondrial protein, complete cds. |
| 2146 | hu00428607 | g1809134 | Human thioredoxin mRNA, nuclear gene encoding mitochondrial protein, complete cds. |
| 2147 | hu00495415 | g1809134 | Human thioredoxin mRNA, nuclear gene encoding mitochondrial protein, complete cds. |
| 2148 | hu00722593 | g1809134 | Human thioredoxin mRNA, nuclear gene encoding mitochondrial protein, complete cds. |
| 2149 | hu00155683 | g1809134 | Human thioredoxin mRNA, nuclear gene encoding mitochondrial protein, complete cds. |
| 2150 | hu00839215 | g1809134 | Human thioredoxin mRNA, nuclear gene encoding mitochondrial protein, complete cds. |
| 2151 | hu00868081 | g1809134 | Human thioredoxin mRNA, nuclear gene encoding mitochondrial protein, complete cds. |
| 2152 | hu01322291 | g182250 | Human electron transfer flavoprotein alpha-subunit mRNA, complete cds. |
| 2153 | hu01053686 | g182250 | Human electron transfer flavoprotein alpha-subunit mRNA, complete cds. |
| 2154 | hu00984752 | g182251 | electron transport flavoprotein |
| 2155 | hu01121158 | g182251 | electron transport flavoprotein |
| 2156 | hu01069140 | g182251 | electron transport flavoprotein |
| 2157 | hu01289279 | g182251 | electron transport flavoprotein |
| 2158 | hu01220863 | g182251 | electron transport flavoprotein |
| 2159 | hu01044339 | g182251 | electron transport flavoprotein |
| 2160 | hu01286152 | g182251 | electron transport flavoprotein |
| 2161 | hu01336363 | g182251 | electron transport flavoprotein |
| 2162 | hu01118609 | g182251 | electron transport flavoprotein |
| 2163 | hu00992223 | g182251 | electron transport flavoprotein |
| 2164 | hu01142739 | g182251 | electron transport flavoprotein |
| 2165 | hu01345188 | g182251 | electron transport flavoprotein |
| 2166 | hu01199245 | g182251 | electron transport flavoprotein |
| 2167 | hu01205260 | g182251 | electron transport flavoprotein |
| 2168 | hu01232431 | g182251 | electron transport flavoprotein |
| 2169 | hu01136250 | g182251 | electron transport flavoprotein |
| 2170 | hu01156768 | g182251 | electron transport flavoprotein |
| 2171 | hu01020636 | g182251 | electron transport flavoprotein |

1. A purified mammalian polynucleotide comprising:
a) SEQ ID NOs:1-2171;
b) a fragment of at least 18 consecutive nucleotides selected from SEQ ID NOs:1-2171; and
c) a complement of a) or b).

2. A composition comprising at least one polynucleotide of claim 1.

3. An substrate comprising at least one polynucleotide of claim 1.

4. A probe comprising a polynucleotide of claim 1.

5. An expression vector comprising a polynucleotide of claim 1.

6. A host cell comprising the expression vector of claim 5.

7. A method for producing a polypeptide or a portion thereof, the method comprising:
a) culturing the host cell of claim 6 under conditions for the expression of the polypeptide; and
b) recovering the polypeptide from the cell culture.

8. A method for detecting a mammalian polynucleotide in a sample, the method comprising:
a) hybridizing the composition of claim 2 to at least one nucleic acid molecule in the sample, thereby forming a hybridization complex; and
b) detecting the hybridization complex, wherein the presence of the hybridization complex indicates the presence of the mammalian polynucleotide in the sample.

9. The method of claim 8 further comprising amplifying the nucleic acid molecules of the sample prior to hybridization.

10. A method of using a polynucleotide to screen a plurality of molecules or compounds to identify a ligand that specifically binds the polynucleotide, the method comprising:
a) combining a polynucleotide of claim 1 with a plurality of molecules or compounds under conditions to allow specific binding; and
b) detecting specific binding between the polynucleotide and a molecule or compound, thereby identifying a ligand that specifically binds the polynucleotide.

11. The method of claim 10 wherein the ligand is identified from DNA molecules, RNA molecules, PNAs, peptides, and proteins.

12. A transcription factor identified using the method of claim 8.

13. A method of using a polynucleotide to purify a ligand from a sample, the method comprising:
a) combining a polynucleotide of claim 1 with a sample under conditions to allow specific binding;
b) recovering the bound polynucleotide; and
c) separating the polynucleotide from the molecule or compound, thereby obtaining purified ligand.

14. A purified polypeptide or a portion thereof encoded by a polynucleotide of claim 1.

15. A composition comprising the polypeptide of claim 14.

16. A method for using a polypeptide to screen a plurality of molecules or compounds to identify a ligand that specifically binds the polypeptide, the method comprising:
a) combining the polypeptide of claim 14 with a plurality of molecules or compounds under conditions to allow specific binding; and
b) detecting specific binding between the polypeptide and a molecule or compound, thereby identifying a ligand that specifically binds the polypeptide.

17. The method of claim 16 wherein the ligand that specifically binds the polypeptide is selected from agonists, antagonists, antibodies, carbohydrates, DNA molecules, drug compounds, fatty acids, immunoglobulins, inhibitors, lipids, mimetics, morpholinos, peptide nucleic acids, peptides, pharmaceutical agents, proteins, RNA molecules, or ribozymes.

18. A method for using a polypeptide to produce an antibody, the method comprising:
a) immunizing an animal with the polypeptide of claim 14 or an antigenically-effective fragment thereof under conditions to elicit an antibody response;
b) isolating animal antibodies; and
c) screening the isolated antibodies with the polypeptide thereby identifying a polyclonal antibody binds specifically to the polypeptide.

19. An antibody used to diagnose a disorder associated with expression of the polynucleotide of claim 1.

20. A method of using a polypeptide to purify a ligand from a sample, the method comprising:
a) combining the polypeptide of claim 14 with the sample under conditions to allow specific binding;
b) recovering bound polypeptide; and
c) separating the polypeptide from the ligand, thereby obtaining purified ligand.
